(19) United States
(12) Patent Application Publication
(10) Pub. No.: US 2010/0048891 A1
(43) Pub. Date: Feb. 25, 2010

(54) METASUBSTITUTED THIAZOLIDINONES, THEIR MANUFACTURE AND USE AS A DRUG

(76) Inventors: Klause SCHULZE, Bergfelde (DE); Knut Eis, Berlin (DE); Lars Wortmann, Berlin (DE); Dirk Kosemund, Erfurt (DE); Olaf Prien, Berlin (DE); Gerhard Siemeister, Berlin (DE); Holger Hess-Stumpp, Berlin (DE); Uwe Eberspaecher, Berlin (DE); Dominic E.A. Brittain, Berlin (DE); Imadul Islam, Hercules, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/432,213

(22) Filed: Apr. 29, 2009

Related U.S. Application Data
(63) Continuation of application No. 11/302,537, filed on Dec. 14, 2005, now abandoned.
(60) Provisional application No. 60/637,777, filed on Dec. 22, 2004.

(30) Foreign Application Priority Data
Dec. 15, 2004 (DE) 102004061503.9

(51) Int. Cl. C07D 417/02; C07D 277/04; C07D 413/02; C07D 239/02

(52) U.S. Cl. 540/603; 548/187; 546/209; 544/369; 544/133; 546/269.7; 544/60; 544/323

(57) ABSTRACT

Thiazolidinones of general formula (I)

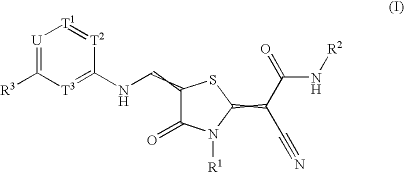

production thereof and use as inhibitors of polo like kinase (PLK) for the treatment of various diseases.

This application claims the benefit of the filing date of U.S. Provisional Application Ser. No. 60/637,777 filed Dec. 22, 2004.

The invention concerns thiazolidinones, their manufacture and use as polo-like kinase (PLK) inhibitors for treating various diseases.

Tumor cells set themselves apart through their uninhibited cell-cycle process. On the one hand, it is based on the loss of control proteins like RB, p16, p21, p53 etc. as well as the activation of so-called cell-cycle process accelerators, the cyclin-dependant kinases (CDK's). CDK's are a recognized anti-tumor target-protein in pharmacy. In addition to the CDK's, new cell-cycle regulating serine/threonine-kinases, so-called ‘polo-like kinases’ were described that are involved not only in regulating cell-cycles but also in coordinating with other processes during mitosis and cytokinesis (formation of the spindle apparatus, chromosome separation). Consequently, this protein class represents an interesting point of contact for the therapeutic intervention of proliferative diseases like cancer (Descombes and Nigg. Embo j, 17; 1328ff, 1998; Glover et al. Genes Dev 12, 3777ff, 1998).

A high expression rate of PLK-1 was found in ‘non-small cell lung’-cancer (Wolf et al. Oncogene, 14, 543ff, 1997), in melanomas (Strebhardt et al. JAMA, 283, 479ff, 2000), in ‘squamous cell carcinomas’ (Knecht et al. Cancer Res, 59, 2794ff, 1999) and in ‘esophageal carcinomas’ (Tokumitsu et al. Int J Oncol 15, 687ff, 1999).

A correlation of high expression rate was shown in tumor patients with a poor prognosis for sundry tumors (Strebhardt et al. JAMA, 283, 479ff, 2000, Knecht et al. Cancer Res, 59, 2794ff, 1999 and Tokumitsu et al. Int J Oncol 15, 687ff, 1999).

Constitutive expression of PLK-1 in NIH-3t3-cells resulted in malignant transformation (increased proliferation, soft-agar growth, colony formation, and tumor development in naked mice (Smith et al. Biochem Biophys Res Comm, 234, 397ff., 1997).

Microinjections of PLK-1-antibodies into HeLa-cells resulted in defective mitosis (Lane et al.; Journal Cell Biol, 135, 1701 ff, 1996).

Using a ‘20-mer’ antisense oligo the expression of PLK-1 in a549-cells could be inhibited and their ability to survive stopped. A clear anti-tumor-effect could also be demonstrated in naked mice (Mundt et al., Biochem Biophys Res Comm, 269, 377ff., 2000).

Microinjecting anti-PLK-antibodies into nonimmortalized human hs68-cells exhibited, in contrast to HeLa cells, significantly higher fraction of cells, which remained in a growth arrest on G2 and exhibited far fewer indications of defective mitosis (Lane et al.; Journal Cell Biol, 135, 1701ff, 1996).

In contrast to tumor cells, antisense-oligo-molecules did not inhibit the growth and viability of primary human mesangial cells (Mundt et al., Biochem Biophys Res Comm, 269, 377ff., 2000).

Until now, besides PLK-1, three other polo-kinases that are mitogenic response-induced and that perform their function in the G1 phase of the cell cycle, have been documented in mammals. They are the so-called PRK/PLK-3 (the human homologue of the mouse FNK=fibroblast growth factor induced kinase; Wiest et al, Genes, Chromosomes & Cancer, 32: 384ff, 2001), SNK/PLK-2 (serum induced kinase, Liby et al., DNA Sequence, 11, 527-33, 2001) and SAK/PLK4 (Fode et al., Proc. Natl. Acad. Sci. U.S.A., 91, 6388ff; 1994).

The inhibition of PLK-1 and the other kinases of the polo-family, like PLK-2, PLK-3 and PLK-4 therefore represent a promising approach for treating a variety of diseases.

The sequence identity within the PLK-domains of the polo-family lies between 40 and 60%, such that sometimes the inhibitors of one kinase will interact with one or several other kinases of that family. But depending on the structure of the inhibitor, the effect can also occur selectively or preferably on only kinase of the polo family.

International application WO 03/093249 discloses thiazolidinone compounds that inhibit kinases of the polo family.

The task of the present compound is to furnish improved compounds, improved particularly in the inhibition of polo-like kinases as compared to prior art and/or to provide compounds that inhibit kinases, in particular polo-like kinases or that have better physicochemical properties as compared to compounds disclosed in prior art.

### FIRST EMBODIMENT OF THE PRESENT INVENTION

In a first embodiment of the present invention it was found in claim 1 that compounds of the general formula I,

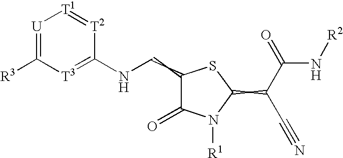

in which
T¹, T² and T³ independently represent —CH═ or —N═ and T² can also represent (—CF)═,
U represents —CR⁴═ or —N═,
R¹ represents C₁-C₃-alkyl or cyclopropyl optionally mono- or polysubstituted identically or differently with halogen,
R² represents C₁-C₃-alkyl, C₃-C₄-alkenyl, C₃-C₄-alkinyl or cyclopropyl optionally mono- or polysubstituted, identically or differently, with cyanogen, cyclopropyl, ethinyl or halogen, or hydroxyl, ethyl at least monosubstituted with methyl,
R³ represents K, L or M or R¹⁵,
K represents C₁-C₃-alkyl or C₂-C₄-alkenyl optionally mono- or polysubstituted, identically or differently, with X,
X represents halogen, hydroxyl or the group —OR⁶, —NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
L represents the group —O—R⁷, —O—(CH₂)ₙ—(CO)—NH—R⁸—O—(CH₂)ₙ—(CO)—R¹⁵ or —O—(CH₂)ₙ—(CO)—O—R⁸,
M represents the group —NH—R⁹, —NH—(CO)—OH, —NH—(CO)—O—R⁹ or —NR¹²—(CO)—R¹⁶,
R⁴ represents hydrogen, cyanogen or halogen or represents methyl optionally mono- or polysubstituted, identically or differently, with halogen,
R⁵ represents C₁-C₄-alkyl, phenyl or —NR¹²R¹³,
R⁶ represents —SO₂—R¹⁴,
R⁷ represents C₁-C₃-alkyl optionally mono- or polysubstituted, identically or differently, with —NR¹²R¹³ or C₂-C₁₀-heterocycloalkyl, wherein the heterocycloalkyl contains at least one atom, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, aryl or C₁-C₃-alkyl optionally mono- or polysubstituted, identically or differently, with halogen,
R⁸ represents C₁-C₃-alkyl, C₃-C₄-alkenyl or C₃-C₄-alkinyl optionally mono- or polysubstituted, identically or differently, with cyanogen, cyclopropyl or halogen,
R⁹ represents C₁-C₅-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can optionally be interrupted in the ring by one or several —(CO)— or —SO— groups and optionally one or several double bonds can be contained in the ring and the ring itself can be optionally mono- or polysubstituted, identically or differently, with halogen, cyanogen, hydroxyl, aryl or with the group —(CO)—R⁵—(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be mono- or polysubstituted, identically or differently, with halogen or C₁-C₃-alkoxyl,
R¹⁰ and R¹¹ independently represent C₁-C₅-alkyl, C₂-C₁₀-heterocycloalkyl, aryl, —(CH₂)ₙ-aryl or heteroaryl optionally mono- or polysubstituted, identically or differently, with halogen, C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
R¹² and R¹³ independently represent hydrogen or C₁-C₄-alkyl,
R¹⁴ represents C₁-C₃-alkyl or aryl
R¹⁵ represents C₂-C₁₀-heterocycloalkyl optionally mono- or polysubstituted, identically or differently, with C₁-C₃-alkyl or —(CH₂)ₙ-aryl, wherein the heterocycloalkyl contains at least one atom in the ring, identically or differently, from the group nitrogen, oxygen or sulfur, and can be optionally interrupted in the ring by one or several —(CO)— or —SO₂— groups and optionally one or several double bonds can be contained in the ring,
R¹⁶ represents hydrogen or C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or C₁-C₄-alkyl mono- or polysubstituted, identically or differently, with C₁-C₄-alkoxyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or represents methyl optionally mono- or polysubstituted, identically or differently, with C₂-C₁₀-heterocycloalkyl or heteroaryl, wherein the heterocycloalkyl contains at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl or C₁-C₃-alkoxyl,
   or represents C₁-C₄-alkyl mono- or polysubstituted, identically or differently, with C₂-C₁₀-heterocycloalkyl, or represents C₂-C₄-alkyl mono- or polysubstituted, identically or differently, with C₁-C₄-alkoxy-C₁-C₄-alkoxyl, wherein the heterocycloalkyl contains at least one atom in the ring, identically or differently, from the following group, oxygen or sulfur and can be optionally interrupted in the ring by one or several —(CO)—, —(C═S)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl or C₁-C₃-alkoxyl, and
n is 1-4,
and their solvates, hydrates, diastereomers, enantiomers and salts, are improved compounds where the inhibition of polo-like kinases is concerned.

Another variation of the first embodiment of the present invention are compounds of the general formula I in claim 2, as described in claim 1, in which the following mean
R³ represents K, L or M,
X represents halogen, hydroxyl or the group —OR⁶, —NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
L represents the group —O—R⁷, —O—(CH₂)ₙ—(CO)—NH—R⁸, or —O—(CH₂)ₙ—(CO)—O—R⁸,
R⁹ represents C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can optionally be interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹²—(SO₂)—R²—NR¹²R¹³ or with C₁-C₃-alkyl optionally mono- or polysubstituted, identically or differently, with halogen, hydroxyl, C₁-C₃-alkylthiol or phenyl, wherein the aryl itself can be mono- or polysubstituted, identically or differently, with halogen or C₁-C₃-alkoxyl,
R¹⁶ represents hydrogen or C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or C₁-C₄-alkyl mono- or polysubstituted, identically or differently, with C₁-C₄-alkoxyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or represents methyl optionally mono- or polysubstituted, identically or differently, with C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, C₁-C₃-alkyl or C₁-C₃-alkoxyl, or C₁-C₄-alkyl mono- or polysubstituted, identically or differently, with C₂-C₁₀-heterocycloalkyl, or C₂-C₄-alkyl mono- or polysubstituted, identically or differently, with C₁-C₄-alkoxy-C₁-C₄-alkoxyl, wherein the heterocycloalkyl contains at least one atom in the ring, identically or differently, from the following group, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxyl, and
as well as their solvates, hydrates, diastereomers, enantiomers and salts.

Another object according to this first embodiment of the present invention are also compounds of the general formula I in claim 3, as described in claim 1 or 2, in which the following mean
R⁷ represents C₁-C₃-alkyl optionally mono- or polysubstituted, identically or differently, with —NR¹²R¹³ or C₂-C₁₀-heterocycloalkyl, wherein the heterocycloalkyl contains at least one atom, identically or differently, from the group nitrogen, oxygen or sulfur and can be optionally interrupted in the ring by one or several —(CO)— or —SO₂— groups and optionally one or several double bonds can be contained in the ring,
R⁹ represents C₁-C₅-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹², wherein the heterocycloalkyl contains at least one atom in the ring, identically or differently, from the group nitrogen, oxygen or sulfur and can optionally be interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, phenyl, which themselves can be optionally mono- or polysubstituted, identically or differently, with halogen or C₁-C₃-alkoxyl, or with the group —(CO)—R⁵, —(CO)—O—R¹², —(SO₂)—R¹⁴—NR¹²R¹³ or with C₁-C₃-alkyl optionally mono- or polysubstituted, identically or differently, with halogen, hydroxyl, C₁-C₃-alkylthiol or phenyl,
R¹⁰ and R¹¹ independently represent C₁-C₅-alkyl, C₂-C₁₀-heterocycloalkyl, aryl, or heteroaryl optionally mono- or polysubstituted, identically or differently, with halogen, C₁-C₃-alkyl, and C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
R¹⁴ represents C₁-C₃-alkyl or phenyl and
n is 1-4,
as well as their solvates, hydrates, diastereomers, enantiomers and salts.

Another variation of the first embodiment of the present invention are compounds of the general formula I in claim 4, as described in any of claims 1 through 3, in which the following mean
R¹ represents methyl, ethyl, isopropyl or cyclopropyl optionally mono- or polysubstituted, identically or differently, with halogen,
R² represents methyl, ethyl, allyl, propargyl optionally mono- or polysubstituted, identically or differently, with cyanogen, cyclopropyl, ethinyl or halogen or for hydroxyethyl at least monosubstituted with methyl,
X represents halogen, hydroxyl or the group —OR⁶, —NR¹⁰R¹¹ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves can be optionally mono- or polysubstituted, identically or differently, with halogen, hydroxyl, or phenyl, which themselves can be optionally mono- or polysubstituted, identically or differently, with halogen or C₁-C₃-alkoxyl or with the group —(CO)—R⁵, —NR¹²R¹³ or with C₁-C₃-alkyl optionally mono- or polysubstituted, identically or differently, with cyanogen, halogen, hydroxyl or C₁-C₃-alkylthiol,
R⁴ represents hydrogen or halogen or methyl optionally mono- or polysubstituted, identically or differently, with halogen,
R⁵ represents methyl, ethyl, tert.-butyl, phenyl or —NH₂,
R⁶ represents —SO₂-methyl,
R⁷ represents C₁-C₃-alkyl optionally mono- or polysubstituted, identically or differently, with —N(C₁-C₃-alkyl)₂, pyrrolidinyl, morpholinyl or piperidinyl,
R⁸ represents methyl, ethyl, allyl or propargyl optionally mono- or polysubstituted, identically or differently, with cyanogen, cyclopropyl or halogen,
R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, cyanogen, cyclopropyl, halogen, hydroxyl or with the group —NR¹⁰R¹¹, —O—(CO)—R⁵, —(SO₂)—R¹⁴ or —O—(SO₂)—C₁-C₃-alkyl, wherein pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves can be optionally mono- or polysubstituted, identically or differently, with halogen, hydroxyl, phenyl or C₁-C₃-alkoxyl, or with the group —(CO)—R⁵, —(CO)—O—R⁵, —(SO₂)—R¹⁴, —N(CH₃)₂ or with methyl or ethyl optionally mono- or polysubstituted, identically or differently, with halogen, hydroxyl, methylthiol or phenyl,
R¹⁰ and R¹¹ independently represent C₁-C₅-alkyl, pyrrolidinyl, phenyl or pyridinyl optionally mono- or polysubstituted, identically or differently, with halogen, C₁-C₃-alkyl or C₁-C₃-alkoxyl,
R¹² and R¹³ independently represent hydrogen or methyl, ethyl, or isopropyl,
R¹⁴ represents C₁-C₄-alkyl or phenyl and
n is 1 or 2,
as well as their solvates, hydrates, diastereomers, enantiomers and salts.

Another object of the present invention according to this embodiment are also compounds of the general formula I in claim 5, as described in any of claims 1 through 4, in which the following mean
U represents —CH═, —CF═, —C(CH₃)═OR—N═,
R¹ represents methyl, ethyl, isopropyl or cyclopropyl optionally mono- or polysubstituted, identically or differently, with fluorine,
R² represents methyl, ethyl, allyl, propargyl optionally mono- or polysubstituted, identically or differently, with cyanogen, cyclopropyl, ethinyl or fluorine or
   for hydroxyethyl at least monosubstituted with methyl,
K represents methyl, ethyl or ethenyl optionally mono- or polysubstituted, identically or differently, with X,
X represents halogen, hydroxyl or the group —O—SO₂-methyl or pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently, with halogen, hydroxyl, phenyl or with methyl optionally mono- or polysubstituted, identically or differently, with halogen,
L represents the group —O—R⁷, —O—(CH₂)—(CO)—NH—R⁸ or —O—(CH₂)—(CO)—O—R⁸,
M represents the group —NH—R⁹, —NH—(CO)—R¹⁶, —NH—(CO)—O—R⁹ or —N(CH₃)—(CO)—R¹⁶,
R⁷ represents ethyl optionally mono- or polysubstituted, identically or differently, with —N(C₁-C₃-ALKYL)₂, pyrrolidinyl, morpholinyl or piperidinyl,
R⁸ represents methyl, ethyl, allyl or propargyl optionally mono- or polysubstituted, identically or differently, with cyanogen, cyclopropyl or fluorine and
R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, morpholinyl, thiomorpholinyl, cyanogen, cyclopropyl, halogen, hydroxyl or with the group —N(C₁-C₃-alkyl)₂, —O—(CO)—(C₁-C₃-alkyl) or —O—(SO₂)—C₁-C₃-alkyl, wherein pyrrolidinyl, piperidinyl, piperazinyl, morpholinyl, thiomorpholinyl themselves can be optionally mono- or polysubstituted, identically or differently, with halogen or with the group —(CO)—C₁-C₄-alkyl, —(CO)—O—C₁-C₄-alkyl, —(SO₂)—C₁-C₃-alkyl, —(SO₂)-phenyl, —N(C₁-C₃-alkyl)₂ or with methyl or ethyl optionally mono- or polysubstituted, identically or differently, with halogen, hydroxyl or C₁-C₃-alkylthiol,
as well as their solvates, hydrates, diastereomers, enantiomers and salts.

Another object of the invention according to this embodiment are also compounds of the general formula I in claim 6, as described in any of claims 1 through 5, in which
R¹ represents ethyl,
X represents iodine, hydroxyl or the group —O—SO₂-methyl or pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently, with halogen, hydroxyl, phenyl or with methyl optionally mono- or polysubstituted, identically or differently, with halogen,
R⁷ represents ethyl optionally mono- or polysubstituted, identically or differently, with —N(CH₃)₂, pyrrolidinyl, morpholinyl or piperidinyl,
R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with methoxyl, ethoxyl, butoxy-ethoxyl, methoxy-ethoxyl, pyrrolidinyl, piperidinyl, piperazinyl, morpholinyl, thiomorpholinyl, cyanogen, cyclopropyl, chlorine, fluorine, hydroxyl or with the group —N(CH₃)₂, —N(CH₃)(C₂H₅), —O—(CO)—(CH₃) OR—O—(SO₂)-methyl, wherein pyrrolidinyl, piperidinyl, piperazinyl, morpholinyl, thiomorpholinyl themselves can be optionally mono- or polysubstituted, identically or differently, with fluorine, or with the group —(CO)—CH₃, —(CO)—C₂H₅, —(CO)—C(CH₃)₃, —(CO)—O—C(CH₃)₃, —(SO₂)—CH₃, —(SO₂)-phenyl, —N(CH₃)₂ or with methyl or ethyl optionally mono- or polysubstituted, identically or differently, with fluorine, hydroxyl or methylthiol,
as well as their solvates, hydrates, diastereomers, enantiomers and salts.

Another object of the first embodiment of this invention are also compounds of the general formula I in claim 7, as described in any of claims 1 through 6, in which the following means
R¹⁶ represents C₁-C₄-alkyl optionally mono- or polysubstituted, identically or differently, with C₁-C₄-alkoxyl, cyanogen, cyclopropyl, halogen, hydroxyl or with the group —NR¹⁰R¹¹, —O—(CO)—R⁵, —(SO₂)—R¹⁴, or —O—(SO₂)—R¹⁴ or methyl optionally mono- or polysubstituted, identically or differently, with C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen or C₁-C₃-alkoxyl,
as well as their solvates, hydrates, diastereomers, enantiomers and salts.

Another object of the first embodiment of this invention are also compounds of the general formula I in claim 8, as described in claim 7, in which the following means
R¹⁶ represents C₁-C₄-alkyl optionally mono- or polysubstituted, identically or differently, with the group —NR¹⁰R¹¹, or methyl optionally mono- or polysubstituted, identically or differently, with C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, or —

SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen or C₁-C₃-alkoxyl,
as well as their solvates, hydrates, diastereomers, enantiomers and salts.
Another object of the first embodiment of the present invention are compounds of the general formula I, as described in any of claims 1 through 8, in which K represents C₁-C₃-alkyl or C₂-C₄-alkenyl optionally mono- or polysubstituted, identically or differently, with X.
Another object of the first embodiment of the present invention are compounds of the general formula I, as described in any of claims 1 through 8, in which K represents C₁-C₃-alkyl or C₂-C₄-alkenyl mono- or polysubstituted, identically or differently, with X.
Another preferred object of the first embodiment of the present invention are compounds of the general formula I, as described in any of claims 1 through 8, in which K stands methyl, ethyl or ethenyl optionally mono- or polysubstituted, identically or differently, with X.
Another object of the first embodiment of the present invention are compounds of the general formula I, as described in any of claims 1 through 8, in which L represents the group —O—R⁷, —O—(CH₂)ₙ—(CO)—NH—R⁸, —O—(CH₂)ₙ—(CO)—R¹⁵ or —O—(CH₂)ₙ—(CO)—O—R⁸.
Another object of the first embodiment of the present invention are compounds of the general formula I, as described in any of claims 1 through 8, in which L represents the group —O—R⁷, —O—(CH₂)ₙ—(CO)—NH—R⁸ or —O—(CH₂)ₙ—(CO)—O—R⁸.
Another preferred object of the first embodiment of the present invention are compounds of the general formula I, as described in any of claims 1 through 8, in which L represents the group —O—R⁷, —O—(CH₂)—(CO)—NH—R⁸ or —O—(CH₂)—(CO)—O—R⁸.
Another object of this first embodiment of the present invention are compounds of the general formula I, as described in any of claims 1 through 8, in which R⁵ represents C₁-C₄-alkyl, phenyl or —NR¹²R¹³.
Another preferred object of this first embodiment of the present invention are compounds of the general formula I, as described in any of claims 1 through 8, in which R⁵ represents methyl, ethyl, tert.-butyl, phenyl or —NH₂.
Another object of the first embodiment of the present invention are compounds of the general formula I, as described in claims 1 through 8, in which R¹⁶ represents hydrogen or C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴, or —O—(SO₂)—R¹⁴ or C₁-C₄-alkyl mono- or polysubstituted, identically or differently, with C₁-C₄-alkoxyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or represents methyl optionally mono- or polysubstituted, identically or differently, with C₂-C₁₀-heterocycloalkyl or heteroaryl, but preferably without heteroaryl, wherein the heterocycloalkyl contains at least one atom in the ring, identically or differently, from the group nitrogen, oxygen or sulfur and can be optionally interrupted in the ring by one or several —(CO)—, —(C═S)—, but preferably without —(C═S)—, or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, C₁-C₃-alkyl, but preferably without C₁-C₃-alkyl, or with C₁-C₃-alkoxyl,
   or represents C₁-C₄-alkyl mono- or polysubstituted, identically or differently, with C₂-C₁₀-heterocycloalkyl, or represents C₂-C₄-alkyl mono- or polysubstituted, identically or differently, with C₁-C₄-alkoxy-C₁-C₄-alkoxyl, wherein the heterocycloalkyl contains at least one atom in the ring, identically or differently, from the following group, oxygen or sulfur and can be optionally interrupted in the ring by one or several —(CO)—, —(C═S)—, but preferably without —(C═S)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹²—(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl, but preferably without C₁-C₃-alkyl, or with C₁-C₃-alkoxy.
Another preferred object of the first embodiment of the present invention is compounds of the general formula I, as described in any of claims 1 through 8, in which R¹⁶ represents C₁-C₄-alkyl mono- or polysubstituted, identically or differently, with C₁-C₄-alkoxyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴ or methyl optionally mono- or polysubstituted, identically or differently, with C₂-C₁₀-heterocycloalkyl or heteroaryl, but preferably without heteroaryl, wherein the heterocycloalkyl contains at least one atom in the ring, identically or differently, from the group nitrogen, oxygen or sulfur and can be optionally interrupted in the ring by one or several —(CO)—, —(C═S)—, but preferably without —(C═S)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, with C₁-C₃-alkyl, but preferably without C₁-C₃-alkyl, or with C₁-C₃-alkoxy.
Another preferred object of the first embodiment of the present invention are compounds of the general formula I, as described in any of claims 1 through 8, in which R¹⁶ represents C₁-C₄-alkyl mono- or polysubstituted, identically or differently, with the group —NR¹⁰R¹¹ or methyl optionally mono- or polysubstituted, identically or differently, with C₂-C₁₀-heterocycloalkyl or heteroaryl, but preferably without heteroaryl, wherein the heterocycloalkyl contains at least one atom in the ring, identically or differently, from the group nitrogen, oxygen or sulfur and can be optionally interrupted in the ring by one or several —(CO)—, —(C═S)—, but preferably without —(C═S)—, or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, with C₁-C₃-alkyl, but preferably without C₁-C₃-alkyl or C₁-C₃-alkoxy.
Another preferred object of the first embodiment of the present invention are compounds of the general formula I, as described in any of claims 1 through 8, in which R¹⁶ represents methyl optionally mono- or polysubstituted, identically or differently, with the group —NR¹⁰R¹¹, C₂-C₁₀-heterocycloalkyl, imidazolyl or benzimidazolyl, but preferably without imidazolyl or benzimidazolyl, wherein the heterocycloalkyl contains at least one atom in the ring, identically or differently, from the group nitrogen, oxygen or sulfur and can be optionally interrupted in the ring by one or several —(CO)—, —(C═S)—, but preferably without —(C═S)— or —SO₂— groups and optionally one or several double bonds can be contained in the ring and the ring itself can be optionally mono- or polysubstituted, identically or differently, with halogen, cyanogen, hydroxyl, phenyl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the phenyl itself can be optionally mono- or polysubstituted, identically or differently with halogen, with C₁-C₃-alkyl, but preferably without C₁-C₃-alkyl or with C₁-C₃-alkoxy.
Another preferred object of the first embodiment of the present invention are compounds of the general formula I, as described in any of claims 1 through 8, in which R¹⁶ represents methyl optionally mono- or polysubstituted, identically or differently, with pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, imidazolyl or benzimidazolyl, but preferably without imidazolyl or benzimidazolyl, or with the group —NR¹⁰R¹¹, wherein pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves can be optionally mono- or polysubstituted, identically or differently, with halogen, hydroxyl, phenyl or with the group —(CO)—R⁵, —(CO)—O—R⁵, —(SO₂)—R¹⁴, —N(CH₃)₂ or with methyl or ethyl optionally mono- or polysubstituted, identically or differently, with halogen, hydroxyl, methylthiol or phenyl, wherein the phenyl itself can be optionally mono- or polysubstituted, identically or differently with halogen, with C₁-C₃-alkyl, but preferably without C₁-C₃-alkyl or with C₁-C₃-alkoxy.

### SECOND EMBODIMENT OF THE PRESENT INVENTION

In a second embodiment of the present invention it was found that compounds of the general formula I in claim 9, as described in claim 1, in which
K represents C₁-C₃-alkyl mono- or polysubstituted, identically or differently, with P or C₂-C₄-alkenyl mono- or polysubstituted, identically or differently, with X,
P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring of the C₂-C₅-heterocycloalkyl itself is mono- or polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol, wherein the aryl itself can be optionally mono- or polysubstituted, identically or differently, with cyanogen, halogen or C₁-C₃-alkoxyl, and the ring of the C₆-C₁₀-heterocycloalkyl itself can be mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkyl, wherein the aryl itself can be optionally mono- or polysubstituted, identically or differently, with cyanogen, halogen or C₁-C₃-alkoxyl,
L represents the group —O—R⁷, —O—(CH₂)ₙ—(CO)—NH—R³, —O—(CH₂)ₙ—(CO)—R¹⁵ or —O—(CH₂)ₙ—(CO)—O—R⁸,
R⁷ represents C₁-C₃-alkyl optionally mono- or polysubstituted, identically or differently, with C₆-C₁₀-heterocycloalkyl, wherein the C₆-C₁₀-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen, or for C₁-C₃-Alkyl mono- or polysubstituted, identically or differently, with C₂-C₅-Heterocycloalkyl, wherein the C₂-C₅-heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be mono- or polysubstituted, identically or differently, with halogen, aryl or with C₁-C₃-alkyl mono- or polysubstituted, identically or differently, with halogen,
R¹⁶ represents hydrogen, C₂-C₄-alkenyl, cyclopropyl, C₂-C₅-heterocycloalkyl, C₆-C₁₀heterocycloalkyl or a methyl substituted with heteroaryl or C₁-C₄-alkenyl, C₂-C₄-alkenyl, cyclopropyl, C₂-C₅-heterocycloalkyl or C₆-C₁₀heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁹R¹⁹, —O—(CO)—R⁵, —(SO₂)—R¹⁴ or —O—(SO₂)—R¹⁴, wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring of the C₂-C₅-heterocycloalkyl itself can be optionally mono- or polysubstituted, identically or differently, with halogen, cyanogen, hydroxyl, aryl or with the group —(CO)—R⁵, —(CO)—O—R¹², —(SO₂)—R¹⁴ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, C₁-C₃-alkyl or C₁-C₃-alkoxyl, and the ring of the C₆-C₁₀-heterocycloalkyl can be optionally mono- or polysubstituted, identically or differently, with halogen, cyanogen, hydroxyl, aryl or with the group —(CO)—R⁵—(CO)—O—R¹²—(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen or C₁-C₃-alkoxyl,
R¹⁷ represents C₁-C₃-alkyl mono- or polysubstituted, identically or differently, with halogen or cyanogen or C₃-C₄-alkenyl or C₃-C₄-alkinyl optionally mono- or polysubstituted, identically or differently, with halogen, cyclopropyl or cyanogen,
R¹⁸ and R¹⁹ independently represent C₁-C₅-alkyl, C₂-C₁₀-heterocycloalkyl, aryl, —(CH₂)ₙ-aryl or heteroaryl optionally mono- or polysubstituted, identically or differently, with halogen, C₁-C₃-alkyl or C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring, wherein
   either R¹⁸ or R¹⁹ represents a C₂-C₁₀-heterocycloalkyl, —(CH₂)ₙ-aryl, or a heteroaryl, or a C₂-C₁₀-heterocycloalkyl, —(CH₂)ₙ-aryl or heteroaryl optionally mono- or polysubstituted, identically or differently, with halogen, C₁-C₃-alkyl, C₁-C₃-alkoxyl, or a C₁-C₅-alkyl mono- or polysubstituted, identically or differently, with C₁-C₃-alkoxyl, or an aryl mono- or polysubstituted, identically or differently, with C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring,
as well as their solvates, hydrates, diastereomers, enantiomers and salts solve the task of the present invention.

Another variation of the second embodiment of the present invention are compounds of the general formula I in claim 10, as described in claim 9,

in which
T, T² and T3 independently represent —CH═ or —N═
R³ is K, L or M,
P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring of the C₂-C₅-heterocycloalkyl itself is mono- or polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol, wherein the aryl itself can be optionally mono- or polysubstituted, identically or differently, with cyanogen, halogen or C₁-C₃-alkoxyl, and the ring of the C₆-C₁₀-heterocycloalkyl itself can be optionally mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
L represents the group —O—R⁷, —O—(CH₂)ₙ—(CO)—NH—R¹⁷, or —O—(CH₂)ₙ—(CO)—R⁸,
R⁹ represents C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen or C₁-C₃-alkoxyl,
R¹⁶ represents hydrogen, C₂-C₄-alkenyl, cyclopropyl, C₂-C₅-heterocycloalkyl, C₆-C₁₀heterocycloalkyl or a methyl substituted with heteroaryl or C₁-C₄-alkenyl, C₂-C₄-alkenyl, cyclopropyl, C₂-C₅-heterocycloalkyl or C₆-C₁₀heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁸R¹⁹, —O—(CO)—R⁵, —(SO₂)—R¹⁴ or —O—(SO₂)—R¹⁴, wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring of the C₂-C₅-heterocycloalkyl itself can be optionally mono- or polysubstituted, identically or differently, with halogen, cyanogen, hydroxyl, aryl or with the group —(CO)—R⁵, —(CO)—O—R¹², —(SO₂)—R¹⁴ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, C₁-C₃-alkyl or C₁-C₃-alkoxyl, and the ring of the C₆-C₁₀-heterocycloalkyl can be optionally mono- or polysubstituted, identically or differently, with halogen, cyanogen, hydroxyl, aryl or with the group —(CO)—R⁵, —(CO)—O—R¹², —(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen or C₁-C₃-alkoxyl,
as well as their solvates, hydrates, diastereomers, enantiomers and salts.

Another variation of the second embodiment of the present invention are compounds of the general formula I in claim 22, as described in claim 9, in which
P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring of the C₂-C₅-heterocycloalkyl itself is polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol, wherein the aryl itself can be optionally mono- or polysubstituted, identically or differently, with cyanogen, halogen or C₁-C₃-alkoxyl, and the ring of the C₆-C₁₀-heterocycloalkyl itself can be optionally mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
R⁵ represents C₁-C₄-alkyl or phenyl,
R⁷ represents C₁-C₃-alkyl optionally mono- or polysubstituted, identically or differently, with C₆-C₁₀-heterocycloalkyl, wherein the C₆-C₁₀-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen, or C₁-C₃-alkyl mono- or polysubstituted, identically or differently, with C₂-C₅-heterocycloalkyl, wherein the C₂-C₅-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the C₂-C₅-heterocycloalkyl ring itself can be polysubstituted, identically or differently, with halogen or aryl or with C₁-C₃-alkyl mono- or polysubstituted, identically or differently, with halogen,
R¹⁶ represents hydrogen, C₂-C₄-alkenyl, cyclopropyl, C₂-C₅-heterocycloalkyl, C₆-C₁₀-heterocycloalkyl or a methyl substituted with heteroaryl or C₁-C₄-alkenyl, C₂-C₄-alkenyl, cyclopropyl, C₂-C₅-heterocycloalkyl or C₆-C₁₀-heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁸R¹⁹, —O—(CO)—R⁵—(SO₂)—R¹⁴, or —O (SO₂)—R¹⁴, wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained in the ring and the ring of the C₂-C₅-heterocycloalkyl itself can be polysubstituted, identically or differently, with halogen, cyanogen, hydroxyl, aryl or with the group —(CO)—R⁵, —(CO)—O—R¹², —(SO₂)—R¹⁴, wherein the aryl itself can be optionally mono- or polysubstituted, identically or differently with halogen, C₁-C₃-alkyl or C₁-C₃-alkoxy or the ring of the C₂-C₅-heterocycloalkyl is monosubstituted with halogen, cyanogen, hydroxyl, aryl, wherein in this case the aryl itself is mono- or polysubstituted, identically or differently, with halogen, C₁-C₃-alkyl or C₁-C₃-alkoxy or the ring of the C₂-C₅-heterocycloalkyl is mono- or polysubstituted, identically or differently, with halogen, C₁-C₃-alkyl or phenyl, and the ring of the C₆-C₁₀-heterocycloalkyl can be optionally mono- or polysubstituted, identically or differently, with halogen, cyanogen, hydroxyl, aryl or with the group —(CO)—R⁵, —(CO)—O—R¹²—(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxy.
as well as their solvates, hydrates, diastereomers, enantiomers and salts.

Another variation of the second embodiment of the present invention are compounds of the general formula I in claim 23, as described in claim 10, in which
P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring of the C₂-C₅-heterocycloalkyl itself is polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol, wherein the aryl itself can be optionally mono- or polysubstituted, identically or differently, with cyanogen, halogen or C₁-C₃-alkoxyl, and the ring of the C₆-C₁₀-heterocycloalkyl itself can be optionally mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
R⁵ represents C₁-C₄-alkyl or phenyl,
R⁷ represents C₁-C₃-alkyl optionally mono- or polysubstituted, identically or differently, with C₆-C₁₀-heterocycloalkyl, wherein the C₆-C₁₀-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen, or
   C₁-C₃-alkyl mono- or polysubstituted, identically or differently, with C₂-C₅-heterocycloalkyl, wherein the C₂-C₅-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the C₂-C₅-heterocycloalkyl ring itself can be polysubstituted, identically or differently, with halogen or aryl or with C₁-C₃-alkyl mono- or polysubstituted, identically or differently, with halogen,
R¹⁶ represents hydrogen, C₂-C₄-alkenyl, cyclopropyl, C₂-C₅-heterocycloalkyl, C₆-C₁₀-heterocycloalkyl or C₁-C₄-alkenyl, C₂-C₄-alkenyl, cyclopropyl, C₂-C₅-heterocycloalkyl or C₆-C₁₀heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁸R¹⁹, —O—(CO)—R⁵, —(SO₂)—R¹⁴ or —O—(SO₂)—R¹⁴, wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring of the C₂-C₅-heterocycloalkyl itself is polysubstituted, identically or differently, with halogen, cyanogen, hydroxyl, aryl or with the group —(CO)—R⁵, —(CO)—O—R¹², —(SO₂)—R¹⁴, wherein the aryl itself can be optionally mono- or polysubstituted, identically or differently with halogen or C₁-C₃-alkoxy or the ring of the C₂-C₅-heterocycloalkyl is monosubstituted with —(CO)—O—R¹², —(CO)—R⁵ or aryl, wherein in this case the aryl itself is mono- or polysubstituted, identically or differently, with halogen or C₁-C₃-alkoxyl or the ring of the C₂-C₅-heterocycloalkyl is mono- or polysubstituted, identically or differently, with halogen, C₁-C₃-alkylthiol or phenyl, and the ring of the C₆-C₁₀-heterocycloalkyl can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxy,
as well as their solvates, hydrates, diastereomers, enantiomers and salts.
Another variation of the second embodiment of the present invention are compounds of the general formula I in claim 24, as described in any of claims 9, 10, 22 or 23, in which P represents the group —OR⁶, —NR¹⁸R¹⁹ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are mono- or polysubstituted, identically or differently with halogen, hydroxyl, phenyl, which themselves can be optionally mono- or polysubstituted, identically or differently, with halogen or C₁-C₃-alkoxyl, or with C₁-C₃-alkyl mono- or polysubstituted, identically or differently, with the group —(CO)—R⁵ or mono- or polysubstituted, identically or differently, with cyanogen, halogen or C₁-C₃-alkylthiol,
   as well as their solvates, hydrates, diastereomers, enantiomers and salts.

Another variation of the second embodiment of the present invention are compounds of the general formula I in claim 25, as described in any of claims 9, 10, 22, 23 or 24, in which R¹⁸ and R¹⁹ independently represent C₁-C₅-alkyl, pyrrolidinyl, phenyl or pyridinyl optionally mono- or polysubstituted, identically or differently, with halogen, C₁-C₃-alkyl or C₁-C₃-alkoxyl, wherein either R¹⁸ and R¹⁹ represent pyrrolidinyl or pyridinyl or a pyrrolidinyl or pyridinyl mono- or polysubstituted, identically or differently with halogen, C₁-C₃-alkyl or C₁-C₃-alkoxyl.
Another object of the second embodiment of the present invention are compounds of the general formula I, as described in any of claims 9, 10, 22, 23, 24 or 25, in which K represents C₁-C₃-alkyl mono- or polysubstituted, identically or differently, with P or C₂-C₄-alkenyl mono- or polysubstituted, identically or differently, with X,
Another preferred object of the second embodiment of the present invention are compounds of the general formula I, as described in any of claims 9, 10, 22, 23, 24 or 25, in which K represents C₁-C₃-alkyl mono- or polysubstituted, identically or differently, with P,
Another object of the second embodiment of the present invention is compounds of the general formula I, as described in any of claims 9, 10, 22, 23, 24 or 25, in which P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, —(C═S)—, but preferably without —(C═S)—, or —SO₂— groups and optionally one or several double bonds can be contained in the ring and the ring of the C₂-C₅-heterocycloalkyl itself is mono- or polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol, wherein the aryl itself can be optionally mono- or polysubstituted, identically or differently, with cyanogen, halogen or C₁-C₃-alkoxyl, and the ring of the C₆-C₁₀-heterocycloalkyl itself can be mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkyl, wherein the aryl itself can be optionally mono- or polysubstituted, identically or differently, with cyanogen, halogen or C₁-C₃-alkoxyl,
Another object of the second embodiment of the present invention are compounds of the general formula I, as described in any of claims 9, 10, 22, 23, 24 or 25, in which P represents the group —OR⁶, —NR¹⁸R¹⁹, C₂-C₅-heterocycloalkyl or C₆-C₁₀ heterocycloalkyl, wherein the C₂-C₅-heterocycloalkyl and the C₆-C₁₀ heterocycloalkyl contain at least one atom in the ring, identically or differently, from the group nitrogen, oxygen or sulfur and can be optionally interrupted in the ring by one or several —(CO)—, —(C═S)—, but preferably without —(C═S)—, or —SO₂— groups and optionally one or several double bonds can be contained in the ring and the ring of the C₂-C₅-heterocycloalkyl itself is polysubstituted, identically or differently, with cyanogen, halogen, hydroxyl, aryl or with the group —(CO)—R⁵, or with C₁-C₃-alkyl mono- or polysubstituted, identically or differently, with halogen or C₁-C₃-alkylthiol or the ring of the C₂-C₁₅-heterocycloalkyl itself can be monosubstituted with the group —(CO)—R⁵, wherein the aryl itself can be optionally mono- or polysubstituted, identically or differently, with cyanogen, halogen or C₁-C₃-alkoxyl, and the ring of the C₆-C₁₀-heterocycloalkyl itself can be mono- or polysubstituted, identically or differently, with cyanogen, halogen, hydroxyl, aryl or with the group —(CO)—R⁵, —NR¹²R¹³ or optionally with C₁-C₃-alkyl mono- or polysubstituted, identically or differently, with halogen, hydroxyl or C₁-C₃-alkyl, wherein the aryl itself can be optionally mono- or polysubstituted, identically or differently, with cyanogen, halogen or C₁-C₃-alkoxy.
Another object of the second embodiment of the present invention are compounds of the general formula I, as described in any of claims 9, 10, 22, 23, 24 or 25, in which P represents the group —OR⁶, —NR¹⁸R¹⁹ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are mono- or polysubstituted, identically or differently with halogen, hydroxyl, phenyl, which themselves can be optionally mono- or polysubstituted, identically or differently, with halogen or C₁-C₃-alkoxyl, or with C₁-C₃-alkyl mono- or polysubstituted, identically or differently, with the group —(CO)—R⁵ or mono- or polysubstituted, identically or differently, with cyanogen, halogen or C₁-C₃-alkylthiol.
   In a preferred variation, the pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are polysubstituted identically or differently with cyanogen, halogen, hydroxyl, aryl or with the group —(CO)—R⁵ or with C₁-C₃-alkyl mono- or polysubstituted, identically or differently, with halogen or C₁-C₃-alkylthiol or monosubstituted with the group —(CO)—R⁵, wherein the aryl itself can be optionally mono- or polysubstituted, identically or differently, with cyanogen, halogen or C₁-C₃-alkoxy.
Another object of the second embodiment of the present invention are compounds of the general formula I, as described in any of claims 9, 10, 22, 23, 24 or 25, in which L represents the group —O—R⁷—O—(CH₂)ₙ—(CO)—NH—R¹⁷, —O—(CH₂)ₙ—(CO)—R¹⁵ or —O—(CH₂)ₙ—(CO)—O—R³. Preferably, L represents the group —O—R⁷—O—(CH₂)ₙ—(CO)—NH—R¹⁷ or —O—(CH₂)ₙ—(CO)—O—R⁸.
Another object of the second embodiment of the present invention are compounds of the general formula I, as described in any of claims 9, 10, 22, 23, 24 or 25, in which R⁵ represents C₁-C₄-alkyl, phenyl or —NR¹²R¹³.
Another object of this second embodiment of the present invention are compounds of the general formula I, as described in any of claims 9, 10, 22, 23, 24 or 25, in which R⁵ represents C₁-C₄-alkyl or phenyl.
Another preferred object of this second embodiment of the present invention are compounds of the general formula I, as described in any of claims 9, 10, 22, 23, 24 or 25, in which R⁵ stands methyl, ethyl, tert.-butyl, or phenyl.
Another object of the second embodiment of the present invention are compounds of the general formula I, as described in any of claims 9, 10, 22, 23, 24 or 25, in which R⁷ represents C₁-C₃-alkyl optionally mono- or polysubstituted, identically or differently, with C₆-C₁₀-heterocycloalkyl, wherein the C₆-C₁₀-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen, or
   C₁-C₃-alkyl mono- or polysubstituted, identically or differently, with C₂-C₅-heterocycloalkyl, wherein the C₂-C₅-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself is mono- or polysubstituted, identically or differently, with halogen or aryl or with C₁-C₃-alkyl mono- or polysubstituted, identically or differently, with halogen.
Another object of the second embodiment of the present invention are compounds of the general formula I, as described in any of claims 9, 10, 22, 23, 24 or 25, in which R⁷ represents C₁-C₃-alkyl optionally mono- or polysubstituted, identically or differently, with C₆-C₁₀-heterocycloalkyl, wherein the C₆-C₁₀-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen, or
   C₁-C₃-alkyl mono- or polysubstituted, identically or differently, with C₂-C₅-heterocycloalkyl, wherein the C₂-C₅-heterocycloalkyl contains at least one atom in the ring, identically or differently, from the group nitrogen, oxygen or sulfur and can be optionally interrupted in the ring by one or several —(CO)—, or —SO₂— groups and optionally one or several double bonds can be contained in the ring and the C₂-C₅-heterocycloalkyl ring itself is mono- or polysubstituted, identically or differently, with halogen or aryl or with C₁-C₃-alkyl mono- or polysubstituted, identically or differently, with halogen.
Another object of the second embodiment of the present invention are compounds of the general formula I, as described in any of claims 9, 10, 22, 23, 24 or 25, in which R¹⁶ represents hydrogen, C₂-C₄-alkenyl, cyclopropyl, C₂-C₅-heterocycloalkyl, C₆-C₁₀-heterocycloalkyl or a methyl substituted with heteroaryl, but preferably a methyl not substituted with heteroaryl or C₁-C₄-alkenyl, C₂-C₄-alkenyl, cyclopropyl, C₂-C₅-heterocycloalkyl or C₆-C₁₀-heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁸R¹⁹, —O—(CO)—R⁵, —(SO₂)—R¹⁴ or —O—(SO₂)—R¹⁴ wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, —(C═S)— but preferably without —(C═S)— or with —SO₂— groups and optionally one or several double bonds can be contained in the ring and the ring of the C₂-C₅-heterocycloalkyl itself is mono- or polysubstituted, identically or differently, with halogen, cyanogen, hydroxyl, aryl or with the group —(CO)—R⁵, —(CO)—O—R¹², —(SO₂)—R¹⁴, or with C₁-C₃-alkyl mono- or polysubstituted, identically or differently, with halogen, with halogen, C₁-C₃-alkylthiol or phenyl, wherein the aryl itself can be optionally mono- or polysubstituted, identically or differently with halogen or C₁-C₃-alkoxy, but preferably without C₁-C₃-alkyl, or with C₁-C₃-alkoxyl, and the ring of the C₆-C₁₀-heterocycloalkyl can be optionally mono- or polysubstituted, identically or differently, with halogen, cyanogen, hydroxyl, aryl or with the group —(CO)—R⁵, —(CO)—O—R¹², —(SO₂)—R¹⁴—NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxyl,
Another preferred object of the second embodiment of the present invention are compounds of the general formula I, as described in any of claims 9, 10, 22, 23, 24 or 25, in which R¹⁶ represents hydrogen, but preferably not hydrogen, C₂-C₄-alkenyl, cyclopropyl, C₂-C₅-heterocycloalkyl, C₆-C₁₀-heterocycloalkyl or a methyl substituted with heteroaryl, but preferably for a methyl not substituted with heteroaryl, or C₁-C₄-alkenyl, C₂-C₄-alkenyl, cyclopropyl, C₂-C₅-heterocycloalkyl or C₆-C₁₀-heterocycloalkyl mono- or polysubstituted, identically or differently, with C₁-C₄-alkoxyl, C₂-C₅-heterocycloalkyl, C₆-C₁₀-heterocycloalkyl, cyanogen, cyclopropyl or with the group —NR¹⁸R¹⁹, —O—(CO)—R⁵, —(SO₂)—R¹⁴ or —O—(SO₂)—R¹⁴ wherein the C₂-C₅-heterocycloalkyl and the C₆-C₁₀-heterocycloalkyl contain at least one atom in the ring, identically or differently, from the group nitrogen, oxygen or sulfur and can be optionally interrupted in the ring by one or several —(CO)—, —(C═S)— but preferably without —(C═S)—, or with —SO₂— groups and optionally one or several double bonds can be contained in the ring and the ring of the C₂-C₅-heterocycloalkyl itself is polysubstituted, identically or differently, with halogen, cyanogen, hydroxyl, aryl or with the group —(CO)—R⁵, —(CO)—O—R¹², —(SO₂)—R¹⁴, wherein in this case the aryl can be optionally mono- or polysubstituted, identically or differently, with halogen, C₁-C₃-alkyl but preferably without C₁-C₃ alkyl, or with C₁-C₃-alkoxy or the ring of the C₂-C₅-heterocycloalkyl is monosubstituted with —(CO)—O—R¹², —(CO)—R⁵, or aryl, wherein in this case the aryl itself is mono- or polysubstituted, identically or differently, with halogen, C₁-C₃-alkyl, but preferably without C₁-C₃-alkyl, or with C₁-C₃-alkoxy or the ring of the of the C₂-C₅-heterocycloalkyl is substituted with C₁-C₃-alkyl mono- or polysubstituted, identically or differently, with halogen, C₁-C₃-alkylthiol or phenyl and the ring of the C₆-C₁₀-heterocycloalkyl can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxyl,
Another object of the second embodiment of the present invention are compounds of the general formula I, as described in any of claims 9, 10, 22, 23, 24 or 25, in which R¹⁷ represents C¹-C³-alkyl mono- or polysubstituted, identically or differently, with halogen or cyanogen or C³-C⁴-alkenyl or C³-C⁴-alkinyl optionally mono- or polysubstituted, identically or differently, with halogen, cyclopropyl or cyanogen.
Another object of the second embodiment of the present invention are compounds of the general formula I, as described in any of claims 9, 10, 22, 23, 24 or 25, in which R¹⁸ and R¹⁹ independently represent C₁-C₅-alkyl, C₂-C₁₀-heterocycloalkyl, aryl, —(C₂)ₙ-aryl or heteroaryl optionally mono- or polysubstituted, identically or differently, with halogen, C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring, wherein
   either R¹⁸ or R¹⁹ represents a C₂-C₁₀-heterocycloalkyl, —(CH₂)ₙ-aryl, or a heteroaryl, or a C₂-C₁₀-heterocycloalkyl, —(CH₂)ₙ-aryl or heteroaryl optionally mono- or polysubstituted, identically or differently, with halogen, C₁-C₃-alkyl, C₁-C₃-alkoxyl, or a C₁-C₅-alkyl mono- or polysubstituted, identically or differently, with C₁-C₃-alkoxyl, or an aryl mono- or polysubstituted, identically or differently, with C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
Another object of the second embodiment of the present invention are compounds of the general formula I, as described in any of claims 9, 10, 22, 23, 24 or 25, in which R¹⁸ and R¹⁹ independently represent C₁-C₅-alkyl, C₂-C₁₀-heterocycloalkyl, aryl, —(C₂)ₙ-aryl or heteroaryl optionally mono- or polysubstituted, identically or differently, with halogen, C₁-C₃-alkyl or C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring, wherein either R¹⁸ or R¹⁹ represents a C₂-C₁₀-heterocycloalkyl or a heteroaryl, or C₂-C₁₀-heterocycloalkyl or heteroaryl optionally mono- or polysubstituted, identically or differently, with halogen, C₁-C₃-alkyl or C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring.
Another object of the second embodiment of the present invention are compounds of the general formula I, as described in any of claims 9, 10, 22, 23, 24 or 25, in which R¹⁸ and R¹⁹ independently represent C₁-C₅-alkyl, pyrrolidinyl, phenyl or pyridinyl optionally mono- or polysubstituted, identically or differently with halogen, C₁-C₃-alkyl or C₁-C₃-alkoxyl, wherein,
   either R¹⁸ or R¹⁹ represent pyrrolidinyl or pyridinyl or a pyrrolidinyl or pyridinyl mono- or polysubstituted, identically or differently, with halogen, C₁-C₃-alkyl or C₁-C₃-alkoxy.

### THIRD EMBODIMENT OF THE PRESENT INVENTION

The task of the present compound in the third embodiment is to furnish improved compounds, improved particularly in the inhibition of polo-like kinases as compared to prior art and/or to provide compounds that have better physicochemical properties as compared to compounds disclosed in prior art.

In a third embodiment of the present invention it was found in claim 11, as described in claim 1 and/or 2, that compounds of the general formula I, in which the following mean
R³ represents K or L,
K represents C₁-C₃-alkyl mono- or polysubstituted, identically or differently, with X, where by the C₁-C₃-alkyl can be mono- or polysubstituted, identically or differently with hydroxyl or halogen,
X represents NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, —(C═S)—, or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently with cyanogen, halogen or C₁-C₃-alkoxyl,
L represents the group —O—R⁷,
R⁷ represents C₁-C₃-alkyl mono- or polysubstituted, identically or differently, with —NR¹²R¹³ or C₂-C₁₀-heterocycloalkyl and the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, aryl or C₁-C₃-alkyl optionally mono- or polysubstituted, identically or differently, with halogen.
as well as their solvates, hydrates, diastereomers, enantiomers and salts solve the task of the present invention especially well.

Another variation of the third embodiment of the present invention are compounds of the general formula I in claim 12, as described in claim 11, in which
X represents —N(C₁-C₃-alkyl)₂ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are mono- or polysubstituted, identically or differently with halogen, hydroxyl, phenyl, which themselves can be optionally mono- or polysubstituted, identically or differently, with halogen or C₁-C₃-alkoxyl, or with C₁-C₃-alkyl mono- or polysubstituted, identically or differently, with the group —(CO)—R⁵ or mono- or polysubstituted, identically or differently, with cyanogen, halogen or C₁-C₃-alkylthiol.
R⁷ represents C₁-C₃-alkyl mono- or polysubstituted, identically or differently, with —N(C₁-C₃-alkyl)₂ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring.
as well as their solvates, hydrates, diastereomers, enantiomers and salts.
Another object of the third embodiment of the present invention are compounds of the general formula I, as described in any of claims 11 or 12, in which R³ represents K or L.
Another object of the third embodiment of the present invention are compounds of the general formula I, as described in any of claims 11 or 12, in which K represents C₁-C₃-alkyl mono- or polysubstituted, identically or differently, with X, wherein the C₁-C₃-alkyl can be optionally mono- or polysubstituted, identically or differently, with hydroxyl or halogen,
Another preferred object of the third embodiment of the present invention are compounds of the general formula I, as described in any of claims 11 or 12, in which K represents C₁-C₃-alkyl monosubstituted with X, wherein the C₁-C₃-alkyl can be optionally mono- or polysubstituted, identically or differently, with hydroxyl or halogen. Preferably, the C₁-C₃-alkyl is only substituted with X.
Another object of the third embodiment of the present invention are compounds of the general formula I, as described in any of claims 11 or 12, in which L represents the group —O—R⁷.
Another object of the third embodiment of the present invention are compounds of the general formula I, as described in any of claims 11 or 12, in which X represents NR¹⁰R¹¹ or C₂-C₁₀-heterocycloalkyl, wherein the heterocycloalkyl contains at least one atom in the ring, identically or differently, from the group nitrogen, oxygen or sulfur, and in a preferred variation contains at least one nitrogen, and can be optionally interrupted in the ring by one or several —(CO)—, —(C═S)—, or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently with cyanogen, halogen or C₁-C₃-alkoxyl,
Another preferred object of the third embodiment of the present invention are compounds of the general formula I, as described in any of claims 11 or 12, in which X represents azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are mono- or polysubstituted, identically or differently with halogen, hydroxyl, phenyl, which themselves can be optionally mono- or polysubstituted, identically or differently, with halogen or C₁-C₃-alkoxyl, or with C₁-C₃-alkyl mono- or polysubstituted, identically or differently, with the group —(CO)—R⁵ or mono- or polysubstituted, identically or differently, with cyanogen, halogen or C₁-C₃-alkylthiol.
   In a preferred variation, X represents unsubstituted azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl [sic].
Another preferred object of the third embodiment of the present invention are compounds of the general formula I, as described in any of claims 11 or 12, in which X represents pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently, with halogen, hydroxyl, phenyl or with methyl optionally mono- or polysubstituted, identically or differently, with halogen. In a preferred variation the pyrrolidinyl, morpholinyl, thiomorpholinyl, piperidinyl or octahydroisochinolinyl are not substituted.
Another object of the third embodiment of the present invention are compounds of the general formula I, as described in any of claims 11 or 12, in which R⁷ represents C₁-C₃-alkyl mono- or polysubstituted, identically or differently, with —NR¹²R¹³ or C₂-C₁₀-heterocycloalkyl and the heterocycloalkyl contains at least one atom in the ring, identically or differently, from the group nitrogen, oxygen or sulfur, and in a preferred variation contains at least one nitrogen, and can be optionally interrupted in the ring by one or several —(CO)— or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen.
Another object of the third embodiment of the present invention are compounds of the general formula I, as described in any of claims 11 or 12, in which R⁷ represents C₁-C₃-alkyl mono- or polysubstituted, identically or differently, with —NR¹²R¹³, preferably —N(C₁-C₃-alkyl)₂ or C₂-C₁₀-heterocycloalkyl, but preferably only C₁-C₃-alkyl, [sic: substituted with] C₂-C₁₀-heterocycloalkyl, wherein the heterocycloalkyl contains at least one atom in the ring, identically or differently, from the group nitrogen, oxygen or sulfur and in a preferred variation contains at least one nitrogen and can be optionally interrupted in the ring by one or several —(CO)— or —SO₂— groups and optionally one or several double bonds can be contained in the ring.
Another preferred object of the third embodiment of the present invention are compounds of the general formula I, as described in any of claims 11 or 12, in which R⁷ represents C₁-C₃-alkyl mono- or polysubstituted, identically or differently, with —N(C₁-C₃-alkyl)₂, pyrrolidinyl, morpholinyl or piperidinyl.
Another preferred object of the third embodiment of the present invention are compounds of the general formula I, as described in any of claims 11 or 12, in which R⁷ represents ethyl optionally mono- or polysubstituted, identically or differently, with —N(C₁-C₃-alkyl)₂, pyrrolidinyl, morpholinyl or piperidinyl.
Another preferred object of the third embodiment of the present invention are compounds of the general formula I, as described in any of claims 11 or 12, in which R⁷ represents ethyl optionally mono- or polysubstituted, identically or differently, with —N(CH₃)₂, pyrrolidinyl, morpholinyl or piperidinyl.

### FOURTH EMBODIMENT OF THE PRESENT INVENTION

The task of the present compound in the third embodiment is to furnish improved compounds, improved particularly in the inhibition of polo-like kinases as compared to prior art and/or to provide compounds that have better physicochemical properties as compared to compounds disclosed in prior art.

In a fourth embodiment of the present invention it was found in claim 13, as described in claim 1 and/or 2, that compounds of the general formula I, in which the following mean
R³ represents M,
M represents the group —NR¹²—(CO)—R¹⁶,
R¹⁶ represents methyl mono- or polysubstituted, identically or differently, with C₁-C₄-alkoxyl, C₂-C₁₀-heterocycloalkyl, heteroaryl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the methyl itself can be optionally mono- or polysubstituted, identically or differently, with C₁ to C₃-alkyl, wherein the heterocycloalkyl contains at least one atom in the ring, identically or differently, from the group nitrogen, oxygen or sulfur and can be optionally interrupted in the ring by one or several —(CO)—, —(C═S)— or —SO₂— groups in the ring and optionally one or several double bonds can be contained in the ring and the ring itself can be optionally mono- or polysubstituted, identically or differently, with cyanogen, halogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl or C₁-C₃-alkoxyl,
as well as their solvates, hydrates, diastereomers, enantiomers and salts, solve the task of the present invention especially well.

Especially well suited to solve the task of the present invention according to this fourth embodiment therefore are compounds of the general formula I for which R³ represents M, M represents the group —NR¹²—(CO)—R¹⁶ and R¹⁶ represents methyl, wherein the methyl in turn is substituted at least with C₂-C₁₀-heterocycloalkyl, heteroaryl or with the group —NR¹⁰R¹¹ and the heterocycloalkyl and the heteroaryl contain at least one nitrogen.

Another variation of the fourth embodiment of the present invention are compounds of the general formula I in claim 14, as described in claim 13, in which
R¹⁶ represents methyl mono- or polysubstituted, identically or differently, with C₂-C₁₀-heterocycloalkyl, heteroaryl, or with the group —NR¹⁰R¹¹, wherein the methyl itself can be optionally mono- or polysubstituted, identically or differently, with C₁ to C₃-alkyl, wherein the heterocycloalkyl contains at least one atom in the ring, identically or differently, from the group nitrogen, oxygen or sulfur and can be optionally interrupted in the ring by one or several —(CO)—, —(C═S)— or —SO₂— groups and optionally one or several double bonds can be contained and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl or C₁-C₃-alkoxyl,
as well as their solvates, hydrates, diastereomers, enantiomers and salts.
An object of the fourth embodiment of the present invention are compounds of the general formula I, as described in any of claims 13 or 14, in which R³ represents M.
Another object of the fourth embodiment of the present invention are compounds of the general formula I, as described in any of claims 13 or 14, in which M represents the group —NR¹²—(CO)—R¹⁶.
Another object of the fourth embodiment of the present invention are compounds of the general formula I, as described in one of claims 13 or 14, in which for R¹⁶ represents methyl mono- or polysubstituted, identically or differently, with C₁-C₄-alkoxyl, C₂-C₁₀-heterocycloalkyl, heteroaryl, preferably without heteroaryl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the methyl itself can be optionally mono- or polysubstituted, identically or differently, with C₁ to C₃-alkyl, wherein the heterocycloalkyl contains at least one atom in the ring, identically or differently, from the group nitrogen, oxygen or sulfur and in a preferred variation contains at least one nitrogen, and can be optionally interrupted by one or several —(CO)—, —(C═S)—, preferably without —(C═S)—, or with —SO₂— groups in the ring and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl, preferably without C₁-C₃-alkyl, or C₁-C₃-alkoxyl,
Another object of the fourth embodiment of the present invention are compounds of the general formula I, as described in one of claims 13 or 14, in which for R¹⁶ represents methyl mono- or polysubstituted, identically or differently, with C₂-C₁₀-heterocycloalkyl, heteroaryl, preferably without heteroaryl, or with the group —NR¹⁰R¹¹, wherein the heterocycloalkyl contains at least one atom in the ring, identically or differently, from the group nitrogen, oxygen or sulfur and in a preferred variation contains at least one nitrogen, and can be optionally interrupted in the ring by one or several —(CO)—, —(C═S)—, preferably without —(C═S)—, or with —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen, C₁-C₃-alkyl, preferably without C₁-C₃-alkyl, or C₁-C₃-alkoxy. In a preferred variation the ring of the heterocycloalkyl is not substituted.
Another preferred object of the fourth embodiment of the present invention are compounds of the general formula I, as described in one of claims 13 or 14, in which R¹⁶ represents methyl mono- or polysubstituted, identically or differently, with pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, imidazolyl or benzimidazolyl, preferably without imidazolyl or benzimidazolyl, or with the group —NR¹⁰R¹¹, wherein pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves can be optionally mono- or polysubstituted, identically or differently, with halogen, hydroxyl, phenyl, or with the group —(CO)—R⁵, —(CO)—O—R⁵, —(SO₂)—R¹⁴, —N(CH₃)₂ or substituted with methyl or ethyl optionally mono- or polysubstituted, identically or differently, with halogen, hydroxyl, methylthiol or phenyl, wherein the phenyl itself can be optionally mono- or polysubstituted, identically or differently, with halogen, C₁-C₃-alkyl, preferably without C₁-C₃-alkyl or with C₁-C₃-alkoxy. In a preferred variation the pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, or the octahydroisochinolinyl are not substituted.

### FIFTH EMBODIMENT OF THE PRESENT INVENTION

In a fifth embodiment of the present invention it was found that compounds of the general formula I,

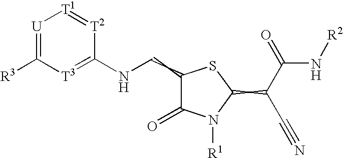

in which the following mean
T¹, T² and T³ independently represent —CH═ or —N═,
U represents —CR⁴═ or —N═,
R¹ represents C₁-C₃-alkyl or cyclopropyl optionally mono- or polysubstituted, identically or differently, with halogen,
R² represents C₁-C₃-alkyl, C₃-C₄-alkenyl, C₃-C₄-alkinyl or cyclopropyl optionally mono- or polysubstituted, identically or differently, with cyanogen, cyclopropyl, ethinyl or halogen or for hydroxyethyl at least monosubstituted with methyl,
R³ represents K, L or M,
K represents C₁-C₃-alkyl or C₂-C₄-alkenyl optionally mono- or polysubstituted, identically or differently, with X,
X represents halogen, hydroxyl or the group —OR⁶, —NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently, with halogen or C₁-C₃-alkoxyl,
L represents the group —O—R⁷, —O—(CH₂)ₙ—(CO)—NH—R⁸ or —O—(CH₂)ₙ—(CO)—O—R⁸,
M represents the group —NH—R⁹, —NH—(CO)—O—R⁹ or —NR¹²—(CO)—R⁹,
R⁴ represents hydrogen, cyanogen or halogen or represents methyl optionally mono- or polysubstituted, identically or differently, with halogen,
R⁵ represents C₁-C₄-alkyl, phenyl or —NR¹²R¹³,
R⁶ represents —SO₂—R¹⁴,
R⁷ represents C₁-C₃-alkyl optionally mono- or polysubstituted, identically or differently, with —NR¹²R¹³ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, aryl or with C₁-C₃-alkyl optionally mono- or polysubstituted, identically or differently, with halogen,
R⁸ represents C₁-C₃-alkyl, C₃-C₄-alkenyl or C₃-C₄-alkinyl optionally mono- or polysubstituted, identically or differently, with cyanogen, cyclopropyl or halogen,
R⁹ represents hydrogen or C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹²—(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently with halogen, or C₁-C₃-alkoxyl,
R¹⁰ and R¹¹ independently represent C₁-C₅-alkyl, C₂-C₁₀-heterocycloalkyl, aryl, —(CH₂)ₙ-aryl or heteroaryl optionally mono- or polysubstituted, identically or differently, with halogen, C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
R¹² and R¹³ independently represent hydrogen or C₁-C₄-alkyl,
R¹⁴ represents C₁-C₃-alkyl or aryl and
n is 1-4,
   as well as their solvates, hydrates, diastereomers, enantiomers and salts with the exception of:
2-[5-[1-(3-amino-phenylamino)-meth-(E/Z)-ylidene]-3-ethyl-4-oxo-thiazolidin-(2-(E or Z))-ylidene]-2-cyano-n-ethyl-acetamide,
2-cyano-n-ethyl-2-[3-ethyl-5-[1-{3-[2-(2-methoxy-ethoxy)-acetylamino]-phenylamino}-meth-(E/Z)-ylidene]-4-oxo-thiazolidin-(2-(E or Z))-ylidene]-acetamide,
2-cyano-2-[5-[1-[3-(2,2-dimethyl-propionylamino)-phenylamino]-meth-(E/Z)-ylidene]-3-ethyl-4-oxo-thiazolidin-(2-(E or Z))-ylidene]-n-ethyl-acetamide,
2-cyano-n-ethyl-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetamide,
2-cyano-2-[5-[1-[3-(2,2-dimethyl-propionylamino)-phenylamino]-meth-(E/Z)-ylidene]-3-ethyl-4-oxo-thiazolidin-(2-(E or Z))-ylidene]-n-(2-hydroxy-1,1-dimethyl-ethyl)-acetamide,
2-cyano-2-[5-[1-[3-(2,2-dimethyl-propionylamino)-phenylamino]-meth-(E/Z)-ylidene]-3-ethyl-4-oxo-thiazolidin-(2-(E or Z))-ylidene]-n-prop-2-ynyl-acetamide,
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-prop-2-ynyl-acetamide,
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2,2,2-trifluoro-ethyl)-acetamide,
2-cyano-n-cyclopropylmethyl-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetamide,
n-allyl-2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetamide,
2-cyano-2-[5-[1-[3-(2,2-dimethyl-propionylamino)-phenylamino]-meth-(E/Z)-ylidene]-3-ethyl-4-oxo-thiazolidin-(2-(E or Z))-ylidene]-n-methyl-acetamide
2-cyano-2-[5-[1-[3-(2,2-dimethyl-propionylamino)-phenylamino]-meth-(E/Z)-ylidene]-3-ethyl-4-oxo-thiazolidin-(2-(E or Z))-ylidene]-n-((s)-2-hydroxy-1-methyl-ethyl)-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2-methyl-allyl)-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2-methoxy-1-methyl-ethyl)-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2-hydroxy-propyl)-acetamide
2-cyano-n-cyclopropyl-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2-methoxy-ethyl)-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-propyl-acetamide
2-cyano-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-n-(2-hydroxy-1-methyl-ethyl)-acetamide
2-cyano-n-(cyano-dimethyl-methyl)-2-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetamide
   represent improved compounds, where the inhibition of polo-like kinases is concerned, that inhibit polo-like kinases in the nanomolecular range.

In particular those compounds of the general formula I are preferred in which
T¹, T² and T³ independently represent —CH═ or —N═,
U represents —CR⁴═ or —N═,
R¹ represents C₁-C₃-alkyl or cyclopropyl optionally mono- or polysubstituted, identically or differently, with halogen,
R² represents C₁-C₃-alkyl, C₃-C₄-alkenyl, C₃-C₄-alkinyl or cyclopropyl optionally mono- or polysubstituted, identically or differently, with cyanogen, cyclopropyl, ethinyl or halogen or for hydroxyethyl at least monosubstituted with methyl,
R³ represents K, L or M,
K represents C₁-C₃-alkyl or C₂-C₄-alkenyl optionally mono- or polysubstituted, identically or differently, with X,
X represents halogen, hydroxyl or the group —OR⁶, —NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxyl,
L represents the group —O—R⁷, —O—(CH₂)ₙ—(CO)—NH—R⁸ or —O—(CH₂)ₙ—(CO)—O—R⁸,
M represents the group —NH—R⁹, —NH—(CO)—O—R⁹ or —NR¹²—(CO)—R⁹,
R⁴ represents hydrogen, cyanogen or halogen or represents methyl optionally mono- or polysubstituted, identically or differently, with halogen,
R⁵ represents C₁-C₄-alkyl, phenyl or —NR¹²R¹³
R⁶ represents —SO₂—R¹⁴,
R⁷ represents C₁-C₃-alkyl optionally mono- or polysubstituted, identically or differently, with —NR¹²R¹³ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
R⁸ represents C₁-C₃-alkyl, C₃-C₄-alkenyl or C₃-C₄-alkinyl optionally mono- or polysubstituted, identically or differently, with cyanogen, cyclopropyl or halogen,
R⁹ represents hydrogen or C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, phenyl, which themselves can be optionally mono- or polysubstituted, identically or differently, with halogen or C₁-C₃-alkoxyl, or with the —(CO)—R⁵, —(CO)—O—R¹², —(SO₂)—R¹⁴, —NR¹²R¹³ or with C₁-C₃-alkyl optionally mono- or polysubstituted, identically or differently, with halogen, hydroxyl, C₁-C₃-alkylthiol or phenyl,
R¹⁰ and R¹¹ independently represent C₁-C₅-alkyl, C₂-C₁₀-heterocycloalkyl, aryl, or heteroaryl optionally mono- or polysubstituted, identically or differently, with halogen, C₁-C₃-alkyl, C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
R¹² and R¹³ independently represent hydrogen or C₁-C₄-alkyl,
R¹⁴ represents C₁-C₃-alkyl or aryl and
n is 1-4,
   as well as their solvates, hydrates, diastereomers, enantiomers and salts.

The following compounds of the general formula I are also preferred, in which
T¹, T² and T³ independently represent —CH═ or —N═,
U represents —CR⁴═ or —N═,
R¹ represents methyl, ethyl, isopropyl or cyclopropyl optionally mono- or polysubstituted, identically or differently, with halogen,
R² represents methyl, ethyl, allyl, propargyl optionally mono- or polysubstituted, identically or differently, with cyanogen, cyclopropyl, ethinyl or halogen or hydroxyethyl at least monosubstituted with methyl,
R³ represents K, L or M,
K represents C₁-C₃-alkyl or C₂-C₄-alkenyl optionally mono- or polysubstituted, identically or differently, with X,
X represents halogen, hydroxyl or the group —OR⁶, —NR¹⁰R¹¹ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves are mono- or polysubstituted, identically or differently with halogen, hydroxyl, phenyl, which themselves can be optionally mono- or polysubstituted, identically or differently, with halogen or C₁-C₃-alkoxyl, or with the group —(CO)—R⁵, —NR¹²R¹³ or with C₁-C₃-alkyl mono- or polysubstituted, identically or differently, with cyanogen, halogen or C₁-C₃-alkylthiol,
L represents the group —O—R⁷, —O—(CH₂)ₙ—(CO)—NH—R⁸ or —O—(CH₂)ₙ—(CO)—O—R⁸,
M represents the group —NH—R⁹, —NH—(CO)—R⁹, —NH—(CO)—O—R⁹ or —NR¹²—(CO)—R⁹,
R⁴ represents hydrogen or halogen or methyl optionally mono- or polysubstituted, identically or differently, with halogen,
R⁵ represents methyl, ethyl, tert.-butyl, phenyl or —NH₂,
R⁶ represents —SO₂-methyl,
R⁷ represents C₁-C₃-alkyl optionally mono- or polysubstituted, identically or differently, with —N(C₁-C₃-alkyl)₂, pyrrolidinyl, morpholinyl or piperidinyl,
R⁸ represents methyl, ethyl, allyl or propargyl optionally mono- or polysubstituted, identically or differently, with cyanogen, cyclopropyl or halogen,
R⁹ represents hydrogen or methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, cyanogen, cyclopropyl, halogen, hydroxyl or with the group —NR¹⁰R¹¹, —O—(CO)—R⁵, —(SO₂)—R¹⁴ or —O—(SO₂)—C₁-C₃-alkyl, wherein pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves can be optionally mono- or polysubstituted, identically or differently, with halogen, hydroxyl, phenyl or C₁-C₃-alkoxyl, or with the group —(CO)—R⁵, —(CO)—O—R⁵, —(SO₂)—R¹⁴, —N(CH₃)₂ or with methyl or ethyl optionally mono- or polysubstituted, identically or differently, with halogen, hydroxyl, methylthiol or phenyl,
R¹⁰ and R¹¹ independently represent C₁-C₅-alkyl, pyrrolidinyl, phenyl or pyridinyl optionally mono- or polysubstituted, identically or differently, with halogen, C₁-C₃-alkyl, or C₁-C₃-alkoxyl,
R¹² and R¹³ independently represent hydrogen or methyl, ethyl, isopropyl,
R¹⁴ represents C₁-C₄-alkyl or phenyl and
n is 1 or 2,
   as well as their solvates, hydrates, diastereomers, enantiomers and salts.

The following compounds of the general formula I are also preferred, in which
T¹, T² and T³ independently represent —CH═ or —N═,
U represents —CH═, —CF═, —C(CH₃)= or —N═,
R¹ represents methyl, ethyl, isopropyl or cyclopropyl optionally mono- or polysubstituted, identically or differently, with fluorine,
R² represents methyl, ethyl, allyl, propargyl optionally mono- or polysubstituted, identically or differently, with cyanogen, cyclopropyl, ethinyl or fluorine or hydroxyethyl at least monosubstituted with methyl,
R³ represents K, L or M,
K represents methyl, ethyl or ethenyl optionally mono- or polysubstituted, identically or differently, with X,
X represents halogen, hydroxyl or the group —O—SO₂-methyl or R⁶, —NR¹⁰R¹¹ or pyrrolidinyl, morpholinyl, thiomorpholinyl, piperidinyl or octahydroisochinolinyl, wherein pyrrolidinyl, morpholinyl, thiomorpholinyl, piperidinyl, octahydroisochinolinyl themselves can be optionally mono- or polysubstituted, identically or differently, with halogen, hydroxyl, phenyl, or with methyl optionally mono- or polysubstituted, identically or differently, with halogen,
L represents the group —O—R⁷, —O—(CH₂)—(CO)—NH—R⁸ or —O—(CH₂)—(CO)—O—R⁸,
M represents the group —NH₂—NH—R⁹, —NH—(CO)—R⁹, —NH—(CO)—O—R⁹ or —N(CH₃)—(CO)—R⁹,
R⁷ represents ethyl optionally mono- or polysubstituted, identically or differently, with —N(C₁-C₃-alkyl)₂, pyrrolidinyl, morpholinyl or piperidinyl,
R⁸ represents methyl, ethyl, allyl or propargyl optionally mono- or polysubstituted, identically or differently, with cyanogen, cyclopropyl or fluorine,
R⁹ represents hydrogen or methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, thiomorpholinyl, cyanogen, cyclopropyl, halogen, hydroxyl or with the group —N(C₁-C₃-alkyl)₂, —O—(CO)—(C₁-C₃-alkyl) or —O—(SO₂)—C₁-C₃-alkyl, wherein pyrrolidinyl, piperidinyl, piperazinyl, morpholinyl, thiomorpholinyl, themselves can be optionally mono- or polysubstituted, identically or differently, with halogen or with the group —(CO)—C₁-C₄-alkyl, —(CO)—O—C₁-C₄-alkyl, —(SO₂)—C₁-C₃-alkyl, —(SO₂)-phenyl, —N(C₁-C₃-alkyl)₂ or with methyl or ethyl optionally mono- or polysubstituted, identically or differently, with halogen, hydroxyl or C₁-C₃-alkylthiol,
   as well as their solvates, hydrates, diastereomers, enantiomers and salts.

Those compounds of the general formula I are in turn also preferred, in which the following mean
T¹, T² and T³ independently represent —CH═ or —N═,
U represents —CH═, —CF═, —C(CH₃)= or —N═,
R¹ represents ethyl,
R² represents methyl, ethyl, allyl, propargyl optionally mono- or polysubstituted, identically or differently, with cyanogen, cyclopropyl, ethinyl or fluorine or hydroxyethyl at least monosubstituted with methyl,
R³ represents K, L or M,
K represents methyl, ethyl or ethenyl optionally mono- or polysubstituted, identically or differently, with X,
X represents iodine, hydroxyl or the group —O—SO₂-methyl or pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently, with halogen, hydroxyl, phenyl, or with methyl optionally mono- or polysubstituted, identically or differently, with halogen,
L represents the group —O—R⁷, —O—(CH₂)—(CO)—NH—R⁸ or —O—(CH₂)—(CO)—O—R⁸
M represents the group —NH₂—NH—R⁹, —NH—(CO)—R⁹, —NH—(CO)—O—R⁹, —N(CH₃)—(CO)—R⁹ or —N(CH₃)—R⁹,
R⁷ represents ethyl optionally mono- or polysubstituted, identically or differently, with —N(CH₃)₂, pyrrolidinyl, morpholinyl or piperidinyl,
R⁸ represents methyl, ethyl, allyl or propargyl optionally mono- or polysubstituted, identically or differently, with cyanogen, cyclopropyl or fluorine,
R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with methoxy, ethoxy, butoxy-ethoxy, methoxy-ethoxy, pyrrolidinyl, piperidinyl, piperazinyl, thiomorpholinyl, cyanogen, cyclopropyl, halogen, hydroxyl or the group —N(CH₃)₂, —N(CH₃)(C₂H₅), —O—(CO)—(CH₃) or —O—(SO₂)-methyl, wherein pyrrolidinyl, piperidinyl, piperazinyl, morpholinyl, thiomorpholinyl, themselves can be optionally mono- or polysubstituted, identically or differently, with fluorine or with the group —(CO)—CH₃, —(CO)—C₂H₅, —(CO)—C(CH₃)₃, —(CO)—O—C(CH₃)₃, —(SO₂)—CH₃, —(SO₂)-phenyl, —N(CH₃)₂ or with methyl or ethyl optionally mono- or polysubstituted, identically or differently, with fluorine, hydroxyl or methylthiol,
   as well as their solvates, hydrates, diastereomers, enantiomers and salts.

Moreover, preferred among those are those compounds of the general formula I, in which the following mean
T¹, T² and T³ independently represent —CH═ or —N═ and T2 can also represent (—CF)═ and at least one substituent of T¹, T² or T³ represents —N═,
U represents —CH═,
R¹ represents ethyl,
R² represents methyl, ethyl or propargyl optionally mono- or polysubstituted, identically or differently, with cyanogen or fluorine or hydroxyethyl at least monosubstituted with methyl,
R³ represents M,
M represents the group —NH—(CO)—R⁹,
R⁹ represents methyl or tert.-butyl optionally substituted with methoxy-ethoxy,
   as well as their solvates, hydrates, diastereomers, enantiomers and salts.

Other preferred compounds of the general formula I are those in which
T¹, T² and T³ represent —CH═,
U represents —N═,
R¹ represents ethyl,
R² represents methyl, ethyl, propargyl optionally mono- or polysubstituted, identically or differently, with cyanogen or fluorine or for hydroxyethyl at least monosubstituted with methyl,
R³ represents M,
M represents the group —NH—R⁹,
R⁹ represents ethyl,
   as well as their solvates, hydrates, diastereomers, enantiomers or salts.

The compounds of the general formula II are likewise objects of the invention

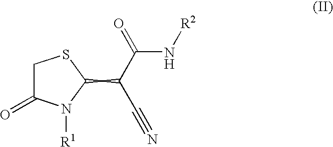

in which
R¹ and R² have the meanings set forth in general formula I, as well as their solvates, hydrates, diastereomers, enantiomers and salts as intermediate products.

### The Following Implementations Pertain to the First and the Second Embodiment of the Invention Similarly:

Another object of the first and the second embodiment of the present invention are compounds of the general formula I, as described in any of claims 1 through 10, in which R³ represents K, L or M or R¹⁵ and preferably in which R³ represents K, L or M.
Another object of the first and the second embodiment of the present invention are compounds of the general formula I, as described in any of claims 1 through 10, in which X represents halogen, hydroxyl or the group —OR⁶, —NR¹⁰R¹¹ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)—, —(C═S)— but preferably without —(C═S)—, or —SO₂— groups and optionally one or several double bonds can be contained in the ring and the ring itself can be optionally mono- or polysubstituted, identically or differently, with cyanogen, halogen, hydroxyl, aryl or with the group —(CO)—R⁵, —NR¹²R¹³ or with C₁-C₃-alkyl optionally mono- or polysubstituted, identically or differently, with halogen, hydroxyl or C₁-C₃-alkylthiol, wherein the aryl itself can be optionally mono- or polysubstituted, identically or differently, with cyanogen, halogen or C₁-C₃-alkoxy.
Another preferred object of the first and second embodiments of the present invention are compounds of the general formula I, as described in any of claims 1 through 10, in which X represents halogen, hydroxyl or the group —OR⁶—NR¹⁰R¹¹ or azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, wherein pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves can be optionally mono- or polysubstituted, identically or differently, with halogen, hydroxyl or phenyl, which themselves can be optionally mono- or polysubstituted, identically or differently with halogen or C₁-C₃-alkoxyl, or with the group —(CO)—R⁵, —NR¹²R¹³ or with C₁-C₃-alkoxy optionally mono- or polysubstituted, identically or differently, with cyanogen, halogen, hydroxyl, or C₁-C₃-alkylthiol.
Another preferred object of the first and second embodiments of the present invention are compounds of the general formula I, as described in any of claims 1 through 10, in which X represents halogen, hydroxyl or the group —O—SO₂-methyl or pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently with halogen, hydroxyl, phenyl or methyl optionally mono- or polysubstituted, identically or differently, with halogen.
Another preferred object of the first and second embodiments of the present invention are compounds of the general formula I, as described in any of claims 1 through 10, in which X represents iodine, hydroxyl or the group —O—SO₂-methyl or pyrrolidinyl, morpholinyl, thiomorpholinyl, piperidinyl or octahydroisochinolinyl, wherein pyrrolidinyl, morpholinyl, thiomorpholinyl, piperidinyl or octahydroisochinolinyl themselves can be optionally mono- or polysubstituted, identically or differently with halogen, hydroxyl, phenyl or with methyl optionally mono- or polysubstituted, identically or differently, with halogen.
Another object of the first and the second embodiment of the present invention are compounds of the general formula I, as described in any of claims 1 through 10, in which M represents the group —NH—R⁹, —NH—(CO)—OH, —NH—(CO)—O—R⁹ or —NR¹²—(CO)—R¹⁶.
Another preferred object of the first and the second embodiment of the present invention are compounds of the general formula I, as described in any of claims 1 through 10, in which M represents the group —NH—R⁹, —NH—(CO)—R¹⁶, —NH—(CO)—O—R⁹ or —N(CH₃)—(CO)—R¹⁶.
Another object of the first and second embodiments of the present invention are compounds of the general formula I, as described in any of claims 1 through 10, in which R⁷ represents C₁-C₃-alkoxy optionally mono- or polysubstituted, identically or differently, with —NR¹²R¹³ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, aryl or with the group —(CO)—R⁵, or with C₁-C₃-alkyl optionally mono- or polysubstituted, identically or differently, with halogen.
Another object of the first and second embodiments of the present invention are compounds of the general formula I, as described in any of claims 1 through 10, in which R⁷ represents C₁-C₃-alkyl optionally mono- or polysubstituted, identically or differently, with —NR¹²R¹³ or C₂-C₁₀-heterocycloalkyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring.
Another preferred object of the first and second embodiments of the present invention are compounds of the general formula I, as described in any of claims 1 through 10, in which R⁷ represents C₁-C₃-alkyl optionally mono- or polysubstituted, identically or differently, with —N(C₁-C₃-alkyl)₂, pyrrolidinyl, morpholinyl or piperidinyl.
Another preferred object of the first and second embodiments of the present invention are compounds of the general formula I, as described in any of claims 1 through 14, in which R⁷ represents ethyl optionally mono- or polysubstituted, identically or differently, with —N(C₁-C₃-alkyl)₂, pyrrolidinyl, morpholinyl or piperidinyl.
Another preferred object of the first and second embodiments of the present invention are compounds of the general formula I, as described in any of claims 1 through 14, in which R⁷ represents ethyl optionally mono- or polysubstituted, identically or differently, with —N(CH₃)₂, pyrrolidinyl, morpholinyl or piperidinyl.

### The Following Implementations Pertain to the Third and Fourth Embodiments of the Invention Similarly:

Another object of the third and fourth embodiments of the present invention are compounds of the general formula I, as described in any of claims 11 through 14, in which R⁵ represents C₁-C₄-alkyl, phenyl or —NR¹²R¹³.
Another preferred object of the third and fourth embodiments of the present invention are compounds of the general formula I, as described in any of claims 11 through 14, in which R⁵ stands or methyl, ethyl, tert.-butyl, phenyl or —NH₂.

### The Following Implementations Pertain to the First Four Embodiments of the Invention Similarly:

An object of the present invention as described in the first four embodiments are compounds of the general formula I, as described in any of claims 1 through 14, in which T¹, T² and T³ independently represent —CH═ or —N=and T² can also represent (—CF)═.
An object of the present invention as described in the first four embodiments are compounds of the general formula I, as described in any of claims 1 through 14, in which T¹, T² and T³ independently represent —CH═ or —N═.
Another object of the present invention as described in the first four embodiments are compounds of the general formula I, as described in any of claims 1 through 14, in which U represents —CR⁴═ or —N═.
Another preferred object of the present invention as described in the first four embodiments are compounds of the general formula I, as described in any of claims 1 through 14, in which U represents —CH═, —CF═, —C(CH₃)= or —N═.
Another object of the present invention as described in the first four embodiments are compounds of the general formula I, as described in any of claims 1 through 14, in which R¹ represents C₁-C₃-alkyl or cyclopropyl optionally mono- or polysubstituted, identically or differently, with halogen,
Another object of the present invention as described in the first four embodiments are compounds of the general formula I, as described in any of claims 1 through 14, in which R¹ represents methyl, ethyl, isopropyl or cyclopropyl optionally mono- or polysubstituted, identically or differently, with halogen.
Another preferred object of the present invention as described in the first four embodiments are compounds of the general formula I, as described in any of claims 1 through 14, in which R¹ represents methyl, ethyl, isopropyl, or cyclopropyl optionally mono- or polysubstituted, identically or differently, with fluorine.
Another preferred object of the present invention as described in the first four embodiments are compounds of the general formula I, as described in any of claims 1 through 14, in which R¹ represents ethyl.
Another object of the first four embodiments of the present invention are compounds of the general formula I, as described in any of claims 1 through 14, in which R² represents C₁-C₃-alkyl, C₃-C₄-alkenyl, C₃-C₄-alkinyl or cyclopropyl optionally mono- or polysubstituted, identically or differently, with cyanogen, cyclopropyl, ethinyl or halogen or for hydroxyethyl at least monosubstituted with methyl.
Another object of the first four embodiments of the present invention are compounds of the general formula I, as described in any of claims 1 through 14, in which R² represents methyl, ethyl, allyl, or propargyl optionally mono- or polysubstituted, identically or differently, with cyanogen, cyclopropyl, ethinyl or halogen or hydroxyethyl at least monosubstituted with methyl.
Another preferred object of the first four embodiments of the present invention are compounds of the general formula I, as described in any of claims 1 through 14, in which R² represents methyl, ethyl, allyl or propargyl optionally mono- or polysubstituted, identically or differently, with cyanogen, cyclopropyl, ethinyl or fluorine or hydroxyethyl at least monosubstituted with methyl.
An object of the invention as described in the first four embodiments are compounds of the general formula (I), as described in any of claims 1 through 14, in which N represents 1 through 4. Another object of the present invention as per the first four embodiments are compounds of the general formula (I), as described in any of claims 1 through 14, in which N means 1 through 3. Another object of the present invention as per the first four embodiments are compounds of the general formula (I), as described in any of claims 1 through 14, in which N represents 1 through 2. Another object of the present invention as per the first four embodiments are compounds of the general formula (I), as described in any of claims 1 through 14, in which N represents 1.
Another object of the first four embodiments of the present invention are compounds of the general formula I, as described in any of claims 1 through 14, in which R⁴ represents hydrogen, cyanogen or halogen or methyl optionally mono- or polysubstituted, identically or differently, with halogen.
Another preferred object of the first four embodiments of the present invention are compounds of the general formula I, as described in any of claims 1 through 14, in which R⁴ represents hydrogen or halogen, or methyl optionally mono- or polysubstituted, identically or differently, with halogen.
Another object of the first four embodiments of the present invention are compounds of the general formula I, as described in any of claims 1 through 14, in which R⁶ represents —SO₂—R¹⁴.
Another preferred object of the first four embodiments of the present invention are compounds of the general formula I, as described in any of claims 1 through 14, in which R⁶ represents —SO₂-methyl.
Another object of the first four embodiments of the present invention are compounds of the general formula I, as described in any of claims 1 through 14, in which R⁸ stands C₁-C₃-alkyl, C₃-C₄-alkenyl or C₃-C₄-alkinyl optionally mono- or polysubstituted, identically or differently, with cyanogen, cyclopropyl or halogen.
Another object of the first four embodiments of the present invention are compounds of the general formula I, as described in any of claims 1 through 14, in which R⁸ represents methyl, ethyl, allyl or propargyl optionally mono- or polysubstituted, identically or differently, with cyanogen, cyclopropyl or halogen.
Another preferred object of the first four embodiments of the present invention are compounds of the general formula I, as described in any of claims 1 through 14, in which R⁸ represents methyl, ethyl, allyl or propargyl optionally mono- or polysubstituted, identically or differently, with cyanogen, cyclopropyl or fluorine.
Another object of the first four embodiments of the present invention are compounds of the general formula I, as described in any of claims 1 through 14, in which R⁹ represents C₁-C₅-alkyl, preferably C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹⁴, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —SO₂—R¹⁴, —NR¹²R¹³ or with C₁-C₃-alkyl optionally mono- or polysubstituted, identically or differently, with halogen, hydroxyl, C₁-C₃-alkylthiol or phenyl, wherein the aryl itself can be optionally mono- or polysubstituted, identically or differently, with halogen or C₁-C₃-alkoxy.
Another object of the first four embodiments of the present invention are compounds of the general formula I, as described in any of claims 1 through 14, in which R⁹ represents C₁-C₅-alkyl, preferably C₁-C₄-alkyl, C₂-C₄-alkenyl, cyclopropyl or C₂-C₁₀-heterocycloalkyl optionally mono- or polysubstituted, identically or differently, with C₁-C₄-alkoxyl, C₁-C₄-alkoxy-C₁-C₄-alkoxyl, C₂-C₁₀-heterocycloalkyl, cyanogen, cyclopropyl, halogen, hydroxyl or with the group —NR¹⁰R¹¹, —O—(CO)—R⁵, —(SO₂)—R¹⁴ or —O—(SO₂)—R¹², wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring and the ring itself can be optionally mono- or polysubstituted, identically or differently, with halogen, cyanogen, hydroxyl, aryl or with the group —(CO)—R⁵, —(CO)—O—R¹², —SO₂—R¹⁴, —NR¹²R¹³ or with C₁-C₃-alkyl optionally mono- or polysubstituted, identically or differently, with halogen, hydroxyl, C₁-C₃-alkylthiol or phenyl.
Another preferred object of the first four embodiments of the present invention are compounds of the general formula I, as described in any of claims 1 through 14, in which R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, cyanogen, cyclopropyl, halogen, hydroxyl or with the group —NR¹⁰R¹¹, —O—(CO)—R⁵, —(SO₂)—R¹⁴ or —O—(SO₂)—C₁-C₃-alkyl, wherein pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves can be optionally mono- or polysubstituted, identically or differently, with halogen, hydroxyl, phenyl or C₁-C₃-alkoxyl, or with the group —(CO)—R⁵, —(CO)—O—R⁵, —(SO₂)—R¹⁴, —N(CH₃)₂ or with methyl or ethyl optionally mono- or polysubstituted, identically or differently, with halogen, hydroxyl, methylthiol or phenyl.
Another preferred object of the first four embodiments of the present invention are compounds of the general formula I, as described in any of claims through 14, in which R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with C₁-C₄-alkoxyl, C₁-C₄-alkoxy-C₁-C₄-alkoxyl, pyrrolidinyl, piperidinyl, piperazinyl, morpholinyl, thiomorpholinyl, cyanogen, cyclopropyl, halogen, hydroxyl or with the group —N(C₁-C₃-alkyl)₂, —O—(CO)—(C₁-C₃-alkyl) or —O—(SO₂)—C₁-C₃-alkyl, wherein pyrrolidinyl, piperidinyl, piperazinyl, morpholinyl, thiomorpholinyl themselves can be optionally mono- or polysubstituted, identically or differently, with halogen or with the group —(CO)—C₁-C₄-alkyl, —(CO)—O—C₁-C₄-alkyl, —(SO₂)—C₁-C₃-alkyl, —(SO₂)-phenyl, —N(C₁-C₃-alkyl)₂ or with methyl or ethyl optionally mono- or polysubstituted, identically or differently, with halogen, hydroxyl or C₁-C₃-alkylthiol.
Another preferred object of the first four embodiments of the present invention are compounds of the general formula I, as described in any of claims 1 through 14, in which R⁹ represents methyl, ethyl, isopropyl, isobutyl, tert.-butyl, ethenyl, cyclopropyl, tetrahydropyranyl or tetrahydrofuranyl optionally mono- or polysubstituted, identically or differently, with methoxy, ethoxy, butoxy-ethoxy, methoxy-ethoxy, pyrrolidinyl, piperidinyl, piperazinyl, morpholinyl, thiomorpholinyl, cyanogen, cyclopropyl, chlorine, fluorine, hydroxyl or with the group —N(CH₃)₂, —N(CH₃)(C₂H₅), —O—(CO)—(CH₃) or —O—(SO₂)-methyl, wherein pyrrolidinyl, piperidinyl, piperazinyl, morpholinyl, thiomorpholinyl themselves can be optionally mono- or polysubstituted, identically or differently, with fluorine, or with the group —(CO)—CH₃, —(CO)—C₂H₅, —(CO)—C(CH₃)₃, —(CO)—O—C(CH₃)₃, —(SO₂)—CH₃, —(SO₂)-phenyl, —N(CH₃)₂ or with methyl or ethyl optionally mono- or polysubstituted, identically or differently, with fluorine, hydroxyl or methylthiol.
Another object of the first four embodiments of the present invention are compounds of the general formula I, as described in any of claims 1 through 14, in which R¹⁰ and R¹¹ independently represent C₁-C₅-alkyl, C₂-C₁₀-heterocycloalkyl, aryl, —(CH₂)ₙ-aryl or heteroaryl optionally mono- or polysubstituted, identically or differently, with halogen, C₁-C₃-alkyl or C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring,
Another object of the first four embodiments of the present invention are compounds of the general formula I, as described in any of claims 1 through 14, in which R¹⁰ and R¹¹ independently represent C₁-C₅-alkyl, C₂-C₁₀-heterocycloalkyl, aryl or heteroaryl optionally mono- or polysubstituted, identically or differently, with halogen, C₁-C₃-alkyl or C₁-C₃-alkoxyl, wherein the heterocycloalkyl contains at least one atom in the ring, identically or differently, from the group nitrogen, oxygen or sulfur and can be optionally interrupted in the ring by one or several —(CO)— or —SO₂— groups and optionally one or several double bonds can be contained in the ring.
Another preferred object of the first four embodiments of the present invention are compounds of the general formula I, as described in any of claims 1 through 14, in which R¹⁰ and R¹¹ independently represent C₁-C₅-alkyl, pyrrolidinyl, phenyl or pyridinyl optionally mono- or polysubstituted, identically or differently with halogen, C₁-C₃-alkyl or C₁-C₃-alkoxy.
Another object of the first four embodiments of the present invention are compounds of the general formula I, as described in any of claims 1 through 14, in which R¹² and R¹³ independently represent hydrogen or C₁-C₄-alkyl.
Another preferred object of the first four embodiments of the present invention are compounds of the general formula I, as described in any of claims 1 through 14, in which R¹² and R¹³ independently represent hydrogen or methyl, ethyl, or isopropyl.
Another object of the first four embodiments of the present invention are compounds of the general formula I, as described in any of claims 1 through 14, in which R¹⁴ represents C₁-C₃-alkyl or aryl.
Another object of the first four embodiments of the present invention are compounds of the general formula I, as described in any of claims 1 through 14, in which R¹⁴ represents C₁-C₃-alkyl or phenyl.
Another preferred object of the first four embodiments of the present invention are compounds of the general formula I, as described in any of claims 1 through 14, in which R¹⁴ represents C₁-C₄-alkyl or phenyl.
Another object of the first four embodiments of the present invention are compounds of the general formula I, as described in any of claims 1 through 14, in which R¹⁵ represents a C₂-C₁₀-heterocycloalkyl optionally mono- or polysubstituted, identically or differently, with C₁-C₃-alkyl or —(CH₂)ₙ-aryl, wherein the heterocycloalkyl contains at least one atom in the ring, identically or differently, from the following group, oxygen or sulfur, and can be optionally interrupted in the ring by one or several —(CO)— or —SO₂— groups and optionally one or several double bonds can be contained in the ring.
Another preferred object of the first four embodiments of the present invention are compounds of the general formula I, as described in any of claims 1 through 14, in which R¹⁵ represents a C₂-C₁₀-heterocycloalkyl optionally mono- or polysubstituted, identically or differently, with C₁-C₃-alkyl or —(CH₂)ₙ-phenyl, wherein the heterocycloalkyl contains at least one atom in the ring, identically or differently, from the following group, oxygen or sulfur.
Another object of the first four embodiments of the present invention are compounds of the general formula II or IV in claim 15,

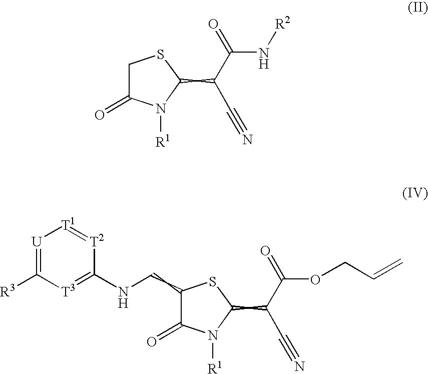

in which
R¹, R², R³, U, T¹, T² and R³ have the meaning shown in the general formula I, as described in any of claims 1 through 14, as well as their solvates, hydrates, diastereomers, enantiomers and salts as intermediate products for producing compounds of the general formula (I).

Another object of the first four embodiments of the present invention are compounds of the general formula II as per claim 15 in claim 16 with the following formulas:
2-cyanogen-n-ethyl-2-[3-ethyl-4-oxo-thiazolidin-(2-(E or Z))-ylidene]-acetamide,
2-cyanogen-2-[3-ethyl-4-oxo-thiazolidin-(2-(E or Z))-ylidene]-n-(2,2,2-trifluoro-ethyl)-acetamide,
2-cyanogen-2-[3-ethyl-4-oxo-thiazolidin-(2-(E or Z))-ylidene]-n-prop-2-ynyl-acetamide or
2-cyanogen-n-cyanogenmethyl-2-[3-ethyl-4-oxo-thiazolidin-(2-(E or Z))-ylidene]-acetamide
2-cyanogen-n-(2,2-difluoro-ethyl)-2-[3-ethyl-4-oxo-thiazolidin-(2-(E or Z))-ylidene]-acetamide
2-cyanogen-2-[3-ethyl-4-oxo-thiazolidin-(2-(E or Z))-ylidene]-n-(2-hydroxy-1,1-dimethyl-ethyl)-acetamide
2-cyanogen-2-[3-ethyl-4-oxo-thiazolidin-(2-(E or Z))-ylidene]-n-(2-fluoro-ethyl)-acetamide
   as well as their solvates, hydrates, diastereomers, enantiomers and salts as intermediate products for producing compounds of the general formula (I).

Another object of the first four embodiments of the present invention are compounds in claim 17 of the general formulas (II) or (IV) as described in claim 15 or compounds as described in claim 16 for use as intermediate products for producing compounds of the general formula (I).

Another object of the first four embodiments of the present invention are the use of the compounds of the general formulas (II) or (IV) in claim 18 as described in claim 15 or compounds as described in claim 16 as intermediate products for producing compounds of the general formula (I).

Another object of the first four embodiments of the present invention is drugs in claim 19 that contain at least one compound described in any of claims 1 through 14.

Another object of the first four embodiments of the present invention is the use of compounds of the general formula I in claim 20, as described in any of claims 1 through 14, for producing a drug.

Another object of the first four embodiments of the present invention are compounds in claim 21 described in any of claims 1 through 14 or the drug described in claim 19 with suitable formulation substances and carrier substances.

Another object of the first four embodiments of the present invention is a method in claim 22 for producing compounds of the general formula I, wherein compounds of the general formula II are heated with compounds of the general formula III,

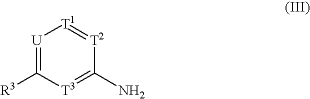

in which
R³, U, T¹, T² and T³ have the same meaning as R³, U, T¹, T² and T³ described in any of claims 1 through 14, in a formic acid orthoester with three identical or different alkoxy- or aryloxy residues optionally bridged or substituted with halogen and optionally a polar solvent, or
compounds of the general formula IV

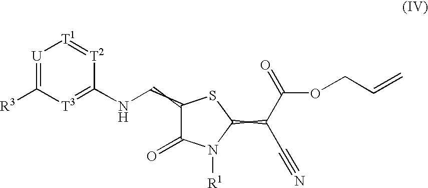

in which
R¹, R³, U, T¹, T² and T³ have the same meaning as R¹, R³, U, T¹, T² and T³ as described in any of claims 1 through 14, are converted with an allyl acceptor and a catalyst in an aprotic solvent and, after completion of a first partial reaction with a coupling reagent, a base and R²—NH₂, wherein R² has the same meaning as R² as set forth in any of claims 1 through 14, converted in an aprotic solvent into compounds of the general formula I.

Another object of the first four embodiments of the present invention is a method in claim 23, according to claim 22, wherein for producing the compounds of the general formula II, compounds of the general formula V,

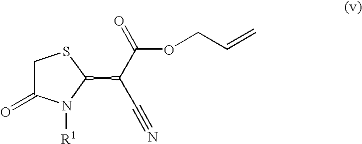

in which
R¹ has the same meaning as R¹ as described in any of claims 1 through 14, are converted with an allyl acceptor and a catalyst in an aprotic solvent and, after completion of a first partial reaction, converted with a coupling reagent, a base and R²—NH₂, wherein R² has the same meaning as R² as described in any of claims 1 through 14, and with an aprotic solvent into the compounds of the general formula I.

Understood under formic acid orthoester with three identical or different alkoxy residues optionally bridged or substituted with halogen, as described in either of claims 22 or 23, is preferably a triethylorthoformate. Other formic acid orthoesters that fall under this definition are known to people skilled in the field.

Polar solvents suitable for performing the method described in claim 22 are C₁ through C₅ alcohols or diols like e.g. glycol, preferably C₁ through C₅ alcohols and especially preferably ethanol or 1-propanol. If there is an excess of formic acid orthoester on hand, no polar solvent is needed to perform the reaction of the compounds of the general formula II with compounds of the general formula III to the compounds of the general formula I.

For reacting the compounds of the general formula II with compounds of the general formula III to the compounds with the general formula I as described in claim 22, they must be heated up. In a preferred variation, the reaction is supposed to occur at, at least, 70° C., more preferably between 70° C. and 150° C. and even more preferably between 100° C. and 150° C. The reaction can also be performed at higher temperatures, but then—as anyone skilled in the field knows—a higher-boiling solvent or pressure vessel should be used. In a preferred variation of the invention the heating reaction is performed for 2 to 24 hours.

“Catalysts” employable for the methods described in any of claims 22 or 23 are known to people skilled in the field. The use of a palladium catalyst is preferable.

“Aprotic solvents” employable for performing the methods of claims 22 or 23 are known to people skilled in the field. Tetrahydrofurane and dichloromethane are suitable aprotic solvents that are preferably used. In the coupling reaction (2^{nd} partial reaction) of claims 22 or 23, dimethylformamide can preferably also be used as an aprotic solvent. People skilled in the field also know, however, that other aprotic solvents like e.g. dimethylacetamide (DMA) and n-methylpyrrolidone (NMP) can also be used to perform the methods of claims 22 or 23.

Understood to be preferable allyl acceptors according to the present invention and according to claims 22 or 23 are 1,3-dimethylbarbituric acid, barbituric acid and/or a silane. People skilled in the field also are also aware of other allyl acceptors that can be used to perform the production method described.

“Coupling reagents” employable for performing the methods of claim 22 or 23 are known to people skilled in the field. Preferably used coupling reagents are 0-(BENZOTRIAZOL-1-YL)-N,N,N′,N′-TETRAMETHYLURONIUM TETRAFLUOROBORATE (TBTU) and/or O-(7-AZABENZOTRIAZOL-1-YL)-N,N,N′,N′-TETRAMETHYLURONIUM HEXAFLUORO-PHOSPHATES (HATU).

“Bases” employable for performing the methods of claims 22 or 23 are known to people skilled in the field. Preferably used bases are triethylamine, Hunig's base or sodiumhydrogencarbonate.

The reactions of compounds of the general formula IV to the compounds of the general formula I described in claim 22 and of compounds of the general formula V to compounds of the general formula II as described in claim 23, are preferably performed at a temperature of 0° C. to 50° C. and even more preferably at ambient temperature.

### The Following Implementations Pertain Similarly to all Embodiments of the Invention

Understood under alkyl is any straight-chained or branched alkyl residue, like e.g. methyl, ethyl, propyl, isopropyl, butyl, isobutyl, sec. butyl, tert. butyl, pentyl, isopentyl, hexyl, heptyl, octyl, nonyl and decyl.

Understood under alkoxyl is any straight-chained or branched alkoxyl residue, like e.g. methyloxy, ethyloxy, propyloxy, isopropyloxy, butyloxy, isobutyloxy, sec. butyloxy, pentyloxy, isopentyloxy, hexyloxy, heptyloxy, octyloxy, nonyloxy or decyloxy. Preferred in the present invention, however, are C₁-C₆-alkoxy groups, especially preferred are C₁-C₃-alkoxyl groups and especially preferred is a methoxyl group.

The alkenyl substituents are respectively straight-chained or branched, wherein e.g. the following residues are intended: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, allyl.

Understood under alkinyl is any straight-chained or branched alkinyl residue that contains 2-6, preferably 2-4 C-atoms. The following residues are given as examples: acetylene, propin-1-yl, propin-3-yl (propargyl), but-1-in-1-yl, but-1-in-4-yl, but-2-in-1-yl, but-1-in-3-yl, etc.

C₂-C₁₀-heterocycloalkyl represents an alkyl ring comprising 2-10 carbon atoms, preferably 3 to 10 carbon atoms and especially preferably 5 to 6 carbon atoms, wherein the heterocycloalkyl contains at least one atom in the ring, identically or differently, from the group oxygen, sulfur or nitrogen and can be optionally interrupted in the ring by one or several —(CO)—, —(CS)— or —SO₂— groups and optionally one or several double bonds can be contained in the ring and the ring itself can be optionally mono- or polysubstituted or annealed identically or differently.

Examples mentioned of heterocycloalkyls are: oxiranyl, oxethanyl, dioxolanyl, dithianyl, dioxanyl, aziridinyl, azetidinyl, tetrahydrofuranyl, tetrahydropyranyl, tetrahydrooxazolyl, tetrahydrothiazolyl, tetrahydroisochinolinyl, octahydroisochinolinyl, tetrahydrochinolinyl, octahydrochinolinyl, tetrahydroimidazolonyl, pyrazolidinyl, pyrrolidinyl, pyrrolidonyl, piperidinyl, piperazinyl, piperazinonyl, n-methylpyrrolidinyl, 2-hydroxymethylpyrrolidinyl, 3-hydroxypyrrolidinyl, n-methylpiperazinyl, n-acetylpiperazinyl, n-methylsulfonylpiperazinyl, 4-hydroxypiperidinyl, 4-aminocarbonylpiperidinyl, 2-hydroxyethylpiperidinyl, 4-hydroxymethylpiperidinyl, imidazolidinyl, tetrahydroimidazolonyl, morpholinyl, thiomorpholinyl, 1,1-dioxo-thiomorpholinyl, trithianyl, tetrahydrotriazinthionyl, triazinthionyl, chinuclidinyl, nortropinyl, etc.

or rings of the aforementioned, which are benzocondensed, like e.g. benzopyrrolidinyl, benzomorpholinyl, etc.

Substituents on the heterocycloalkylring can be e.g.:

cyanogen, halogen, hydroxyl, C₁-C₆-alkyl, C₁-C₆-alkoxyl, C₁-C₆-alkoxyalkyl, C₁-C₆-hydroxyalkyl, C₃-C₆-cycloalkyl, aryl or C₁-C₆-alkyl optionally mono- or polysubstituted, identically or differently with halogen, hydroxyl or C₁-C₆-alkylthiol, or with the group —(CO)—C₁-C₆-alkyl, —(CO)—O—C₁-C₆— alkyl, —(SO₂)—C₁-C₆— alkyl, —(SO₂)-phenyl, —NH₂, —N(C₁-C₆— alkyl)₂, —NH(C₁-C₆— alkyl) etc.

Understood under cycloalkyl are monocyclic alkyl rings like cyclopropyl, cyclobutyl, cyclopentyl, cyclohexyl or cycloheptyl, but also bicyclic rings or tricyclic rings like e.g. adamantanyl. The cycloalkyl may also be optionally benzocondensed, like e.g. (tetralin)yl, etc.

Understood under halogen are fluorine, chlorine, bromine or iodine respectively.

The heteroaryl residue comprises 5-16 ring atoms, preferably 5 to 10 ring atoms and especially preferably 5 to 7 ring atoms, and, instead of carbon, contain one or several, identical or different, heteroatoms, like oxygen, nitrogen or sulfur in the ring, and can be mono-, bi- or tricyclic, and can also be benzocondensed.

Examples mentioned are:

Thienyl, furanyl, pyrrolidinylyl, oxazolyl, thiazolyl, imidazolyl, pyrazolyl, isoxazolyl, isothiazolyl, oxadiazolyl, triazolyl, thiadiazolyl, etc. and benzoderivates thereof, like e.g. benzofuranyl, benzothienyl, benzoxazolyl, benzimidazolyl, indazolyl, indolyl, isoindolyl, etc.; or pyridyl, pyridazinyl, pyrimidinyl, pyrazinyl, triazinyl, etc. and benzoderivates thereof, like e.g. chinolyl, isochinolyl, etc.; or oxepinyl,
azocinyl, indolizinyl, indolyl, indolinyl, isoindolyl, indazolyl, benzimidazolyl, purinyl, etc. and benzoderivates thereof; or chinolinyl, isochinolinyl, cinnolinyl, phthalazinyl, chinazolinyl, chinoxalinyl, naphthyridinyl, pteridinyl, carbazolyl, acridinyl, phenazinyl, phenothiazinyl, phenoxazinyl, xanthenyl, tetralinyl etc.

Especially preferred heteroaryl residues are e.g. 5-ring heteroaromates like thiophene, furanyl, oxazolyl, thiazol, imidazolyl and benzoderivates thereof (like e.g. benzimidazolyl) and 6-ring heteroaromates like pyridinyl, pyrimidinyl, triazinyl, chinolinyl, isochinolinyl and benzoderivates thereof.

The aryl residue comprises respectively 3-12 carbon atoms and may be respectively substituted or benzocondensed.

Mentioned as examples: cyclopropenyl, cyclopentadienyl, phenyl, tropyl, cyclooctadienyl, indenyl, naphthyl, azulenyl, biphenyl, fluorenyl, anthracenyl, tetralinyl, tolyl etc.

As it is used in this application, “C₁-C₅” means, e.g. in connection with the definition of “C₁-C₅-alkyl”, an alkyl group with an end number of 1 to 5 carbon atoms, i.e. 1, 2, 3, 4 or 5 carbon atoms. The definition of “C₁-C₅” is further interpreted to include any possible subgroup, like e.g., C₁-C₅, C₂-C₅, C₃-C₅, C₄-C₅, C₁-C₂, C₁-C₃, C₁-C₄, C₁-C₅.

The information of the application regarding the different groups not explicitly listed here is defined in the same way as the “C₁-C₅” groups mentioned as examples above.

Understood under isomers are chemical compounds of the same sum formula but of a different chemical structure. A differentiation is generally made between isomers and stereoisomers.

Constitutional isomers possess the same sum formula, but are set apart, however, by how their atoms or atom groups link. These include functional isomers, position isomers, tautomers or valence isomers.

Stereoisomers have basically the same structure (constitution)—and therefore the same formula as well—but differ through the spatial configuration of the atoms. A differentiation is generally made between configurational isomers and conformational isomers. Configurational isomers are stereoisomers that can only be converted into each other by bond breakage. They include enantiomers, diastereomers and E/Z (cis/trans) isomers.

Enantiomers are stereoisomers that behave like an image to a mirror image and do not exhibit any plane of symmetry. All stereoisomers that are not enantiomers are called diastereomers. E/Z (cis/trans) isomers at double bonds are the special case. Conformational isomers are stereoisomers that can be converted into each other through single bond rotation.

For delineating isomery types from each other, see also the IUPAC rules, section E (Pure Appl. Chem. 45, 11-30, 1976).

The inventive compounds of the general formula I also include the possible tautomeric forms and include the E or Z isomers or, if there is a chiral center, the racemates and enantiomers as well. These are understood to include double bond isomers as well.

The compounds of the invention may also be in the form of solvates, particularly hydrates, wherein the compounds of the invention accordingly contain polar solvents, particularly of water, as a structural element of the crystal lattice of the compounds of the invention. The portion of polar solvent, in particular water, can be in stoichiometric or unstoichiometric ratio. For stoichiometric solvates or hydrates, hemi-, (semi-), mono-, sesqui-, di-, tri-, tetra-, penta-, etc. solvates or hydrates are also mentioned.

If there is an acidic function, physiologically compatible salts of organic and inorganic bases are suitable as salts, like e.g. well-soluble alkali- and earth alkali salts as well as n-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak-base, 1-amino-2,3,4-butantriol.

If it contains a basic function, the physiologically compatible salts of organic and inorganic acids are suitable, like hydrochloric acid, sulfuric acid, phosphoric acid, citric acid, tartaric acid, maleinic acid, fumaric acid, etc.

The compounds of the invention of the general formula I essentially inhibit polo-like kinases, on which their effect is also based, e.g. against cancer, like solid tumors and leukemia, autoimmune diseases like psoriasis, alopecia, and multiple sclerosis, chemotherapeutically-induced alopecia and mucositis, cardiovascular diseases like stenoses, arterioscleroses and restenoses, infectious diseases, like those brought upon e.g. by unicellular parasites like trypanosoma, toxoplasma or plasmodium, or by fungi, nephrological diseases like e.g. glomerulonephritis, chronic neurodegenerative diseases like Huntington's disease, amyotrophic lateral sclerosis, Parkinson's disease, AIDS-induced dementia and Alzheimer's disease, acute neurodegenerative diseases like cerebral ischemias and neurotraumas, viral infections like e.g. cytomegalus-infections, herpes, hepatitis B and C, and HIV diseases.

An object of the present invention is also the use of the compounds of the general formula II as well as their solvates, hydrates, diastereomers, enantiomers and salts as intermediate products.

To use the inventive compounds of the general formula I as a drug, they are brought into the form of a pharmaceutical preparation that, in addition to the agent for the enteral or parenteral application, contains pharmaceutical, organic or inorganic inert carrier materials, like e.g. water, gelatins, Arabian rubber, lactose, starch, magnesium stearate, talc, vegetable oils, polyalkylene glycols, etc. The pharmaceutical preparations may be in solid form, e.g. as tablets, dragees, suppositories, capsules or in liquid form, e.g. as solutions, suspensions or emulsions. In addition to that they also contain adjuvants, like preservatives, stabilizers or emulsifiers; salts to change osmotic pressure or buffers.

These pharmaceutical preparations are also an object of the present invention.

Particularly suited for parenteral application are injection solutions or suspensions, especially aqueous solutions of the active compounds in polyhydroxyethoxylated ricinus oil.

Surface-active adjuvants like salts of gallic acids or animal or vegetable phospholipids, but also mixtures thereof, as well as liposomes or their components can also be used as carrier systems.

Suited particularly for oral application are tablets, dragees or capsules with talcum and/or carbon hydrogen carriers or binders, like e.g. lactose, corn or potato starch. Application can also be done in liquid form, like e.g. as a juice with an optionally added sweetener.

The enteral, parenteral and oral applications are also an object of the present invention. The dosage of these agents can vary depending on the administration path, age and weight of the patient, type and severity of the disease being treated and similar factors. The daily dose is 0.5-1000 mg, preferably 50-200 mg, wherein the dose can be given as a one-time dose or divided into 2 or more daily doses.

Likewise an object of the present invention is the use of the compounds of the general formula I for producing a drug for treating cancer, autoimmune diseases, cardiovascular diseases, chemotherapeutically-induced alopecia and mucositis, infectious diseases, nephrological diseases, chronic and acute neurodegenerative diseases and viral infections, wherein understood under cancer are solid tumors and leukemia, understood under autoimmune diseases are psoriasis, alopecia and multiple sclerosis, understood under cardiovascular diseases are stenoses, arterial scleroses and restenoses, understood under infectious diseases are diseases brought about by unicellular parasites, understood under nephrological diseases are glomerulonephritis, understood under chronic neurodegenerative diseases are Huntington's disease, amyotrophic lateral sclerosis, Parkinson's disease, AIDS-induced dementia and Alzheimer's disease, understood under acute neurodegenerative diseases are cerebral ischemias and neurotraumas, and understood under viral infections are cytomegalus-infections, herpes, hepatitis B or C, and HIV diseases.

Likewise an object of the present invention are drugs for treating the diseases listed above that contain at least one compound of the general formula I as well as drugs with suitable formulation and carrier substances.

The compounds of the invention of general formula I are among other things excellent inhibitors of polo-like kinases, like PLK 1, PLK 2, PLK 3 and PLK 4.

Where the production of the starting compounds is not described, they are known or similar to known compounds or producible according to methods described here. It is also possible to perform all the conversions described here in parallel reactors or by means of combined work methods.

The isomer mixtures can be separated according to standard methods like e.g. crystallization, chromatography or salification into isomers, like e.g. into enantiomers, diastereomers or E/Z isomers as long as the isomers do not stand in equilibrium with each other.

The salts are produced in the standard way by mixing a solution of the compound of formula I with the equivalent amount or an excess of a base or acid that is preferably in solution and separating off the precipitate or preparing the solution in the standard manner.

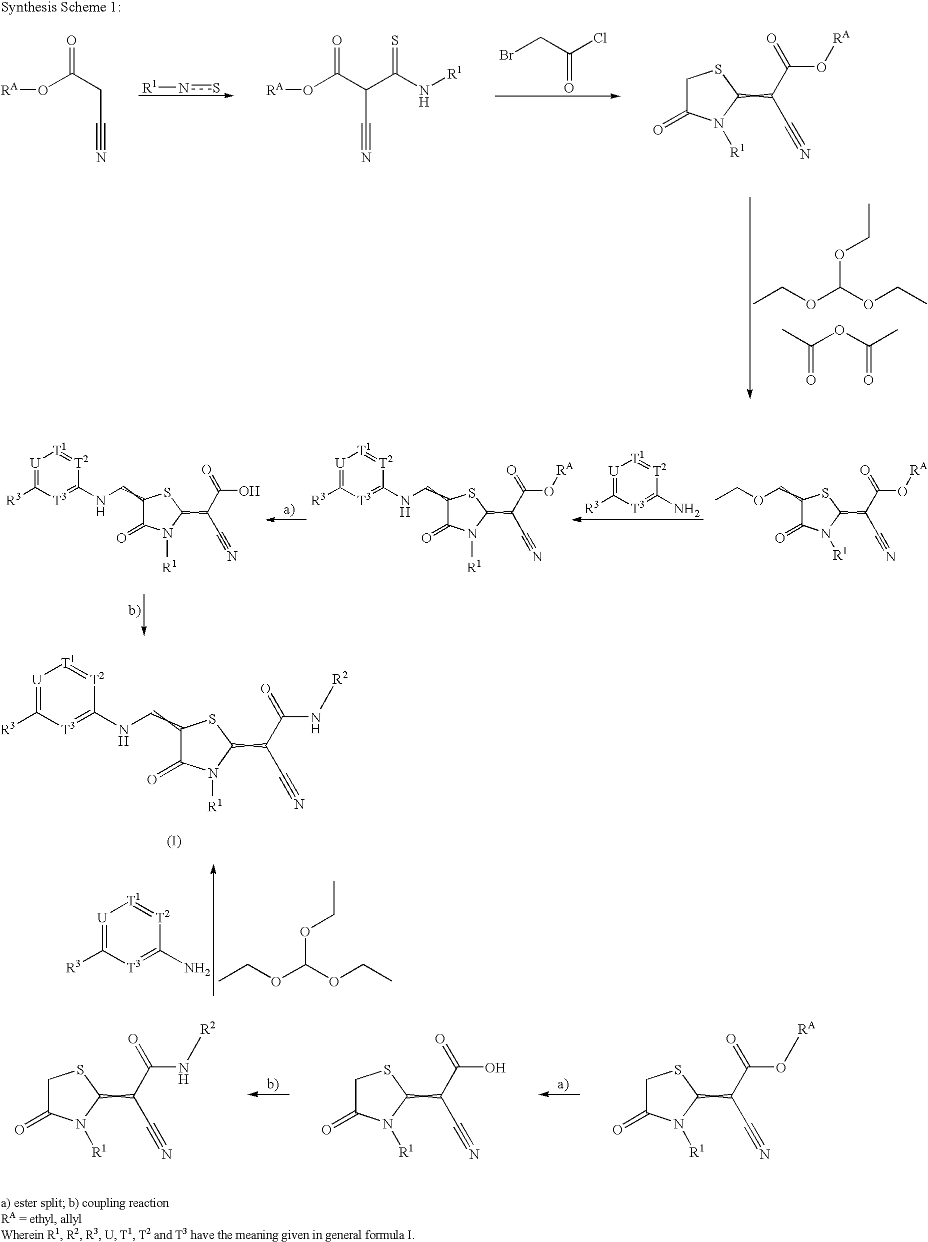

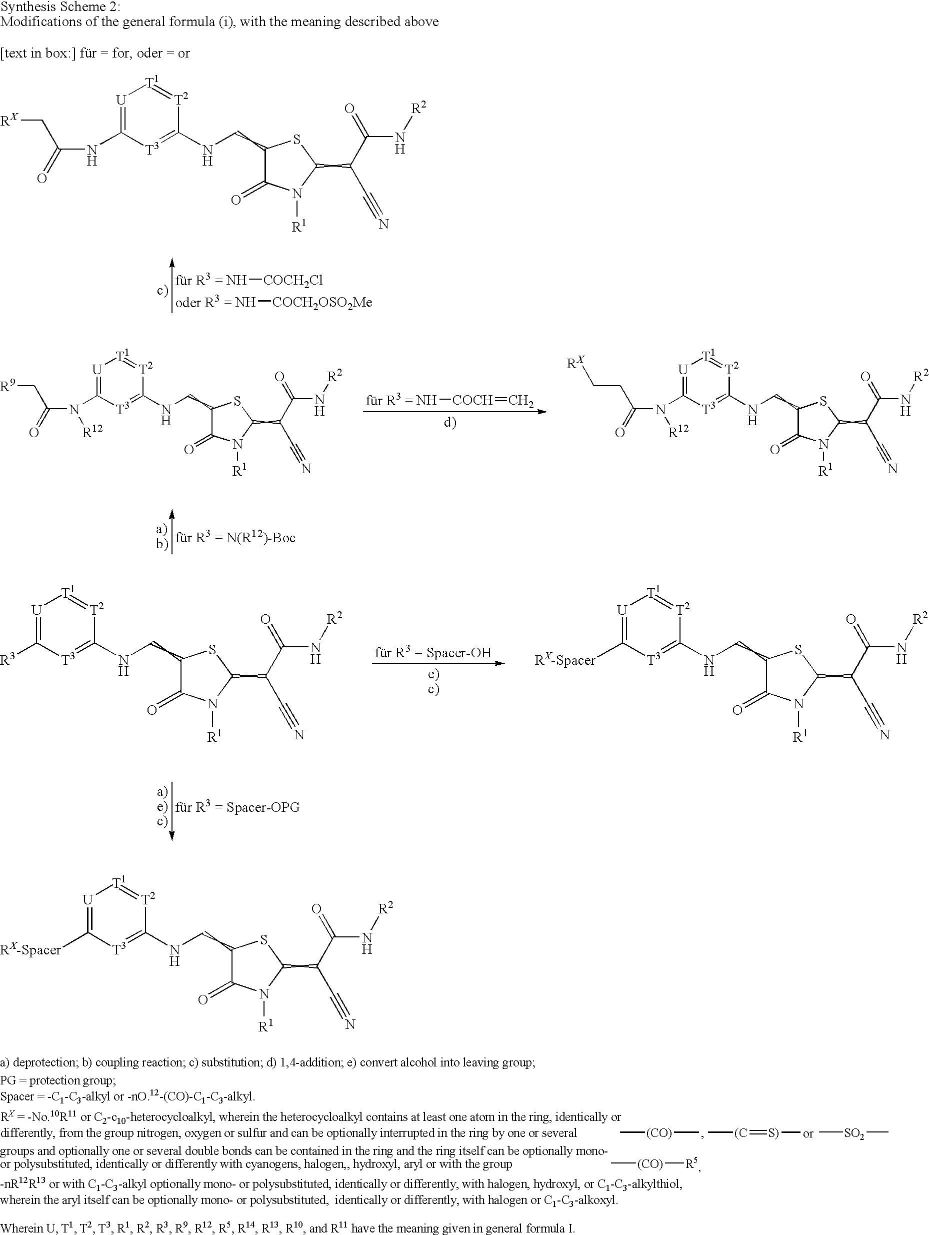

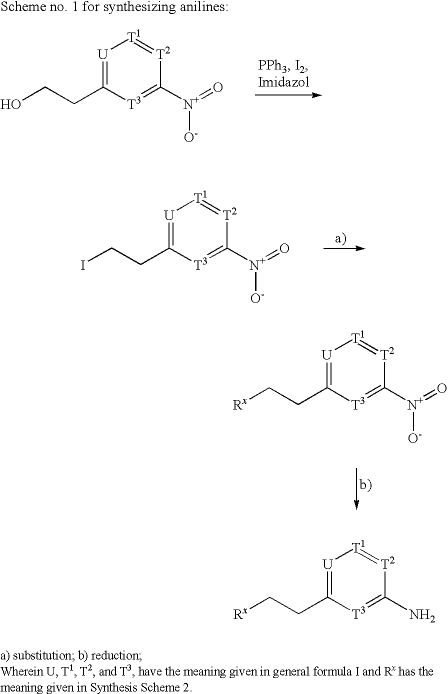

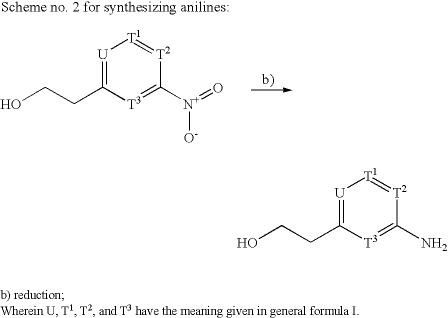

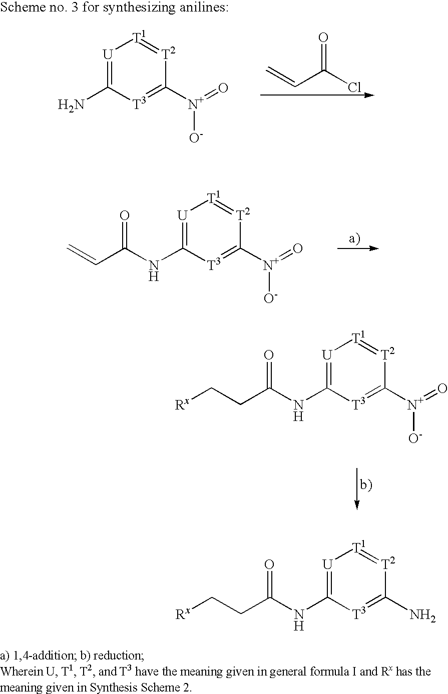

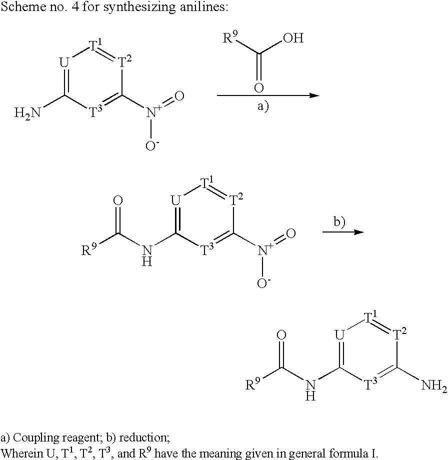

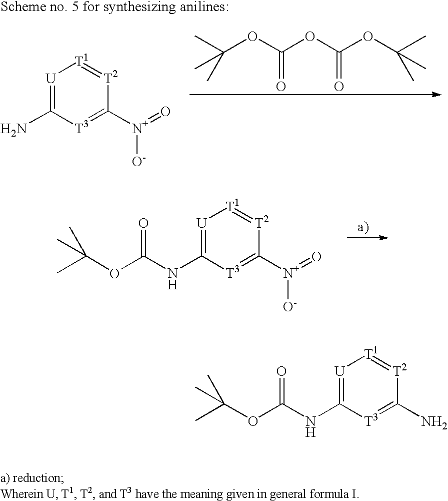

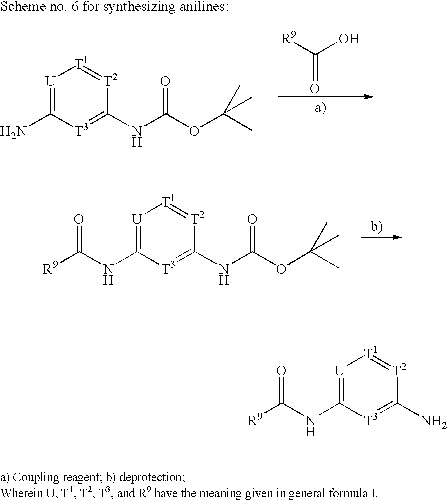

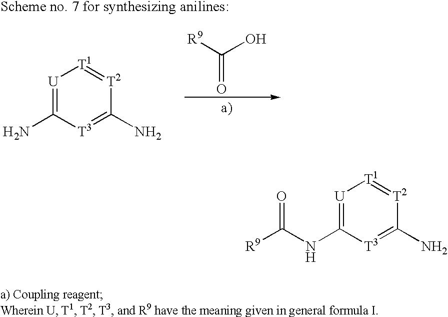

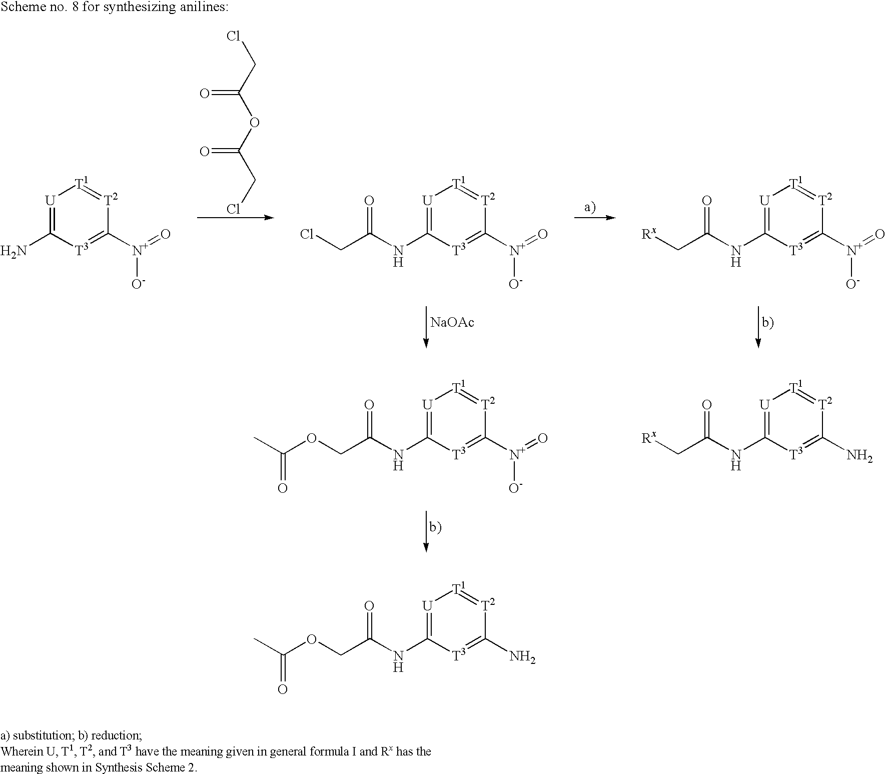

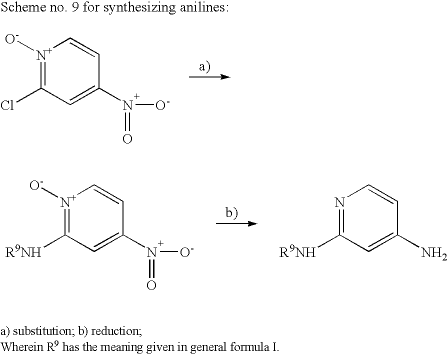

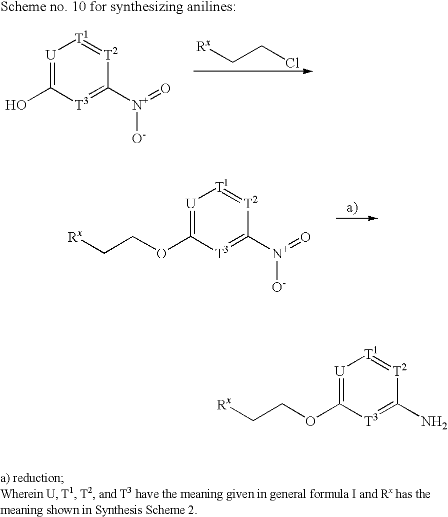

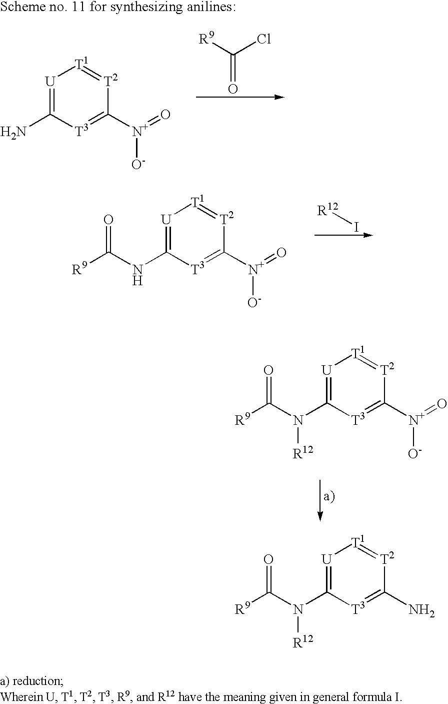

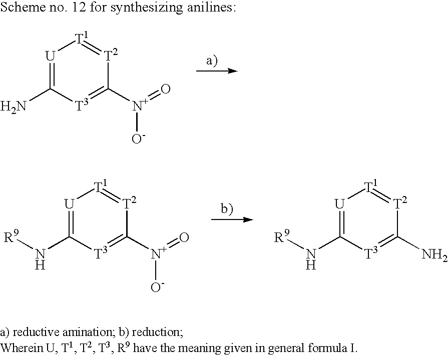

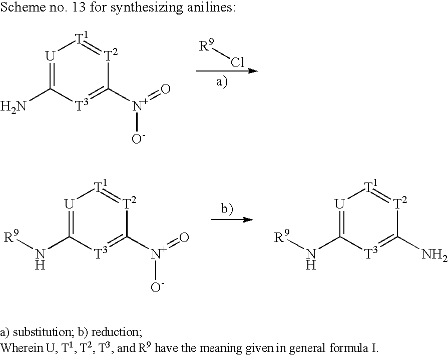

### 1. Synthesis of Aniline Components

### Intermediate INT1

### 1-(2-iodo-ethyl)-3-nitro-benzene

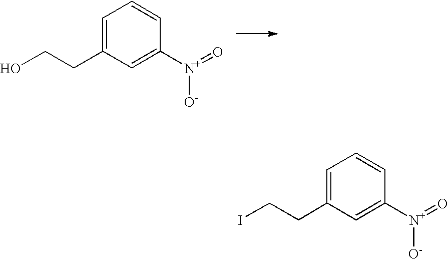

5 g 3-nitrophenyl ethanol, 9.4 g triphenylphosphine and 3.1 g imidazol are dissolved in 250 ml THF, mixed with 9.1 g iodine in portions and stirred for 15 hours at ambient temperature. The reaction mixture is mixed with ammonium chloride solution and extracted with dichloromethane. The organic phase is washed consecutively with sodiumthiosulfate solution and water and dried over sodium sulfate. After purification by chromatography on silica gel, 7.51 g of title compound is obtained.

1H-nMR (DMSO-d6): δ=3.31 (t, 2H); 3.41 (t, 2H); 7.46-7.60 (m, 2H); 8.09 (s, 1H); 8.16 (d, 1H); ppm.

### Intermediate INT2

### 1-[2-(3-nitro-phenyl)-ethyl]-pyrrolidine

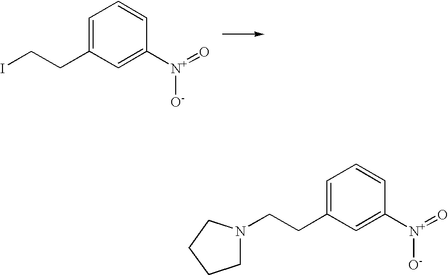

1.88 g of the compound described under INT1) is dissolved in 10 ml dimethylformamide, slowly mixed with 0.85 ml pyrrolidine and stirred for 15 hours at ambient temperature. The solution is condensed off in the high vacuum, the residue is incorporated into acetic acid ethylester and washed three times with water. The organic phase is dried over sodium sulfate. After purification by chromatography on silica gel, 350 g of title compound is obtained.

1H-nMR (CDCl3): δ=1.81 (m, 4H); 2.57 (m, 4H); 2.74 (t, 2H); 2.93 (t, 2H); 7.45 (t, 1H); 7.56 (d, 1H); 8.03-8.13 (m, 2H) ppm.

### Intermediate INT3

### 3-(2-pyrrolidin-1-yl-ethyl)-phenylamine

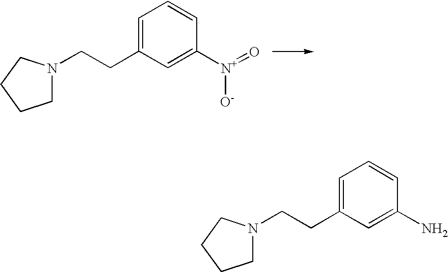

650 mg of the compound described under INT2) is dissolved in 250 ml ethanol and mixed (10%) with 130 mg palladium on carbon. It is stirred for 15 hours in a hydrogen atmosphere at ambient temperature. After filtration over kieselguhr and condensing off the solvent on the rotary evaporator, 540 mg of the title compound is obtained.

1H-nMR (DMSO-d6): δ=1.78 (m, 4H); 2.65 (t, 2H); 2.70-2.92 (m, 6H); 4.99 (s, 2H); 6.31-6.45 (m, 3H); 6.92 (t, 1H) ppm.

### Intermediate INT4

### N-(3-amino-phenyl)-2,2-dimethyl-propionamide

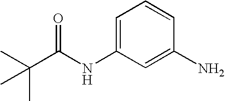

5.0 g of 1,3-diaminobenzol is dissolved in 50 ml dichloromethane and mixed at 0° C. with 24 ml diisopropylethylamine and 10.4 ml pivalic acid anhydride. It is stirred for 2 hours at 0° C. and 18 hours at ambient temperature. The reaction mixture is mixed with semisaturated sodiumhydrogencarbonate solution and extracted with acetic acid ethylester. The organic solution is washed with saturated sodiumchloride solution, dried over sodium sulfate, condensed and after being purified by chromatography on silica gel, 5.7 g of title compound is obtained.

1H-nMR (DMSO-d6): δ=1.20 (s, 9H); 4.98 (s, 2H); 6.24 (d, 1H); 6.70 (d, 1H); 6.83-6.96 (m, 2H) ppm.

### Intermediate INT5

### 1-(2-iodo-ethyl)-3-nitro-benzene

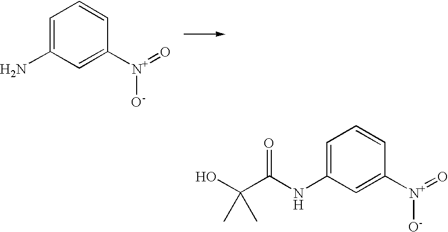

1.5 g of 2-hydroxy-2-methyl-propionic acid is mixed in 50 ml dimethylacetamide at −10° C. with 1.05 ml thionylchloride and stirred for 30 minutes at −10° C. A solution of 2 g 3-nitroaniline is dropped into 10 ml dimethylacetamide at −10° C. and stirred consecutively for one hour at −10° C., one hour at 0° C. and for 15 hours at ambient temperature. The solution is condensed off in the high vacuum, the residue is incorporated into a mixture of acetic acid ethylester and dichloromethane (1:3) and washed twice with semisaturated sodiumhydrogencarbonate solution. The organic phase is dried over sodium sulfate. After being purified by chromatography on silica gel, 2.42 g of title compound is obtained.

1H-nMR (CDCl3): δ=1.49 (s, 6H); 2.35 (s, 1H); 7.50 (t, 1H); 7.98 (d, 2H); 8.49 (s, 1H); 8.98 (s, b, 1H) ppm.

### Intermediate INT6

### N-(3-amino-phenyl)-2-hydroxy-2-methyl-propionamide

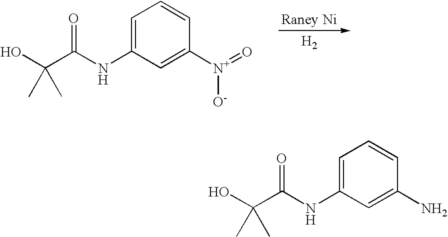

1.92 g of the compound described under INT5) is dissolved in 400 ml ethanol and mixed with 50 mg Raney nickel. It is stirred for 18 hours in a hydrogen atmosphere at ambient temperature. After filtration over kieselguhr and condensing off the solvent on the rotary evaporator, 1.9 g of title compound is obtained.

1H-nMR (CDCl3): δ=1.51 (s, 6H); 2.68 (s, 1H); 3.71 (s, b, 2H); 6.42 (d, 1H); 7.08 (t, 1H); 7.20 (s, 1H); 8.60 (s, b, 1H) ppm.

### Intermediate INT7

### 2-(2-methoxy-ethoxy)-n-(3-nitro-phenyl)-acetamide

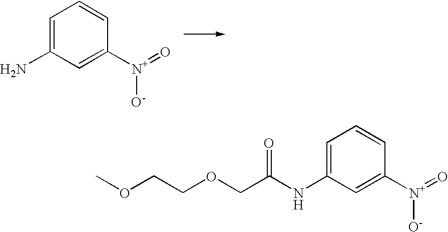

5 g (2-methoxyethoxy)-acetic acid is dissolved in 500 ml tetrahydrofurane. 9.7 ml triethylamine and 5.6 ml isobutylchloroformate is added at 0° C., and it is stirred for 30 minutes at 0° C. 5.0 g of 3-nitroaniline is added and it is stirred for another for 15 hours. The reaction mixture is mixed with semisaturated sodiumhydrogencarbonate solution and extracted with acetic acid ethylester. The organic solution is washed with saturated sodiumchloride solution, dried over sodium sulfate, condensed, and after being purified by chromatography on silica gel, 7.5 g of title compound is obtained.

1H-nMR (DMSO-d6): δ=3.30 (s, 3H); 3.53 (m, 2H); 3.70 (m, 2H); 4.04 (s, 1H); 7.62 (t, 1H); 7.93 (d, 1H); 8.02 (d, 1H); 8.69 (s, 1H); 10.20 (s, b, 1H) ppm.

### Intermediate INT8

### N-(3-amino-phenyl)-2-(2-methoxy-ethoxy)-acetamide

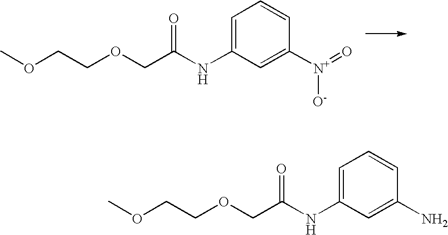

7.5 g of the compound described under INT7) is dissolved in 150 ml ethanol and mixed (10%) with 1.3 g palladium on carbon. It is stirred for 15 hours in a hydrogen atmosphere at ambient temperature. After filtration over kieselguhr and condensing off the solvent on the rotary evaporator, 6.5 g of title compound is obtained.

1H-nMR (DMSO-d6): δ=3.31 (s, 3H); 3.51 (m, 2H); 3.65 (m, 2H); 4.02 (s, 2H); 6.10 (s, 2H); 6.28 (d, 1H); 6.70 (d, 1H); 6.87-6.98 (m, 2H); 9.27 (s, 1H) ppm.

### Intermediate INT9

### N-(6-amino-pyridin-2-yl)-2,2-dimethyl-propionamide

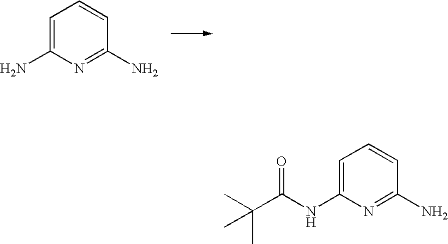

10 g of 2,6-diaminopyridine is dissolved in 150 ml tetrahydrofurane. 48 ml diisopropylethylamine and 20.8 ml pivalic acid anhydride is added and it is stirred for 15 hours at ambient temperature. The solvent is condensed off on the rotary evaporator. After purification by chromatography on silica gel, 10.6 g of title compound is obtained.

1H-nMR (DMSO-d6): δ=1.20 (s, 9H); 5.72 (s, 2H); 6.07 (d, 1H); 7.18 (d, 1H); 7.33 (t, 1H); 8.93 (s, 1H) ppm.

### Intermediate INT10

### N-(6-amino-pyridin-2-yl)-2-(2-methoxy-ethoxy)-acetamide

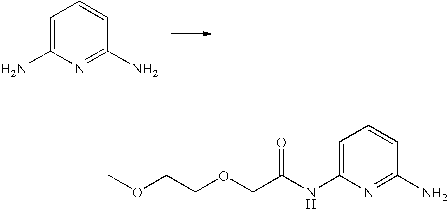

4.9 ml of (2-methoxyethoxy)-acetic acid is dissolved in 500 ml tetrahydrofurane. 9.7 ml triethylamine and 5.6 ml isobutylchloroformate is added at 0° C. and it is stirred for 30 minutes at 0° C. 3.96 g of 2,6-diaminopyridine is added and it is stirred for another 4 hours at ambient temperature. The reaction mixture is mixed with semisaturated sodiumhydrogencarbonate solution and extracted with acetic acid ethylester. The organic solution is washed with saturated sodiumchloride solution, dried over sodium sulfate, condensed and after being purified by chromatography on silica gel, 5.04 g of title compound is obtained.

1H-nMR (DMSO-d6): δ=3.31 (s, 3H); 3.50 (m, 2H); 3.67 (m, 2H); 4.07 (s, 2H); 5.88 (s, 2H); 6.19 (d, 1H); 7.21 (d, 1H); 7.36 (t, 1H); 9.13 (s, 1H) ppm.

### Intermediate INT11

### Ethyl-(4-nitro-1-oxy-pyridin-2-yl)-amine

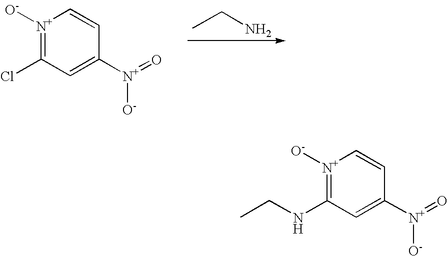

2.0 g of 2-chloro-4-nitro-pyridine 1-oxide is dissolved in 20 ml ethanol. 11.5 ml triethylamine is added and it is stirred for 4 hours under reflux. The solution is condensed off on the rotary evaporator. After purification by chromatography on silica gel, 1.5 g of title compound is obtained.

1H-nMR (DMSO-d6): δ=1.19 (t, 3H); 3.39 (pentuplet, 2H); 7.39 (dd, 1H); 7.47 (d, 1H); 7.64 (t, 1H); 8.35 (d, 1H) ppm.

### Intermediate INT12

### 4-amino-2-ethylamino-pyridine

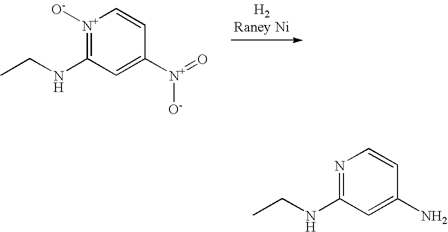

800 mg of the compound described under INT11) is dissolved in 50 ml ethanol and mixed with 50 mg Raney nickel. It is hydrated in a 3.5 bar hydrogen atmosphere at ambient temperature. After filtration over kieselguhr and condensing off the solvent on the rotary evaporator, 610 mg of title compound is obtained.

1H-nMR (DMSO-d6): δ=1.09 (t, 3H); 3.11 (m, 2H); 5.48 (s, 2H); 5.52 (d, 1H); 5.71 (t, 1H); 5.78 (dd, 1H); 7.49 (d, 1H) ppm.

### Intermediate INT13

### 2-chloro-n-(3-nitro-phenyl)-acetamide

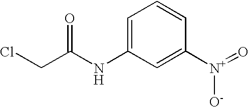

13.8 g of 3-nitroaniline is dissolved in 500 ml tetrahydrofurane. 30.5 ml triethylamine and 19.4 g chloroformic acid anhydride is added at 0° C. It is stirred for 12 hours at ambient temperature. The reaction mixture is mixed with semisaturated sodiumhydrogencarbonate solution and extracted with acetic acid ethylester. The organic solution is washed with saturated sodium chloride solution, dried over sodium sulfate, condensed and after being purified by chromatography on silica gel, 20.0 g of title compound is obtained.

1H-nMR (DMSO-d6): δ=4.31 (s, 2H); 7.64 (t, 1H); 7.89-8.00 (m, 2H); 8.61 (s, 1H); 10.79 (b, 1H) ppm.

### Intermediate INT14

### N-(3-nitro-phenyl)-2-piperidin-1-yl-acetamide

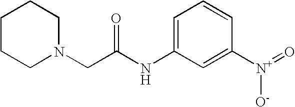

2.14 g of the compound described under INT13) is dissolved in 100 ml dimethylformamide. 2.0 ml triethylamine, 248 mg potassium iodide and 1.48 ml piperidine is added. It is stirred for 4 hours at ambient temperature. The reaction mixture is mixed with semisaturated sodiumhydrogencarbonate solution and extracted with acetic acid ethylester. The organic solution is washed with saturated sodium chloride solution, dried over sodium sulfate, condensed and after being purified by chromatography on silica gel, 1.97 g of title compound is obtained.

1H-nMR (DMSO-d6): δ=1.34-1.48 (m, 2H); 1.51-1.63 (m, 4H); 2.5 (m, 4H); 3.12 (s, 2H); 7.60 (t, 1H); 7.91 (d, 1H); 8.02 (d, 1H); 8.70 (s, 1H); 10.18 (s, 1H) ppm.

### Intermediate INT15

### Acetic acid (3-nitro-phenylcarbamoyl)-methyl ester

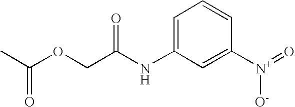

5.0 g of the compound described under INT13) is dissolved in 200 ml dimethylformamide. 19.1 g sodiumacetate and 350 mg potassium iodide is added. It is stirred for 24 hours at ambient temperature. The reaction mixture is mixed with water and extracted with acetic acid ethylester. The organic solution is washed three times with semisaturated sodiumchloride solution, dried over sodium sulfate, condensed and after being purified by chromatography on silica gel, 4.7 g of title compound is obtained.

1H-nMR (DMSO-d6): δ=2.14 (s, 3H); 4.70 (s, 2H); 7.62 (t, 1H); 7.87-7.98 (m, 2H); 8.60 (s, 1H); 10.57 (b, 1H) ppm.

### Intermediate INT16

### 4-[2-(2-methyl-5-nitro-phenoxy)-ethyl]-morpholine

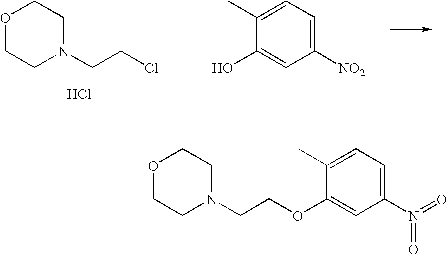

A suspension of 10 g 2-methyl-5-nitrophenol, 12 g 4-(2-chlorethyl)-morpholine and 27.1 g potassium carbonate is heated under reflux in 200 ml acetone for 15 hours. The batch is made free of solvent with the vacuum and the residue is incorporated into ethylacetate. It is extracted with NaOH aq. (1 n, 3×200 ml) and the united organic phases are dried over sodiumcarbonate, the solvent is distilled off on the rotary evaporator and a yield of 62% is obtained (10.9) of 4-[2-(2-methyl-5-nitro-phenoxy)-ethyl]-morpholine.

1H-nMR (300 MHz, CDCl₃): δ=2.30 (s, 3H); 2.61 (m, 4H); 2.86 (m, 2H); 3.71 (m, 4H); 4.20 (m, 2H); 7.22 (d, 1H); 7.68 (d, 1H); 7.75 (dd, 1H) ppm.

### Intermediate INT17

### 4-methyl-3-(2-morpholin-4-yl-ethoxy)-phenylamine

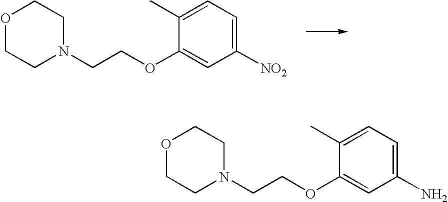

15.9 g of the compound described under INT16) and 2 g palladium on carbon is hydrated in 300 ml methanol at low pressure and ambient temperature. After hydrogen incorporation is completed, it is filtered off from the catalyst and the raw product is freed of solvent on the rotary evaporator. A quantitative yield of title compound is obtained. The raw product is used in the next step without further purification.

1H-nMR (300 MHz, CDCl₃): δ=2.10 (s, 3H); 2.62 (m, 4H); 2.85 (m, 2H); 3.77 (m, 4H); 4.10 (m, 2H); 6.21 (m, 2H); 6.90 (d, 1H) ppm.

The following compounds are produced according to the method described above.

| | | | Mol. weight/ | Educt/ |
|---|---|---|---|---|
| Example | | | MS (ESI) | synthesis |
| no. | Structure and name | ¹H-nMR | [M + 1]⁺ | analogous |
| INT18 | 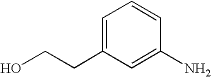 2-(3-amino-phenyl)-ethanol | (DMSO-d6, stored over K₂CO₃): δ = 2.06 (t, 1H); 3.55 (m, 2H); 4.60 (s, 1H); 4.91 (s, 2H); 6.25-6.50 (m, 3H); 6.91 (t, 1H) ppm. | | 3-nitro- phenyl- ethanol/ INT3 |
| INT19 | 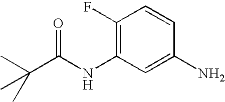 N-(5-amino-2-fluoro- phenyl)-2,2-dimethyl- propionamide | (DMSO-d6): δ = 1.20 (s, 9H); 4.93 (s, 2H); 6.29-6.38 (m, 1H); 6.70 (dd, 1H); 6.85 (dd, 1H); 8.77 (s, 1H) ppm. | | N-(5- nitro-2- fluoro- phenyl)- 2,2-dimethyl- propionamide/ INT3 |
| INT20 | 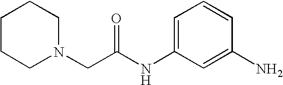 N-(3-amino-phenyl)-2- piperidin-1-yl-acetamide | (DMSO-d6): δ = 1.45 (m, 2H); 1.65 (m, 4H); 2.78 (m, 4H); 3.45 (s, 2H); 4.70-6.00 (b, 2H); 6.29 (d, 1H); 6.72 (d, 1H); 6.88-7.00 (m, 2H); 9.80 (s, 1 H) ppm. | | INT14/INT3 |
| INT21 | 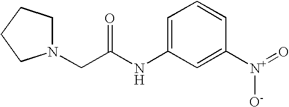 N-(3-ntro-phenyl)-2- pyrrolidin-1-yl-acetamide | (DMSO-d6): δ = 1.76 (m, 4H); 2.60 (m, 4H); 3.30 (s, 2H); 7.60 (t, 1H); 7.91 (d, 1H); 8.04 (d, 1H); 8.71 (s, 1H), 10.21 (s, b, 1H) ppm. | | INT13/INT14 |
| INT22 | 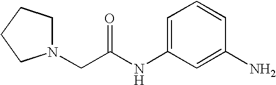 N-(3-amino-phenyl)-2- pyrrolidin-1-yl-acetamide | (DMSO-d6): δ = 1.85 (m, 4H); 3.00 (m, 4H); 3.71 (s, 2H); 4.70-5.55 (b, 2H); 6.28 (d, 1H); 6.71 (d, 1H); 6.87-6.97 (m, 2H); 9.88 (s, 1H) ppm. | | INT21/INT3 |
| INT23 | 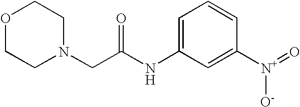 2-morpholin-4-yl-n-(3-nitro- phenyl)-acetamide | (DMSO-d6): δ = 2.51 (m, 4H); 3.19 (s, 2H); 3.55 (m, 4H); 7.60 (t, 1H); 7.92 (dd, 1H); 8.02 (dd, 1H); 8.79 (t, 1H); 10.25 (s, b, 1H) ppm. | | INT13/INT14 |
| INT24 | 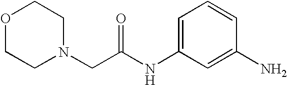 N-(3-amino-phenyl)-2- morpholin-4-yl-acetamide | (DMSO-d6): δ = 2.49 (m, 4H); 3.08 (s, 2H); 3.63 (m, 4H); 5.07 (s, 2H); 6.27 (d, 1H); 6.19 (d, 1H); 6.91 (t, 1H); 6.94 (s, 1H); 9.39 (s, 1H) ppm. | | INT23/INT3 |
| INT25 | 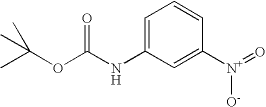 (3-nitro-phenyl)-carbamic acid tert-butyl ester | | MW: 238.25; MS (ESI) [M + 1]⁺: 239 | 3-nitro- aniline/ INT13 |
| INT26 | 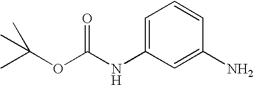 (3-amino-phenyl)-carbamic acid tert-butyl ester | | MW: 208.26; MS (ESI) [M + 1]⁺: 209 | INT25/INT3 |
| INT27 | 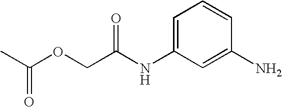 Acetic acid (3-amino- phenylcarbamoyl)-methyl ester | (DMSO-d6): δ = 2.11 (s, 3H); 4.60 (s, 2H); 5.08 (s, 2H); 6.28 (d, 1H); 6.67 (d, 1H); 6.83-6.97 (m, 2H); 9.75 (s, 1H) ppm. | | INT15/INT3 |
| INT28 | 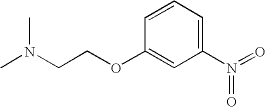 Dimethyl-[2-(3-nitro- phenoxy)-ethyl]-amine | | MW: 210.23; MS (ESI) [M + 1]⁺: 211 | 3-nitro- phenol/ INT16 |
| INT29 | 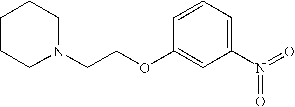 1-[2-(3-nitro-phenoxy)- ethyl]-piperidine | | MW: 250.30; MS (ESI) [M + 1]⁺: 251 | 3-nitro- phenol/ INT16 |
| INT30 | 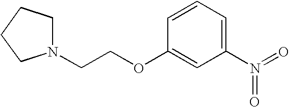 1-[2-(3-nitro-phenoxy)- ethyl]-pyrrolidine | | MW: 236.27; MS (ESI) [M + 1]⁺: 237 | 3-nitro- phenol/ INT16 |
| INT31 | 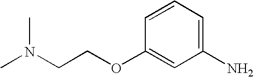 3-(2-dimethylamino-ethoxy)- phenylamine | | MW: 180.25; MS (ESI) [M + 1]⁺: 181 | INT28/INT3 |
| INT32 | 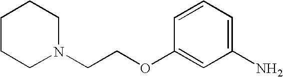 3-(2-piperidin-1-yl-ethoxy)- phenylamine | | MW: 220.32; MS (ESI) [M + 1]⁺: 221 | INT29/INT3 |
| INT33 | 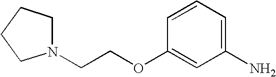 3-(2-pyrrolidin-1-yl-ethoxy)- phenylamine | | MW: 206.29; MS (ESI) [M + 1]⁺: 207 | INT30/INT3 |
| INT34 | 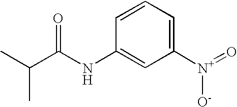 N-(3′-nitro-phenyl)- isobutyramide | | 208.218/209 | 3-nitro- anilin/ INT7 |
| INT35 | 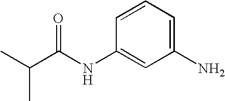 N-(3′-amino-phenyl)- isobutyramide | | 178.236/179 | INT34/INT8 |
| INT36 | 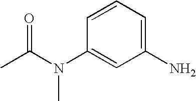 N-(3′-amino-phenyl)-n- methyl-acetamide | | 164.208/165 | INT49/INT8 |
| INT37 | 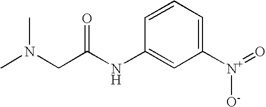 2-dimethylamino-n-(3′-nitro- phenyl)-acetamide | | 223.233/224 | INT13/INT14 |
| INT38 | 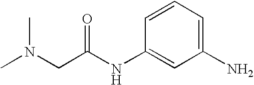 N-(3′-amino-phenyl)-2- dimethylamino-acetamide | | 193.250/194 | INT37/INT8 |
| INT39 | 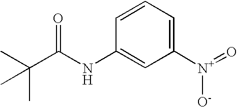 2,2-dimethyl-n-(3′-nitro- phenyl)-propionamide | | 222.245/223 | 3-nitro- anilin/ INT9 |
| INT40 | 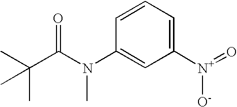 2,2,n-trimethyl-n-(3-nitro- phenyl)-propionamide | | 236.272/237 | INT39/INT-dK1 |
| INT41 | 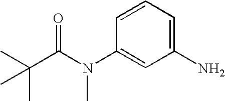 N-(3-amino-phenyl)-2,2,N- trimethyl-propionamide | | 206.290/207 | INT40/INT8 |
| INT43 | 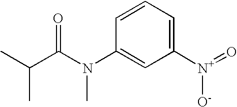 N-methyl-n-(3′-nitro- phenyl)-isobutyramide | | 222.245/223 | INT34/INT49 |
| INT44 | 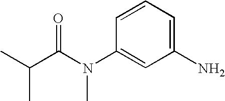 N-(3-amino-phenyl)-n- methyl-isobutyramide | | 192.263/193 | INT43/INT8 |
| INT45 | 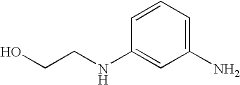 2-(3-amino-phenylamino)- ethanol | | 152.197/153 | INT50/INT8 |
| INT46 | 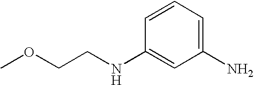 N-(2-methoxy-ethyl)- benzen-1,3-diamine | | 166.224/167 | INT51/INT8 |
| INT47 | 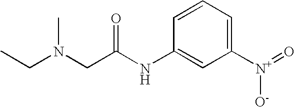 2-(ethyl-methyl-amino)-n- (3′-nitro-phenyl)-acetamide | | 237.260/238 | INT13/INT14 |
| INT48 | 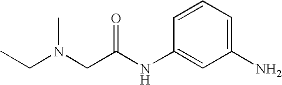 N-(3-amino-phenyl)-2- (ethyl-methyl-amino)- acetamide | | 207.277/208 | INT47/INT8 |

### Intermediate INT49

### N-methyl-n-(3-nitro-phenyl)-acetamide

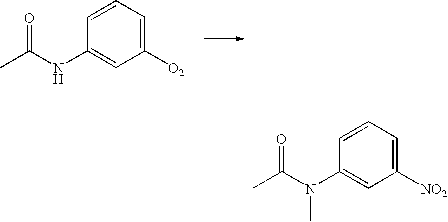

0.43 g of sodiumhydride (60% suspension in mineral oil) is washed in a round-bottomed flask in protective gas with n-hexane (3×) and suspended in a little THF. A solution of 1.3 g 3-nitroacetanilide in 15 ml THF is dripped into that suspension. After the formation of gas has abated, 4.5 ml methyliodide is dripped into the reaction mixture. It is stirred for 2 hours at ambient temperature. The solvent is then distilled off to the greatest extent possible. Any sodium hydride that is left unconverted is broken down by adding a little water. The residue yielded is incorporated into ethylacetate. The organic phase is consecutively washed with water and saturated sodiumchloride solution and dried over magnesium sulfate. The oil yielded after evaporation is purified in silica gel. 1.23 g of title compound was yielded as a light-yellow oil.

¹H-nMR (CDCl₃): δ=1.93 (s, 3H); 3.31 (s, 3H); 7.56-7.64 (m, 2H); 8.09 (s, 1H); 8.18-8.20 (m, 1H) ppm. MS (ESI) [M+1]⁺: 195.

### Intermediate INT50

### 2-(3-nitro-phenylamino)-ethanol

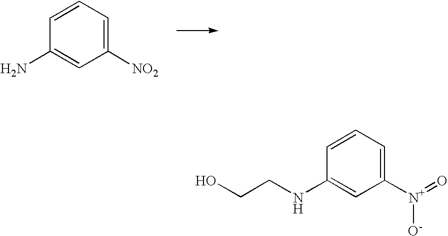

195 mg glycoaldehyde, 195 mg sodium cyanoborohydride and 0.08 ml glacial acetate is added to a solution of 200 mg 3-nitroaniline in 10 ml methanol cooled to 0° C. It is stirred for 5 hours at ambient temperature. For conversion it is mixed with 150 ml sodiumhydrogencarbonate solution and extracted with ethylacetate. The organic phase is washed with saturated sodiumchloride solution and dried over magnesium sulfate. The oil yielded after evaporation is purified on silica gel. 224 mg of title compound was yielded as orange crystals.

¹H-nMR (DMSO-d₆): δ=3.15 (q, 2H); 3.56 (q, 2H); 4.76 (t, 1H); 6.39 (t, 1H); 6.97-6.99 (m, 1H); 7.28-7.34 (m, 3H) ppm. MS (ESI) [M+1]⁺: 183.

### Intermediate INT51

### N-(2-methoxy-ethyl)-benzen-1,3-diamine

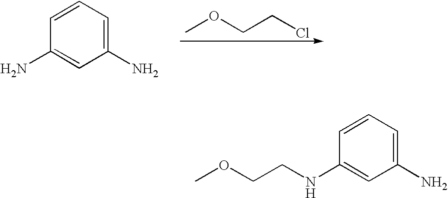

A mixture comprising 5 g 1,3-phenylenediamine, 4.2 ml 2-methoxyethylchloride, 4.9 g sodiumcarbonate (anhydride) and 30 ml water is boiled for 12 hours under reflux. It is then diluted with water (600 ml) and filtered. The filtrate is extracted with ethylacetate. The organic phase is washed consecutively with water and saturated sodiumchloride solution and dried over magnesiumsulfate. The oil yielded after evaporation is purified on silica gel. 1.85 g of title compound was yielded as oil.

1H-nMR (DMSO-d6): δ=3.09 (q, 2H); 3.25 (s, 3H); 3.43 (t, 2H); 4.68 (s, 2H); 5.10 (t, 1H); 5.78-5.81 (m, 3H); 6.69 (t, 1H) ppm. MS (ESI) [M+1]+: 167.

### Intermediate INT52

### 2-(3-nitro-phenyl)-oxirane

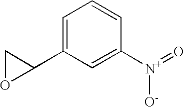

10 g of 2-bromo-1-(3-nitro-phenyl)-ethanone is dissolved in 200 ml ethanol, mixed with 1.55 g sodiumborohydride and stirred for 1 hour at ambient temperature. 2.1 g potassium hydroxide is added and it is stirred for another for 15 hours at ambient temperature. 1000 ml acetic acid ethylester is added and washed twice with 300 ml semisaturated ammonium chloride solution and once with 100 ml water. The organic phase is dried over sodium sulfate. After purification by chromatography on silica gel, 7.48 g of title compound is obtained. ¹H NMR (CDCl₃): δ=2.79 (dd, 1H); 3.19 (dd, 1H); 3.93 (dd, 1H); 7.50 (t, 1H); 7.60 (d, 1H); 8.08-8.16 (m, 2H) ppm.

### Intermediate INT53

### 1-(3-nitro-phenyl)-2-piperidin-1-yl-ethanol

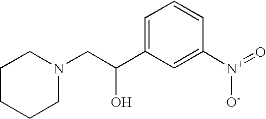

1.68 g of the compound described under INT52 was dissolved in 10 ml tetrahydrofurane and mixed with 1.5 ml piperidine and stirred under reflux for 15 hours. The solvent is distilled off on the rotary evaporator, and after being purified by chromatography on silica gel, 1.4 g of title compound is obtained. P ¹H NMR (CDCl₃): δ=1.40-1.80 (m, 6H); 2.23-2.49 (m, 3H); 2.59 (dd, 1H); 2.71 (b, 2H); 4.35 (b, 1H); 4.80 (dd, 1H); 7.51 (t, 1H); 7.73 (d, 1H); 8.13 (d, 1H); 8.28 (s, 1H) ppm.

### Intermediate INT54

### 1-(3-amino-phenyl)-2-piperidin-1-yl-ethanol

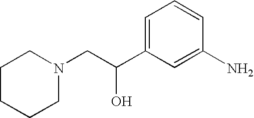

2.0 g of the compound described under INT53 is dissolved in 250 ml ethanol and mixed (10%) with 200 mg palladium on carbon. It is stirred for 15 hours in a hydrogen atmosphere at ambient temperature. After filtration over kieselguhr and condensing off the solvent on the rotary evaporator, 1.76 g of title compound is obtained.

¹H NMR (CDCl₃): δ=1.40-1.70 (m, 6H); 2.28-2.55 (m, 4H); 2.58-2.77 (m, 2H); 3.65 (b, 2H); 4.63 (dd, 1H); 6.52-6.62 (m, 1H); 6.72 (d, 1H); 6.75 (s, 1H); 7.11 (t, 1H) ppm.

### Intermediate INT55

### 1-(3-nitro-phenyl)-2-(4aR,8aS)-octahydro-isoquinolin-2-yl-ethanol (Diastereomers mixture)

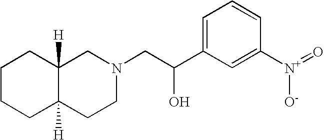

5.0 g of the compound described under INT52 is dissolved in 50 ml tetrahydrofurane and mixed with 7.3 g trans-decahydroisochinolin and stirred for 20 hours under reflux. The solvent is distilled off on the rotary evaporator, and after being purified by chromatography on silica gel, 5.75 g of title compound is obtained.

¹H NMR (CDCl₃): δ=0.72-1.45 (m, 7H); 1.45-1.85 (m, 6H); 1.95-3.20 (m, 5H); 4.43 (b, 1H); 4.75-4.86 (m, 1H); 7.51 (t, 1H); 7.72 (d, 1H); 8.13 (d, 1H); 8.25 (s, 1H) ppm.

### Intermediate INT56

### Acetic acid (4aR,8aS)-1-(3-nitro-phenyl)-2-octahydro-isoquinolin-2-yl-ethyl ester

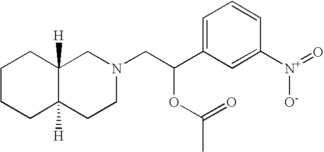

5.75 g of the compound described under INT55 is dissolved in 100 ml tetrahydrofurane and mixed at 0° C. with 5.4 ml triethylamine and 3.6 ml acetanehydride and then stirred for 48 hours at ambient temperature. Half the solvent is distilled off on the rotary evaporator, 100 ml semisaturated sodiumhydrogencarbonate solution is added and it is extracted three times with 150 ml dichlormethane each time. The united organic phases are dried over sodium sulfate. After purification by chromatography on silica gel and then recrystallization, 4.07 g of title compound is obtained.

¹H NMR (CDCl₃; main isomer): δ=0.72-1.05 (m, 3H); 1.06-1.35 (m, 4H); 1.40-1.89 (m, 6H); 2.00-2.22 (m, 4H); 2.55 (dd, 1H); 2.64-2.96 (m, 3H); 5.97 (dd, 1H); 7.51 (t, 1H); 7.68 (d, 1H); 8.14 (d, 1H); 8.22 (s, 1H) ppm.

### Intermediate INT57

### 3-[(4aR,8aS)-2-(octahydro-isoquinolin-2-yl)-ethyl]-phenylamine

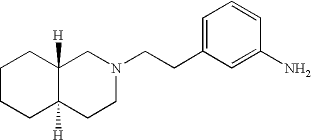

4.07 g of the compound described under INT56) is dissolved in 400 ml acetic acid ethylester and 100 ml glacial acetate and mixed (10%) with 400 mg palladium on carbon. It is hydrated for 15 hours under 100 bar hydrogen at ambient temperature. Another 1000 mg palladium on carbon is added (10%) and hydrated for another 15 hours under 100 bar hydrogen at ambient temperature. Half the solvent is distilled off on the rotary evaporator, approx. 1 L of 2 normal sodium hydroxide solution is added until the solution has a pH von 9.5. The solution is consecutively extracted with 300 ml acetic acid ethylester and with 500 ml of a mixture of chloroform and methanol (10:1). The united organic phases are washed with water (100 ml) and saturated table salt solution (100 ml) and dried over sodium sulfate. After filtering and condensing off the solvent on the rotary evaporator, 2.57 g of title compound is obtained.

¹H NMR (CDCl₃): δ=0.69-1.03 (m, 3H); 1.03-1.33 (m, 4H); 1.39-1.73 (m, 6H); 1.86. 2.00 (m, 1H); 2.41-2.53 (m, 2H); 2.61-2.71 (m, 2H); 2.75-2.83 (m, 1H); 2.88-3.00 (m, 1H); 3.37-3.70 (b, 2H); 6.40-6.50 (m, 2H); 6.54 (d, 1H); 7.00 (t, 1H) ppm.

### Intermediate INT58

### 2-chloro-n-(2-fluoro-5-nitro-phenyl)-acetamide

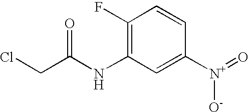

10 g 2-fluoro-5-nitro-phenylamine is dissolved in 330 ml tetrahydrofurane mixed at 0° C. with 19.5 ml triethylamine, 0.5 ml pyridine and 5.6 ml chloracetylchloride and then stirred for 24 hours at ambient temperature. The solvent is distilled off on the rotary evaporator, 1 L of acetic acid ethylester is added and washed with 200 ml semisaturated sodiumhydrogencarbonate solution. The organic phases are dried over sodium sulfate. After purification by chromatography on silica gel, 5.4 g of title compound is obtained.

¹H NMR (CDCl₃): δ=4.27 (s, 2H); 7.21-7.38 (m, 1H); 7.97-8.31 (m, 1H); 8.66 (s, b, 1H); 9.19-9.32 (m, 1H) ppm.

### Intermediate INT59

### N-(2-fluoro-5-nitro-phenyl)-2-morpholin-4-yl-acetamide

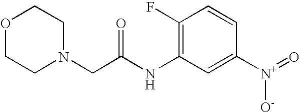

3.0 g of the compound described under INT58 is dissolved in 50 ml dimethylformamide, mixed with 2.68 ml triethylamine, 330 mg potassium iodide and 1.18 ml 4,4-morpholine and stirred for 15 hours at ambient temperature. The solvent is distilled off on the rotary evaporator, 500 ml acetic acid ethylester is added and then it is washed with 50 ml water and twice with 50 ml semisaturated sodiumhydrogencarbonate solution. The organic phase is dried over sodium sulfate. After purification by chromatography on silica gel, 2.7 g of title compound is obtained.

¹H NMR (CDCl₃): δ=2.66 (t, 4H); 3.23 (s, 2H); 3.79 (t, 4H); 7.18-7.33 (m, 1H); 7.92-8.05 (m, 1H); 9.27-9.39 (m, 1H); 9.73 (s, b, 1H) ppm.

### Intermediate INT60

### N-(5-amino-2-fluoro-phenyl)-2-morpholin-4-yl-acetamide

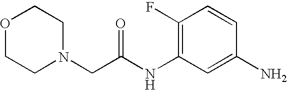

2.7 g of the compound described under INT59 is dissolved in 500 ml ethanol and mixed (10%) with 270 mg palladium on carbon. It is stirred for 15 hours in a hydrogen atmosphere at ambient temperature. After filtration over kieselguhr and condensing off the solvent on the rotary evaporator, 2.4 g of title compound is obtained.

¹H NMR (CDCl₃): δ=2.62 (t, 4H); 3.15 (s, 2H); 3.35-3.70 (b, 2H); 3.77 (t, 4H); 6.25-6.39 (m, 1H); 6.81-6.95 (m, 1H); 7.70-7.84 (m, 1H); 9.44 (s, b, 1H) ppm.

### Intermediate INT61

### 2-(4,4-difluoro-piperidin-1-yl)-n-(2-fluoro-5-nitro-phenyl)-acetamide

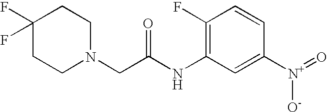

1.41 g of the compound described under INT58 is dissolved in 25 ml dimethylformamide, mixed with 1.26 ml triethylamine, 155 mg potassium iodide and 1.0 g 4,4-difluoropiperidine and stirred for 15 hours at ambient temperature. The solvent is distilled off on the rotary evaporator, 500 ml of a mixture of dichloromethane and methanol (100:1) is added and then it is washed twice each time with 50 ml semisaturated sodiumhydrogencarbonate solution. The organic phase is dried over sodium sulfate. After purification by chromatography on silica gel, 1.1 g of title compound is obtained.

¹H NMR (CDCl₃): δ=2.00-2.21 (m, 4H); 2.78 (t, 4H); 3.28 (s, 2H); 7.18-7.34 (m, 1H); 7.91-8.52 (m, 1H); 9.25-9.38 (m, 1H); 9.62 (s, b, 1H) ppm.

### Intermediate INT62

### N-(5-amino-2-fluoro-phenyl)-2-(4,4-difluoro-piperidin-1-yl)-acetamide

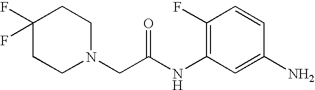

1.1 g of the compound described under INT61 is dissolved in 200 ml ethanol and mixed (10%) with 110 mg palladium on carbon. It is stirred for 15 hours in a hydrogen atmosphere at ambient temperature. After filtration over kieselguhr and condensing off the solvent on the rotary evaporator, 0.99 g of title compound is obtained.

¹H NMR (CDCl₃): δ=1.93-2.20 (m, 4H); 2.73 (t, 4H); 3.20 (s, 2H); 3.60 (b, 2H); 6.24-6.44 (m, 1H); 6.87 (t, 1H); 7.65-7.85 (m, 1H); 9.36 (s, b, 1H) ppm.

### Intermediate INT63

### (5-bromo-2-chloro-pyrimidin-4-yl)-(2-methoxy-ethyl)-amine

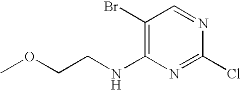

5.0 g 5-bromo-2,4-dichloropyrimidine is dissolved in 100 ml acetonitrile, mixed with 5.2 ml triethylamine and 1.85 ml 2-methoxyethylamine and stirred for 15 hours at ambient temperature. 100 ml acetic acid ethylester is added and then it is washed twice with 50 ml water and twice with 50 ml saturated sodiumchloride solution. The organic phase is dried over sodium sulfate. After purification by chromatography on silica gel, 4.97 g of title compound is obtained.

¹H NMR (CDCl₃): δ=3.46 (s, 3H); 3.62 (t, 2H); 3.77 (m, 2H); 5.98 (s, b, 1H); 8.18 (s, 1H) ppm.

### Intermediate INT64

### 5-bromo-n*4*-(2-methoxy-ethyl)-pyrimidine-2,4-diamine

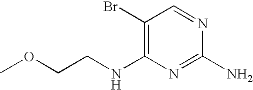

2.97 g of the compound described under INT63 is dissolved in 80 ml methanol. The solution is saturated at 8 bar with ammoniac and the closed autoclave is stirred for 20 hours at 80° C. The solvent is distilled off on the rotary evaporator. The residue is mixed with 10 ml methanol, incorporated into 100 ml chloroform and washed twice with 20 ml water. After purification by chromatography on silica gel, 1.4 g of title compound is obtained.

¹H NMR (CDCl₃): δ=3.39 (s, 3H); 3.54 (t, 2H); 3.61 (m, 2H); 4.82 (s, b, 2H); 5.54 (s, b, 1H); 7.86 (s, 1H) ppm.

### Intermediate INT65

### N*4*-(2-methoxy-ethyl)-pyrimidine-2,4-diamine

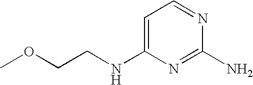

1.1 g of the compound described under INT64 is dissolved in 250 ml ethanol and mixed (10%) with 110 mg palladium on carbon. It is stirred for 15 hours in a hydrogen atmosphere at ambient temperature. After filtration over kieselguhr and condensing off the solvent on the rotary evaporator, 0.99 g of title compound is obtained as HBr salt.

¹H NMR (DMSO-d₆, stored over K₂CO₃): δ=3.27 (s, 3H); 3.43-3.58 (m, 4H); 6.12 (d, 1H); 7.64 (d, 1H); 7.73 (s, b, 2H); 8.81 (s, b, 1H); 11.57 (s, b, 1H) ppm.

### Intermediate INT66

### (R)-2-(5-bromo-2-chloro-pyrimidin-4-ylamino)-3-methyl-butan-1-ol

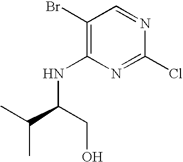

Analogous to the production of intermediate INT63, the title compound is obtained starting from 5-bromo-2,4-dichloropyrimidine and (R)-2-amino-3-methyl-butan-1-ol.

Mol. weight/MS (ESI) [M+1]⁺: 294.58/294; 296 (100%); 298.

### Intermediate INT67

### (R)-2-(2-amino-5-bromo-pyrimidin-4-ylamino)-3-methyl-butan-1-ol

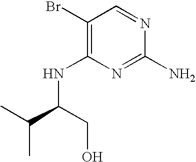

1.0 g of the compound described under INT66 is dissolved in 100 ml methanol. The solvent is saturated at 8 bar with ammoniac and the closed autoclave is stirred for 20 hours at 80° C. The solvent is distilled off on the rotary evaporator. The residue is mixed with 5 ml methanol, incorporated into 50 ml chloroform and washed twice with 20 ml water. After purification by chromatography on silica gel, 640 mg of title compound is obtained.

¹H NMR (DMSO-d6, stored over K₂CO₃): δ=0.90-1.04 (m, 6H); 1.91-2.08 (m, 1H); 3.00 (s, b, 1H); 3.70 (dd, 1H); 3.80 (dd, 1H); 3.95 (m, 1H); 4.89 (s, 2H); 5.33 (d, 1H); 7.89 (s, 1H) ppm.

### Intermediate INT68

### (6-bromo-pyridin-2-yl)-difluoro-acetic acid ethyl ester

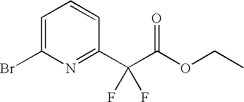

6.75 g 2,6-dibromopyridine is dissolved in 40 ml dimethylsulfoxide. 4.1 g of copper powder and 7.51 g ethylbromodifluoroacetate is added and stirred for 4 hours at 50° C. The reaction mixture is mixed with 200 ml acetic acid ethylester and 200 ml 1.3 molar potassium hydrogenphosphate solution and stirred for 30 minutes at ambient temperature. The solid substance is filtered off, the organic phase is separated off, it is washed three times consecutively with 50 ml semisaturated table salt solution and dried over sodium sulfate. After purification by chromatography on silica gel, 4.1 g of title compound is obtained.

¹H NMR (DMSO-d6, stored over K₂CO₃): δ=1.24 (t, 3H); 4.38 (q, 2H); 7.88-7.97 (m, 2H); 8.03 (t, 1H) ppm.

### Intermediate INT69

### 2-(6-bromo-pyridin-2-yl)-2,2-difluoro-ethanol

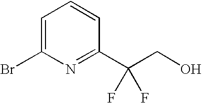

7.75 g of the compound described under INT68 is dissolved in 130 ml ethanol, mixed at 0° C. with 785 mg sodium borohydride and stirred for 4 hours at ambient temperature. 15 ml of 2 molar salt acid is added subject to the cooling of an ice bath. It is stirred for 10 minutes at ambient temperature and brought up to pH 10 with caustic soda. The reaction mixture is mixed with 500 ml dichloromethane and 100 ml semisaturated table salt solution, the organic phase is separated off and dried over sodium sulfate. After purification by filtration through silica gel, 6.3 g of title compound is obtained.

¹H NMR (DMSO-d6, stored over K₂CO₃): δ=3.93 (t, 2H); 5.59 (s, 1H); 7.70 (d, 1H); 7.79 (d, 1H); 7.90 (t, 1H) ppm.

### Intermediate INT70

### 2-bromo-6-[2-(tert-butyl-dimethyl-silanyloxy)-1,1-difluoro-ethyl]-pyridine

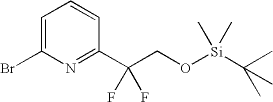

6.9 g of the compound described under INT69 is dissolved in 60 ml dimethylformamide, mixed with 3.77 g imidazol and 5.27 g tert.-butyldimethylsilylchloride and stirred for 15 hours at ambient temperature. 300 ml semisaturated sodiumhydrogencarbonate solution is added and extracted three times with 150 ml acetic acid ethylester. The united organic phases are dried over sodium sulfate. After purification by filtration through silica gel, 9.2 g of title compound is obtained.

¹H NMR (DMSO-d6, stored over K₂CO₃): δ=−0.07 (s, 6H); 0.70 (s, 9H); 4.16 (t, 2H); 7.72 (d, 1H); 7.80 (d, 1H); 7.91 (t, 1H) ppm.

### Intermediate INT71

### {6-[2-(tert-butyl-dimethyl-silanyloxy)-1,1-difluoro-ethyl]-pyridin-2-yl}-(2,4-dimethoxy-benzyl)-amine

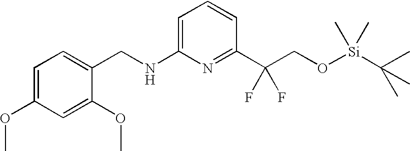

2.5 g of the compound described under INT70 is dissolved in 25 ml dioxane, mixed with 2.7 ml of 2,4-dimethylbenzylamine, 168 mg palladium acetate, 218 mg BINAP and 950 mg sodium-tert-butylate and stirred for 3 hours at 80° C. 100 ml water is added and extracted three times with 50 ml acetic acid ethylester. The united organic phases are dried over sodium sulfate. After purification by chromatography on silica gel, 2.3 g of title compound is obtained.

¹H NMR (DMSO-d6, stored over K₂CO₃): δ=−0.07 (s, 6H); 0.75 (s, 9H); 3.69 (s, 3H); 3.77 (s, 3H); 4.06 (t, 2H); 4.30 (d, 2H); 6.39 (d, 2H); 6.48-6.58 (m, 2H); 6.69 (d, 1H); 6.97 (t, 1H); 7.20 (d, 1H); 7.41 (t, 1H) ppm.

### Intermediate INT72

### 2-[6-(2,4-dimethoxy-benzylamino)-pyridin-2-yl]-2,2-difluoro-ethanol

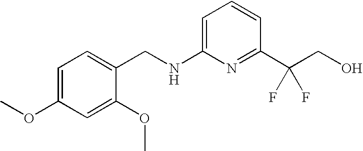

2.3 g of the compound described under INT71 is dissolved in 100 ml tetrahydrofurane and mixed with 13 ml of a 1 molar solution of tetrabutylammoniumfluoride in tetrahydrofurane and stirred for 1 hour at ambient temperature. 100 ml semisaturated sodiumhydrogencarbonate solution is added and extracted three times with 100 ml acetic acid ethylester. The united organic phases are dried over sodium sulfate. After purification by chromatography on silica gel, 1.42 g of title compound is obtained.

¹H NMR (DMSO-d6, stored over K₂CO₃): δ=3.70 (s, 3H); 3.77 (s, 3H); 3.87 (t, 2H); 4.30 (d, 2H); 5.37 (s, b, 1H); 6.41 (d, 1H); 6.50-6.59 (m, 2H); 6.70 (d, 1H); 6.95 (t, 1H); 7.13 (d, 1H); 7.41 (t, 1H) ppm.

### Intermediate INT73

### Methane sulfonic acid 2-[6-(2,4-dimethoxy-benzylamino)-pyridin-2-yl]-2,2-difluoro-ethyl ester

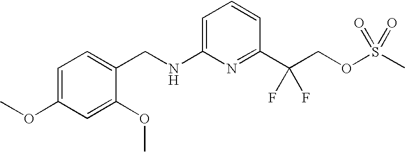

1.37 g of the compound described under INT72 is dissolved in 100 ml tetrahydrofurane and mixed at 0° C. with 1.47 ml triethylamine and 0.49 ml methanesulfonic acid chloride and then stirred for 2 hours at ambient temperature. 100 ml water is added and extraction with 50 ml acetic acid ethylester occurs. The united organic phases are dried over sodium sulfate. After purification by chromatography on silica gel, 1.56 g of title compound is obtained.

¹H NMR (DMSO-d6, stored over K₂CO₃): δ=3.19 (s, 3H); 3.70 (s, 3H); 3.77 (s, 3H); 4.31 (d, 2H); 4.79 (t, 2H); 6.41 (d, 1H); 6.52 (s, 1H); 6.62 (d, 1H); 6.79 (d, 1H); 7.08-7.19 (m, 2H); 7.49 (t, 1H) ppm.

### Intermediate INT74

### [6-(1,1-difluoro-2-pyrrolidin-1-yl-ethyl)-pyridin-2-yl]-(2,4-dimethoxy-benzyl)-amine

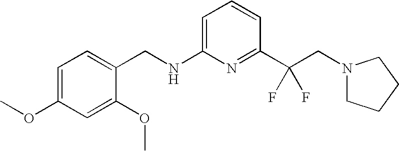

2.0 g of the compound described under INT73 is dissolved in 40 ml dimethylformamide, mixed with 1.38 g potassium carbonate, 120 mg potassium iodide and 2.1 ml pyrrolidine and then stirred for 24 hours at 120° C. 200 ml acetic acid ethylester is added and then washed three times with water (50 ml) and three times with 50 ml semisaturated sodiumchloride solution. The organic phase is dried over sodium sulfate. After purification by chromatography on silica gel, 1.35 g of title compound is obtained.

¹H NMR (DMSO-d6, stored over K₂CO₃): δ=1.54 (b, 4H); 2.40 (b, 4H); 3.14 (t, 2H); 3.70 (s, 3H); 3.77 (s, 3H); 4.30 (d, 2H); 6.39 (d, 1H); 6.48-6.57 (m, 2H); 6.68 (d, 1H); 7.00 (t, 1H); 7.10 (d, 1H); 7.42 (t, 1H) ppm.

### Intermediate INT75

### 6-(1,1-difluoro-2-pyrrolidin-1-yl-ethyl)-pyridin-2-ylamine

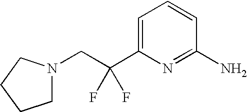

1.34 g of the compound described under INT74 is dissolved in 70 ml dichlormethane, mixed with 14 ml trifluoroacetic acid and stirred for 1 hour at ambient temperature. 50 ml sodiumhydrogencarbonate solution is added and extracted three times with 50 ml dichloromethane. The united organic phases are dried over sodium sulfate. 520 mg of title compound as raw product is yielded and used without purification.

¹H NMR (DMSO-d6, stored over K₂CO₃): δ=1.58 (b, 4H); 2.49 (b, 4H); 3.14 (t, 3H); 6.15 (s, 2H); 6.46 (d, 1H); 6.69 (d, 1H); 7.42 (t, 1H) ppm.

### Intermediate INT76

### 4-[2-(3-nitro-phenoxy)-acetyl]-piperazine-1-carboxylic acid tert-butylester

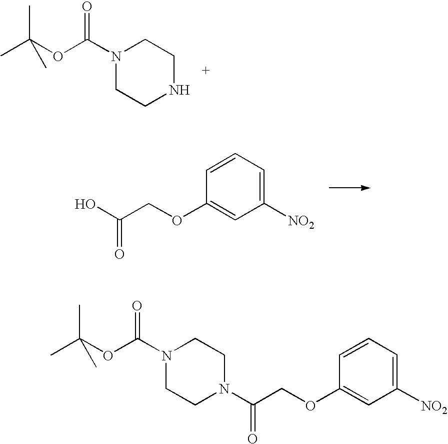

3-nitrophenoxyacetic acid (9.3 g, 50 mmol) is dissolved in dimethylacetamide (200 ml) and dripped in at ambient temperature among argon SOCl₂ (7.4 ml, 102 mmol) within 5 minutes. It is stirred for 30 minutes at ambient temperature and then the boc-piperazine (19.1 g, 102 mmol) is added in portions subject to ice cooling. It was stirred for 50 minutes at ambient temperature among argon and then the reaction mixture was poured onto water (500 ml), neutralized with sodiumcarbonate and extracted with ethyl acetate (3×100 ml). The united organic phases were washed with water (3×100 ml), dried over sodium sulfate and the solvent was distilled off in the vacuum. The title compound is obtained in a quantitative yield as black oil, which slowly completely crystallizes. The raw product was used in the next step without further repurification.

¹H-nMR (CDCl₃): δ=1.49 (s, 9H); 3.42 (m, 4H); 3.50 (m, 4H); 4.82 (s, 2H); 7.32 (dd, 1H); 7.48 (t, 1H); 7.77 (m, 1H); 0.88 (dd, 1H) ppm.

### Intermediate INT77

### 4-[2-(3-amino-phenoxy)-acetyl]-piperazine-1-carboxylic acid tert-butyl ester

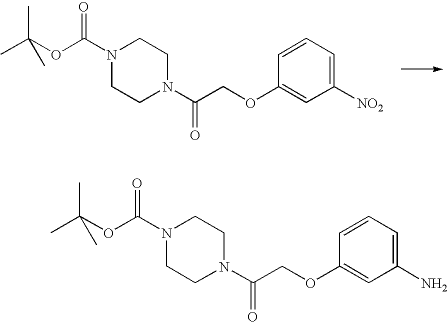

22 g (50 mmol) of the compound described under INT76 is dissolved in methanol (600 ml). Pd/C (4 g) is added among argon and hydrated until the hydrogen incorporation is complete. The catalyst is filtered off and the solvent distilled off in the vacuum. The title compound is obtained in the form of a viscous brown oil in a quantitative yield. The raw product is used in the next step without any further purification.

¹H-nMR (CDCl₃): δ=1.48 (s, 9H); 3.41 (m, 4H); 3.59 (m, 4H); 4.68 (s, 2H); 6.31 (m, 3H); 7.07 (t, 1H) ppm.

### Intermediate INT78

### 3-(3-nitrophenyl)-propionaldehyde

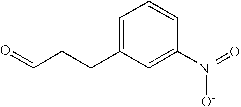

2.81 g Dess-martin periodinanes are added to a solution of 0.80 g 3-(3-nitrophenyl)-1-propanol (ref. J. Med. Chem., 1989, 32, 2104) in 100 ml dichlormethane. It is stirred for 2 hours at ambient temperature. 50 ml 10% sodiumthiosulfate solution and 50 ml saturated sodiumhydrogen carbonate solution is added, it is stirred for 10 minutes at ambient temperature and the dichloromethane is distilled off on the rotary evaporator. The residue is extracted twice with 100 ml acetic acid ethylester, the united organic phases are washed consecutively with 100 ml water and with 100 ml saturated table salt solution and dried over sodium sulfate. After condensing off the solvent on the rotary evaporator, 780 mg of title compound as raw product is yielded, which is further used without further purification.

¹H-nMR (CDCl₃): δ=2.86 (t, 2H); 3.06 (t, 2H); 7.44-7.49 (m, 1H); 7.55 (d, 1H); 8.08 (m, 2H); 9.83 (s, 1H) ppm.

### Intermediate INT79

### 1-[3-(3-nitro-phenyl)-propyl]-piperidine

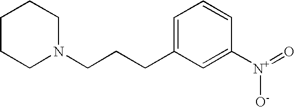

1.27 ml piperidine and 0.16 g sodium cyanoborohydride is added to a solution of 0.46 g of the compound produced under INT78 in 10 ml methanol. It is stirred for 3 hours at ambient temperature and 50 ml water and 40 ml acetic acid ethylester is added. The phases are separated and the aqueous phase is extracted twice with 40 ml acetic acid ethylester. The united organic phases are washed with 40 ml saturated table salt solution and dried over sodium sulfate. After purification by chromatography on silica gel, 635 mg of title compound is obtained.

¹H-nMR (CDCl₃): δ=1.41-1.48 (m, 2H); 1.57-1.65 (m, 4H); 1.87 (q, 2H); 2.32-2.44 (m, 6H); 2.75 (t, 2H); 7.43 (t, 1H); 7.52 (d, 1H); 8.06 (m, 2H) ppm.

### Intermediate INT80

### 6-fluoro-pyridin-2-ylamine

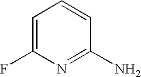

13 g 2,6-difluoropyridine and 15 ml of a 25% aqueous ammoniumhydroxide solution is stirred for 24 hours at 125° C. in a [sic: elbow pipe]. The reaction mixture is cooled to 0° C. and stirred for 2 hours at that temperature. The resulting solid substance is filtered off and dried at 40° C. in the vacuum. 5.0 g of title compound is obtained.

Mol. weight/MS (ESI) [M+1]⁺: 112.107/113. ¹H NMR (CDCl₃): δ=4.52, (bs, 2H), 6.24 (dd, 1H); 6.35 (dd, 1H); 7.50 (q, 1H) ppm.

### Intermediate INT81

### (6-fluoro-pyridin-2-yl)-carbamic acid tert-butyl ester

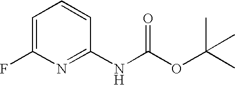

0.5 g of the compound described under INT80 is dissolved in 10 ml tetrahydrofurane, mixed with 10 mg dimethylaminopyridine, 1.57 ml diisopropylethylamine and 0.97 g di-tert-butyldicarbonate and then stirred for 4 hours at ambient temperature. 100 ml acetic acid ethylester is added and it is washed with water (50 ml). The organic phase is dried over sodium sulfate. After purification by chromatography on silica gel, 100 mg of title compound is obtained.

Mol. weight/MS (ESI) [M+1]⁺: 212.226/213. ¹H NMR (CDCl₃): δ=1.54, (s, 9H), 6.56 (dd, 1H); 7.08 (bs, 1H); 7.74 (m, 2H) ppm.

### Intermediate INT82

### [6-(2-methoxy-ethylamino)-pyridin-2-yl]-carbamic acid tert-butyl ester

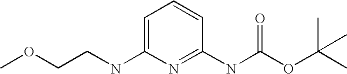

1.0 g of the compound described under INT81 and 5.0 ml 2-methoxy-ethylamine is stirred for 48 hours at 80° C. The reaction mixture is vacuum-condensed. The residue is incorporated with 100 ml acetic acid ethylester, and washed consecutively with 50 ml water and with 50 ml saturated table salt solution and dried over sodium sulfate. After purification by chromatography on silica gel, 500 mg of title compound is obtained.

Mol. weight/MS (ESI) [M+1]⁺: 267.331/268. ¹H NMR (CDCl₃): 1.50, (s, 9H); 3.32 (s, 3H); 3.42 (m, 2H); 3.52 (m, 2H); 4.64 (t, 1H); 6.08 (d, 1H); 6.90 (s, 1H); 7.18 (d, 1H); 7.34 (t, 1H) ppm.

### Intermediate INT83

### N-(2-methoxy-ethyl)-pyridine-2,6-diamine

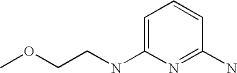

0.51 g of the compound described under INT82 is dissolved in 5 ml dichloromethane and mixed with 4.0 ml of 4 molar HCl in dioxane. It is stirred for 48 hours at ambient temperature. The solvent is distilled off in the vacuum, the residue is incorporated with 100 ml acetic acid ethylester and washed consecutively with 50 ml of 1 normal sodiumhydrogen carbonate solution, 50 ml water and 50 ml saturated table salt solution and dried over sodium sulfate. After the solvent is distilled off, 300 mg of title compound is obtained.

Mol. weight/MS (ESI) [M+1]⁺: 167.212/168. ¹H NMR (DMSO-d6, supported via K₂CO₃): δ=3.36 (s, 3H); 3.42 (m, 2H); 3.54 (m, 2H); 4.16 (s, 2H); 4.60 (s, 1H); 5.80 (m, 2H); 7.18 (t, 1H) ppm.

### Intermediate INT84

### 3,5,6-trifluoro-pyridin-2-ylamine

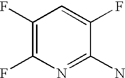

5.0 g 2,3,5,6-tetrafluoropyridine, 140 ml tetrahydrofurane and 25 ml of 25% aqueous ammonium hydroxide solution is stirred for 48 hours at 60° C. in an [sic: elbow pipe]. The reaction mixture is mixed with 100 ml water and extracted three times with 150 ml diethylether. After drying over sodium sulfate and distilling off the solvent, 3.5 g of title compound as raw product is yielded, which is used without further purification.

Mol. weight/MS (ESI) [M+1]⁺: 148.088/149.

The following compounds are produced according to the method described above.

| | | | Mol. weight/ | Educt/ |
|---|---|---|---|---|
| Intermediate | | | MS (ESI) | synthesis |
| no. | Structure and name | ¹H-nMR | [M + 1]⁺ | analogous |
| INT85 | 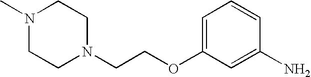 3-[2-(4-methyl-piperazin-1- yl)-ethoxy]-phenylamine | (DMSO-d6, stored over K₂CO₃): δ = 2.11 (s, 3H); 2.29 (m, 4H); 2.40 (m, 4H); 2.60 (m, 2H); 3.90 (m; 2H); 4.92 (s, 2H); 6.03 (dd, 1H); 6.10 (m, 2H); 6.82 (m, 1H) ppm. | 235.33/236 | INT86/INT77 |
| INT86 | 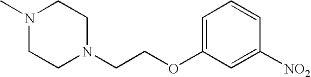 3-[2-(4-methyl-piperazin-1- yl)-ethoxy]-nitrobenzene | (CDCl₃): δ = 2.31 (s, 3H); 2.50 (m, 4H); 2.62 (m, 4H); 2.83 (m, 2H); 4.19 (m, 2H); 7.21 (m, 1H); 7.41 (t, 1H); 7.78 (m, 1H); 7.82 (dd, 1 H) ppm. | 265.31/266 | 3-nitro- phenol/ INT16 |
| INT87 | 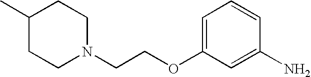 3-[2-(4-methyl-piperidin-1- yl)-ethoxy]-phenylamine | (CDCl₃): δ = 0.90 (m, 4H); 1.30 (m, 2H); 1.62 (m, 2H); 2.08 (m, 2H); 2.75 (m, 2H); 2.96 (m, 2H); 3.62 (m, 2H); 4.05 (m, 2H); 6.30 (m, 2H); 7.04 (t, 1H); 8.01 (s, 1H) ppm. | 234.34/235 | INT88/INT77 |
| INT88 | 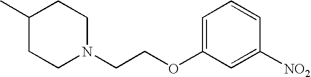 3-[2-(4-methyl-piperidin-1- yl)-ethoxy]-nitrobenzene | (CDCl₃): δ = 0.93 (d, 4H); 1.29 (m, 2H); 1.37 (m, 2H); 1.68 (m, 2H); 2.10 (m, 2H); 2.80 (m, 2H); 2.97 (d, 2H); 4.18 (m, 2H); 7.25 (m, 1H); 7.43 (t, 1H); 7.77 (m, 1H); 7.81 (dd, 1H) ppm. | 264.33/265 | 3-nitro- phenol/ INT16 |
| INT89 | 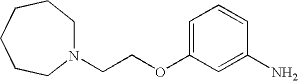 3-(2-azepan-1-yl-ethoxy)- phenylamine | (CDCl₃): δ = 1.68 (m, 8H); 2.81 (m, 4H); 2.99 (m, 2H); 3.65 (s, 2H); 4.08 (m, 2H); 6.29 (m, 3H); 7.04 (t, 1H) ppm. | 234.34/235 | INT90/INT77 |
| INT90 | 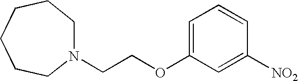 3-(2-azepan-1-yl-ethoxy)- nitrobenzene | (CDCl₃,): δ = 1.61 (m, 4H); 1.72 (m, 4H); 2.82 (m, 4H); 3.03 (m, 2H); 4.20 (m, 2H); 7.28 (m, 1H); 7.41 (m, 1H); 7.75 (d, 1H); 7.81 (d, 1H) ppm. | 264.33/265 | 3-nitro- phenol/ INT16 |
| INT91 | 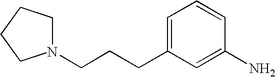 3-(3-pyrrolidin-1-yl-propyl)- phenylamine | (CDCl₃): δ = 1.74-1.86 (m, 6H); 2.42-2.68 (m, 8H); 3.54-3.70 (m, 2H); 6.49-6.54 (m, 2H); 6.60 (d, 1H); 7.07 (t, 1H); ppm. | | INT78/INT79 + INT77 |
| INT92 | 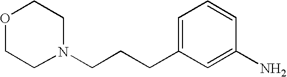 3-(3-morpholin-4-yl-propyl)- phenylamine | (CDCl₃): δ = 1.80 (q, 2H); 2.47 (t, 2H); 2.42-2.50 (m, 4H); 2.55 (t, 2H); 3.45-3.78 (m, 2H); 3.72 (q, 4H); 6.50-6.54 (m, 2H); 6.60 (d, 1H); 7.07 (t, 1H); ppm. | | INT78/INT79 + INT77 |
| INT93 | 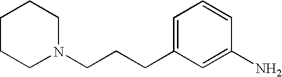 3-(3-piperidin-1-yl-propyl)- phenylamine | (CDCl₃): δ = 1.42 (m, 2H); 1.55-1.64 (m, 4H); 1.82 (q, 2H); 2.30-2.44 (m, 6H); 2.53 (t, 2H); 3.52-3.68 (m, 2H); 6.48-6.53 (m, 2H); 6.60 (d, 1H); 7.06 (t, 1H); ppm. | | INT79/INT77 |
| INT94 | 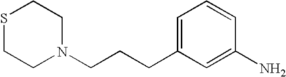 3-(3-thiomorpholin-4-yl- propyl)-phenylamine | (CDCl₃): δ = 1.75 (q, 2H); 2.33 (t, 6H); 2.47 (t, 2H); 2.60-2.71 (m, 8H); 3.34-3.68 (m, 2H); 6.43 (m, 2H); 6.51 (d, 1H); 7.00 (t, 1H); ppm. | | INT78/INT79 + INT77 |
| INT95 | 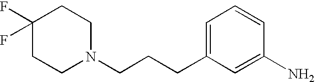 3-[3-(4,4-difluoro-piperidin- 1-yl)-propyl]-phenylamine | (DMSO-d6, stored over K₂CO₃): δ = 1.62 (q, 2H); 1.81-1.97 (m, 4H); 2.28 (t, 2H); 2.36-2.45 (m, 6H); 4.78-4.94 (s, 2H); 6.26-6.37 (m, 3H); 6.87 (t, 1H); ppm. | | INT78/INT79 + INT77 |
| INT96 | 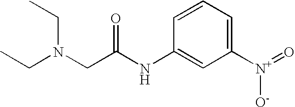 2-diethylamino-n-(3-nitro- phenyl)-acetamide | | 251.29/252 | INT 13/INT 14 |
| INT97 | 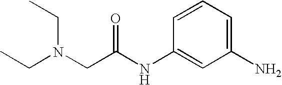 N-(3-amino-phenyl)-2- diethylamino-acetamide | | 221.30/222 | INT 17 |
| INT98 | 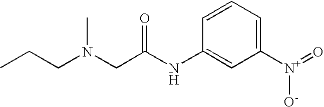 2-(methyl-propyl-amino)-n- (3-nitro-phenyl)-acetamide | | 251.29/252 | INT 13/INT 14 |
| INT99 | 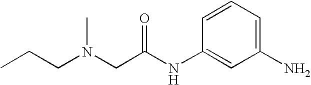 N-(3-amino-phenyl)-2- (methyl-propyl-amino)- acetamide | | 221.30/222 | INT 17 |
| INT100 | 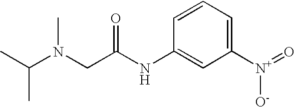 2-(isopropyl-methyl-amino)- n-(3-nitro-phenyl)-aetamide | | 251.29/252 | INT 13/INT 14 |
| INT101 | 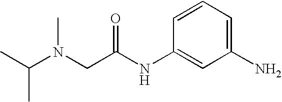 N-(3-amino-phenyl)-2-(iso- ropyl-methyl-amino)- acetamide | | 221.30/222 | INT 17 |
| INT102 | 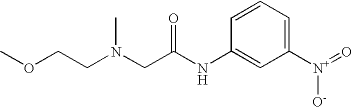 2-[(2-methoxy-ethyl)- methyl-amino]-n-(3-nitro- phenyl)-acetamide | | 267.29/268 | INT 13/INT 14 |
| INT103 | 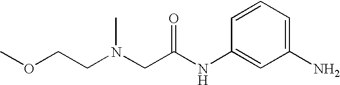 N-(3-amino-phenyl)-2-[(2- methoxy-ethyl)-methyl- amino]-acetamide | | 237.30/238 | INT 17 |
| INT104 | 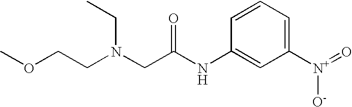 2-[ethyl-(2-methoxy-ethyl)- amino]-n-(3-nitro-phenyl)- acetamide | | 281.31/282 | INT 13/INT 14 |
| INT105 | 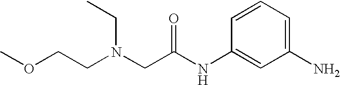 N-(3-amino-phenyl)-2- [ethyl-(2-methoxy-ethyl)- amino]-acetamide | | 251.33/252 | INT 17 |
| INT106 | 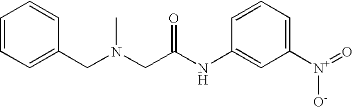 2-(benzyl-methyl-amino)-n- (3-nitro-phenyl)-acetamide | | 299.33/300 | INT 13/INT 14 |
| INT107 | 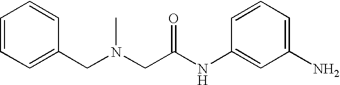 N-(3-amino-phenyl)-2- (benzyl-methyl-amino)- acetamide | | 269.35/267 | INT 17 |
| INT108 | 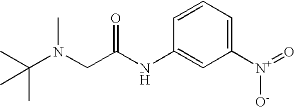 2-(tert-butyl-methyl-amino)- n-(3-nitro-phenyl)- acetamide | | 265.31/266 | INT 13/INT 14 |
| INT109 | 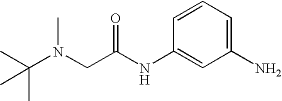 N-(3-amino-phenyl)-2-(tert- butyl-methyl-amino)- acetamide | | 235.33/236 | INT 17 |
| INT110 | 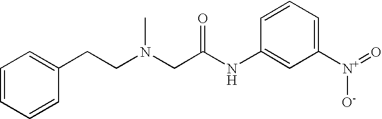 2-(methyl-phenethyl- amino)-n-(3-nitro-phenyl)- acetamide | | 313.36/314 | INT 13/INT 14 |
| INT111 | 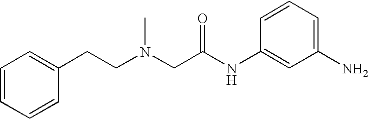 N-(3-amino-phenyl)-2- (methyl-phenethyl-amino)- acetamide | | 283.38/284 | INT 17 |
| INT112 | 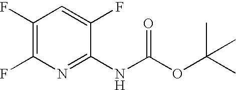 (3,5,6-trifluoro-pyridin-2-yl)- carbamic acid tert-butyl ester | | 248.207/249 | INT84/INT81 |
| INT113 | 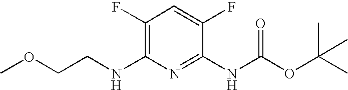 [3,5-difluoro-6-(2-methoxy- ethylamino)-pyridin-2-yl]- carbamic acid tert-butyl ester | | 303.311/304 | INT112/INT82 |
| INT114 | 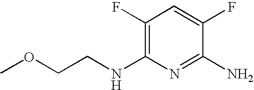 3,5-difluoro-n-(2-methoxy- ethyl)-pyridine-2,6-diamine | | 203.193/305 | INT113/INT83 |
| INT115 | 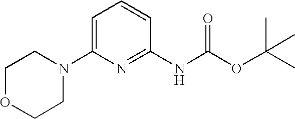 (6-morpholin-4-yl-pyridin-2- yl)-carbamic acid tert-butyl ester | (CDCl₃): δ = 1.50, (s, 9H); 3.30 (m, 4H); 3.66 (m, 4H); 6.28 (d, 1H); 6.86 (s, 1H); 7.20 (d, 1H); 7.48 (t, 1H) ppm. | 279.342/280 | INT81/INT82 |
| INT116 | 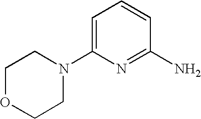 6-morpholin-4-yl-pyridin-2- ylamine | (CDCl₃): δ = 3.40 (m, 4H); 3.74 (m, 4H); 4.20 (s, 2H); 5.90 (d, 1H); 6.00 (d, 1H); 7.22 (t, 1H) ppm. | 179.223/180 | INT115/INT83 |
| INT117 | 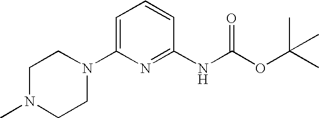 [6-(4-methyl-piperazin-1-yl)- pyridin-2-yl]-carbamic acid tert-butyl ester | (CDCl₃): δ = 1.56, (s, 9H); 2.58 (s, 3H); 2.50 (m, 4H); 3.50 (m, 4H); 6.26 (d, 1H); 6.90 (s, 1H); 7.16 (d, 1H); 7.48 (t, 1H) ppm. | 292.384/293 | INT81/INT82 |
| INT118 | 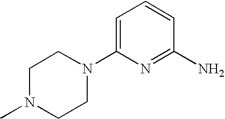 6-(4-methyl-piperazin-1-yl)- pyridin-2-ylamine | (CDCl₃): δ = 2.88 (s, 3H); 3.60 (m, 4H); 3.74 (m, 4H); 5.92 (d, 1H); 6.20 (d, 1H); 7.54 (t, 1H) ppm. | 192.266/193 | INT117/INT83 |
| INT119 | 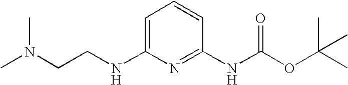 [6-(2-dimethylamino- ethylamino)-pyridin-2-yl]- carbamic acid tert-butyl ester | (CDCl₃): δ = 1.46, (s, 9H); 2.42 (s, 6H); 3.22 (m, 2H); 3.34 (m, 2H); 4.70 (t, 1H); 6.06 (d, 1H); 7.14 (d, 1H); 7.36 (t, 1H) ppm. | 280.373/ 281 | INT81/INT82 |
| INT120 | 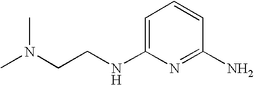 N-(2-dimethylamino-ethyl)- pyridine-2,6-diamine | (DMSO-d6): δ = 2.44 (s, 6H); 2.50 (t, 2H); 3.34 (m, 2H); 5.78 (m, 2H); 7.20 (t, 1H) ppm. | 180.255/181 | INT119/INT83 |
| INT121 | 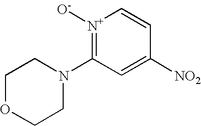 | (CDCl₃): δ = 3.46 (m, 4H), 3.94 (m, 4H); 7.66 (s, 1H); 7.72 (dd, 1H); 8.20 (d, 1H) ppm. | 225.206/226 | —/INT11 |
| INT122 | 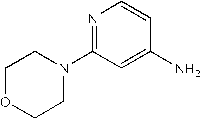 2-morpholin-4-yl-pyridin-4- ylamine | (CDCl₃): δ = 3.42 (m, 4H); 3.82 (m, 4H); 5.84 (s, 1H); 6.02 (d, 1H); 7.88 (d, 1H) ppm. | 179.223/180 | INT121/INT12 |
| INT123 | 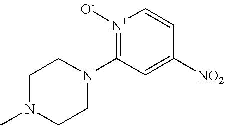 | (CDCl₃): δ = 2.36 (s, 3H); 3.40 (m, 4H); 3.74 (m, 4H); 7.22 (dd, 1H); 7.30 (s, 1H); 8.34 (d, 1H) ppm. | 238.248/239 | —/INT11 |
| INT124 | 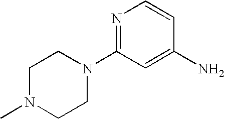 2-(4-methyl-piperazin-1-yl)- pyridin-4-ylamine | (CDCl₃): δ = 2.30 (s, 3H); 2.45 (m, 4H); 3.48 (m, 4H); 5.88 (s, 1H); 5.96 (dd, 1H); 7.84 (d, 1H) ppm. | 192.266/193 | INT123/INT12 |
| INT125 | 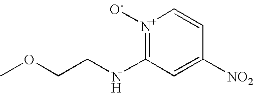 | (CDCl₃): δ = 3.42 (s, 3H); 3.64 (m, 2H); 3.72 (m, 2H), 7.14 (s, 1H); 7.46 (dd, 1H); 7.58 (dd, 1H); 7.26 (d, 1H) ppm. | 213.195/214 | —/INT11 |
| INT126 | 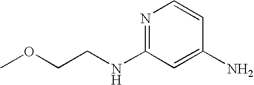 N*2*-(2-methoxy-ethyl)- pyridine-2,4-diamine | (DMSO-d6): δ = 3.40 (s, 3H); 3.46 (m, 2H); 3.64 (m, 2H); 5.54 (s, 1H); 5.60 (s, 2H); 5.80 (dd, 1H); 5.88 (t, 1H); 7.44 (d, 1H) ppm. | 167.212/168 | INT125/INT12 |

### 2. Template Synthesis

### Intermediate INTT1)

### cyano-ethylthiocarbamoyl-acetic acid ethylester

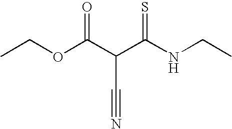

4.25 ml ethylisothiocyanate is added to a mixture of 5 g cyanoacetic acid ethylester and 5 ml triethylamine 25° C. Then it is left to stir for 6 hours at 50° C. After that, the reaction mixture is vacuum-condensed. The residue is incorporated into ethanol and poured onto 150 ml of ice cold 1 normal hydrochloric acid. It is left to stir for 3 hours at 25° C. and then the residue is filtered off. The solid substance yielded is washed with water. 7 g of product is yielded.

Mol mass=200.261; MS (ESI): [M+1]+=201.

### Intermediate INTT2)

### (E or Z)-cyano-(3-ethyl-4-oxo-thiazolidin-2-yliden)-acetic acid ethylester

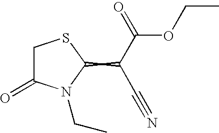

7.82 g of the compound described under INTT1) is dissolved in 100 ml tetrahydrofurane. A solution of 3.9 ml bromoacetyl chloride is added and left to stir for 8 hours at 25° C. The reaction mixture is then poured onto saturated aqueous sodium hydrogencarbonate. It is left to stir for 1 hour and then extracted with ethylacetate. The organic phase is washed with saturated sodium chloride solution, dried over sodium sulfate and vacuum-condensed. The raw product yielded is recrystallized from a mixture of ethylacetate/diisopropylester. 7.7 g of product is obtained.

1H-nMR (CDCl3): δ=1.36 (6H); 3.70 (2H); 4.32 (4H) ppm.

### Intermediate INTT3)

### (E or Z)-cyano-(5-(E/Z)-ethoxymethylen-3-ethyl-4-oxo-thiazolidin-2-yliden)-acetic acid ethylester

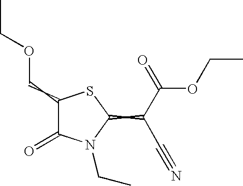

A mixture of 1.54 g of the substance described under INTT2), 2.5 ml triethylorthoformate and 3.5 ml acetic acid anhydride is boiled for 8 hours under reflux. The reaction mixture is then poured onto ice water. It is left to stir for 3 hours and then the residue is filtered off. The solid substance yielded is washed with water. 1.28 g of product is obtained.

1H-nMR (CDCl3): δ=1.38 (9H); 4.20-4.40 (6H); 7.72 (1H) ppm.

### Intermediate INTT4)

### (E or Z)-cyano-(3-ethyl-4-oxo-thiazolidin-2-yliden)-acetic acid allylester

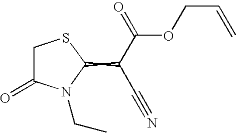

A solution of 37.6 ml cyanoacetic acid allylester in 60 ml dimethylformamide is added to a suspension of 12.8 g sodium hydride (60%) at 0° C. It is stirred for 10 minutes at 0° C. and then a solution of 28.0 ml ethylisothiocyanate in 60 ml dimethylformamide is added. It is then stirred for 2 hours at 25° C. A solution of 32 ml bromoacetylchloride in 60 ml dimethylformamide is then added at 0° C. and stirred for 15 hours at 25° C. The reaction mixture is then poured onto saturated sodiumhydrogencarbonate solution. Acetic acid ethylester is used to extract, the organic phase is washed with saturated sodiumchloride solution, dried over sodium sulfate and vacuum-condensed. The raw product is purified by column chromatography on silica gel with a mixture made from hexane/ethylacetate. 33.9 g of product is yielded.

1H-nMR (CDCl3): δ=1.23 (3H); 4.11 (2H); 4.71 (2H); 5.25 (1H); 5.37 (1H); 5.90-6.04 (1H) ppm.

### Intermediate INTT5)

### (E or Z)-cyano-(5-(E/Z)-ethoxymethylen-3-ethyl-4-oxo-thiazolidin-2-yliden)-acetic acid allylester

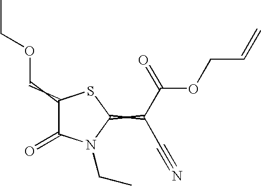

Analogous to intermediate INTT3), 14.8 g of product is yielded from 12.8 g of the compound described under INTT4), 20.9 ml triethylorthoformiate and 29.4 ml acetic acidanhydride.

1H-nMR (CDCl3): δ=1.32-1.45 (6H); 4.23 (2H); 4.38 (2H); 4.73 (2H); 5.29 (1H); 5.41 (1H), 5.92-6.05 (1H); 7.72 (1H) ppm.

### Intermediate INTT6)

### cyano-[3-ethyl-4-oxo-thiazolidin-(2-(E or Z))-ylidene]-acetic acid

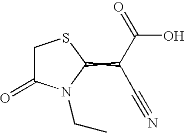

5.04 g of the compound described under INTT4) is dissolved in 300 ml tetrahydrofurane. 3.42 g of 1,3-dimethylbarbituric acid and 1.17 g Tetrakis-(triphenylphosphin)-palladium is added. It is stirred for 30 minutes at ambient temperature and the reaction mixture is condensed on the rotary evaporator until dry. The raw product yielded is used without any further purification.

1H-nMR (DMSO-d6, selected signals) δ=1.21 (t, 3H); 3.89 (s, 2H); 4.10 (q, 2H); 13.24 (s, b, 1H) ppm.

### Intermediate INTT7)

### 2-cyano-n-ethyl-2-[3-ethyl-4-oxo-thiazolidin-(2-(E or Z))-ylidene]-acetamide

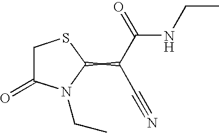

Approx. 4.15 g of the compound described under INTT6) (raw product that was yielded from 2.5 g of the compound described under INTT4) is dissolved in 100 ml dimethylformamide. 3.34 g sodiumhydrogencarbonate, 6.0 ml of a solution of ethylamine in tetrahydrofurane (c=2.0 M) and 3.88 g TBTU is added. It is stirred for 3 hours at ambient temperature. The reaction mixture is mixed with water and extracted with acetic acid ethylester. The organic solution is washed with saturated sodium chloride solution, dried over sodium sulfate and condensed. After purification by means of recrystallization from ethanol, 1.47 g of title compound is obtained.

1H-nMR (DMSO-d6): δ=1.05 (t, 3H); 1.21 (t, 3H); 3.18 (pentuplet, 2H); 3.70 (s, 2H); 4.10 (q, 2H); 7.81 (t, 1H) ppm.

The following compounds are produced according to the method described above.

| | | | | Educt/ |
|---|---|---|---|---|
| Example | | | Mol. | synthesis |
| no. | Structure and name | ¹H-nMR | weight | analogous |
| INTT8 | 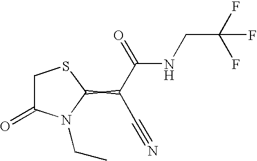 2-cyano-2-[3-ethyl-4-oxo- thiazolidin-(2-(E or Z))-ylidene]- n-(2,2,2-trifluoro-ethyl)- acetamide | (DMSO-d6, stored over K₂CO₃): δ = 1.22 (t, 3H); 3.84 (s, 2H); 3.95 (m, 2H); 4.11 (q, 2H); 8.33 (t, 1H) ppm. | 293.27 | INTT6/INTT7 |
| INTT9 | 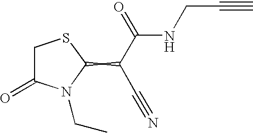 2-cyano-2-[3-ethyl-4-oxo- thiazolidin-(2-(E or Z))-ylidene]- n-prop-2-ynyl-acetamide | (DMSO-d6, stored over K₂CO₃): δ = 1.21 (t, 3H); 3.07 (m, 1H); 3.83 (s, 2H); 3.90 (m, 2H); 4.10 (q, 2H); 8.22 (t, 1H) ppm. | 249.29 | INTT6/INTT7 |
| INTT10 | 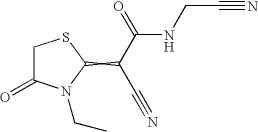 2-cyano-n-cyanomethyl-2-[3- ethyl-4-oxo-thiazolidin-(2-(E or Z))-ylidene]-acetamide | (DMSO-d6, stored over K₂CO₃): δ = 1.21 (t, 3H); 3.88 (s, 2H); 4.10 (q, 2H); 4.17 (d, 2H); 8.47 (t, 1H) ppm. | 250.28 | INTT6/INTT7 |
| INTT11 | 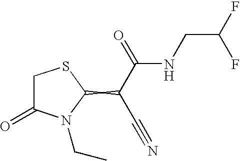 2-cyano-n-(2,2-difluoro-ethyl)-2- [3-ethyl-4-oxo-thiazolidin-(2-(E or Z))-ylidene]-acetamide | (DMSO-d6, stored over K₂CO₃): δ = 1.17 (t, 3H); 3.03 (m, 2H); 3.78 (s, 2H); 4.07 (q, 2H); 6.02 (m, 1H); 8.01 (m, 1H) ppm. | 275.27/276 | INTT6/INTT7 |
| INTT12 | 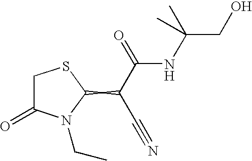 2-cyano-2-[3-ethyl-4-oxo- thiazolidin-(2-(E or Z))-ylidene]- n-(2-hydroxy-1,1-dimethyl-ethyl)- acetamide | (CDCl₃): δ = 1.32 (t, 3H); 1.36 (s, 6H); 3.60 (s, 2H); 3.68 (m, 2H); 4.24 (q, 2H); 6.30 (s, 1H) ppm. | 283.35/284 | INTT6/INTT7 |
| INTT13 | 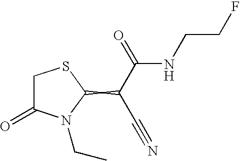 2-cyano-2-[3-ethyl-4-oxo- thiazolidin-(2-(E or Z))-ylidene]- n-(2-fluoro-ethyl)-acetamide | (CDCl₃): δ = 1.32 (t, 3H); 3.62 (s, 2H); 3.64 (m, 2H); 4.35 (q, 2H); 4.50 (m, 2H); 6.63 (s, 1H) ppm. | 257.28/258 | INTT6/INTT7 |

### 3. Synthesis of Ethylester- and Allylether Intermediates

### Intermediate INTE1

### Cyano-[3-ethyl-4-oxo-5-[1-[3-(2-pyrrolidin-1-yl-ethyl)-phenylamino]-meth-(E/Z)-ylidene]-thiazolidin-(2-(E or Z))-ylidene]-acetic acid ethyl ester

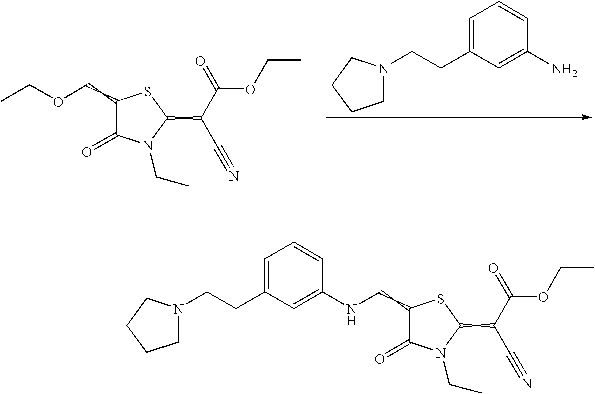

740 mg of the compound described under INT3) is dissolved in 50 ml Ethanol. 1.1 g of the compound described under INTT3) is added stirred for 5 hours under reflux. The solvent is condensed off on the rotary evaporator. After purification by chromatography on silica gel, 540 mg of title compound as pH-dependent 5-(E/Z)-isomer mixture is obtained.

1H-nMR (CDCl3, main isomer): δ=1.38 (t, 3H); 1.42 (t, 3H); 1.83 (m, 4H); 2.60 (m, 4H); 2.72 (m, 2H); 2.86 (m, 2H); 4.31 (q, 2H); 4.43 (q, 2H); 6.87-6.97 (m, 2H); 7.00 (d, 1H); 7.29 (t, 1H); 7.62 (d, 1H); 10.56 (d, 1H) ppm.

### Intermediate INTE2

### Cyano-[3-ethyl-4-oxo-5-[1-[3-(2-pyrrolidin-1-yl-ethyl)-phenylamino]-meth-(E/Z)-ylidene]-thiazolidin-(2-(E or Z))-ylidene]-acetic acid allyl ester

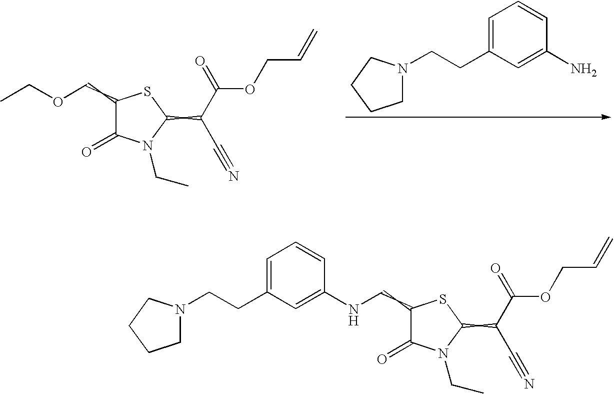

1.35 g of the compound described under INT3) is dissolved in 400 ml ethanol. 2.19 g of the compound described under INTT5) is added and stirred for 4 hours under reflux. The solvent is condensed off on the rotary evaporator. After purification by chromatography on silica gel, 2.2 g of title compound as pH-dependent 5-(E/Z)-isomer mixture is obtained.

1H-nMR (DMSO-d6, stored over K₂CO₃, main isomer): δ=1.24 (t, 3H); 1.69 (m, 4H); 2.50 (m, 4H); 2.66 (m, 2H); 2.76 (m, 2H); 4.25 (q, 2H); 4.71 (d, 2H); 5.26 (d, 1H); 5.38 (d, 1H); 5.90-6.08 (m, 1H); 6.96 (d, 1H); 7.12 (d, 1H); 7.22 (s, 1H); 7.26 (t, 1H); 8.22 (s, 1H); 10.53 (s, b, 1H) ppm.

### Intermediate INTE3

### Cyano-[3-ethyl-5-[1-[3-(2-hydroxy-2-methyl-propionylamino)-phenylamino]-meth-(E/Z)-ylidene]-4-oxo-thiazolidin-(2-(E or Z))-ylidene]-acetic acid allyl ester

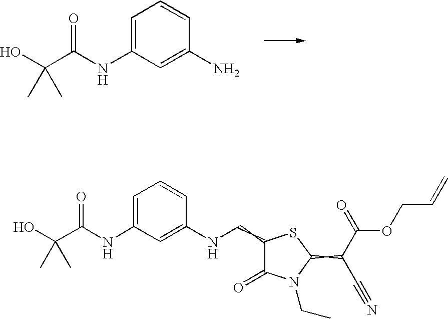

1.26 g of the compound described under INT6) is dissolved in 400 ml ethanol. 2.0 g of the compound described under INTT5) is added and stirred for 6 hours under reflux. After cooling, the reaction mixture is filtered and the solid substance yielded is recrystallized from ethanol. 1.4 g of title compound as pH-dependent 5-(E/Z)-isomer mixture is obtained. The solution obtained at filtration is condensed on the rotary evaporator. After purification by chromatography on silica gel, the residue yields another 1.1 g of title compound as pH-dependent 5-(E/Z)-isomer mixture.

1H-nMR (DMSO-d6, stored over K2CO3, main isomer): δ=1.28 (t, 3H); 1.38 (s, 6H); 4.26 (q, 2H); 4.72 (d, 2H); 5.27 (d, 1H); 5.39 (d, 1H); 5.76 (s, 1H); 5.90-6.08 (m, 1H); 6.99 (d, 1H); 7.27 (t, 1H); 7.46 (d, 1H); 7.89 (s, 1H); 8.16 (s, 1H); 9.67 (s, 1H); 10.63 (s, 1H) ppm.

### Intermediate INTE4

### Cyano-[3-ethyl-5-[1-(2-ethylamino-pyridin-4-ylamino)-meth-(E/Z)-ylidene]-4-oxo-thiazolidin-(2-(E or Z))-ylidene]-acetic acid allyl ester

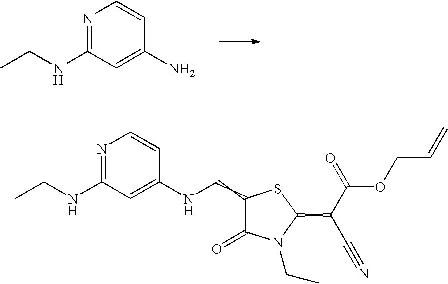

0.94 g of the compound described under INT12) is dissolved in 50 ml 1-propanol. 1.85 g of the compound described under INTT5) is added and it is stirred for 4 hours under reflux. After cooling, the reaction mixture is filtered. After purification by chromatography on silica gel, the solid substance yields 1.48 g of title compound as pH-dependent 5-(E/Z)-isomer mixture.

1H-nMR (DMSO-d6, stored over K2CO3, main isomer): δ=1.13 (t, 3H); 1.26 (t, 3H); 3.24 (pentuplet, 2H); 4.25 (q, 2H); 4.72 (d, 1H); 5.28 (d, 1H); 5.39 (d, 1H); 5.90-6.07 (m, 1H); 6.25 (d, 1H); 6.44 (dd, 1H); 6.49 (t, 1H); 7.85 (d, 1H); 8.13 (s, 1H); 10.47 (s, 1H) ppm.

### Intermediate INTE5

### Cyano-[5-[1-[6-(2,2-dimethyl-propionylamino)-pyridin-2-ylamino]-meth-(E/Z)-ylidene]-3-ethyl-4-oxo-thiazolidin-(2-(E or Z))-ylidene]-acetic acid allyl ester

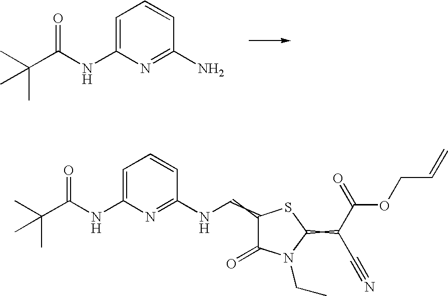

1.35 g of the compound described under INT9) is dissolved in 50 ml 1-propanol. 2.0 g of the compound described under INTT5) is added and it is stirred for 3 hours under reflux. After cooling, the reaction mixture is filtered and the solid substance yielded is recrystallized from ethanol. 2.47 g of title compound is yielded as pH-dependent 5-(E/Z)-isomer mixture.

1H-nMR (DMSO-d6, stored over K₂CO₃, main isomer): δ=1.20-1.31 (m, 12H); 4.27 (q, 2H); 4.72 (d, 2H); 5.28 (d, 2H); 5.39 (d, 2H); 5.91-6.06 (m, 1H); 6.29 (d, 2H); 7.68-7.80 (m, 2H); 8.86 (s, 1H); 9.71 (s, 1H); 10.94 (s, 1H) ppm.

The following compounds are produced according to the method described above.

| | | | Mol. weight/ | Educt/ |
|---|---|---|---|---|
| Example | | | MS (ESI) | synthesis |
| no. | Structure and name | ¹H-nMR | [M + 1]⁺ | analogous |
| INTE6 | 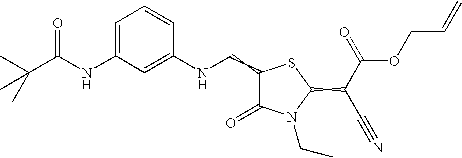 Cyano-[5-[1-[3-(2,2-dimethyl- propionylamino)-phenylamino]- meth-(E/Z)-ylidene]-3-ethyl-4- oxo-thiazolidin-(2-(E or Z))- ylidene]-acetic acid allyl ester | (DMSO-d6, stored over K₂CO₃, main isomer): δ = 1.19-1.32 (m, 12H); 4.27 (q, 2H); 4.72 (d, 2H); 5.27 (m, 1H); 5.39 (m, 1H); 5.91-6.07 (m, 1H); 6.99 (d, 1H); 7.28 (t, 1H); 7.39 (d, 1H); 7.78 (s, 1H); 8.13 (d, 1H); 9.28 (s, 1H); 10.67 (d, 1H) ppm. | 454.55/455 | INTT5/INTE2 |
| INTE7 | 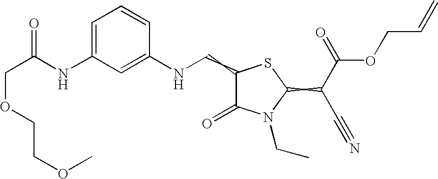 Cyano-[3-ethyl-5-[1-{3-[2-(2- methoxy-ethoxy)-acetylamino]- phenylamino}-meth-(E/Z)- ylidene]-4-oxo-thiazolidin-(2-(E or Z))-ylidene]-acetic acid allyl ester | (DMSO-d6, stored over K₂CO₃, main isomer): δ = 1.25 (t, 3H); 3.30 (s, 3H); 3.55 (m, 2H); 3.68 (m, 2H); 4.10 (s, 2H); 4.26 (q, 2H); 4.72 (d, 2H); 5.77 (d, 1H); 5.89 (d, 1H); 5.90-6.07 (m, 1H); 7.03 (m, 1H); 7.24-7.36 (m, 2H); 7.78 (s, 1 H); 8.15 (s, 1H); 9.72 (s, 1H); 10.69 (s, 1H) ppm. | 486.55/487 | INTT5/INTE2 |
| INTE8 | 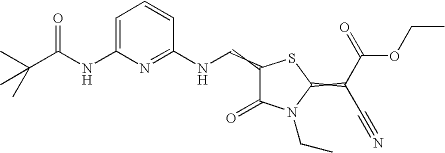 Cyano-[5-[1-[6-(2,2-dimethyl- propionylamino)-pyridin-2- ylamino]-meth-(E/Z)-ylidene]-3- ethyl-4-oxo-thiazolidin-(2-(E or Z))-ylidene]-acetic acid ethyl ester | (DMSO-d6, stored over K₂CO₃, main isomer): δ = 1.18-1.32 (m, 15H); 4.16-4.31 (m, 4H); 6.80 (d, 1H); 7.68-7.79 (m, 2H); 8.86 (s, 1H); 9.70 (s, 1H); 10.92 (s, 1H) ppm. | 443.53/444 | INTT3/INTE1 |
| INTE9 | 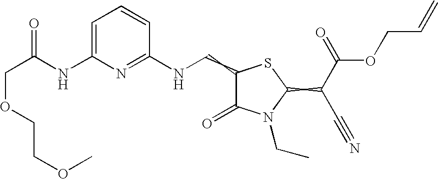 Cyano-[3-ethyl-5-[1-{6-[2-(2- methoxy-ethoxy)-acetylamino]- pyridin-2-ylamino}-meth-(E/Z)- ylidene]-4-oxo-thiazolidin-(2-(E or Z))-ylidene]-acetic acid allyl ester | (DMSO-d6, stored over K₂CO₃, main isomer): δ = 1.26 (t, 3H); 3.33 (s, 3H); 3.52 (m, 2H); 3.70 (m, 2H); 4.26 (q, 2H); 4.71 (d, 1H); 5.27 (d, 1H); 5.39 (d, 1H); 5.92-6.07 (m, 1H); 6.80 (d, 1H); 7.70-7.83 (m, 2H); 8.80 (s, 1H); 9.97 (s, 1H); 11.01 (s, 1H) ppm. | 487.53/488 | INTT5/INTE2 |
| INTE10 | 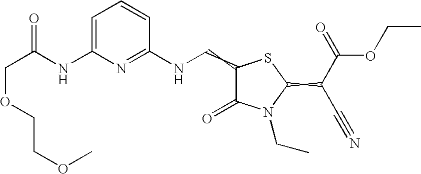 Cyano-[3-ethyl-5-[1-{6-[2-(2- methoxy-ethoxy)-acetylamino]- pyridin-2-ylamino}-meth-(E/Z)- ylidene]-4-oxo-thiazolidin-(2-(E or Z))-ylidene]-acetic acid ethyl ester | (DMSO-d6, stored over K₂CO₃, main isomer): δ = 1.20-1.32 (m, 6H); 3.32 (s, 3H); 3.53 (m, 2H); 3.70 (m, 2H); 4.25 (s, 2H); 4.20-4.31 (m, 4H); 6.82 (d, 1H); 7.71-8.84 (m, 2H); 8.74 (s, 1H); 10.00 (s, 1H); 10.98 (s, 1H) ppm. | 475.52/476 | INTT3/INTE1 |
| INTE11 | 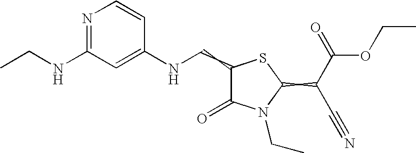 Cyano-[3-ethyl-5-[1-(2- ethylamino-pyridin-4-ylamino)- meth-(E/Z)-ylidene]-4-oxo- thiazolidin-(2-(E or Z))-ylidene]- acetic acid ethyl ester | (DMSO-d6, stored over K₂CO₃, main isomer): δ = 1.12 (t, 3H); 1.19-1.32 (m, 6H); 3.23 (m, 2H); 4.15-4.31 (m, 4H); 6.25 (d, 1H); 6.44 (dd, 1H); 6.49 (t, 1H); 7.35 (d, 1H); 8.11 (s, 1H); 10.46 (s, 1H) ppm. | 387.46/388 | INTT3/INTE1 |
| INTE12 | 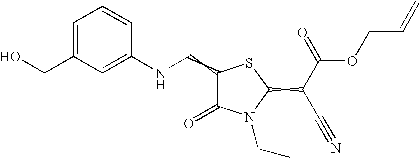 Cyano-[3-ethyl-5-[1-(3- hydroxymethyl-phenylamino)- meth-(E/Z)-ylidene]-4-oxo- thiazolidin-(2-(E or Z))-ylidene]- acetic acid allyl ester | (DMSO-d6, stored over K₂CO₃, main isomer): δ = 1.15 (t, 3H); 4.26 (q, 2H); 4.51 (d, 2H); 4.72 (d, 2H); 5.21-5.32 (m, 2H); 5.39 (d, 1H); 5.90-6.08 (m, 1H), 7.04 (d, 1H); 7.18 (d, 1H); 7.25-7.38 (m, 2H); 8.21 (s, 1H); 10.60 (s, 1H) ppm. | 385.44/386 | INTT5/INTE2 |
| INTE13 | 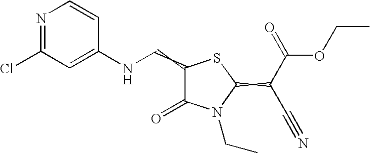 [5-[1-(2-chloro-pyridin-4- ylamino)-meth-(E/Z)-ylidene]-3- ethyl-4-oxo-thiazolidin-(2-(E or Z))-ylidene]-cyano-acetic acid ethyl ester | (DMSO-d6, stored over K₂CO₃, main isomer): δ = 1.19-1.32 (m, 6H); 4.19-4.31 (m, 4H); 7.30 (d, 1H); 7.44 (s, 1H); 8.21 (d, 1H); 8.32 (d, 1H); 10.67 (s, 1H) ppm. | 378.84/379 | INTT3/INTE1 |
| INTE14 | 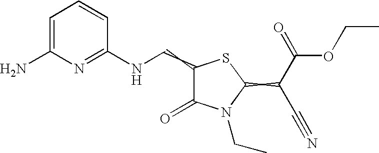 [5-[1-(6-amino-pyridin-2- ylamino)-meth-( E/Z)-ylidene]-3- ethyl-4-oxo-thiazolidin-(2-(E or Z))-ylidene]-cyano-acetic acid ethyl ester | (DMSO-d6, stored over K₂CO₃, main isomer): δ = 1.18-1.31 (m, 6H); 4.15-4.31 (m, 4H); 6.09-6.25 (m, 4H); 7.33 (t, 1H); 8.77 (s, 1H); 10.73 (s, 1H) ppm. | 359.41/360 | INTT3/INTE1 |
| INTE15 | 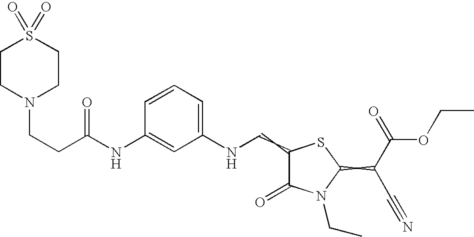 Cyano-[5-[1-{3-[3-(1,1-dioxo- 1lambda*6*-thiomorpholin-4-yl)- propionylamino]-phenylamino}- meth-(E/Z)-ylidene]-3-ethyl-4- oxo-thiazolidin-(2-(E or Z))- ylidene]-acetic acid ethyl ester | (DMSO-d6, stored over K₂CO₃, main isomer): δ = 1.16-1.33 (m, 6H); 2.48 (m, 2H); 2.86 (m, 2H); 2.93 (m, 4H); 3.08 (m, 4H); 4.13-4.30 (m, 4H); 7.00 (d, 1H); 7.19 (d, 1H); 7.28 (t, 1H); 7.73 (s, 1H); 8.10 (s, 1H); 10.40 (s, 1H); 10.64 (s, 1H) ppm. | 547.65/548 | INTT3/INTE1 |
| INTE16 | 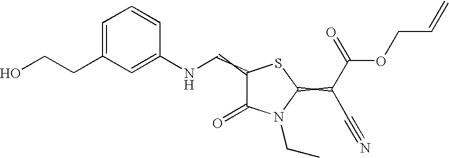 Cyano-[3-ethyl-5-[1-[3-(2- hydroxy-ethyl)-phenylamino]- meth-(E/Z)-ylidene]-4-oxo- thiazolidin-(2-(E or Z))-ylidene]- acetic acid allyl ester | (DMSO-d6, stored over K₂CO₃, main isomer): δ = 1.25 (t, 3H); 2.72 (t, 2H); 3.61 (q, 2H); 4.24 (q, 2H); 4.65 (t, 1H); 4.70 (d, 2H); 5.27 (d, 1 H); 5.39 (d, 1H); 5.91-6.08 (m, 1H); 6.94 (d, 1H); 7.11 (d, 1H); 7.18 (s, 1H); 7.24 (t, 1H); 8.23 (s, 1H); 10.55 (s, 1H) ppm. | 399.47/400 | INTT5/INTE2 |
| INTE17 | 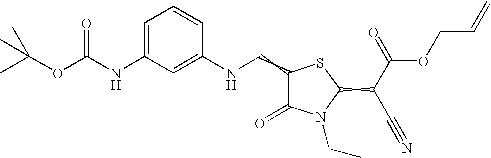 [5-[1-(3-tert- Butoxycarbonylamino- phenylamino)-meth-(E/Z)- ylidene]-3-ethyl-4-oxo-thiazolidin- (2-(E or Z))-ylidene]-cyano-acetic acid allyl ester | (DMSO-d6, stored over K₂CO₃, main isomer): δ = 1.07 (t, 3H); 1.26 (t, 3H); 4.25 (q, 2H); 4.71 (d, 2H); 5.28 (d, 1H); 5.39 (d, 1H); 5.91-6.08 (m, 1H), 6.92 (d, 1H); 7.09 (d, 1H); 7.21 (t, 1H); 7.60 (s, 1H); 8.11 (s, 1H); 9.48 (s, 1H); 10.67 (s, 1H) ppm. | 470.55/471 | INTT5/INTE2 |
| INTE18 | 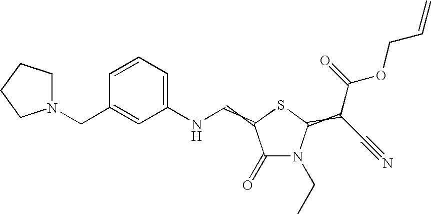 Cyano-[3-ethyl-4-oxo-5-[1-(3- pyrrolidin-1-ylmethyl- phenylamino)-meth-(E/Z)- ylidene]-thiazolidin-(2-(E or Z))- ylidene]-acetic acid allyl ester | ¹H-nMR (CDCl₃, 300 MHz) (selected peaks) δ = 1.38 (m, 3H); 1.75 (m, 4H); 2.48 (m, 4H); 3.59 (s, 2H); 4.41 (m, 2H); 4.72 (m, 2H); 5.23 (dd, 1H); 5.39 (dd, 1H); 5.97 (m, 1H); 6.97 (dd, 1H); 7.11 (m, 2H); 7.30 (m, 1H); 7.68 (s, 1H); 10.52 (s, 1H). | MW: 438.55 MS (ESI) [M + 1]⁺: 439 | INTT5/INTE2 |
| INTE19 | 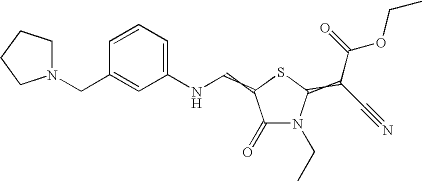 Cyano-[3-ethyl-4-oxo-5-[1-(3- pyrrolidin-1-ylmethyl- phenylamino)-meth-(E/Z)- ylidene]-thiazolidin-(2-(E or Z))- ylidene]-acetic acid ethyl ester | ¹H-nMR (CDCl₃, 300 MHz) (selected peaks) δ = 1.38 (m, 6H); 1.80 (m, 4H); 2.51 (m, 4H); 3.62 3H); 4.46 (m, 2H); 6.98 (dd, 1H); 7.10 (m, 2H); 7.30 (m, 1H); 7.68 (d, 1H); 10.58 (d, 1H). | MW: 426.54 MS (ESI) [M + 1]⁺: 427 | INTT3/INTE1 |
| INTE20 | 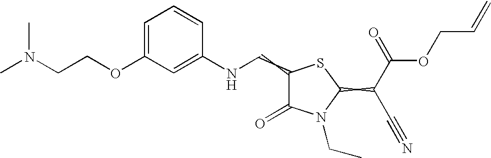 Cyano-[5-[1-[3-(2-dimethylamino- ethoxy)-phenylamino]-meth- (E/Z)-ylidene]-3-ethyl-4-oxo- thiazolidin-(2-(E or Z))-ylidene]- acetic acid allyl ester | ¹H-nMR (DMSO-d6, 300 MHz) (selected peaks) δ = 1.29 (m, 3H); 2.22 (s, 6H); 2.63 (m, 2H); 4.08 (m, 2H); 4.27 (m, 2H); 4.71 (d, 2H); 5.28 (dd, 1H); 5.39 (dd, 1H); 6.00 (m, 1H); 6.67 (dd, 1H); 6.91 (m, 1H); 7.24 (m, 1H); 8.22 (s, 1H); 10.48 (s, 1H). | MW: 442.54 MS (ESI) [M + 1]⁺: 443 | INTT5/INTE2 |
| INTE21 | 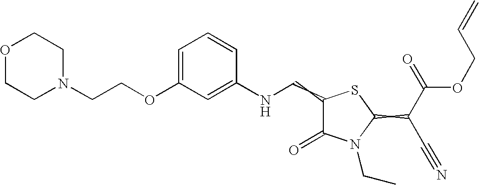 Cyano-[3-ethyl-5-[1-[3-(2- morpholin-4-yl-ethoxy)- phenylamino]-meth-(E/Z)- ylidene]-4-oxo-thiazolidin-(2-(E or Z))-ylidene]-acetic acid allyl ester | ¹H-nMR (CDCl₃, 300 MHz) (selected peaks) δ = 1.21 (m, 3H); 2.67 (m, 2H); 3.58 (m, 4H); 4.09 (m, 2H); 4.21 (m, 2H); 4.70 (d, 2H); 5.28 (dd, 1H); 5.40 (dd, 1H); 6.00 (m, 1H); 6.65 (dd, 1H); 6.86 (m, 2H); 7.21 (m, 1H); 8.16 (s, 1H); 10.39 (s, 1H). | MW: 484.57 MS (ESI) [M + 1]⁺: 485 | INTT5/INTE2 |
| INTE22 | 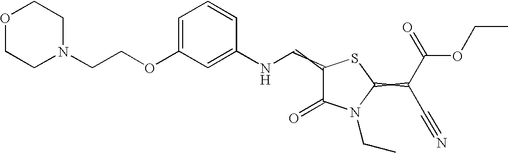 Cyano-[3-ethyl-5-[1-[3-(2- morpholin-4-yl-ethoxy)- phenylamino]-meth-(E/Z)- ylidene]-4-oxo-thiazolidin-(2-(E or Z))-ylidene]-acetic acid ethyl ester | ¹H-nMR (CDCl₃, 300 MHz) (selected peaks) δ = 1.36 (m, 6H); 2.59 (m, 4H); 2.81 (m, 2H); 3.73 (m, 4H); 4.11 (m, 2H); 4.30 (m, 2H); 4.42 (m, 2H); 6.65 (m, 3H); 7.23 (m, 1H); 7.58 (d, 1H); 10.50 (d, 1H). | MW: 472.56 MS (ESI) [M + 1]⁺: 473 | INTT3/INTE1 |
| INTE23 | 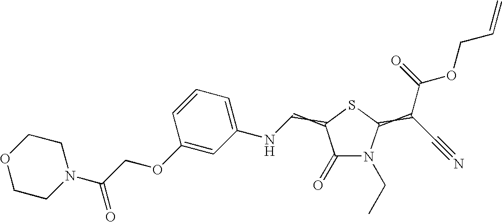 Cyano-[3-ethyl-5-[1-[3-(2- morpholin-4-yl-2-oxo-ethoxy)- phenylamino]-meth-(E/Z)- ylidene]-4-oxo-thiazolidin-(2-(E or Z))-ylidene]-acetic acid allyl ester | ¹H-nMR (DMSO- d6, 300 MHz) (selected peaks) δ = 1.29 (m, 3H); 3.36 (s, 2H); 3.62 (m, 4H); 4.28 (m, 2H); 4.71 (d, 2H); 4.87 (m, 2H); 5.29 (dd, 1H); 5.40 (dd, 1H); 6.01 (m, 1H); 6.68 (dd, 1H); 6.92 (m, 2H); 7.28 (m, 1H); 8.20 (d, 1H); 10.49 (d, 1H). | MW: 498.56 MS (ESI) [M + 1]⁺: 499 | INTT5/INTE2 |
| INTE24 | 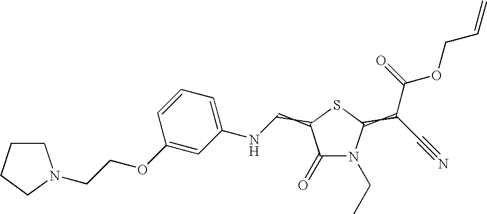 Cyano-[3-ethyl-4-oxo-5-[1-[3-(2- pyrrolidin-1-yl-ethoxy)- phenylamino]-meth-(E/Z)- ylidene]-thiazolidin-(2-(E or Z))- ylidene]-acetic acid allyl ester | ¹H-NMR (DMSO- d6, 300 MHz) (selected peaks) δ = 1.23 (m, 3H); 1.69 (m, 4H); 2.80 (m, 2H); 4.08 (m, 2H); 4.26 (m, 2H); 4.71 (d, 2H); 5.28 (dd, 1H); 5.40 (dd, 1H); 6.00 (m, 1H); 6.68 (dd, 1H); 6.90 (m, 2H); 7.27 (m, 1H); 8.27 (s, 1H); 10.48 (s, 1H). | MW: 468.58 MS (ESI) [M + 1]⁺: 469 | INTT5/INTE2 |
| INTE25 | 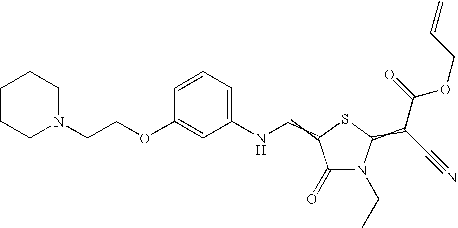 Cyano-[3-ethyl-4-oxo-5-[1-[3-(2- piperidin-1-yl-ethoxy)- phenylamino]-meth-(E/Z)- ylidene]-thiazolidin-(2-(E or Z))- ylidene]-acetic acid allyl ester | ¹H-NMR (DMSO- d6, 300 MHz) (selected peaks) δ = 1.25 (m, 3H); 1.39 (m, 2H); 1.49 (m, 4H); 2.63 (m, 2H); 4.10 (m, 2H); 4.25 (m, 2H); 4.72 (d, 2H); 5.28 (dd, 1H); 5.39 (dd, 1H); 5.98 (m, 1H); 6.65 (dd, 1H); 6.90 (m, 2H); 7.25 (m, 1H); 8.27 (s, 1H); 10.43 (s, 1H). | MW: 482.60 MS (ESI) [M + 1]⁺: 483 | INTT5/INTE2 |
| INTE26 | 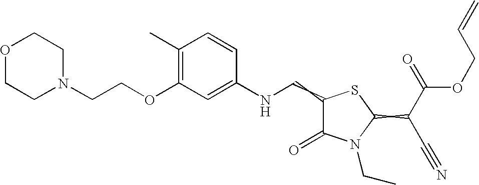 Cyano-[3-ethyl-5-[1-[4-methyl-3- (2-morpholin-4-yl-ethoxy)- phenylamino]-meth-(E/Z)- ylidene]-4-oxo-thiazolidin-(2-(E or Z))-ylidene]-acetic acid allyl ester | ¹H-NMR (CDCl₃, 300 MHz) (selected peaks) δ = 1.40 (m, 3H); 2.19 (s, 3H); 2.68 (m, 4H); 2.89 (m, 2H); 3.75 (m, 4H); 4.13 (m, 2H); 4.42 (m, 2H); 4.73 (m, 2H); 5.28 (dd, 1H); 5.41 (dd, 1H); 5.99 (m, 1H); 6.55 (m, 2H); 7.11 (m, 2H); 8.10 (d, 1H). | MW: 498.61 MS (ESI) [M + 1]⁺: 499 | INTT5/INTE2 |
| INTE27 | 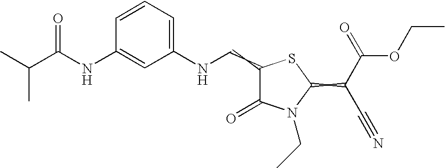 Cyano-{3-ethyl-5-[1-(3-isobutyryl- amino-phenylamino)-meth-(E/Z)- ylidene]-4-oxo-thiazolidin-(2-(E or Z))-ylidene}-acetic acid ethyl ester | (DMSO-d6, stored via K₂CO₃, primary isomer): δ = 1.10 (d, 6H); 1.21-1.28 (m, 6H); 2.58 (m, 1H); 4.19-4.25 (m, 4H); 6.93 (d, 1H); 7.17-7.25 (m, 2H); 7.72 (s, 1H); 8.06 (1H); 9.87 (s, 1H); 10.58 (1H) ppm. | 428.51/429 | INTT3/INTE1 |
| INTE28 | 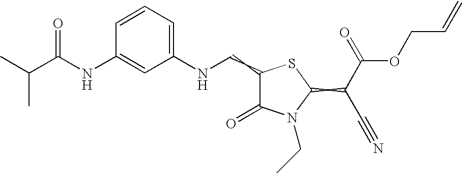 Cyano-{3-ethyl-5-[1-(3-isobutyryl- amino-phenylamino)-meth-(E/Z)- ylidene]-4-oxo-thiazolidin-(2-(E or Z))-ylidene}-acetic acid allyl ester | (DMSO-d6, stored via K₂CO₃, primary isomer): δ = 1.10 (d, 6H); 1.24 (t, 3H); 2.58 (m, 1H); 4.23 (q, 2H); 4.27 (d, 2H); 5.23-5.26 (m, 1H); 5.34-5.39 (m, 1H); 5.92-6.01 (m, 1H); 6.95 (d, 1H); 7.19-7.26 (m, 2H); 7.74 (s, 1H); 8.09 (1H), 9.88 (s, 1H); 10.62 (1H) ppm. | 440.53/441 | INTT5/INTE2 |
| INTE29 | 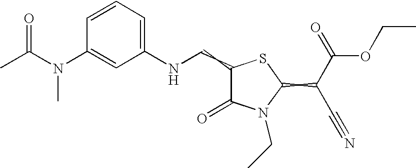 {5-[1-[3-(Acetyl-methyl-amino)- phenyl-amino]-meth-(E/Z)- ylidene]-3-ethyl-4-oxo-thiazolidin- (2-(E or Z))-ylidene}-cyanoacetic acid ethyl ester | (DMSO-d6, stored via K₂CO₃, primary isomer): δ = 1.21-1.27 (2t, 6H); 1.80 (3H); 3.15 (3H); 4.19-4.25 (m, 4H); 7.01 (d, 1H); 7.24-7.26 (m, 1H); 7.35-7.39 (m, 2H); 8.25 (d, 1H); 10.51 (1H) ppm. | 414.49/415 | INTT3/INTE1 |
| INTE30 | 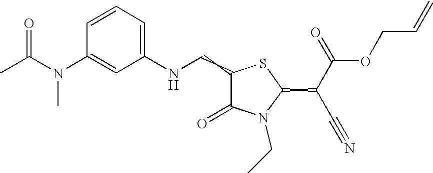 {5-[1-[3-(Acetyl-methyl-amino)- phenyl-amino]-meth-(E/Z)- ylidene]-3-ethyl-4-oxo-thiazolidin- (2-(E or Z))-ylidene}-cyano-acetic acid allyl ester | (DMSO-d6, stored via K₂CO₃, primary isomer): δ = 1.24 (t, 3H); 1.80 (3H); 3.15 (3H); 4.24 (q, 2H); 4.69-4.71 (m, 2H); 5.24-5.27 (m, 1H); 5.34-5.39 (m, 1H); 5.92-6.02 (m, 1H); 7.02 (d, 1H); 7.25-7.27 (m, 1H); 7.36-7.40 (m, 2H); 8.27 (1H); 10.51 (1H) ppm. | 426.50/427 | INTT5/INTE2 |
| INTE31 | 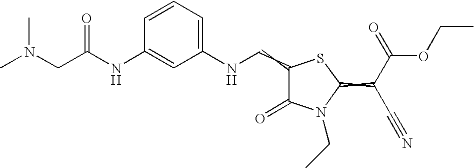 Cyano-{5-[1-[3-(2- dimethylamino-acetyl-amino)- phenylamino]-meth-(E/Z)- ylidene]-3-ethyl-4-oxo-thiazolidin- (2-(E or Z))-ylidene}-acetic acid ethyl ester | (DMSO-d6, stored via K₂CO₃, primary isomer): δ = 1.24 (t, 3H); 1.26 (t, 3H); 2.28 (s, 6H); 3.07 (s, 2H); 4.18-4.25 (m, 4H); 6.94-6.97 (m, 1H); 7.23 (t, 1H); 7.29-7.32 (m, 1H); 7.77 (1H); 8.09 (s, 1H); 9.76 (s, 1H); 10.58 (1H) ppm. | 443.53/444 | INTT3/INTE1 |
| INTE32 | 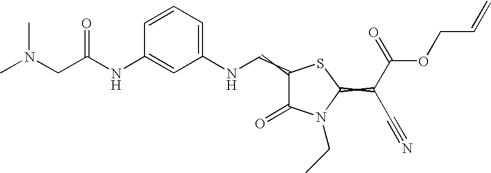 Cyano-{5-[1-[3-(2- dimethylamino-acetyl-amino)- phenylamino]-meth-(E/Z)- ylidene]-3-ethyl-4-oxo-thiazolidin- (2-(E or Z))-ylidene}-acetic acid allyl ester | (DMSO-d6, stored via K₂CO₃, primary isomer): δ = 1.24 (t, 3H); 2.28 (s, 6H); 3.08 (s, 2H); 4.24 (q, 2H); 4.69-4.71 (m, 2H); 5.23-5.27 (2q, 2H); 5.92-6.02 (m, 1H); 6.96-6.99 (m, 1H); 7.24 (t, 1H); 7.30-7.33 (m, 1H), 7.79 (1H); 8.12 (s, 1H); 9.78 (s, 1H); 10.62 (1H) ppm. | 455.54/456 | INTT5/INTE2 |
| INTE33 | 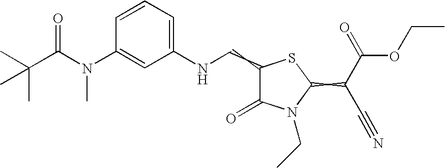 Cyano-[5-[1-{3-[(2,2-dimethyl- propionyl)-methyl-amino]- phenylamino}-meth-(E/Z)- ylidene]-3-ethyl-4-oxo-thiazolidin- (2-(E or Z))-ylidene]-acetic acid ethyl ester | (DMSO-d6, stored via K₂CO₃, primary isomer): δ = 1.00 (s, 9H); 1.24 (t, 3H); 1.26 (t, 3H); 3.10 (s, 3H); 4.19-4.26 (m, 4H); 6.99-7.01 (m, 1H); 7.28-7.39 (m, 3H); 8.23 (s, 1H); 10.49 (s, 1H) ppm. | 456.57/457 | INTT3/INTE1 |
| INTE34 | 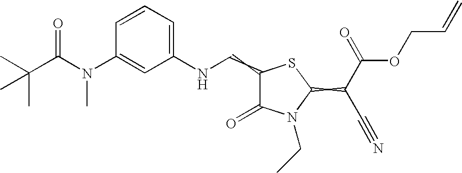 Cyano-[5-[1-{3-[(2,2-dimethyl- propionyl)-methyl-amino]- phenylamino}-meth-(E/Z)- ylidene]-3-ethyl-4-oxo-thiazolidin- (2-(E or Z))-ylidene]-acetic acid allyl ester | (DMSO-d6, stored via K₂CO₃, primary isomer): δ = 1.00 (s, 9H); 1.25 (t, 3H); 3.10 (s, 3H); 4.23 (q, 2H); 4.68-4.70 (m, 2H); 5.22-5.26 (m, 1H); 5.33-5.39 (m, 1H); 5.91-6.00 (m, 1H); 6.98-7.00 (m, 1H); 7.28-7.38 (m, 3H); 8.23 (1H); 10.49 (1H) ppm. | 468.58/467 | INTT5/INTE2 |
| INTE35 | 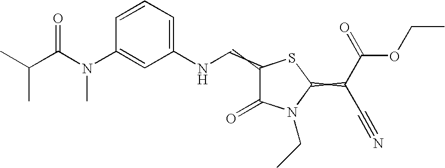 Cyano-{3-ethyl-5-[1-[3- (isobutyryl-methyl-amino)- phenylamino]-meth-(E/Z)- ylidene]-4-oxo-thiazolidin-(2-(E or Z))-ylidene}-acetic acid ethyl ester | (DMSO-d6, stored via K₂CO₃, primary isomer): δ = 0.93 (d, 6H); 1.22-1.28 (m, 6H); 2.47 (m, 1H); 3.14 (s, 3H); 4.20-4.26 (m, 4H); 7.00-7.02 (m, 1H); 7.29-7.42 (m, 3H); 8.25 (s, 1H); 10.51 (s, 1H) ppm. | 442.54/443 | INTT3/INTE1 |
| INTE36 | 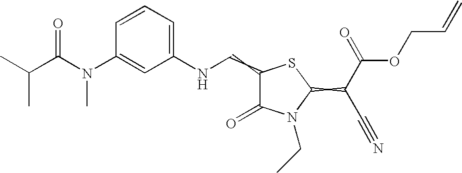 Cyano-{3-ethyl-5-[1-[3- (isobutyryl-methyl-amino)- phenylamino]-meth-(E/Z)- ylidene]-4-oxo-thiazolidin-(2-(E or Z))-ylidene}-acetic acid allyl ester | (DMSO-d6, stored via K₂CO₃, primary isomer): δ = 0.93 (d, 6H); 1.25 (t, 3H); 2.47 (m, 1H); 3.14 (s, 3H); 4.25 (q, 2H); 4.71 (d, 2H); 5.24-5.27 (m, 1H); 5.35-5.40 (m, 1H); 5.93-6.02 (m, 1H); 7.02 (d, 1H); 7.29-7.43 (m, 3H); 8.27 (s, 1H); 10.54 (s, 1H) ppm. | 454.55/455 | INTT5/INTE2 |
| INTE37 | 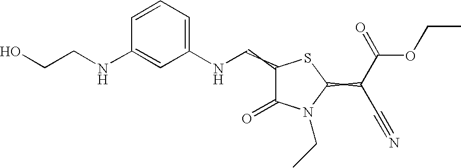 Cyano-{3-ethyl-5-[1-[3-(2- hydroxy-ethyl-amino)- phenylamino]-meth-(E/Z)- ylidene]-4-oxo-thiazolidin-(2-(E or Z))-ylidene}-acetic acid ethyl ester | (DMSO-d6, stored via K₂CO₃, primary isomer): δ = 1.21-1.28 (m, 6H); 3.09 (q, 2H); 3.54 (q, 2H); 4.19-4.26 (m, 4H); 4.70 (t, 1H); 5.75 (t, 1H); 6.32-6.35 (m, 1H); 6.43-6.49 (m, 2H); 7.02 (t, 1H); 8.10 (1H); 10.39 (H) ppm. | 402.48/403 | INTT3/INTE1 |
| INTE38 | 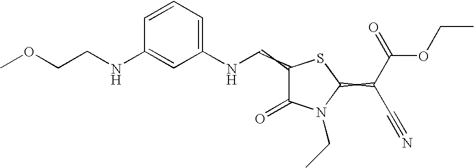 Cyano-{3-ethyl-5-[1-[3-(2- methoxy-ethylamino)- phenylamino]-meth-(E/Z)- ylidene]-4-oxo-thiazolidin-(2-(E or Z))-ylidene}-acetic acid ethyl ester | (DMSO-d6, stored via K₂CO₃, primary isomer): δ = 1.21-1.28 (m, 6H); 3.19 (q, 2H); 3.28 (s, 3H); 3.48 (t, 2H); 4.19-4.26 (m, 4H); 5.80 (t, 1H); 6.33-6.36 (dd, 1H); 6.44-6.49 (dd, 1H); 6.51 (m, 1H); 7.02 (t, 1H); 8.09-8.11 (m, 1H); 10.39 (1H) ppm. | 416.50/417 | INTT3/INTE2 |
| INTE39 | 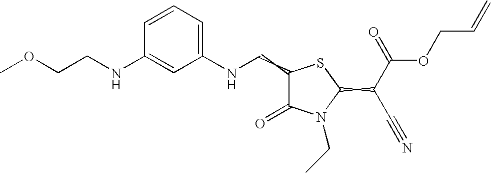 Cyano-{3-ethyl-5-[1-[3-(2- methoxy-ethylamino)- phenylamino]-meth-(E/Z)- ylidene]-4-oxo-thiazolidin-(2-(E or Z))-ylidene}-acetic acid allyl ester | (DMSO-d6, stored via K₂CO₃, primary isomer): δ = 1.24 (t, 3H); 3.19 (q, 2H); 3.28 (s, 3H); 3.47 (t, 2H); 4.23 (q, 2H); 4.68-4.70 (m, 2H); 5.23-5.26 (m, 1H); 5.34-5.39 (m, 1H); 5.80 (t, 1H); 5.92-6.01 (m, 1H); 6.32-6.35 (dd, 1H); 6.43-6.45 (dd, 1H); 6.49-6.51 (m, 1H); 7.01 (t, 1H); 8.10 (1H); 10.39 (1H) ppm. | 428.51/429 | INTT5/INTE2 |
| INTE40 | 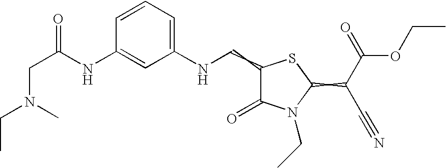 Cyano-[3-ethyl-5-[1-{3-[2-(ethyl- methyl-amino)-acetylamino]- phenylamino}-meth-(E/Z)- ylidene]-4-oxo-thiazolidin-(2-(E or Z))-ylidene]-acetic acid ethyl ester | (DMSO-d6, stored via K₂CO₃, primary isomer): δ = 1.04 (t, 3H); 1.22-1.28 (m, 6H); 2.28 (s, 3H); 3.12 (s, 3H); 4.20-4.26 (m, 4H); 6.97-6.99 (m, 1H); 7.24-7.34 (m, 2H); 7.78 (s, 1H); 8.13 (s, 1H); 8.73 (s, 1H); 10.61 (s, 1H) ppm. | 457.56/458 | INTT3/INTE1 |
| INTE41 | 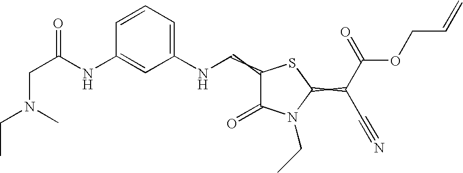 Cyano-[3-ethyl-5-[1-{3-[2-(ethyl- methyl-amino)-acetylamino]- phenylamino}-meth-(E/Z)- ylidene]-4-oxo-thiazolidin-(2-(E or Z))-ylidene]-acetic acid allyl ester | (DMSO-d6, stored via K₂CO₃, primary isomer): δ = 1.04 (t, 3H); 1.24 (t, 3H); 2.28 (s, 3H); 3.12 (s, 3H); 4.24 (q, 2H); 4.69-4.71 (m, 2H); 5.24-5.27 (m, 1H); 5.35-5.40 (m, 1H); 5.92-6.02 (m, 1H); 6.97-6.99 (m, 1H); 7.23-7.33 (m, 2H); 7.78 (1H); 8.13 (1H); 9.73 (s, 1H); 10.62 (s, 1H) ppm. | 469.57/470 | INTT5/INTE2 |
| INTE42 | 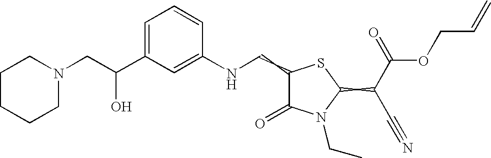 Cyano-[3-ethyl-5-[1-[3-(1- hydroxy-2-piperidin-1-yl-ethyl)- phenylamino]-meth-(E/Z)- ylidene]-4-oxo-thiazolidin-(2-(E or Z))-ylidene]-acetic acid allyl ester | (DMSO-d6, stored via K₂CO₃, primary isomer): δ = 1.20 (t, 3H); 1.26-1.54 (m, 6H); 2.22-2.44 (m, 6H); 4.21 (q, 2H); 4.58-4.74 (m, 3H); 4.92 (s, b, 1H); 5.22 (d, 1H); 5.33 (d, 1H); 5.84-6.04 (m, 1H); 6.91-7.11 (m, 2H); 7.13-7.33 (m, 2H); 8.20 (s, 1H); 10.56 (s, b, 1H) ppm | | INTT4 + INT54/5 |
| INTE43 | 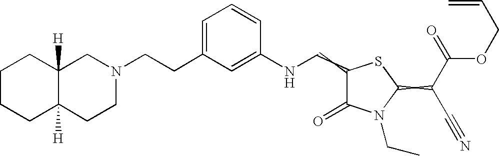 Cyano-[3-ethyl-5-[1-{3- [(4aR,8aS)-2-(octahydro- isoquinolin-2-yl)-ethyl]- phenylamino}-meth-(E/Z)- ylidene]-4-oxo-thiazolidin-(2-(E or Z))-ylidene]-acetic acid allyl ester | (DMSO-d6, stored via K₂CO₃, primary isomer): δ = 0.72-1.28 (m, 10H); 1.40-1.69 (m, 6H); 1.90 (t, 1H); 2.37-2.50 (m, 2H); 2.62-2.72 (m, 2H); 2.76 (d, 2H); 2.90 (d, 2H); 4.21 (q, 2H); 4.67 (d, 2H); 5.22 (d, 1H); 5.34 (d, 1H); 5.88-6.01 (m, 1H); 6.91 (d, 1H); 7.08 (d, 1H); 7.15-7.26 (m, 2H); 8.19 (s, 1H); 10.49 (s, b, 1H) ppm | | INTT5 + INT57/INTE2 |
| INTE44 | 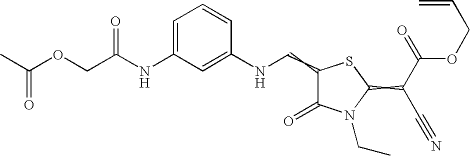 [5-[1-[3-(2-Acetoxy-acetylamino)- phenylamino]-meth-(E/Z)- ylidene]-3-ethyl-4-oxo-thiazolidin- (2-(E or Z))-ylidene]-cyano-acetic acid allyl ester | (DMSO-d6, stored via K₂CO₃, primary isomer): δ = 1.20 (t, 3H); 2.09 (s, 3H); 4.21 (q, 2H); 4.56-4.73 (m, 4H); 5.16-5.44 (dd, 1H); 5.83-6.06 (m, 1H); 6.98 (d, 1H), 7.16 (d, 1H); 7.25 (t, 1H); 7.64 (s, 1H); 8.09 (s, 1H), 10.11 (s, 1H); 10.63 (s, 1H) ppm | | INTT5 + INT27/INTE2 |
| INTE45 | 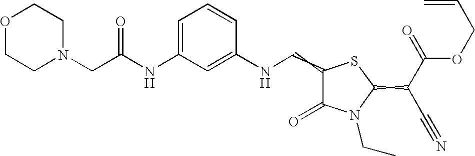 Cyano-[3-ethyl-5-[1-[3-(2- morpholin-4-yl-acetylamino)- phenylamino]-meth-(E/Z)- ylidene]-4-oxo-thiazolidin-(2-(E or Z))-ylidene]-acetic acid allyl ester | (DMSO-d6, stored via K₂CO₃, primary isomer): δ = 1.21 (t, 3H); 2.41-2.58 (m, 4H); 3.10 (s, 2H); 3.55-3.68 (m, 4H); 4.22 (q, 2H); 4.68 (d, 2H); 5.22 (dd, 1H), 5.35 (dd, 1H); 5.88-6.05 (m, 1H); 6.91-7.02 (m, 1H); 7.20-7.34 (m, 2H); 7.71 (s, 1H); 8.12 (s, 1H); 9.78 (s, 1H); 10.61 (s, b, 1H) ppm | | INTT5 + INT24/INTE2 |
| INTE46 | 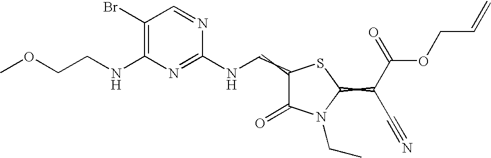 [5-[1-[5-Bromo-4-(2-methoxy- ethylamino)-pyrimidin-2- ylamino]-meth-(E/Z)-ylidene]-3- ethyl-4-oxo-thiazolidin-(2-(E or Z))-ylidene]-cyano-acetic acid allyl ester | (DMSO-d6, stored via K₂CO₃, primary isomer): δ = 1.20 (t, 3H); 3.25 (s, 3H); 3.42-3.65 (m, 4H); 4.20 (q, 2H); 4.69 (d, 2H); 5.22 (d, 1H); 5.34 (d, 1H); 5.87-6.03 (m, 1H); 7.29 (t, 1H); 8.13 (s, 1H); 8.57 (s, 1H); 11.18 (s, 1H) ppm | | INTT5 + INT64/INTE2 |
| INTE47 | 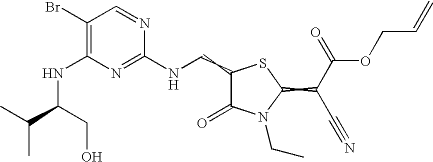 [5-[1-[5-Bromo-4-((R)-1- hydroxymethyl-2-methyl- propylamino)-pyrimidin-2- ylamino]-meth-(E/Z)-ylidene]-3- ethyl-4-oxo-thiazolidin-(2-(E or Z))-ylidene]-cyano-acetic acid allyl ester | (DMSO-d6, stored via K₂CO₃, primary isomer): δ = 0.84 (d, 3H); 0.90 (d, 3H); 1.20 (t, 3H); 1.89-2.01 (m, 1H); 3.49-3.63 (m, 2H); 4.01 (m, 1H); 4.20 (q, 2H); 4.68 (d, 2H); 4.72 (t, 1H); 5.22 (d, 1H); 5.34 (d, 1H); 5.88-6.01 (m, 1H); 6.40 (s, b, 1H); 8.11 (s, 1H); 8.69 (s, b, 1H); 11.13 (s, 1H) ppm | | INTT5 + INT67/INTE4 |
| INTE48 | 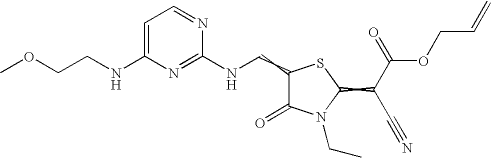 Cyano-[3-ethyl-5-[1-[4-(2- methoxy-ethylamino)-pyrimidin- 2-ylamino]-meth-(E/Z)-ylidene]-4- oxo-thiazolidin-(2-(E or Z))- ylidene]-acetic acid allyl ester | (DMSO-d6, stored via K₂CO₃, primary isomer): δ = 1.20 (t, 3H); 3.26 (s, 3H); 3.48 (b, 4H); 4.20 (q, 2H); 4.69 (d, 2H); 5.22 (d, 1H); 5.33 (d, 1H); 5.87-6.03 (m, 1H); 6.19 (d, 1H); 7.70 (s, b, 1H); 7.81 (s, b, 1H); 8.69 (s, 1H); 11.08 (s, b, 1H) ppm | | INTT5 + INT65/INTE4 |
| INTE49 | 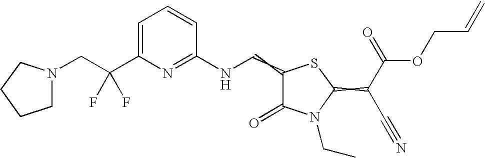 Cyano-[5-[1-[6-(1,1-difluoro-2- pyrrolidin-1-yl-ethyl)-pyridin-2- ylamino]-meth-(E/Z)-ylidene]-3- ethyl-4-oxo-thiazolidin-(2-(E or Z))-ylidene]-acetic acid allyl ester | (DMSO-d6, stored via K₂CO₃, primary isomer): δ = 1.22 (t, 3H); 1.57 (b, 4H); 2.50 (b, 4H); 3.18-3.36 (m, 2H); 4.21 (q, 2H); 4.69 (d, 2H); 5.23 (m, 1H); 5.35 (m, 1H); 5.84-6.06 (m, 1H); 7.16 (d, 1H); 7.32 (d, 1H); 7.87 (t, 1H); 8.67 (s, 1H); 11.11 (s, 1H) ppm | | INTT5 + INT75/INTE4 |
| INTE50 | 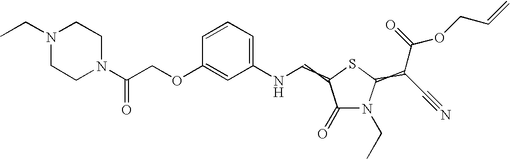 Cyano-[3-ethyl-5-[1-{3-[2-(4- ethyl-piperazin-1-yl)-2-oxo- ethoxy]-phenylamino}-meth- (E/Z)-ylidene]-4-oxo-thiazolidin- (2-(E or Z))-ylidene]-acetic acid allyl ester | (DMSO-d6, stored via K₂CO₃, primary isomer): δ = 0.99 (t, 3H); 1.20 (t, 3H); 2.32 (m, 6H); 3.41 (m, 4H); 4.23 (m, 2H); 4.69 (m, 2H); 4.82 (s, 2H); 5.21 (d, 1H); 5.32 (d, 1H), 5.97 (m, 1H); 6.62 (dd, 1H); 6.86 (s, 1H); 6.89 (d, 1H); 7.21 (t, 1H); 8.18 (s, 1H); 10.47 (s, 1H) ppm. | 525.63/526 | INTE76/INTE77 |
| INTE51 | 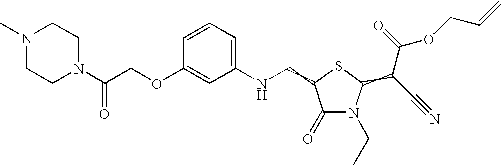 Cyano-[3-ethyl-5-[1-{3-[2-(4- methyl-piperazin-1-yl)-2-oxo- ethoxy]-phenylamino}-meth- (E/Z)-ylidene]-4-oxo-thiazolidin- (2-(E or Z))-ylidene]-acetic acid allyl ester | (DMSO-d6, stored via K₂CO₃, primary isomer): δ = 1.27 (m, 3H); 2.20 (s, 3H); 2.29 (m, 2H); 2.38 (m, 2H); 3.48 (m, 4H); 4.28 (m, 2H); 4.71 (m, 2H); 4.82 (s, 2H); 5.29 (d, 1H); 5.38 (d, 1H); 6.00 (m, 1H); 6.68 (dd, 1H); 6.89 (s, 1H); 6.92 (d, 1H); 7.28 (t, 1H); 8.20 (s, 1H); 10.51 (s, 1H) ppm. | 511.60/512 | INTE76/INTE77 |
| INTE52 | 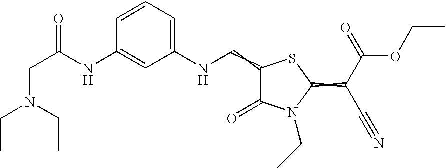 Cyano-[5-[1-[3-(2-diethylamino- acetylamino)-phenylamino]- meth-(E/Z)-ylidene]-3-ethyl-4- oxo-thiazolidin-(2-(E or Z))- ylidene]-acetic acid ethyl ester | (DMSO-d6, stored via K₂CO₃, primary isomer): δ = 1.02 (t, 6H); 1.24 (t, 3H); 1.26 (t, 3H); 2.61 (q, 4H); 3.16 (s, 2H); 4.22 (q, 2H); 6.99-7.01 (m, 1H); 7.24-7.34 (m, 2H); 7.77 (1H); 8.14 (1H); 9.71 (s, 1H); 10.60 (s, 1H) ppm. | 469.57/470 | INTT3/INTE1 |
| INTE53 | 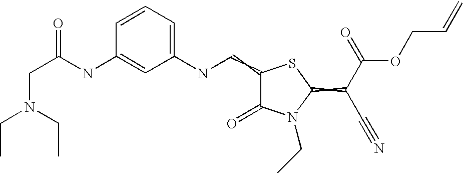 Cyano-[5-[1-[3-(2-diethylamino- acetylamino)-phenylamino]- meth-(E/Z)-ylidene]-3-ethyl-4- oxo-thiazolidin-(2-(E or Z))- ylidene]-acetic acid allyl ester | (DMSO-d6, stored via K₂CO₃, primary isomer): δ = 1.02 (t, 6H); 1.24 (t, 6H); 2.60 (q, 4H); 3.16 (s, 2H); 4.24 (q, 2H); 4.71 (m, 2H); 5.24-5.27 (m, 1H); 5.36-5.40 (m, 1H); 5.93-6.02 (m, 1H); 6.99-7.01 (m, 1H); 7.24-7.35 (m, 2H); 7.78 (1H); 8.16 (1H); 9.72 (s, 1H); 10.64 (s, 1H) ppm. | 483.59/484 | INTT5/INTE2 |
| INTE54 | 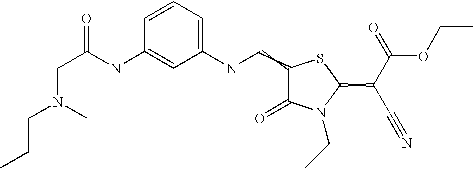 Cyano-[3-ethyl-5-[1-{3-[2- (methyl-propyl-amino)-acetyl- amino]-phenylamino}-meth- (E/Z)-ylidene]-4-oxo-thiazolidin- (2-(E or Z))-ylidene]-acetic acid ethyl ester | (DMSO-d6, stored via K₂CO₃, primary isomer): δ = 0.87 (t, 3H); 1.22-1.28 (m, 6H); 1.45-1.50 (m, 2H); 2.29 (s, 3H); 2.40 (t, 2H); 3.12 (s, 2H); 4.19-4.26 (m, 4H); 6.90-6.99 (m, 1H); 7.24-7.30 (m, 2H); 7.77 (1H); 8.12 (1H); 9.69 (s, 1H); 10.61 (s, 1H) ppm. | 471.58/472 | INTT3/INTE1 |
| INTE55 | 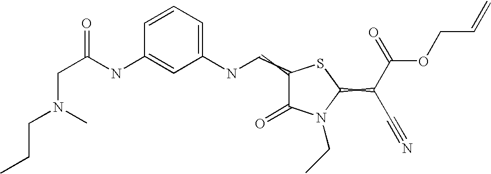 Cyano-[3-ethyl-5-[1-{3-[2- (methyl-propyl-amino)-acetyl- amino]-phenylamino}-meth- (E/Z)-ylidene]-4-oxo-thiazolidin- (2-(E orZ))-ylidene]-acetic acid allyl ester | (DMSO-d6, stored via K₂CO₃, primary isomer): δ = 0.88 (t, 3H); 1.24 (t, 3H); 1.43-1.52 (m, 2H); 2.29 (s, 3H); 2.40 (t, 2H); 3.13 (s, 2H); 4.24 (q, 2H); 4.71 (m, 2H); 5.24-5.27 (m, 1H); 5.36-5.40 (m, 1H); 5.93-6.02 (m, 1H); 6.99-7.01 (m, 1H); 7.24-7.31 (m, 2H); 7.78 (1H); 8.12-8.15 (1H); 9.72 (s, 1H); 10.63-10.66 (1H) ppm. | 483.59/484 | INTT5/INTE2 |
| INTE56 | 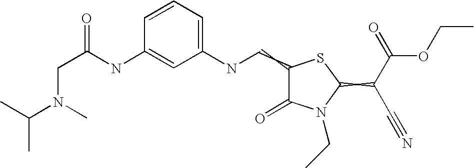 Cyano-[3-ethyl-5-[1-{3-[2- (isopropyl-methyl-amino)-acetyl- amino]-phenylamino}-meth- (E/Z)-ylidene]-4-oxo-thiazolidin- (2-(E or Z))-ylidene]-acetic acid ethyl ester | (DMSO-d6, stored via K₂CO₃, primary isomer): δ = 1.02 (d, 6H); 1.22-1.28 (m, 6H); 2.25 (s, 3H); 2.84-2.91 (m, 1H); 3.10 (s, 2H); 4.19-4.26 (m, 4H); 4.71 (m, 2H); 5.24-5.27 (m, 1H); 5.36-5.40 (m, 1H); 5.93-6.02 (m, 1H); 6.98-7.00 (m, 1H); 7.24-7.36 (m, 2H); 7.77 (1H); 8.14 (1H); 9.70 (s, 1H); 10.61 (s, 1H) ppm. | 471.58/472 | INTT3/INTE1 |
| INTE57 | 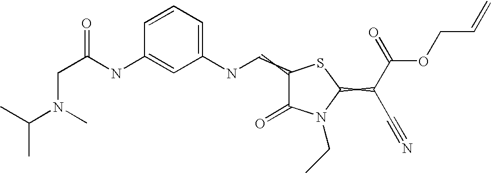 Cyano-[3-ethyl-5-[1-{3-[2- (isopropyl-methyl-amino)-acetyl- amino]-phenylamino}-meth- (E/Z)-ylidene]-4-oxo-thiazolidin- (2-(E or Z))-ylidene]-acetic acid allyl ester | (DMSO-d6, stored via K₂CO₃, primary isomer): δ = 1.02 (d, 6H); 1.24 (t, 3H); 2.25 (s, 3H); 2.84-2.91 (m, 1H); 3.10 (s, 2H); 4.23 (q, 2H); 4.71 (m, 2H); 5.24-5.27 (m, 1H); 5.36-5.40 (m, 1H); 5.93-6.02 (m, 1H); 6.98-7.00 (m, 1H); 7.24-7.36 (m, 2H); 7.77 (1H); 8.15 (1H); 9.69 (s, 1H); 10.63 (s, 1H) ppm. | 483.59/484 | INTT5/INTE2 |
| INTE58 | 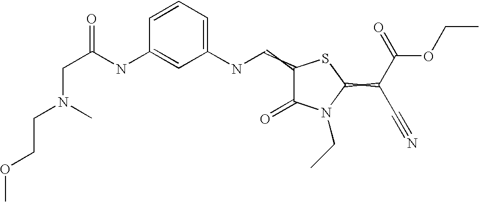 Cyano-[3-ethyl-5-[1-(3-{2-[(2- methoxy-ethyl)-methyl-amino]- acetylamino}-phenylamino)- meth-(E/Z)-ylidene]-4-oxo- thiazolidin-(2-(E or Z))-ylidene]- acetic acid ethyl ester | (DMSO-d6, stored via K₂CO₃, primary isomer): δ = 1.22-1.28 (m, 6H); 1.45-1.50 (m, 2H); 2.36 (s, 3H); 2.64 (t, 1H); 3.19 (s, 2H); 3.27 (s, 3H); 3.46 (t, 1H); 4.19-4.26 (m, 4H); 6.98-7.00 (m, 1H); 7.22-7.29 (m, 2H); 7.76 (1H); 8.12 (1H); 9.79 (s, 1H); 10.63 (s, 1H) ppm. | 487.58/488 | INTT3/INTE1 |
| INTE59 | 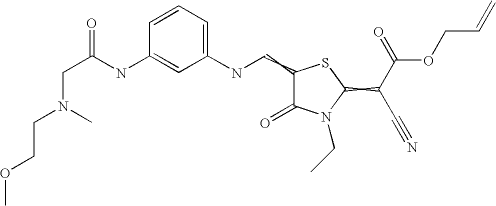 Cyano-[3-ethyl-5-[1-(3-{2-[(2- methoxy-ethyl)-methyl-amino]- acetylamino}-phenylamino)- meth-(E/Z)-ylidene]-4-oxo- thiazolidin-(2-(E or Z))-ylidene]- acetic acid allyl ester | (DMSO-d6, stored via K₂CO₃, primary isomer): δ = 1.24 (t, 3H); 1.45-1.50 (m, 2H); 2.36 (s, 3H); 2.64 (t, 1H); 3.18 (s, 2H); 3.27 (s, 3H); 3.46 (t, 1H); 4.24 (q, 2H); 4.71 (m, 2H); 5.24-5.27 (m, 1H); 5.36-5.40 (m, 1H); 5.93-6.03 (m, 1H); 6.99-7.01 (m, 1H); 7.22-7.30 (m, 2H); 7.77 (1H); 8.13 (1H); 9.79 (s, 1H); 10.65 (s, 1H) ppm. | 499.59/500 | INTT5/INTE2 |
| INTE60 | 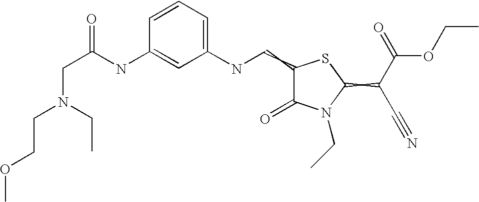 Cyano-[3-ethyl-5-[1-(3-{2-[ethyl- (2-methoxy-ethyl)-amino]-acetyl- amino}-phenylamino)-meth- (E/Z)-ylidene]-4-oxo-thiazolidin- (2-(E or Z))-ylidene]-acetic acid ethyl ester | (DMSO-d6, stored via K₂CO₃, primary isomer): δ = 1.01 (t, 3H); 1.24 (t, 3H); 1.26 (t, 3H); 2.36 (s, 3H); 2.66 (q, 2H); 2.72 (t, 2H); 3.21 (s, 2H); 3.26 (s, 3H); 3.44 (t, 1H); 4.20-4.26 (m, 4H); 7.00-7.02 (m, 1H); 7.21-7.30 (m, 2H); 7.74 (1H); 8.12-8.14 (1H); 9.79 (s, 1H); 10.62-10.64 (1H); ppm. | 501.61/502 | INTT3/INTE1 |
| INTE61 | 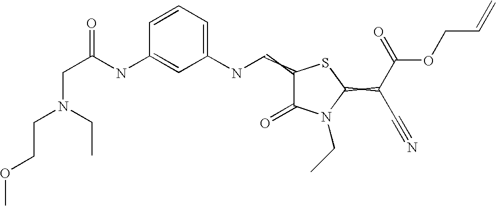 Cyano-[3-ethyl-5-[1-(3-{2-[ethyl- (2-methoxy-ethyl)-amino]-acetyl- amino}-phenylamino)-meth- (E/Z)-ylidene]-4-oxo-thiazolidin- (2-(E or Z))-ylidene]-acetic acid allyl ester | (DMSO-d6, stored via K₂CO₃, primary isomer): δ = 1.01 (t, 3H); 1.24 (t, 3H); 2.36 (s, 3H); 2.66 (q, 2H); 2.72 (t, 2H); 3.21 (s, 2H); 3.26 (s, 3H); 3.44 (t, 1H); 4.24 (q, 4H); 4.71 (m, 2H); 5.24-5.27 (m, 1H); 5.35-5.40 (m, 1H); 5.93-6.02 (m, 1H); 7.00-7.02 (m, 1H); 7.21-7.32 (m, 2H); 7.75 (1H); 8.13-8.15 (1H); 9.79 (s, 1H); 10.65-10.66 (1H) ppm. | 513.62/514 | INTT5/INTE2 |
| INTE62 | 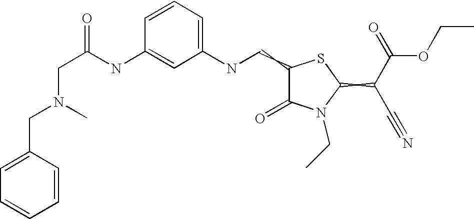 [5-[1-{3-[2-(Benzyl-methyl- amino)-acetylamino]-phenyl- amino}-meth-(E/Z)-ylidene]-3- ethyl-4-oxo-thiazolidin-(2-(E or Z)-ylidene]-cyano-acetic acid ethyl ester | (DMSO-d6, stored via K₂CO₃, primary isomer): δ = 1.22-1.28 (m, 6H); 1.26 (t, 3H); 2.27 (s, 3H); 3.19 (s, 2H); 3.65 (s, 2H); 4.19-4.26 (m, 4H); 6.98-7.00 (m, 1H); 7.21-7.40 (m, 7H); 7.78 (1H); 8.12 (1H); 9.81 (s, 1H); 10.63 (1H) ppm. | 519.63/520 | INTT3/INTE1 |
| INTE63 | 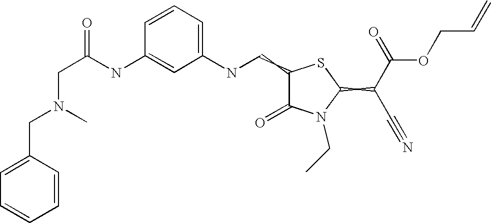 [5-[1-{3-[2-(Benzyl-methyl- amino)-acetylamino]-phenyl- amino}-meth-(E/Z)-ylidene]-3- ethyl-4-oxo-thiazolidin-(2-(E or Z))-ylidene]-cyano-acetic acid allyl ester | (DMSO-d6, stored via K₂CO₃, primary isomer): δ = 1.24 (t, 3H); 2.27 (s, 3H); 3.19 (s, 2H); 3.65 (s, 2H); 4.22-4.27 (q, 2H); 4.71 (m, 2H); 5.24-5.27 (m, 1H); 5.35-5.40 (m, 1H); 5.93-6.03 (m, 1H); 6.98-7.00 (m, 1H); 7.21-7.40 (m, 7H); 7.78 (1H); 8.14 (1H); 9.81 (s, 1H); 10.66 (1H) ppm. | 531.64/532 | INTT5/INTE2 |
| INTE64 | 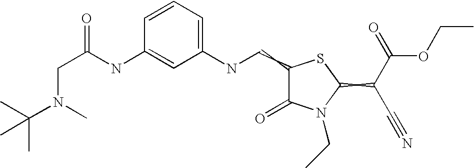 [5-[1-{3-[2-(tert-Butyl-methyl- amino)-acetylamino]-phenyl- amino}-meth-(E/Z)-ylidene]-3- ethyl-4-oxo-thiazolidin-(2 (E or Z)-ylidene]-cyano-acetic acid ethyl ester | (DMSO-d6, stored via K₂CO₃, primary isomer): δ = 1.09 (s, 9H); 1.22-1.28 (m, 6H); 2.25 (s, 3H) 3.11 (s, 2H); 4.19-4.26 (m, 4H); 7.00-7.02 (m, 1H); 7.25-7.37 (m, 2H); 7.74 (1H); 8.15 (1H); 9.68 (s, 1H); 10.61 (1H) ppm. | 485.61/486 | INTT3/INTE1 |
| INTE65 | 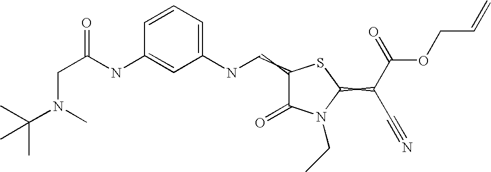 [5-[1-{3-[2-(tert-Butyl-methyl- amino)-acetylamino]-phenyl- amino}-meth-(E/Z)-ylidene]-3- ethyl-4-oxo-thiazolidin-(2-(E or Z))-ylidene]-cyano-acetic acid allyl ester | (DMSO-d6, stored via K₂CO₃, primary isomer): δ = 1.09 (s, 9H); 1.25, (t, 3H); 2.25 (s, 3H); 3.11 (s, 2H); 4.24 (q, 2H); 4.71 (m, 2H); 5.24-5.27 (m, 1H); 5.36-5.40 (m, 1H); 5.93-6.02 (m, 1H); 7.01-7.03 (m, 1H); 7.25-7.37 (m, 2H); 7.75 (1H); 8.16 (1H); 9.69 (s, 1H); 10.64 (1H) ppm. | 497.62/498 | INTT5/INTE2 |
| INTE66 | 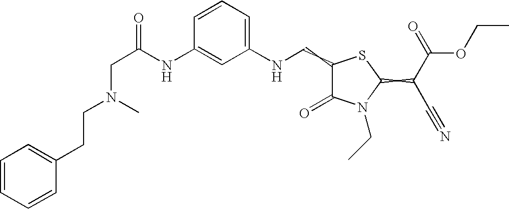 Cyano-[3-ethyl-5-[1-{3-[2- (methyl-phenethyl-amino)- acetylamino]-phenylamino}- meth-(E/Z)-ylidene]-4-oxo- thiazolidin-(2-(E or Z))-ylidene]- acetic acid ethyl ester | (DMSO-d6, stored via K₂CO₃, primary isomer): δ = 1.22-1.28 (m, 6H); 2.37 (s, 3H); 2.71-2.81 (m, 4H); 3.20 (s, 2H); 4.19-4.27 (m, 4H); 6.97-6.99 (m, 1H); 7.12-7.30 (m, 7H); 7.64 (1H), 8.11 (1H); 9.50 (s, 1H); 10.61 (1H) ppm. | 533.65/534 | INTT3/INTE1 |
| INTE67 | 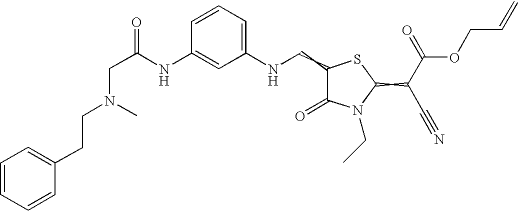 Cyano-[3-ethyl-5-[1-{3-[2- (methyl-phenethyl-amino)- acetylamino]-phenylamino}- meth-(E/Z)-ylidene]-4-oxo- thiazolidin-(2-(E or Z))-ylidene]- acetic acid allyl ester | (DMSO-d6, stored via K₂CO₃, primary isomer): δ = 1.24 (t, 3H); 2.37 (s, 3H); 2.71-2.81 (m, 4H); 3.20 (s, 2H); 4.25 (q, 4H); 4.70 (m, 2H); 5.24-5.27 (m, 1H); 5.35-5.40 (m, 1H); 5.93-6.02 (m, 1H); 6.97-6.99 (m, 1H); 7.12-7.30 (m, 2H); 7.64 (1H); 8.12 (1H); 9.50 (s, 1H); 10.64 (1H) ppm. | 545.67/546 | INTT5/INTE2 |
| INTE68 | 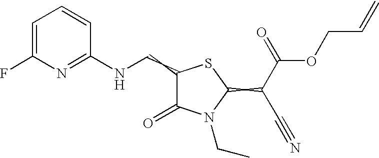 Cyano-[3-ethyl-5-[1-(6-fluoro- pyridin-2-ylamino)-meth-(E/Z)- ylidene]-4-oxo-thiazolidin-(2-(E or Z))-ylidene]-acetic acid allyl ester | (DMSO-d6, stored via K₂CO₃, primary isomer): δ = 1.28 (t, 3H); 4.20 (q, 2H); 4.72 (d, 2H); 5.20-5.46 (m, 2H); 5.84-6.05 (m, 1H); 6.77 (d, 1H); 6.98 (d, 1H); 7.90 (q, 1H); 8.49 (d, 1H); 11.26 (s, 1H) ppm. | 374.397/375 | INTT5/INET5 |
| INTE69 | 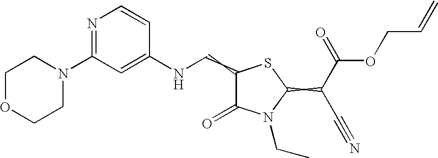 Cyano-[3-ethyl-5-[1-(2- morpholin-4-yl-pyridin-4- ylamino)-meth-( E/Z)-ylidene]-4- oxo-thiazolidin-(2-(E or Z))- ylidene]-acetic acid allyl ester | (DMSO-d6, stored via K2CO3, primary isomer): δ = 1.24 (t, 3H); 3.40 (m, 4H); 3.68 (m, 4H); 4.20 (q, 2H); 4.64 (d, 2H); 5.14-5.41 (m, 2H); 5.86-6.00 (m, 1H); 6.62 (m, 2H); 7.96 (d, 1H); 8.46 (s, 1H); 10.42 (s, 1H) ppm. | 441.513/442 | INTT5 + INT122/INTE5 |
| INTE70 | 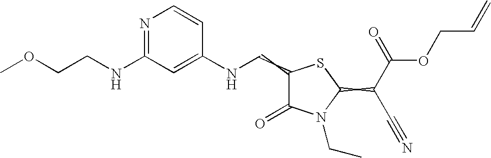 Cyano-[3-ethyl-5-[1-[2-(2- methoxy-ethylamino)-pyridin-4- ylamino]-meth-(E/Z)-ylidene]-4- oxo-thiazolidin-(2-(E or Z))- ylidene]-acetic acid allyl ester | (DMSO-d6, stored via K2CO3, primary isomer): δ = 1.40 (t, 3H); 3.38 (s, 3H); 3.42 (m, 2H); 3.56 (m, 2H); 4.4 (q, 2H); 4.78 (d, 2H) 5.24-5.48 (m, 2H); 5.86 (m, 1H); 6.32 (m, 1H); 7.60 (d, 1H); 7.76 (d, 1H); 8.00 (d, 1H); 10.42 (s, 1H) ppm. | 429.501/430 | INTT5/INTE5 |
| INTE71 | 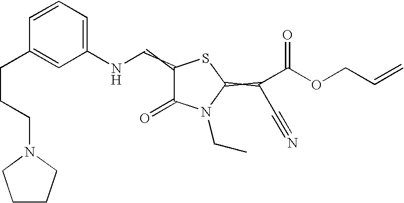 Cyano-[3-ethyl-4-oxo-5-[1-[3-(3- pyrrolidin-1 -yl-propyl)- phenylamino]-meth-(E/Z)- ylidene]-thiazolidin-(2-(E or Z))- ylidene]-acetic acid allyl ester | (DMSO-d6, stored via K₂CO₃, primary isomer): δ = 1.18 (t, 3H); 1.67 (m, 4H); 1.72 (m, 2H); 2.42-2.50 (m, 6H); 2.55 (t, 2H); 4.21 (q, 2H); 4.67 (d, 2H); 5.22 (d, 2H); 5.34 (d, 2H); 5.87-6.01 (m, 1H); 6.89 (d, 1H); 7.08 (d, 1H); 7.14 (s, 1H); 7.21 (t, 1H); 8.18 (s, 1H); 10.52 (s, 1H) ppm. | | INTT5/INTE2 |
| INTE72 | 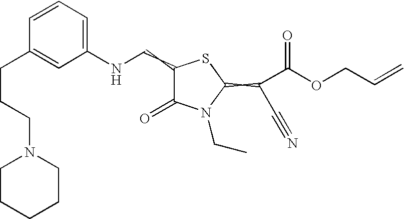 Cyano-[3-ethyl-4-oxo-5-[1-[3-(3- piperidin-1-yl-propyl)- phenylamino]-meth-(E/Z)- ylidene]-thiazolidin-(2-(E or Z))- ylidene]-acetic acid allyl ester | (DMSO-d6, stored via K₂CO₃, primary isomer): δ = 1.20 (t, 3H); 1.34 (m, 2H); 1.46 (m, 4H); 1.70 (m, 2H); 2.20-2.37 (m, 6H); 2.53 (t, 2H); 4.20 (q, 2H); 4.67 (d, 2H); 5.22 (d, 2H); 5.34 (d, 2H); 5.87-6.01 (m, 1H); 6.88 (d, 1H); 7.08 (d, 1H); 7.12 (s, 1H); 7.21 (t, 1H); 8.16 (s, 1H); 10.45 (s, 1H) ppm. | | INTT5/INTE2 |
| INTE73 | 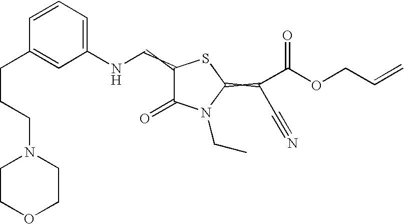 Cyano-[3-ethyl-5-[1-[3-(3- morpholin-4-yl-propyl)- phenylamino]-meth-(E/Z)- ylidene]-4-oxo-thiazolidin-(2-(E or Z))-ylidene]-acetic acid allyl ester | (DMSO-d6, stored via K₂CO₃, primary isomer): δ = 1.21 (t, 3H); 1.69 (m, 2H); 2.22 (t, 2H); 2.30 (m, 4H); 2.53 (t, 2H); 3.54 (t, 4H); 4.21 (q, 2H); 4.67 (d, 2H); 5.22 (d, 2H); 5.34 (d, 2H); 5.88-6.01 (m, 1H); 6.91 (d, 1H); 7.08 (d, 1H); 7.15 (s, 1H); 7.21 (t, 1H); 8.18 (m, 1H); 10.46 (m, 1H) ppm. | | INTT5/INTE2 |
| INTE74 | 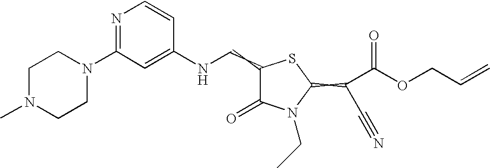 Cyano-[3-ethyl-5-[1-[2-(4-methyl- piperazin-1-yl)-pyridin-4- ylamino]-meth-(E/Z)-ylidene]-4- oxo-thiazolidin-(2-(E oder Z))- ylidene]-acetic acid allyl ester | | 454.555/455 | INTT5 + INT124/INTE5 |
| INTE75 | 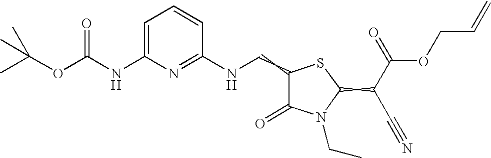 [5-[1-(6-tert- Butoxycarbonylamino-pyridin-2- ylamino)-meth-(E/Z)-ylidene]-3- ethyl-4-oxo-thiazolidin-(2-(E oder Z))-ylidene]-cyano-acetic acid allyl ester | | 471.539/472 | INTT5/INTE2 |
| INTE76 | 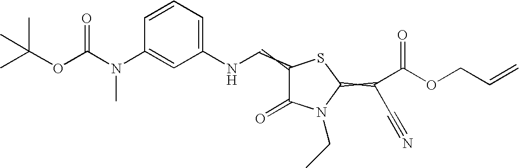 [5-[1-[3-(tert-Butoxycarbonyl- methyl-amino)-phenylamino]- meth-(E/Z)-ylidene]-3-ethyl-4- oxo-thiazolidin-(2-(E oder Z))- ylidene]-cyano-acetic acid allyl ester | | 484/485 | INTT5/INTE1 |

### Intermediate INTE77

### 4-[2-(3-{[2-[1-Allyloxycarbonyl-1-cyano-meth-(E or Z)-ylidene]-3-ethyl-4-oxo-thiazolidin-(5-(E/Z))-ylidenemethyl]-amino}-phenoxy)-acetyl]-piperazine-1-carboxylic acid tert-butyl ester

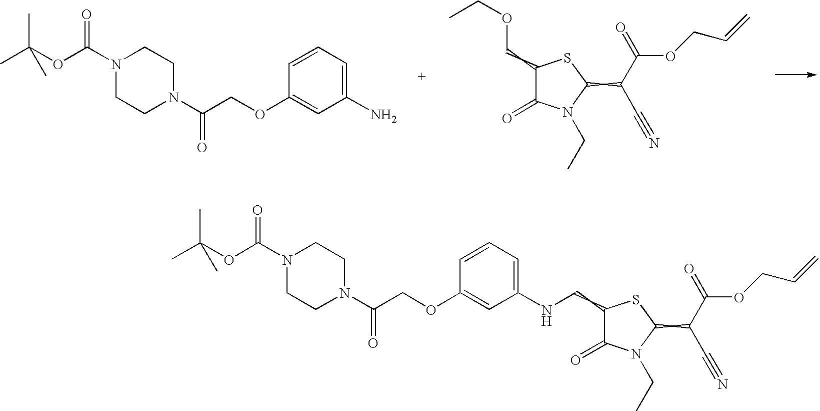

Dissolve 4.8 g of the compound described under INT77 and 4.4 g of the compound described under INTT5 in ethanol (140 ml) and stir under argon for three hours at 95° C. bath temperature. The condensation, thus arisen, is siphoned off and washed with ethanol. The compound in the title (5.7 g) is obtained in a 67% yield. The raw product is used at the next level without further purification.

¹H-NMR (DMSO-d6, stored over K₂CO₃, primary isomer) δ=1.26 (t, 3H); 1.40 (s, 2H); 3.32 (m, 4H); 3.45 (m, 4H); 4.28 (m, 2H); 4.72 (d, 2H); 4.89 (s, 2H); 5.29 (dd, 1H); 5.40 (dd, 1H); 5.99 (m, 1H); 6.68 (dd, 1H); 6.90 (s, 1H); 6.93 (d, 1H); 7.28 (t, 1H); 8.21 (d, 1H); 10.47 (d, 1H) ppm.

### Intermediate INTE78

### Cyano-[3-ethyl-4-oxo-5-[1-[3-(2-oxo-2-piperazin-1-yl-ethoxy)-phenylamino]-meth-(E/Z)-ylidene]-thiazolidin-(2-(E or Z))-ylidene]-acetic acid allyl ester

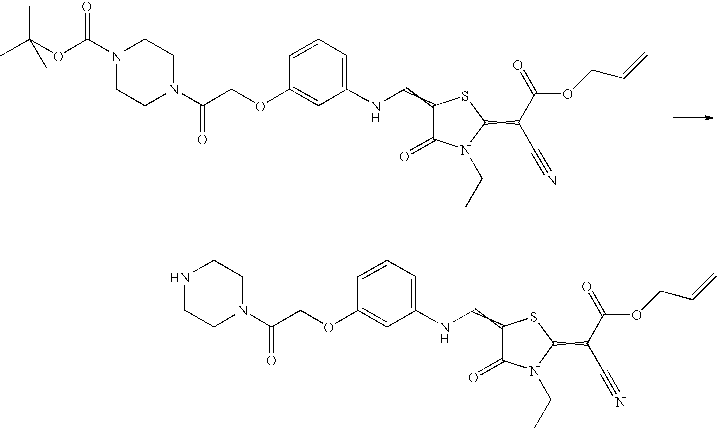

Dissolve 2.99 g of the compound described under INTE77 in dichloro-methane (100 ml) and slowly add trifluoro-acetic acid (10 ml) to it. Stir for 2.5 hours under argon at room temperature and then end the reaction through the addition of a 10% watery sodium carbonate solution (approx. 170 ml). Then extract the reaction mixture with dichloro-methane (3×100 ml), wash the unified organic phases with a sodium chloride solution (1×100 ml) and dry following this over sodium sulfate. After distilling off the solvent on the rotation vaporizer, the compound in the title (2 g) is obtained in an 80% yield. The raw product was used at the next level without further purification.

¹H-NMR (DMSO-d6, stored over K₂CO₃, primary isomer) δ=1.23 (m, 3H); 2.68 (m, 2H); 2.71 (m, 2H); 4.25 (m, 2H); 4.73 (m, 2H); 4.82 (s, 2H); 5.29 (dd, 1H); 5.39 (dd, 1H); 5.99 (m, 1H); 6.64 (dd, 1H); 6.88 (s, 1H); 6.91 (d, 1H); 7.27 (t, 1H); 8.22 (s, 1H) ppm.

### Intermediate INTE79

### [5-[1-{3-[2-(4-Benzyl-piperazin-1-yl)-2-oxo-ethoxy]-phenylamino}-meth-(E/Z)-ylidene]-3-ethyl-4-oxo-thiazolidin-(2-(E or Z))-ylidene]-cyano-acetic acid allyl ester

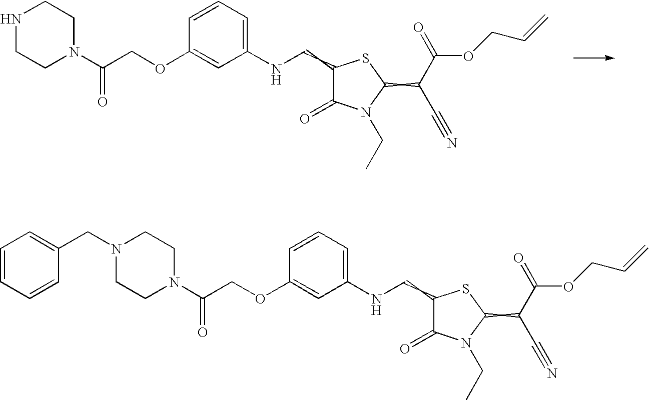

Suspend 2.9 g of the compound described under INTE78 and 0.92 ml benzaldehyde in methanol (240 ml) and add acetic acid (24 ml) and sodium cyanoborohydride (0.7 g) to it at room temperature. Stir the residue at room temperature for 5 hours under argon, neutralize the reaction mixture through the addition of sodium carbonate and siphon off the condensation thus arisen. The compound in the title (2.54 g) is obtained in a 71% yield. The product is used at the next level without further purification.

¹H-NMR (DMSO-d6, stored over K₂CO₃, primary isomer) ¹H-NMR δ=1.29 (m, 3H); 2.32 (m, 2H); 2.41 (m, 2H); 3.43 (m, 4H); 4.26 (m, 2H); 4.72 (d, 2H); 4.86 (s, 2H); 5.29 (d, 1H); 5.40 (d, 1H); 6.00 (m, 1H); 6.68 (dd, 1H); 6.89 (s, 1H); 6.92 (d, 1H); 7.30 (m, 6H); 8.21 (d, 1H); 10.50 (d, 1H) ppm.

### 4. Synthesis of Acid-Intermediates

### Intermediate INTA1

### Manufacturing Variant 1

### Cyano-[3-ethyl-4-oxo-5-[1-[3-(2-pyrrolidin-1-yl-ethyl)-phenylamino]-meth-(E/Z)-ylidene]-thiazolidin-(2-(E or Z))-ylidene]-acetic acid

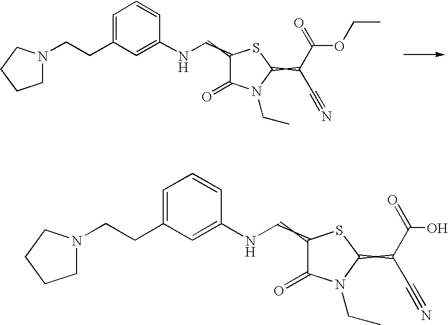

Pre-place 1.1 g potassium-(tert)-butylate in 50 ml tetrahydrofurane at 0° C. and add 45 μl water. Add 540 mg of the compound described under Intermediate INTEL) and stir for 30 minutes at 0° C., and for 20 hours at room temperature. At 0° C., add 0.25 ml triethylamine and 10.5 ml two molar salt acid (hydrochloric acid) in diethylether and stir at room temperature for an hour. Allow the solvent to condense under high vacuum and use the residue without any further purification.

MW: 412.51; MS (ESI) [M+1]⁺: 413

### Manufacturing Variant 2

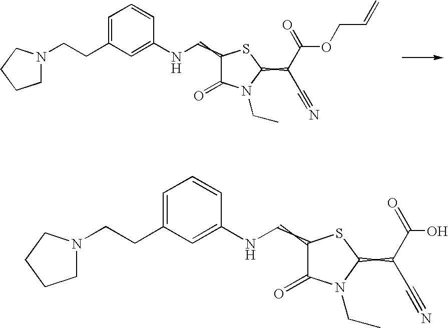

Dissolve 300 mg of the compound described under INTE2), 80 mg Pd (PPh₃)₄ and 0.6 ml morpholine in 18 ml tetrahydrofurane and stir for 15 hours. After an addition of 40 ml diethylether, filter the solid thus obtained, dry in vacuum and dissolve in 10 ml dimethylformamide. Add the solution to a suspension of 770 mg PL-MIA Resin of the firm Polymer Laboratories GmbH in 5 ml dimethylformamide and stir for 15 hours at room temperature. Filter the reaction mixture and allow the solvent to condense under high vacuum. 280 mg of the compound in the title is obtained as a raw product.

¹H-NMR (DMSO-d6, stored over K₂CO₃): δ=1.20 (t, 3H); 1.88 (m, 4H); 2.50 (m, 4H); 3.09 (m, 2H); 3.20 (m, 2H); 4.20 (q, 2H); 6.93 (d, 1H); 7.04-7.12 (m, 2H); 7.23 (t, 1H); 7.88 (s, 1H); 9.97 (s, 1H) ppm.

### Intermediate INTA2

### Cyano-[3-ethyl-5-[1-(2-ethylamino-pyridin-4-ylamino)-meth-(E/Z)-ylidene]-4-oxo-thiazolidin-(2-(E or Z))-ylidene]-acetic acid

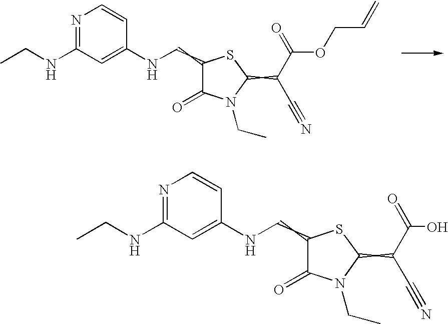

Dissolve 1.2 g of the compound described under INTE4), 350 mg Pd (PPh₃)₄ and 2.6 ml morpholine in 60 ml tetrahydrofurane and stir for an hour at room temperature. After the addition of 40 ml of hexane, filter the solid obtained, dry in vacuum and dissolve in 20 ml dimethylformamide. Add the solution to a suspension of 6.0 g PL-MIA Resin of the firm Polymer Laboratories GmbH in 30 ml dimethylformamide and stir for 15 hours at room temperature. Filter the reaction mixture and allow the solvent to condense under high vacuum. 970 mg of the compound in the title is obtained as a raw product.

MW: 359.41; MS (ESI) [M+1]⁺: 360 ¹H-NMR (DMSO-d6, stored over K₂CO₃): δ=1.11 (t, 3H); 1.22 (t, 3H); 3.23 (m, 2H); 4.22 (q, 2H); 6.25 (s, 1H); 6.42 (d, 1H); 6.54 (s, b, 1H); 7.81 (d, 1H); 7.95 (s, 1H); 10.20 (s, 1H) ppm.

### Intermediate INTA3

### Cyano-[5-[1-[6-(2,2-dimethyl-propionylamino)-pyridin-2-ylamino]-meth-(E/Z)-ylidene]-3-ethyl-4-oxo-thiazolidin-(2-(E or Z))-ylidene]-acetic acid

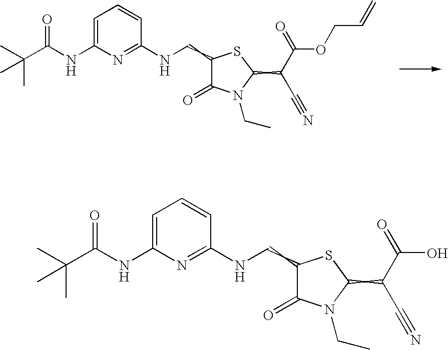

Dissolve 2.2 g of the compound described under INTE5), 560 mg Pd (PPh₃)₄ and 4.2 ml morpholine in 110 ml tetrahydrofurane and stir for an hour at room temperature. After the addition of 50 ml of hexane, filter the excluded solid, dry in vacuum and dissolve in 25 ml dimethylformamide. Add the solution to a suspension of 9.6 g PL-MIA Resin of the firm Polymer Laboratories GmbH in 50 ml dimethylformamide and stir for 15 hours at room temperature. Filter the reaction mixture and allow the solvent to condense under high vacuum. 2.1 g of the compound in the title is obtained as a raw product.

MW: 415.47; MS (ESI) [M+1]⁺: 416 ¹H-NMR (DMSO-d6, stored over K₂CO₃): δ=1.15-1.30 (m, 12H); 4.23 (q, 2H); 6.80 (m, 1H); 7.64-7.74 (m, 2H); 8.73 (d, 1H); 9.68 (s, 1H); 10.68 (d, 1H) ppm.

The following compounds are manufactured in addition to the process described above.

| | | | Molecular | |
|---|---|---|---|---|
| | | | Weight/ | |
| | | | MS | Educt/ |
| Example | | | (ESI) | Additional |
| no. | Structure and name | ¹H-NMR | [M + 1]⁺ | synthesis |
| INT A4 | 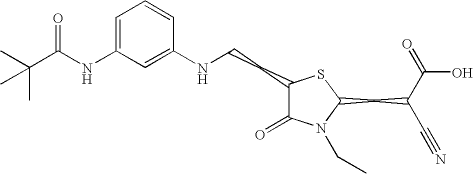 Cyano-[5-[1-[3-(2,2-dimethyl- propionylamino)-phenylamino]- meth-(E/Z)-ylidene]-3-ethyl-4- oxo-thiazolidin-(2-(E or Z))- ylidene]-acetic acid | | MW: 414.49 MS (ESI) [M + 1]⁺: 415 | INTE6/ INTA3 |
| INT A5 | 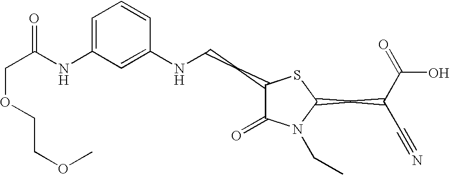 Cyano-[3-ethyl-5-[1-{3-[2-(2- methoxy-ethoxy)-acetylamino]- phenylamino}-methyl(E/Z)- ylidene]-4-oxo-thiazolidin-(2-(E or Z))-ylidene]-acetic acid | (DMSO-d6, stored via K₂CO₃): δ = 1.22 (t, 3H); 3.30 (s, 2H); 3.54 (m, 2H); 3.68 (m, 2H); 4.09 (s, 2H); 4.23 (q, 2H); 7.01 (m, 1H); 7.22-7.32 (m, 2H); 7.75 (s, 1H); 8.04 (d, 1H); 9.71 (s, 1H); 10.50 (d, 1H) ppm. | MW: 446.48 MS (ESI) [M + 1]⁺: 447 | INTE7/ INTA3 |
| INT A6 | 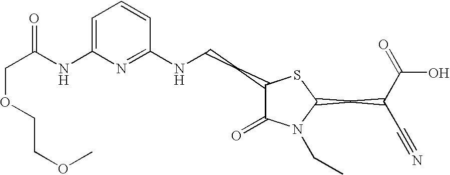 Cyano-[3-ethyl-5-[1-{6-[2-(2- methoxy-ethoxy)-acetylamino]- pyridin-2-ylamino}-meth-(E/Z)- ylidene]-4-oxo-thiazolidin-(2-(e or Z))-ylidene]-acetic acid | (DMSO-d6, stored via K₂CO₃): δ = 1.23 (t, 3H); 3.34 (s, 3H); 3.51 (m, 2H); 3.69 (m, 2H); 4.15 (s, 2H); 4.22 (q, 2H); 6.81 (dd, 1H); 7.69-7.78 (m, 2H); 7.95 (s, 1H); 8.64 (d, 1H); 9.98 (s, 1H); 10.73 (d, 1H) ppm. | MW: 447.47 MS (ESI) [M + 1]⁺: 448 | INTE9/ INTA3 |
| INT A7 | 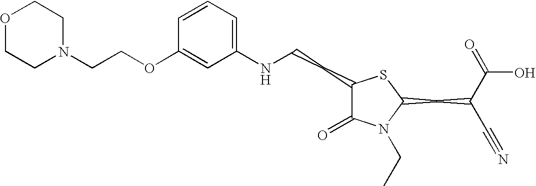 Cyano-[3-ethyl-5-[1-[3-(2- morpholin-4-yl-ethoxy)- phenylamino]-meth-(E/Z)- ylidene]-4-oxo-thiazolidin-(2-(E or Z))-ylidene]-acetic acid | | 444.51/ 4445 | INTE21/ INTA3 |
| INT A8 | 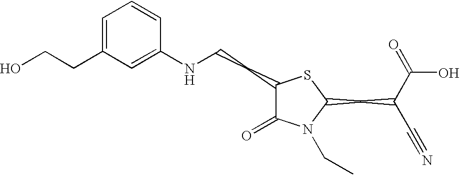 Cyano-[3-ethyl-5-[1-[3-(2- hydroxy-ethyl)-phenylamino]- meth-(E/Z)-ylidene]-4-oxo- thiazolidin-(2-(E or Z))-ylidene]- acetic acid | (DMSO-d6, stored via K₂CO₃) (selected signals): δ = 1.23 (t, 3H); 2.71 (t, 2H); 3.61 (t, 2H); 4.23 (q, 2H); 4.68 (b, 1H); 6.91 (d, 1H); 7.10 (d, 1H); 7.16 (s, 1H); 7.23 (t, 1H); 8.05 (d, 1H); 10.23 (d, 1H) ppm. | 359.41/ 360 | INTE16/ INTA3 |
| INT A9 | 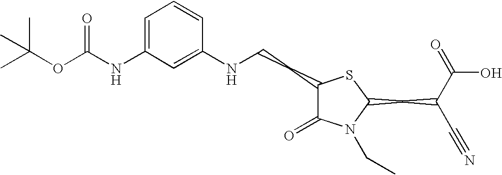 [5-[1-(3-tert- Butoxycarbonylamino- phenylamino)-meth-(E/Z)- ylidene]-3-ethyl-4-oxo- thiazolidin-(2-(E or Z))-ylidene]- cyano-acetic acid | (DMSO-d6, stored via K₂CO₃) (selected signals): δ = 1.20 (t, 3H); 1.48 (s, 9H); 4.20 (q, 2H); 6.83 (d, 1H); 7.03 (d, 1H); 7.18 (t, 1H); 7.51 (s, 1H); 7.88 (d, 1H); 9.40 (s, 1H); 10.16 (d, 1H) ppm. | 430.49/ 431 | INTE17/ INTA3 |
| INT A10 | 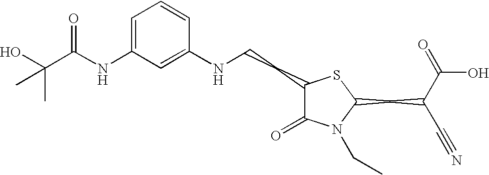 Cyano-[3-ethyl-5-[1-[3-(2- hydroxy-2-methyl- propionylamino)-phenylamino]- meth-(E/Z)-ylidene]-4-oxo- thiazolidin-(2-(E or Z))-ylidene]- acetic acid | | 416.46/ 417 | INTE3/ INTA3 |
| INT A11 | 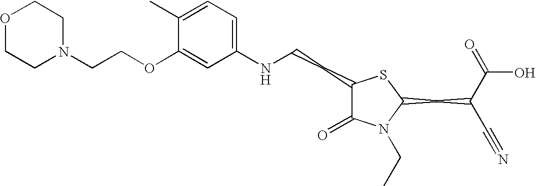 Cyano-[3-ethyl-5-[1-[4-methyl-3- (2- Morpholin-4-yl-ethoxy)- phenylamino]- meth-(E/Z)-ylidene]-4-oxo- thiazolidin-(2-(E or Z))-ylidene]- acetic acid | ¹H-NMR (DMSO-d6, 300 MHz) (selected peaks) δ = 1.25 (m, 3H); 2.09 (s, 3H); 2.58 (m, 4H); 2.81 (m, 2H); 3.61 (m, 4H); 4.15 (m, 2H); 4.26 (m, 2H); 6.81 (dd, 1H); 6.92 (s, 1H); 7.01 (d, 1H); 8.20 (d, 1H); 10.35 (d, 1H); 11.08 (s, 1H). | MW: 458.536 MS(ESI) [M + 1]⁺: 459 | INTE26/ INTA3 |
| INT A12 | 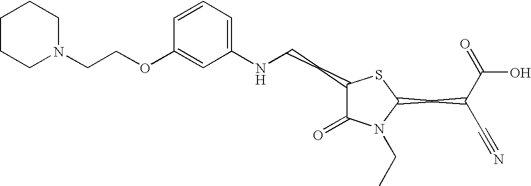 Cyano-[3-ethyl-4-oxo-5-[1-[3-(2- piperidin-1-yl-ethoxy)- phenylamino]-meth-(E/Z)- ylidene]-thiazolidin-(2-(E or Z))- ylidene]-acetic acid | ¹H-NMR (DMSO-d6, 300 MHz) (selected peaks) δ = 1.19 (m, 3H); 1.47 (m, 2H); 1.66 (m, 4H); 2.88 (m, 4H); 3.10 (m, 2H); 4.12 (m, 2H); 4.21 (m, 2H); 6.62 (dd, 1H); 6.82 (m, 2H); 7.21 (m, 1H); 8.00 (d, 1H); 10.00 (d, 1H). | MW: 442.537 MS (ESI) [M + 1]⁺: 443 | INTE25/ INTA3 |
| INT A13 | 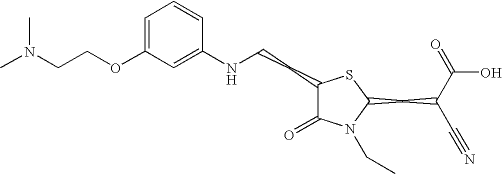 Cyano-[5-[1-[3-(2- dimethylamino-ethoxy)- phenylamino]-meth-(E/Z)- ylidene]-3-ethyl-4-oxo- thiazolidin-(2-(E or Z))-ylidene]- acetic acid | ¹H-NMR (CDCl₃, 300 MHz) (selected peaks) δ = 1.23 (m, 3H); 2.88 (s, 6H); 4.23 (m, 2H); 4.37 (m, 2H); 6.73 (dd, 1H); 6.97 (m, 2H); 7.30 (m, 1H); 8.20 (s, 1H). | MW: 402.472 MS (ESI) [M + 1]⁺: 403 | INTE20/ INTA3 |
| INT A14 | 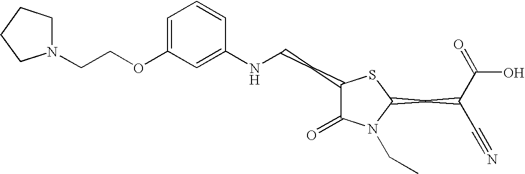 Cyano-[3-ethyl-4-oxo-5-[1-[3-(2- pyrrolidin-1-yl-ethoxy)- phenylamino]-mEth-(E/Z)- ylidene]-thiazolidin-(2-(Z or E))- ylidene]-acetic acid | | MW: 428.51 MS (ESI) [M + 1]⁺: 429 | INTE24/ INTA3 |
| INT A15 | 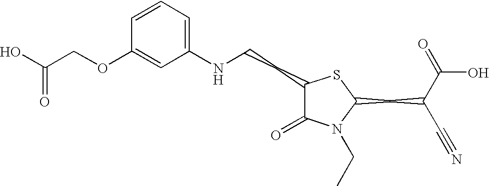 [5-[1-(3-Carboxymethoxy- phenylamino)-meth-(E/Z)- ylidene]-3-ethyl-4-oxo- thiazolidin-(2-(E or Z))-ylidene]- cuano-acetic acid | | MW: 389.39 MS (ESI) [M + 1]⁺: 390 | INTE23/ INTA3 |
| INT A16 | 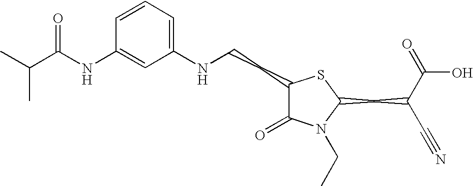 Cyano-[3-ethyl-5-[1-(3- isobutyryl-amino-phenylamino)- meth-(E/Z)-ylidene]-4-oxo- thiazolidin-(2-(E or Z))-ylidene]- acetic acid | | MW: 400.460/ MS (ESI) [M + 1]⁺: 401 | INTE28/ INTA3 |
| INT A17 | 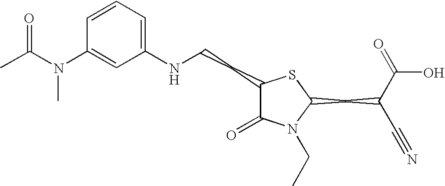 [5-[1-[3-(Acetyl-methyl-amino)- phenylamino]-meth-(E/Z)- ylidene]-3-ethyl-4-oxo- thiazolidin-(2-(E or Z))-ylidene]- cyano-acetic acid | | MW: 386.433/ MA (ESI) [M + 1]⁺: 387 | INTE30/ INTA3 |
| INT A18 | 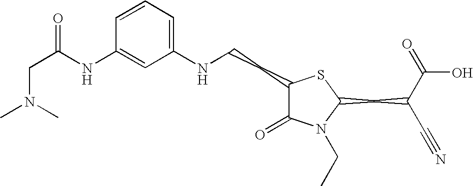 Cyano-[5-[1-[3-(2- dimethylamino-acetylamino)- phenylamino]-meth-(E/Z)- ylidene]-3-ethyl-4-oxo- thiazolidin-(2-(E or Z))-ylidene]- acetic acid | | MW: 415.474/ MS (ESI) [M + 1]⁺: 416 | INTE32/ INTA3 |
| INT A19 | 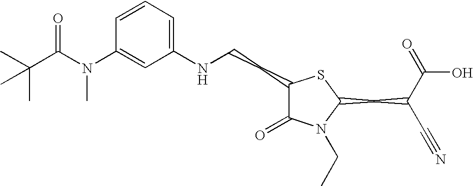 Cyano-[5-[1-{3-[(2,2-dimethyl- propionyl)-methyl-amino]- phenylamino}-meth-(E/Z)- ylidene]-3-ethyl-4-oxo- thiazolidin-(2-(E or Z))-ylidene]- acetic acid | | MW: 428.514/ MS (ESI) [M + 1]⁺: 429 | INTE34/ INTA3 |
| INT A20 | 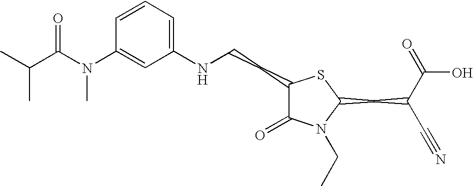 Cyano-[3-ethyl-5-[1-[3- (isobutyryl- methyl-amino)-phenylamino]- meth-(E/Z)-ylidene]-4-oxo- thiazolidin-(2-(E or Z))-ylidene]- acetic acid | | MW: 414.486/ MS (ESI) [M + 1]⁺: 415 | INTE36/ INTA3 |
| INT A21 | 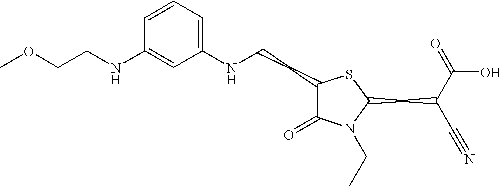 Cyano-[3-ethyl-5-[1-[3-(2- methoxy- ethylamino)-phenylamino]- meth-(E/Z)-ylidene]-4-oxo- thiazolidin-(2-(E or Z))-ylidene]- acetic acid | | MW: 388.448/ MS (ESI) [M + 1]⁺: 389 | INTE39/ INTA3 |
| INT A22 | 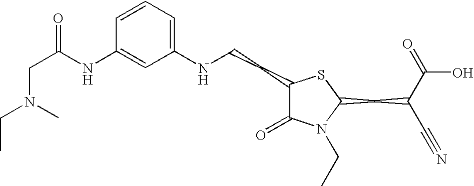 Cyano-[3-ethyl-5-[1-{3-[2-(ethyl- methyl-amino)-acetylamino]- phenylamino}-meth-(E/Z)- ylidene]-4-oxo-thiazolidin-(2Z)- ylidene]-acetic acid | | MW: 429.501/ MS (ESI) [M + 1]⁺: 430 | INTE41/ INTA3 |

### Intermediate INTA23

### [5-[1-{3-[2-(4-Benzyl-piperazin-1-yl)-2-oxo-ethoxy]-phenylamino}-meth-(E/Z)-ylidene]-3-ethyl-4-oxo-thiazolidin-(2-(E or Z))-ylidene]-cyano-acetic acid

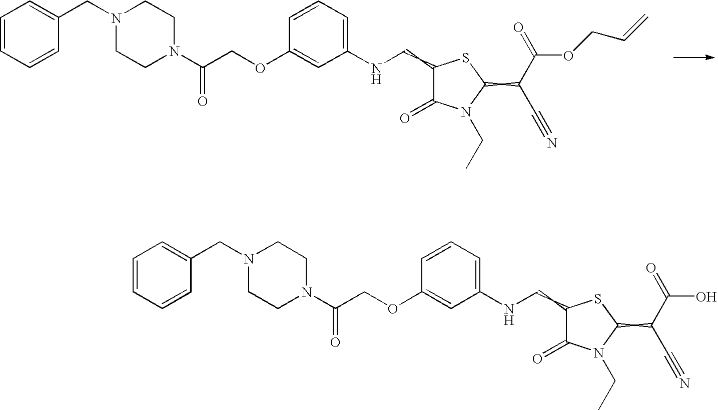

Suspend 2.5 g of the compound described under INTE79 in THF (320 ml) and add to barbituric acid (0.6 g) and Pd(PPh₃)₄ (0.49 g). Stir the reaction mixture overnight, press on the rotary evaporator until a precipitation occurs and extract the resulting condensation. The compound in the title (522 mg) is obtained in a 23% yield. The product is used at the next level without further purification.

EI-MS=548.

The following compounds are manufactured in addition to the process described above.

| | | | Molecular | |
|---|---|---|---|---|
| Ex- | | | Weight/ | |
| am- | | | MS | Educt/ |
| ple | | | (ESI) | Additional |
| no. | Structure and name | ¹H-NMR | [M + 1]⁺ | synthesis |
| INT A24 | 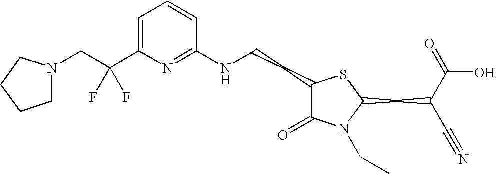 Cyano-[5-[1-[6-(1,1-difluoro-2- pyrrolidin-1-yl-ethyl)-pyridin-2- ylamino]-meth-(E/Z)-ylidene]-3- ethyl-4-oxo-thiazolidin-(2-(E or Z))-ylidene]-acetic acid | (DMSO-d6, stored via K₂CO₃, selected signals): δ = 1.18 (t, 3H); 1.58 (b, 4H); 2.50 (b, 4H); 3.19-3.27 (m, 2H); 4.15 (q, 2H); 7.13 (d, 1H); 7.20 (d, 1H); 7.80 (t, 1H); 8.40 (s, b, 1H); 10.50 (s, b, 1H) ppm | | INTE49/ INTA3 |
| INT A25 | 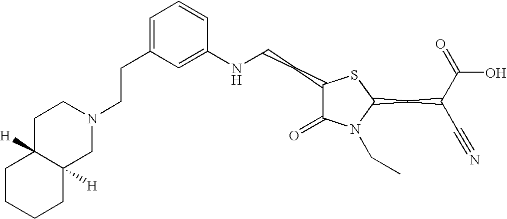 Cyano-[3-ethyl-5-({3- [(4aR,8aS)-2- (octahydro-isoquinolin-2-yl)- ethyl]-phenylamino}-meth-(E/Z)- ylidene-4-oxo-thiazolidin-(2-(E or Z))-ylidene]-acetic acid | | 480.63/ 481 | INTE43/ INTA3 |
| INT A26 | 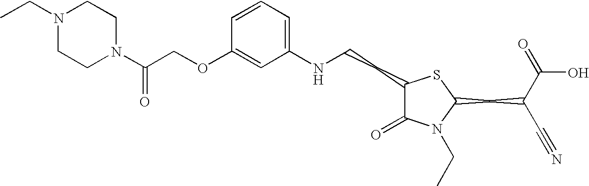 Cyano-[3-ethyl-5-[1-{3-[2-(4- ethyl-piperazin-1-yl)-2-oxo- ethoxy]-phenylamino}-meth- (E/Z)-ylidene]-4-oxo-thiazolidin- (2-(E or Z))-ylidene]-acetic acid | (DMSO-d6, stored via K₂CO₃, primary isomer): δ = 1.00 (m, 3H); 1.20 (m, 3H); 1.72 (m, 2H); 2.39 (m, 6H); 3.57 (m, 2H); 4.20 (m, 2H); 4.81 (s, 2H); 6.60 (dd, 1H); 6.82 (s, 1H); 6.88 (d, 1H); 7.20 (t, 1H); 8.09 (d,1H); 10.29 (d, 1H)ppm. | 485.57/ 486 | INTE50/ INTA23 |
| INT A27 | 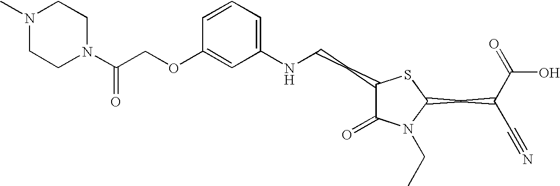 Cyano-[3-ethyl-5-[1-{3-[2-(4- methyl-piperazin-1-yl)-2-oxo- ethoxy]-phenylamino}-meth- (E/Z)-ylidene]-4-oxo-thiazolidin- (2-(E or Z))-ylidene]-acetic acid | (DMSO-d6, stored via K₂CO₃, primary isomer): δ = 1.21 (t, 3H); 2.22 (m, 2H); 2.41 (m, 2H); 3.43 (m, 4H); 4.19 (m, 2H); 4.81 (s, 2H); 6.60 (dd, 1H); 6.82 (s, 1H); 6.88 (d, 1H); 7.20 (t, 1H); 8.08 (d, 1H); 10.29 (d, 1H) ppm. | 471.54 472 | INTE51/ INTA23 |
| INT A28 | 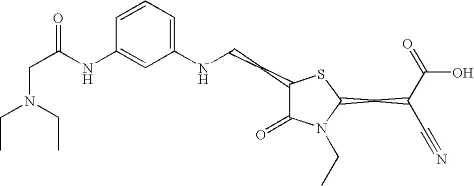 Cyano-[5-[1-[3-(2-diethylamino-0 acetylamino)-phenylamino]- meth-(E/Z)-ylidene]-3-ethyl-4- oxo-thiazolidin-(2--(E or Z))- ylidene]-acetic acid | | MW: 443.53/ MS (ESI) [M + 1]⁺: 444 | INTE53/ INTA3 |
| INT A29 | 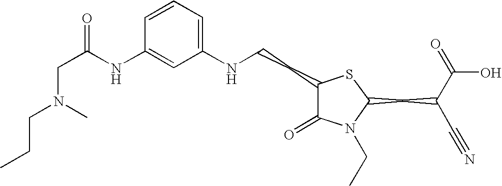 Cyano-[3-ethyl-5-[1-{3-[2- (methyl-propyl-amino)-acetyl- amino]-phenylamino}-meth- (E/Z)-ylidene]-4-oxo-thiazol- idin-(2-(E or Z))-ylidene]-acetic acid | | MW: 443.53/ MS (ESI) [M + 1]⁺: 444 | INTE55/ INTA3 |
| INT A30 | 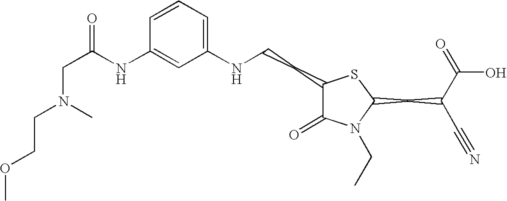 Cyano-[3-ethyl-5-[1-(3-{2-[(2- methoxy-ethyl)-methyl-amino]- acetylamino}-phenylamino)- meth-(E/Z)-ylidene]-4- oxo-thiazolidin-(2-(E or Z))- ylidene]-acetic acid | | MW: 459.53/ MS (ESI) [M + 1]⁺: 460 | INTE59/ INTA3 |
| INT A31 | 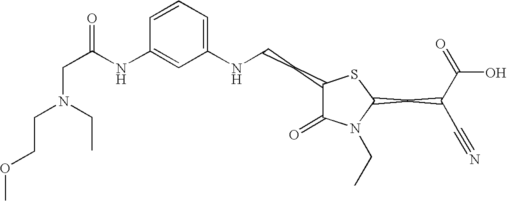 Cyano-[3-ethyl-5-[1-(3-{2-[ethyl- (2-methoxy-ethyl)-amino]- acetylamino}-phenylamino)- meth-(E/Z)-ylidene]-4-oxo- thiazolidin-(2-(E or Z))-ylidene]- acetic acid | | MW: 473.55/ MS (ESI) [M + 1]⁺: 474 | INTE61/ INTA3 |
| INT A32 | 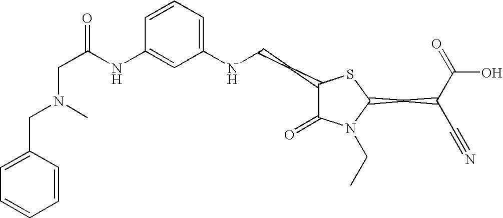 [5-[1-{3-[2-(Benzyl-methyl- amino)-acetylamino]-phenyl- amino}-meth-(E/Z)-ylidene]-3- ethyl-4-oxo-thiazol-idin-(2-(E or Z))-ylidene]-cyano acetic acid | | MW: 491.57/ MS (ESI) [M + 1]⁺: 492 | INTE63/ INTA3 |
| INT A33 | 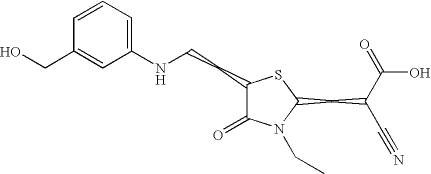 Cyano-[3-ethyl-5-[1-(3- hydroxymethyl-phenylamino)- meth-(E/Z)-ylidene]-4-oxo- thiazolidin-(2-(E or Z))-ylidene]- acetic acid | (DMSO-d6, stored via K₂CO₃, primary isomer): δ = 1.26 (t, 3H); 4.25 (q, 2H); 4.51 (s, 2H); 5.27 (s, 1H); 7.04 (d, 1H); 7.18 (d, 2H); 7.27-7.34 (m, 2H); 8.17 (d, 1H); 10.53 (d, 1H); 13.03 (s, 1H) ppm. | | INTE12/ INTA23 |
| INT A34 | 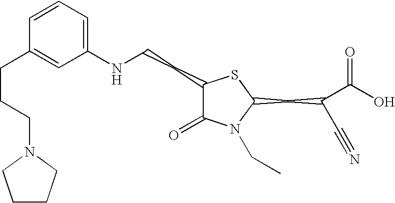 Cyano-[3-ethyl-4-oxo-5-[1-[3-(3- pyrrolidin-1-yl-propyl)- phenylamino]-meth-(E/Z)- ylidene]-thiazolidin-(2-(E or Z))- ylidene]-acetic acid | (DMSO-d6, stored via K₂CO₃, primary isomer): δ = 1.22 (t, 3H); 1.94 (m, 4H); 2.05 (m, 2H); 2.54-2.62 (m, 6H); 2.70 (t, 2H); 4.25 (q, 2H); 6.98 (d, 1H); 7.10-7.18 (m, 2H); 7.32 (t, 1H); 7.95 (m, 1H); 10.08 (m, 1H); 11.60 (m, 1H) ppm. | | INTE71/ INTA23 |
| INT A35 | 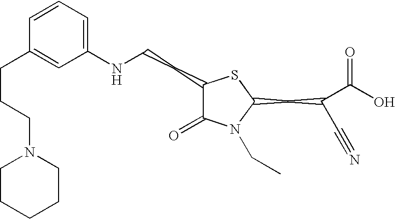 Cyano-[3-ethyl-4-oxo-5-[1-[3-(3- piperidin-1-yl-propyl)- phenylamino]-meth-(E/Z)- ylidene]-thiazolidin-(2-(E or Z))- ylidene]-acetic acid | (DMSO-d6, stored via K₂CO₃, primary isomer): δ = 1.15 (t, 3H); 1.46 (m, 2H); 1.62-1.70 (m, 4H); 1.94 (m, 2H); 2.47-2.61 (m, 4H); 2.78 (m, 2H); 2.92 (m, 2H); 4.15 (q, 2H); 6.86 (d, 1H); 7.00-7.08 (m, 2H); 7.20 (t, 1H); 7.86 (m, 1H); 9.98 (m, 1H); 11.48 (m, 1H) ppm. | | INTE72/ INTA23 |
| INT A36 | 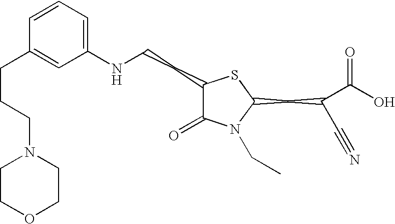 Cyano-[3-ethyl-5-[1-[3-(3- morpholin-4-yl-propyl)- phenylamino]-meth-(E/Z)- ylidene]-4-oxo-thiazolidin-(2-(E or Z))-ylidene]-acetic acid | (DMSO-d6, stored via K₂CO₃, primary isomer): δ = 1.18 (t, 3H); 1.71 (m, 2H); 2.35 (t, 2H); 2.40-2.58 (m, 6H); 3.52-3.63 (m, 4H); 4.18 (q, 2H); 6.87 (d, 1H); 7.07 (d, 1H); 7.13 (s, 1H); 7.20 (t, 1H); 8.08 (d, 1H); 10.29 (d, 1H); 11.47 (s, 1H) ppm. | | INTE73/ INTA23 |
| INT A37 | 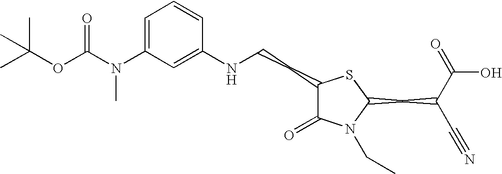 [5-[1-[3-(tert-Butoxycarbonyl- methyl-amino)-phenylamino]- meth-(E/Z)-ylidene]-3-ethyl-4- oxo-thiazolidin-(2-(E or Z))- ylidene]-cyano-acetic acid | | 444/ 445 | INTE76/ INA2 |

### 5. Synthesis of Amides

### Example 1

### 2-Cyano-2-[3-ethyl-4-oxo-5-[1-[3-(2-pyrrolidin-1-yl-ethyl)-phenylamino]-meth-(E/Z)-ylidene]-thiazolidin-(2-(E or Z))-ylidene]-N-(2-hydroxy-1,1-dimethyl-ethyl)-acetamide

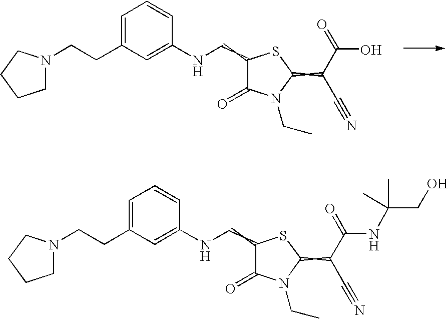

Dissolve 170 mg of the raw product described under intermediate INTA1) (approx. 0.42 mmol) in 10 ml dimethylformamide, add 248 mg sodium hydrogencarbonate, 62 μl 2-amino-2-methyl-propane-1-ol, and 200 mg TBTU and stir for 18 hours at room temperature. Add a semi-saturated sodium hydrogencarbonate solution to the reaction mixture and extract with dichlormethane. Wash the organic solution with saturated sodium chloride, dry over sodium sulfate, press and, after purification through chromatography in silica gel, 61 mg of the compound in the title is obtained as a pH dependent 5-(E/Z)-isomer mixture.

¹H-NMR (DMSO-d6, stored over K₂CO₃, primary isomer): δ=1.30 (t, 3H); 1.36 (s, 6H); 1.74 (m, 4H); 2.54 (m, 4H); 2.69 (m, 2H); 2.79 (m, 2H); 3.43 (d, 2H); 4.27 (q, 2H); 5.27 (t, 1H); 6.74 (s, 1H); 7.00 (d, 1H); 7.18 (d, 1H); 7.25-7.35 (m, 2H); 8.19 (s, 1H); 10.31 (s, 1H) ppm.

### Example 2

### Tetrahydro-pyran-4-carboxylic acid (3-{[2-[1-cyano-1-ethylcarbamoyl-meth-(E or Z)-ylidene]-3-ethyl-4-oxo-thiazolidin-(5-(E/Z))-ylidenemethyl]-amino}-phenyl)-amide

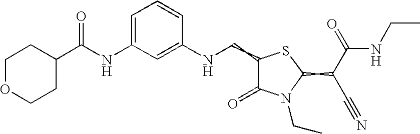

Dissolve 42 mg tetrahydropyran-4-carboxylic acid in 10 ml tetrahydrofurane. At 0° C., add 80 μl triethylamine and 42 μl isobutylchloroformate to it. Stir for 30 minutes at room temperature. Then add 100 mg of the compound described under Example 6). Stir for 12 hours at room temperature. Add a semi-saturated sodium hydrogencarbonate solution to the reaction mixture and extract with dichlormethane. Wash the organic solution with saturated sodium chloride, dry over sodium sulfate, press and, after purification through chromatography in silica gel, 49 mg of the compound in the title is obtained as a pH dependent 5-(E/Z)-isomer mixture.

(DMSO-d6, stored via K₂CO₃, primary isomer): δ=1.07 (t, 3H); 1.22 (t, 3H); 1.68 (m, 4H); 2.58 (m, 2H); 3.19 (pentuplet, 2H); 3.39 (m, 1H); 3.90 (m, 1H); 4.21 (q, 2H); 6.90 (s, 1H); 7.12-7.31 (m, 2H); 7.50-7.80 (m, 2H); 8.04 (s, 1H); 9.81-9.99 (s, b, 1H); 10.39 (s, 1H) ppm.

### Example 3

### 2-Cyano-N-ethyl-2-[3-ethyl-5-[1-{3-[3-(4-hydroxymethyl-piperidin-1-yl)-propionylamino]-phenylamino}-meth-(E/Z)-ylidene]-4-oxo-thiazolidin-(2-(E or Z))-ylidene]-acetamide

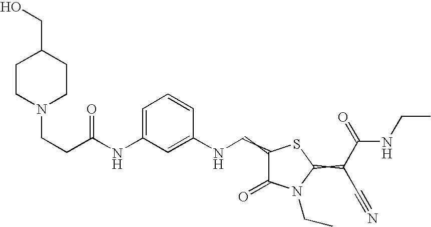

Dissolve 150 mg of the compound described under Example 19) in 5 ml tetrahydrofurane. Add 0.25 ml triethylamine and 62 mg Piperidin-4-yl-methanol. Stir for 12 hours under re-flow. Add a semi-saturated sodium hydrogencarbonate solution to the reaction mixture and extract with dichlormethane. Wash the organic solution with saturated sodium chloride, dry over sodium sulfate, press and, after purification through chromatography in silica gel, 37 mg of the compound in the title is obtained as a pH dependent 5-(E/Z)-isomer mixture.

(DMSO-d6, stored via K₂CO₃, primary isomer): δ=0.97-1.40 (m, 9H); 1.64 (d, 2H); 1.90 (t, 2H); 2.45 (m, 2H); 2.60 (t, 2H); 2.89 (m, 2H); 3.11-3.29 (m, 4H); 4.21 (q, 2H); 4.49 (t, 1H); 6.92 (s, 1H); 7.13 (d, 1H); 7.24 (t, 1H); 7.56-7.80 (m, 2H); 8.02 (s, 1H); 10.18 (s, 1H); 10.40 (s, 1H) ppm.

### Example 4

### 2-Cyano-2-[3-ethyl-5-[1-(3-hydroxymethyl-phenylamino)-meth-(E/Z)-ylidene]-4-oxo-thiazolidin-(2-(E or Z))-ylidene]-N-prop-2-ynyl-acetamide

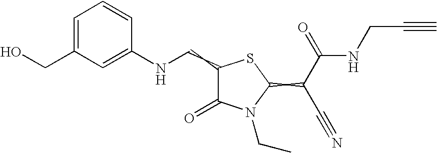

Dissolve 50 mg of the compound described under Intermediate INT9) in 5 ml triethylorthoformiate. Add 148 mg 3-aminobenzyl alcohol and 100 μl triethylorthoformiate. Stir for 3 hours under re-flow. Filter off the excluded product after the cooling of the reaction mixture. After purification through the re-crystallizing of ethanol, 56 mg of the compound in the title is obtained.

1H-NMR (DMSO-d6, stored over K₂CO₃, primary isomer): δ=1.24 (t, 3H); 3.07 (s, b, 1H); 3.92 (m, 2H); 4.23 (q, 2H); 4.49 (d, 2H); 5.25 (t, 1H); 7.00 (d, 1H); 7.13 (d, 1H); 7.21-7.35 (m, 2H); 7.95-8.20 (m, 2H); 10.40 (s, 1H) ppm.

### Example 5

### 2-Cyano-N-ethyl-2-[3-ethyl-4-oxo-5-[1-[3-(2-piperidin-1-yl-acetylamino)-phenylamino]-meth-(E/Z)-ylidene]-thiazolidin-(2-(E or Z))-ylidene]-acetamide

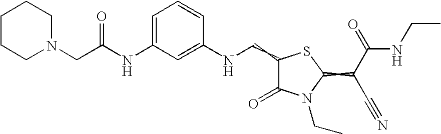

Dissolve 50 mg of the compound described under Intermediate INTT7) in 10 ml ethanol. Add 140 mg of the compound described under Intermediate INT20) and 100 μL triethylorthoformiate to it. Stir for 3 hours under re-flow. Press the reaction mixture. After purification through the re-crystallizing of ethanol, 26 mg of the compound in the title is obtained as a pH dependent 5-(E/Z)-isomer mixture.

1H-NMR (DMSO-d6, stored over K₂CO₃, primary isomer): δ=1.07 (t, 1H); 1.25 (t, 3H); 1.41 (m, 2H); 1.59 (m, 4H); 2.44 (m, 4H); 3.06 (s, 2H); 3.20 (pentuplet, 2H); 4.23 (q, 2H); 6.96 (d, 1H); 7.20-7.33 (m, 2H); 7.60-7.77 (m, 2H); 8.03 (s, 1H); 9.70 (s, 1H); 10.39 (s, 1H) ppm.

### Example 6

### 2-[5-[1-(3-Amino-phenylamino)-meth-(E/Z)-ylidene]-3-ethyl-4-oxo-thiazolidin-(2-(E or Z))-ylidene]-2-cyano-N-ethyl-acetamide

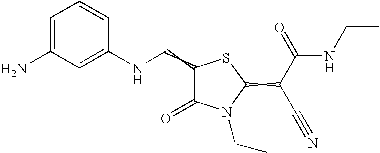

Suspend 7.75 g of the compound manufactured under Example 79) in 120 ml dichlormethane. Add 70 ml trifluoro acetic acid to it. Stir for one hour at room temperature. Press the reaction mixture, add dichlormethane and hexane and press anew. After drying well in vacuum, 11.2 g of the compound in the title is obtained as a trifluoro acetic acid salt. This raw product is used without further purification for the following reactions.

1H-NMR (DMSO-d6, stored over K₂CO₃, primary isomer): δ=1.07 (t, 3H); 1.26 (t, 3H); 3.20 (m, 2H); 4.22 (q, 2H); 6.80 (d, 1H); 7.01 (s, 1H); 7.05 (d, 1H); 7.30 (t, 1H); 7.74 (t, 1H); 8.01 (d, 1H); 9.20 (s, b, 3H); 10.35 (d, 1H) ppm.

### Example 7

### 2-[5-[1-[3-(2-Chloro-acetylamino)-phenylamino]-meth-(E/Z)-ylidene]-3-ethyl-4-oxo-thiazolidin-(2-(E or Z))-ylidene]-2-cyano-N-ethyl-acetamide

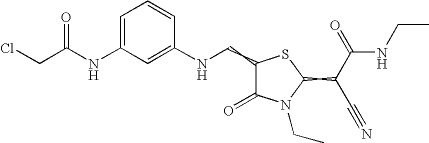

Suspend approx. 16.9 mmol of the raw product of the compound manufactured under Example 6) (11.2 g) in 500 ml tetrahydrofurane. Add 5.15 ml triethylamine at room temperature and 3.28 g chloro-acetic acid anhydride in portions following this at 15° C. Stir for two hours at room temperature. Add a semi-saturated sodium hydrogencarbonate solution to the reaction mixture and extract with acetic acid ethylester. Wash the organic solution with a saturated sodium chloride solution, dry over sodium sulfate, press and, after purification through re-crystallization from ethanol, 5.26 g of the compound in the title is obtained as a pH dependent 5-(E/Z)-isomer mixture.

1H-NMR (DMSO-d6, stored over K₂CO₃, primary isomer): δ=1.09 (t, 3H); 1.26 (t, 3H); 3.21 (pentuplet, 2H); 4.21 (q, 2H); 4.28 (s, 2H); 7.00 (d, 1H); 7.20 (d, 1H); 7.29 (t, 1H); 7.58-7.77 (m, 1H); 8.01 (s, 1H); 10.35 (s, 1H); 10.41 (s, 1H) ppm.

### Example 8

### 2-Cyano-N-ethyl-2-[3-ethyl-5-[1-{3-[2-(4-methyl-piperidin-1-yl)-acetylamino]-phenylamino}-meth-(E/Z)-ylidene]-4-oxo-thiazolidin-(2-(E or Z))-ylidene]-acetamide

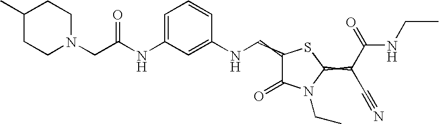

Dissolve 100 mg of the compound described under Example 7) in 5 ml dimethylformamide. Add 0.15 ml triethylamine, 6 mg potassium iodide and 38 μl 4-methylpiperidine to it. Stir for 4 hours at room temperature. Add a semi-saturated sodium hydrogencarbonate solution to the reaction mixture and extract with acetic acid ethylester. Wash the organic solution with saturated sodium chloride, dry over sodium sulfate, press and, after purification through chromatography in silica gel, 62 mg of the compound in the title is obtained as a pH dependent 5-(E/Z)-isomer mixture.

1H-NMR (DMSO-d6, stored over K₂CO₃, primary isomer): δ=0.91 (d, 3H); 1.08 (t, 3H); 1.14-1.40 (m, 6H); 1.59 (d, 2H); 2.12 (t, 2H); 2.83 (d, 2H); 3.09 (s, 2H); 3.21 (m, 2H); 4.22 (q, 4H); 6.96 (d, 2H); 7.20-7.33 (m, 2H); 7.58-7.78 (m, 2H); 8.04 (s, 1H); 9.69 (s, 1H); 10.40 (s, 1H) ppm.

### Example 9

### 2-[5-[1-{3-[2-(4-Acetyl-piperazin-1-yl)-acetylamino]-phenylamino}-meth-(E/Z)-ylidene]-3-ethyl-4-oxo-thiazolidin-(2-(E or Z))-ylidene]-2-cyano-N-ethyl-acetamide

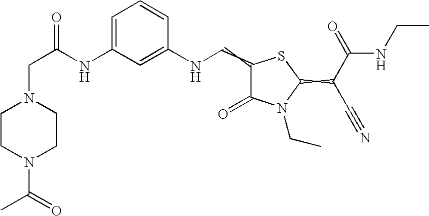

Suspend 94 mg of the compound manufactured under Example 80) in 5 ml dichlormethane. Add 2.5 ml trifluoro-acetic acid to it. Stir for 30 minutes at room temperature. Press the reaction mixture, add dichlormethane and hexane and press anew. After drying well in vacuum, the residue thus obtained is suspended in 5 ml dimethylformamide. Add 50 μL acetic acid, 67 mg sodium hydrogencarbonate and 62 mg TBTU 5A to it. Stir for 12 hours at room temperature. Add a semi-saturated sodium hydrogencarbonate solution to the reaction mixture and extract with acetic acid ethylester. Wash the organic solution with a saturated sodium chloride solution, dry over sodium sulfate, press and, after purification through re-crystallization from ethanol, 48 mg of the compound in the title is obtained as a pH dependent 5-(E/Z)-isomer mixture.

1H-NMR (DMSO-d6, stored over K₂CO₃, primary isomer): δ=1.07 (t, 3H); 1.25 (t, 3H); 2.00 (s, 3H); 2.41-2.60 (m, 4H); 3.14-3.28 (m, 4H); 3.50 (m, 4H); 4.22 (q, 2H); 6.98 (m, 1H); 7.21-7.31 (m, 2H); 7.63-7.76 (m, 2H); 8.00 (s, 1H); 9.81 (s, 1H); 10.40 (s, 1H) ppm.

### Example 10

### 2-Cyano-N-ethyl-2-[3-ethyl-5-[1-{3-[2-(4-methanesulfonyl-piperazin-1-yl)-acetylamino]-phenylamino}-meth-(E/Z)-ylidene]-4-oxo-thiazolidin-(2-(E or Z))-ylidene]-acetamide

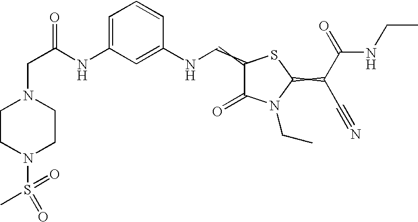

Suspend 120 mg of the compound manufactured under Example 80) in 5 ml dichlormethane. Add 2.5 ml trifluoro-acetic acid to it. Stir for 30 minutes at room temperature. Press the reaction mixture, add dichlormethane and hexane and press anew. After drying well in vacuum, the residue thus obtained is suspended in 5 ml tetrahydrofurane. Add 50 μL triethylamine, 20 μL methano-sulfonic acid chloride to it. Stir for 3 hours at room temperature. Add semi-saturated sodium hydrogencarbonate solution to the reaction mixture and extract with acetic acid ethylester. Wash the organic solution with a saturated sodium chloride solution, dry over sodium sulfate, press and, after purification through re-crystallization from ethanol, 46 mg of the compound in the title is obtained as a pH dependent 5-(E/Z)-isomer mixture.

1H-NMR (DMSO-d6, stored over K₂CO₃, primary isomer): δ=1.08 (t, 3H); 1.24 (t, 3H); 2.63 (m, 4H); 2.91 (s, 3H); 3.10-3.28 (m, 8H); 4.22 (q, 2H); 6.95 (s, 1H); 7.20-7.30 (m, 2H); 7.56-7.75 (m, 2H); 8.05 (s, 1H); 9.80 (s, 1H); 10.40 (s, 1H) ppm.

### Example 11

### 2-Cyano-N-cyanomethyl-2-[3-ethyl-5-[1-[3-(2-hydroxy-acetylamino)-phenylamino]-meth-(E/Z)-ylidene]-4-oxo-thiazolidin-(2-(E or Z))-ylidene]-acetamide

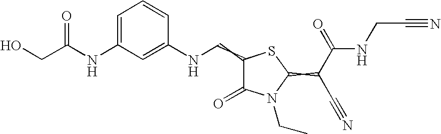

Dissolve 100 mg of the compound manufactured under Example 95) in 10 ml methanol. Add 1 ml water and 30 mg potassium carbonate to it. Stir for 2 hours at room temperature. Add water to the reaction mixture and extract with acetic acid ethylester. Wash the organic solution with a saturated sodium chloride solution, dry over sodium sulfate, press and, after purification through re-crystallization from ethanol, 72 mg of the compound in the title is obtained as a pH dependent 5-(E/Z)-isomer mixture.

1H-NMR (DMSO-d6, stored over K₂CO₃, primary isomer): δ=1.26 (t, 3H); 4.01 (d, 1H); 4.17 (d, 2H); 4.25 (q, 2H); 5.70 (t, 1H); 6.99 (d, 2H); 7.28 (t, 1H); 7.40 (d, 1H); 7.81 (s, 1H); 8.09 (s, 1H); 8.35 (s, 1H); 9.73 (s, 1H); 10.53 (s, 1H) ppm.

### Example 12

### Methanesulfonic acid 2-(3-{[2-[1-cyano-1-(cyanomethyl-carbamoyl)-meth-(E or Z)-ylidene]-3-ethyl-4-oxo-thiazolidin-(5-(E/Z))-ylidenemethyl]-amino}-phenyl)-ethyl ester

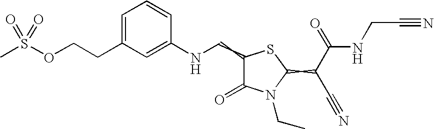

Dissolve 1.0 g of the compound manufactured under Example 71) in 10 ml dimethylformamide and 200 ml tetrahydrofurane. Add 0.9 ml triethylamine and 0.31 ml methane sulfonic acid chloride to it at −10° C. Stir for 1 hour at room temperature. Add a semi-saturated sodium hydrogencarbonate solution to the reaction mixture and extract with acetic acid ethylester. Wash the organic solution with a saturated sodium chloride solution, dry over sodium sulfate and press. Add dichlormethane to the solid obtained, stir for one hour at room temperature and filter off. 1.0 g of the compound in the title is obtained as a pH dependent 5-(E/Z)-isomer mixture.

1H-NMR (DMSO-d6, stored over K₂CO₃, primary isomer): δ=1.26 (t, 3H); 3.00 (t, 2H); 3.11 (s, 3H); 4.17 (m, 2H); 4.24 (q, 2H); 4.45 (t, 2H); 7.01 (d, 1H); 7.19 (d, 1H); 7.25-7.36 (m, 2H); 8.19 (s, 1H); 8.34 (t, 1H); 10.41 (s, 1H) ppm.

### Example 13

### 2-Cyano-N-cyanomethyl-2-[3-ethyl-5-[1-[3-(2-iodo-ethyl)-phenylamino]-meth-(E/Z)-ylidene]-4-oxo-thiazolidin-(2-(E or Z))-ylidene]-acetamide

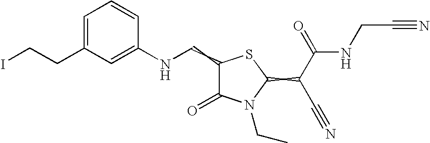

Dissolve 4.5 g of the compound manufactured under Example 12) in 400 ml butanon. Add 1.72 g sodium iodide to it. Stir for 8 hours under re-flow. Add water to the reaction mixture and extract with acetic acid ethylester. 1.6 g of the initial material is re-obtained from the watery phase through filtration. Dry the organic solution over sodium sulfate and press. 3.0 g of the compound in the title is obtained as a pH dependent 5-(E/Z)-isomer mixture.

1H-NMR (DMSO-d6, stored over K₂CO₃, primary isomer): δ=1.27 (t, 3H); 3.12 (t, 2H); 3.50 (t, 2H); 4.16 (d, 2H); 4.24 (q, 2H); 6.98 (d, 1H); 7.18 (d, 1H); 7.22-7.34 (m, 2H); 8.20 (d, 1H); 8.35 (t, 1H); 10.41 (d, 1H) ppm.

### Example 14

### 2-Cyano-N-cyanomethyl-2-[3-ethyl-5-[1-[3-(2-morpholin-4-yl-ethyl)-phenylamino]-meth-(E/Z)-ylidene]-4-oxo-thiazolidin-(2-(E or Z))-ylidene]-acetamide

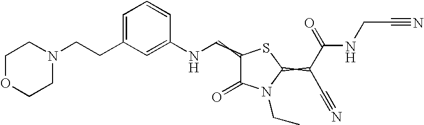

Dissolve 120 mg of the compound manufactured under Example 13) in 5 ml dimethylformamide. Add 42 mg morpholine and 65 mg potassium carbonate to it. Stir for 12 hours at room temperature. Add water to the reaction mixture and extract with acetic acid ethylester. Wash the organic solution with saturated sodium chloride, dry over sodium sulfate, press and, after purification through chromatography in silica gel, 40 mg of the compound in the title is obtained as a pH dependent 5-(E/Z)-isomer mixture.

1H-NMR (DMSO-d6, stored over K₂CO₃, primary isomer): δ=1.27 (t, 3H); 2.43 (m, 4H); 2.52 (m, 2H); 2.74 (m, 2H); 3.59 (m, 4H); 4.17 (m, 2H); 4.23 (q, 2H); 6.95 (d, 1H); 7.11 (d, 1H); 7.19-7.30 (m, 2H); 8.18 (s, 1H); 8.32 (s, 1H); 10.39 (s, 1H) ppm.

The following compounds are manufactured according to the process described above.

| | | | Molecular | |
|---|---|---|---|---|
| | | | Weight/ | |
| | | | MS | Educt/ |
| Example | | | (ESI) | Additional |
| no. | Structure and name | ¹H-NMR | [M + 1]⁺ | synthesis |
| 15 | 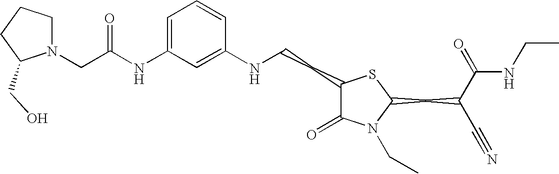 2-Cyano-N-ethyl-2-[3-ethyl-5-[1-{3- [2-((S)-2-hydroxymethyl-pyrrolidin- 1-yl)-acetylamino]-phenylamino}- meth-(E/Z)-ylidene]-4-oxo- thiazolidin-(2-(E or Z))-ylidene]- acetamide | (DMSO-d6, stored via K₂CO₃, primary isomer): δ = 1.06 (t, 3H); 1.23 (t, 3H); 1.51-1.91 (m, 4H); 2.44 (m, 1H); 2.62-2.75 (m, 1H); 3.01-3.77 (m, 4H); 3.38 (m, 2H); 3.54 (d, 1H); 4.22 (q, 2H); 4.68 (t, 1H); 6.97 (s, 1H); 7.20-7.32 (m, 2H); 7.56-7.78 (m, 2H); 8.04 (s, 1H); 9.81 (s, 1H); 10.40 (s, 1H) ppm. | 498.61/ 499 | 7/8 |
| 16 | 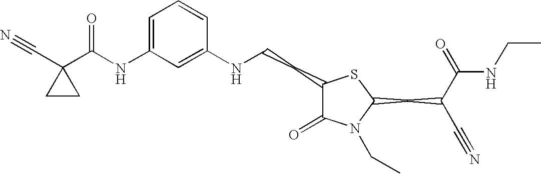 1-Cyano-cyclopropanecarboxylic acid (3-{[2-[1-cyano-1- ethylcarbamoyl-meth-(E or Z)- ylidene]-3-ethyl-4-oxo-thiazolidin- (5-(E/Z))-ylidenemethyl]-amino}- phenyl)-amide | (DMSO-d6, stored via K₂CO₃, primary isomer): δ = 1.08 (t, 3H); 1.23 (t, 3H); 1.69 (s, 4H); 3.20 (pentuplet, 2H); 4.21 (q, 2H); 7.00 (s, 1H); 7.21-7.33 (m, 2H); 7.52-7.77 (m, 2H); 8.02 (s, 1H); 10.03 (s, 1H); 10.38 (s, 1H) ppm. | 450.52/451 | 6/2 |
| 17 | 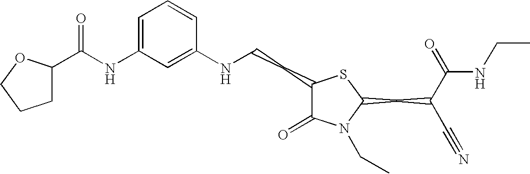 Tetrahydro-furan-2-carboxylic acid (3-{[2-[1-cyano-1-ethylcarbamoyl- meth-(E or Z)-ylidene]-3-ethyl-4- oxo-thiazolidin-(5-(E/Z))- ylidenemethyl]-amino}-phenyl)- amide | (CDCl₃, stored via K₂CO₃, primary isomer): δ = 1.21 (t, 3H); 1.39 (t, 3H); 1.85-1.97 (m, 4H); 3.30-3.48 (m, 2H); 3.89-4.11 (m, 2H); 4.37 (m, 2H); 4.48 (m, 1H); 6.19 (m, 1H); 6.80 (d, 1H); 7.05 (d, 1H); 7.25-7.42 (m, 1H); 7.58 (d, 1H); 7.70 (s, 1H); 8.56 (s, 1H); 10.49 (d, 1H) ppm. | 455.54/ 456 | 6/2 |
| 18 | 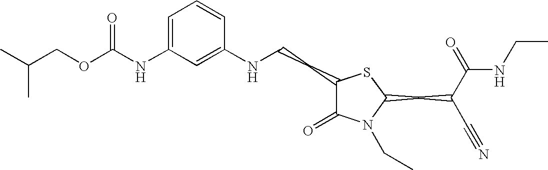 (3-{[2-[1-Cyano-1-ethylcarbamoyl- meth-(E or Z)-ylidene]-3-ethyl-4- oxo-thiazolidin-(5-(E/Z))- ylidenemethyl]-amino}-phenyl)- carbamic acid isobutyl ester | (DMSO-d6, stored via K₂CO₃, primary isomer): δ = 0.93 (d,6H); 1.08 (t, 3H); 1.24 (t, 3H); 1.93 (m, 1H); 3.20 (pentuplet, 2H); 3.89 (d, 2H); 4.22 (q, 2H); 7.39 (s, 1H); 7.09 (d, 1H); 7.21 (t, 1H); 7.49 (s, 1H); 7.68 (s, 1H); 8.00 (s, 1H); 9.68 (s, 1H); 10.40 (s, 1H) ppm. | 457.55/ 458 | 6/7 |
| 19 | 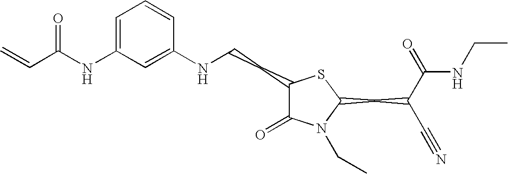 N-(3-{[2-[1-Cyano-1- ethylcarbamoyl-meth-(E or Z)- ylidene]-3-ethyl-4-oxo-thiazolidin- (5-(E/Z))-ylidenemethyl]-amino}- phenyl)-acrylamide | (DMSO-d6, stored via K₂CO₃, primary isomer): δ = 1.08 (t, 3H); 1.24 (t, 3H); 3.20 (pentuplet, 2H); 4.21 (q, 2H); 5.28 (m, 1H); 6.27 (m, 1H); 6.44 (m, 1H); 6.91-7.04 (m, 1H); 7.21-7.32 (m, 2H); 7.69 (m, 1H); 7.77 (s, 1H); 8.00 (s, 1H); 10.22 (s, 1H)ppm. | 411.48/ 412 | 6/2 |
| 20 | 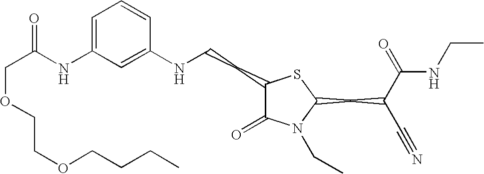 2-[5-[1-{3-[2-(2-Butoxy-ethoxy)- acetylamino]-phenylamino}-meth- (E/Z)-ylidene]-3-ethyl-4-oxo- thiazolidin-(2-(E or Z))-ylidene]-2- cyano-N-ethyl-acetamide | (DMSO-d6, stored via K₂CO₃, primary isomer): δ = 0.88 (t, 3H); 1.08 (t, 3H); 1.18-1.38 (m, 5H); 1.49 (pentuplet, 2H); 3.21 (pentuplet, 2H); 3.42 (t, 2H); 3.57 (t, 2H); 3.68 (t, 2H); 4.10 (s, 2H); 4.23 (q, 2H); 6.99 (m, 1H); 7.21-7.32(m, 2H); 7.64-7.78 (m, 2H); 8.01 (d, 1H); 9.69 (s, 1H); 10.40 (d, 1H) ppm. | 515.63/ 516 | 6/2 |
| 21 | 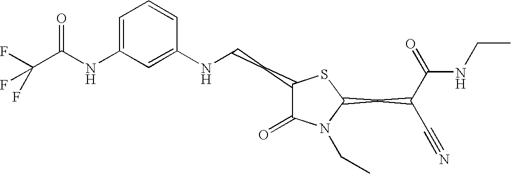 2-Cyano-N-ethyl-2-[3-ethyl-4-oxo- 5-[1-[3-(2,2,2-trifluoro- acetylamino)-phenylamino]-meth- (E/Z)-ylidene]-thiazolidin-(2-(E or Z))-ylidene]-acetamide | (DMSO-d6, stored via K₂CO₃, primary isomer): δ = 1.08 (t, 3H); 1.25 (t, 3H); 3.21 (pentuplet, 2H); 4.22 (q, 2H); 7.04 (s, 1H); 7.23-7.35 (m, 2H); 7.53-7.67 (m, 2H); 8.05 (s, 1H); 10.30-11.20 (b, 2H) ppm. | 453.44/ 454 | 6/2 |
| 22 | 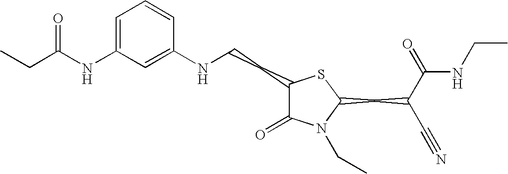 2-Cyano-N-ethyl-2-[3-ethyl-4-oxo- 5-[1-(3-propionylamino- phenylamino)-meth-(E/Z)-ylidene]- thiazolidin-(2-(E or Z))-ylidene]- acetamide | (DMSO-d6, stored via K₂CO₃, primary isomer): δ = 1.00-1.14 (m, 6H); 1.26 (t, 3H); 3.32 (q, 2H); 3.21 (pentuplet, 2H); 4.22 (q, 2H); 6.92 (d, 1H); 7.14-7.29 (m, 2H); 7.61-7.74 (m, 2H); 7.99 (s, 1H); 9.92 (s, 1H); 10.40 (s, 1H) ppm. | 413.50/ 414 | 6/2 |
| 23 | 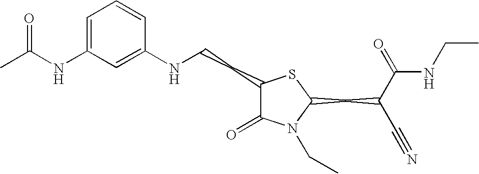 2[5-[1-(3-Acetylamino- phenylamino)-meth-(E/Z)-ylidene]- 2-ethyl-4-oxo-thiazolidin-(2-(E or Z))-ylidene]-2-cyano-N-ethyl- acetamide | (DMSO-d6, stored via K₂CO₃, primary isomer): δ = 1.09 (t, 3H); 1.24 (t, 3H); 2.05 (s, 3H); 3.21 (pentuplet, 2H); 4.22 (q, 2H); 6.94 (d, 1H); 7.16 (d, 1H); 7.24 (t, 1H); 7.60-7.76 (m, 2H); 7.99 (s, 1H); 10.00 (s, 1H); 10.40 (s, 1H) ppm. | 399.47/ 400 | 6/2 |
| 24 | 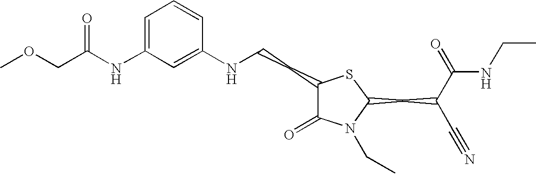 2-Cyano-N-ethyl-2-[3-ethyl-5-[1-[3- (2-methoxy-acetylamino)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.09 (t, 3H); 1.26 (t, 3H); 3.21 (pentuplet, 2H); 3.39 (s, 3H); 4.02 (s, 2H); 4.22 (q, 2H); 6.98 (d, 1H); 7.26 (t, 1H); 7.34 (d, 1H); 7.71 (t, 1H); 7.76 (s, 1H); 8.00 (d, 1H); 9.82 (s, 1H); 10.40(d, 1H) ppm. | 429.50/ 430 | 6/2 |
| 25 | 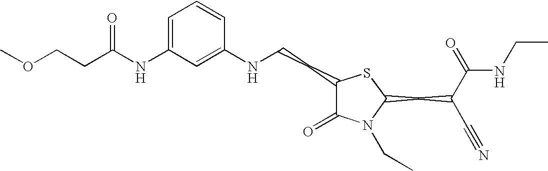 2-Cyano-N-ethyl-2-[3-ethyl-5-[1-[3- (3-methoxy-propionylamino)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.08 (t, 3H); 1.25 (t, 3H); 2.55 (t, 2H); 3.20 (pentuplet, 2H); 3.25 (s, 3H); 3.62 (t, 2H); 4.22 (q, 2H); 6.93 (d, 1H); 7.19 (d, 1H); 7.24 (t, 1H); 7.58-7.79 (m, 2H); 8.00 (s, 1H); 10.00 (s,1H); 10.38 (s, 1H) ppm. | 443.53/ 444 | 6/2 |
| 26 | 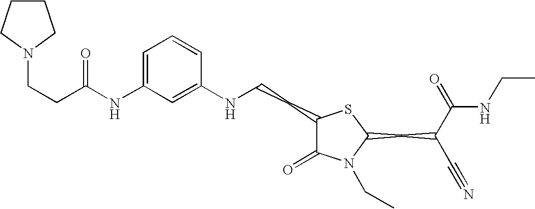 2-Cyano-N-ethyl-2-[3-ethyl-4-oxo- 5-[1-[3-(3-pyrrolidin-1-yl- propionylamino)-phenylamino]- meth-(E/Z)-ylidene]-thiazolidin-(2- (E or Z))-ylidene]-acetamide | (DMSO-d6, stored via K₂CO₃, primary isomer): δ = 1.08 (t, 3H); 1.24 (t, 3H); 1.70 (m, 4H); 2.39-2.60 (m, 6H); 2.73 (t, 2H); 3.20 (pentuplet, 2H); 4.23 (q, 2H); 6.96 (d, 1H); 7.16 (d, 1H); 7.25 (t, 1H); 7.65-7.77 (m, 2H); 7.99 (d, 1H); 10.14 (s, 1H); 10.39 (d, 1H) ppm. | 482.60/ 483 | 19/3 |
| 27 | 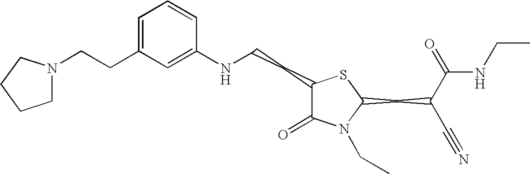 2-Cyano-N-ethyl-2-[3-ethyl-4-oxo- 5-[1-[3-(2-pyrrolidin-1-yl-ethyl)- phenylamino]-meth-(E/Z)-ylidene]- thiazolidin-(2-(E or Z))-ylidene]- acetamide | (DMSO-d6, stored via K₂CO₃, primary isomer): δ = 1.08 (t, 3H); 1.27 (t, 3H); 1.68 (m, 4H); 2.47 (m, 4H); 2.64 (m, 2H); 2.72 (m, 2H); 3.20 (pent7uplet, 2H); 4.22 (q, 2H); 6.92 (d, 1H); 7.10 (d, 1H); 7.16-7.28 (m, 2H); 7.70 (t, 1H); 8.09 (s,1H); 10.24 (s, 1H) ppm. | 439.58/ 440 | INTA1/1 |
| 28 | 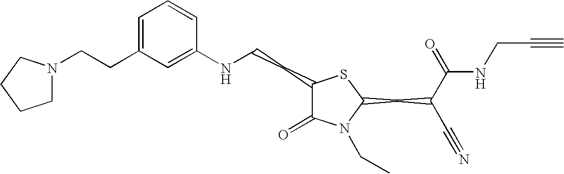 2-Cyano-2-[3-ethyl-4-oxo-5-[1-[3- (2-pyrrolidin-1-yl-ethyl)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.25 (t, 3H); 1.68 (m, 4H); 2.48 (m, 4H); 2.62 (m, 2H); 2.73 (m, 2H); 3.06 (s,b, 1H); 3.93 (m, 2H); 4.23 (q, 2H); 6.93 (d, 1H); 7.10 (d, 1H); 7.16-7.30 (m, 2H); 8.08 (t, 1H); 8.12 (s, 1H); 10.30 (s, 1H) ppm. | 449.58/ 450 | INTA1/ 1 |
| 29 | 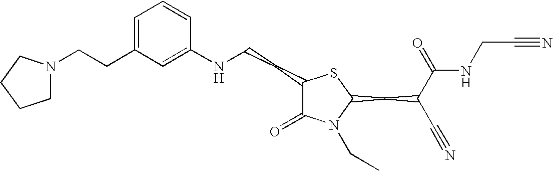 2-Cyano-N-cyanomethyl-2-[3-ethyl- 4-oxo-5-[1-[3-(2-pyrrolidin-1-yl- ethyl)-phenylamino]-meth-(E/Z)- ylidene]-thiazolidin-(2-(E or Z))- ylidene]-acetamide | (DMSO-d6, stored via K₂CO₃, primary isomer): δ = 1.25 (t, 3H); 1.68 (m, 4H); 2.50 (m, 4H); 2.64 (m, 2H); 2.73 (m, 2H); 4.17 (d, 2H); 4.23 (q, 2H); 6.94 (d, 1H); 7.10 (d, 1H); 7.17-7.31 (m, 2H); 8.16 (s, 1H); 8.35 (s, 1H); 10.38 (s, 1H) ppm. | 450.56/ 451 | INTA1/ 1 |
| 30 | 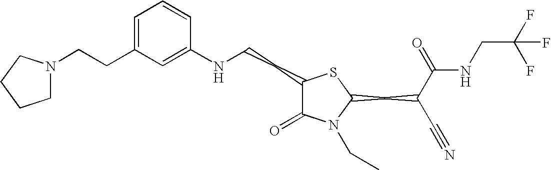 2-Cyano-2-[3-ethyl-4-oxo-5-[1-[3- (2-pyrrolidin-1-yl-ethyl)- phenylamino]-meth-(E/Z)-ylidene]- thiazolidin-(2-(E or Z))-ylidene]-N- (2,2,2-trifluoro-ethyl)-acetamide | (DMSO-d6, stored via K₂CO₃, primary isomer): δ = 1.26 (t, 3H); 1.70 (m, 4H); 2.47 (m, 4H); 2.63 (m, 2H); 2.74 (m, 2H); 3.97 (m, 2H); 4.25 (q, 2H); 6.95 (d, 1H); 7.12 (d, 1H); 7.19-7.30 (m, 2H); 8.15 (s, 1H); 8.21 (t, 1H); 10.38 (s,1H ppm. | 493.55/ 494 | INTA1/ 1 |
| 31 | 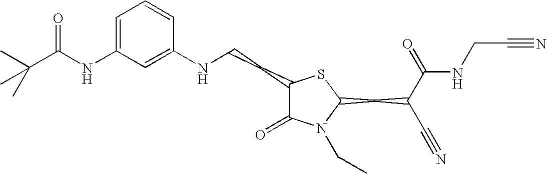 2-Cyano-N-cyanomethyl-2-[5-[1-[3- (2,2-dimethyl-propionylamino)- phenylamino]-meth-(E/Z)-ylidene]- 3-ethyl-4-oxo-thiazolidin-(2-(E or Z))-ylidene]-acetamide | (DMSO-d6, stored via K₂CO₃, primary isomer): δ = 1.19-1.30 (m, 12H); 4.16 (d, 2H); 4.24 (q, 2H); 6.95 (d, 1H); 7.24 (t, 1H); 7.37 (d, 1H); 7.72 (s, 1H); 8.09 (s, 1H); 8.32 (s, 1H); 9.25 (s, 1H); 10.53 (s, 1H) ppm. | 452.54/ 453 | INTA4/ 1 |
| 32 | 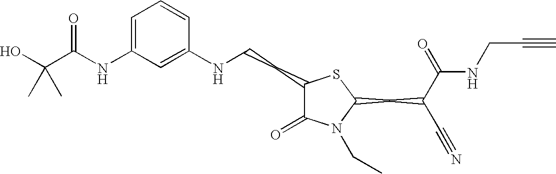 2-Cyano-2-[3-ethyl-5-[1-[3-(2- hydroxy-2-methyl-propionylamino)- phenylamino]-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 1.26 (t, 3H); 1.38 (s, 6H); 3.08 (s, b, 1H); 3.93 (m, 2H); 4.24 (q, 2H); 5.76 (s, 1H); 6.97 (d, 1H); 7.25 (t, 1H); 7.43 (d, 1H); 7.87 (s, 1H); 8.00-8.16 (m, 2H); 9.65 (s, 1H); 10.42(d, 1H) ppm. | 453.52/ 454 | INTA10/ 1 |
| 33 | 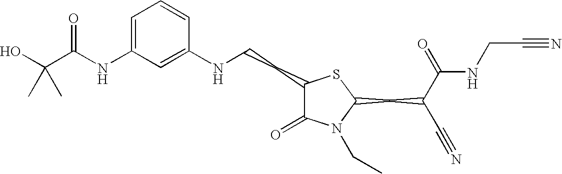 2-Cyano-N-cyanomethyl-2-[3-ethyl- 5-[1-[3-(2-hydroxy-2-methyl- propionylamino)-phenylamino]- meth-(E/Z)-ylidene]-4-oxo- thiazolidin-(2-(E or Z))-ylidene]- acetamide | (DMSO-d6, stored via K₂CO₃, primary isomer): δ = 1.27 (t, 3H); 1.37 (s, 6H); 4.17 (d, 2H); 4.25 (q, 2H); 5.76 (s, 1H); 6.98 (d, 1H); 7.26 (t, 1H); 7.45 (d, 1H); 7.38 (s, 1H); 8.09 (d, 1H); 8.34 (t, 1H); 9.66 (s, 1H); 10.50 (d, 1H)ppm. | 454.51/ 455 | INTA10/ 1 |
| 34 | 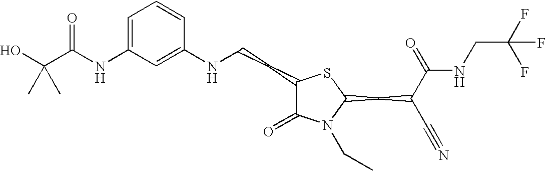 2-Cyano-2-[3-ethyl-5-[1-[3-(2- hydroxy-2-methyl-propionylamino)- phenylamino]-meth-(E/Z)-ylidene]- 4-oxo-thiazolidin-(2-(E or Z))- ylidene]-N-(2,2,2-trifluoro-ethyl)- acetamide | (DMSO-d6, stored via K₂CO₃, primary isomer): δ = 1.27 (t, 3H); 1.35 (s, 6H); 3.95 (m, 2H); 4.25 (q, 2H); 5.75 (s, 1H); 6.08 (d, 1H); 7.25 (t, 1H); 7.43 (d, 1H); 7.88 (s, 1H); 8.09 (d, 1H); 8.21 (t, 1H); 9.65 (s, 1H); 10.48 (d, 1H) ppm. | 497.50/ 498 | INTA10/ 1 |
| 35 | 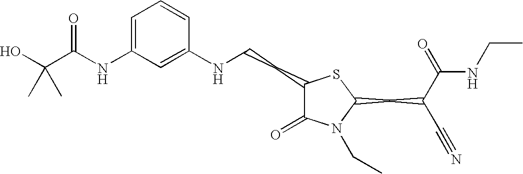 2-Cyano-N-ethyl-2-[3-ethyl-5-[1-[3- (2-hydroxy-2-methyl- propionylamino)-phenylamino]- meth-(E/Z)-ylidene]-4-oxo- thiazolidin-(2-(E or Z))-ylidene]- acetamide | (DMSO-d6, stored via K₂CO₃, primary isomer): δ = 1.18 (t, 3H); 1.25 (t, 3H); 1.35 (s, 6H); 3.21 (pentuplet, 2H); 4.24 (q, 2H); 5.75 (s, 1H); 6.96 (d, 1H); 7.24 (t, 1H); 7.42 (d, 1H); 7.70 (t, 1H); 7.85 (s, 1H); 8.03 (d, 1H); 9.64(s, 1H); 10.36 (d, 1H) ppm. | 443.53/ 444 | INTA10/ 1 |
| 36 | 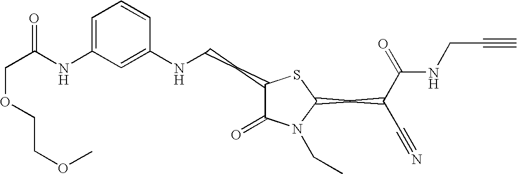 2-Cyano-2-[3-ethyl-5-[1-{3-[2-(2- methoxy-ethoxy)-acetylamino]- phenylamino}-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 1.24 (t, 3H); 3.06 (m, 1H); 3.31 (s, 3H); 3.54 (m, 2H); 3.68 (m, 2H); 3.93 (m, 2H); 4.10 (s, 2H); 4.23 (q, 2H); 7.01 (m, 1H); 7.23-7.34 (m, 2H); 7.75 (s, 1H); 8.03 (d, 1H); 8.10 (t,1H); 9.70 (s, 1H); 10.45 (d, 1H) ppm. | 483.55/ 484 | INTA5/nl 1 |
| 37 | 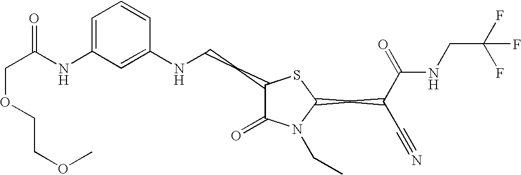 2-Cyano-2-[3-ethyl-5-[1-{3-[22-(2- methoxy-ethoxy)-acetylamino]- phenylamino}-meth-(E/Z)-ylidene]- 4-oxo-thiazolidin-(2-(E or Z))- ylidene]-N-(2,2,2-trifluoro-ethyl)- acetamide | (DMSO-d6, stored via K₂CO₃, primary isomer): δ = 1.26 (t, 3H); 3.31 (s, 3H); 3.54 (m, 2H); 3.69 (m, 2H); 3.97 (m, 2H); 4.10 (s, 2H); 4.25 (q, 2H); 7.01 (m, 1H); 7.22-7.34 (m, 2H); 7.76 (s, 1H); 8.07 (d, 1H); 8.23(t, 1H); 9.71 (s, 1H); 10.51 (d, 1H) ppm. | 527.52/ 528 | INTA5/ 1 |
| 38 | 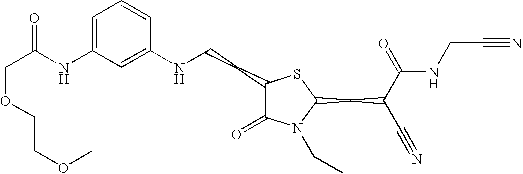 2-Cyano-N-cyanomethyl-2-[3-ethyl- 5-[1-{3-[2-(2-methoxy-ethoxy)- acetylamino]-phenylamino}-meth- (E/Z)-ylidene]-4-oxo-thiazolidin-(2- (E or Z))-ylidene]-acetamide | (DMSO-d6, stored via K₂CO₃, primary isomer): δ = 1.26 (t, 3H); 3.31 (s, 3H); 3.54 (m, 2H); 3.68 (m, 2H); 4.10 (s, 2H); 4.18 (d, 2H); 4.25 (q, 2H); 7.02 (m, 1H); 7.23-7.35 (m, 2H); 7.75 (s, 1H); 8.08 (d, 1H); 8.35 (t, 1H); 9.71(s, 1H); 10.55 (d, 1H) ppm. | 484.54/ 485 | INTA5/ 1 |
| 39 | 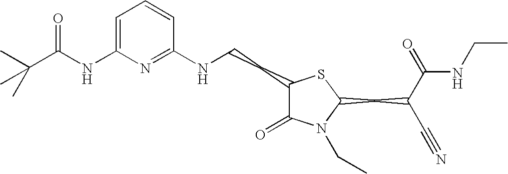 2-Cyano-2-[5-[1-[6-(2,2-dimethyl- propionylamino)-pyridin-2-ylamino]- meth-(E/Z)-ylidene]-3-ethyl-4-oxo- thiazolidin-(2-(E or Z))-ylidene]-N- ethyl-acetamide | (DMSO-d6, stored via K₂CO₃, primary isomer): δ = 1.09 (t, 3H); 1.18-1.30 (m, 12H); 3.21 (m, 2H); 4.23 (q, 2H); 6.78 (dd, 1H); 7.63-7.79 (m, 3H); 8.74 (s, 1H); 9.68 (s, 1H); 10.67 (s, 1H) ppm. | 442.54/ 443 | INTA3/ 1 |
| 40 | 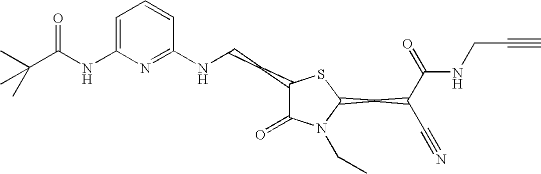 2-Cyano-2-[5-[1-[6-(2,2-dimethyl- propionylamino)-pyridin-2-ylamino]- meth-(E/Z)-ylidene]-3-ethyl-4-oxo- thiazolidin-(2-(E or Z))-ylidene]-N- prop-2-ynyl-acetamide | (DMSO-d6, stored via K₂CO₃, primary isomer): δ = 1.19-1.31 (m, 12H); 3.08 (m, 1H); 3.92 (m, 2H); 4.22 (q, 2H); 6.78 (dd, 1H); 7.65-7.76 (m, 2H); 8.14 (s, 1H); 8.78 (s, 1H); 9.68 (s, 1H); 10.75 (s, 1H) ppm. | 452.54/ 453 | INTA3/ 1 |
| 41 | 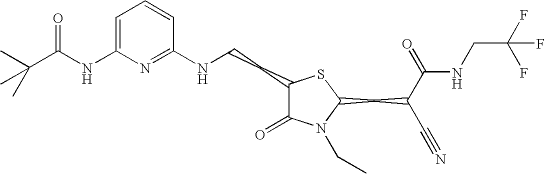 2-Cyano-2-[5-[1-[6-(2,2-dimethyl- propionylamino)-pyridin-2-ylamino]- meth-(E/Z)-ylidene]-3-ethyl-4-oxo- thiazolidin-(2-(E or Z))-ylidene]-N- (2,2,2-trifluoro-ethyl)-acetamide | (DMSO-d6, stored via K₂CO₃, primary isomer): δ = 1.15-1.31 (m, 12H); 3.95 (m, 2H); 4.22 (q, 2H); 6.71 (d, 1H); 7.58-7.72 (m, 2H); 8.02 (s, 1H); 8.88 (s, 1H); 9.55 (s, 1H); 10.80 (s, 1H) ppm. | 496.51/ 497 | INTA3/ 1 |
| 42 | 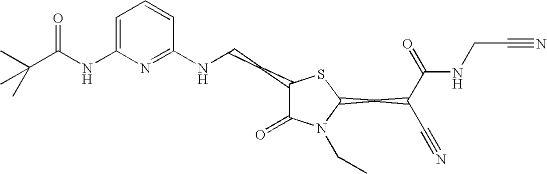 2-Cyano-N-cyanomethyl-2-[5-[1-[6- (2,2-dimethyl-propionylamino)- pyridin-2-ylamino]-meth-(E/Z)- ylidene]-3-ethyl-4-oxo-thiazolidin- (2-(E or Z))-ylidene]-acetamide | (DMSO-d6, stored via K₂CO₃, primary isomer): δ = 1.19-1.32 (m, 12H); 4.18 (d, 2H); 4.25 (q, 2H); 6.80 (d, 1H); 7.65-7.78 (m, 2H); 8.40 (t, 1H); 8.80 (s, 1H); 9.70 (s, 1H); 10.81 (s, 1H) ppm. | 453.52/ 454 | INTA3/ 1 |
| 43 | 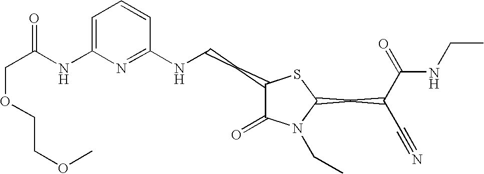 2-Cyano-N-ethyl-2-[3-ethyl-5-[1-{6- [2-(2-methoxy-ethoxy)- acetylamino]-pyridin-2-ylamino}- meth-(E/Z)-ylidene]-4-oxo- thiazolidin-(2-(E or Z))-ylidene]- acetamide | (DMSO-d6, stored via K₂CO₃, primary isomer): δ = 1.08 (t, 3H); 1.25 (t, 3H); 3.21 (m, 2H); 3.33 (s, 3H); 3.52 (m, 2H); 3.69 (m, 2H); 4.15 (s, 2H); 4.22 (q, 2H); 6.79 (dd, 1H); 7.64-7.81 (m, 3H); 8.67 (s, 1H); 9.94 (s, 1H); 10.75 (s, 1H) ppm. | 474.54/ 475 | INTA6/ 1 |
| 44 | 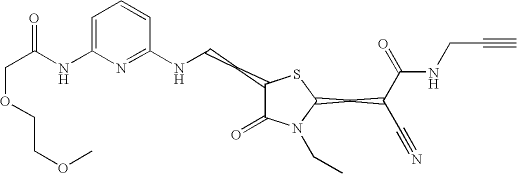 2-Cyano-2-[3-ethyl-5-[1-{6-[2-(2- methoxy-ethoxy)-acetylamino]- pyridin-2-ylamino}-meth-(E/Z)- ylidene]-4-oxo-thiazolidin-(2-(E or Z))-ylidene]-N-prop-2-ynyl- acetamide | (DMSO-d6, stored via K₂CO₃, primary isomer): δ = 1.24 (t, 3H); 3.09 (m, 1H); 3.35 (s, 3H); 3.51 (m, 2H); 3.69 (m, 2H); 3.92 (m, 2H); 4.15 (s, 2H); 4.22 (q, 2H); 6.82 (dd, 1H); 7.69-7.81 (m, 2H); 8.17 (t, 1H); 8.68 (s,1H); 9.99 (s, 1H); 10.85 (s, 1H) ppm. | 484.53/ 485 | INTA6/ 1 |
| 45 | 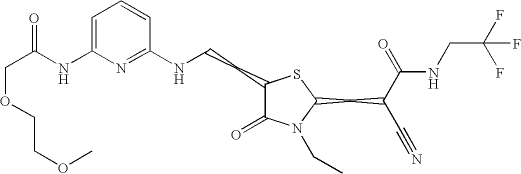 2-Cyano-2-[3-ethyl-5-[1-{6-[2-(2- methoxy-ethoxy)-acetylamino]- pyridin-2-ylamino}-meth-(E/Z)- ylidene]-4-oxo-thiazolidin-(2-(E or Z))-ylidene]-N-(2,2,2-trifluoro- ethyl)-acetamide | (DMSO-d6, stored via K₂CO₃, primary isomer): δ = 1.25 (t, 3H); 3.33 (s, 3H); 3.51 (m, 2H); 3.69 (m, 2H); 3.97 (m, 2H); 4.15 (s, 2H); 4.24 (q, 2H); 6.80 (dd, 1H); 7.68-7.82 (m, 2H); 8.28 (t, 1H); 8.70 (s, 1H); 9.99 (s, 1H); 10.87 (s, 1H) ppm. | 528.51/ 529 | INTA6/ 1 |
| 46 | 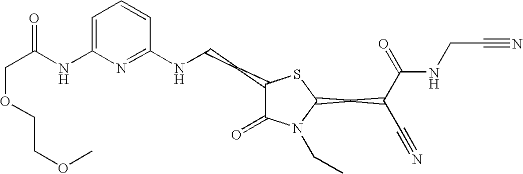 2-Cyano-N-cyanomethyl-2-[3-ethyl- 5-[1-{6-[2-(2-methoxy-ethoxy)- acetylamino]-pyridin-2-ylamino}- meth-(E/Z)-ylidene]-4-oxo- thiazolidin-(2-(E or Z))-ylidene]- acetamide | (DMSO-d6, stored via K₂CO₃, primary isomer): δ = 1.25 (t, 3H); 3.34 (s, 3H); 3.52 (m, 2H); 3.69 (m, 2H); 4.08-4.32 (m, 6H); 6.79 (d, 1H); 7.65-7.81 (m, 2H); 8.35 (s, 1H); 8.73 (s, 1H); 9.95 (s, 1H); 10.88 (s, 1H) ppm. | 485.52/ 486 | INTA6/ 1 |
| 47 | 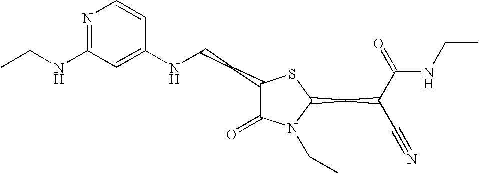 2-Cyano-N-ethyl-2-[3-ethyl-5-[1-(2- ethylamino-pyridin-4-ylamino)- meth-(E/Z)-ylidene]-4-oxo- thiazolidin-(2-(E or Z))-ylidene]- acetamide | (DMSO-d6, stored via K₂CO₃, primary isomer): δ = 0.99-1.17 (m, 6H); 1.25 (t, 3H); 3.12-3.29 (m, 4H); 4.21 (q, 2H); 6.22 (s, 1H); 6.38-6.50 (m, 2H); 7.75 (t, 1H); 7.83 (d, 1H); 7.99 (d, 1H); 10.20 (d, 1H) ppm. | 386.48/ 387 | INTA2/ 1 |
| 48 | 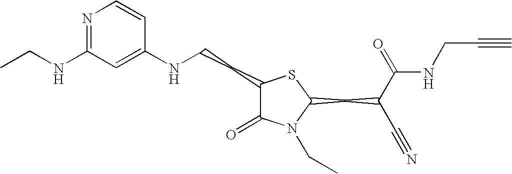 2-Cyano-2-[3-ethyl-5-[1-(2- ethylamino-pyridin-4-ylamino)- meth-(E/Z)-ylidene]-4-oxo- thiazolidin-(2-(E or Z))-ylidene-N- prop-2-ynyl-acetamide | (DMSO-d6, stored via K₂CO₃, primary isomer): δ = 1.11 (t, 3H); 1.26 (t, 3H); 3.07 (m, 1H); 3.23 (m, 2H); 3.92 (m, 2H); 4.22 (q, 2H); 6.28 (d, 1H); 6.44 (dd, 1H); 6.53 (t, 1H); 7.84 (d, 1H); 8.01 (d, 1H); 8.17 (t, 1H); 10.30 (d, 1H) ppm. | 396.47/ 397 | INTA2/ 1 |
| 49 | 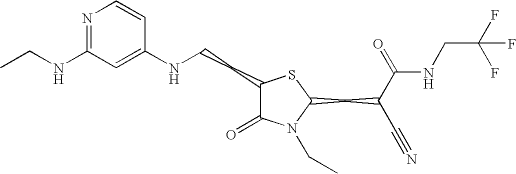 2-Cyano-2-[3-ethyl-5-[1-(2- ethylamino-pyridin-4-ylamino)- meth-(E/Z)-ylidene]-4-oxo- thiazolidin-(2-(E or Z))-ylidene]-N- (2,2,2trifluoro-ethyl)-acetamide | (DMSO-d₆, stored via K₂CO₃, primary isomer): δ = 1.11 (t, 3H); 1.26 (t, 3H); 3.22 (m, 2H); 3.97 (m, 2H); 4.24 (q, 2H); 6.24 (d, 1H); 6.40-6.50 (m, 2H); 7.84 (d, 1H); 8.03 (s, 1H); 8.27 (t, 1H); 10.31 (s, 1H) ppm. | 440.45/ 441 | INTA2/ 1 |
| 50 | 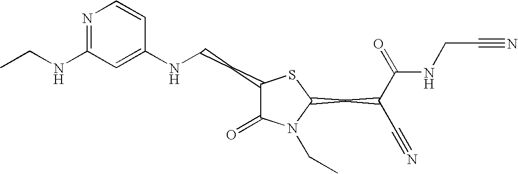 2-Cyano-N-cyanomethyl-2-[3-ethyl- 5-[1-(2-ethylamino-pyridin-4- ylamino)-meth-(E/Z)-ylidene]-4- ox-thiazolidin-(2-(E or Z))- ylidene]-acetamide | (DMSO-d6, stored via K₂CO₃, primary isomer): δ = 1.11 (t, 3H); 1.26 (t, 3H); 3.22 (m, 2H); 4.18 (d, 2H); 4.23 (q, 2H); 6.26 (d, 1H); 6.38-6.51 (m, 2H); 7.35 (d, 1H); 8.06 (d, 1H); 8.40 (t, 1H); 10.34 (d, 1H) ppm. | 397.46/ 398 | INTA2/ 1 |
| 51 | 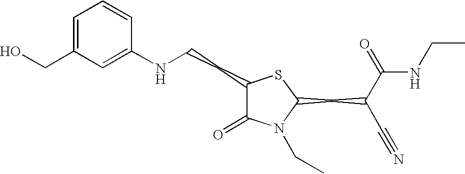 2-Cyano-N-ethyl-2-[3-ethyl-5-[1-(3- hydroxymethyl-phenylamino)-meth- (E/Z)-ylidene]-4-oxo-thiazolidin-(2- (E or Z))-ylidene]-acetamide | (DMSO-d6, stored via K₂CO₃, primary isomer): δ = 1.08 (t, 3H); 1.25 (t, 3H); 3.21 (pentuplet, 2H); 4.23 (q, 2H); 4.50 (d, 2H); 5.24 (t, 1H); 7.00 (d, 1H); 7.14 (d, 1H); 7.23-7.33 (m, 2H); 7.69 (t, 1H); 8.08 (s, 1H); 10.33 (s, 1H) ppm. | 372.45/ 373 | INTT7/ 4 |
| 52 | 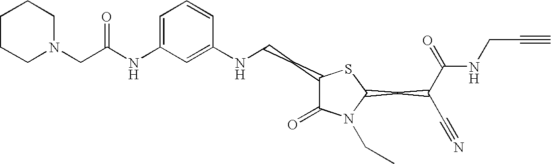 2-Cyano-2-[3-ethyl-4-oxo-5-[1-[3- (2-piperidin-1-yl-acetamino)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.26 (t, 3H); 1.41 (m, 2H); 1.59 (m, 4H); 2.45 (m, 4H); 3.01-3.11 (m, 3H); 3.92 (m, 2H); 4.24 (q, 2H); 7.00 (d, 1H); 7.21-7.35 (m, 2H); 7.72 (s, 1H); 8.00-8.15 (m, 2H); 9.71 (s, 1H); 10.43 (d,1H) ppm. | 492.60/ 493 | INTT9 + INT20/ 5 |
| 53 | 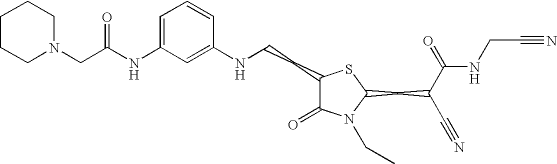 2-Cyano-N-cyanomethyl-2-[3-ethyl- 4-oxo-5-[1-[3-(2-piperidin-1-yl- acetylamino)-phenylamino]-meth- (E/Z)-ylidene]-thiazolidin-(2-(E or Z))-ylidene]-acetamide | (DMSO-d6, stored via K₂CO₃, primary isomer): δ = 1.27 (t, 3H); 1.42 (m, 2H); 1.58 (m, 4H); 2.47 (m, 4H); 3.08 (s, 2H); 4.17 (d, 2H); 4.24 (q, 2H); 6.94-7.05 (m, 1H); 7.21-7.34 (m, 2H); 7.75 (s, 1H); 8.09 (d, 1H); 8.36 (t, 1H); 9.74 (s,1H); 10.52 (d, 1H) ppm. | 493.59/ 494 | INTT10 + INT20/ 5 |
| 54 | 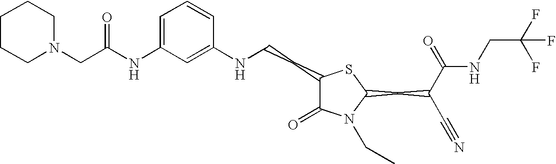 2-Cyano-2-[3-ethyl-4-oxo-5-[1-[3- (2-piperidin-1-yl-acetylamino)- phenylamino]-meth-(E/Z)-ylidene]- thiazolidin-(2-(E or Z))-ylidene]-N- (2,2,2-trifluoro-ethyl)-acetamide | (DMSO-d6, stored via K₂CO₃, primary isomer): δ = 1.27 (t, 3H); 1.41 (m, 2H); 1.59 (m, 4H); 2.45 (m, 4H); 3.08 (s, 2H); 3.97 (m, 2H); 4.24 (q, 2H); 7.00 (d, 1H); 7.21-7.34 (m, 2H); 7.74 (s, 1H); 8.08 (s, 1H); 8.21 (t, 1H); 9.72 (s, 1H); 10.50 (s, 1H) ppm. | 536.58/ 537 | INTT8 + INT20/ 5 |
| 55 | 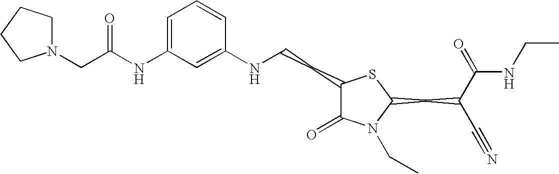 2-Cyano-N-ethyl-2-[3-ethyl-4-oxo- 5-[1-[3-(2-pyrrolidin-1-yl- acetylamino)-phenylamino]-meth- (E/Z)-ylidene]-thiazolidin-(2-(E or Z))-ylidene]-acetamide | (DMSO-d6, stored via K₂CO₃, primary isomer): δ = 1.08 (t, 3H); 1.25 (t, 3H); 1.77 (m, 4H); 2.60 (m, 4H); 3.21 (pentuplet, 2H); 3.26 (s, 2H); 4.23 (q, 2H); 6.97 (d, 2H); 7.20-7.37 (m, 2H); 7.62-7.78 (m, 2H); 8.02 (s, 1H); 9.76 (s, 1H); 10.39(s, 1H) ppm. | 468.58/ 469 | INTT7 + INT22/ 5 |
| 56 | 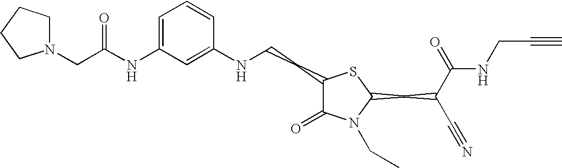 2-Cyano-2-[3-ethyl-4-oxo-5-[1-[3- (2-pyrrolidin-1-yl-acetylamino)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.24 (t, 3H); 1.76 (m, 4H); 2.60 (m, 4H); 3.05 (m, 1H); 3.25 (s, 2H); 3.91 (m, 2H); 4.23 (q, 2H); 6.88 (s, 1H); 7.20 (t, 1H); 7.29 (d, 1H); 7.61 (s, 1H); 7.73-8.01 (b, 1H); 8.12 (s, 1H); 9.70 (s, 1H); 10.45 (s, 1H) ppm. | 478.57/ 479 | INTT9 + INT22/ 5 |
| 57 | 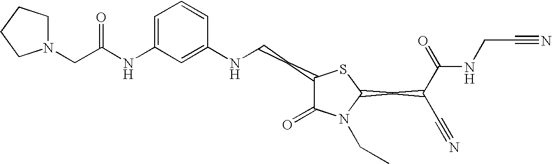 2-Cyano-N-cyanomethyl-2-[3-ethyl- 4-oxo-5-[1-[3-(2-pyrrolidin-1-yl- acetylamino)-phenylamino]-meth- (E/Z)-ylidene]-thiazolidin-(2-(E or Z))-ylidene]-acetamide | (DMSO-d6, stored via K₂CO₃, primary isomer): δ = 1.26 (t, 3H); 1.87 (m, 4H); 3.02 (m, 4H); 3.80 (s, 2H); 4.17 (d, 2H); 4.24 (q, 2H); 6.99-7.09 (m, 1H); 7.24-7.38 (m, 2H); 7.74 (s, 1H); 8.07 (d, 1H); 8.36 (t, 1H); 10.35 (s, 1H); 10.58 (d,1H) ppm. | 479.56/ 480 | INTT10 + INT22/ 5 |
| 58 | 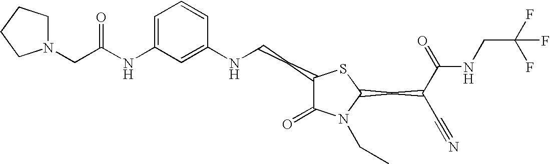 2-Cyano-2-[3-ethyl-4-oxo-5-[1-[3- (2-pyrrolidin-1-yl-acetylamino)- phenylamino]-meth-(E/Z)-ylidene]- thiazolidin-(2-(E or Z))-ylidene]-N- (2,2,2-trifluoro-ethyl)-acetamide | (DMSO-d6, stored via K₂CO₃, primary isomer): δ = 1.26 (t, 3H); 1.75 (m, 4H); 2.60 (m, 4H); 3.25 (s, 2H); 3.96 (m, 2H); 4.24 (q, 2H); 6.98 (d, 1H); 7.21-7.38 (m, 2H); 7.75 (s, 1H); 8.08 (s, 1H); 8.21 (t, 1H); 9.78 (s, 1H); 10.50 (s, 1H) ppm. | 522.55/ 523 | INTT8 + INT22/ 5 |
| 59 | 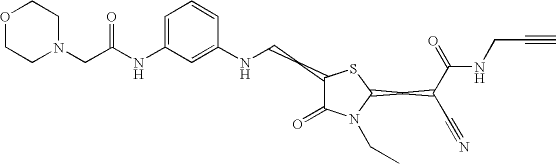 2-Cyano-2-[3-ethyl-5-[1-[3-(2- morpholin-4-yl-acetylamino)- phenylamino]-meth-(E/Z)-ylidene]- 4-oxo-thiazolidin-(2-(E or Z))- ylidene]-N-prop-2-ynyl-acetamide | (DMSO-d6, stored via K₂CO₃₃, primary isomer): δ = 1.25 (t, 3H); 2.51 (m, 4H); 3.06 (m, 1H); 3.13 (s, 2H); 3.65 (m, 4H); 3.92 (m, 2H); 4.24 (q, 2H); 6.95 (s, 1H); 7.20-7.33 (m, 2H); 7.67 (s, 1H); 7.92-8.15 (m, 2H); 9.78 (s, 1H); 10.45 (s,1H) ppm. | 494.57/ 495 | INTT9 + INT24/ 5 |
| 60 | 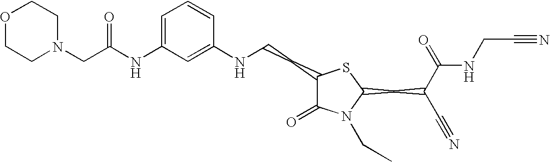 2-Cyano-N-cyanomethyl-2-[3-ethyl- 5-[1-[3-(2-morpholin-4-yl- acetylamino)-phenylamino]-meth- (E/Z)-ylidene]-4-oxo-thiazolidin-(2- (E or Z))-ylidene]-acetamide | (DMSO-d6, stored via K₂CO₃, primary isomer): δ = 1.27 (t, 3H); 2.51 (m, 4H); 3.15 (s, 2H); 3.64 (m, 4H); 4.15 (d, 2H); 4.24 (q, 2H); 6.96-7.06 (m, 1H); 7.23-7.36 (m, 2H); 7.74 (s, 1H); 8.08 (d, 1H); 8.35 (t, 1H); 9.81 (s, 1H); 10.53 (d,1H) ppm. | 495.56/ 496 | INTT10 + INT24/ 5 |
| 61 | 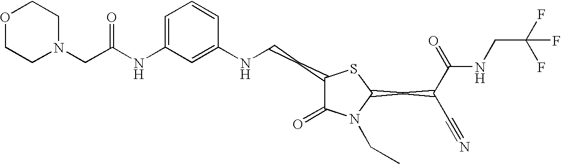 2-Cyano-2-[3-ethyl-5-[1-[3-(2- morpholin-4-yl-acetylamino)- phenylamino]-meth-(E/Z)-ylidene]- 4-oxo-thiazolidin-(2-(E or Z))- ylidene]-N-(2,2,2-trifluoro-ethyl)- acetamide | (DMSO-d6, stored via K₂CO₃, primary isomer): δ = 1.27 (t, 3H); 2.51 (m, 4H); 3.14 (s, 2H); 3.64 (m, 4H); 3.97 (m, 2H); 4.25 (q, 2H); 6.95-7.04 (m, 1H); 7.22-7.33 (m, 2H); 7.73 (s, 1H); 8.07 (d, 1H); 8.21 (t, 1H); 9.80 (s,1H); 10.50 (d, 1H) ppm. | 538.55/ 539 | INTT8 + INT24/ 5 |
| 62 | 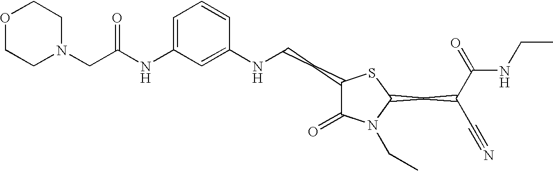 2-Cyano-N-ethyl-2-[3-ethyl-5-[1-[3- (2-morpholin-4-yl-acetylamino)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.08 (t, 3H); 1.25 (t, 3H); 2.50 (m, 4H); 3.13 (s, 2H); 3.20 (m, 2H); 3.65 (m, 4H); 4.23 (q, 2H); 6.91-7.02 (m, 1H); 7.20-7.33 (m, 2H); 7.63-7.75 (m, 2H); 8.01 (s, 1H); 9.79 (s, 1H); 10.39 (s,1H) ppm. | 484.58/ 485 | INTT7 + INT24/ 5 |
| 63 | 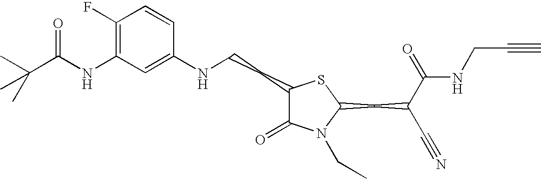 2-Cyano-2-[5-[1-[3-(2,2-dimethyl- propionylamino)-4-fluoro- phenylamino]-meth-(E/Z)-ylidene]- 3-ethyl-4-oxo-thiazolidin-(2-(E or Z))-ylidene]-N-prop-2-ynyl- acetamide | (DMSO-d6, stored via K₂CO₃, primary isomer): δ = 1.19-1.30 (m, 12H); 3.07 (m, 1H); 3.92 (m, 2H); 4.23 (q, 2H); 7.09-7.18 (m, 1H); 7.22 (t, 1H); 7.51 (m, 1H); 8.02 (d, 1H); 8.10 (t, 1H); 9.08 (s, 1H); 10.39 (d, 1H) ppm. | 469.54/ 470 | INTT9 + INT19/ 5 |
| 64 | 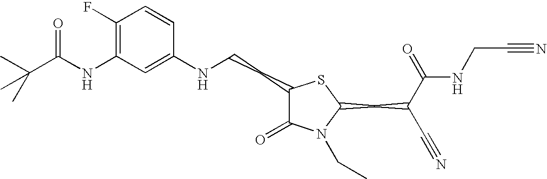 2-Cyano-N-cyanomethyl-2-[5-[1-[3- (2,2-dimethyl-propionylamino)-4- fluoro-phenylamino]-meth-(E/Z)- ylidene]-3-ethyl-4-oxo-thiazolidin- (2-(E or Z))-ylidene]-acetamide | (DMSO-d6, stored via K₂CO₃, primary isomer): δ = 1.16-1.31 (m, 12H); 4.17 (d, 2H); 4.23 (q, 2H); 7.11-7.19 (m, 1H); 7.24 (t, 1H); 7.03 (m, 1H); 8.07 (d, 1H); 8.36 (t, 1H); 9.09 (s, 1H); 10.45 (d, 1H) ppm. | 470.53/ 471 | INTT10 + INT19/ 5 |
| 65 | 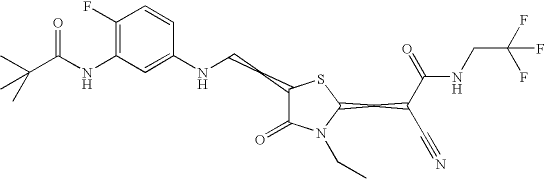 2-Cyano-2-[5-[1-[3-(2,2-dimethyl- propionylamino)-4-fluoro- phenylamino]-meth-(E/Z)-ylidene]- 3-ethyl-4-oxo-thiazolidin-(2-(E or Z))-ylidene]-N-(2,2,2-trifluoro- ethyl)-acetamide | (DMSO-d6, stored via K₂CO₃, primary isomer): δ = 1.17-1.33 (m, 12H); 3.97 (m, 2H); 4.23 (q, 2H); 7.11 (s, 1H); 7.21 (t, 1H); 7.49 (s, 1H); 8.08 (s, 1H); 8.13 (s, 1H); 9.06 (s, 1H); 10.44 (s, 1H) ppm. | 513.51/ 514 | INTT8 + INT19/ 5 |
| 66 | 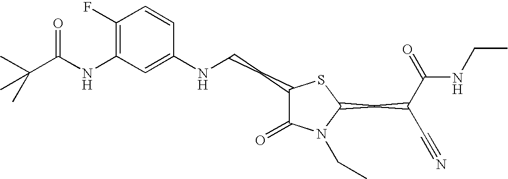 2-Cyano-2-[5-[1-[3-(2,2-dimthyl- propionylamino)-4-fluoro- phenylamino]-meth-(E/Z()-ylidene]- 3-ethyl-4-oxo-thiazolidin-(2-(E or Z))-ylidene-N-ethyl-acetamide | (DMSO-d6, stored via K₂CO₃, primary isomer): δ = 1.08 (t, 3H); 1.15-1.30 (m, 12H); 3.20 (pentuplet, 2H); 4.21 (q, 2H); 7.08-7.16 (m, 1H); 7.21 (t, 1H); 7.51 (m, 1H); 7.70 (t, 1H); 8.00 (s, 1H); 9.08 (s, 1H); 10.31 (s, 1H) ppm. | 459.54/ 460 | INTT7 + INT19/ 5 |
| 67 | 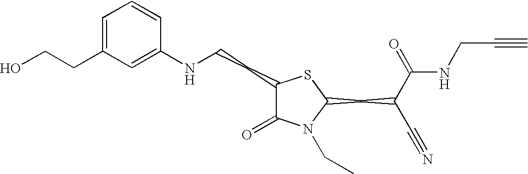 2-Cyano-2-[3-ethyl-5-[1-[3-(2- hydroxy-ethyl)-phenylamino]-meth- (E/Z)-ylidene]-4-oxo-thiazolidin-(2- (E or Z))-ylidene]-N-prop-2-ynyl- acetamide | (DMSO-d6, stored via K₂CO₃, primary isomer): δ = 1.25 (t, 3H); 2.72 (t, 2H); 3.07 (m, 1H); 3.62 (q, 2H); 3.92 (m, 2H); 4.23 (q, 2H); 4.65 (t, 1H); 6.92 (d, 1H); 7.11 (d, 1H); 7.17 (s, 1H); 7.23 (t, 1H); 8.06 (s, 1H); 8.12 (s, 1H); 10.33 (s, 1H)ppm. | 396.47/ 2397 | INTA8/ 1 |
| 68 | 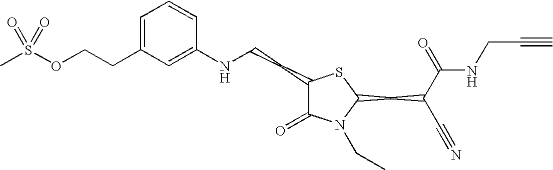 Methanesulfonic acid 2-[(3-{[2-[1- cyano-1-prop-2-ynylcarbamoyl- meth-(E or Z)-ylidene]-3-ethyl-4- oxo-thiazolidin-(5-(E/Z))- ylidenemethyl]-amino}-phenyl)- ethyl ester | (DMSO-d6, stored via K₂CO₃, primary isomer): δ = 1.26 (t, 3H); 3.01 (t, 2H); 3.07 (m, 1H); 3.12 (s, 3H); 3.93 (m, 2H); 4.24 (q, 2H); 4.43 (t, 2H); 7.00 (d, 1H); 7.18 (d, 1H); 7.23-7.34 (m, 2H); 8.09 (t, 1H); 8.16 (s, 1H); 10.32 (s, 1H) ppm. | 474.56/ 475 | 67/ 12 |
| 69 | 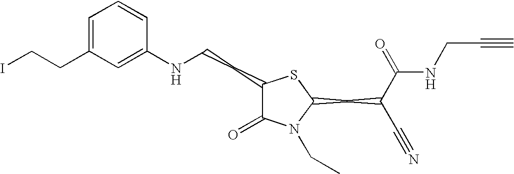 2-Cyano-2-[3-ethyl-5-[1-[3-(2-iodo- ethyl)-phenylamino]-meth-(E/Z)- ylidene]-4-oxo-thiazolidin-(2-(E or Z))-ylidene]-N-prop-2-ynyl- acetamide | (DMSO-d6, stored via K₂CO₃, primary isomer): δ = 1.25 (t, 3H); 3.06 (m, 1H); 3.13 (t, 2H); 3.50 (t, 2H); 3.92 (m, 2H); 4.21 (q, 2H); 6.96 (d, 1H); 7.16 (d, 1H); 7.20-7.32 (m, 2H); 8.08 (s, b, 1H); 8.15 (s, 1H); 10.31 (s, 1H) ppm. | 506.37/ 507 | 68/ 13 |
| 70 | 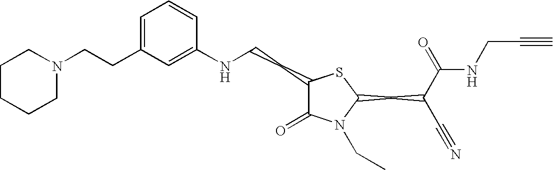 2-Cyano-2-[3-ethyl-4-oxo-5-[1-[3- (2-piperidin-1-yl-ethyl)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.25 (t, 3H); 1.39 (m, 1H); 1.50 (m, 4H); 2.30-2.52 (m, 6H); 2.72 (m, 2H); 3.07 (m, 1H); 3.92 (m, 2H); 4.24 (q, 2H); 6.93 (d, 1H); 7.11 (d, 1H); 7.17-7.29 (m, 2H); 8.04-8.18 (m, 2H); 10.30 (s, b,1H) ppm. | 463.60/ 464 | 69/ 14 |
| 71 | 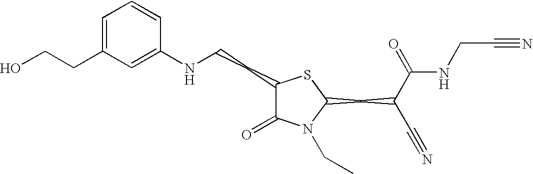 2-Cyano-N-cyanomethyl--2-[3-ethyl- 5-[1-[3-(2-hydroxy-ethyl)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.26 (t, 3H); 2.72 (t, 2H); 3.61 (q, 2H); 4.17 (d, 2H); 4.23 (q, 2H); 4.65 (t, 1H); 6.93 (d, 1H); 7.13 (d, 1H); 7.19 (s, 1H); 7.24 (t, 1H); 8.15 (s, 1H); 8.32 (t, 1H); 10.41 (s, 1H) ppm. | 397.46/ 398 | INTA8/ 1 |
| 72 | 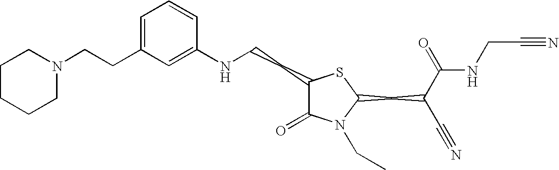 2-Cyano-N-cyanomethyl-2-[3-ethyl- 4-oxo-5-[1-[3-(2-piperidin-1-yl- ethyl)-phenylamino]-meth-(E/Z)- ylidene]-thiazolidin-(2-(E or Z))- ylidene]-acetamide | (DMSO-d6, stored via K₂CO₃, primary isomer): δ = 1.25 (t, 3H); 1.39 (m, 2H); 1.50 (m, 4H); 2.40 (m, 4H); 2.49 (t, 2H); 2.53 (t, 2H); 4.16 (d, 2H); 4.25 (q, 2H); 6.94 (d, 1H); 7.11 (d, 1H); 7.16-7.30 (m, 2H); 8.16 (s, 1H); 8.32 (s, 1H); 10.48(s, b, 1H) ppm. | 464.59/ 465 | 13/ 14 |
| 73 | 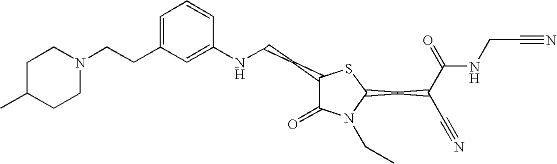 2-Cyano-N-cyanomethyl-2-]3-ethyl- 5[1-{3-[2-(4-methyl-piperidin-1-yl)- ethyl]-4-oxo-thiazolidin-(2-(E or Z))-ylidene]-acetamide | (DMSO-d6, stored via K₂CO₃, primary isomer): δ = 0.88 (d, 3H); 1.02-1.20 (m, 2H); 1.20-1.39 (m, 4H); 1.57 (d, 2H); 1.91 (t, 2H); 2.40-2.55 (m, 2H); 2.71 (t, 2H); 2.88 (d, 2H); 4.17 (m, 2H); 4.23 (q, 2H); 6.93 (d, 1H); 7.10 (d, 1H); 7.26-7.30 (m, 2H); 8.18 (s, 1H); 8.31 (s, 1H); 10.39 (s, 1H) ppm. | 478.62/ 479 | 13/ 14 |
| 74 | 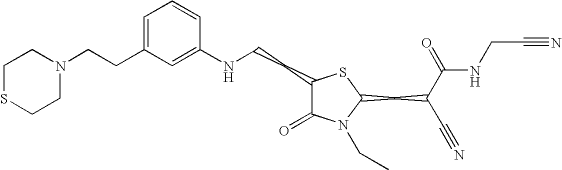 2-Cyano-N-cyanomethyl-2-[3-ethyl- 4-oxo-5-[1-[3-(2-thiomorpholin-4-yl- ethyl)-phenylamino]-meth-(E/Z)- ylidene]-thiazolidin-(2-(E or Z))- ylidene]-acetamide | (DMSO-d6, stored via K₂CO₃, primary isomer): δ = 1.25 (t, 3H); 2.51-2.67 (m, 6H); 2.67-2.81 (m, 6H); 4.17 (m, 2H); 4.24 (q, 2H); 6.93 (d, 1H); 7.11 (d, 1H); 7.20 (s, 1H); 7.24 (t, 1H); 8.17 (s, 1H); 8.32 (s, 1H); 10.39 (s, 1H) ppm. | 482.63/ 483 | 13/ 14 |
| 75 | 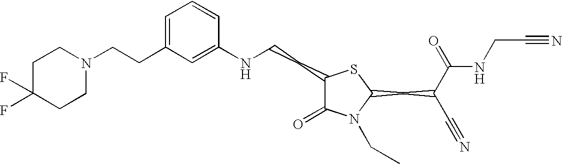 2-Cyano-N-cyanomethyl-2-[5-[1-{3- [2-(4,4-difluoro-piperidin-1-yl)- ethyl]-phenylamino}-meth-(E/Z)- ylidene]-3-ethyl-4-oxo-thiazolidin- (2-(E or Z))-ylidene]-acetamide | (DMSO-d6, stored via K₂CO₃, primary isomer): δ = 1.26 (t, 3H); 1.85-2.07 (m, 4H); 2.50-2.67 (m, 6H); 2.75 (m, 2H); 4.17 (d, 2H); 4.26 (q, 2H); 6.93 (d, 1H); 7.10 (m, 1H); 7.15-7.31 (m, 2H); 8.18 (s, 1H); 8.28 (s, b, 1H); 10.39(s, 1H) ppm. | 500.57/ 501 | 13/ 14 |
| 76 | 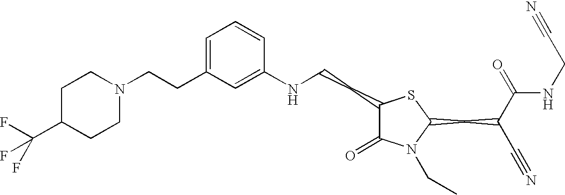 2-Cyano-N-cyanomethyl-2-[3-ethyl- 4-oxo-5-[1-{3-[2-(4-trifluoromethyl- piperidin-1-yl)-ethyl]-phenylamino}- meth-(E/Z)-ylidene]-thiazolidin-(2- (E or Z))-ylidene]-acetamide | (DMSO-d6, stored via K₂CO₃, primary isomer): δ = 1.24 (t, 3H); 1.36-1.52 (m, 2H); 1.78 (d, 2H); 1.99 (t, 2H); 2.13-2.36 (m, 1H); 2.54 (m, 2H); 2.73 (m, 2H); 3.01 (d, 2H); 4.16 (m, 2H); 4.23 (q, 2H); 6.92 (d, 1H); 7.01-7.30 (m, 3H); 8.19 (s, 1H); 8.27 (s, 1H); 10.40 (s, 1H) ppm. | 532.59/ 533 | 13/ 14 |
| 77 | 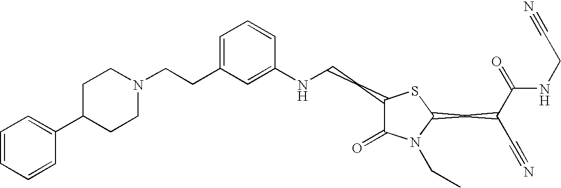 2-Cyano-N-cyanomethyl-2-[3-ethyl- 4-oxo-5-[1-{3-[2-(4-phenyl- piperidin-1-yl)-ethyl]-phenylamino}- meth-(E/Z)-ylidene]-thiazolidin-(2- (E or Z))-ylidene]-acetamide | (DMSO-d6, stored via K₂CO₃ primary isomer selected signals): δ = 1.24 (t, 3H); 1.56-1.99 (m, 4H); 2.09 (t, 2H); 2.56 (m, 2H); 2.76 (m, 2H); 3.04 (d, 2H); 4.13 (d, 2H); 4.24 (q, 2H); 6.94 (d, 1H); 7.01-7.40 (m, 8H); 8.10-8.35 (m,2H); 10.40 (s, 1H) ppm. | 540.69/ 541 | 13/ 14 |
| 78 | 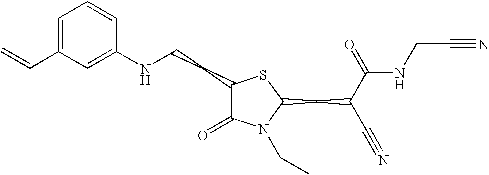 2-Cyano-N-cyanomethyl-2-[3-ethyl- 4-oxo-5-[1-(3-vinyl-phenylamino)- meth-(E/Z)-ylidene]-thiazolidin-(2- (E or Z))-ylidene]-acetamide | (DMSO-d6, store via K₂CO₃, primary isomer): δ = 1.25 (t, 3H); 4.17 (d, 2H); 4.23 (q, 2H); 5.31 (d, 1H); 5.90 (d, 1H); 6.74 (dd, 1H); 7.13-7.26 (m, 2H); 7.32 (t, 1H); 7.44 (s, 1H); 8.20 (s, 1H); 8.34 (t, 1H); 10.41 (s, 1H) ppm. | 379.44/ 380 | 13/ 14 |
| 79 | 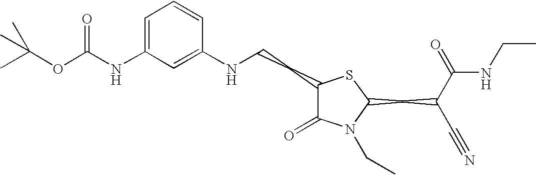 (3-{[2-[1-Cyano-1-ethylcarbamoyl- meth-(E/Z)-ylidene]-3-ethyl-4- oxo-thiazolidin-(5-(E/Z))- ylidenemethyl]-amino}-phenyl)- carbamic acid tert-butyl ester | (DMSO-d6, stored via K₂CO₃, primary isomer): δ = 1.06 (t, 3H); 1.23 (t, 3H); 1.49 (s, 9H); 3.20 (m, 2H); 4.22 (q, 2H); 6.88 (d, 1H); 7.14 (d, 1H); 7.20 (t, 1H); 7.55 (s, 1H); 7.70 (t, 1H); 7.99 (d, 1H); 9.43 (s, 1H); 10.39 (d, 1H) ppm. | 457.56/ 458 | INTA9/ 1 |
| 80 | 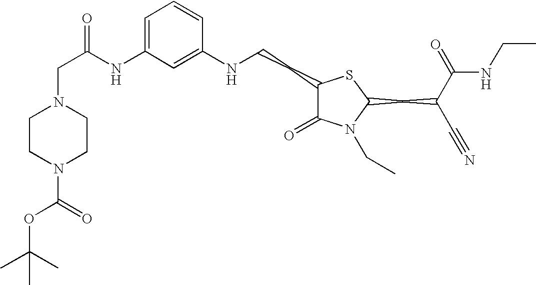 4-[(3-{[2-[1-Cyano-1- ethylcarbamoyl-meth-(E or Z)- ylidene]-3-ethyl-4-oxo-thiazolidin- (5-(E/Z))-ylidenemethyl]-amino}- phenylcarbamoyl)-methyl]- piperiazine-1-carboxylic acid tert- butyl ester | (DMSO-d6, stored via K₂CO₃, primary isomer): δ = 1.08 (t, 3H); 1.25 (t, 3H); 1.40 (s,9H); 2.48 (m, 4H); 3.17 (s, 3H); 3.21 (m, 2H); 3.40 (m, 4H); 4.23 (q, 2H); 6.98 (m, 1H); 7.21-7.34 (m, 2H); 7.64-7.77 (m, 2H); 8.02 (s, 1H); 9.80 (s, 1H); 10.39 (s, 1H) ppm. | 583.71/ 584 | 7/8 |
| 81 | 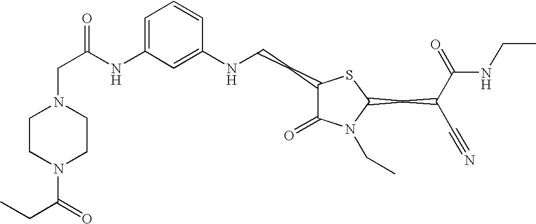 2-Cyano-N-ethyl-2-[3-ethyl-4-oxo- 5-[1-{3-[2-(4-propionyl-piperazin-1- yl)-acetylamino]-phenylamino}- meth-(E/Z)-ylidene]-thiazolidin-(2- (E or Z))-ylidene]-acetamide | (DMSO-d6, stored via K₂CO₃, primary isomer): δ = 1.00 (t, 3H); 1.08 (t, 3H); 1.24 (t, 3H); 2.31 (q, 2H); 2.40-2.60 (m, 4H); 3.12-3.28 (m, 4H); 3.50 (m, 4H); 4.22 (q, 2H); 6.98 (m, 1H); 7.21-7.33 (m, 2H); 7.61-7.76 (m, 2H); 8.01(s, 1H); 9.80 (s, 1H); 10.39 (s, 1H) ppm. | 539.66/ 540 | 80/9 |
| 82 | 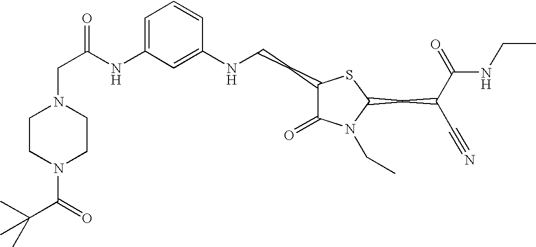 2-Cyano-2-[5-[1-(3-{2-[4-(2,2- dimethyl-propionyl)-piperazin-1-yl]- acetylamino}-phenylamino)-meth- (E/Z)-ylidene]-3-ethyl-4-oxo- thiazolidin-(2-(E or Z))-ylidene]-N- ethyl-acetamide | (DMSO-d6, stored via K₂CO₃, primary isomer): δ = 1.08 (t, 3H); 1.20 (s, 9H); 1.26 (t, 3H); 2.42-2.58 (m, 4H); 3.17 (s, 2H); 3.21 (m, 2H); 3.61 (m, 4H); 4.22 (q, 2H); 6.99 (d, 1H); 7.21-7.32 (m, 2H); 7.62-7.76 (m,2H); 8.01 (s, 1H); 9.80 (s, 1H); 10.40 (s, 1H) ppm. | 567.72/ 568 | 80/ 10 |
| 83 | 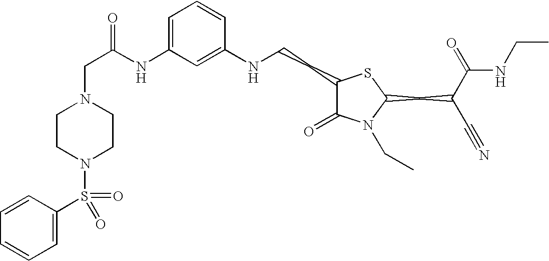 2-[5-[1-{3-[2-(4-Benzenesulfonyl- piperazin-1-yl)-acetylamino]- phenylamino}-meth-(E/Z)-ylidene]- 3-ethyl-4-oxo-thiazolidin-(2-(E or Z))-ylidene]-2-Cyano-N-ethyl- acetamide | (DMSO-d6, stored via K₂CO₃, primary isomer): δ = 1.08 (t, 3H); 1.25 (t, 3H); 2.60 (m, 4H); 2.99 (m, 4H); 3.15 (s, 2H); 3.20 (m, 2H); 4.22 (q, 2H); 6.92 (s, 1H); 7.16-7.28 (m, 2H); 7.52-7.82 (m, 7H); 8.00 (s, 1H); 9.67(s, 1H); 10.36 (s, 1H) ppm. | 623.76/ 624 | 80/ 10 |
| 84 | 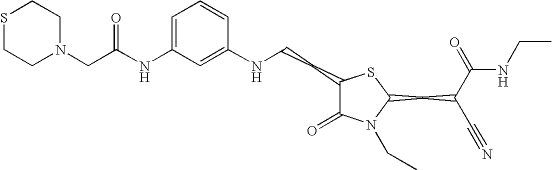 2-Cyano-N-ethyl-2-[3-ethyl-4-oxo- 5-[1-[3-(2-thiomorpholin-4-yl- acetylamino)-phenylamino]-meth- (E/Z)-ylidene]-thiazolidin-(2-(E or Z))-ylidene]-acetamide | (DMSO-d6, stored via K₂CO₃,primary isomer): δ = 1.07 (t, 3H); 1.24 (t, 3H); 2.70 (m, 4H); 2.79 (m, 4H); 3.17 (s, 2H); 3.20 (m, 2H); 4.22 (q, 2H); 6.97 (d, 1H); 7.20-7.34 (m, 2H); 7.55-7.77 (m, 2H); 8.05 (s, 1H); 9.71 (s, 1H); 10.39 (s, 1H)ppm. | 500.65/ 501 | 7/8 |
| 85 | 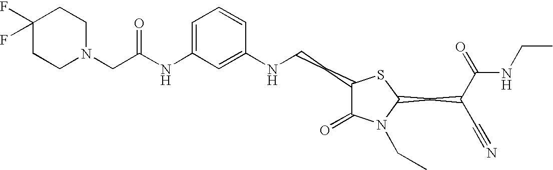 2-Cyano-2-[5-[1-{3-[2-(4,4-difluoro- piperidin-1-yl)-acetylamino]- phenylamino}-meth-(E/Z)-ylidene]- 3-ethyl-4-oxo-thiazolidin-(2-(E or Z))-ylidene]-N-ethyl-acetamide | (DMSO-d6, stored via K₂CO₃, primary isomer): δ = 1.08 (t, 3H); 1.25 (t, 3H); 1.94-2.14 (m, 4H); 2.67 (m, 4H); 3.12-3.28 (m, 4H); 4.22 (q, 2H); 6.98 (d, 1H); 7.21-7.35 (m, 2H); 7.60-7.77 (m, 2H); 8.01 (s, 1H); 9.79 (s, 1H); 10.39(s, 1H) ppm. | 518.59/ 519 | 7/8 |
| 86 | 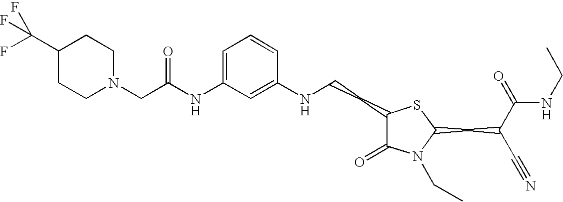 2-Cyano-N-ethyl-2-[3-ethyl-4-oxo- 5-[1-{3-[2-(4-trifluoromethyl- piperidin-1-yl)-acetylamino]- phenylamino}-meth-(E/Z)-ylidene]- thiazolidin-(2-(E or Z))-ylidene]- acetamide | (DMSO-d6, stored via K₂CO₃, primary isomer): δ = 1.08 (t, 3H); 1.25 (t, 3H); 1.50-1.70 (m, 2H); 1.80 (d, 2H); 2.12-2.37 (m, 3H); 2.95 (d, 2H); 3.15 (s, 2H); 3.21 (m, 2H); 4.22 (q, 2H); 6.99 (d, 1H); 7.20-7.35 (m, 2H); 7.64-7.76 (m, 2H); 8.01 (d, 1H); 9.73 (s, 1H); 10.39 (d, 1H) ppm. | 550.61/ 551 | 7/8 |
| 87 | 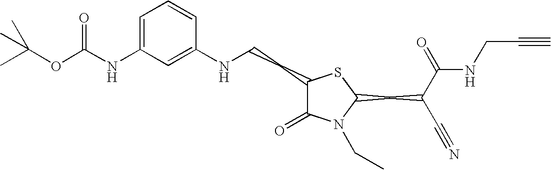 (3-{[2-[1-Cyano-1-prop-2- ynylcarbamoyl-meth-(E or Z)- ylidene]-3-ethyl-4-oxo-thiazolidin- (5-(E/Z))-ylidenemethyl]-amino}- phenyl)-carbamic acid tert-butyl ester | (DMSO-d6, stored via K₂CO₃, primary isomer): δ = 1.25 (t, 3H); 1.49 (s, 9H); 3.06 (m, 1H); 3.92 (m, 2H); 4.22 (q, 2H); 6.89 (d, 1H); 7.06 (d, 1H); 7.20 (t, 1H); 7.55 (s, 1H); 7.94-8.13 (m, 2H); 9.43 (s, 1H); 10.44 (s, 1H) ppm. | 367.43/ 368 | 87/6 |
| 88 | 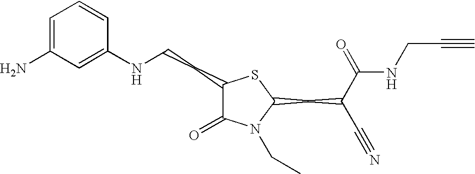 2-[5-[1-(3-Amino-phenylamino)- meth-(E/Z)-ylidene]-3-ethyl-4-oxo- thiazolidin-(2-(E or Z))-ylidene]-2- cyano-N-prop-2-ynyl-acetamide | (DMSO-d6, stored via K₂CO₃, primary isomer: δ = 1.25 (t, 3H); 3.06 (m, 1H); 3.92 (m, 2H); 4.23 (q, 2H); 6.27 (d, 1H); 6.99-7.09 (m, 2H); 7.29 (t, 1H); 8.04 (d, 1H); 8.13 (t, 1H); 8.65 (b, 3H); 10.40 (d, 1H) ppm. | 367.43/ 368 | 87/6 |
| 89 | 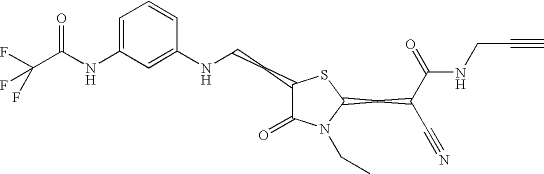 2-Cyano-2-[3-ethyl-4-oxo-5-[1-[3- (2,2,2-trifluoro-acetylamino)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.25 (t, 3H); 3.08 (m, 1H); 3.93 (m, 2H); 4.24 (q, 2H); 7.17 (m, 1H); 7.32-7.40 (m, 2H); 7.70 (s, 1H); 8.05 (s, 1H); 8.11 (t, 1H); 10.50 (s, 1H); 11.29 (s, 1H) ppm. | 463.44/ 464 | 87/9 |
| 90 | 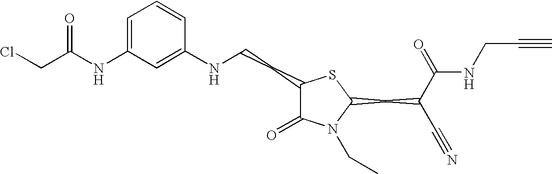 2-[5-[1-[3-(2-Chloro-acetylamino)- phenylamino]-meth-(E/Z)-ylidene]- 3-ethyl-4-oxo-thiazolidin-(2-(E or Z))-ylidene]-2-cyano-N-prop-2-ynyl- acetamide | (DMSO-d6, stored via K₂CO₃, primary isomer): δ = 1.26 (t, 3H); 3.06 (m, 1H); 3.92 (m, 2H); 4.15-4.30 (m, 4H); 7.02 (d, 1H); 7.20 (d, 1H); 7.30 (t, 1H); 7.68 (s, 1H); 7.99-8.15 (m, 2H); 10.36 (s, 1H), 10.48 (s, 1H) ppm. | 443.92/ 444 | 87/9 |
| 91 | 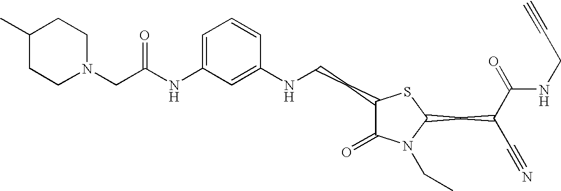 2-Cyano-2-[3-ethyl-5-[1-{3-[2-(4- methyl-piperidin-1-yl)-acetylamino]- phenylamino}-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 0.91 (d, 3H); 1.20-1.37 (m, 6H); 1.60 (d, 2H); 2.13 (t, 2H); 2.83 (d, 2H); 3.06 (m, 1H); 3.09 (s, 2H); 3.92 (m, 2H); 4.24 (q, 2H); 6.99 (d, 1H); 7.21-7.35 (m, 2H); 7.71 (s,1H); 7.98-8.15 (m, 2H); 9.70 (s, 1H); 10.45 (s, 1H) ppm. | 506.63/ 507 | 90/8 |
| 92 | 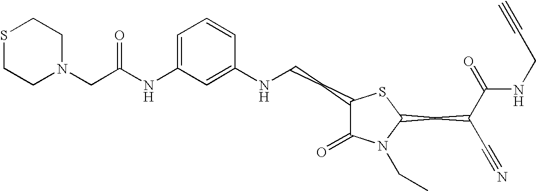 2-Cyano-2-[3-ethyl-4-oxo-5-[1-[3- (2-thiomorpholin-4-yl-acetylamino)- phenylamino]-meth-(E/Z)-ylidene]- thiazolidin-(2-(E or Z))-ylidene]-N- prop-2-ynyl-acetamide | (DMSO-d6, stored via K₂CO₃, primary isomer): δ = 1.25 (t, 3H); 2.70 (m, 4H); 2.79 (m, 4H); 3.08 (m, 1H); 3.18 (s, 2H); 3.93 (m, 2H); 4.24 (q, 2H); 6.99 (d, 1H); 7.21-7.36 (m, 2H); 7.73 (s, 1H); 8.00-8.15 (m, 2H); 9.74 (s, 1H); 10.45(s, 1H) ppm. | 510.64/ 511 | 90/8 |
| 93 | 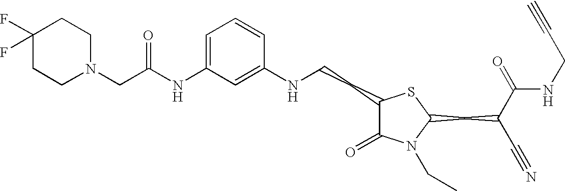 2-Cyan-2-[5-[1-{3-[2-(4,4-difluoro- piperidin-1-yl)-acetylamino]- phenylamino}-meth-(E/Z)-ylidene]- 2-ethyl-4-oxo-thiazolidin-(2-(E or Z))-ylidene]-N-prop-2-ynyl- acetamide | (DMSO-d6, stored via K₂CO₃, primary isomer): δ = 1.26 (t, 3H); 1.97-2.13 (m, 4H); 2.68 (m, 4H); 3.07 (m, 1H); 3.24 (s, 2H); 3.92 (m, 2H); 4.22 (q, 2H); 6.99 (d, 1H); 7.22-7.36 (m, 2H); 7.73 (s, 1H); 8.00-8.15 (m, 2H); 9.80 (s, 1H); 10.47 (s, 1H) ppm. | 528.59/ 529 | 90/8 |
| 94 | 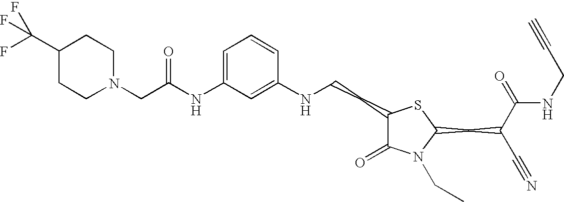 2-Cyano-2-[3-ethyl-4-oxo-5-[1-{3- [2-(4-trifluoromethyl-piperidin-1-yl)- acetylamino]-phenylamino}-meth- (E/Z)-ylidene]-thiazolidin-(2-(E or Z))-ylidene]-N-prop-2-ynyl- acetamide | (DMSO-d6, stored via K₂CO₃, primary isomer): δ = 1.24 (t, 3H); 1.50-1.70 (m,2 H); 1.79 (d, 2H); 2.13-2.36 (m, 3H); 2.95 (d, 2H); 3.08 (m, 1H); 3.15 (s, 2H); 3.92 (m, 2H); 4.23 (q, 2H); 6.99 (d, 1H); 7.20-7.37 (m,2H); 7.71 (s, 2H); 7.97-8.19 (m, 2H); 9.75 (s, 1H); 10.46 (x, 1H) ppm. | 560.60/ 561 | 90/8 |
| 95 | 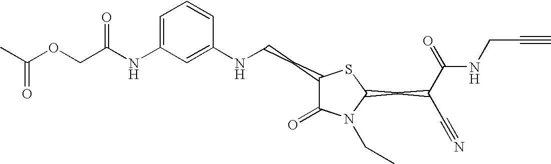 Acetic acid (3-{[2-[1-cyano-1- (cyanomethyl-carbamoyl)-meth-(E or Z)-ylidene]-3-ethyl-4-oxo- thiazolidin-(5-(E/Z))-ylidenemethyl]- amino}-phenylcarbamoyl)-methyl ester | (DMSO-d6, stored via K₂CO₃, primary isomer): δ = 1.26 (t, 3H); 2.13 (s, 3H); 4.17 (d, 2H); 4.24 (q, 2H); 4.66 (s, 2H); 7.02 (d, 1H); 7.20 (d, 1H); 7.30 (t, 1H); 7.69 (s, 1H); 8.06 (d, 1H); 8.35 (t, 1H); 10.17 (s, 1H); 10.54(d, 1H) ppm. | 468.49/ 469 | INTT10 + INT27/ 5 |
| 96 | 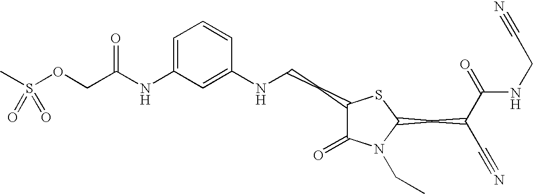 Methanesulfonic acid (3-{[2-[1- cyano-1-(cyanomethyl-carbamotl)- meth-(E or Z)-yliene]-3-ethyl-4- oxo-thiazolidin-(5-(E/Z))- ylidenemethyl]-amino}- phenylcarbamoyl)-methyl ester | (DMSO-d6, stored via K₂CO₃, primary isomer): δ = 1.24 (t, 3H); 3.31 (s, 3H); 4.17 (d, 2H); 4.25 (q, 2H); 4.88 (s, 2H); 7.07 (d, 1H); 7.24 (d, 1H); 7.31 (t, 1H); 7.70 (s, 1H); 8.07 (s, b, 1H); 8.37 (s, 1H); 10.26 (s,1H); 10.57 (s, 1H) ppm. | 504.55/ 505 | 11/ 12 |
| 97 | 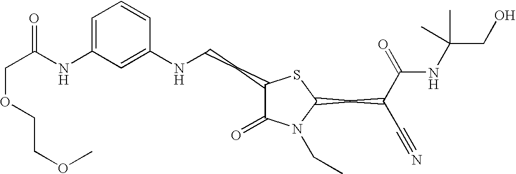 2-Cyano-2-[3-ethyl-5-[1-{3-[2-(2- methoxy-ethoxy)-acetylamino]- phenylamino}-meth-(E/Z)-ylidene]- 4-oxo-thiazolidin-(2-(E or Z))- ylidene]-N-(2-hydroxy-1,1-dimthyl- ethyl)-acetamide | (DMSO-d6, stored via K₂CO₃, primary isomer): δ = 1.24 (t, 3H); 1.30 (s, 6H); 3.31 (s, 3H); 3.38 (d, 2H); 3.55 (m, 2H); 3.69 (m, 2H); 4.09 (s, 2H); 4.21 (q, 2H); 5.20 (t, 1H); 6.70 (s, 1H); 7.01 (m, 1H); 7.23-7.32 (m, 2H); 7.74 (s, 1H); 8.02 (d, 1H); 9.70 (s, 1H); 10.40 (d, 1H) ppm. | 517.60/ 518 | INTA5/ 1 |
| 98 | 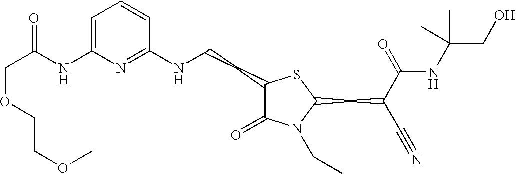 2-Cyano-2-[3-ethyl-5-[1-{6-[2-(2- methoxy-ethoxy)-acetylamino]- pyridin-2-ylamino}-meth-(E/Z)- ylidene]-4-oxo-thiazolidin-(2-(E or Z))-ylidene]-N-(2-hydroxy-1,1- dimethyl-ethyl)-acetylamide | (DMSO-d6, stored via K₂CO₃, primary isomer): δ = 1.23 (t, 3H); 1.30 (s, 6H); 3.32 (s, 3H); 3.38 (d, 2H); 3.51 (m, 2H); 3.68 (m, 2H); 4.15 (s, 2H); 4.20 (q, 2H); 5.71 (t, 1H); 6.71 (s, 1H); 6.80 (d,1H); 7.69-7.80 (m, 2H); 8.69 (s, 1H); 9.95 (s, 1H); 10.75 (s, 1H) ppm. | 518.59/ 519 | INTA6/ 1 |
| 99 | 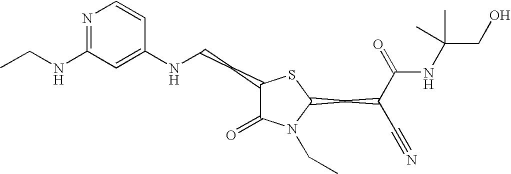 2-Cyano-2-[3-ethyl-5-[1-(2- ethylamino-pyridin-4-ylamino)- meth-(E/Z)-ylidene]-4-oxo- thiazolidin-(2-(E or Z))-ylidene]-N- (2-hydroxy-1,1-dimethyl-ethyl)- acetamide | (DMSO-d6, stored via K₂CO₃, primary isomer): δ = 1.11 (t, 3H); 1.24 (t, 3H); 1.30 (s, 6H); 3.22 (m, 2H); 3.28 (d, 2H); 4.20 (q, 2H); 5.20 (t, 1H); 6.23 (s, 1H); 6.37-6.49 (m, 2H); 6.71 (s, 1H); 7.83 (d, 1H); 8.00 (s, 1H); 10.20 (s,1H) ppm. | 430.53/ 431 | INTA2/ 1 |
| 100 | 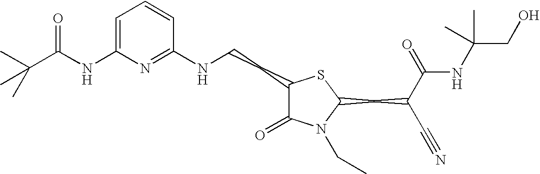 2-Cyano-2-[5-[1-[6-(2,2-dimethyl- propionylamino)-pyridin-2-ylamino]- meth-(E/Z)-ylidene]-3-ethyl-4-oxo- thiazolidin-(2-(E or Z))-ylidene]-N- (2-hydroxy-1,1-dimethyl-ethyl)- acetamide | (DMSO-d6, stored via K₂CO₃, primary isomer): δ = 1.06 (t, 3H); 1.25 (s,9H); 1.30 (s, 6H); 3.39 (d, 2H); 4.21 (q, 2H); 5.20 (t, 1H); 6.72 (s, 1H); 6.79 (dd, 1H); 7.65-7.77 (m, 2H); 8.55 (s, 1H); 9.68 (s, 1H); 10.68 (s, 1H) ppm. | 486.59/ 487 | INTA3/ 1 |
| 101 | 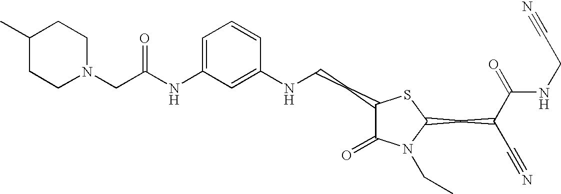 2-Cyano-N-cyanomethyl-2-[3-ethyl- 5-[1-{3-[2-(4-methyl-piperidin-1-yl)- acetylamino]-phenylamino}-meth- (E/Z)-ylidene]-4-oxo-thiazolidin(2- (E or Z))-ylidene]-acetamide | (DMSO-d6, stored via K₂CO₃, primary isomer): δ = 0.91 (d, 3H); 1.15-1.40 (m, 6H); 1.60 (d, 2H); 2.11 (t, 2H); 2.81 (d, 2H); 3.09 (s, 2H); 4.17 (d, 2H); 4.23 (q, 2H); 7.00 (d, 1H); 7.21-7.35 (m, 2H); 7.73 (s, 1H); 8.10 (s,1H); 8.34 (s, 1H); 9.71 (s, 1H); 10.51 (s, 1H) ppm. | 507.62/ 508 | 96/ 8 |
| 102 | 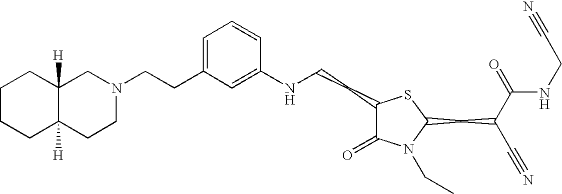 2-Cyano-N-cyanomethyl-2-[3-ethyl- 5-[1-{3-[(4a-(R or S),8a-(R or S))-2- (octahydro-isoquinolin-2-yl)-ethyl]- phenylamino}-meth-(E/Z)-ylidene]- 4-oxo-thiazolidin-(2-(E or Z))- ylidene]-acetamide | (DMSO-d6, stored via K₂CO₃, primary isomer): δ = 0.74-1.02 (m, 3H); 1.02-1.35 (m, 7H); 1.43-1.88 (m, 6H); 1.98 (m, 1H); 2.35-2.53 (m, 2H); 2.63-3.06 (m, 4H); 4.16 (d, 2H); 4.24 (q, 2H); 6.93 (d, 1H); 7.11 (d, 1H); 7.18-7.31 (m, 2H); 8.17 (s, 1H); 8.36 (t, 1H); 10.39 (s, 1H) ppm. | 518.69/ 519 | 13/ 14 |
| 103 | 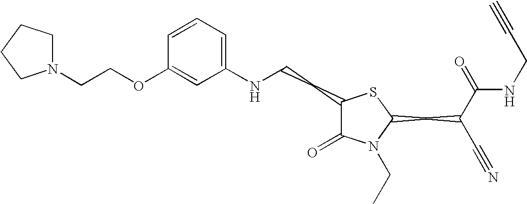 2-Cyano-2-[3-ethyl-4-oxo-5-[1-[3- (2-pyrrolidin-1-yl-ethoxy)- phenylamino]-meth-(E/Z)-ylidene]- thiazolidin-(2-(E or Z))-ylidene]-N- prop-2-ynyl-acetamide | ¹H-NMR (DMSO- d6, 300 MHz) (selected peaks) δ = 1.26 (m, 3H); 1.71 (m, 4H); 2.79 (m, 2H); 3.06 (m, 1H); 3.94 (m, 2H); 4.10 (m, 2H); 4.28 (m, 2H); 6.63 (dd, 1H); 6.89 (m, 2H); 7.22 (m, 1H); 8.13 (m, 2H); 10.28 (s, 1H). | 465.58/ 466 | INTA14/ 1 |
| 104 | 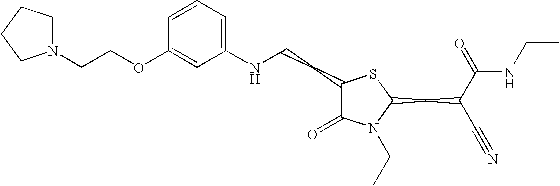 2-Cyano-N-ethyl-2-[3-ethyl-4-oxo- 5-[1-[3-(2-pyrrolidin-1-yl-ethoxy)- phenylamino]-meth-(E/Z)-ylidene]- thiazolidin-(2-(E or Z))-ylidene]- acetamide | ¹H-NMR (DMSO- d6, 300 MHz) (selected peaks) δ = 1.02 (m, 3H); 1.25 (m, 3H); 1.68 (m, 4H); 2.79 (m, 2H); 3.21 (m, 2H); 4.09 (m, 2H); 4.22 (m, 2H); 6.62 (dd, 1H); 6.88 (m, 2H); 7.22 (m, 1H); 7.70 (m, 1H); 8.10 (s, 1H); 10.18 (s, 1H). | 455.58/ 456 | INTA14/ 1 |
| 105 | 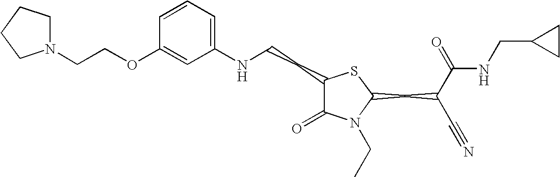 2-Cyano-N-cyclopropylmethyl-2-[3- ethyl-4-oxo-5-[1-[3-(2-pyrrolidin-1- yl-ethoxy)-phenylamino]-meth- (E/Z)-ylidene]-thiazolidin-(2-(E or Z))-ylidene]-acetamide | ¹H-NMR (DMSO- d6, 300 MHz) (selected peaks) δ = 0.22 (m, 2H); 0.40 (m, 2H); 1.02 (m, 1H); 1.26 (m, 3H); 1.69 (m, 4H); 2.79 (m, 2H); 3.03 (m, 2H); 4.09 (m, 2H); 4.22 (m, 2H); 6.62 (dd, 1H); 6.88 (m, 2H); 7.24 (m, 1H); 7.73 (m, 1H); 8.10 (s, 1H);10.19 (s, 1H). | 481.62/ 482 | INTA14/ 1 |
| 106 | 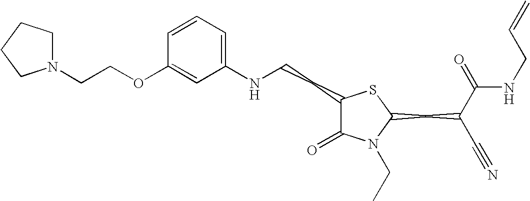 N-Allyl-2-cyano-2-[3-ethyl-4-oxo-5- [1-[3-(2-pyrrolidin-1-yl-ethoxy)- phenylamino]-meth-(E/Z)-ylidene]- thiazolidin-(2-(E or Z))-ylidene]- acetamide | ¹H-NMR (DMSO- d6, 300 MHz) (selected peaks) δ = 1.23 (m, 3H); 1.68 (m, 4H); 2.80 (m, 2H); 3.79 (m, 2H); 4.09 (m, 2H); 4.21 (m, 2H); 5.08 (dd, 1H); 5.11 (dd, 1H); 5.83 (m, 1H); 6.62 (dd, 1H); 6.88 (m, 1H); 7.22 (m, 1H); 7.98 (m, 1H); 8.12 (s, 1H); 10.20 (s, 1H). | 467.59/ 468 | INTA14/ 1 |
| 107 | 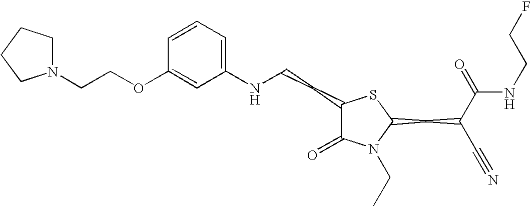 2-Cyano-2-[3-ethyl-4-oxo-5-[1-[3- (2-pyrrolidin-1-yl-ethoxy)- phenylamino]-meth-(E/Z)-ylidene]- thiazolidin-(2-(E or Z))-ylidene]-N- (2-fluoro-ethyl)-acetamide | ¹H-NMR (DMSO- d6, 300 MHz) (selected peaks) δ = 1.27 (m, 3H); 1.70 (m, 4H); 2.81 (m, 2H); 3.42 (m, 1H); 3.50 (m, 1H); 4.10 (m, 2H); 4.20 (m, 2H); 4.42 (m, 1H); 4.54 (m, 1H); 6.63 (dd, 1H); 6.88 (m, 2H); 7.21 (m, 1H); 7.80 (m, 1H); 8.12 (s, 1H);10.22 (s, 1H). | 473.57/ 474 | INTA14/ 1 |
| 108 | 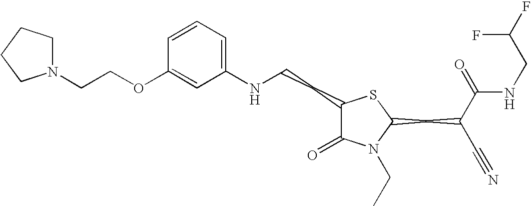 2-Cyano-N-(2,2-difluoro-ethyl)-2-[3- ethyl-4-oxo-5-[1-[3-(2-pyrrolidin-1- yl-ethoxy)-phenylamino]-meth- (e/Z)-ylidene]hiazolidin-(2-(E or Z))-ylidene]-acetamide | ¹H-NMR (DMSO- d6, 300 MHz) (selected peaks) δ = 1.27 (m, 3H); 1.69 (m, 4H); 2.79 (m, 2H); 3.58 (m, 2H); 4.10 (m, 2H); 4.25 (m, 2H); 6.09 (tt, 1H); 6.65 (dd, 1H); 6.89 (m, 2H); 7.24 (m, 1H); 7.97 (m, 1H); 8.14 (s, 1H); 10.28 (s, 1H). | 491.56/ 492 | INTA14/ 1 |
| 109 | 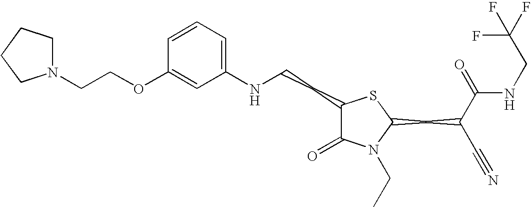 2-Cyano-2-[3-ethyl-4-oxo-5-[1-[3- (2-pyrrolidin-1-yl-ethoxy)- phenylamino]-meth-(E/Z)-ylidene]- thiazolidin-(2-(E or Z))-ylidene]-N- (2,2,2-trifluoro-ethyl)-acetamide | ¹H-NMR (DMSO-d d6, 300 MHz) (selected peaks) δ = 1.22 (m, 3H); 1.70 (m, 4H); 2.81 (m, 2H); 3.96 (m, 2H); 4.09 (m, 2H); 4.22 (m, 2H); 6.66 (dd, 1H); 6.88 (m, 2H); 7.22 (m, 1H); 8.18 (m, 2H); 10.29 (s, 1H). | 509.55/ 510 | INTA14/ 1 |
| 110 | 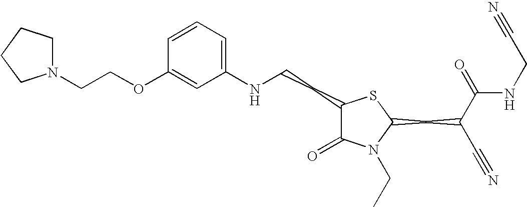 2-Cyano-N-cyanomethyl-2-[3-ethyl- 4-oxo-5-[1-[3-(2-pyrrolidin-1-yl- ethoxy)-phenylamino]-meth-(E/Z)- ylidene]-thiazolidin-(2-(E or Z))- ylidene]-acetamide | ¹H-NMR (DMSO- d6, 300 MHz) (selected peaks) δ = 1.26 (m, 3H); 1.70 (m, 4H); 2.81 (m, 2H); 4.09 (m, 2H); 4.19 (d, 2H); 4.23 (m, 2H); 6.64 (dd, 1H); 6.89 (m, 2H); 7.25 (m, 1H); 8.18 (s, 1H); 8.35 (m, 1H); 10.32 (s, 1H). | 466.56/ 467 | INTA14/ 1 |
| 111 | 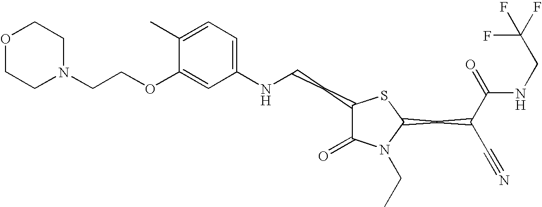 2-Cyano-2-[3-ethyl-5-[1-[4-methyl- 3-(2-morpholin-4-yl-ethoxy)- phenylamino]-meth-(E/Z)-ylidene]- 4-oxo-thiazolidin-(2-(E or Z))- ylidene]-N-(2,2,2-trifluoro-ethyl)- acetamide | ¹H-NMR (CDCl₃, 300 MHz) δ = 1.40 (m, 3H); 2.17 (s, 3H); 2.61 (m, 4H); 2.87 (m, 2H); 3.71 (m, 4H); 3.97 (m, 2H); 4.07 (m, 2H); 4.35 (m, 2H); 6.60 (m, 3H); 7.09 (d, 1H); 7.57 (m, 1H); 10.50 (d, 1H). | MW; 539.58 MS (ESI) [M+ 1]⁺: 540 | INTA11/ 1 |
| 112 | 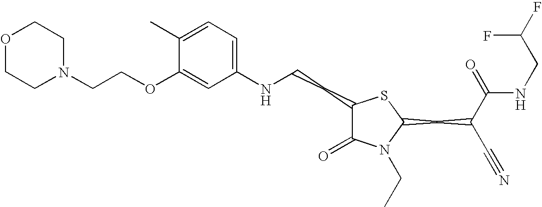 2-Cyano-N-(2,2-difluoro-ethyl)-2-[3- ethyl-5-[1-[4-(3-hydroxy-2- piperidin-2-yl-ethyl)-phenylamino]- meth-(E/Z)-ylidene]-4-oxo- thiazolidin-(2-(E or Z))-ylidene]- acetamide | ¹H-NMR (DMSO- d6,300 MHz) (selected peaks) δ = 1.22 (m, 3H); 2.10 (s, 3H); 2.72 (m, 2H); 3.58 (m, 4H); 4.11 (m, 2H); 4.21 (m, 2H); 6.05 (tt, 1H); 6.79 (dd, 1H); 6.91 (s, 1H); 7.08 (d, 1H); 7.95 (m, 1H); 8.16 (s, 1H); 10.27 (s, 1H). | MW: 521.59 MS (ESI) [M + 1]⁺: 522 | INTA11/ 1 |
| 113 | 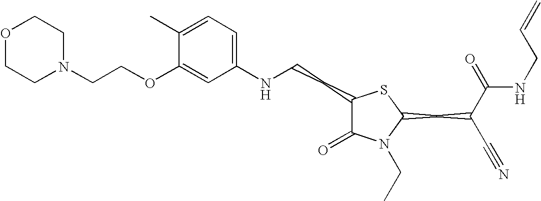 2-Cyano-N-(2-difluoro-ethyl)-2-[3- ethyl-5-[1-[4-(-3-hydroxy-2- piperidin-1-yl-ethyl)-phenylamino]- meth-(E/Z)-ylidene]-4-oxo- thiazolidin-(2-(E or Z))-ylidene]- acetamide | ¹H-NMR (CDCl₃, 300 MHz) δ = 1.39 (m, 3H); 2.17 (s, 3H); 2.58 (m, 4H); 2.86 (m, 2H); 3.72 (m, 4H); 3.95 (m, 2H); 4.07 (m, 2H); 4.36 (m, 2H); 5.20 (m, 2H); 5.86 (m, 1H); 6.27 (m, 1H); 6.50 (d, 1H); 6.58 (m, 1H); 7.08 (d, 1H); 7.56 (d,1H); 10.45 (d, 1H). | MW: 497.62 MS (ESI) [M + 1]⁺: 498 | INTA11/ 1 |
| 114 | 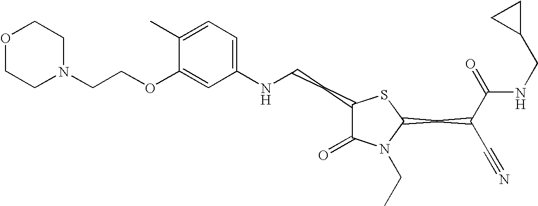 2-Cyano-N-cyclopropylmethyl-2-[3- ethyl-5-[1-[4-methyl-3-(2- morpholin-4-yl-ethoxy)- phenylamino]-meth-(E/Z)-ylidene]- 4-oxo-thiazolidin-(2-(E or Z))- ylidene]-acetamide | ¹H-NMR (CDCl₃, 300 MHz) (selected peaks) δ = 0.19 (m, 2H); 0.50 (m, 2H); 0.99 (m, 1H); 1.38 (m, 3H); 2.12 (s, 3H); 2.60 (m, 4H); 3.17 (m, 2H); 3.70 (m, 4H); 4.09 (m, 2H); 4.32 (m, 2H); 6.30 (m, 1H); 6.60 (m, 2H); 7.05 (m, 1H); 7.55 (d, 1H); 10.42 (d, 1H). | MW: 511.64 MS (ESI) [M + 1]⁺: 512 | INTA11/ 1 |
| 115 | 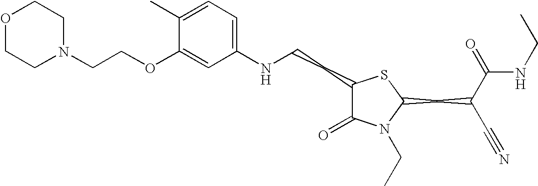 2-Cyano-N-ethyl-2-[3-ethyl-5-[1-[4- methyl-3-(2-morpholin-4-yl-ethoxy)- phenylamino]-meth-(E/Z)-ylidene]- 4-oxo-thiazolidin-(2-(E or Z))- ylidene]-acetamide | ¹H-NMR (DMSO- d6, 300 MHz) (selected peaks) δ = 1.10 (m, 3H); 1.22 (m, 3H); 2.11 (s, 3H); 2.73 (m, 2H); 3.21 (m, 2H); 3.60 (m, 4H); 4.11 (m, 2H); 4.21 (m, 2H); 6.78 (dd, 1H); 6.91 (d, 1H); 7.08 (d, 1H); 7.68 (m, 1H); 8.11 (d, 1H); 10.16 (d, 1H). | MW: 485.61 MS (ESI) [M + 1]⁺: 486 | INTA11/ 1 |
| 116 | 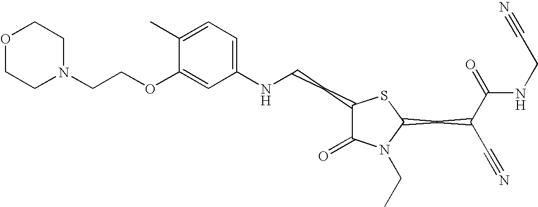 2-Cyano-N-cyanomethyl-2-[3-ethyl- 5-[1-[4-methyl-3-(2-morpholin-4-yl- ethoxy)-phenylamino]-meth-(E/Z)- ylidene]-4-oxo-thiazolidin-(2-(E or Z))-ylidene]-acetamide | ¹H-NMR (DMSO- d6, 300 MHz) (selected peaks) δ = 1.26 (m, 3H); 2.18 (s, 3H); 3.60 (m, 2H); 3.92 (m, 4H); 4.11 (s, 2H); 4.28 (m, 2H); 4.49 (m, 2H); 6.88 (dd, 1H); 6.97 (s, 1H); 7.13 (d, 1H); 8.21 (d, 1H); 10.43 (d, 1H); 11.11 (s, 1H). | MW: 496.59 MS (ESI) [M + 1]⁺: 497 | INTA11/ 1 |
| 117 | 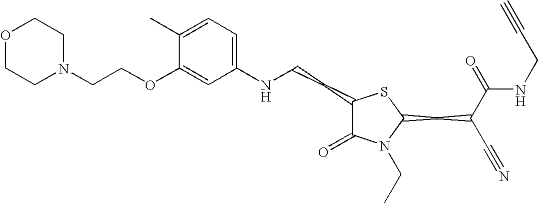 2-Cyan-2-[3-ethyl-5-[1-[4-methyl- 3-(2-morpholin-4-yl-ethoxy)- phenylamino]-meth-(E/Z)-ylidene]- 4-oxo-thiazolidin-(2-(E or Z))- ylidene]-N-prop-2-ynyl-acetamide | ¹H-NMR (CDCl₃, 300 MHz) δ = 1.40 (m, 3H); 2.19 (s, 3H); 2.28 (s, 1H); 2.65 (m, 4H); 2.88 (m, 2H); 3.72 (m, 4H); 4.15 (m, 4H); 4.37 (m, 2H); 6.36 (m, 1H); 6.50 (d, 1H); 6.11 (dd, 1H); 7.09 (d, 1H); 7.53 (d, 1H); 10.48 (d, 1H). | MW: 495.60 MS (ESI) [M + 1]⁺: 496 | INTA11/ 1 |
| 118 | 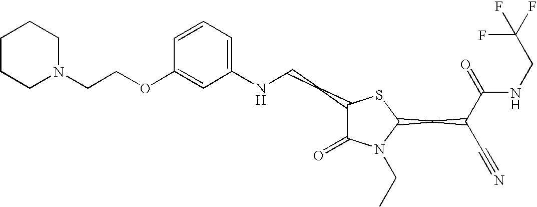 2-Cyano-2-[3-ethyl-4-oxo-5-[1-[3- (2-piperidin-1-yl-ethoxy)- phenylamino]-meth-(E/Z)-ylidene]- thiazolidin-(2-(E or Z))-ylidene]-N- (2,2,2-trifluoro-ethyl)-acetamide | ¹H-NMR (DMSO- d6, 200 MHz) (selected peaks) δ = 1.28 (m, 3H); 1.38 (m, 2H); 1.50 (m, 4H); 2.40 (m, 4H); 2.68 (m, 2H); 3.92 (m, 2H); 4.03 (m, 2H); 4.21 (m, 2H); 6.63 (dd, 1H); 6.90 (m, 2H); 7.24 (m, 1H); 8.20 (m, 2H); 10.30 (s, 1H). | MW: 523.28 MS (ESI) [M + 1]⁺: 524 | INTA12/ 1 |
| 119 | 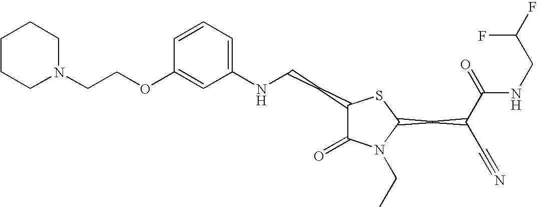 2-Cyano-N-(2,2-difluoro-ethyl)-2-[3- ethyl-4-oxo-5-[1-[3-(2-piperidin-1- yl-ethoxy)-phenylamino]-meth- (E/Z)-ylidene]-thiazolidin-(2-(E or Z))-ylidene]-acetamide | ¹H-NMR (DMSO- d6, 300 MHz) (selected peaks) δ = 1.29 (m, 3H); 1.38 (m, 2H); 1.49 (m, 4H); 2.42 (m, 4H); 2.68 (m, 2H); 3.57 (m, 2H); 4.10 (m, 2H); 4.23 (m, 2H); 6.08 (tt, 1H); 6.62 (dd, 1H); 6.98 (m, 2H); 7.22 (m, 1H); 7.98 (m, 1H); 8.12 (s,1H); 10.27 (s, 1H). | MW: 505.59 MS (ESI) [M + 1]⁺: 506 | INTA12/ 1 |
| 120 | 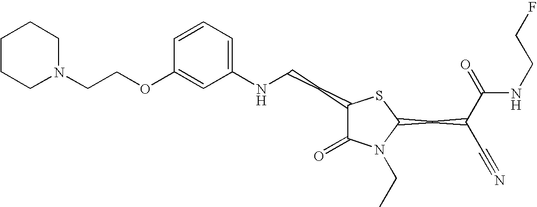 2-Cyano-2-[3-ethyl-4-oxo-5-[1-[3- (2-piperidin-1-yl-ethoxy)- phenylamino]-meth-(E/Z)-ylidene]- thiazolidin-(2-(E or Z))-ylidene]-N- (2-fluoro-ethyl)-acetamide | ¹H-NMR (DMSO- d6, 300 MHz) (selected peaks) δ = 1.28 (m, 3H); 1.38 (m, 2H); 1.50 (m, 4H); 2.42 (m, 4H); 2.68 (m, 2H); 3.47 (m, 1H); 3.54 (m, 1H); 4.09 (m, 2H); 4.21 (m, 2H); 4.42 (m, 1H); 4.59 (m, 1H); 6.63 (dd, 1H); 6.89 (m, 2H); 7.24 (m, 1H);7.82 (m, 1H); 8.10 (s, 1H); 10.22 (s, 1H). | MW: 487.60 MS (ES) [M + 1]⁺: 488 | INTA12/ 1 |
| 121 | 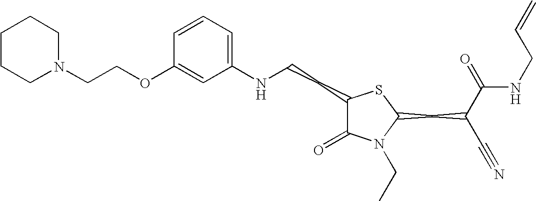 N-Allyl-2-cyano-2-[3-ethyl-4-oxo-5- [1-[3-(2-piperidin-1-yl-ethoxy)- phenylamino]-meth-(E/Z)-ylidene]- thiazolidin-(2-(E or Z))-ylidene]- acetamide | ¹H-NMR (DMSO- d6, 300 MHz) (selected peaks) δ = 1.28 (m, 3H); 1.39 (m, 2H); 1.50 (m, 4H); 2.47 (m, 4H); 2.68 (m, 2H); 3.80 (m, 2H); 4.09 (m, 2H); 4.25 (m, 2H); 5.09 (dd, 1H); 5.12 (dd, 1H); 5.85 (m, 1H); 6.62 (dd, 1H); 6.88 (m, 2H); 7.22 (m, 1H); 7.85 (m,1H); 8.10 (s, 1H); 10.19 (s, 1H). | MW: 481.62 MS (ESI) [M + 1]⁺: 482 | INTA12/ 1 |
| 122 | 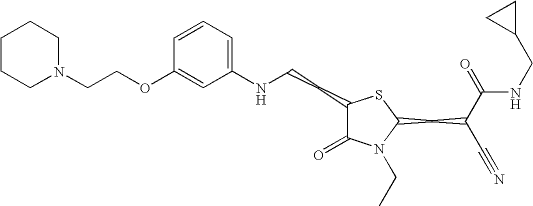 2-Cyano-N-cyclopropylmethyl-2-[3- ethyl-4-oxo-5-[1-[3-(2-piperidin-1- yl-ethoxy)-phenylamino]-meth- (E/Z)-ylidene]-thiazolidin-(2-(E or Z))-ylidene]-acetamide | ¹H-NMR (DMSO- d6, 300 MHz) (selected peaks) δ = 0.21 (m, 2H); 0.40 (m, 2H); 1.00 (m, 1H); 1.22 (m, 3H); 1.38 (m, 2H); 1.50 (m, 4H); 2.41 (m, 4H); 2.62 (m, 2H); 3.04 (m, 2H); 4.09 (m, 2H); 4.21 (m, 2H); 6.63 (dd, 1H); 6.89 (m, 2H); 7.22 (m, 1H);7.77 (m, 1H); 8.09 (s, 1H); 10.20 (s, 1H). | MW: 495.64 MS (ESI) [M + 1]⁺: 496 | INTA12/ 1 |
| 123 | 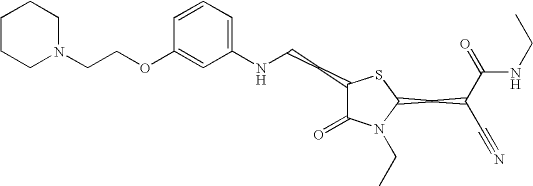 2-Cyano-N-ethyl-2-[3-ethyl--oxo- 5-[1-[3-(2-piperidin-1-yl-ethoxy)- phenylamino]-meth-(E/Z)-ylidene]- thiazolidin-(2-(E or Z))-ylidene]- acetamide | ¹H-NMR (DMSO- d6, 300 MHz) (selected peaks) δ = 1.09 (m, 3H); 1.23 (m, 3H); 1.39 (m, 2H); 1.49 (m, 4H); 2.41 (m, 4H); 2.67 (m, 2H); 3.21 (m, 2H); 4.09 (m, 2H); 4.20 (m, 2H); 6.62 (dd, 1H); 6.87 (m, 2H); 7.21 (m, 1H); 7.70 (m, 1H); 8.10 (s, 1H); 10.18 (s,1H). | MW: 469.61 MS (ESI) [M + 1]⁺: 470 | INTA12/ 1 |
| 124 | 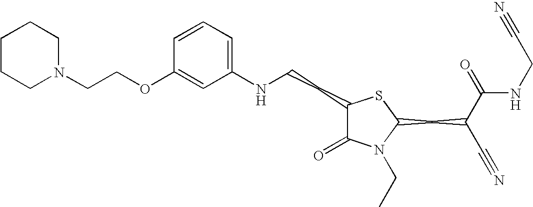 2-Cyano-N-cyanomethyl-2-[3-ethyl- 4-oxo-5-[1-[3-(2-piperidin-1-yl- ethoxy)-phenylamino]-meth-(E/Z)- ylidene]-thiazolidin-(2-(E or Z))- ylidene]-acetamide | ¹H-NMR (DMSO- d6, 300 MHz) (selected peaks) δ = 1.28 (m, 3H); 1.38 (m, 2H); 1.49 (m, 4H); 2.44 (m, 4H); 2.67 (m, 2H); 4.08 (m, 2H); 4.15 (d, 2H); 4.21 (m, 2H); 6.64 (dd, 1H); 6.90 (m, 2H); 7.22 (m, 1H); 8.17 (s, 1H); 8.38 (m, 1H); 10.31 (s, 1H). | MW: 480.59 MS (ESI) [M + 1]⁺: 481 | INTA12/ 1 |
| 125 | 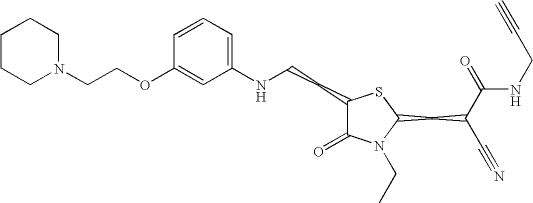 2-Cyano-2-[3-ethyl-4-oxo-5-[1-[3- (2-piperidin-1-yl-ethoxy)- phenylamino]-meth-(E/Z)-ylidene]- thiazolidin-(2-(E or Z))-ylidene]-N- prop-2-ynyl-acetamide | ¹H-NMR (DMSO- d6, 300 MHz) (selected peaks) δ = 1.25 (m, 3H); 1.37 (m, 2H); 1.47 (m, 4H); 2.68 (m, 2H); 3.07 (m, 1H); 3.91 (m, 2H); 4.05 (m, 2H); 4.20 (m, 2H); 6.64 (dd, 1H); 6.89 (m, 2H); 7.24 (m, 1H); 8.11 (m, 2H); 10.27 (s, 1H). | MW: 479.60 MS (ESI) [M + 1]⁺: 480 | INTA12/ 1 |
| 126 | 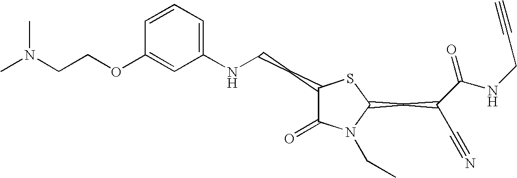 2-Cyano-2-[5-[1-[3-(2- dimethylamino-ethoxy)- phenylamino]-meth-(E/Z)-ylidene]- 3-ethyl-4-oxo-thiazolidin-(2Z or E)- ylidene]-N-prop-2-ynyl-acetamide | ¹H-NMR (CDCl₃, 300 MHz) δ = 1.40 (m, 3H); 2.25 (m, 1H); 2.34 (s, 6H); 2.71 (m, 2H); 4.03 (m, 2H); 4.11 (m, 2H); 4.38 (m, 2H); 6.39 (m, 1H); 6.62 (dd, 1H); 6.69 (m, 1H); 7.21 (d, 1H); 7.56 (s, 1H); 10.48 (s, 1H). | MW: 439.54 MS (ESI) [M + 1]⁺: 440 | INTA13/ 1 |
| 127 | 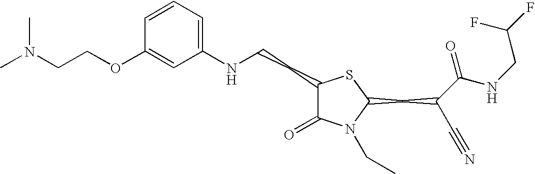 2-Cyano-N-(2,2-difluoro-ethyl)-2-[5- [1-[3-(2-dimethylamino-ethoxy)- phenylamino]-meth-(E/Z)-ylidene]- 3-ethyl-4-oxo-thiazolidin-(2-(E or Z))-ylidene]-acetamide | ¹H-NMR (DMSO- d6, 300 MHz) (selected peaks) δ = 1.21 (m, 3H); 2.28 (s, 6H); 2.63 (m, 2H); 3.58 (m, 2H); 4.05 (m, 2H); 4.25 (m, 2H); 6.08 (tt, 1H); 6.62 (dd, 1H); 6.88 (m, 2H); 7.22 (m, 1H); 7.99 (m, 1H); 8.13 (s, 1H); 10.29 (s, 1H). | MW: 465.52 MS (ESI) [M + 1]⁺: 466 | INTA13/ 1 |
| 128 | 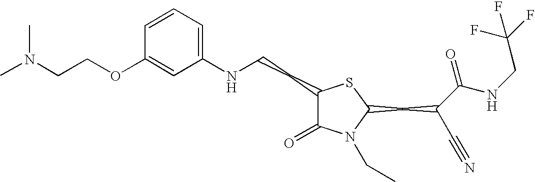 2-Cyano-2-[5-[1-[3-(2- dimethylamino-ethoxy)- phenylamino]-meth-(E/Z)-ylidene]- 3-ethyl-4-oxo-thiazolidin-(2Z or E)- ylidene]-N-(2,2,2-trifluoro-ethyl)- acetamide | ¹H-NMR (DSMO- d6, 300 MHz) (selected peask) δ = 1.22 (m, 3H); 2.25 (s, 6H); 2.67 (m, 2H); 3.96 (m, 2H); 4.09 (m, 2H); 4.26 (m, 2H); 6.63 (dd, 1H); 6.90 (m, 2H); 7.23 (m, 1H); 8.13 (s, 1H); 8.22 (m, 1H); 10.30 (s, 1H). | MW: 483.51 MS (ESI) [M + 1]⁺: 486 | INTA13/ 1 |
| 129 | 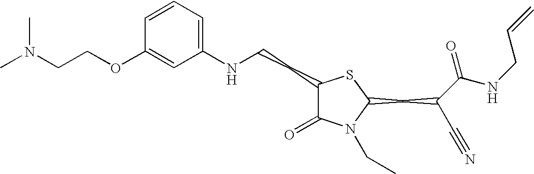 N-Allyl-2-cyano-2-[5-[1-[3-(2- dimethylamino-ethoxy)- phenylamino]-meth-(E/Z)-ylidene]- 3-ethyl-4-oxo-thiazolidin-(2-(E or Z))-ylidene]-acetamide | ¹H-NMR (DSMO- d6, 300 MHz) (selected peaks) δ = 1.23 (m, 3H); 2.22 (s, 6H); 2.61 (m, 2H); 3.79 (m, 2H); 4.08 (m, 2H); 4.22 (m, 2H); 5.08 (dd, 1H); 5.12 (dd, 1H); 5.81 (m, 1H); 6.63 (dd, 1H); 6.89 (m, 2H); 7.22 (m, 1H); 7.83 (m, 1H); 8.10 (s, 1H); 10.20 (s, 1H). | MW: 441.55 MS (ESI) [M + 1]⁺: 442 | INTA13/ 1 |
| 130 | 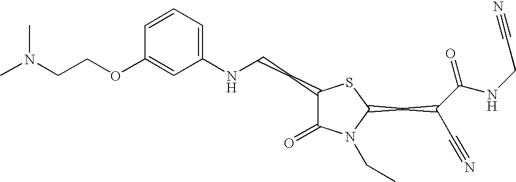 2-Cyano-N-cyanomethyl-2-[5-[1-[3- (2-dimethylamino-ethoxy)- phenylamino]-meth-(E/Z)-ylidene]- 3-ethyl-4-oxo-thiazolidin-(2-(E or Z))-ylidene]-acetamide | ¹H-NMR (DMSO- d6, 300 MHz) (selected peaks) δ = 1.27 (m, 3H); 2.26 (s, 6H); 2.62 (m, 2H); 4.07 (m, 2H); 4.16 (m, 2H); 4.22 (m, 2H); 6.63 (dd, 1H); 6.90 (m, 2H); 7.25 (m, 1H); 8.18 (s, 1H); 8.37 (m, 1H); 10.33 (s, 1H). | MW: 440.53 MS (ESI) [M + 1]⁺: 441 | INTA13/ 1 |
| 131 | 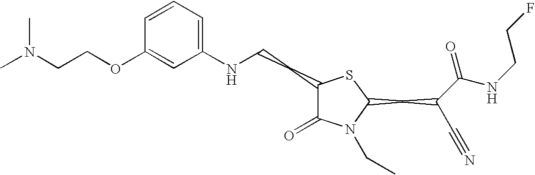 2-Cyano-2-[5-[1-[3-(2- dimethylamino-ethoxy)- phenylamino]-meth-(E/Z)-ylidene]- 3-ethyl-4-oxo-thiazolidin-(2Z or E)- ylidene]-N-(2-fluoro-ethyl)- acetamide | ¹H-NMR (DMSO- d6, 300 MHz) (selected peaks) δ = 1.26 (m, 3H); 2.24 (s, 6H); 2.62 (m, 2H); 3.45 (m, 1H); 3.51 (m, 1H); 4.09 (m, 2H); 4.21 (m, 2H); 4.42 (m, 2H); 4.58 (m, 2H); 6.62 (dd, 1H); 6.89 (m, 2H); 7.22 (m, 1H); 7.81 (m, 1H); 8.11 (s, 1H);10.23 (s, 1H). | MW: 447.53 MS (ESI) [M + 1]⁺: 448 | INTA13/ 1 |
| 132 | 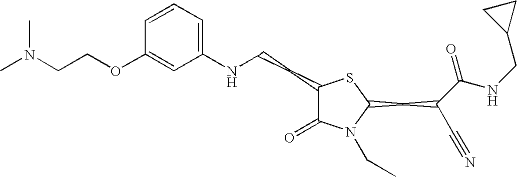 2-Cyano-N-cyclopropylmethyl-2-[5- [1-[3-(2-dimethylamino-ethoxy)- phenylamino]-meth-(E/Z)-ylidene]- 3-ethyl-4-oxo-thiazolidin-(2-(E or Z))-ylidene]-acetamide | ¹H-NMR (DMSO- d6, 300 MHz) (selected peaks) δ = 0.21 (m, 2H); 0.41 (m, 2H); 1.00 (m, 1H); 1.25 (m, 3H); 2.21 (s, 6H); 2.61 (m, 2H); 3.04 (m, 2H); 4.07 (m, 2H); 4.21 (m, 2H); 6.62 (dd, 1H); 6.88 (m, 2H); 7.22 (m, 1H); 7.77 (m, 1H); 8.09 (s, 1H);10.19 (s, 1H). | MW: 455.58 MS (ESI) [M + 1]⁺: 456 | INTA13/ 1 |
| 133 | 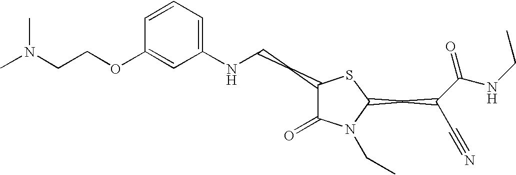 2-Cyano-2-[5-[1-[3-(2- dimethylamino-ethoxy)- phenylamino]-meth-(E/Z)-ylidene]- 3-ethyl-4-oxo-thiazolidin-(2Z or E)- ylidene]-N-ethyl-acetamide | ¹H-NMR (DMSO- d6, 300 MHz) (selected peaks) δ = 1.05 (m, 3H); 1.22 (m, 3H); 2.22 (s, 6H); 2.65 (m, 2H); 3.21 (m, 2H); 4.03 (m, 2H); 4.20 (m, 2H); 6.63 (dd, 1H); 6.89 (m, 2H); 7.21 (m, 1H); 7.70 (m, 1H); 8.10 (s, 1H); 10.20 (s, 1H). | MW: 429.54 MS (ESI) [M + 1]⁺: 430 | INTA13/ 1 |
| 134 | 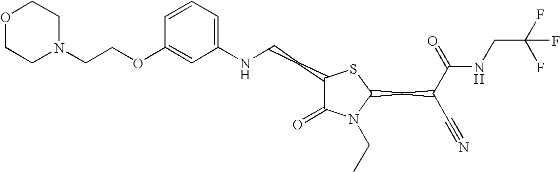 2-Cyano-2-[3-ethyl-5-[1-[3-(2- morpholin-4-yl-ethoxy)- phenylamino]-meth(E/Z ylidene]-4- oxo-thiazolidin-(2Z or E)-ylidene]- N-(2,2,2-trifluoro-ethyl)-acetamide | ¹H-NMR (DMSO- d6, 300 MHz) (selected peaks) δ = 1.29 (m, 3H); 2.71 (m, 2H); 3.58 (m, 4H); 3.95 (m, 2H); 4.10 (m, 2H); 4.22 (m, 2H); 6.65 (dd, 1H); 6.90 (m, 2H); 7.22 (m, 1H); 8.20 (m, 2H); 10.31 (d, 1H). | 525.5/ 526 | INTA7/ 1 |
| 135 | 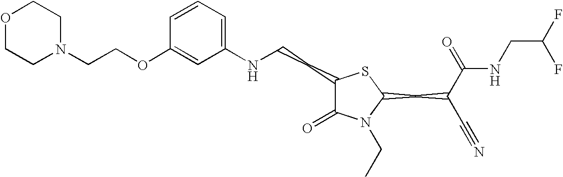 2-Cyano-N-(2,2-difluoro-ethyl)-2-[3- ethyl-5-[1-[3-(2-morpholin-4-yl- ethoxy)-phenylamino]-meth-(E/Z)- ylidene]-4-oxo-thiazolidin-(2Z or E)- ylidene]-acetamide | ¹H-NMR (DMSO- d6, 300 MHz) (selected peaks) δ = 1.25 (m, 3H); 2.70 (m, 2H); 3.60 (m, 6H); 4.11 (m, 2H); 4.22 (m, 2H); 6.08 (tt, 1H); 6.64 (ddm 1H); 6.89 (m, 2H); 7.21 (m, 1H); 7.98 (m, 1H); 8.12 (s, 1H); 10.29 (s, 1H). | 507.5/ 508 | INTA7/ 1 |
| 136 | 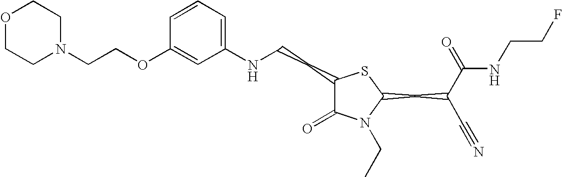 2-Cyano-2-[3-ethyl-5-[1-[3-(2- morpholin-4-yl- ethoxy)phenylamino]-meth-(E/Z)- ylidene]-4-oxo-thiazolidin-(2Z or E)- ylidene]-N-(2-fluoro-ethyl)- acetamide | ¹H-NMR (DMSO- d6, 300 MHz) (selected peaks) δ 1.26 (m, 3H); 2.70 (m, 2H); 3.48 (m, 1H); 3.51 (m, 1H); 3.60 (m, 4H); 4.11 (m, 2H); 4.22 (m, 2H); 4.40 (m, 1H); 4.58 (m, 1H); 6.63 (dd, 1H); 6.89 (m, 2H); 7.21 (m, 1H); 7.81 (m, 1H); 8.11 (d, 1H); 10.26 (d, 1H). | 489.5/ 490 | INTA7/ 1 |
| 137 | 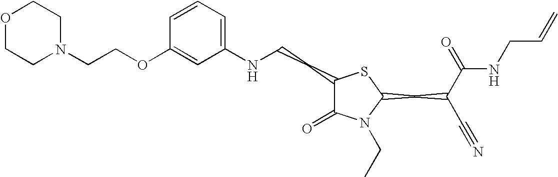 N-Allyl-2-cyano-2-[3-ethyl-5-[1-[3- (2-morpholin-4-yl-ethoxy)- henylamino]-meth-(E/Z)-ylidene]- 4-oxo-thiazolidin-(2Z or E)-ylidene]- acetamide | ¹H-NMR (DMSO- d6, 300 MHz) (selected peaks) δ = 1.27 (m, 3H); 2.70 (m, 2H); 3.58 (m, 4H); 3.80 (m, 2H); 4.11 (m, 2H); 4.22 (m, 2H); 5.08 (dd, 1H); 5.12 (dd, 1H); 5.82 (m, 1H); 6.63 (dd, 1H); 6.88 (m, 2H); 7.21 (m, 1H); 7.87 (m, 1H); 8.10 (s, 1H); 10.20 (s, 1H). | 483.5/ 484 | INTA7/ 1 |
| 138 | 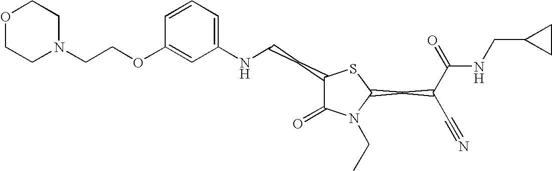 2-Cyano-N-cyclopropylmethyl-2-[3- ethyl-5-[1-[3-(2-morpholin-4-yl- ethoxy)phenylamino]-meth-(E/Z)- ylidene]-4-oxo-thiazolidin-(2Z or E)- ylidene]-acetamide | ¹H-NMR (DMSO- d6, 300 MHz) (selected peaks) δ = 0.21 (m,2 H); 0.42 (m, 2H); 1.02 (m, 1H); 1.27 (m, 3H); 2.70 (m, 2H); 3.03 (m, 2H); 3.61 (m, 4H); 4.10 (m, 2H); 4.27 (m, 2H); 6.66 (dd, 1H); 6.88 (m, 2H); 7.22 (m, 1H); 7.78 (m, 1H); 8.11 (s,1H); 10.19 (s, 1H). | 497.6/ 498 | INTA7/ 1 |
| 139 | 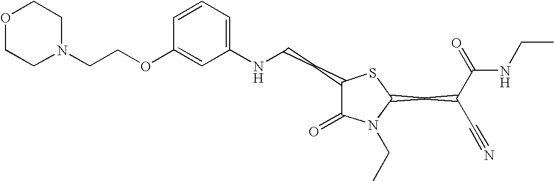 2-Cyano-N-2-[3-ethyl-5-[1-[3- (2-morpholin-4-yl-ethoxy)- henylamino]-meth-(E/Z)-ylidene]- 4-oxo-thiazolidin-(2Z or E)-ylidene]- acetamide | ¹H-NMR (DMSO- d6, 300 MHz) (selected peaks) δ = 1.08 (m, 3H); 1.28 (m, 3H); 2.70 (m, 2H); 3.20 (m, 2H); 3.59 (m, 4H); 4.11 (m, 2H); 4.22 (m, 2H); 6.62 (dd, 1H); 6.97 (m, 2H); 7.23 (m, 1H); 7.72 (m, 1H); 8.10 (s, 1H), 10.20 (s, 1H). | 471.5/ 472 | INTA7/ 1 |
| 140 | 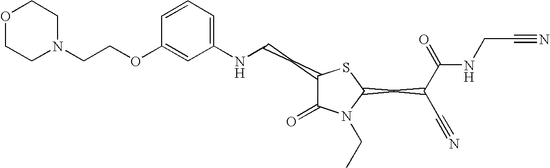 2-Cyano-N-cyanomethyl-2-[3-ethyl- 5-[1-[3-(2-morpholin-4-yl-ethoxy)- phenylamino]-meth-(E/Z)- ylidene]-4-oxo-thiazolidin-(2Z or E)- ylidene]-acetamide | ¹H-NMR (DMSO- d6, 300 MHz) (selected peaks) δ = 1.25 (m, 3H); 2.71 (m, 2H); 3.60 (m, 4H); 4.13 (m, 2H); 4.16 (m, 2H); 4.21 (m, 2H); 6.69 (dd, 1H); 6.90 (m, 2H); 7.21 (m, 1H); 8.18 (d, 1H); 8.37 (m, 1H); 10.32 (d, 1H). | 482.5/ 483 | INTA7/ 1 |
| 141 | 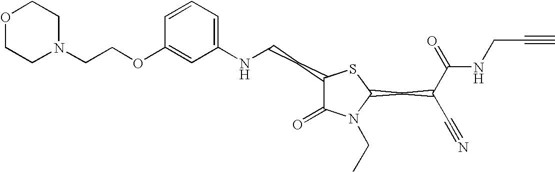 2-Cyano-2-[3-ethyl-5-[1-[3-(2- morpholin-4-yl-ethoxy)- phenylamino]-meth-(E/Z)- ylidene]-4-oxo-thiazolidin-(2Z or E)- ylidene]-N-prop-2-ynyl-acetamide | ¹H-NMR (DMSO- d6, 300 MHz) (selected peaks) δ = 1.25 (m, 3H); 2.71 (m, 2H); 3.09 (m, 1H); 3.58 (m, 4H); 3.93 (m, 2H); 4.10 (m, 2H); 4.22 (m, 2H); 6.63 (dd, 1H); 6.89 (m, 2H); 7.22 (m, 1H); 8.12 (m, 1H); 10.27 (s, 1H). | 481.5/ 482 | INTA7/ 1 |
| 142 | 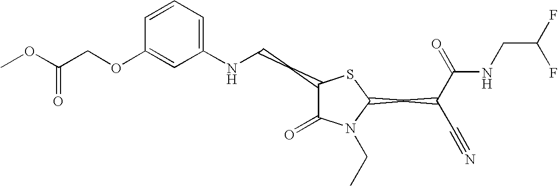 (3-{[2-[1-Cyano-1-(2,2-difluoro- ethylcarbamoyl)-meth-(Z or E)- ylidene]-3-ethyl-4-oxo-thiazolidin- (5E/Z)-ylidenemethyl]-amino}- phenyoxy)-acetic acid methyl ester | ¹H-NMR (DMSO- d6, 300 MHz) (selected peaks) δ 1.18 (m, 3H); 3.48 (m, 2H); 3.69 (s, 3H); 4.19 (m, 2H); 4.50 (s, 2H); 5.98 (tt, 1H); 6.60 (dd, 1H); 6.83 (m, 2H); 7.19 (m, 1H); 8.18 (s, 1H); 8.40 (m, 1H); 10.50 (s, 1H). | 466.4/ 467 | INTA15/ 1 |
| 143 | 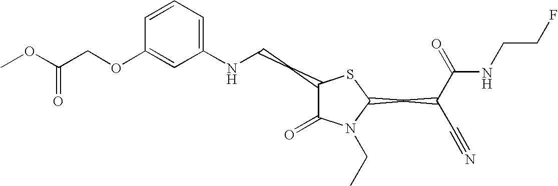 (3-{[2-[1-Cyano-1-(2-fluoro- ethylcarbamoyl)-meth-(Z or E)- ylidene]-3-ethyl-4-oxo-thiazolidin- (5E or Z)-ylidenemethyl]-amino}- phenoxy)-acetic acid methyl ester | ¹H-NMR (DMSO- d6, 300 MHz) (selected peaks) δ 1.25 (m, 3H); 3.50 (m, 1H); 3.78 (s, 3H); 4.23 (m, 2H); 4.40 (m, 1H); 4.57 (m, 3H); 6.70 (dd, 1H); 6.93 (m, 2H); 7.28 (m, 1H); 8.21 (s, 1H); 8.32 (m, 1H); 10.57 (s, 1H). | 448.4/ 449 | INTA15/ 1 |
| 144 | 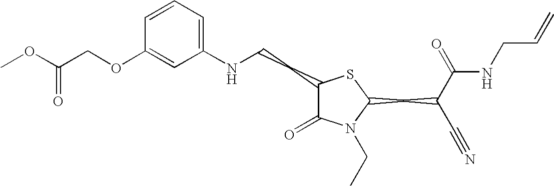 (3-{[2-[1-Allylcarbamoyl-1-cyano- meth-(Z or E)-ylidene]-3-ethyl-4- oxo-thiazolidin-(5E/Z)- ylidenemethyl]-amino}-phenoxy)- acetic acid methyl ester | ¹H-NMR (DMSO- d6, 300 MHz) (selected peaks) δ = 1.24 (m, 3H); 3.79 (m, 5H); 4.27 (m, 2H); 4.58 (s, 2H); 5.07 (dd, 1H); 5.11 (dd, 1H); 5.81 (m, 1H); 6.70 (dd, 1H); 6.92 (m, 2H); 7.28 (m, 1H); 8.22 (s, 1H); 8.30 (m, 1H); 10.58 (s, 1H). | 442.4/ 443 | INTA15/ 1 |
| 145 | 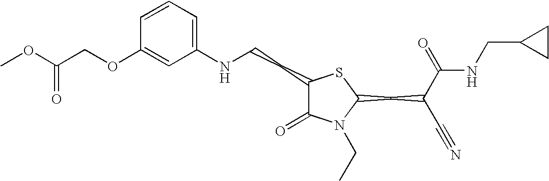 (3-{[2-[1-Cyano-1- cyclopropylmethyl-carbamoyl)-eth- (Z or E)-ylidene]-3-ethyl-4-oxo- thiazlolidin-(5E/Z)-ylidenemethyl]- amino}-phenyoxy)-acetic acid methyl ester | ¹H-NMR (DMSO- d6, 300 Mhz) (selected peaks) δ = 0.19 (m, 2H); 0.39 (m, 2H); 0.45 (m, 1H); 1.21 (m, 3H); 3.02 (m, 2H); 3.79 (s, 3H); 4.25 (m, 2H); 4.51 (s, 2H); 6.70 (dd, 1H); 6.94 (m, 2H); 7.29 (m, 1H); 8.18 (m, 1H); 8.26 (s, 1H); 10.58 (s, 1H). | 456.5/ 457 | INTA15/ 1 |
| 146 | 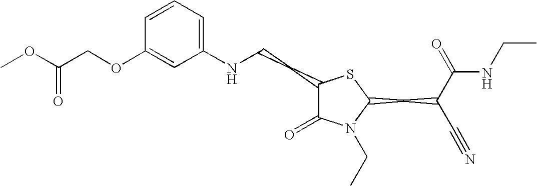 (3-{[2-[1-Cyano-1-ethylcarbamoyl- meth-(E or Z)-ylidene]-3-ethyl-4- oxo-thiazolidin-(5E/Z)- ylidenemethyl]-amino}-phenoxy)- acetic acid methyl ester | ¹H-NMR (DMSO- d6, 300 MHz) (selected peaks) δ = 1.02 (m, 3H); 1.22 (m, 3H); 3.18 (m, 2H); 3.79 (s, 3H); 4.28 (m, 2H); 4.50 (s, 2H); 6.71 (dd, 1H); 6.98 (m, 2H); 7.28 (m, 1H); 8.11 (m, 1H); 8.22 (s, 1H); 10.48 (s, 1H). | 430.4/ 431 | INTA15/ 1 |
| 147 | 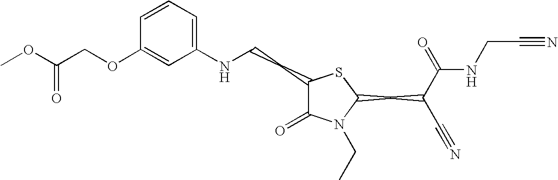 (3-{[2-[1-Cyano-1-(cyanomethyl- carbamoyl)-meth-(Z or E)-ylidene]- 3-ethyl-4-oxo-thiazolidin-(5E or Z)- ylidenemethyl]-amino}-phenoxy)- acetic acid methyl ester | ¹H-NMR (DMSO- d6, 300 Mhz) (selected peaks) δ 1.22 (m, 3H); 3.79 (s, 3H); 4.22 (m, 4H); 4.68 (s, 2H); 6.71 (dd, 1H); 6.95 (m, 2H); 7.30 (m, 1H); 8.22 (s, 1H); 8.86 (m, 1H); 10.53 (s, 1H). | 441.4/ 442 | INTA15/ 1 |
| 148 | 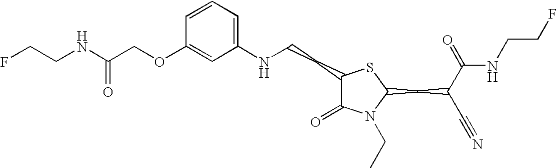 2-Cyano-2-[3-ethyl-5-[1-{3-[(2- fluoro-ethoxycarbamoyl)-methoxy]- phenylamino}-meth-(E/Z)- ylidene]-4-oxo-thiazolidin-(2Z or E)- ylidene]-N-(2-fluoro-ethyl)- acetamide | ¹H-NMR (DMSO- d6, 300 MHz) (selected peaks) δ = 1.24 (m, 3H); 3.50 (m, 4H); 4.24 (m, 2H); 4.40 (m, 2H); 4.57 (m, 4H); 6.68 (dd, 1H); 6.92 (m, 2H); 7.28 (m, 1H); 7.80 (m, 1H); 8.10 (s, 1H); 8.32 (m, 1H); 10.31 (s, 1H). | 479.5/ 480 | INTA15/ 1 |
| 149 | 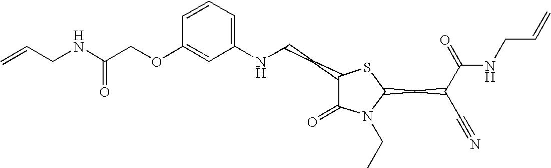 N-Allyl-2-[5-[1-(3- allylcarbamoylmethoxy- phenylamino)-meth-(E/Z)- ylidene]-3-ethyl-4-oxo-thiazolidin- (2Z or E)-ylidene]-2-cyano- acetamide | ¹H-NMR (DMSO- d6, 300 MHz) (selected peaks) δ = 1.28 (m, 3H); 3.78 (m, 4H); 4.26 (m, 2H); 4.54 (s, 2H); 5.10 (m, 4H); 5.81 (m, 2H); 6.69 (dd, 1H); 6.91 (m, 2H); 7.27 (m, 1H); 7.88 (m 1H); 8.10 (d, 1H); 8.30 (m, 1H); 10.28 (d, 1H). | 467.5/ 468 | INTA15/ 1 |
| 150 | 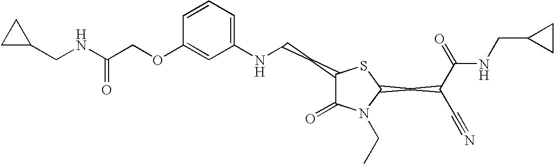 2-Cyano-N-cyclopropylmethyl-2-[5- ]1-{3-[(cyclopropylmethyl- carbamoyl)-methoxy]- phenylamino}-meth-(E/Z)- ylidene]-3-ethyl-4-oxo-thiazolidin- (2Z or E)-ylidene]-acetamide | ¹H-NMR (DMSO- d6, 300 MHz) (selected peaks) δ = 0.20 (m, 4H); 0.39 (m, 4H); 0.97 (m, 2H); 1.27 (m, 3H); 3.04 (m, 4H); 4.21 (m, 2H); 4.50 (s, 2H); 6.68 (dd, 1H); 6.91 (m, 2H); 7.22 (m, 1H); 7.75 (s, 1H); 8.09 (s, 1H); 8.16(m, 1H); 10.27 (s, 1H). | 495.6/ 496 | INTA15/ 1 |
| 151 | 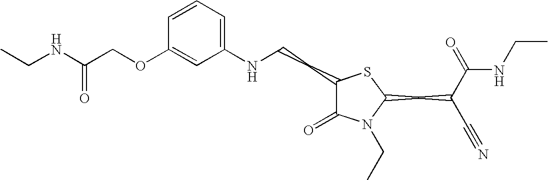 2-Cyano-N-ethyl-2-[3-ethyl-5-[1-(3- ethylcarbamoylmethoxy- phenylamino)-meth-(E/Z)- ylidene]-4-oxo-thiazolidin-(2Z or E)- ylidene]-acetamide | ¹H-NMR (DMSO- d6, 300 MHz) (selected peaks) δ = 1.09 (m, 6H); 1.28 (m, 3H); 3.20 (m, 4H); 4.21 (m, 2H); 4.50 (s, 2H); 6.68 (dd, 1H); 6.92 (m, 2H); 7.27 (m, 1H); 7.70 (m, 1H); 8.10 (d, 1H); 10.26 (d, 1H). | 443.5/ 444 | INTA15/ 1 |
| 152 | 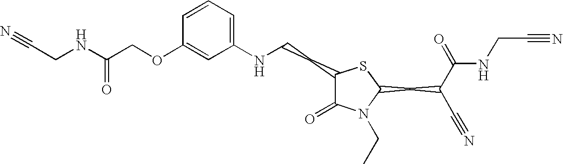 2-Cyano-N-cyanomethyl-2-[5-[1-{3- [(cyanomethyl-carbamoyl)- methoxy]-phenylamino}-meth-(E/ Z)-ylidene]-3-ethyl-4-oxo- thiazolidin-(2Z or E)-ylidene]- acetamide | ¹H-NMR (DMSO- d6, 300 MHz) (selected peaks) δ = 1.29 (m, 3H); 4.10 (m, 6H); 4.57 (s, 2H); 6.60 (dd, 1H); 6.88 (m, 2H); 7.19 (m, 1H); 8.08 (s, 1H); 8.26 (s, 1H); 8.78 (m, 1H); 10.33 (s, 1H). | 465.4/ 466 | INTA15/ 1 |
| 153 | 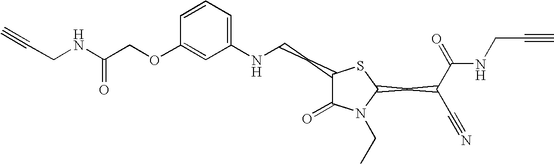 2-Cyano-2-[3-ethyl-4-oxo-5-[1-(3- prop-2-ynylcarbamoylmethoxy- phenylamino)-meth-(E/Z)- ylidene]-thiazolidin-(2Z or E)- ylidene]-N-prop-2-ynyl-acetamide | ¹H-NMR (DMSO- d6, 300 MHz) (selected peaks) δ = 1.28 (m, 3H); 3.08 (m, 1H); 3.13 (m, 1H); 3.92 (m, 4H); 4.22 (m, 2H); 4.55 (s, 2H); 6.69 (dd, 1H); 6.92 (m,2 H); 7.28 (m, 1H); 8.11 (d, s, 1H); 8.60 (m, 1H); 10.31 (s, 1H). | 463.5/ 464 | INTA15/ 1 |
| 154 | 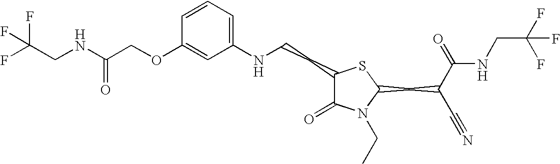 2-Cyano-2-[3-ethyl-4-oxo-5-[1-{3- [(2,2,2-trifluoro-ethylcarbamoyl)- methoxy]-phenylamino}-meth-(E/ Z)-ylidene]-thiazolidin-(2Z or E)- ylidene]-N-(2,2,2-trifluoro-ethyl)- acetamide | ¹H-NMR (DMSO- d6,300 MHz) (selected peaks) δ = 1.28 (m, 3H); 3.97 (m, 4H); 4.22 (m, 2H); 4.63 (s, 2H); 6.69 (dd, 1H); 6.93 (m, 2H); 7.28 (m, 1H); 8.20 (m, 2H); 8.78 (m,1 H); 10.40 (s, 1H). | 551.4/ 552 | INTA15/ 1 |
| 155 | 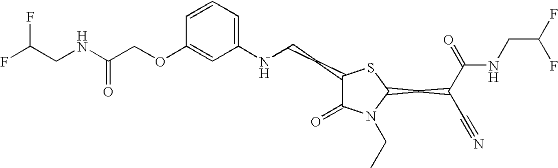 2-Cyano-N-(2,2-difluoro-ethyl)-2-[5- [1-{3-[(2,2-difluoro- ethylcarbamoyl)-methoxy]- phenylamino}-meth-(E/Z)- ylidene]-3-ethyl-4-oxo-thiazolidin- (2Z or E)-ylidene]-acetamide | ¹H-NMR (DMSO- d6, 300 MHz) (selected peaks) δ = 1.20 (m, 3H); 3.47 (m, 4H); 4.15 (m, 2H); 4.50 (s, 2H); 5.97 (tm, 2H); 6.60 (dd, 1H); 6.82 (m, 2H); 7.19 (m, 1H); 7.88 (s, 1H); 8.03 (s, 1H); 8.40 (m, 1H); 10.26 (s, 1H). | 515.4/ 516 | INTA15/ 1 |
| 156 | 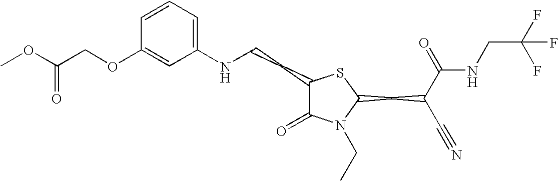 (3-{[2-[1-Cyano-1-(2,2,2-trifluoro- ethylcarbamoyl)-meth-(Z or E)- ylidene]-3-ethyl-4-oxo-thiazolidin- (5E/Z)-ylidenemethyl]-amino}- phenoxy)-acetic acid methyl ester | ¹H-NMR (DMSO- d6, 300 MHz) (selected peaks) δ = 1.19 (m, 3H); 3.68 (s, 3H); 3.89 (m, 2H); 4.18 (m, 2H); 4.57 (s, 2H); 6.60 (d, 1H); 6.83 (m, 2H); 7.69 (m, 1H); 8.15 (s, 1H); 8.70 (m, 1H); 10.50 (s, 1H). | 484.4/ 485 | INTA15/ 1 |
| 157 | 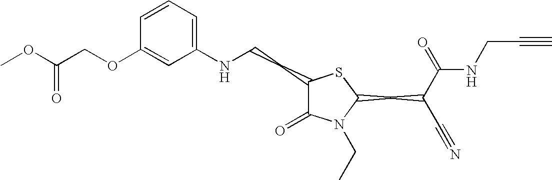 (3-{[2-[1-Cyano-1-prop-2- ynylcarbamoyl-meth-(Z or E)- ylidene]-3-ethyl-4-oxo-thiazolidin- (5E/Z)-ylidenemethyl]-amino}- phenoxy)-acetic acid methyl ester | ¹H-NMR (DMSO- d6,300 MHz) (selected peaks) δ = 1.27 (m, 3H), 3.11 (m, 1H); 3.79 (s, 3H); 3.93 (m, 2H); 4.25 (m, 2H); 4.58 (s, 2H); 6.71 (dd, 1H); 6.97 (m, 2H); 7.29 (m, 1H); 8.22 (s, 1H); 8.60 (m, 1H); 10.57 (s, 1H). | 440.4/ 441 | INTA15/ 1 |
| 158 | 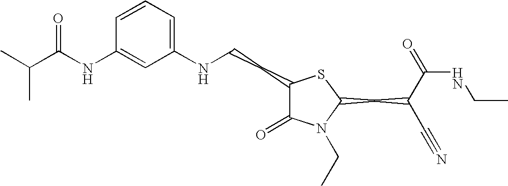 2-Cyano-N-ethyl-2-[3-ethyl-5-[1-(3- isobutyrylamino-phenylamino)-eth- (E/Z)-ylidene]-4-oxo-thiazolidin-(2- (E or Z))-ylidene]-acetamide | (DMSO-d6, stored via K₂CO₃, primary isomer): δ = 1.05-1.11 (m, 9H); 1.24 (t, 3H); 2.54-2.61 (m, 1H); 3.14-3.23 (m, 2H); 4.21 (d, 2H); 6.91 (d, 1H); 7.18-7.24 (m, 2H); 7.67-7.69 (m, 2H); 7.96 (d, 2H); 9.87 (s, 1H); 10.36 (d, 1H) ppm. | 427.53/ 428 | INTA16/ 1 |
| 159 | 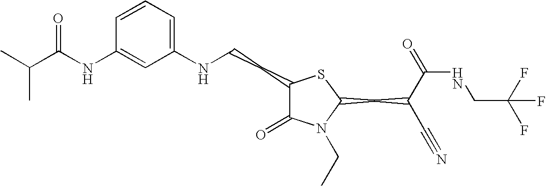 2-Cyano-2-{3-ethyl-5-[1-(3- isobutyryl-amino-phenylamino)- meth-(E/Z)-ylidene]-4-oxo- thiazolidin-(2-(E or Z))-ylidene}-N- (2,2,2-trifluoro-ethyl)-acetamide | (DMSO-d6, stored via K₂CO₃, primary isomer): δ = 1.11 (d, 6H); 1.25 (t, 3H); 2.58 (m, 1H); 3.94 (m, 2H); 4.23 (q, 2H); 6.92 (d, 1H); 7.18-7.25 (m, 2H); 7.70 (s, 1H); 8.01 (d, 1H); 8.19 (t, 1H); 9.87 (s, 1H); 10.47 (d, 1H) ppm. | 481.50/ 482 | INTA16/ 1 |
| 160 | 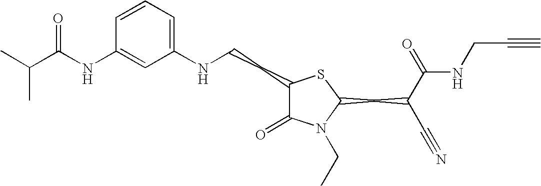 2-Cyano-2-{3-ethyl-5-[1-(3- isobutyryl-amino-phenylamino)- meth-(E/Z)-ylidene]-4-oxo- thiazolidin-(2-(E or Z))-ylidene}-N- prop-2-ynyl-acetamide | (DMSO-d6, stored via K₂CO₃, primary isomer): δ = 1.11 (d, 6H); 1.24 (t, 3H); 2.53-2.63 (m, 1H); 3.05 (t, 1H); 3.92 (dd, 2H); 4.22 (q, 2H); 6.92 (d, 1H); 7.18-7.25 (m, 2H); 7.70 (s, 1H); 8.80 (d, 1H); 8.07 (t, 1H); 9.86 (s, 1H); 10.42 (d, 1H) ppm. | 437.52/ 438 | INTA16/ 1 |
| 161 | 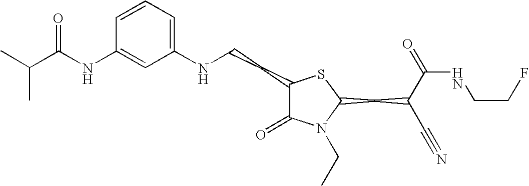 2-Cyano-2-{3-ethyl-5-[1-(3- isobutyryl-amino-phenylamino)- meth-(E/Z)-ylidene]-4-oxo- thiazolidin-(2-(E or Z))-ylidene}-N- (2-fluoro-ethyl)-acetamide | (DMSO-d6, stored via K₂CO₃, primary isomer): δ = 1.11 (d,6H); 1.24 (t, 3H); 2.53-2.63 (m, 1H); 3.43-3.53 (2q, 1H); 4.22 (t, 1H); 4.40-4.55 (2t, 1H); 6.92 (d, 1H); 7.18-7.25 (m, 2H); 7.69 (s, 1H); 7.74 (t, 1H); 7.98 (1H); 9.86 (s, 1H); 10.40 (1H)ppm. | 445.52/ 446 | INTA16/ 1 |
| 162 | 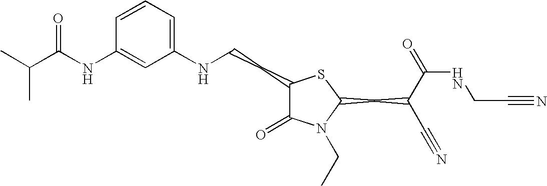 2-Cyano-N-cyanomethyl-2-{3-ethyl- 5-[1-(3-isobutyrylamino- phenylamino)-meth-(E/Z)-ylidene]- 4-oxo-thiazolidin-(2-(E or Z))- ylidene}-acetamide | (DMSO-d6, stored via K₂CO₃, primary isomer): δ = 1.11 (d, 6H); 1.25 (t, 3H); 2.53-2.63 (m, 1H); 4.15 (d, 2H); 4.22 (q, 2H); 6.94 (d, 1H); 7.19-7.26 (m, 2H); 7.72 (s, 1H); 7.74 (t, 1H); 8.03 (1H); 9.88 (s, 1H); 10.50 (1H) ppm. | 438.51/ 439 | INTA16/ 1 |
| 163 | 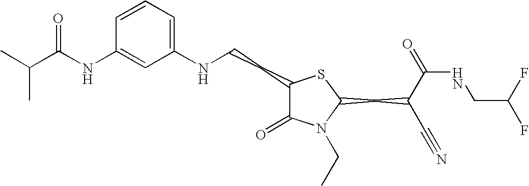 2-Cyano-N-(2,2-difluoro-ethyl)-2- {3-ethyl-5-[1-(3-isobutyrylamino- phenylamino)-meth-(E/Z)-ylidene]- 4-oxo-thiazolidin-(2-(E or Z))- ylidene}-acetamide | (DMSO-d6, stored via K₂CO₃, primary isomer): δ = 1.10 (d, 6H); 1.24 (t, 3H); 2.54-2.61 (m, 1H); 3.52-3.62 (m, 2H); 4.22 (q, 2H); 5.88-6.18 (3t, 1H); 6.91 (d, 1H); 7.17-7.24 (m, 2H); 7.69 (s, 1H); 7.91 (t, 1H); 7.98 (d, 1H); 9.85 (s, 1H); 10.45(1H) ppm. | 463.51/ 464 | INTA16/ 1 |
| 164 | 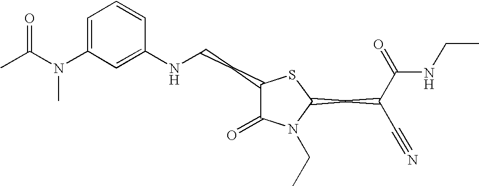 2-{5-[1-[3-(Acetyl-methyl-amino)- phenyl-amino]-meth-(E/Z)-ylidene]- 3-ethyl-4-oxo-thiazolidin-(2-(E or Z))-ylidene}-2-cyano-N-ethyl- acetamide | (DMSO-d6, stored via K₂CO₃, primary isomer): δ = 1.06 (t, 3H); 1.25 (t, 3H); 1.80 (3H); 3.10-3.23 (m, 5H); 4.21 (q, 2H); 6.96 (d, 1H); 7.22 (d, 1H); 7.30-7.37 (m, 2H); 7.68 (t, 1H); 8.11 (t, 1H); 10.40 (1H) ppm. | 413.50/ 414 | INTA17/ 1 |
| 165 | 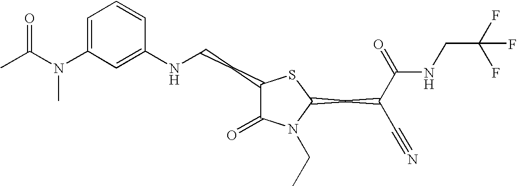 2-{5-[1-[3-(Acetyl-methyl-amino)- phenylamino]-meth-(E/Z)-ylidene]- 3-ethyl-4-oxo-thiazolidin-(2-(E or Z))-ylidene}-2-cyano-N-(2,2,2- trifluoro-ethyl)-acetamide | (DMSO-d6, stored via K₂CO₃, primary isomer): δ = 1.25 (t, 3H); 1.80 (3H); 3.14 (3H); 3.89-3.98 (m, 2H); 4.23 (q, 2H); 6.97 (d, 1H); 7.22 (m, 1H); 7.28-7.36 (m, 2H); 8.15-8.21 (m, 2H); 10.34 (d, 1H) ppm. | 467.47/ 468 | INTA17/ 1 |
| 166 | 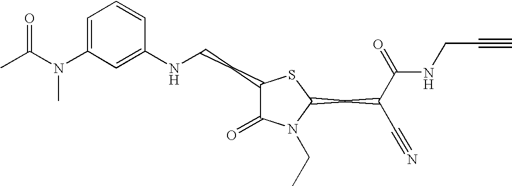 2-{5-[1-[3-(Acdtyl-methyl-amino)- phenyl-amino]-meth-(E/Z)-ylidene]- 3-ethyl-4-oxo-thiazolidin-(2-(E or Z))-ylidene}-2-cyano-N-prop-2-ynyl- acetamide | (DMSO-d6, stored via K₂CO₃, primary isomer): δ = 1.24 (t, 3H); 1.80 (3H); 3.05 (t, 1H); 3.15 (3H); 3.90-3.92 (m, 2H); 4.18 (q, 2H); 6.98 (d, 1H); 7.24 (m, 1H); 7.33-7.38 (m, 2H); 8.10 (t, 1H); 8.16 (d, 1H); 10.32 (d, 1H) ppm. | 423.50/ 424 | INTA17/ 1 |
| 167 | 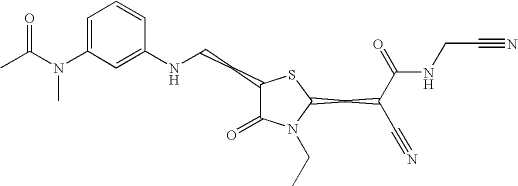 2-{5-[1-[3-(Acetyl-methyl-amino)- phenylamino]-meth-(E/Z)-ylidene]- 3-ethyl-4-oxo-thiazolidin-(2-(E or Z))-ylidene}-2-cyano-N- cyanomethyl-acetamide | (DMSO-d6, stored via K₂CO₃, primary isomer): δ = 1.25 (t, 3H); 1.80 (3H); 3.15 (t, 1H); 3.15 (3H); 4.14 (d, 2H); 4.22 (q, 2H); 6.98 (d, 1H); 7.22-7.24 (m, 1H); 7.33-7.37 (m, 2H); 8.18 (d, 1H); 8.33 (t, 1H); 10.37 (d, 1H) ppm. | 424.48/ 425 | INTA17/ 1 |
| 168 | 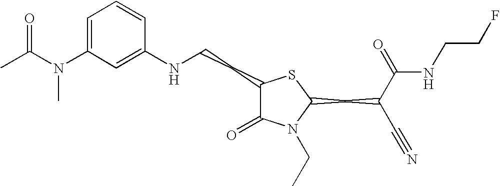 2-{5-[1-[3-(Acetyl-methyl-amino)- phenylamino]-meth-(E/Z)-ylidene]- 3-ethyl-4-oxo-thiazolidin-(2-(E or Z))-ylidene}-2-cyano-N-(2-fluoro- ethyl)-acetamide | (DMSO-d6, stored via K₂CO₃, primary isomer): δ = 1.24 (t, 3H); 1.80 (3H); 3.15 (s, 3H); 3.43-3.53 (2q, 2H); 4.22 (q, 2H); 4.40-4.55 (2t, 2H); 6.97 (d, 1H); 7.21-7.37 (m, 3H); 7.78 (t, 1H); 8.13 (1H); 10.23 (d, 1H) ppm. | 431.49/ 432 | INTA17/ 1 |
| 169 | 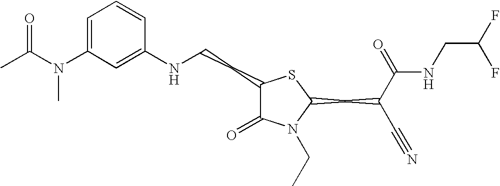 2-{5-[1-[3-(Acetyl-methyl-amino)- phenyl-amino]-meth-(E/Z)-ylidene]- 3-ethyl-4-oxo-thiazolidin-(2-(E or Z))-ylidene}-2-cyano-N-(2,2- difluoro-ethyl)-acetamide | (DMSO-d6, stored via K₂CO₃₋, primary isomer): δ = 1.25 (t, 3H); 1.81 (3H); 3.15 (s, 3H); 3.50-3.63 (m, 2H); 4.23 (q, 2); 5.90-6.20 (d, 1H); 6.99 (d, 1H); 7.23-7.39 (m, 3H); 7.95 (t, 1H); 8.17 (s, 1H); 10.32 (1H) ppm. | 449.48/ 450 | INTA17/ 1 |
| 170 | 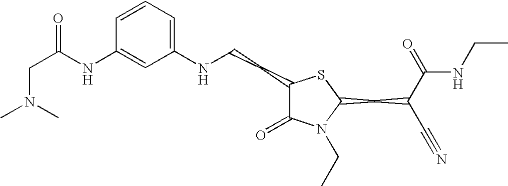 2-Cyano-2-{5-[1-[3-(2-dimethyl- amino-acetylamino)-phenylamino]- meth-(E/Z)-ylidene]-3-ethyl-4-oxo- thiazolidin-(2-(E or Z))-ylidene}-N- ethyl-acetamide | (DMSO-d6, stored via K₂CO₃, primary isomer): δ = 1.06 (t, 3H); 1.24 (t, 3H); 2.28 (s, 2H); 3.07 (s, 2H); 3.20 (p, 2H); 4.21 (q, 2H); 6.94 (d, 1H); 7.20-7.31 (m, 2H); 7.68 (t, 1H); 7.74 (s, 1H); 7.99 (d, 1H); 9.76 (s, 1H); 10.35 (d, 1H)ppm. | 442.54/ 443 | INTA18/ 1 |
| 171 | 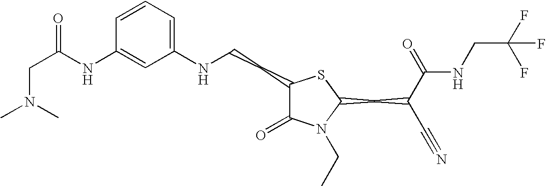 2-Cyano-2-{5-[1-[3-(2-dimethyl- amino-acetylamino)-phenylamino]- meth-(E/Z)-ylidene]-3-ethyl-4-oxo- thiazolidin-(2-(E or Z))-ylidene}-N- (2,2,2-trifluoro-ethyl)-acetamide | (DMSO-d6, stored via K₂CO₃, primary isomer): δ = 1.25 (t, 3H); 2.28 (s, 6H); 3.07 (s, 2H); 3.79-3.98 (m, 4H); 4.23 (q, 2H); 6.80 (t, 1H); 6.96 (m, 1H); 7.22-7.32 (m, 2H); 7.76 (s, 1H); 8.05 (d, 1H); 8.19 (t, 1H); 9.77 (s,1H); 10.47 (d, 1H) ppm. | 496.52/ 497 | INTA18/ 1 |
| 172 | 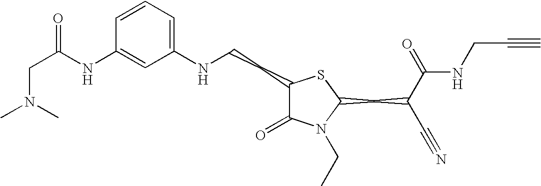 2-Cyano-2-{5-[1-[3-(2-dimethyl- amino-acetylamino)-phenylamino]- meth-(E/Z)-ylidene]-3-ethyl-4-oxo- thiazolidin-(2-(E or Z))-ylidene}-N- prop-2-ynyl-acetamide | (DMSO-d6, stored via K₂CO₃, primary isomer): δ = 1.24 (t, 3H); 2.28 (s, 6H); 3.05 (t, 1H); 3.07 (s, 2H); 3.91-3.93 (m, 2H); 4.22 (q, 2H); 6.96 (d, 1H); 7.22-7.33 (m, 2H); 7.76 (s, 1H); 8.;03 (d, 1H); 8.08 (t, 1H); 9.77 (s, 1H); 10.43(d, 1H) ppm. | 452.54/ 453 | INTA18/ 1 |
| 173 | 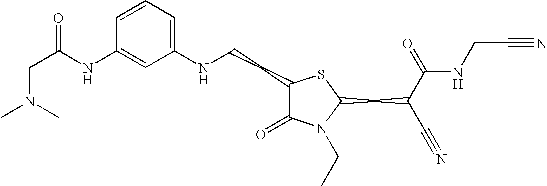 2-Cyano-N-cyanomethyl-2-{5-[1-[3- (2-dimethylamin-acetylamino)- phenylamino]-meth-(E/Z)-ylidene]- 3-ethyl-4-oxo-thiazolidin-(2-(E or Z))-ylidene}-acetamide | (DMSO-d6, stored via K₂CO₃, primary isomer): δ = 1.25 (t, 3H); 2.28 (s, 6H); 3.07 (s, 2H); 4.14-4.16 (m, 2H); 4.23 (q, 2H); 6.97 (d, 1H); 7.23-7.33 (m, 2H); 7.77 (s, 1H); 8.07 (d, 1H); 8.33 (t, 1H); 9.78 (1H); 10.51 (1H) ppm. | 453.53/ 454 | INTA18/ 1 |
| 174 | 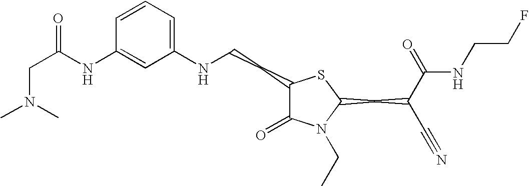 2-Cyano-2-{5-[1-[3-(2-dimethyl- amino-acetylamino)-phenylamino]- meth-(E/Z)-ylidene]-3-ethyl-4-oxo- thiazolidin-(2-(E or Z))-ylidene}-N- (2-fluoro-ethyl)-acetamide | (DMSO-d6, stored via K₂CO₃, primary isomer): δ = 1.24 (t, 3H); 2.27 (s, 6H); 3.06 (s, 2H); 3.43-3.53 (2q, 2H); 4.22 (q, 2H); 4.40-4.55 (2t, 2H); 6.93-6.95 (m, 1H); 7.20-7.31 (m, 2H); 7.74-7.78 (m, 2H); 8.01 (d, 1H); 9.75 (s, 1H); 10.38 (d, 1H) ppm. | 460.53/ 461 | INTA18/ 1 |
| 175 | 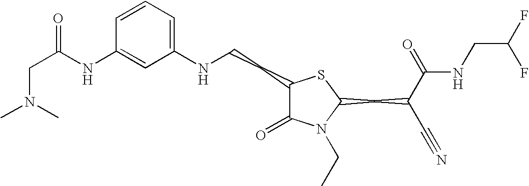 2-Cyano-N-(2,2-difluoro-ethyl)-2- {5-[1-[3-(2-dimethylamino- acetylamino)-phenylamino]-meth- (E/Z)-ylidene]-3-ethyl-4-oxo- thiazolidin-(2-(E or Z))-ylidene}- acetamide | (DMSO-d6, stored via K₂CO₃, primary isomer): δ = 1.24 (t, 3H); 2.27 (s, 6H); 3.06 (s, 2H); 3.52-3.62 (m, 2H); 4.22 (q, 2H); 5.89-6.19 (3t, 1H); 6.94 (d, 1H); 7.20-7.31 (m, 2H); 7.37 (1H); 7.91 (1H); 8.04 (1H); 9.75 (s,1H); 10.44 (s, 1H) ppm. | 478.52/ 479 | INTA18/ 1 |
| 176 | 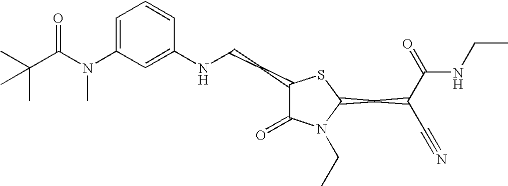 2-Cyano-2-[5-[1-{3-[(2,2-dimethyl- propionyl)-methyl-amino]-phenyl- amino}-meth-(E/Z)-ylidene]-3-ethyl- 4-oxo-thiazolidin-(2-(E or Z))- ylidene]-N-ethyl-acetamide | (DMSO-d6, stored via K₂CO₃, primary isomer): δ = 0.99 (s,9H); 1.07 (t, 3H); 1.24 (t, 3H); 3.10 (s, 3H); 3.17-3.23 (m, 2H); 4.22 (q, 2H); 6.97 (d, 1H); 7.27-7.38 (m, 2H); 7.70 (t, 1H); 8.11 (s, 1H); 10.26 (s, 1H) ppm. | 455.58/ 456 | INTA19/ 1 |
| 177 | 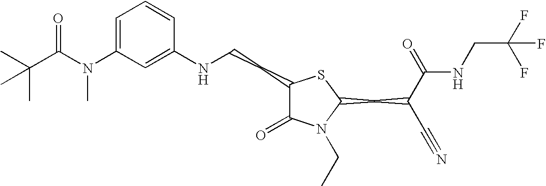 2-Cyano-2-[5-[1-{3-[(2,2-dimethyl- propionyl)-methyl-amino]-phenyl- amino}-meth-(E/Z)-ylidene]-3-ethyl- 4-oxo-thiazolidin-(2-(E or Z))- ylidene]-N-(2,2,2-trifluoro-ethyl)- acetamide | (DMSO-d6, stored via K₂CO₃, primary isomer): δ = 0.99 (s, 9H); 1.25 (t, 3H); 3.10 (s, 3H); 3.90-3.99 (m, 2H); 4.23 (q, 2H); 6.99 (d, 1H); 7.28-7.41 (m, 3H); 8.17 (d, 1H); 8.23 (t, 1H); 10.37 (d, 1H) ppm. | 509.55/ 510 | INTA19/ 1 |
| 178 | 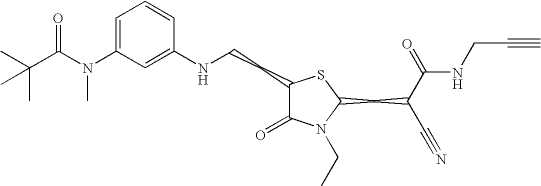 2-Cyano-2-[5-[1-{3-[(2,2-dimethyl- propionyl)-methyl-amino]-phenyl- amino}-meth-(E/Z)-ylidene]-3-ethyl- 4-oxo-thiazolidin-(2-(E or Z))- ylidene]-N-prop-2-ynyl-acetamide | (DMSO-d6, stored via K₂CO₃, primary isomer): δ = 1.00 (s, 9H); 1.24 (t, 3H); 3.06 (t, 1H); 3.10 (s, 3H); 3.91-3.93 (m, 2H); 4.22 (q, 2H); 6.98 (d, 1H); 7.28-7.41 (m, 3H); 8.11 (t, 1H); 8.14 (d, 1H); 10.32 (d, 1H) ppm. | 465.58/ 466 | INTA19/ 1 |
| 179 | 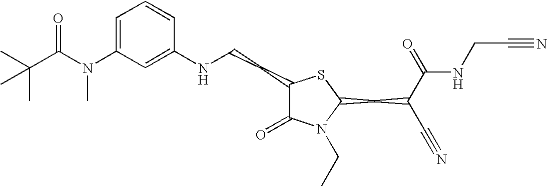 2-Cyano-2-[5-[1-{3-[(2,2-dimethyl- propionyl)-methyl-amino]-phenyl- amino}-meth-(E/Z)-ylidene]-3-ethyl- 4-oxo-thiazolidin-(2-(E or Z))- ylidene]-N-prop-2-ynyl-acetamide | (DMSO-d6, stored via K₂CO₃, primary isomer): δ = 1.00 (s, 9H); 1.26 (t, 3H); 3.12 (s, 3H); 4.16 (d, 2H); 4.24 (q, 2H); 7.00 (d, 1H); 7.30-7.43 (m, 3H); 8.19 (d, 1H); 8.37 (t, 1H); 10.41 (d, 1H) ppm. | 466.56/ 467 | INTA19/ 1 |
| 180 | 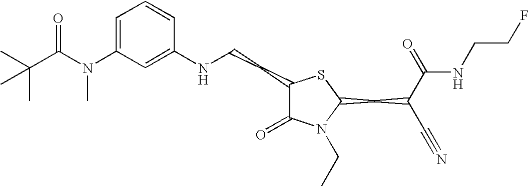 2-Cyano-2-[5-[1-{3-[(2,2-dimethyl- propionyl)-methyl-amino]-phenyl- amino}-meth-(E/Z)-ylidene]-3-ethyl- 4-oxo-thiazolidin-(2-(E or Z))- ylidene]-N-(2,2-trifluoro-ethyl)- acetamide | (DMSO-d6, stored via K₂CO₃, primary isomer): δ = 0.99 (s, 9H); 1.24 (t, 3H); 3.09 (s, 3H); 3.42-3.52 (2q, 2H); 4.21 (q, 2H); 4.41 (t, 1H); 4.53 (t, 1H); 6.95 (d, 1H); 7.24-7.36 (m, 3H); 7.78 (d, 1H); 8.12 (s,1H); 10.29 (s, 1H) ppm. | 473.57/ 474 | INTA19/ 1 |
| 181 | 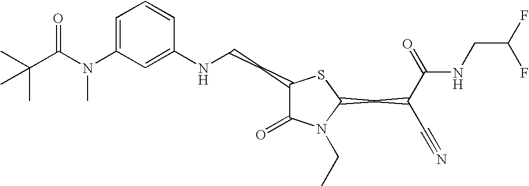 2-Cyano-N-(2,2-difluoro-ethyl)-2-[5- [1-{3-[(2,2-dimethyl-propionyl)- methyl-amino]-phenylamino}-meth- (E/Z)-ylidene]-3-ethyl-4-oxo- thiazolidin-(2-(E or Z))-ylidene]- acetamide | (DMSO-d6, stored via K₂CO₃, primary isomer): δ = 1.01 (s, 9H); 1.26 (t, 3H); 3.09 (s, 3H); 3.51-3.61 (m, 2H); 4.21 (q, 2H); 5.86-6.16 (3t, 1H); 6.94 (d, 1H); 7.23-7.37 (m, 3H); 7.94 (t, 1H); 8.12 (1H); 10.26 (1H)ppm. | 491.56/ 492 | INTA19/ 1 |
| 182 | 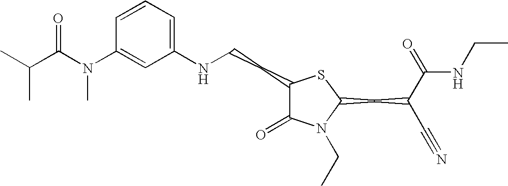 2-Cyano-N-ethyl-2-{3-ethyl-5-[1-[3- (isobutyryl-methyl-amino)-phenyl- amino]-meth-(E/Z)-ylidene]-4-oxo- thiazolidin-(2-(E or Z))-ylidene}- acetamide | (DMSO-d6, stored via K₂CO₃, primary isomer): δ = 0.93 (d, 6H); 1.07 (t, 3H); 1.24 (t, 3H); 2.47 (m, 1H); 3.13 (s, 3H); 3.17-3.23 (m, 2H); 4.20 (q,2H); 6.98 (d, 1H); 7.27-7.32 (m, 2H); 7.39 (t, 1H); 7.72 (t, 1H); 8.13 (d, 1H); 10.27 (d, 1H) ppm. | 441.56/ 442 | INTA20/ 1 |
| 183 | 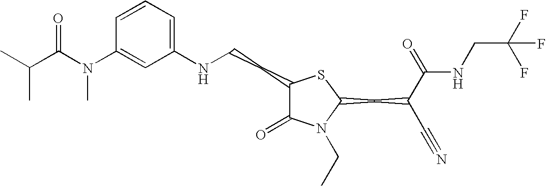 2-Cyano-2-{[3-ethyl-5-[1-[3- (isobutyryl-methyl-amino)-phenyl- amino]-meth-(E/Z)-ylidene]-4-oxo- thiazolidin-(2-(E or Z))-ylidene}-N- (2,2,2-trifluoro-ethyl)-acetamide | (DMSO-d6, stored via K₂CO₃, primary isomer): δ = 0.93 (d, 6H); 1.25 (t, 3H); 2.47 (m, H); 3.13 (s, 3H); 3.90-3.99 (m, 2H); 4.23 (q, 2H); 6.99 (d, 1H); 7.28-7.43 (m, 3H); 8.20-8.23 (m, 2H); 10.38 (1H) ppm. | 495.53/ 469 | INTA20/ 1 |
| 184 | 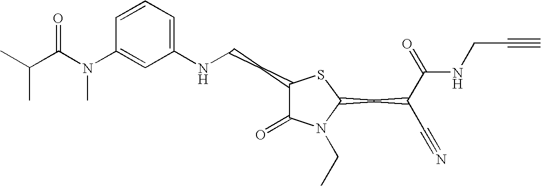 2-Cyano-2-{3-ethyl-5-[1-[3- (isobutyryl-methyl-amino)- phenylamino]-meth-(E/Z)-ylidene]- 4-oxo-thiazolidin-(2-(E or Z))- ylidene}-N-prop-2-ynyl-acetamide | (DMSO-d6, stored via K₂CO₃, primary isomer): δ = 0.93 (d, 6H); 1.24 (t, 3H); 2.47 (m, H); 3.06 (t, H); 3.14 (s, 3H); 3.91-3.93 (m, 2H); 4.22 (q, 2H); 6.98 (d, 1H); 7.28-7.33 (m, 2H); 7.39 (t, 1H); 8.11 (t, 1H); 8.16 (1H); 10.33 (1H) ppm. | 451.51/ 452 | INTA20/ 1 |
| 185 | 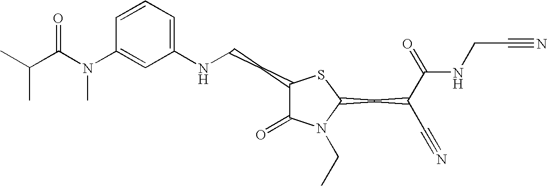 2-Cyano-N-cyanomethyl-2-{3-methyl- 5-[1-[3-(isobutyryl-methyl-amino)- phenylamino]-methyl-(E/Z)-ylidene]- 4-oxo-thiazolidin-(2-(E or Z))- ylidene]-acetamide | (DMSO-d6, stored via K₂CO₃, primary isomer): δ = 0.93 (d, 6H); 1.25 (t, 3H); 2.47 (m, H); 3.14 (s, 3H); 4.15 (d, 2H); 4.23 (q, 2H); 6.99 (d, 1H); 7.28-7.30 (m, 1H); 7.34-7.41 (m, 2H); 8.20 (1H); 8.36 (t, 1H); 10.40 (1H) ppm. | 452.54/ 453 | INTA20/ 1 |
| 186 | 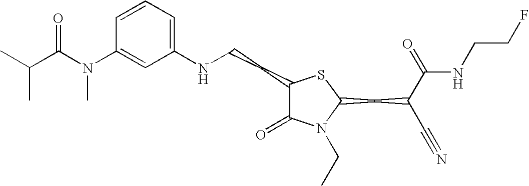 2-Cyano-2-{3-ethyl-5-[1-[3- (isobutyryl-methyl-amino)-phenyl- amino]-meth-(E/Z)-ylidene]-4-oxo- thiazolidin-(2-(E or Z))-ylidene}-N- 2-(fluoro-ethyl)-acetamide | (DMSO-d6, stored via K₂CO₃, primary isomer): δ = 0.93 (d, 6H); 1.24 (t, 3H); 2.47 (m, H); 3.13 (s, 3H); 3.44-3.54 (2q, 2H); 4.22 (q, 2H); (t, 1H); 4.54 (t, 1H); 6.97 (d, 1H); 7.25-7.31 (m, 2H); 7.27 (t, 1H); 7.79 (t, 1H); 8.13 (t,1H); 10.28 (1H) ppm. | 459.44/ 460 | INTA20/ 1 |
| 187 | 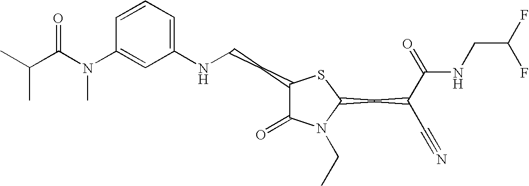 2-Cyano-N-(2,2-difluoro-ethyl)-2- {3-ethyl-5-[1-[3-(isobutyryl-methyl- amino)-phenylamino]-meth-(E/Z)- ylidene]-4-oxo-thiazolidin-(2-E or Z))-ylidene}-acetamide | (DMSO-d6, stored via K₂CO₃, primary isomer): δ = 0.93 (d, 6H); 1.24 (t, 3H); 2.47 (m, H); 3.13 (s, 3H); 3.53-3.62 (m, 2H); 4.23 (q, 2H); 5.90-6.20 (tt, 1H); 6.90 (d, 1H); 7.27-7.33 (m, 2H); 7.39 (t, 1H); 7.98 (t, 1H); 8.18 (1H); 10.37 (1H) ppm. | 477.54/ 478 | INTA20/ 1 |
| 188 | 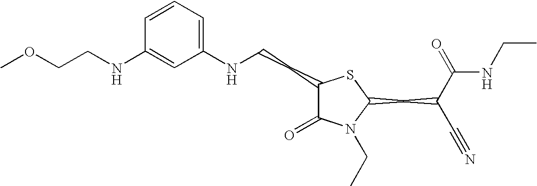 2-Cyano-N-ethyl-2-{3-ethyl-5-[1-[3- (2-methoxy-ethylamino)-phenyl- amino]-meth-(E/Z)-ylidene]-4-oxo- thiazolidin-(2-(E or Z))-ylidene}- acetamide | (DMSO-d6, stored via K₂CO₃, primary isomer): δ = 1.06 (t, 3H); 1.23 (t, 3H); 3.19 (q, 2H); 3.28 (s, 3H); 3.47 (t, 2H); 4.21 (q, 2H); 5.78 (t, 1H); 6.30-6.32 (dd, 1H); 6.41-6.44 (dd, 1H); 6.49 (t, 1H); 7.00 (t, 1H); 7.68 (t, 1H); 7.98 (d, 1H); 10.13 (d, 1H) ppm. | 415.52/ 416 | INTA21/ 1 |
| 189 | 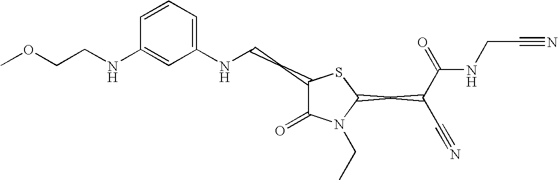 2-Cyano-N-cyanomethyl-2-[3-ethyl- 5-[1-[3-(2-methoxy-methylamino)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.24 (t, 3H); 3.19 (q, 2H); 3.28 (s, 3H); 3.47 (t, 2H); 4.14 (d, 2H); 4.22 (q, 2H); 5.78 (t, 1H); 6.31-6.33 (dd, 1H); 6.42-6.45 (dd, 1H); 6.49 (t, 1H); 7.01 (t, 1H); 8.05 (s, 1H); 8.29 (1H); 10.27 (s, 1H) ppm. | 426.50/ 427 | INTA21/ 1 |
| 190 | 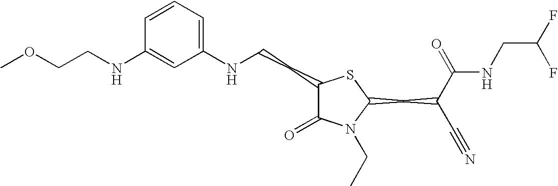 2-Cyano-N-(2,2-difluoro-ethyl)-2-[3- ethyl-5-[1-[3-(2-methoxy- ethylamino)-phenylamino]-meth- (E/Z)-ylidene]-4-oxo-thiazolidin-(2- (E or Z))-ylidene]-acetamide | (DMSO-d6, stored via K₂CO₃, primary isomer): δ = 1.24 (t, 3H); 3.19 (q, 2H); 3.28 (s, 3H); 3.47 (t, 2H); 3.53-3.63 (m, 2H); 4.22 (q, 2H); 5.78 (t, 1H); 5.90-6.20 (tt, 1H); 6.31-6.33 (dd, 1H); 6.42-6.44 (dd, 1H); 6.49 (t, 1H); 7.01 (t,1H); 7.92 (t, 1H); 8.02 (d, 1H); 10.22 (d, 1H) ppm. | 451.50/ 452 | INTA21/ 1 |
| 191 | 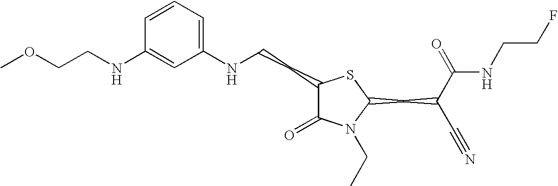 2-Cyano-2-[3-ethyl-5-[1-[3-(2- methoxy-ethylamino)- phenylamino]-meth-(E/Z)-ylidene]- 4-oxo-thiazolidin-(2-(E or Z))- ylidene]-N-(2-fluoro-ethyl)- acetamide | (DMSO-d6, stored via K₂CO₃, primary isomer): δ = 1.24 (t, 3H); 3.18 (q, 2H); 3.28 (s, 3H); 3.43-3.53 (m, 4H); 3.53-3.63 (m, 2H); 4.22 (q, 2H); 4.42 (t, 1H); 4.54 (t, 1H); 5.77 (t, 1H); 6.30-6.33 (dd, 1H); 6.41-6.44 (dd, 1H); 6.49 (1H); 7.00 (t, 1H); 7.76 (t, 1H); 8.00 (d, 1H); 10.16 (d, 1H) ppm. | 433.51/ 434 | INTA21/ 1 |
| 192 | 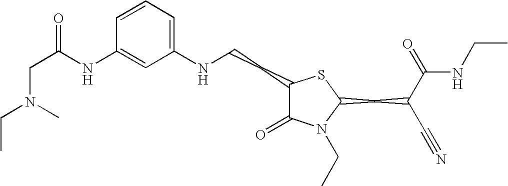 2-Cyano-N-ethyl-2-[3-ethyl-5-[1-{3- [2-(ethyl-methyl-amino)-acetyl- amino]-phenylamino}-meth-(E/Z)- ylidene]-4-oxo-thiazolidin-(2-(E or Z))-ylidene]-acetamide | (DMSO-d6, stored via K₂CO₃, primary isomer): δ = 1.02-1.08 (m, 6H); 1.23 (t, 3H); 2.28 (s, 3H); 2.51 (q, 2H); 3.11 (s, 2H); 3.16-3.23 (m, 2H); 4.21 (q, 2H); 6.93-6.96 (m, 1H); 7.21-7.31 (m, 2H); 7.68 (t, 1H); 7.73 (1H); 8.00 (d,1H); 9.71 (s, 1H); 10.35 (d, 1H) ppm. | 456.57/ 457 | INTA22/ 1 |
| 193 | 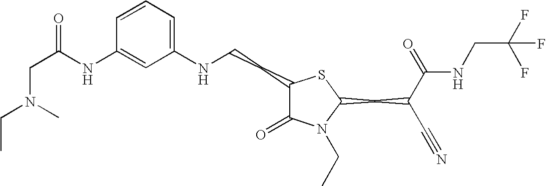 2-Cyano-2-[3-ethyl-5-[1-{3-[2- (ethyl-methyl-amino)-acetylamino]- phenylamino}-meth-(E/Z)-ylidene]- 4-oxo-thiazolidin-(2-(E or Z))- ylidene]-N-(2,2,2-trifluoro-ethyl)- acetamide | (DMSO-d6, stored via K₂CO₃primary isomer): δ = 1.04 (t, 3H); 1.25 (t, 3H); 2.27 (s, 3H); 2.51 (m, 2H); 3.11 (s, 2H); 3.90-3.98 (m, 2H); 4.23 (q, 2H); 6.96 (d, 1H); 7.22-7.32 (m, 2H); 7.74 (s, 1H); 8.07 (1H); 8.19 (1H); 9.71 (s, 1H); 10.46 (1H) ppm. | 510.54/ 511 | INTA22/ 1 |
| 194 | 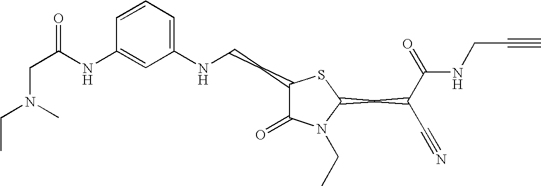 2-Cyano-2-[3-ethyl-5-[1-{3-[2- (ethyl-methyl-amino)-acetylamino]- phenylamino}-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 1.04 (t, 3H); 1.24 (t, 3H); 2.28 (s, 3H); 2.51 (q, 2H); 3.12 (s, 2H); 3.91-3.92 (dd, 2H); 4.22 (q, 2H); 6.95-6.97 (m, 1H); 7.22-7.31 (m, 2H); 7.73 (s, 1H); 8.03 (d, 2H); 8.07(t, 1H); 9.72 (s, 1H); 10.41 (d, 1) ppm. | 466.57/ 467 | INTA22/ 1 |
| 195 | 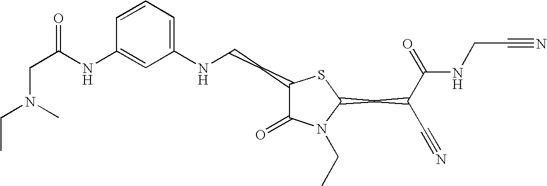 2-Cyano-N-cyanomethyl-2-[3-ethyl- 5-[1{3-[2-(ethyl-methyl-amino)- acetylamino]-phenylamino}-meth- (E/Z)-ylidene]-4-oxo-thiazolidin-(2- (E or Z))-ylidene]-acetamide | (DMSO-d6, stored via K₂CO₃, primary isomer): δ = 1.04 (t, 3H); 1.25 (t, 3H); 2.28 (s, 3H); 2.51 (m, 2H); 3.12 (s, 2H); 4.15 (d, 2H); 4.23 (q, 2H); 6.96-6.98 (m, 1H); 7.22-7.32 (m, 2H); 7.75 (1H); 8.07 (d, 2H); 8.33 (t, 1H); 9.72 (1H); 10.50 (d, 1H) ppm. | 467.55/ 468 | INTA22/ 1 |
| 196 | 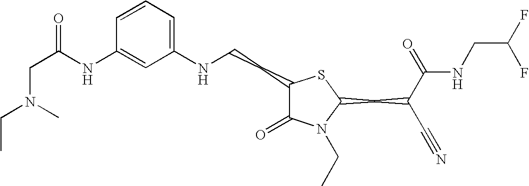 2-Cyan-N-(2,2-difluoro-ethyl)-2-[3- ethyl-5-[1-{3-[2-(ethyl-methyl- amino)-acetylamino]-phenylamino}- meth-(E/Z)-ylidene]-4-oxo- thiazolidin-(2-E or Z))-ylidene]- acetamide | (DMSO-d6, stored via K₂CO₃, primary isomer): δ = 1.04 (t, 3H); 1.25 (t, 3H); 2.28 (s, 2H); 2.51 (m, 2H); 3.11 (s, 2H); 3.54-3.62 (m, 2H); 4.23 (q, 2H); 5.90-6.20 (tt, 1H); 6.95-6.97 (m, 1H); 7.22-7.32 (m, 2H); 7.74 (s,1H); 7.95 (t, 1H); 8.05 (d, 1H); 9.71 (s, 1H); 10.44 (d, 1H) ppm. | 492.55/ 493 | INTA22/ 1 |
| 197 | 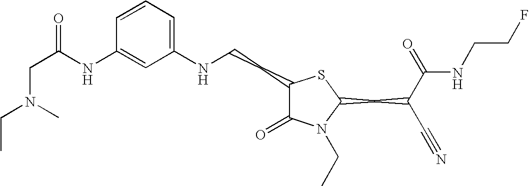 2-Cyan-2-[3-ethyl-5-[1-{3-[2- (ethyl-methyl-amino)-acetylamino]- phenylamino}-methyl-(E/Z)-ylidene]- 4-oxo-thiazolidin-(2-(E or Z))- ylidene]-N-(2-fluoro-ethyl)- acetamide | (DMSO-d6, stored via K₂CO₃, primary isomer): δ = 1.04 (t, 3H); 1.254 (t, 3H); 2.28 (s, 3H); 2.51 (m, 2H); 3.12 (s, 2H); 3.43-3.53 (2q, 2H); 4.22 (q, 2H); 4.42 (t, 1H); 4.54 (t, 1H); 6.94-6.96 (m, 1H); 7.21-7.31 (m, 2H); 7.73 (s, 1H); 7.79 (t, 1H); 8.02 (d, 1H); 9.71 (s, 1H); 10.40 (d, 1H) ppm. | 474.56/ 475 | INTA22/ 1 |

### Example 198

### Acetic acid (3-{[2-[1-cyano-1-prop-2-ynylcarbamoyl-meth-(E or Z)-ylidene]-3-ethyl-4-oxo-thiazolidin-(5-(E/Z))-ylidenemethyl]-amino}-phenylcarbamoyl)-methyl ester

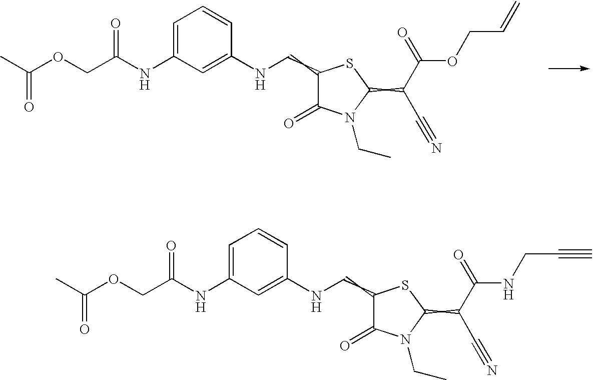

Dissolve 2.5 g of the compound described under Intermediate INTE44 in 160 ml tetrahydrofurane, add 1.66 g N,N-dimethyl barbituric acid and 614 mg Pd(PPh₃)₄ to it and stir for two hours at room temperature. Following this 3.68 ml triethylamine, add 1.09 ml propargylamine and 5.12 g TBTU and stir for a further 15 hours at room temperature. Add 250 ml acetic acid ethylester and wash once again with 100 ml water. Dry the organic phase over sodium sulfate. After purification through re-crystallization from dichlormethane and additional re-crystallization from ethanol, 1.68 g of the compound in the title is obtained.

¹H-NMR (DMSO-d6, stored over K₂CO₃, primary isomer): δ=1.25 (t, 3H); 2.14 (s, 3H); 3.07 (t, 1H); 3.88-4.00 (m, 2H); 4.24 (q, 2H); 4.66 (s, 2H); 7.02 (d, 1H); 7.20 (d, 1H); 7.29 (t, 1H); 7.67 (s, 1H); 8.02 (d, 1H); 8.11 (t, 1H); 10.16 (s, 1H); 10.46 (d, 1H) ppm.

### Example 199

### 2-Cyano-2-[3-ethyl-5-[1-[3-(2-hydroxy-acetylamino)-phenylamino]-meth-(E/Z)-ylidene]-4-oxo-thiazolidin-(2-(E or Z))-ylidene]-N-prop-2-ynyl-acetamide

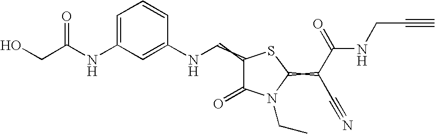

Dissolve 2.6 g of the compound described under Example 198 in 80 ml dimethylformamide and add 40 ml methanol and 40 ml water to it. Add 1.15 g of potassium carbonate and stir for two hours at room temperature. Add 1000 ml acetic acid ethylester, the organic phase is separated and washed thrice with 75 ml of a semi-saturated sodium chloride solution each time. Dry the organic phase over sodium sulfate. 2.19 g of the compound in the title is obtained.

(DMSO-d6, stored via K₂CO₃, primary isomer): δ=1.21 (t, 3H); 3.02 (b, 1H); 3.83-3.93 (m, 2H); 3.96 (d, 2H); 4.19 (q, 2H); 5.67 (t, 1H); 6.94 (d, 1H); 7.22 (t, 1H); 7.35 (d, 1H); 7.77 (s, 1H); 7.94-8.12 (m, 2H); 9.70 (s, 1H); 10.40 (d, b, 1H) ppm.

### Example 200

### Methanesulfonic acid (3-{[2-[1-cyano-1-prop-2-ynylcarbamoyl-meth-(E or Z)-ylidene]-3-ethyl-4-oxo-thiazolidin-(5-(E/Z))-ylidenemethyl]-amino}-phenylcarbamoyl)-methyl ester

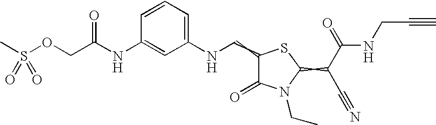

Dissolve 2.18 g of the compound described under Example 199 in 18 ml dimethylformamide and add 320 ml tetrahydrofurane to it. At 0° C., add 1.78 ml triethylamine and 0.60 ml methanesulfonic acid chloride and stir for one hour at room temperature. Add 500 ml acetic acid ethylester and 200 ml water, separate the organic phase and wash thrice with 75 ml of a semi-saturated sodium chloride solution each time. Dry the organic phase over sodium sulfate. After purification through a stirring out of the solids with dichlormethane, 2.02 g of the compound in the title is obtained.

(DMSO-d6, stored via K₂CO₃, primary isomer): δ=1.24 (t, 3H); 3.06 (b, 1H); 3.31 (s, 3H); 3.86-3.99 (m, 2H); 4.22 (q, 2H); 4.85 (s, 2H); 7.04 (d, 1H); 7.22 (d, 1H); 7.30 (t, 1H); 7.68 (s, 1H); 8.03 (d, 1H); 8.10 (t, 1H); 10.24 (s, 1H); 10.47 (d, b, 1H) ppm.

### Example 201

### 2-cyano-N-cyanomethyl-2-[5-[1-{3-[2-(4,4-difluoro-piperidin-1-yl)-acetylamino]-4-fluoro-phenylamino}-meth-(E/Z)-ylidene]-3-ethyl-4-oxo-thiazolidin-(2-(E or Z))-ylidene]-acetamide

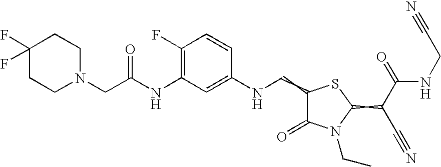

Suspend 60 mg of the compound described under INTT10 in 3 ml 1-propanol, add 138 mg of the compound described under INT62 and 0.16 ml triethylorthoformiate to it. Stir for 4 hours at 140° C. in a bomb tube. Allow the reaction mixture to gradually cool at room temperature and stir for 15 hours at room temperature. Filter off the excluded solids and wash successively with ethanol and diethylether. After purification through filtration through silica gel and subsequent re-crystallization from ethanol, 106 mg of the compound in the title are obtained.

(DMSO-d6, stored via K₂CO₃, primary isomer): δ=1.20 (t, 3H); 1.83-2.10 (m, 4H); 2.66 (m, 4H); 3.26 (s, 2H); 4.11 (d, 2H); 4.19 (q, 2H); 6.95-7.12 (m, 1H); 7.22 (t, 1H); 7.93 (s, b, 1H); 8.02 (s, 1H); 8.27 (s, b, 1H); 9.62 (s, 1H); 10.50 (s, b, 1H) ppm.

### Example 202

### 2-[5-[1-(3-Amino-phenylamino)-meth-(E/Z)-ylidene]-3-ethyl-4-oxo-thiazolidin-(2-(E or Z))-ylidene]-2-cyano-N-(2,2,2-trifluoro-ethyl)-acetamide

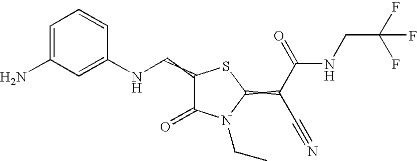

Suspend 1.6 g of the compound described under Example 204 in 40 ml dichlormethane. Add 24 ml trifluoro-acetic acid to it and stir for one hour at room temperature. Press reaction mixture, add dichlormethane and hexane and press anew. After drying well in vacuum, 1.7 g of the compound in the title is obtained in the form of trifluoro acetic acid salt. This raw product is used without further purification for the following reactions.

### Example 203

### 2-[5-[1-[3-(2-chloro-acetylamino)-phenylamino]-meth-(E/Z)-ylidene]-3-ethyl-4-oxo-thiazolidin-(2-(E or Z))-ylidene]-2-cyano-N-(2,2,2-trifluoro-ethyl)-acetamide

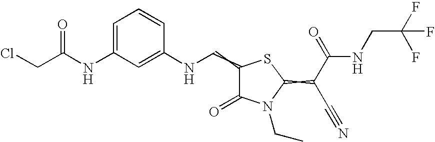

Dissolve 3.1 mmol of the trifluoro-acetic acid salts of the compound described under Example 202 in 45 ml tetrahydrofurane. At 0° C., add 0.64 ml pyridine and 0.60 mg chloro-acetic acid anhydride and stir for 30 minutes at room temperature. Add 200 ml acetic acid ethylester and 100 ml water, separate the organic phase and dry over sodium sulfate. After purification through the re-crystallizing of ethanol, 1.12 g of the compound in the title is obtained.

(DMSO-d6, stored via K₂CO₃, primary isomer): δ=1.27 (t, 3H); 3.98 (m, 2H); 4.19-4.31 (m, 4H); 7.04 (d, 1H); 7.22 (d, 1H); 7.31 (t, 1H); 7.70 (s, 1H); 8.06 (b, 1H); 8.21 (b, 1H); 10.40 (s, 1H); 10.54 (s, b, 1H) ppm.

### Example 204

### N-Allyl-2-[5-[1-{3-[2-(4-benzyl-piperazin-1-yl)-2-oxo-ethoxy]-phenylamino}-meth-(E/Z)-ylidene]-3-ethyl-4-oxo-thiazolidin-(2-(E or Z))-ylidene]-2-cyano-acetamide

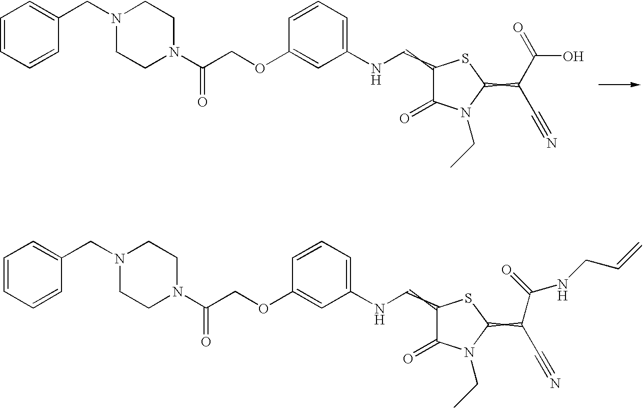

Dissolve 95 mg of the compound described under INTA23 in 3 ml DMF and add HATU (194 mg) as well as allylamine (34 μl). Stir the reaction residue at room temperature under argon over night, diluted with water (approx. 20 ml), made alkaline through the addition of a sodium carbonate solution and extracted with acetic ester (3×10 ml). Dry the unified organic phases over sodium sulfate and distill the solvent on a rotary evaporator Purify the raw product chromatographically on the Flashmaster. The compound in the title (45 mg) is obtained in a 45% yield.

¹H-NMR (CDCl₃, primary isomer): δ=1.39 (m, 3H); 2.49 (m, 4H); 3.61 (m, 4H); 3.69 (m, 2H); 3.97 (m, 2H); 4.38 (m, 2H); 4.80 (s, 2H); 5.21 (m, 2H); 5.88 (m, 1H); 6.38 (t, 1H); 6.58 (m, 3H); 7.12 (t, 1H); 7.50 (m, 2H); 7.68 (m, 1H); 8.00 (d, 1H); 8.65 (d, 1H); 10.40 (d, 1H) ppm.

The following compounds are manufactured according to the process described above.

| | | | Molecular | |
|---|---|---|---|---|
| | | | Weight/ | Educt/ |
| Example | | | MS (ESI) | Additional |
| no. | Structure and name | ¹H-NMR | [M + 1]⁺ | synthesis |
| 205 | 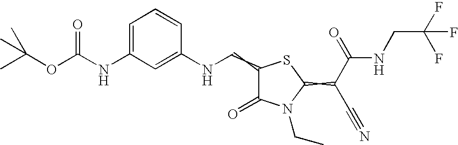 (3-{[2-[1-Cyano-1-(2,2,2-trifluoro- ethylcarbamoyl)-meth-(E or Z)- ylidene]-3-ethyl-4-oxo-thiazolidin- (5-(E/Z))-ylidenemethyl]-amino}- phenyl)-carbamic acid tert-butyl ester | (DMSO-d6, stored via K₂CO₃, primary isomer): δ = 1.25 (t, 3H); 1.49 (s, 9H); 3.97 (m, 2H); 4.25 (q, 2H); 6.90 (d, 1H); 7.07 (d, 1H); 7.21 (t, 1H); 7.57 (s, 1H); 8.03 (b, 1H); 8.22 (b, 1H); 9.45 (s, 1H); 10.50 (s, b, 1H) ppm | | INTA9/1 |
| 206 | 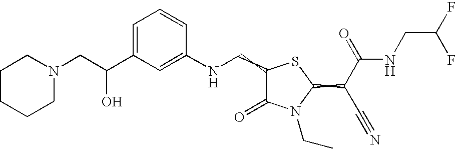 2-Cyano-N-(2,2-difluoro-ethyl)-2-[3- ethyl-5-[1-[3-(1-hydroxy-2-piperidin- 1-yl-ethyl)-phenylamino]-meth- (E/Z)-ylidene]-4-oxo-thiazolidin-(2- (E or Z))-ylidene]-acetamide | (DMSO-d6, stored via K₂CO₃, primary isomer): δ = 1.21 (t, 3H); 1.27-1.56 (m, 6H); 2.27-2.42 (m, 6H); 3.45-3.63 (m, 2H); 4.20 (q, 2H); 4.65 (s, b, 1H); 4.95 (s, b, 1H); 6.02 (tt, 1H), 6.99 (d, 1H); 7.10 (d, 1H); 7.17-7.33 (m, 2H); 7.90 (s, b, 1H); 8.09 (s, 1H); 10.37 (s, b, 1H) ppm | | INTT11/INT54/5 |
| 207 | 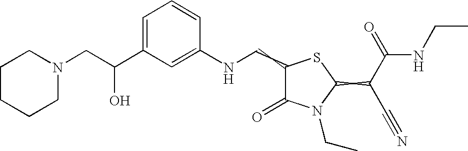 2-Cyano-N-ethyl-2-[3-ethyl-5-[1-[3- (1-hydroxy-2-piperidin-1-yl-ethyl)- phenylamino]-meth-(E/Z)-ylidene]- 4-oxo-thiazolidin-(2-(E or Z))- ylidene]-acetamide | (DMSO-d6, stored via K₂CO₃, primary isomer): δ = 1.03 (t, 3H); 1.20 (t, 3H); 1.27-1.54 (m, 6H); 2.23-2.44 (m, 6H); 3.16 (m, 2H); 4.18 (q, 2H); 4.62-4.66 (m, 1H); 4.94 (s, b, 1H); 6.98 (d, 1H); 7.08 (d, 1H); 7.17-7.33 (m, 2H); 7.62 (s, b, 1H); 8.05 (s, 1H); 10.27 (s, b, 1H) ppm | | INTT7/INT54/5 |
| 208 | 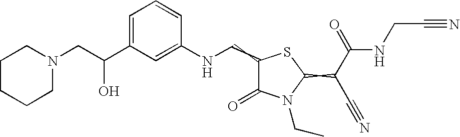 2-Cyano-N-cyanomethyl-2-[3-ethyl- 5-[1-[3-(1-hydroxy-2-piperidin-1-yl- ethyl)-phenylamino]-meth-(E/Z)- ylidene]-4-oxo-thiazolidin-(2-(E or Z))-ylidene]-acetamide | (DMSO-d6, stored via K₂CO₃, primary isomer): δ = 1.21 (t, 3H); 1.28-1.56 (m, 6H); 2.24-2.43 (m, 6H); 4.12 (d, 2H); 4.19 (q, 2H); 4.65 (s, b, 1H); 4.95 (s, b, 1H); 7.00 (d, 1H); 7.13 (d, 1H); 7.17-7.33 (m, 2H); 8.11 (s, 1H); 8.28 (s, b, 1H); 10.41 (s, b, 1H) ppm | | INTT10/INT54/5 |
| 209 | 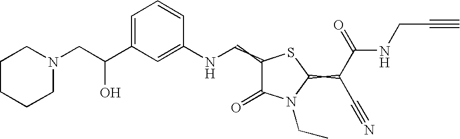 2-Cyano-2-[3-ethyl-5-[1-[3-(1- hydroxy-2-piperidin-1-yl-ethyl)- phenylamino]-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 1.20 (t, 3H); 1.27-1.54 (m, 6H); 2.24-2.44 (m, 6H); 3.02 (m, 1H); 3.88 (m, 2H); 4.19 (q, 2H); 4.65 (s, b, 1H); 4.94 (s, b, 1H); 6.99 (d, 1H); 7.10 (d, 1H); 7.16-7.32 (m, 2H); 7.95-8.14 (m, 2H); 10.33 (s, b, 1H); ppm | | INTT9/INT54/5 |
| 210 | 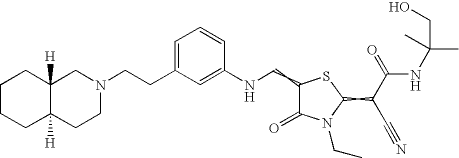 2-Cyano-2-[3-ethyl-5-[1-{3- [(4aR,8aS)-2-(octahydro- isoquinolin-2-yl)-ethyl]- phenylamino}-meth-(E/Z)-ylidene]- 4-oxo-thiazolidin-(2-(E or Z))- ylidene]-N-(2-hydroxy-1,1-dimethyl- ethyl)-acetamide | (DMSO-d6, stored via K₂CO₃, primary isomer): δ = 0.70-1.30 (m, 16H); 1.40-1.71 (m, 6H); 1.88 (t, 1H); 2.35-2.50 (m, 2H); 2.60-2.71 (m, 1H); 2.75 (d, 1H); 2.88 (d, 1H); 3.33 (d, 2H); 4.16 (q, 2H); 5.14 (t, 1H); 6.60 (s, b, 1H); 6.86 (d, 1H); 6.97-7.23 (m, 3H); 8.09 (s, 1H); 10.20 (s, b, 1H) ppm | | INTA24/1 |
| 211 | 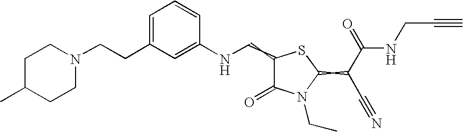 2-Cyano-2-[3-ethyl-5-[1-{3-[2-(4- methyl-piperidin-1-yl)-ethyl]- phenylamino}-meth-(E/Z)-ylidene]- 4-oxo-thiazolidin-(2-(E or Z))- ylidene]-N-prop-2-ynyl-acetamide | (DMSO-d6, stored via K₂CO₃, primary isomer): δ = 0.84 (d, 3H); 0.99-1.36 (m, 6H); 1.53 (d, 2H); 1.88 (t, 2H); 2.35-2.50 (m, 2H); 2.60-2.71 (m, 2H); 2.78-2.90 (m, 2H); 3.00 (b, 1H); 3.82-3.92 (m, 2H); 4.19 (q, 2H); 6.82 (d, 1H); 6.95 (d, 1H); 7.02 (s, 1H); 7.14 (t, 1H); 7.74 (s, b, 1H); 8.13 (s, 1H); 10.24 (s, b, 1H) ppm | | 69/14 |
| 212 | 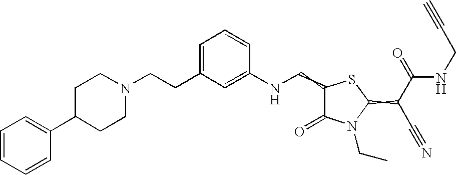 2-Cyano-2-[3-ethyl-4-oxo-5-[1-{3- [2-(4-phenyl-piperidin-1-yl)-ethyl]- phenylamino}-meth-(E/Z)-ylidene]- thiazolidin-(2-(E or Z))-ylidene]-N- prop-2-ynyl-acetamide | (DMSO-d6, stored via K₂CO₃, primary isomer): δ = 1.21 (t, 3H); 1.53-1.76 (m, 4H); 2.02 (t, 2H); 2.38-2.58 (m, 3H); 2.71 (t, 2H); 2.95-3.07 (m, 3H); 3.83-3.93 (m, 2H); 4.20 (q, 2H); 6.91 (d, 1H); 7.03-7.30 (m, 8H); 8.05 (s, b, 1H); 8.11 (s, 1H); 10.29 (s, b, 1H) ppm | | 69/14 |
| 213 | 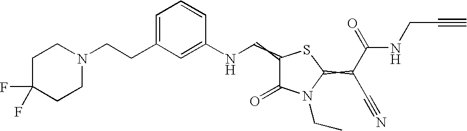 2-cyano-2-[5-[1-{3-[2-(4,4-difluoro- piperidin-1-yl)-ethyl]-phenylamino}- meth-(E/Z)-ylidene]-3-ethyl-4-oxo- thiazolidin-(2-(E or Z))-ylidene]-N- prop-2-ynyl-acetamide | (DMSO-d6, the chief isomer, stored over K₂CO₃): δ = 1.21 (t, 3H); 1.80-2.00 (m, 4H); 2.40-2.61 (m, 6H); 2.70 (t, 2H); 3.02 (b, 1H); 3.83-3.92 (m, 2H); 4.20 (q, 2H); 6.90 (d, 1H); 7.00-7.28 (m, 3H); 7.91-8.17 (m, 2H); 10.30 (s, b, 1H) ppm. | | 69/14 |
| 214 | 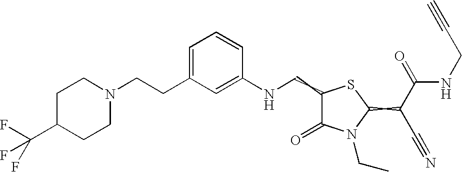 2-cyano-2-[3-ethyl-4-oxo-5-[1-{3-[2- (4-trifluoromethyl-piperidin-1-yl)- ethyl]-phenylamino}-meth-(E/Z)- ylidene]-thiazolidin-(2-(E orZ))- ylidene]-N-prop-2-ynyl-acetamide | (DMSO-d6, the chief isomer, stored over K₂CO₃): δ = 1.20 (t, 3H); 1.30-1.49 (m, 2H); 1.73 (d, 2H); 1.95 (t, 2H); 2.10-2.32 (m, 1H); 2.45-2.57 (m, 2H); 2.62-2.75 (m, 2H); 2.91-3.05 (m, 3H); 3.83-3.94 (m, 2H); 4.19 (q, 2H); 6.87 (d, 1H), 7.01 (d, 1H); 7.09 (s, 1H); 7.18 (t, 1H); 7.87 (s, b, 1H); 8.11 (s, 1H); 10.26 (s, b, 1H) ppm. | | 69/14 |
| 215 | 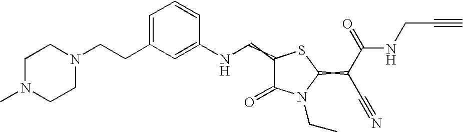 2-cyano-2-[3-ethyl-5-[1-{3-[2-(4- methyl-piperazin-1-yl)-ethyl]- phenylamino}-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 1.21 (t, 3H); 2.40-3.10 (m, b 15H); 3.02 (b, 1H); 3.83-3.96 (m, 2H); 4.20 (q, 2H); 6.92 (d, 1H); 7.09 (d, 1H); 7.15-7.29 (m, 2H); 8.00-8.15 (m, 2H); 10.27 (d, 1H) ppm. | | 69/14 |
| 216 | 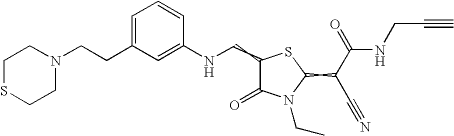 2-cyano-2-[3-ethyl-4-oxo-5-[1-[3-(2- thiomorpholin-4-yl-ethyl)- phenylamino]-meth-(E/Z)-ylidene]- thiazolidin-(2-(E or Z))-ylidene]-N- prop-2-ynyl-acetamide | (DMSO-d6, the chief isomer, stored over K₂CO₃): δ = 1.22 (t, 3H); 2.50-2.66 (m, 6H); 2.66-2.80 (m, 6H); 3.03 (m, 1H); 3.87-3.98 (m, 2H); 4.22 (q, 2H); 6.90 (d, 1H); 7.05 (d, 1H); 7.13 (s, 1H); 7.21 (t, 1H); 7.96 (s, b, 1H); 8.11 (s, 1H); 10.30 (s, b, 1H) ppm. | | 69/14 |
| 217 | 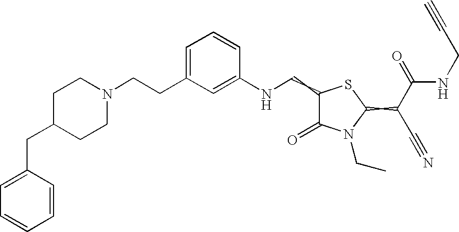 2-[5-[1-{3-[2-(4-benzyl-piperidin-1- yl)-ethyl]-phenylamino}-meth-(E/Z)- ylidene]-3-ethyl-4-oxo-thiazolidin- (2-(E or Z))-ylidene]-2-cyano-N- prop-2-ynyl-acetamide | (DMSO-d6, the chief isomer, stored over K₂CO₃): δ = 1.20 (t, 3H); 1.32-1.56 (m, 3H); 1.83 (t, 2H); 2.35-2.50 (m, 4H); 2.65 (t, 2H); 2.80-2.90 (m, 2H); 3.00 (m, 1H); 3.83-3.93 (m, 2H); 4.20 (q, 2H); 6.87 (d, 1H); 6.98-7.30 (m, 8H); 7.99 (s, b, 1H); 8.09 (s, 1H); 10.24 (s, b, 1H) ppm. | | 69/14 |
| 218 | 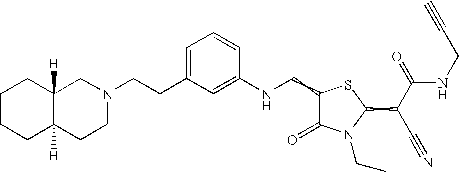 2-cyano-2-[3-ethyl-5-({3- [(4aR,8aS)-2-(octahydro- isoquinolin-2-yl)-ethyl]- phenylamino}-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 0.70-1.00 (m, 3H); 1.00-1.30 (m, 7H); 1.40-1.70 (m, 6H); 1.90 (t, 1H); 2.37-2.50 (m, 2H); 2.61-2.95 (m, 4H); 3.00 (b, 1H); 3.85-3.93 (m, 2H); 4.20 (q, 2H); 6.89 (d, 1H); 7.06 (d, 1H); 7.15 (s, b, 1H); 7.20 (t, 1H); 8.00 (s, b, 1H); 8.09 (s, 1H); 10.25 (s, b, 1H) ppm. | | 69/14 or INTA24/1 |
| 219 | 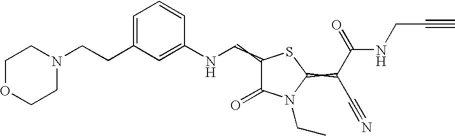 2-cyano-2-[3-ethyl-5-[1-[3-(2- morpholin-4-yl-ethyl)- phenylamino]-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 1.25 (t, 3H); 2.36-2.60 (m, 6H); 2.73 (t, 2H); 3.05 (b, 1H); 3.52-3.63 (m, 4H); 3.89-3.98 (m, 2H); 4.22 (q, 2H); 6.92 (d, 1H); 7.09 (d, 1H); 7.19 (s, b, 1H); 7.22 (t, 1H); 8.01 (s, b, 1H); 8.13 (s, 1H); 10.30 (s, b, 1H) ppm. | | 69/14 |
| 220 | 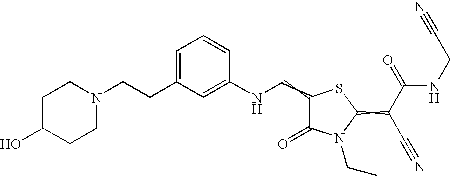 2-cyano-N-cyanomethyl-2-[3-ethyl- 5-[1-{3-[2-(4-hydroxy-piperidin-1- yl)-ethyl]-phenylamino}-meth-(E/Z)- ylidene]-4-oxo-thiazolidin-(2-(E or Z))-ylidene]-acetamide | (DMSO-d6, the chief isomer, stored over K₂CO₃): δ = 1.22 (t, 3H); 1.31-1.53 (m, b, 2H); 1.64-1.82 (m, b, 2H); 1.98-2.28 (m, b, 2H); 2.38-2.66 (m, b, 2H); 2.66-2.96 (m, b, 4H); 3.50 (b, 1H); 4.17 (d, 2H); 4.25 (q, 2H); 6.61 (b, 1H); 6.95 (d, 1H); 7.12 (d, 1H); 7.21 (s, b, 1H); 7.26 (t, 1H); 8.18 (s, b, 1H); 8.35 (t, 1H); 10.40 (s, b, 1H) ppm. | | 13/14 |
| 221 | 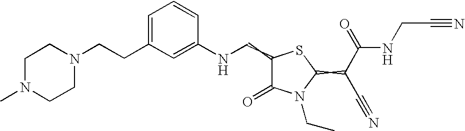 2-cyano-N-cyanomethyl-2-[3-ethyl- 5-[1-{3-[2-(4-methyl-piperazin-1-yl)- ethyl]-phenylamino}-meth-(E/Z)- ylidene]-4-oxo-thiazolidin-(2-(E or Z))-ylidene]-acetamide | (DMSO-d6, the chief isomer, stored over K₂CO₃): δ = 1.26 (t, 3H); 2.16 (s, 3H); 2.21-2.60 (m, 10H); 2.71 (t, 2H); 4.18 (d, 2H); 4.24 (q, 2H); 6.94 (d, 1H); 7.11 (d, 1H), 7.21 (s, b, 1H); 7.24 (t, 1H); 8.16 (s, b, 1H); 8.35 (t, 1H); 10.39 (s, b, 1H) ppm. | | 13/14 |
| 222 | 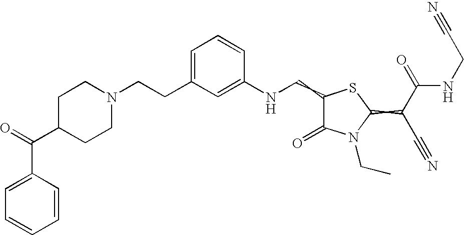 2-[5-[1-{3-[2-(4-benzoyl-piperidin-1- yl)-ethyl]-phenylamino}-meth-(E/Z)- ylidene]-3-ethyl-4-oxo-thiazolidin- (2-(E or Z))-ylidene]-2-cyano-N- cyanomethyl-acetamide | (DMSO-d6, the chief isomer, stored over K₂CO₃): δ = 1.21 (t, 3H); 1.48-1.88 (m, 4H); 2.14 (t, 2H); 2.45-2.60 (m, 2H); 2.65-2.80 (m, 2H); 2.90-3.07 (m, 2H); 3.30-3.48 (m, 1H); 4.11 (d, 2H); 4.22 (q, 2H); 6.74-7.25 (m, 4H); 7.47-7.70 (m, 3H); 7.90-8.06 (m, 3H); 8.23 (s, b, 1H); 10.40 (s, b, 1H) ppm. | | 13/14 |
| 223 | 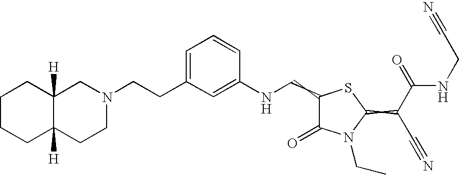 2-cyano-N-cyanomethyl-2-[3-ethyl- 5-[1-{3-[(4aS,8aS)-2-(octahydro- isoquinolin-2-yl)-ethyl]- phenylamino}-meth-(E/Z)-ylidene]- 4-oxo-thiazolidin-(2-(E or Z))- ylidene]-acetamide | (DMSO-d6, the chief isomer, stored over K₂CO₃): δ = 1.10-1.80 (m, 17H); 2.00-2.22 (m, 2H); 2.40-2.60 (m, 2H); 2.70 (t, 2H); 4.16 (d, 2H); 4.24 (q, 2H); 6.93 (d, 1H); 7.10 (d, 1H); 7.19 (s, b, 1H); 7.22 (t, 1H); 8.16 (s, 1H); 8.30 (s, b, 1H); 10.40 (s, b, 1H) ppm. | | 13/14 |
| 224 | 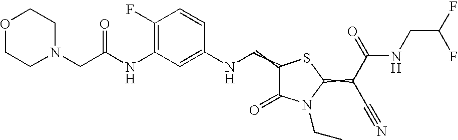 2-cyano-N-(2,2-difluoro-ethyl)-2-[3- ethyl-5-[1-[4-fluoro-3-(2-morpholin- 4-yl-acetylamino)-phenylamino]- meth-(E/Z)-ylidene]-4-oxo- thiazolidin-(2-(E or Z))-ylidene]- acetamide | (DMSO-d6, the chief isomer, stored over K₂CO₃): δ = 1.20 (t, 3H); 2.51 (b, 4H); 3.16 (s, 2H); 3.40-3.70 (m, 6H); 4.19 (q, 2H); 6.02 (tt, 1H); 6.95-7.10 (m, 1H); 7.22 (t, 1H); 7.82-8.11 (m, 3H); 9.62 (s, 1H); 10.44 (s, b, 1H) ppm. | | INTT11/INT60/201 |
| 225 | 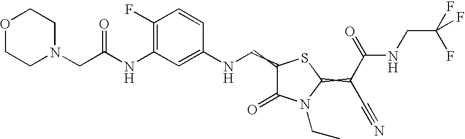 2-cyano-2-[3-ethyl-5-[1-[4-fluoro-3- (2-morpholin-4-yl-acetylamino)- phenylamino]-meth-(E/Z)-ylidene]- 4-oxo-thiazolidin-(2-(E or Z))- ylidene]-N-(2,2,2-trifluoro-ethyl)- acetamide | (DMSO-d6, the chief isomer, stored over K₂CO₃): δ = 1.21 (t, 3H); 2.51 (b, 4H); 3.16 (s, 2H); 3.60 (b, 4H); 3.81-3.99 (m, 2H); 4.19 (q, 2H); 6.95-7.11 (m, 1H); 7.22 (t, 1H); 8.00 (b, 2H); 8.16 (s, b, 1H); 9.63 (s, 1H); 10.47 (s, b, 1H); ppm. | | INTT8/INT60/201 |
| 226 | 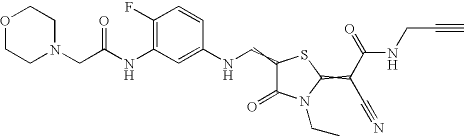 2-cyano-2-[3-ethyl-5-[1-[4-fluoro-3- (2-morpholin-4-yl-acetylamino)- phenylamino]-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 1.20 (t, 3H); 2.51 (b, 4H); 2.97-3.07 (m, 1H); 3.16 (s, 2H); 3.60 (b, 4H); 3.80-3.95 (m, 2H); 4.18 (q, 2H); 6.96-7.12 (m, 1H); 7.22 (t, 1H); 7.85-8.20 (m, 3H); 9.62 (s, 1H); 10.42 (s, b, 1H) ppm. | | INTT9/INT60/201 |
| 227 | 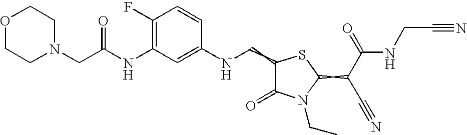 2-cyano-N-cyanomethyl-2-[3-ethyl- 5-[1-[4-fluoro-3-(2-morpholin-4-yl- acetylamino)-phenylamino]-meth- (E/Z)-ylidene]-4-oxo-thiazolidin-(2- (E or Z))-ylidene]-acetamide | (DMSO-d6, the chief isomer, stored over K₂CO₃): δ = 1.20 (t, 3H); 2.51 (b, 4H); 3.16 (s, 2H); 3.60 (b, 4H); 4.12 (d, 2H); 4.19 (q, 2H); 6.97-7.12 (m, 1H); 7.23 (t, 1H); 8.00 (b, 2H); 8.29 (s, b, 1H); 9.63 (s, 1H); 10.49 (s, b, 1H) ppm. | | INTT10/INT60/201 |
| 228 | 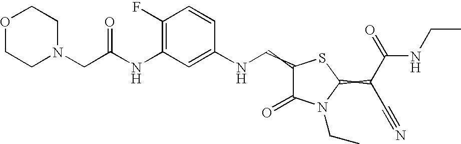 2-cyano-N-ethyl-2-[3-ethyl-5-[1-[4- fluoro-3-(2-morpholin-4-yl- acetylamino)-phenylamino]-meth- (E/Z)-ylidene]-4-oxo-thiazolidin-(2- (E or Z))-ylidene]-acetamide | (DMSO-d6, the chief isomer, stored over K₂CO₃): δ = 1.03 (t, 3H); 1.19 (t, 3H); 2.51 (b, 4H); 3.07-3.23 (m, 4H); 3.60 (b, 4H); 4.18 (q, 2H); 6.90-7.15 (m, 1H); 7.21 (t, 1H); 7.64 (s, b, 1H); 7.70-8.10 (m, 2H); 9.62 (s, 1H); 10.36 (s, b, 1H) ppm. | | INTT7/INT60/201 |
| 229 | 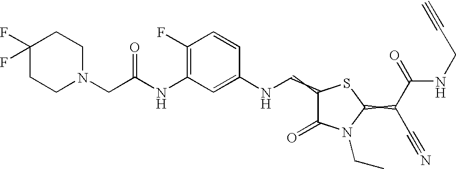 2-cyano-2-[5-[1-{3-[2-(4,4-difluoro- piperidin-1-yl)-acetylamino]-4- fluoro-phenylamino}-meth-(E/Z)- ylidene]-3-ethyl-4-oxo-thiazolidin- (2-(E or Z))-ylidene]-N-prop-2-ynyl- acetamide | (DMSO-d6, the chief isomer, stored over K₂CO₃): δ = 1.17 (t, 3H); 1.85-2.11 (m, 4H); 2.57-2.76 (m, 4H); 2.99 (b, 1H); 3.24 (s, 2H); 3.75-3.95 (m, 2H); 4.17 (q, 2H); 6.88 (b, 1H); 7.13 (t, 1H); 7.74 (m, b, 2H); 8.08 (s, 1H); 9.54 (s, 1H); 10.42 (s, 1H) ppm. | | INTT9/INT62/201 |
| 230 | 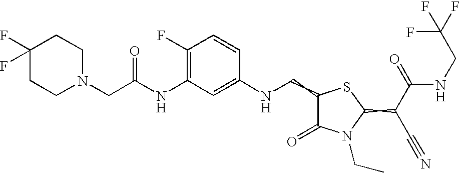 2-cyano-2-[5-[1-{3-[2-(4,4-difluoro- piperidin-1-yl)-acetylamino]-4- fluoro-phenylamino}-meth-(E/Z)- ylidene]-3-ethyl-4-oxo-thiazolidin- (2-(E or Z))-ylidene]-N-(2,2,2- trifluoro-ethyl)-acetamide | (DMSO-d6, the chief isomer, stored over K₂CO₃): δ = 1.21 (t, 3H); 1.85-2.09 (m, 4H); 2.56-2.75 (m, 4H); 3.26 (s, 2H); 3.85-3.95 (m, 2H); 4.19 (q, 2H); 7.05 (b, 1H); 7.21 (t, 1H); 7.93 (b, 1H); 8.01 (s, 1H); 8.14 (b, 1H); 9.61 (s, 1H); 10.47 (s, 1H) ppm. | | INTT8/INT62/201 |
| 231 | 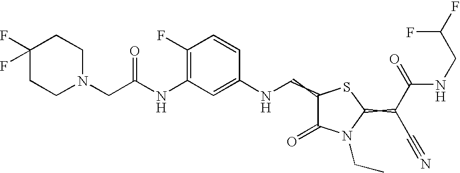 2-cyano-N-(2,2-difluoro-ethyl)-2-[5- [1-{3-[2-(4,4-difluoro-piperidin-1-yl)- acetylamino]-4-fluoro- phenylamino}-meth-(E/Z)-ylidene]- 3-ethyl-4-oxo-thiazolidin-(2-(E or Z))-ylidene]-acetamide | (DMSO-d6, the chief isomer, stored over K₂CO₃): δ = 1.20 (t, 3H); 1.85-2.12 (m, 4H); 2.57-2.73 (m, 4H); 3.26 (s, 2H); 3.42-3.65 (m, 2H); 4.19 (q, 2H); 6.02 (tt, 1H); 6.95-7.20 (b, 1H); 7.21 (t, 1H); 7.75-8.07 (m, 3H); 9.61 (s, 1H); 10.44 (s, b, 1H) ppm. | | INTT11/INT62/201 |
| 232 | 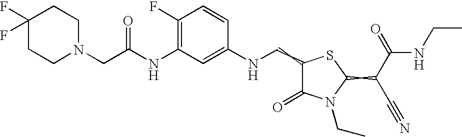 2-cyano-2-[5-[1-{3-[2-(4,4-difluoro- piperidin-1-yl)-acetylamino]-4- fluoro-phenylamino}-meth-(E/Z)- ylidene]-3-ethyl-4-oxo-thiazolidin- (2-(E or Z))-ylidene]-N-ethyl- acetamide | (DMSO-d6, the chief isomer, stored over K₂CO₃): δ = 1.02 (t, 3H); 1.19 (t, 3H); 1.87-2.10 (m, 4H); 2.58-2.75 (m, 4H); 3.16 (m, 2H); 3.25 (s, 2H); 4.17 (q, 2H); 7.01 (b, 1H); 7.19 (t, 1H); 7.56 (b, 1H), 7.87 (b, 1H); 7.96 (s, 1H); 9.60 (s, 1H); 10.35 (s, 1H) ppm. | | INTT7/INT62/201 |
| 233 | 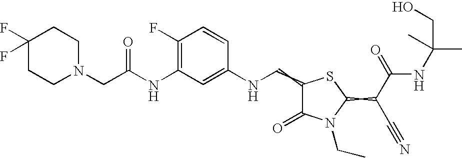 2-cyano-2-[5-[1-{3-[2-(4,4-difluoro- piperidin-1-yl)-acetylamino]-4- fluoro-phenylamino}-meth-(E/Z)- ylidene]-3-ethyl-4-oxo-thiazolidin- (2-(E or Z))-ylidene]-N-(2-hydroxy- 1,1-dimethyl-ethyl)-acetamide | (DMSO-d6, the chief isomer, stored over K₂CO₃): δ = 1.22 (t, 3H); 1.29 (s, 6H); 1.89-2.12 (m, 4H); 2.62-2.76 (m, 4H); 3.29 (s, 2H); 3.37 (d, 2H); 4.19 (q, 2H); 5.18 (t, 1H); 6.63 (s, b, 1H); 7.05 (s, b, 1H); 7.23 (t, 1H); 7.93 (s, b, 1H); 8.01 (s, 1H); 9.64 (s, 1H); 10.39 (s, b, 1H) ppm. | | INTT12/INT62/201 |
| 234 | 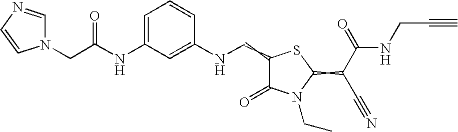 2-cyano-2-[3-ethyl-5-[1-[3-(2- imidazol-1-yl-acetylamino)- phenylamino]-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 1.20 (t, 3H); 2.96-3.08 (m, 1H); 3.82-3.94 (m, 2H); 4.18 (q, 2H); 4.87 (s, 2H); 6.86 (s, 1H); 6.96 (d, 1H); 7.10-7.19 (m, 2H); 7.25 (t, 1H); 7.60 (s, b, 2H); 7.98 (s, 1H); 8.03 (s, b, 1H); 10.33 (s, 1H); 10.41 (s, b, 1H) ppm. | | 200/8 |
| 235 | 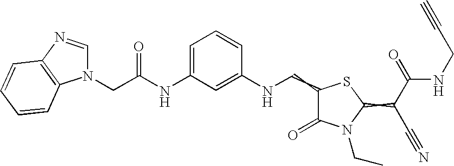 2-[5-[1-[3-(2-benzoimidazol-1-yl- acetylamino)-phenylamino]-meth- (E/Z)-ylidene]-3-ethyl-4-oxo- thiazolidin-(2-(E or Z))-ylidene]-2- cyano-N-prop-2-ynyl-acetamide | (DMSO-d6, the chief isomer, stored over K₂CO₃): δ = 1.19 (t, 3H); 3.01 (b, 1H); 3.82-3.93 (m, 2H); 4.17 (q, 2H); 5.15 (s, 2H); 6.95 (s, b, 1H); 7.12-7.33 (m, 4H); 7.51 (d, 1H); 7.58 (s, b, 1H); 7.64 (d, 1H); 7.99 (b, 2H); 8.20 (s, 1H); 10.40 (s, 1H); 10.48 (s, 1H) ppm. | | 200/8 |
| 236 | 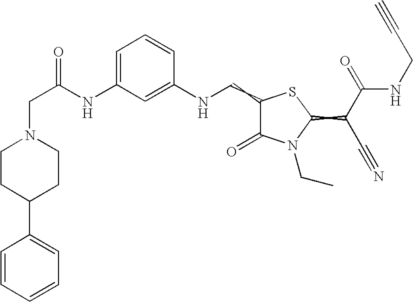 2-cyano-2-[3-ethyl-4-oxo-5-[1-{3-[2- (4-phenyl-piperidin-1-yl)- acetylamino]-phenylamino}-meth- (E/Z)-ylidene]-thiazolidin-(2-(E or Z))-ylidene]-N-prop-2-ynyl- acetamide | (DMSO-d6, the chief isomer, stored over K₂CO₃): δ = 1.20 (t, 3H); 1.63-1.87 (m, 4H); 2.16-2.34 (m, 2H); 2.45-2.55 (m, 1H); 2.95 (d, 2H); 3.01 (b, 1H); 3.12 (s, 2H); 3.81-3.95 (m, 2H); 4.19 (q, 2H); 6.94 (d, 1H); 7.09-7.37 (m, 7H); 7.69 (s, b, 1H); 7.86-8.11 (m, 2H); 9.72 (s, 1H); 10.39 (s, b, 1H) ppm. | | 200/8 |
| 237 | 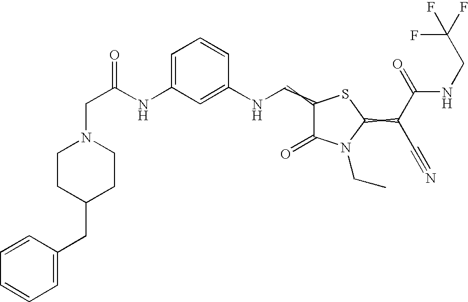 2-[5-[1-{3-[2-(4-benzyl-piperidin-1- yl)-acetylamino]-phenylamino}- meth-(E/Z)-ylidene]-3-ethyl-4-oxo- thiazolidin-(2-(E or Z))-ylidene]-2- cyano-N-(2,2,2-trifluoro-ethyl)- acetamide | (DMSO-d6, the chief isomer, stored over K₂CO₃): δ = 1.15-1.41 (m, 5H); 1.41-1.65 (m, 3H); 2.09 (t, 2H); 2.49-2.60 (m, 2H); 2.85 (d, 2H); 3.06 (s, 2H); 3.87-4.14 (m, 2H); 4.25 (q, 2H); 6.92 (d, 1H); 7.12-7.35 (m, 7H); 7.61 (s, b, 1H); 8.02 (s, b, 1H), 8.11 (s, 1H); 9.64 (s, 1H); 10.50 (s, 1H) ppm. | | 203/8 |
| 238 | 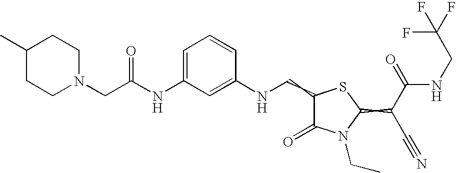 2-cyano-2-[3-ethyl-5-[1-{3-[2-(4- methyl-piperidin-1-yl)-acetylamino]- phenylamino}-meth-(E/Z)-ylidene]- 4-oxo-thiazolidin-(2-(E or Z))- ylidene]-N-(2,2,2-trifluoro-ethyl)- acetamide | (DMSO-d6, the chief isomer, stored over K₂CO₃): δ = 0.91 (d, 3H); 1.16-1.42 (m, 6H); 1.52-1.55 (m, 2H); 2.03-2.20 (m, 2H); 2.83 (d, 2H); 3.09 (s, 2H); 3.88-4.05 (m, 2H); 4.26 (q, 2H); 7.00 (d, 1H); 7.22-7.38 (m, 2H); 7.71 (s, 1H); 8.09 (s, 1H); 8.20 (s, b, 1H); 9.71 (s, 1H); 10.50 (s, b, 1H) ppm. | | 203/8 |
| 239 | 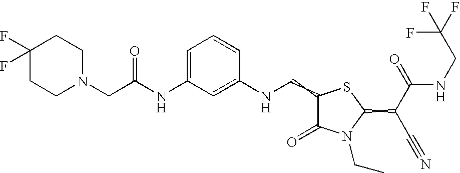 2-cyano-2-[5-[1-{3-[2-(4,4-difluoro- piperidin-1-yl)-acetylamino]- phenylamino}-meth-(E/Z)-ylidene]- 3-ethyl-4-oxo-thiazolidin-(2-(E or Z))-ylidene]-N-(2,2,2-trifluoro- ethyl)-acetamide | (DMSO-d6, the chief isomer, stored over K₂CO₃): δ = 1.21 (t, 3H); 1.91-2.10 (m, 4H); 2.63 (b, 4H); 3.20 (s, 2H); 3.84-4.00 (m, 2H); 4.21 (q, 2H); 6.96 (d, 1H); 7.20-7.32 (m, 2H); 7.71 (s, 1H); 8.05 (d, 1H); 8.20 (t, 1H); 9.79 (s, 1H); 10.50 (d, 1H) ppm. | | 203/8 |
| 240 | 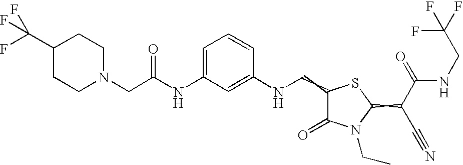 2-cyano-2-[3-ethyl-4-oxo-5-[1-{3-[2- (4-trifluoromethyl-piperidin-1-yl)- acetylamino]-phenylamino}-meth- (E/Z)-ylidene]-thiazolidin-(2-(E or Z))-ylidene]-N-(2,2,2-trifluoro- ethyl)-acetamide | (DMSO-d6, the chief isomer, stored over K₂CO₃): δ = 1.21 (t, 3H); 1.49-1.63 (m, 2H); 1.75 (d, 2H); 2.10-2.31 (m, 2H); 2.90 (d, 2H); 3.11 (s, 2H); 3.85-3.98 (m, 2H); 4.21 (q, 2H); 6.97 (d, 1H); 7.19-7.32 (m, 2H); 7.70 (s, 1H); 8.03 (s, b, 1H); 8.19 (t, 1H); 9.71 (s, 1H); 10.50 (s, b, 1H) ppm. | | 203/8 |
| 241 | 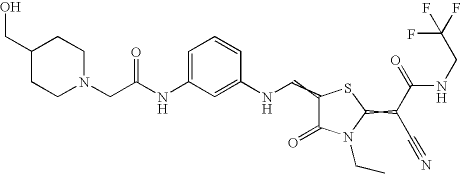 2-cyano-2-[3-ethyl-5-[1-{3-[2-(4- hydroxymethyl-piperidin-1-yl)- acetylamino]-phenylamino}-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 1.13-1.40 (m, 6H); 1.65 (d, 2H); 2.11 (t, 2H); 2.87 (d, 2H); 3.10 (s, 2H); 3.28 (t, 2H); 3.88-4.04 (m, 2H); 4.25 (q, 2H); 4.45 (t, 1H); 6.96 (d, 1H); 7.20-7.36 (m, 2H); 7.70 (s, b, 1H); 8.12 (b, 2H); 9.71 (s, 1H); 10.50 (s, 1H) ppm. | | 203/8 |
| 242 | 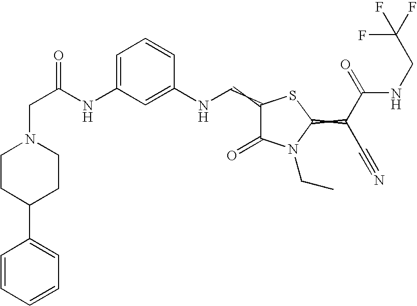 2-cyano-2-[3-ethyl-4-oxo-5-[1-{3-[2- (4-phenyl-piperidin-1-yl)- acetylamino]-phenylamino}-meth- (E/Z)-ylidene]-thiazolidin-(2-(E or Z))-ylidene]-N-(2,2,2-trifluoro- ethyl)-acetamide | (DMSO-d6, the chief isomer, stored over K₂CO₃): δ = 1.21 (t, 3H); 1.69-1.91 (m, 4H); 2.20-2.35 (m, 2H); 2.44-2.60 (m, 1H); 2.99 (d, 2H); 3.15 (s, 2H); 3.85-4.02 (m, 2H); 4.23 (q, 2H); 6.81 (s, b, 1H); 7.12-7.68 (m, 9H); 8.23 (s, b, 1H); 9.65 (s, b, 1H), 10.50 (s, 1H) ppm. | | 203/8 |
| 243 | 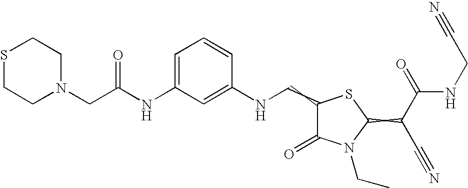 2-cyano-N-cyanomethyl-2-[3-ethyl- 4-oxo-5-[1-[3-(2-thiomorpholin-4-yl- acetylamino)-phenylamino]-meth- (E/Z)-ylidene]-thiazolidin-(2-(E or Z))-ylidene]-acetamide | (DMSO-d6, the chief isomer, stored over K₂CO₃): δ = 1.25 (t, 3H); 2.62-2.87 (m, 8H); 3.17 (s, 2H); 4.16 (d, 2H); 4.23 (q, 2H); 6.93 (b, 1H); 7.19-7.37 (m, 2H); 7.64 (s, b, 1H); 8.02-8.37 (m, 2H); 9.70 (s, 1H); 10.52 (s, b, 1H) ppm. | | 96/8 |
| 244 | 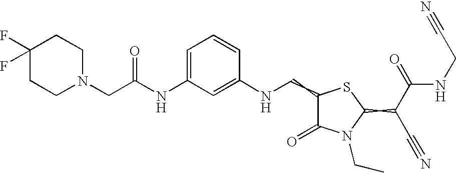 2-cyano-N-cyanomethyl-2-[5-[1-{3- [2-(4,4-difluoro-piperidin-1-yl)- acetylamino]-phenylamino}-meth- (E/Z)-ylidene]-3-ethyl-4-oxo- thiazolidin-(2-(E or Z))-ylidene]- acetamide | (DMSO-d6, the chief isomer, stored over K₂CO₃): δ = 1.26 (t, 3H); 1.93-2.16 (m, 4H); 2.60-2.74 (m, 4H); 3.24 (s, 2H); 4.17 (d, 2H); 4.25 (q, 2H); 6.99 (d, 1H); 7.20-7.40 (m, 2H); 7.72 (s, 1H); 8.09 (s, 1H); 8.31 (s, b, 1H), 9.80 (s, 1H); 10.53 (s, b, 1H) ppm. | | 96/8 |
| 245 | 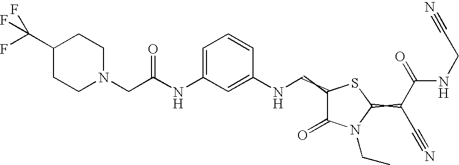 2-cyano-N-cyanomethyl-2-[3-ethyl- 4-oxo-5-[1-{3-[2-(4-trifluoromethyl- piperidin-1-yl)-acetylamino]- phenylamino}-meth-(E/Z)-ylidene]- thiazolidin-(2-(E or Z))-ylidene]- acetamide | (DMSO-d6, the chief isomer, stored over K₂CO₃): δ = 1.25 (t, 3H); 1.50-1.70 (m, 2H); 1.80 (d, 2H); 2.11-2.39 (m, 3H); 2.95 (d, 2H); 3.16 (s, 2H); 4.17 (d, 2H); 4.23 (q, 2H); 7.00 (d, 1H); 7.21-7.38 (m, 2H); 7.73 (s, 1H); 8.09 (s, 1H); 8.34 (t, 1H); 9.77 (s, 1H); 10.52 (s, b, 1H) ppm. | | 96/8 |
| 246 | 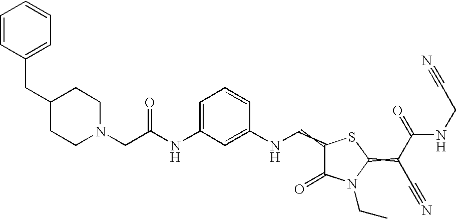 2-[5-[1-{3-[2-(4-benzyl-piperidin-1- yl)-acetylamino]-phenylamino}- meth-(E/Z)-ylidene]-3-ethyl-4-oxo- thiazolidin-(2-(E or Z))-ylidene]-2- cyano-N-cyanomethyl-acetamide | (DMSO-d6, the chief isomer, stored over K₂CO₃): δ = 1.17-1.41 (m, 5H); 1.41-1.63 (m, 3H); 2.08 (t, 2H); 2.42-2.60 (m, 2H); 2.83 (d, 2H); 3.08 (s, 2H); 4.17 (d, 2H); 4.23 (q, 2H); 6.99 (d, 1H); 7.11-7.36 (m, 7H); 7.72 (s, 1H); 8.08 (s, b, 1H); 8.34 (t, 1H); 9.70 (s, 1H); 10.51 (s, b, 1H) ppm. | | 96/8 |
| 247 | 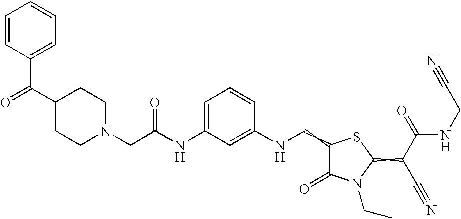 2-[5-[1-{3-[2-(4-Benzoyl-piperidin- 1-yl)-acetylamino]-phenylamino}- meth-(E/Z)-ylidene]-3-ethyl-4-oxo- thiazolidin-(2-(E or Z))-ylidene]-2- cyano-N-cyanomethyl-acetamide | (DMSO-d6, the chief isomer, stored over K₂CO₃): δ = 1.24 (t, 3H); 1.63-1.87 (m, 4H); 2.26-2.43 (m, 2H); 2.85-3.00 (m, 2H); 3.17 (s, 2H); 3.36-3.50 (m, 1H); 4.17 (d, 2H); 4.23 (q, 2H); 7.00 (d, 1H); 7.21-7.40 (m, 2H); 7.56 (t, 2H); 7.65 (t, 1H); 7.75 (s, 1H); 7.99 (d, 2H); 8.10 (s, 1H); 8.32 (s, b, 1H); 9.77 (s, 1H); 10.51 (s, b, 1H) ppm. | 96/8 | |
| 248 | 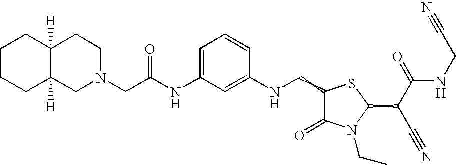 2-cyano-N-cyanomethyl-2-(3-ethyl- 5-{[3-((4aS,8aS)-2-octahydro- isoquinolin-2-yl-acetylamino)- phenylamino]-meth-(E/Z)-ylidene}- 4-oxo-thiazolidin-(2-(E or Z))- ylidene)-acetamide | (DMSO-d6, the chief isomer, stored over K₂CO₃): δ = 1.15-2.05 (m, 15H); 2.15-2.38 (m, 2H); 2.48-2.65 (m, 2H); 2.90-3.20 (m, 2H); 4.16 (d, 2H); 4.24 (q, 2H); 7.01 (d, 1H); 7.20-7.34 (m, 2H); 7.73 (s, 1H); 8.08 (s, b, 1H); 8.34 (t, 1H); 9.62 (s, 1H); 10.52 (s, b, 1H) ppm. | | 96/8 |
| 249 | 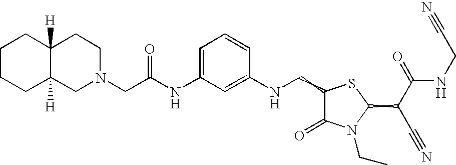 2-cyano-N-cyanomethyl-2-(3-ethyl- 5-{[3-((4aR,8aS)-2-octahydro- isoquinolin-2-yl-acetylamino)- phenylamino]-meth-(E/Z)-ylidene}- 4-oxo-thiazolidin-(2-(E or Z))- ylidene)-acetamide | (DMSO-d6, the chief isomer, stored over K₂CO₃): δ = 0.75-1.13 (m, 3H); 1.10-1.41 (m, 7H); 1.41-1.75 (m, 5H); 1.80 (t, 1H); 2.07-2.23 (m, 1H); 2.72 (d, 1H); 2.88 (d, 1H); 3.10 (s, 2H); 4.17 (d, 2H); 4.24 (q, 2H); 7.00 (d, 1H); 7.20-7.35 (m, 2H); 7.72 (s, 1H); 8.10 (s, b, 1H); 8.34 (t, 1H); 9.72 (s, 1H); 10.52 (s, b, 1H) ppm. | | 96/8 |
| 250 | 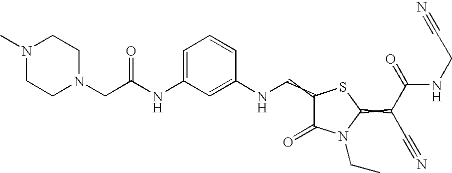 2-cyano-N-cyanomethyl-2-[3-ethyl- 5-[1-{3-[2-(4-methyl-piperazin-1-yl)- acetylamino]-phenylamino}-meth- (E/Z)-ylidene]-4-oxo-thiazolidin-(2- (E or Z))-ylidene]-acetamide | (DMSO-d6, the chief isomer, stored over K₂CO₃): δ = 1.25 (t, 3H); 2.18 (s, 3H); 2.30-2.55 (m, 8H); 3.12 (s, 2H); 4.18 (d, 2H); 4.25 (q, 2H); 6.99 (d, 1H); 7.20-7.36 (m, 2H); 7.71 (s, 1H); 8.10 (s, 1H); 8.34 (s, b, 1H); 9.75 (s, 1H); 10.51 (s, b, 1H) ppm. | | 96/8 |
| 251 | 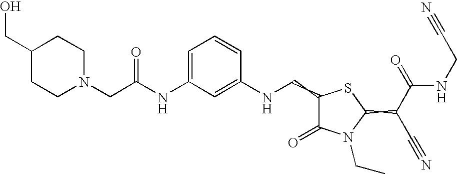 2-cyano-N-cyanomethyl-2-[3-ethyl- 5-[1-{3-[2-(4-hydroxy-piperidin-1- yl)-ethyl]-phenylamino}-meth-(E/Z)- ylidene]-4-oxo-thiazolidin-(2-(E or Z))-ylidene]-acetamide | (DMSO-d6, the chief isomer, stored over K₂CO₃): δ = 1.14-1.40 (m, 6H); 1.65 (d, 2H); 2.10 (t, 2H); 2.85 (d, 2H); 3.10 (s, 2H); 3.26 (t, 2H); 4.16 (d, 2H), 4.24 (q, 2H); 4.44 (t, 1H); 7.00 (d, 1H); 7.21-7.36 (m, 2H); 7.72 (s, 1H); 8.08 (s, b, 1H); 8.33 (t, 1H); 9.71 (s, 1H); 10.52 (s, b, 1H) ppm. | | 96/8 |
| 252 | 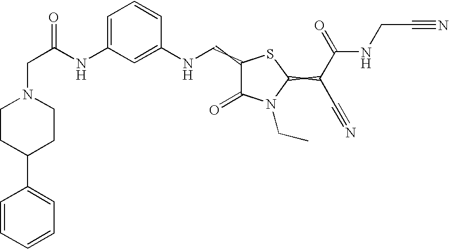 2-cyano-N-cyanomethyl-2-[3-ethyl- 5-[1-{3-[2-(4-hydroxymethyl- piperidin-1-yl)-acetylamino]- phenylamino}-meth-(E/Z)-ylidene]- 4-oxo-thiazolidin-(2-(E or Z))- ylidene]-acetamide | (DMSO-d6, the chief isomer, stored over K₂CO₃): δ = 1.21 (t, 3H); 1.64-1.82 (m, 4H); 2.18-2.32 (m, 2H); 2.47-2.57 (m, 1H); 2.94 (d, 2H); 3.13 (s, 2H); 4.12 (d, 2H); 4.21 (q, 2H); 6.97 (d, 1H); 7.10-7.34 (m, 7H); 7.74 (s, 1H); 8.05 (s, b, 1H), 8.31 (t, 1H); 9.73 (s, 1H); 10.50 (s, b, 1H) ppm. | | 96/8 |
| 253 | 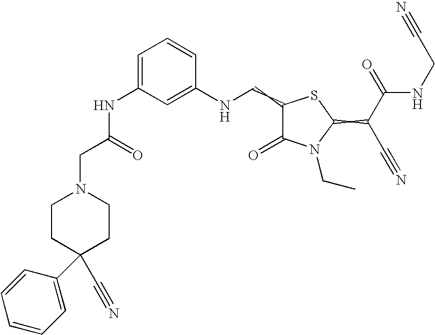 2-cyano-N-cyanomethyl-2-[5-[1-{3- [2-(4-cyano-4-phenyl-piperidin-1- yl)-acetylamino]-phenylamino}- meth-(E/Z)-ylidene]-3-ethyl-4-oxo- thiazolidin-(2-(E or Z))-ylidene]- acetamide | (DMSO-d6, the chief isomer, stored over 1.25 (t, 3H); 2.08-2.29 (m, 4H); 2.55-2.69 (m, 2H); 3.04 (d, 2H); 3.27 (s, 2H); 4.17 (d, 2H); 4.24 (q, 2H); 7.00 (d, 1H); 7.21-7.53 (m, 5H); 7.59 (d, 2H); 7.77 (s, 1H); 8.08 (s, b, 1H); 8.35 (t, 1H); 9.81 (s, 1H); 10.54 (s, b, 1H) ppm. | | 96/8 |
| 254 | 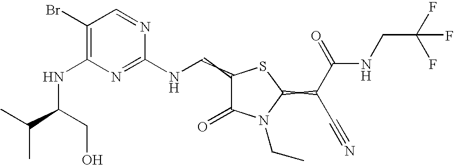 2-[5-[1-[5-bromo-4-((R)-1- hydroxymethyl-2-methyl- propylamino)-pyrimidin-2-ylamino]- meth-(E/Z)-ylidene]-3-ethyl-4-oxo- thiazolidin-(2-(E or Z))-ylidene]-2- cyano-N-(2,2,2-trifluoro-ethyl)- acetamide | (DMSO-d6, the chief isomer, stored over K₂CO₃): δ = 0.97 (d, 3H); 1.01 (d, 3H); 1 .32 (t, 3H); 2.07 (m, 1H); 3.67 (m, 2H); 4.03 (m, 2H); 4.10 (m, 1H); 4.30 (q, 2H); 4.86 (s, b, 1H), 6.60 (s, b, 1H); 8.25 (s, 1H); 8.35 (s, b, 1H); 8.62 (s, b, 1H), 11.09 (s, b, 1H) ppm. | | INTT8 + INT67/201 |
| 255 | 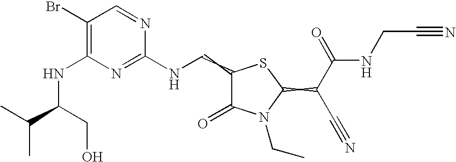 2-[5-[1-[5-bromo-4-((R)-1- hydroxymethyl-2-methyl- propylamino)-pyrimidin-2-ylamino]- meth-(E/Z)-ylidene]-3-ethyl-4-oxo- thiazolidin-(2-(E or Z))-ylidene]-2- cyano-N-cyanomethyl-acetamide | (DMSO-d6, the chief isomer, stored over K₂CO₃): δ = 0.90 (d, 3H); 0.96 (d, 3H); 1.26 (t, 3H); 1.94-2.04 (m, 1H); 3.55-3.69 (m, 2H); 4.05 (m, 1H); 4.18 (d, 2H); 4.23 (q, 2H); 4.79 (t, 1H); 6.55 (d, 1H); 8.19 (s, 1H); 8.41 (t, 1H); 8.57 (s, b, 1H); 11.04 (s, b, 1H) ppm. | | INTT10 + INT67/201 |
| 256 | 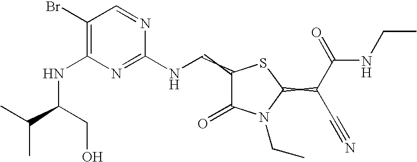 2-[5-[1-[5-bromo-4-((R)-1- hydroxymethyl-2-methyl- propylamino)-pyrimidin-2-ylamino]- meth-(E/Z)-ylidene]-3-ethyl-4-oxo- thiazolidin-(2-(E or Z))-ylidene]-2- cyano-N-ethyl-acetamide | (DMSO-d6, the chief isomer, stored over K₂CO₃): δ = 0.88 (d, 3H); 0.92 (d, 3H); 1.06 (t, 3H); 1.23 (t, 3H); 1.90-2.01 (m, 1H); 3.12-3.22 (m, 2H); 3.51-3.66 (m, 2H); 4.03 (m, 1H); 4.20 (q, 2H); 4.77 (t, 1H); 6.50 (s, b, 1H); 7.75 (s, b, 1H); 8.15 (s, 1H); 8.50 (s, 1H); 10.90 (s, 1H) ppm. | | INTT7 + INT67/201 |
| 257 | 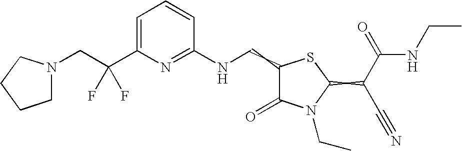 2-cyano-2-[5-[1-[6-(1,1-difluoro-2- pyrrolidin-1-yl-ethyl)-pyridin-2- ylamino]-meth-(E/Z)-ylidene]-3- ethyl-4-oxo-thiazolidin-(2-(E or Z))- ylidene]-N-ethyl-acetamide | (DMSO-d6, the chief isomer, stored over K₂CO₃): δ = 1.03 (t, 3H); 1.21 (t, 3H); 1.57 (b, 4H); 2.50 (b, 4H); 3.10-3.35 (m, 4H); 4.18 (q, 2H); 7.15 (d, 1H); 7.28 (d, 1H); 7.76 (t, 1H); 7.85 (t, 1H); 8.55 (d, 1H); 10.46 (d, 1H) ppm. | | INTA23/1 |
| 258 | 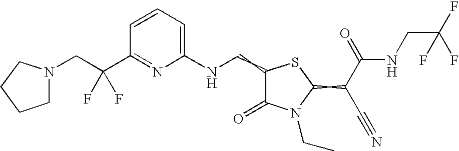 2-cyano-2-[5-[1-[6-(1,1-difluoro-2- pyrrolidin-1-yl-ethyl)-pyridin-2- ylamino]-meth-(E/Z)-ylidene]-3- ethyl-4-oxo-thiazolidin-(2-(E or Z))- ylidene]-N-(2,2,2-trifluoro-ethyl)- acetamide | (DMSO-d6, the chief isomer, stored over K₂CO₃): δ = 1.22 (t, 3H); 1.57 (b, 4H); 2.50 (b, 4H); 3.20-3.32 (m, 2H); 3.85-3.99 (m, 2H); 4.20 (q, 2H); 7.15 (d, 1H); 7.30 (d, 1H); 7.86 (t, 1H); 8.27 (t, 1H); 8.61 (s, b, 1H); 10.97 (s, b, 1H) ppm. | | INTA23/1 |
| 259 | 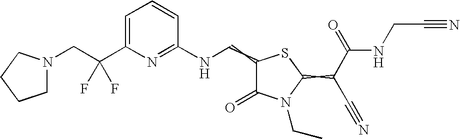 2-cyano-N-cyanomethyl-2-[5-[1-[6- (1,1-difluoro-2-pyrrolidin-1-yl-ethyl)- pyridin-2-ylamino]-meth-(E/Z)- ylidene]-3-ethyl-4-oxo-thiazolidin- (2-(E or Z))-ylidene]-acetamide | (DMSO-d6, the chief isomer, stored over K₂CO₃): δ = 1.22 (t, 3H); 1.58 (b, 4H); 2.52 (b, 4H); 3.20-3.35 (m, 2H); 4.13 (d, 2H); 4.20 (q, 2H); 7.27 (d, 1H); 7.31 (d, 1H); 7.88 (t, 1H); 8.41 (t, 1H); 8.61 (s, b, 1H), 11.00 (s, b, 1H) ppm. | INTA23/1 | |
| 260 | 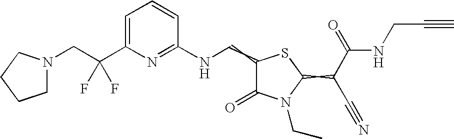 2-cyano-2-[5-[1-[6-(1,1-difluoro-2- pyrrolidin-1-yl-ethyl)-pyridin-2- ylamino]-meth-(E/Z)-ylidene]-3- ethyl-4-oxo-thiazolidin-(2-(E or Z))- ylidene]-N-prop-2-ynyl-acetamide | (DMSO-d6, the chief isomer, stored over K₂CO₃): δ = 1.21 (t, 3H); 1.57 (b, 4H); 2.50 (b, 4H); 3.03 (b, 1H); 3.16-3.36 (m, 2H); 3.83-3.95 (m, 2H); 4.19 (q, 2H); 7.15 (d, 1H); 7.29 (d, 1H); 7.85 (t, 1H); 8.16 (t, 1H); 8.58 (d, 1H); 10.92 (d, 1H) ppm. | INTA23/1 | |
| 261 | 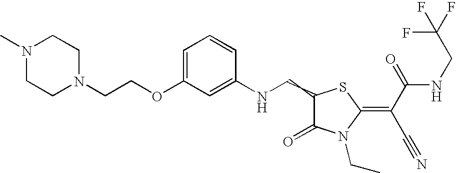 2-cyano-2-[3-ethyl-5-[1-{3-[2-(4- methyl-piperazin-1-yl)-ethoxy]- phenylamino}-meth-(E/Z)-ylidene]- 4-oxo-thiazolidin-(2-(E or Z))- ylidene]-N-(2,2,2-trifluoro-ethyl)- acetamide | (Methanol): δ = 1.30 (m, 3H); 2.71 (s, 3H); 2.88 (m, 6H, wide); 3.10 (m, 4H); 3.99 (m, 2H); 4.18 (m, 2H); 4.33 (m, 2H); 6.70 (dd, 1H); 6.80 (m, 2H); 7.28 (t, 1H); 8.19 (s, 1H) ppm. | 538.60/539 | INTT8/INT85/5 |
| 262 | 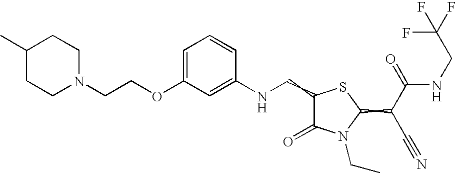 2-cyano-2-[3-ethyl-5-[1-{3-[2-(4- methyl-piperidin-1-yl)-ethoxy]- phenylamino}-meth-(E/Z)-ylidene]- 4-oxo-thiazolidin-(2-(E or Z))- ylidene]-N-(2,2,2-trifluoro-ethyl)- acetamide | (DMSO-d6, the chief isomer, stored over K₂CO₃): δ = 0.85 (d, 3H); 1.19 (m, 6H, wide); 3.16 (m, 2H); 2.00 (m, 2H); 2.63 (m, 2H); 2.88 (m, 2H); 3.90 (m, 2H); 4.07 (m, 2H); 4.22 (m, 2H); 6.61 (dd, 1H); 6.88 (m, 2H); 7.20 (t, 1H); 8.12 (m, 2H); 10.25 (d, 1H) ppm. | 537.61/538 | INTT8/INT87/5 |
| 263 | 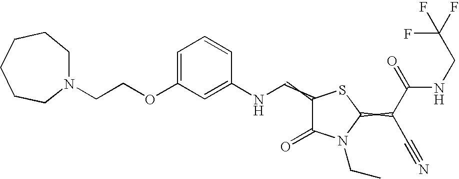 2-[5-[1-[3-(2-azepan-1-yl-ethoxy)- phenylamino]-meth-(E/Z)-ylidene]- 3-ethyl-4-oxo-thiazolidin-(2-(E or Z))-lidene]-2-cyano-N-(2,2,2- trifluoro-ethyl)-acetamide | (DMSO-d6, the chief isomer, stored over K₂CO₃): δ = 1.22 (m, 3H); 1.52 (m, 8H); 2.65 (m, 4H); 2.82 (m, 2H); 3.90 (m, 2H); 4.01 (m, 2H); 4.20 (m, 2H); 6.61 (dd, 1H); 6.83 (m, 2H); 7.19 (m, 1H); 8.08 (s, 1H), 8.16 (t, 1H); 10.29 (s, 1H) ppm. | 537.61/538 | INTT8/INT89/5 |
| 264 | 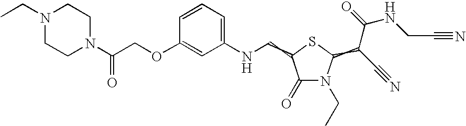 2-cyano-N-cyanomethyl-2-[3-ethyl- 5-[1-{3-[2-(4-ethyl-piperazin-1-yl)-2- oxo-ethoxy]-phenylamino}-meth- (E/Z)-ylidene]-4-oxo-thiazolidin-(2- (E or Z))-ylidene]-acetamide | (DMSO-d6, the chief isomer, stored over K₂CO₃): δ = 0.98 (t, 3H); 1.20 (m, 3H); 2.31 (m, 6H); 3.40 (m, 4H); 4.12 (d, 2H); 4.20 (m, 2H); 4.80 (s, 2H); 6.60 (dd, 2H); 6.81 (d, 1H); 6.88 (dd, 1H); 7.20 (t, 1H); 8.10 (s, 1H); 8.29 (t, 1H); 10.21 (s, 1H) ppm. | 523.62/524 | INTA26/204 |
| 265 | 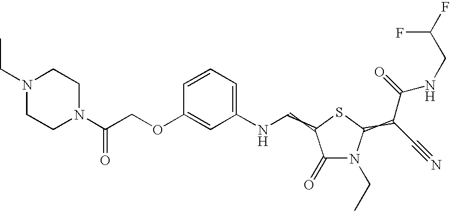 2-cyano-N-(2,2-difluoro-ethyl)-2-[3- ethyl-5-[1-{3-[2-(4-ethyl-piperazin- 1-yl)-2-oxo-ethoxy]-phenylamino}- meth-(E/Z)-ylidene]-4-oxo- thiazolidin-(2-(E or Z))-ylidene]- acetamide | (DMSO-d6, the chief isomer, stored over K₂CO₃): δ = 0.99 (t, 3H); 1.21 (t, 3H); 2.30 (m, 6H); 3.41 (m, 4H); 3.55 (m, 2H); 4.20 (m, 2H); 4.79 (s, 2H); 6.03 (tt, 1H); 6.58 (dd, 1H), 6.81 (m, 2H); 7.19 (t, 1H); 7.80 (m, 1H); 8.10 (s, 1H); 9.95 (s, 1H) ppm. | 548.62/549 | INTA26/204 |
| 266 | 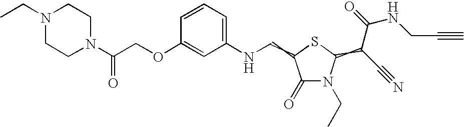 2-cyano-2-[3-ethyl-5-[1-{3-[2-(4- ethyl-piperazin-1-yl)-2-oxo-ethoxy]- phenylamino}-meth-(E/Z)-ylidene]- 4-oxo-thiazolidin-(2-(E or Z))- ylidene]-N-prop-2-ynyl-acetamide | (DMSO-d6, the chief isomer, stored over K₂CO₃): δ = 0.99 (t, 3H); 1.21 (t, 3H); 2.31 (m, 6H); 3.41 (m, 4H); 3.89 (m, 2H); 4.17 (m, 2H); 4.79 (s, 2H); 6.57 (dd, 1H); 6.83 (m, 2H); 7.20 (t, 1H); 8.07 (m, 1H); 10.20 (s, 1H) ppm. | 522.63/523 | INTA26/204 |
| 267 | 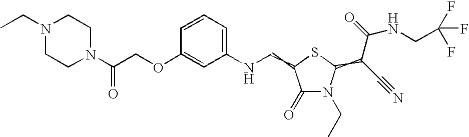 2-cyano-2-[3-ethyl-5-[1-{3-[2-(4- ethyl-piperazin-1-yl)-2-oxo-ethoxy]- phenylamino}-meth-(E/Z)-ylidene]- 4-oxo-thiazolidin-(2-(E or Z))- ylidene]-N-(2,2,2-trifluoro-ethyl)- acetamide | (DMSO-d6, the chief isomer, stored over K₂CO₃): δ = 0.99 (t, 3H); 1.23 (m, 3H); 2.31 (m, 6H); 3.41 (m, 4H); 3.90 (m, 2H); 4.20 (m, 2H); 4.79 (s, 2H); 6.57 (dd, 1H), 6.80 (s, 1H); 6.83 (d, 1H); 7.21 (t, 1H); 8.09 (m, 2H); 10.10 (s, 1H) ppm. | 566.61/567 | INTA26/204 |
| 268 | 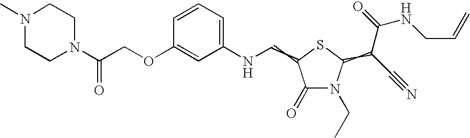 N-Allyl-2-cyano-2-[3-ethyl-5-[1-{3- [2-(4-methyl-piperazin-1-yl)-2-oxo- ethoxy]-phenylamino}-meth-(E/Z)- ylidene]-4-oxo-thiazolidin-(2-(E or Z))-ylidene]-acetamide | (DMSO-d6, the chief isomer, stored over K₂CO₃): δ = 1.21 (t, 3H); 2.18 (s, 3H); 2.21 (m, 2H); 2.30 (m, 2H); 3.42 (m, 4H); 4.20 (m, 2H); 4.81 (s, 2H); 5.09 (m, 2H); 5.81 (m, 1H); 6.60 (dd, 1H); 6.80 (s, 1H); 6.88 (d, 1H); 7.20 (d, 1H); 7.83 (t, 1H); 8.08 (d, 1H); 10.20 (d, 1H) ppm. | 510.62/511 | INTA27/204 |
| 269 | 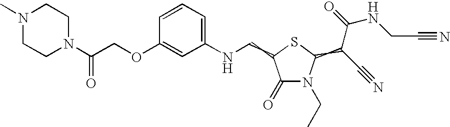 2-cyano-N-cyanomethyl-2-[3-ethyl- 5-[1-{3-[2-(4-methyl-piperazin-1-yl)- 2-oxo-ethoxy]-phenylamino}-meth- (E/Z)-ylidene]-4-oxo-thiazolidin-(2- (E or Z))-ylidene]-acetamide | (DMSO-d6, the chief isomer, stored over K₂CO₃): δ = 1.20 (t, 3H); 2.18 (s, 3H); 2.21 (m, 2H); 2.40 (m, 2H); 3.39 (m, 4H); 4.12 (d, 2H); 4.20 (m, 2H); 4.80 (s, 2H), 6.58 (dd, 1H); 6.82 (s, 1H); 6.87 (d, 1H); 7.21 (t, 1H); 8.10 (s, 1H); 8.31 (t, 1H); 10.30 (s, 1H) ppm. | 509.59/510 | INTA27/204 |
| 270 | 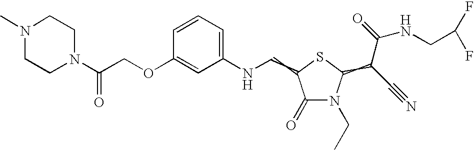 2-cyano-N-(2,2-difluoro-ethyl)-2-[3- ethyl-5-[1-{3-[2-(4-methyl-iperazin- 1-yl)-2-oxo-ethoxy]-phenylamino}- meth-(E/Z)-ylidene]-4-oxo- thiazolidin-(2-(E or Z))-ylidene]- acetamide | (DMSO-d6, the chief isomer, stored over K₂CO₃): δ = 1.20 (t, 3H); 2.18 (s, 3H); 2.21 (m, 2H); 2.32 (m, 2H); 3.40 (m, 4H); 3.52 (m, 2H); 4.20 (m, 2H); 4.79 (s, 2H); 6.03 (tt, 1H); 6.59 (dd, 1H); 6.81 (s, 1H); 6.86 (d, 1H); 7.19 (t, 1H); 7.92 (m, 1H); 8.08 (m, 1H); 10.31 (d, 1H) ppm. | 534.59/535 | INTA27/204 |
| 271 | 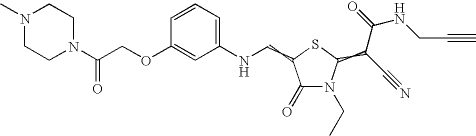 2-cyano-2-[3-ethyl-5-[1-{3-[2-(4- methyl-piperazin-1-yl)-2-oxo- ethoxy]-phenylamino}-meth-(E/Z)- ylidene]-4-oxo-thiazolidin-(2-(E or Z))-ylidene]-N-prop-2-ynyl- acetamide | (DMSO-d6, the chief isomer, stored over K₂CO₃): δ = 1.21 (t, 3H); 2.19 (s, 3H); 2.22 (m, 2H); 2.31 (m, 2H); 3.02 (m, 1H); 3.39 (m, 4H); 3.88 (m, 2H); 4.20 (m, 2H); 4.80 (s, 2H); 6.58 (dd, 1H); 6.80 (s, 1H); 6.83 (d, 1H); 7.19 (t, 3H); 8.08 (s, 2H); 10.25 (s, 1H) ppm. | 508.60/509 | INTA27/204 |
| 272 | 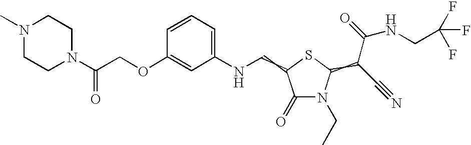 2-cyano-2-[3-ethyl-5-[1-{3-[2-(4- methyl-piperazin-1-yl)-2-oxo- ethoxy]-phenylamino}-meth-(E/Z)- ylidene]-4-oxo-thiazolidin-(2-(E or Z))-ylidene]-N-(2,2,2-trifluoro- ethyl)-acetamide | (DMSO-d6, the chief isomer, stored over K₂CO₃): δ = 1.22 (t, 3H); 2.18 (s, 3H); 2.22 (m, 2H); 2.31 (m, 2H); 3.41 (m, 4H); 3.91 (m, 2H); 4.20 (m, 2H); 4.80 (s, 2H); 6.61 (dd, 1H); 6.80 (s, 1H); 6.88 (d, 1H); 7.21 (t, 1H), 8.10 (s, 1H); 8.18 (m, 1H); 10.21 (s, 1H) ppm. | 552.58/553 | INTA27/204 |
| 273 | 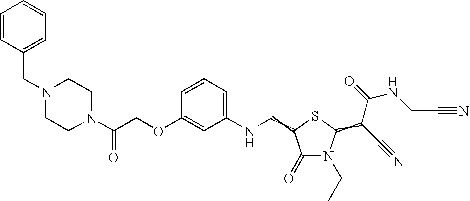 2-[5-[1-{3-[2-(4-benzyl-piperazin-1- yl)-2-oxo-ethoxy]-phenylamino}- meth-(E/Z)-ylidene]-3-ethyl-4-oxo- thiazolidin-(2-(E or Z))-ylidene]-2- cyano-N-cyanomethyl-acetamide | (DMSO-d6, the chief isomer, stored over K₂CO₃): δ = 1.21 (t, 3H); 2.30 (m, 2H); 2.39 (m, 2H); 3.41 (m, 4H); 3.49 (s, 2H); 4.11 (d, 2H); 4.20 (m, 2H); 4.79 (s, 2H); 6.60 (dd, 1H); 6.80 (s, 1H); 6.85 (d, 1H); 7.21 (t, 1H); 7.29 (m, 5H); 8.09 (s, 1H); 8.31 (t, 1H); 10.30 (s, 1H) ppm. | 585.69/586 | INTA23/204 |
| 274 | 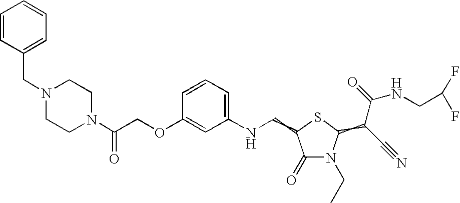 2-[5-[1-{3-[2-(4-benzyl-piperazin-1- yl)-2-oxo-ethoxy]-phenylamino}- meth-(E/Z)-ylidene]-3-ethyl-4-oxo- thiazolidin-(2-(E or Z))-ylidene]-2- cyano-N-(2,2-difluoro-ethyl)- acetamide | (DMSO-d6, the chief isomer, stored over K₂CO₃): δ = 1.21 (t, 3H); 2.29 (m, 2H); 2.49 (m, 2H); 3.42 (m, 4H); 3.48 (s, 2H); 3.57 (m, 2H), 4.20 (m, 2H); 4.80 (s, 2H); 6.02 (tt, 1H); 6.48 (dd, 1H), 6.80 (s, 1H); 6.86 (d, 1H); 7.20 (t, 1H); 7.29 (m, 5H); 7.92 (t, 1H); 8.08 (d, 1H); 10.27 (d, 1H) ppm. | 610.69/611 | INTA23/204 |
| 275 | 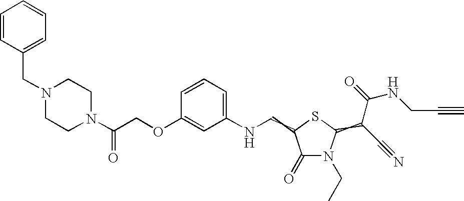 2-[5-[1-{3-[2-(4-benzyl-piperazin-1- yl)-2-oxo-ethoxy]-phenylamino}- meth-(E/Z)-ylidene]-3-ethyl-4-oxo- thiazolidin-(2-(E or Z))-ylidene]-2- cyano-N-prop-2-ynyl-acetamide | (DMSO-d6, the chief isomer, stored over K₂CO₃): δ = 1.21 (t, 3H); 2.28 (m, 2H); 2.39 (m, 2H); 3.02 (m, 1H); 3.47 (m, 4H); 3.49 (s, 2H); 3.90 (m, 2H), 4.20 (m, 2H); 4.79 (s, 2H); 6.58 (dd, 1H); 6.80 (s, 1H); 6.86 (d, 1H); 7.18 (t, 1H); 7.29 (m, 5H); 8.07 (m, 2H); 10.21 (d, 1H) ppm. | 584.70/585 | INTA23/204 |
| 276 | 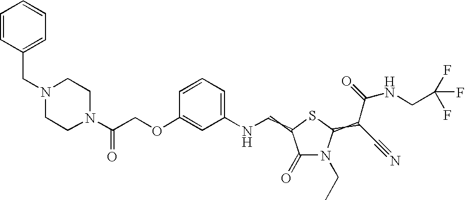 2-[5-[1-{3-[2-(4-benzyl-piperazin-1- yl)-2-oxo-ethoxy]-phenylamino}- meth-(E/Z)-ylidene]-3-ethyl-4-oxo- thiazolidin-(2-(E or Z))-ylidene]-2- cyano-N-(2,2,2-trifluoro-ethyl)- acetamide | (CDCl₃, chief isomer): δ = 1.41 (m, 3H); 3.08 (m, 4H); 3.66 (m, 4H); 3.80 (m, 2H); 4.00 (m, 2H); 4.38 (m, 2H); 4.74 (s, 2H); 6.65 (m, 4H); 7.33 (m, 4H); 7.62 (m, 2H); 8.07 (d, 1H); 10.50 (d, 1H) ppm. | 470.60/471 | INTA28/1 |
| 277 | 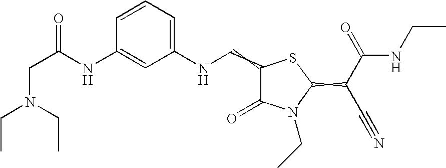 2-cyano-2-[5-[1-[3-(2-diethylamino- acetylamino)-phenylamino]-meth- (E/Z)-ylidene]-3-ethyl-4-oxo- thiazolidin-(2-(E or Z))-ylidene]-N- ethyl-acetamide | (DMSO-d6, the chief isomer, stored over K₂CO₃): δ = 1.02 (t, 6H); 1.07 (t, 3H); 1.24 (t, 3H); 2.60 (q, 4H); 3.15 (s, 2H); 3.16-3.23 (m, 2H); 4.22 (q, 2H); 6.95-6.97 (m, 1H); 7.22-7.32 (m, 2H); 7.69 (t, 1H); 7.72 (1H); 8.02 (1H); 9.68 (s, 1H); 10.35 (1H) ppm. | 470.60/471 | INTA28/1 |
| 278 | 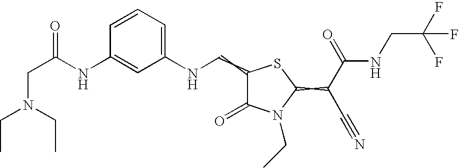 2-cyano-2-[5-[1-[3-(2-diethyl- amino-acetylamino)-phenylamino]- meth-(E/Z)-ylidene]-3-ethyl-4-oxo- thiazolidin-(2-(E or Z))-ylidene]-N- (2,2,2-trifluoro-ethyl)-acetamide | (DMSO-d6, the chief isomer, stored over K₂CO₃): δ = 1.02 (t, 6H); 1.24 (t, 3H); 2.60 (q, 4H); 3.15 (s, 2H); 3.90-3.99 (m, 2H); 4.23 (q, 2H); 6.97-6.99 (m, 1H); 7.23-7.33 (m, 2H); 7.74 (s, 1H); 8.08 (1H); 8.20 (t, 1H); 9.69 (s, 1H); 10.74 (1H) ppm. | 524.57/525 | INTA28/1 |
| 279 | 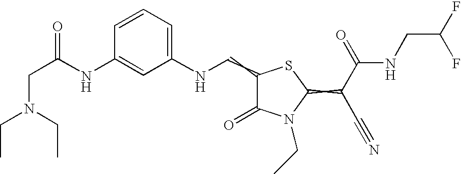 2-cyano-2-[5-[1-[3-(2-diethyl- amino-acetylamino)-phenylamino]- meth-(E/Z)-ylidene]-3-ethyl-4-oxo- thiazolidin-(2-(E or Z))-ylidene]-N- (2,2-difluoro-ethyl)-acetamide | (DMSO-d6, the chief isomer, stored over K₂CO₃): δ = 1.02 (t, 6H); 1.25 (t, 3H); 2.60 (q, 4H); 3.15 (s, 2H); 3.58 (tt, 2H); 4.23 (q, 2H); 6.05 (tt, 1H); 6.97-6.99 (m, 1H); 7.23-7.30 (m, 2H); 7.74 (s, 1H); 7.95 (t, 1H); 8.05-8.07 (1H); 9.70 (s, 1H); 10.43-10.46 (1H) ppm. | 506.58/507 | INTA28/1 |
| 280 | 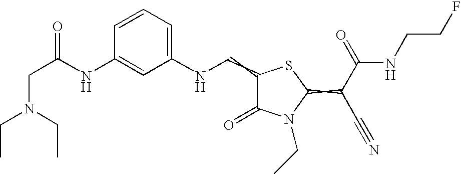 2-cyano-2-[5-[1-[3-(2-diethyl- amino-acetylamino)-phenylamino]- meth-(E/Z)-ylidene]-3-ethyl-4-oxo- thiazolidin-(2-(E or Z))-ylidene]-N- (2-fluoro-ethyl)-acetamide | (DMSO-d6, the chief isomer stored over K₂CO₃): δ = 1.02 (t, 6H); 1.24 (t, 3H); 2.60 (q, 4H); 3.15 (s, 2H); 3.44-3.54 (2q, 2H); 4.22 (q, 2H); 4.42 (t, 1H); 4.54 (t, 1H) 6.05 (tt, 1H); 6.96-6.98 (m, 1H); 7.23-7.32 (m, 2H); 7.73 (s, 1H); 7.78 (t, 1H); 8.03-8.06 (1H); 9.70 (s, 1H); 10.39-10.42 (1H) ppm. | 488.59/489 | INTA28/1 |
| 281 | 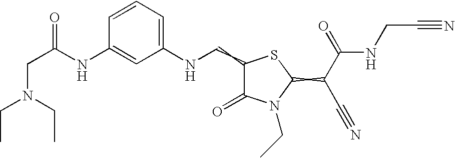 2-cyano-N-cyanomethyl-2-[5-[1-[3- (2-diethylamino-acetylamino)- phenylamino]-meth-(E/Z)-ylidene]- 3-ethyl-4-oxo-thiazolidin-(2(E or Z))-ylidene]-acetamide | (DMSO-d6, the chief isomer, stored over K₂CO₃): δ = 1.02 (t, 6H); 1.25 (t, 3H); 2.60 (q, 4H); 3.15 (s, 2H); 3.44-3.54 (2q, 2H); 4.15 (d, 2H), 4.22 (q, 1H); 6.98-7.00 (m, 1H); 7.23-7.34 (m, 2H); 7.75 (s, 1H); 8.07-8.10 (1H); 8.34 (t, 1H); 9.70 (s, 1H); 10.49-10.52 (1H) ppm. | 481.58/459 | INTA28/1 |
| 282 | 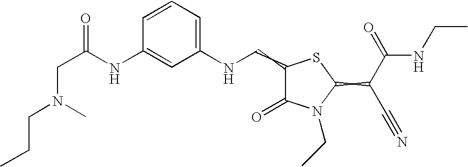 2-cyano-N-ethyl-2-[3-ethyl-5-[1-{3- [2-(methyl-propyl-amino)-acetyl- amino]-phenylamino}-meth-(E/Z)- ylidene]-4-oxo-thiazolidin-(2-(E or Z))-ylidene]-acetamide | (DMSO-d6, the chief isomer, stored over K₂CO₃): δ = 0.87 (t, 3H); 1.07 (t, 3H); 1.24 (t, 3H); 1.43-1.53 (m, 2H); 2.29 (s, 3H); 2.39 (t, 2H); 3.12 (s, 2H); 3.16-3.23 (m, 2H); 4.21 (q, 2H); 6.95-6.97 (m, 1H); 7.22-7.29 (m, 2H); 7.68 (t, 1H); 7.72 (1H); 8.01 (1H); 9.68 (s, 1H); 10.37 (1H) ppm. | 470.60/471 | INTA29/1 |
| 283 | 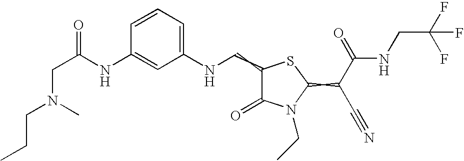 2-cyano-2-[3-ethyl-5-[1-{3-[2- (methyl-propyl-amino)-acetyl- amino]-phenylamino}-meth-(E/Z)- ylidene]-4-oxo-thiazolidin-(2-(E or Z))-ylidene]-N-(2,2,2-tri- fluoro-ethyl)-acetamide | (DMSO-d6, the chief isomer, stored over K₂CO₃): δ = 0.88 (t, 3H); 1.25 (t, 3H); 1.43-1.52 (m, 2H); 2.29 (s, 3H); 2.40 (t, 2H); 3.12 (s, 2H); 3.90-3.99 (m, 2H); 4.23 (q, 2H); 6.97-6.99 (m, 1H); 7.23-7.30 (m, 2H); 7.75 (s, 1H); 8.05-8.07 (1H); 8.20 (t, 1H); 9.69 (s, 1H); 10.47-10.49 (1H) ppm. | 524.57/525 | INTA29/1 |
| 284 | 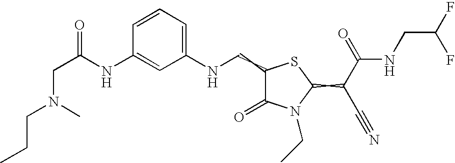 2-cyano-2-[3-ethyl-5-[1-{3-[2- (methyl-propyl-amino)-acetyl- amino]-phenylamino}-meth-(E/Z)- ylidene]-4-oxo-thiazolidin-(2-(E or Z))-ylidene]-N-(2,2-difluoro-ethyl)- acetamide | (DMSO-d6, the chief isomer, stored over K₂CO₃): δ = 0.88 (t, 3H); 1.25 (t, 3H); 1.43-1.52 (m, 2H); 2.29 (s, 3H); 2.39 (t, 2H); 3.12 (s, 2H); 3.58 (tt, 2H); 4.23 (q, 2H); 6.05 (tt, 1H); 6.96-6.98 (m, 1H); 7.23-7.29 (m, 2H); 7.74 (s, 1H), 7.95 (t, 1H); 8.05 (1H); 9.68 (s, 1H); 10.45 (1H) ppm. | 506.58/507 | INTA29/1 |
| 285 | 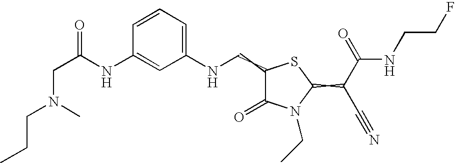 2-cyano-2-[3-ethyl-5-[1-{3-[2- (methyl-propyl-amino)-acetyl- amino]-phenylamino}-meth-(E/Z)- ylidene]-4-oxo-thiazolidin-(2-(E or Z))-ylidene]-N-(2-fluoro-ethyl)- acetamide | (DMSO-d6, the chief isomer, stored over K₂CO₃): δ = 0.88 (t, 3H); 1.25 (t, 3H); 1.43-1.52 (m, 2H); 2.29 (s, 3H); 2.40 (t, 2H); 3.12 (s, 2H); 3.44-3.54 (2q, 2H); 4.23 (q, 2H); 4.41-4.55 (2t, 2H); 6.96-6.98 (m, 1H); 7.22-7.29 (m, 2H); 7.74 (s, 1H); 7.80 (t, 1H); 8.01-8.04 (1H); 9.69 (s, 1H); 10.40-10.43 (1H) ppm. | 488.59/489 | INTA29/1 |
| 286 | 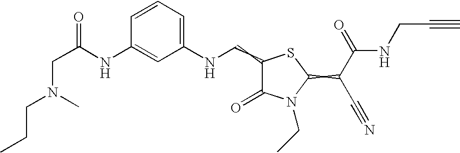 2-cyano-2-[3-ethyl-5-[1-{3-[2- (methyl-propyl-amino)-acetyl- amino]-phenylamino}-meth-(E/Z)- ylidene]-4-oxo-thiazolidin-(2-(E or Z))-ylidene]-N-prop-2-ynyl- acetamide | (DMSO-d6, the chief isomer, stored over K₂CO₃): δ = 0.88 (t, 3H); 1.24 (t, 3H); 1.42-1.52 (m, 2H); 2.29 (s, 3H); 2.40 (t, 2H); 3.06 (m, 1H); 3.12 (s, 2H); 3.91-3.93 (m, 2H), 4.22 (q, 2H); 6.96-6.98 (m, 1H); 7.23-7.29 (m, 2H); 7.74 (s, 1H); 8.05-8.08 (1H); 8.01-8.04 (m, 2H); 9.68 (s, 1H); 10.44 (1H) ppm. | 480.59/481 | INTA29/1 |
| 287 | 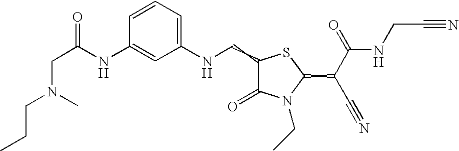 2-cyano-N-cyanomethyl-2-[3-ethyl- 5-[1-{3-[2-(methyl-propyl-amino)- acetylamino]-phenylamino}-meth- (E/Z)-ylidene]-4-oxo-thiazolidin-(2- (E or Z))-ylidene]-acet-amide | (DMSO-d6, the chief isomer, stored over K₂CO₃): δ = 0.88 (t, 3H); 1.25 (t, 3H); 1.43-1.52 (m, 2H); 2.29 (s, 3H); 2.40 (t, 2H); 3.12 (s, 2H); 4.15 (d, 2H); 4.23 (q, 2H); 6.97-6.99 (m, 1H); 7.23-7.30 (m, 2H); 7.75 (s, 1H); 8.08 (1H); 8.34 (t, 1H); 9.69 (s, 1H); 10.51 (1H) ppm. | 481.58/459 | INTA29/1 |
| 288 | 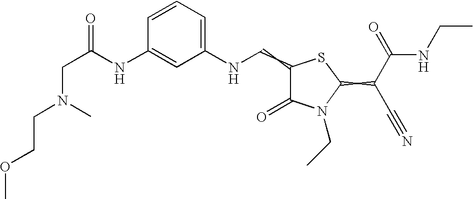 2-cyano-N-ethyl-2-[3-ethyl-5-[1-(3- {2-[(2-methoxy-ethyl)-methyl- amino]-acetylamino}-phenyl- amino)-meth-(E/Z)-ylidene]-4-oxo- thiazolidin-(2-(E or Z))-ylidene]- acetamide | (DMSO-d6, the chief isomer, stored over K₂CO₃): δ = 1.07 (t, 3H); 1.24 (t, 3H); 1.45-1.50 (m, 2H); 2.36 (s, 3H); 2.64 (t, 2H); 3.17-3.23 (m, 4H); 3.27 (s, 3H); 3.46 (t, 1H); 4.21 (q, 2H); 6.96-6.98 (m, 1H); 7.20-7.28 (m, 2H); 7.68-7.71 (m, 2H); 8.00 (d, 1H); 9.74 (s, 1H); 10.36-10.39 (s, 1H) ppm. | 486.60/487 | INTA30/1 |
| 289 | 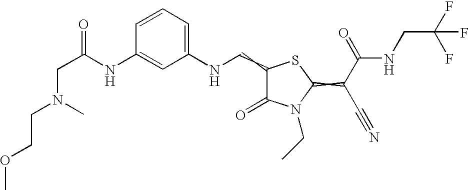 2-cyano-2-[3-ethyl-5-[1-(3-{2-[(2- methoxy-ethyl)-methyl-amino]- acetylamino}-phenylamino)-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 1.25 (t, 3H); 1.45-1.50 (m, 2H); 2.35 (s, 3H); 2.64 (t, 2H); 3.18 (s, 2H); 3.27 (s, 3H); 3.46 (t, 2H); 3.90-3.99 (m, 2H); 4.23 (q, 2H); 6.97-6.99 (m, 1H); 7.21-7.30 (m, 2H); 7.73 (s, 1H); 8.05-8.07 (1H); 8.22 (t, 1H); 9.75 (s, 1H); 10.49-10.51 (s, 1H) ppm. | 540.57/541 | INTA30/1 |
| 290 | 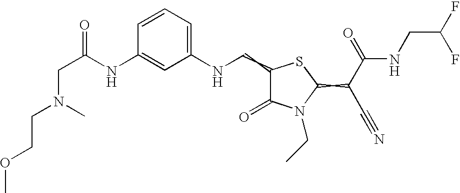 2-cyano-2-[3-ethyl-5-[1-(3-{2-[(2- methoxy-ethyl)-methyl-amino]- acetylamino}-phenylamino)-meth- (E/Z)-ylidene]-4-oxo-thiazolidin-(2- (E or Z))-ylidene]-N-(2,2-difluoro- ethyl)-acetamide | (DMSO-d6, the chief isomer, stored over K₂CO₃): δ = 1.25 (t, 3H); 2.35 (s, 3H); 2.64 (t, 2H); 3.18 (s, 2H); 3.27 (s, 3H); 3.46 (t, 2H); 3.53-3.63 (tt, 2H); 4.30 (q, 2H); 5.90-6.20 (tt, 1H); 6.97-6.99 (m, 1H); 7.21-7.30 (m, 2H); 7.73 (s, 1H); 7.96 (t, 1H); 8.03-8.05 (1H); 9.75 (s, 1H); 10.46-10.49 (1H) ppm. | 506.58/507 | INTA30/1 |
| 291 | 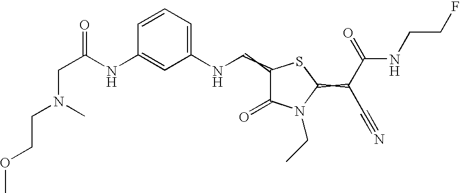 2-cyano-2-[3-ethyl-5-[1-(3-{2-[(2- methoxy-ethyl)-methyl-amino]- acetylamino}-phenylamino)-meth- (E/Z)-ylidene]-4-oxo-thiazolidin-(2- (E or Z))-ylidene]-N-(2-fluoro-ethyl)- acetamide | (DMSO-d6, the chief isomer, stored over K₂CO₃): δ = 1.23 (t, 3H); 2.34 (s, 3H); 2.62 (t, 2H); 3.16 (s, 2H); 3.25 (s, 3H); 3.43-3.52 (m, 2H), 4.21 (q, 2H); 4.41 (t, 1H); 4.53 (t 1H); 6.94-6.96 (m, 1H); 7.19-7.28 (m, 2H); 7.70 (s, 1H); 7.78 (t, 1H); 8.01 (1H); 9.73 (s, 1H); 10.42 (1H) ppm. | 488.59/489 | INTA30/1 |
| 292 | 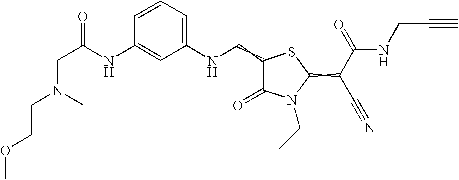 2-cyano-2-[3-ethyl-5-[1-(3-{2-[(2- methoxy-ethyl)-methyl-amino]- acetylamino}-phenylamino)-meth- (E/Z)-ylidene]-4-oxo-thiazolidin-(2- (E or Z))-ylidene]-N-prop-2-ynyl- acetamide | (DMSO-d6, the chief isomer, stored over K₂CO₃): δ = 1.24 (t, 3H); 2.36 (s, 3H); 2.64 (t, 2H); 3.06 (m, 1H); 3.18 (s, 2H); 3.27 (s, 3H); 3.46 (t, 2H); 3.91-3.93 (m, 2H); 4.22 (q, 2H); 6.96-6.98 (m, 1H); 7.21-7.30 (m, 2H); 7.72 (s, 1H); 8.02-8.05 (1H); 8.10 (t, 1H); 9.74 (s, 1H); 10.43-10.46 (1H) ppm. | 496.59/497 | INTA30/1 |
| 293 | 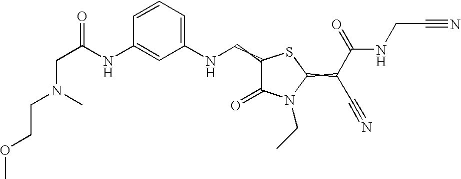 2-cyano-N-cyanomethyl-2-[3-ethyl- 5-[1-(3-{2-[(2-methoxy-ethyl)- methyl-amino]-acetylamino}- phenylamino)-meth-(E/Z)-ylidene]- 4-oxo-thiazolidin-(2-(E or Z))- ylidene]-acetamide | (DMSO-d6, the chief isomer, stored over K₂CO₃): δ = 1.25 (t, 3H); 2.36 (s, 3H); 2.64 (t, 2H); 3.18 (s, 2H); 3.27 (s, 3H); 4.15 (d, 2H); 4.22 (q, 2H); 6.98-7.00 (m, 1H); 7.21-7.31 (m, 2H); 7.74 (s, 1H); 8.05-8.08 (1H); 8.34 (t, 1H); 9.75 (s, 1H); 10.51-10.54 (1H) ppm. | 497.58/498 | INTA30/1 |
| 294 | 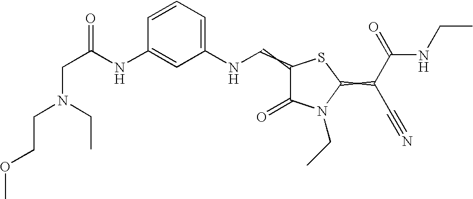 2-cyano-N-ethyl-2-[3-ethyl-5-[1-(3- {2-[ethyl-(2-methoxy-ethyl)-amino]- acetylamino}-phenylamino)-meth- (E/Z)-ylidene]-4-oxo-thiazolidin-(2- (E or Z))-ylidene]-acetamide | (DMSO-d6, the chief isomer, stored over K₂CO₃): δ = 1.01 (t, 3H); 1.07 (t, 3H); 1.24 (t, 3H); 2.66 (q, 2H); 2.72 (t, 2H); 3.17-3.23 (m, 5H); 3.26 (s, 2H); 3.44 (t, 2H); 4.21 (q, 2H); 6.96-6.98 (m, 1H); 7.20-7.31 (m, 2H); 7.70 (m, 2H); 7.99-8.03 (1H); 9.77 (s, 1H); 10.37-10.40 (1H) ppm. | 500.62/501 | INTA31/1 |
| 295 | 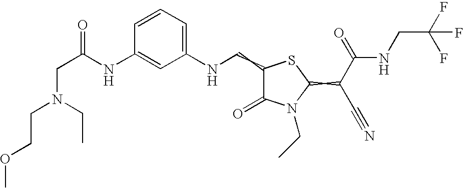 2-cyano-2-[3-ethyl-5-[1-(3-{2- [ethyl-(2-methoxy-ethyl)-amino]- acetylamino}-phenylamino)-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 1.01 (t, 3H); 1.25 (t, 3H); 2.66 (q, 2H); 2.72 (t, 2H); 3.21 (s, 2H); 3.26 (s, 3H); 3.43 (t, 2H); 3.90-3.93 (m, 2H); 4.23 (q, 2H); 6.97-6.99 (m, 1H); 7.20-7.31 (m, 2H); 7.70 (m, 1H); 8.07 (1H); 8.19 (1H); 9.77 (s, 1H); 10.50 (1H) ppm. | 554.60/555 | INTA31/1 |
| 296 | 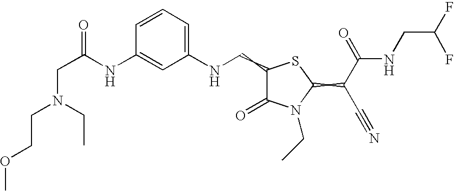 2-cyano-2-[3-ethyl-5-[1-(3-{2- [ethyl-(2-methoxy-ethyl)-amino]- acetylamino}-phenylamino)-meth- (E/Z)-ylidene]-4-oxo-thiazolidin-(2- (E or Z))-ylidene]-N-(2,2-difluoro- ethyl)-acetamide | (DMSO-d6, the chief isomer, stored over K₂CO₃): δ = 1.01 (t, 3H); 1.24 (t, 3H); 2.66 (q, 2H); 2.72 (t, 2H); 3.21 (s, 2H); 3.26 (s, 3H); 3.44 (t, 2H); 3.53-3.63 (tt, 2H); 4.22 (q, 2H); 5.90-6.20 (tt, 1H); 6.97-6.99 (m, 1H); 7.20-7.31 (m, 2H); 7.70 (m, 1H); 7.94 (m, 1H); 8.05 (1H); 9.77 (s, 1H); 10.48 (1H) ppm. | 536.61/537 | INTA31/1 |
| 297 | 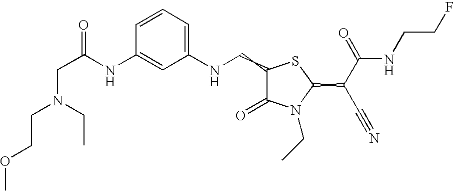 2-cyano-2-[3-ethyl-5-[1-(3-{2- [ethyl-(2-methoxy-ethyl)-amino]- acetylamino}-phenylamino)-meth- (E/Z)-ylidene]-4-oxo-thiazolidin-(2- (E or Z))-ylidene]-N-(2-fluoro-ethyl)- acetamide | (DMSO-d6, the chief isomer, stored over K₂CO₃): δ = 1.01 (t, 3H); 1.24 (t, 3H); 2.66 (q, 2H); 2.72 (t, 2H); 3.21 (s, 2H); 3.26 (s, 3H); 3.42-3.54 (m, 4H); 4.22 (q, 2H); 4.26 (t, 1H); 4.54 (t, 1H); 6.97-6.99 (m, 1H); 7.20-7.31 (m, 2H); 7.70 (m, 1H); 7.80 (t, 1H); 8.01-8.05 (1H); 9.77 (s, 1H); 10.40-10.44 (1H) ppm. | 518.61/519 | INTA31/1 |
| 298 | 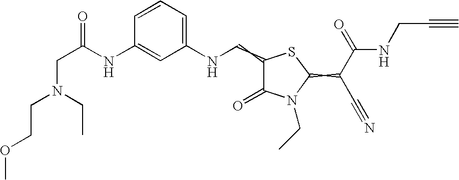 2-cyano-2-[3-ethyl-5-[1-(3-{2- [ethyl-(2-methoxy-ethyl)-amino]- acetylamino}-phenylamino)-meth- (E/Z)-ylidene]-4-oxo-thiazolidin-(2- (E or Z))-ylidene]-N- prop-2-ynyl-acetamide | (DMSO-d6, the chief isomer, stored over K₂CO₃): δ = 1.01 (t, 3H); 1.24 (t, 3H); 2.66 (q, 2H); 2.72 (t, 2H); 3.06 (m, 1H); 3.21 (s, 2H); 3.26 (s, 3H); 3.44 (t, 3H); 3.91-3.93 (m, 2H); 4.22 (q, 2H); 6.97-6.99 (m, 1H); 7.20-7.31 (m, 2H); 7.71 (m, 1H); 8.02-8.06 (1H); 8.09 (t, 1H); 9.78 (s, 1H); 10.43-10.46 (1H) ppm. | 510.62/511 | INTA31/1 |
| 299 | 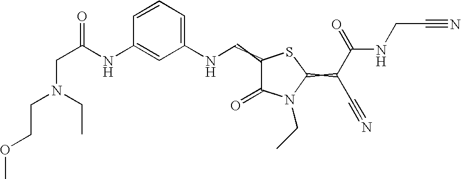 2-cyano-N-cyanomethyl-2-[3-ethyl- 5-[1-(3-{2-[ethyl-(2-methoxy-ethyl)- amino]-acetylamino}-phenyl- amino)-meth-(E/Z)-ylidene]-4-oxo- thiazolidin-(2-(E or Z))-ylidene]- acetamide | (DMSO-d6, the chief isomer, stored over K₂CO₃): δ = 1.20 (t, 3H); 1.25 (t, 3H); 2.66 (q, 2H); 2.72 (t, 2H); 3.21 (s, 2H); 3.26 (s, 3H); 3.44 (t, 3H); 4.15 (d, 2H); 4.22 (q, 2H); 6.98-7.00 (m, 1H); 7.21-7.32 (m, 2H); 7.71 (m, 1H); 8.08 (1H); 8.33 (1H); 9.78 (s, 1H); 10.53 (1H) ppm. | 511.61/512 | INTA31/1 |
| 300 | 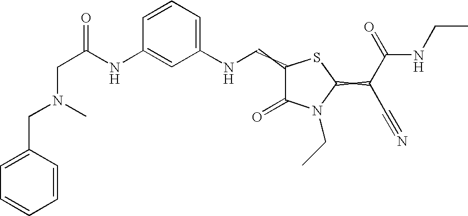 2-[5-[1-{3-[2-(benzyl-methyl-amino)- acetylamino]-phenylamino}-meth- (E/Z)-ylidene]-3-ethyl-4-oxo- thiazolidin-(2-(E or Z))-ylidene]-2- cyano-N-ethyl-acetamide | (DMSO-d6, the chief isomer, stored over K₂CO₃): δ = 1.07 (t, 3H); 1.24 (t, 3H); 2.27 (s, 3H); 3.13-3.23 (m, 4H); 3.65 (s, 2H); 4.22 (q, 4H); 6.98-7.00 (m, 1H); 7.22-7.40 (m, 7H); 7.69 (t, 1H); 7.74 (1H); 8.02 (1H); 9.80 (s, 1H); 10.37-10.39 (1H) ppm. | 518.64/519 | INTA32/1 |
| 301 | 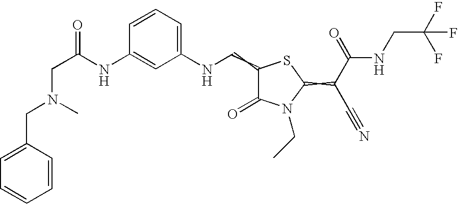 2-[5-[1-{3-[2-(benzyl-methyl-amino)- acetylamino]-phenylamino}-meth- (E/Z)-ylidene]-3-ethyl-4-oxo- thiazolidin-(2-(E or Z))-ylidene]-2- cyano-N-(2,2,2-trifluoro-ethyl)- acetamide | (DMSO-d6, the chief isomer, stored over K₂CO₃): δ = 1.25 (t, 3H); 2.26 (s, 3H); 3.18 (s, 4H); 3.65 (s, 2H); 3.90-3.99 (m, 2H); 4.23 (q, 2H) 6.96-6.98 (m, 1H); 7.23-7.40 (m, 7H); 7.75 (1H); 8.08 (1H); 8.20 (1H); 9.80 (s, 1H); 10.51 (1H) ppm. | 572.61/573 | INTA32/1 |
| 302 | 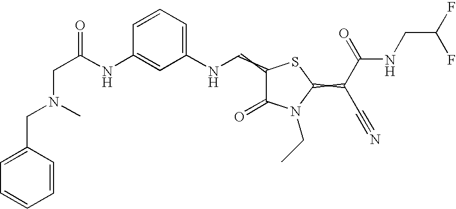 2-[5-[1-{3-[2-(benzyl-methyl-amino)- acetylamino]-phenylamino}-meth- (E/Z)-ylidene]-3-ethyl-4-oxo- thiazolidin-(2-(E or Z))-ylidene]-2- cyano-N-(2,2-difluoro-ethyl)- acetamide | (DMSO-d6, the chief isomer, stored over K₂CO₃): δ = 1.25 (t, 3H); 2.27 (s, 3H); 3.18 (s, 4H); 3.53-3.65 (m, 4H); 3.90-3.99 (m, 2H); 4.23 (q, 2H) 5.90-6.20 (tt, 1H); 6.96-6.98 (m, 1H); 7.23-7.40 (m, 7H); 7.75 (1H); 7.95 (t, 1H); 8.04-8.06 (1H); 9.80 (s, 1H); 10.46-10.48 (1H) ppm. | 554.62/555 | INTA32/1 |
| 303 | 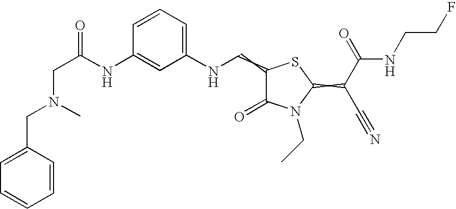 2-[5-[1-{3-[2-(Benzyl-methyl-amino)- acetylamino]-phenylamino}-meth- (E/Z)-ylidene]-3-ethyl-4-oxo- thiazolidin-(2-(E or Z))-ylidene]-2- cyano-N-(2-fluoro-ethyl)-acetamide | (DMSO-d6, the chief isomer, stored over K₂CO₃): δ = 1.25 (t, 3H); 2.27 (s, 3H); 3.18 (s, 4H); 3.44-3.54 (m, 2H); 3.65 (s, 2H); 4.23 (q, 2H); 4.42 (t, 1H); 4.54 (t, 1H); 6.96-6.98 (m, 1H); 7.23-7.40 (m, 7H); 7.74 (1H); 7.79 (t, 1H); 8.02-8.04 (1H); 9.79 (s, 1H); 10.41-10.43 (1H) ppm. | 536.63/537 | INTA32/1 |
| 304 | 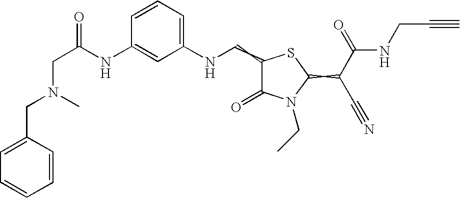 2-[5-[1-{3-[2-(Benzyl-methyl-amino)- acetylamino]-phenylamino}-meth- (E/Z)-ylidene]-3-ethyl-4-oxo- thiazolidin-(2-(E or Z))-ylidene]-2- cyano-N-prop-2-ynyl-acetamide | (DMSO-d6, the chief isomer, stored over K₂CO₃): δ = 1.25 (t, 3H); 2.27 (s, 3H); 3.06 (m, 1H); 3.18 (s, 2H); 3.65 (s, 2H); 3.91-3.93 (m, 2H); 4.22 (q, 4H); 6.96-6.98 (m, 1H); 7.23-7.40 (m, 7H); 7.75 (1H); 8.03-8.10 (m, 2H); 9.79 (s, 1H); 10.43-10.45 (1H) ppm. | 528.64/529 | INTA32/1 |
| 305 | 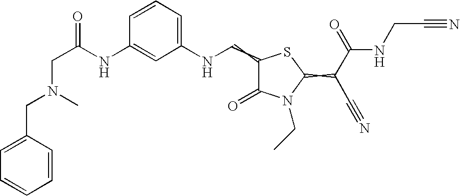 2-[5-[1-{3-[2-(benzyl-methyl-amino)- acetylamino]-phenylamino}-meth- (E/Z)-ylidene]-3-ethyl-4-oxo- thiazolidin-(2-(E or Z))-ylidene]-2- cyano-N-cyanomethyl-acetamide | (DMSO-d6, the chief isomer, stored over K₂CO₃): δ = 1.25 (t, 3H); 2.27 (s, 3H); 3.19 (s, 2H); 3.65 (s, 2H); 4.15 (d, 2H); 4.23 (q, 4H); 6.97-6.99 (m, 1H); 7.24-7.40 (m, 7H); 7.76 (1H); 8.08 (1H); 8.33 (t, 1H); 9.80 (s, 1H); 10.52 (1H) ppm. | 529.63/530 | INTA32/1 |
| 306 | 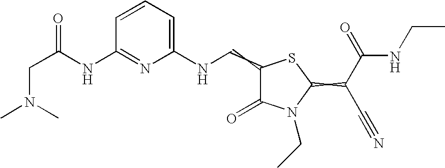 2-cyano-2-[5-[1-[6-(2-dimethyl- amino-acetylamino)-pyridin-2- ylamino]-meth-(E/Z)-ylidene]-3- ethyl-4-oxo-thiazolidin-(2-(E or Z))- ylidene]-N-ethyl-acetamide | (DMSO-d6, the chief isomer, stored over K₂CO₃): δ = 1.07 (t, 3H); 1.24 (t, 3H); 2.30 (s, 6H); 3.13 (s, 2H); 3.16-3.23 (m, 2H); 4.21 (q, 2H); 6.77-6.79 (m, 1H); 7.69-7.76 (m, 3H); 8.59 (1H); 9.88 (s, 1H); 10.72 (1H) ppm. | 443.53/444 | 7/8 |
| 307 | 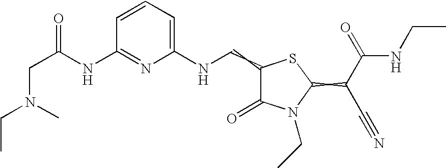 2-cyano-N-ethyl-2-[3-ethyl-5-[1-{6- [2-(ethyl-methyl-amino)-acetyl- amino]-pyridin-2-ylamino}-meth- (E/Z)-ylidene]-4-oxo-thiazolidin-(2- (E or Z))-ylidene]-acetamide | (DMSO-d6, the chief isomer, stored over K₂CO₃): δ = 1.02-1.08 (2t, 6H); 1.24 (t, 3H); 2.30 (s, 3H); 2.53 (q, 2H); 3.18-3.28 (m, 4H); 4.21 (q, 2H); 6.77-6.79 (m, 1H); 7.69-7.76 (m, 3H); 8.58 (1H); 9.85 (s, 1H); 10.73 (1H) ppm. | 457.56/458 | 7/8 |
| 308 | 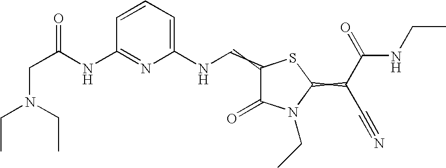 2-cyano-2-[5-[1-[6-(2-diethyl- amino-acetylamino)-pyridin-2- ylamino]-meth-( E/Z)-ylidene]-3- ethyl-4-oxo-thiazolidin-(2-(E or Z))- ylidene]-N-ethyl-acetamide | (DMSO-d6, the chief isomer, stored over K₂CO₃): δ = 1.02 (t, 6H); 1.07 (t, 3H); 1.24 (t, 3H); 2.63 (q, 4H); 3.17-3.23 (m, 4H); 4.21 (q, 2H); 6.78-6.80 (m, 1H); 7.69-7.78 (m, 3H); 8.54-8.56 (1H); 9.84 (s, 1H); 10.72-10.74 (1H) ppm. | 471.59/472 | 7/8 |
| 309 | 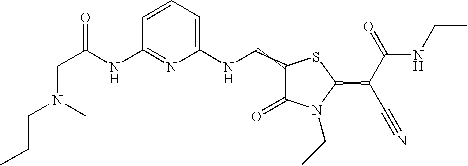 2-cyano-N-ethyl-2-[3-ethyl-5-[1-{6- [2-(methyl-propyl-amino)-acetyl- amino]-pyridin-2-ylamino}-meth- (E/Z)-ylidene]-4-oxo-thiazolidin-(2- (E or Z))-ylidene]-acetamide | (DMSO-d6, the chief isomer, stored over K₂CO₃): δ = 0.91 (t, 3H); 1.07 (t, 3H); 1.24 (t, 3H); 1.47 (q, 2H); 2.31 (s, 3H); 2.42 (t, 2H); 3.18-3.28 (m, 4H); 4.21 (q, 2H); 6.77-6.79 (m, 1H); 7.69-7.77 (m, 3H); 8.57 (1H); 9.85 (s, 1H); 10.71 (1H) ppm. | 471.59/472 | 7/8 |
| 310 | 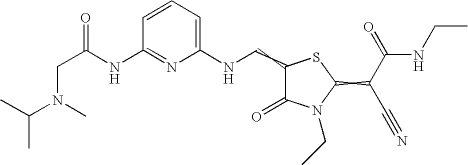 2-cyano-N-ethyl-2-[3-ethyl-5-[1-{6- [2-(isopropyl-methyl-amino)-acetyl- amino]-pyridin-2-ylamino}-meth- (E/Z)-ylidene]-4-oxo-thiazolidin-(2- (E or Z))-ylidene]-acetamide | (DMSO-d6, the chief isomer, stored over K₂CO₃): δ = 1.02 (d, 6H); 1.07 (t, 3H); 1.24 (t, 3H); 2.27 (s, 3H); 2.87-2.94 (m, 1H); 3.15-3.23 (m, 4H); 4.21 (q, 2H); 6.78-6.80 (m, 1H); 7.70-7.76 (m, 3H); 8.55 (1H); 9.79 (s, 1H); 10.73 (1H) ppm. | 471.59/472 | 7/8 |
| 311 | 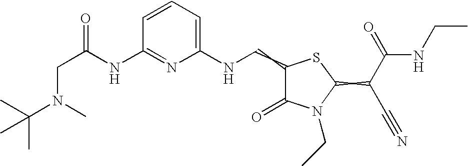 2-[5-[1-{6-[2-(tert-butyl-methyl- amino)-acetylamino]-pyridin-2-yl- amino}-meth-(E/Z)-ylidene]-3-ethyl- 4-oxo-thiazolidin-(2-(E or Z))- ylidene]-2-cyano-N-ethyl- acetamide | (DMSO-d6, the chief isomer, stored over K₂CO₃): δ = 1.07 (t, 3H); 1.09 (s, 9H); 1.24 (t, 3H); 2.28 (s, 3H); 3.17-3.23 (m, 4H); 4.21 (q, 2H); 6.78-6.80 (m, 1H); 7.70-7.73 (m, 3H); 8.54 (1H); 9.82 (s, 1H); 10.74 (1H) ppm. | 485.61/486 | 7/8 |
| 312 | 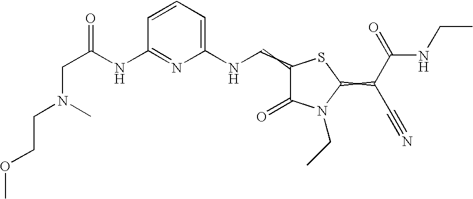 2-cyano-N-ethyl-2-[3-ethyl-5-[1-(6- {2-[(2-methoxy-ethyl)-methyl- amino]-acetylamino}-pyridin-2- ylamino)-meth-(E/Z)-ylidene]-4- oxo-thiazolidin-(2-(E or Z))- ylidene]-acetamide | (DMSO-d6, the chief isomer, stored over K₂CO₃): δ = 1.07 (t, 3H); 1.24 (t, 3H); 2.37 (s, 3H); 2.66 (t, 2H); 3.17-3.23 (m, 4H); 3.27 (s, 3H); 3.44 (t, 2H); 4.21 (q, 2H); 6.77-6.79 (m, 1H); 7.69-7.78 (m, 3H); 8.60 (1H), 9.94 (s, 1H); 10.72 (1H) ppm. | 487.58/488 | 7/8 |
| 313 | 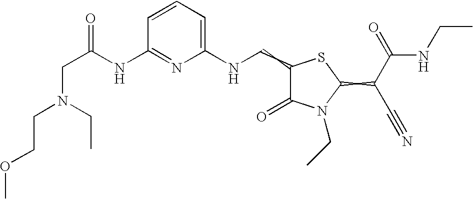 2-cyano-N-ethyl-2-[3-ethyl-5-[1-(6- {2-[ethyl-(2-methoxy-ethyl)-amino]- acetylamino}-pyridin-2-ylamino)- meth-(E/Z)-ylidene]-4-oxo- thiazolidin-(2-(E or Z))-ylidene]- acetamide | (DMSO-d6, the chief isomer, stored over K₂CO₃): δ = 1.00 (t, 3H); 1.07 (t, 3H); 1.24 (t, 3H); 2.69-2.75 (m, 4H); 3.17-3.23 (m, 4H); 3.29 (s, 3H); 3.42 (t, 2H); 4.21 (q, 2H); 6.77-6.79 (m, 1H); 7.69-7.78 (m, 3H); 8.58-8.61 (1H); 9.97 (s, 1H); 10.70-10.73 (1H) ppm. | 501.61/502 | 7/8 |
| 314 | 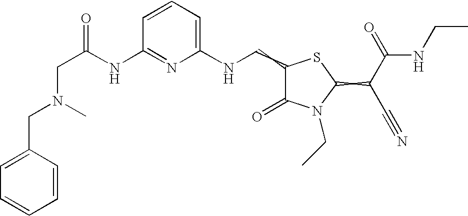 2-[5-[1-{6-[2-(benzyl-methyl-amino)- acetylamino]-pyridin-2-ylamino}- meth-(E/Z)-ylidene]-3-ethyl-4-oxo- thiazolidin-(2-(E or Z))-ylidene]-2- cyano-N-ethyl-acetamide | (DMSO-d6, the chief isomer, stored over K₂CO₃): δ = 1.07 (t, 3H); 1.26 (t, 3H); 2.27 (s, 3H); 3.17-3.24 (m, 2H); 3.28 (s, 2H); 3.65 (s, 2H); 4.23 (q, 2H); 6.78-6.80 (m, 1H); 7.23-7.28 (m, 1H); 7.36-7.38 (m, 5H); 7.69-7.77 (m, 3H); 8.64 (1H); 10.04 (1H) ppm. | 519.63/520 | 7/8 |
| 315 | 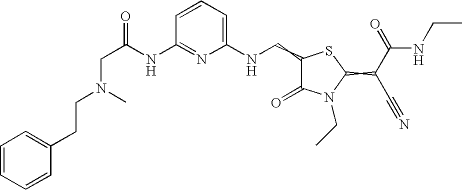 2-cyano-N-ethyl-2-[3-ethyl-5-[1-{6- [2-(methyl-phenethyl-amino)- acetylamino]-pyridin-2-ylamino}- meth-(E/Z)-ylidene]-4-oxo- thiazolidin-(2-(E or Z))-ylidene]- acetamide | (DMSO-d6, the chief isomer, stored over K₂CO₃): δ = 1.07 (t, 3H); 1.26 (t, 3H); 2.37 (s, 3H); 2.73-2.79 (m, 4H); 3.17-3.25 (m, 4H); 4.23 (q, 2H); 6.77-6.79 (m, 1H); 7.12 (t, 1H); 7.22-7.28 (m, 4H); 7.67-7.77 (m, 3H); 8.56 (s, 1H); 9.60 (s, 1H); 10.65 (1H) ppm. | 533.66/534 | 7/8 |
| 316 | 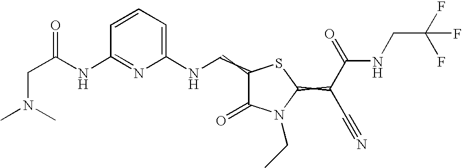 2-cyano-2-[5-[1-[6-(2-dimethyl- amino-acetylamino)-pyridin-2- ylamino]-meth-(E/Z)-ylidene]-3- ethyl-4-oxo-thiazolidin-(2-(E or Z))- ylidene]-N-(2,2,2-trifluoro-ethyl)- acetamide | (DMSO-d6, the chief isomer, stored over K₂CO₃): δ = 1.26 (t, 3H); 2.30 (s, 6H); 3.13 (s, 2H); 3.91-4.00 (m, 2H); 4.23 (q, 2H); 6.77-6.79 (m, 1H); 7.70-7.76 (m, 2H); 8.26-8.29 (1H), 8.64 (1H); 9.91 (s, 1H); 10.84 (1H) ppm. | 497.50/498 | 7/8 |
| 317 | 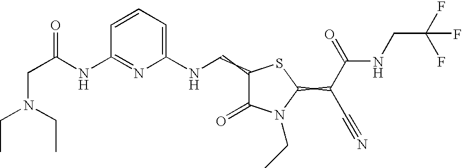 2-cyano-2-[5-[1-[6-(2-diethyl- amino-acetylamino)-pyridin-2- ylamino]-meth-(E/Z)-ylidene]-3- ethyl-4-oxo-thiazolidin-(2-(E or Z))- ylidene]-N-(2,2,2-tri-fluoro-ethyl)- acetamide | (DMSO-d6, the chief isomer, stored over K₂CO₃): δ = 1.02 (t, 6H); 1.26 (t, 3H); 2.62 (q, 4H); 3.22 (s, 2H); 3.91-3.99 (m, 2H); 4.23 (q, 2H); 6.78-6.80 (m, 1H); 7.71-7.76 (m, 2H); 8.27 (t, 1H); 8.60 (1H); 9.86 (s, 1H); 10.84 (1H) ppm. | 525.56/526 | 7/8 |
| 318 | 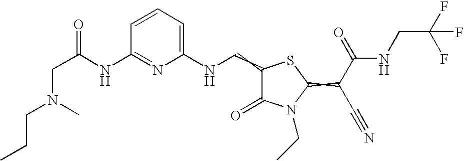 2-cyano-2-[3-ethyl-5-[1-{6-[2- (methyl-propyl-amino)-acetyl- amino]-pyridin-2-ylamino}-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 0.91 (t, 3H); 1.26 (t, 3H); 1.47 (q, 2H); 2.31 (s, 3H); 2.43 (t, 2H); 3.18 (s, 2H); 3.91-3.99 (m, 2H); 4.23 (q, 2H); 6.77-6.79 (m, 1H); 7.70-7.77 (m, 2H); 8.26 (t, 1H); 8.61-8.63 (1H); 9.88 (s, 1H); 10.81-10.83 (1H) ppm. | 525.56/526 | 7/8 |
| 319 | 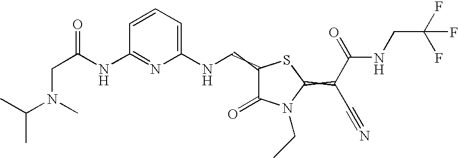 2-cyano-2-[3-ethyl-5-[1-{6-[2-(iso- propyl-methyl-amino)-acetyl- amino]-pyridin-2-ylamino}-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 1.02 (d, 6H); 1.26 (t, 3H); 2.27 (s, 3H); 2.88-2.94 (m, 1H); 3.16 (s, 2H); 3.91-3.99 (m, 2H); 4.23 (q, 2H); 6.78-6.80 (m, 1H); 7.70-7.78 (m, 2H); 8.27 (t, 1H); 8.60 (1H); 9.81 (s, 1H); 10.84 (1H) ppm. | 525.56/526 | 7/8 |
| 320 | 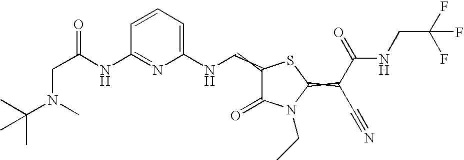 2-[5-[1-{6-[2-(tert-butyl-methyl- amino)-acetylamino]-pyridin-2-yl- amino}-meth-(E/Z)-ylidene]-3-ethyl- 4-oxo-thiazolidin-(2-(E or Z))- ylidene]-2-cyano-N-(2,2,2-trifluoro- ethyl)-acetamide | (DMSO-d6, the chief isomer, stored over K₂CO₃): δ = 1.09 (s, 9H); 1.26 (t, 3H); 2.29 (s, 3H); 3.18 (s, 2H); 3.91-3.99 (m, 2H); 4.23 (q, 2H); 6.79-6.81 (m, 1H); 7.71-7.79 (m, 2H); 8.26 (t, 1H); 8.58-8.60 (1H); 9.84 (s, 1H); 10.82-10.84 (1H) ppm. | 539.58/540 | 7/8 |
| 321 | 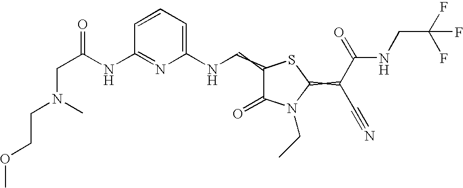 2-cyano-2-[3-ethyl-5-[1-(6-{2-[(2- methoxy-ethyl)-methyl-amino]- acetylamino}-pyridin-2-ylamino)- meth-(E/Z)-ylidene]-4-oxo- thiazolidin-(2-(E or Z))-ylidene]-N- (2,2,2-trifluoro-ethyl)-acetamide | (DMSO-d6, the chief isomer, stored over K₂CO₃): δ = 1.25 (t, 3H); 2.38 (s, 3H); 2.66 (t, 2H); 3.23 (s, 2H); 3.29 (s, 3H); 3.44 (t, 2H); 3.91-3.99 (m, 2H); 4.23 (q, 2H); 6.77-6.79 (m, 1H); 7.70-7.79 (m, 3H); 8.27 (t, 1H); 8.63-8.66 (1H); 9.96 (s, 1H); 10.81-10.84 (1H) ppm. | 541.56/542 | 7/8 |
| 322 | 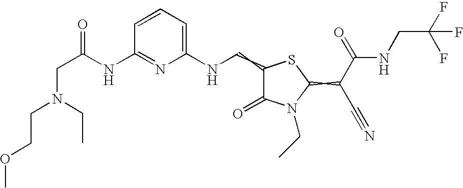 2-cyano-2-[3-ethyl-5-[1-(6-{2- [ethyl-(2-methoxy-ethyl)-amino]- acetyl-amino}-pyridin-2-ylamino)- meth-(E/Z)-ylidene]-4-oxo- thiazolidin-(2-(E or Z))-ylidene]-N- (2,2,2-trifluoro-ethyl)-acetamide | (DMSO-d6, the chief isomer, stored over K₂CO₃): δ = 1.00 (t, 3H); 1.26 (t, 3H); 2.69-2.75 (m, 4H); 3.26 (s, 2H); 3.30 (s, 3H); 3.42 (t, 2H); 3.91-3.99 (m, 2H); 4.23 (q, 2H); 6.77-6.79 (m, 1H); 7.70-7.80 (m, 2H); 8.27 (t, 1H); 8.63-8.66 (1H); 9.99 (s, 1H); 10.81-10.84 (1H) ppm. | 555.58/556 | 7/8 |
| 323 | 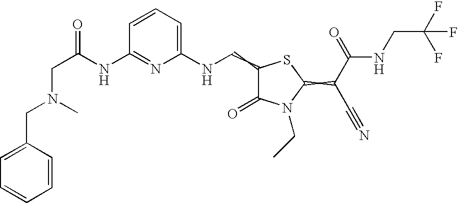 2-[5-[1-{6-[2-(benzyl-methyl- amino)-acetylamino]-pyridin-2-yl- amino}-meth-(E/Z)-ylidene]-3-ethyl- 4-oxo-thiazolidin-(2-(E or Z))- ylidene]-2-cyano-N-(2,2,2-tri-fluoro- ethyl)-acetamide | (DMSO-d6, the chief isomer, stored over K₂CO₃): δ = 1.27 (t, 3H); 2.27 (s, 3H); 3.28 (s, 2H); 3.65 (s, 2H); 3.91-4.00 (m, 2H); 4.25 (q, 2H); 6.78-6.80 (m, 1H); 7.23-7.29 (m, 1H); 7.36-7.38 (m, 4H); 7.70-7.76 (m, 2H); 8.27 (t, 1H); 8.67-8.70 (1H); 10.05 (1H); 10.81-10.84 (1H) ppm. | 573.60/574 | 7/8 |
| 324 | 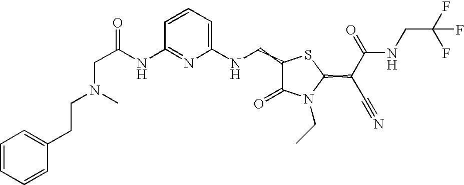 2-cyano-2-[3-ethyl-5-[1-{6-[2- (methyl-phenethyl-amino)-acetyl- amino]-pyridin-2-ylamino}-meth- (E/Z)-ylidene]-4-oxo-thiazolidin-(2- (E or Z))-ylidene]-N-(2,2,2-tri- fluoro-ethyl)-acetamide | (DMSO-d6, the chief isomer, stored over K₂CO₃): δ = 1.27 (t, 3H); 2.37 (s, 3H); 2.74-2.79 (m, 4H); 3.25 (s, 2H); 3.91-4.00 (m, 2H); 4.25 (q, 2H); 6.77-6.79 (m, 1H); 7.12 (t, 1H); 7.22-7.28 (m, 4H); 7.68-7.74 (m, 2H); 8.26 (t, 1H); 8.58-8.62 (1H); 9.61 (s, 1H); 10.78-10.81 (1H) ppm. | 587.63/588 | 7/8 |
| 325 | 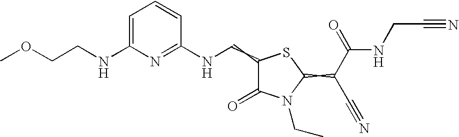 2-cyano-N-cyanomethyl-2-[3-ethyl- 5-[1-[6-(2-methoxy-ethylamino)- pyridin-2-ylamino]-meth-(E/Z)- ylidene]-4-oxo-thiazolidin-(2-(E or Z))-ylidene]-acetamide | | 427.488/428 | INTT10/INT83/5 |
| 326 | 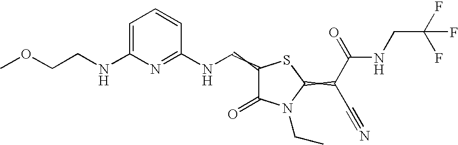 2-cyano-2-[3-ethyl-5-[1-[6-(2- methoxy-ethylamino)-pyridin-2- ylamino]-meth-(E/Z)-ylidene]-4- oxo-thiazolidin-(2-(E or Z))- ylidene]-N-(2,2,2-trifluoro-ethyl)- acetamide | | 470.477/471 | INTT8/INT83/5 |
| 327 | 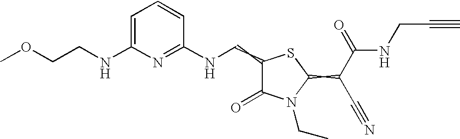 2-cyano-2-[3-ethyl-5-[1-[6-(2- methoxy-ethylamino)-pyridin-2- ylamino]-meth-(E/Z)-ylidene]-4- oxo-thiazolidin-(2-(E or Z))- ylidene]-N-prop-2-ynyl-acetamide | | 426.501/427 | INTT9/INT83/5 |
| 328 | 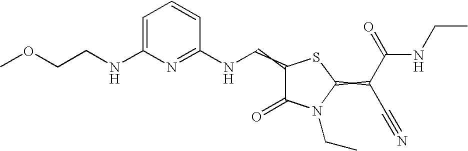 2-cyano-N-ethyl-2-[3-ethyl-5-[1-[6- (2-methoxy-ethylamino)-pyridin-2- ylamino]-meth-(E/Z)-ylidene]-4- oxo-thiazolidin-(2-(E or Z))- ylidene]-acetamide | | 416.506/417 | INTT7/INT83/5 |
| 329 | 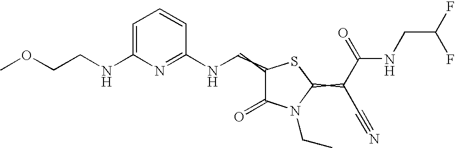 2-cyano-N-(2,2-difluoro-ethyl)-2-[3- ethyl-5-[1-[6-(2-methoxy- ethylamino)-pyridin-2-ylamino]- meth-(E/Z)-ylidene]-4-oxo- thiazolidin-(2-(E or Z))-ylidene]- acetamide | | 452.486/453 | INTT11/INT83/5 |
| 330 | 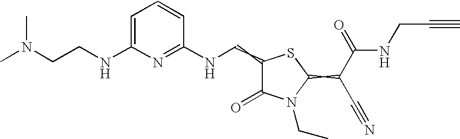 2-cyano-2-[5-[1-[6-(2- dimethylamino-ethylamino)-pyridin- 2-ylamino]-meth-(E/Z)-ylidene]-3- ethyl-4-oxo-thiazolidin-(2-(E or Z))- ylidene]-N-prop-2-ynyl-acetamide | | 439.543/440 | INTT9/INT120/5 |
| 331 | 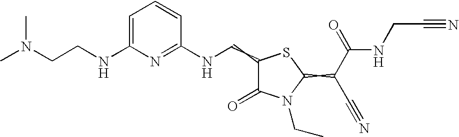 2-cyano-N-cyanomethyl-2-[5-[1-[6- (2-dimethylamino-ethylamino)- pyridin-2-ylamino]-meth-(E/Z)- ylidene]-3-ethyl-4-oxo-thiazolidin- (2-(E or Z))-ylidene]-acetamide | | 440.531/441 | INTT10/INT120/5 |
| 332 | 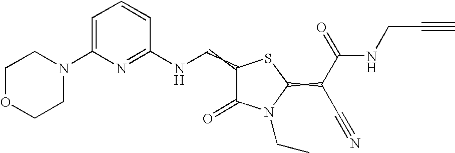 2-cyano-2-[3-ethyl-5-[1-(6- morpholin-4-yl-pyridin-2-ylamino)- meth-(E/Z)-ylidene]-4-oxo- thiazolidin-(2-(E or Z))-ylidene]-N- prop-2-ynyl-acetamide | | 438.512/439 | INTT9/INT116/5 |
| 333 | 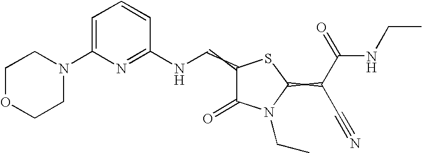 2-cyano-N-ethyl-2-[3-ethyl-5-[1-(6- morpholin-4-yl-pyridin-2-ylamino)- meth-(E/Z)-ylidene]-4-oxo- thiazolidin-(2-(E or Z))-ylidene]- acetamide | | 428.517/429 | INTT7/INT116/5 |
| 334 | 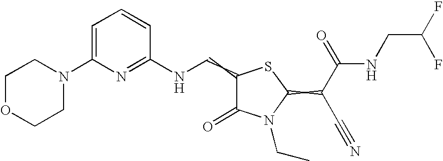 2-cyano-N-(2,2-difluoro-ethyl)-2-[3- ethyl-5-[1-(6-morpholin-4-yl-pyridin- 2-ylamino)-meth-(E/Z)-ylidene]-4- oxo-thiazolidin-(2-(E or Z))- ylidene]-acetamide | | 464.498/465 | INTT11/INT116/5 |
| 335 | 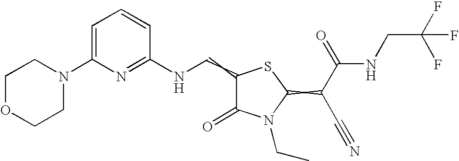 2-cyano-2-[3-ethyl-5-[1-(6- morpholin-4-yl-pyridin-2-ylamino)- meth-(E/Z)-ylidene]-4-oxo- thiazolidin-(2-(E or Z))-ylidene]-N- (2,2,2-trifluoro-ethyl)-acetamide | | 482.488/483 | INTT8/INT116/5 |
| 336 | 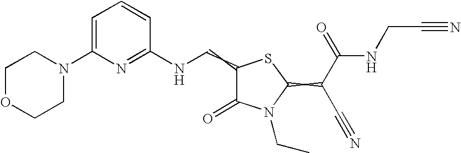 2-cyano-N-cyanomethyl-2-[3-ethyl- 5-[1-(6-morpholin-4-yl-pyridin-2- ylamino)-meth-(E/Z)-ylidene]-4-xo- thiazolidin-(2-(E or Z))-ylidene]- acetamide | | 439.499 440 | INTT10/INT116/5 |
| 337 | 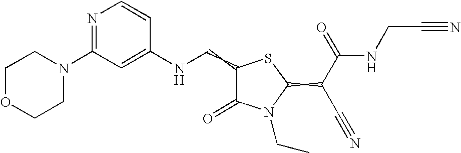 2-cyano-N-cyanomethyl-2-[3-ethyl- 5-[1-(2-morpholin-4-yl-pyridin-4- ylamino)-meth-(E/Z)-ylidene]-4- oxo-thiazolidin-(2-(E or Z))- ylidene]-acetamide | | 439.499/440 | INTE69/198 |
| 338 | 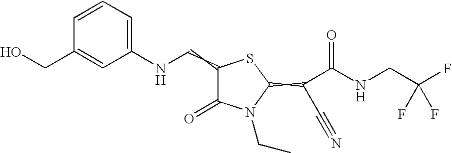 2-cyano-2-[3-ethyl-5-[1-(3- hydroxymethyl-phenylamino)-meth- (E/Z)-ylidene]-4-oxo-thiazolidin-(2- (E or Z))-ylidene]-N-(2,2,2-trifluoro- ethyl)-acetamide | (DMSO-d6, the chief isomer, stored over K₂CO₃): δ = 1.27 (t, 3H); 3.95 (m, 2H); 4.24 (q, 2H); 4.50 (d, 2H); 5.27 (t, 1H); 7.01 (d, 1H); 7.16 (d, 1H); 7.26-7.32 (m, 2H); 8.08-8.24 (m, 2H); 10.47 (s, 1H) ppm. | | INTA33/1 |
| 339 | 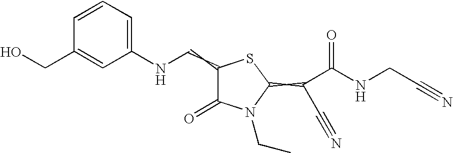 2-cyano-N-cyanomethyl-2-[3-ethyl- 5-[1-(3-hydroxymethyl- phenylamino)-meth-(E/Z)-ylidene]- 4-oxo-thiazolidin-(2-(E or Z))- ylidene]-acetamide | (DMSO-D6, the chief isomer, stored over K2CO3): δ = 1.27 (t, 3H); 4.17 (d, 2H); 4.24 (q, 2H); 4.50 (d, 2H); 5.26 (t, 1H); 7.02 (d, 1H); 7.16 (d, 1H); 7.25-7.33 (m, 2H); 8.16 (s, 1H); 8.32 (s, 1H); 10.49 (s, 1H) ppm. | | INTA33/1 |
| 340 | 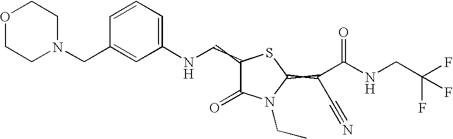 2-cyano-2-[3-ethyl-5-[1-(3- morpholin-4-ylmethyl- phenylamino)-meth-(E/Z)-ylidene]- 4-oxo-thiazolidin-(2-(E or Z))- ylidene]-N-(2,2,2-trifluoro-ethyl)- acetamide | (DMSO-D6, the chief isomer, stored over K2CO3): δ = 1.23 (t, 3H); 2.33 (s, 4H); 3.42 (s, 2H); 3.54 (m, 4H); 3.93 (m, 2H); 4.20 (q, 2H); 6.98 (d, 1H); 7.16 (d, 1H); 7.20-7.28 (m, 2H); 8.10 (s, 1H); 8.17 (s, 1H); 10.40 (s, 1H) ppm. | | 338/12 und 8 |
| 341 | 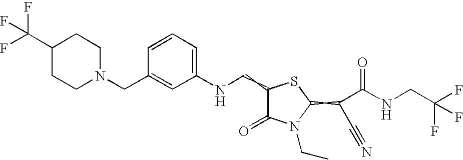 2-cyano-2-[3-ethyl-4-oxo-5-[1-[3-(4- trifluoromethyl-piperidin-1- ylmethyl)-phenylamino]-meth- (E/Z)-ylidene]-thiazolidin-(2-(E or Z))-ylidene]-N-(2,2,2-trifluoro- ethyl)-acetamide | (DMSO-D6, the chief isomer, stored over K2CO3): δ = 1.23 (t, 3H); 1.44 (m, 2H); 1.75 (d, 2H); 1.94 (t, 2H); 2.23 (m, 1H); 2.87 (d, 2H); 3.43 (s, 2H); 3.93 (m, 2H); 4.20 (q, 2H); 6.97 (d, 1H); 7.17 (d, 1H); 7.20-7.27 (m, 2H); 8.09 (d, 1H); 8.19 (t, 1H); 10.40 (d, 1H) ppm. | | 338/12 und 8 |
| 342 | 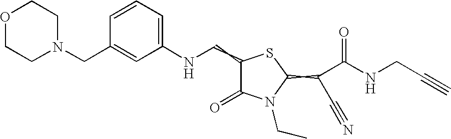 2-cyano-2-[3-ethyl-5-[1-(3- morpholin-4-ylmethyl- phenylamino)-meth-(E/Z)- ylidene]-4-oxo-thiazolidin-(2-(E or Z))-ylidene]-N-prop-2-ynyl- acetamide | (DMSO-D6, the chief isomer, stored over K2CO3): δ = 1.21 (t, 3H); 2.34 (s, 4H); 3.02 (s, 1H); 3.40 (s, 2H); 3.56 (m, 4H); 3.89 (m, 2H); 4.19 (q, 2H); 6.97 (d, 1H); 7.17 (d, 1H); 7.20-7.28 (m, 2H); 8.08 (m, 2H); 10.36 (d, 1H) ppm. | | 4/12 und 8 |
| 343 | 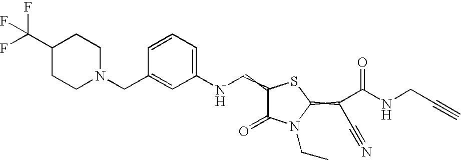 2-cyano-2-[3-ethyl-4-oxo-5-[1-[3-(4- trifluoromethyl-piperidin-1- ylmethyl)-phenylamino]-meth- (E/Z)-ylidene]-thiazolidin-(2-(E or Z))-ylidene]-N-prop-2-ynyl- acetamide | (DMSO-D6, the chief isomer, stored over K2CO3): δ = 1.20 (t, 3H); 1.42 (m, 2H); 1.74 (d, 2H); 1.93 (t, 2H); 2.23 (m, 1H); 2.85 (d, 2H); 3.02 (s, 1H); 3.42 (s, 2H); 3.89 (m, 2H); 4.20 (q, 2H); 6.96 (d, 1H); 7.16 (d, 1H); 7.22-7.27 (m, 2H); 8.07 (m, 2H); 10.34 (d, 1H) ppm. | | 4/12 und 8 |
| 344 | 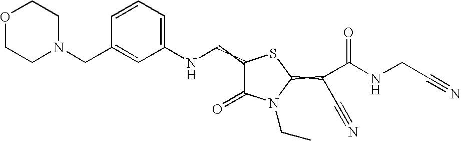 2-cyano-N-cyanomethyl-2-[3-ethyl- 5-[1-(3-morpholin-4-ylmethyl- phenylamino)-meth-(E/Z)-ylidene]- 4-oxo-thiazolidin-(2-(E or Z))- ylidene]-acetamide | (DMSO-D6, the chief isomer, stored over K2CO3): δ = 1.22 (t, 3H); 2.35 (s, 4H); 3.45 (s, 2H); 3.57 (m, 4H); 4.14 (d, 2H); 4.20 (q, 2H); 6.98 (d, 1H); 7.17 (d, 1H); 7.23-7.28 (m, 2H); 8.10 (m, 1H); 8.32 (t, 1H); 10.43 (s, 1H) ppm. | | 339/12 und 8 |
| 345 | 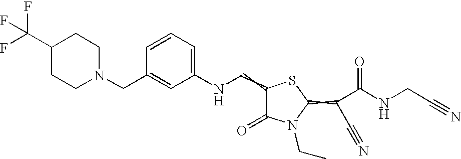 2-cyano-N-cyanomethyl-2-[3-ethyl- 4-oxo-5-[1-[3-(4-trifluoromethyl- piperidin-1-ylmethyl)-phenylamino]- meth-(E/Z)-ylidene]-thiazolidin-(2- (E or Z))-ylidene]-acetamide | (DMSO-D6, the chief isomer, stored over K2CO3): δ = 1.21 (t, 3H); 1.44 (m, 2H); 1.76 (d, 2H); 1.94 (t, 2H); 2.25 (m, 1H); 2.87 (d, 2H); 3.45 (s, 2H); 4.13 (d, 2H); 4.20 (q, 2H); 6.97 (d, 1H); 7.16 (d, 1H); 7.22-7.28 (m, 2H); 8.12 (m, 1H); 8.32 (t, 1H); 10.43 (m, 1H) ppm. | | 339/12 und 8 |
| 346 | 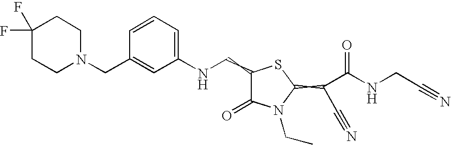 2-cyano-N-cyanomethyl-2-[5-[1-[3- (4,4-difluoro-piperidin-1-ylmethyl)- phenylamino]-meth-(E/Z)-ylidene]-3- ethyl-4-oxo-thiazolidin-(2-(E or Z))- ylidene]-acetamide | (DMSO-D6, the chief isomer, stored over K2CO3): δ = 1.21 (t, 3H); 1.93 (m, 4H); 2.48 (m, 4H); 3.49 (s, 2H); 4.12 (d, 2H); 4.20 (q, 2H); 6.98 (d, 1H); 7.17 (d, 1H); 7.24-7.29 (m, 2H); 8.10 (d, 1H); 8.31 (t, 1H); 10.42 (d, 1H) ppm. | | 339/12 und 8 |
| 347 | 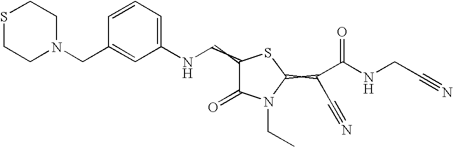 2-cyano-N-cyanomethyl-2-[3-ethyl- 4-oxo-5-[1-(3-thiomorpholin-4- ylmethyl-phenylamino)-meth-(E/Z)- ylidene]-thiazolidin-(2-(E or Z))- ylidene]-acetamide | (DMSO-D6, the chief isomer, stored over K2CO3): δ = 1.21 (t, 3H); 2.58 (m, 8H); 3.46 (s, 2H); 4.12 (d, 2H); 4.20 (q, 2H); 6.96 (d, 1H); 7.17 (d, 1H); 7.21-7.29 (m, 2H); 8.11 (d, 1H); 8.32 (t, 1H); 10.41 (d, 1H) ppm. | | 339/12 und 8 |
| 348 | 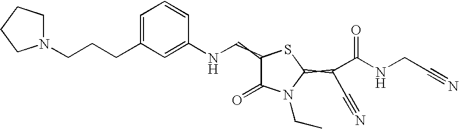 2-cyano-N-cyanomethyl-2-[3-ethyl- 4-oxo-5-[1-[3-(3-pyrrolidin-1-yl-prop- yl)-phenylamino]-meth-(E/Z)- ylidene]-thiazolidin-(2-(E or Z))- ylidene]-acetamide | (DMSO-D6, the chief isomer, stored over K2CO3): δ = 1.20 (t, 3H); 1.64 (m, 4H); 1.72 (m, 2H); 2.42 (m, 6H); 2.57 (t, 2H); 4.11 (d, 2H); 4.20 (q, 2H); 6.89 (d, 1H); 7.06-7.24 (m, 3H); 8.12 (s, 1H); 8.31 (t, 1H); 10.40 (s, 1H) ppm. | | INTA34/1 |
| 349 | 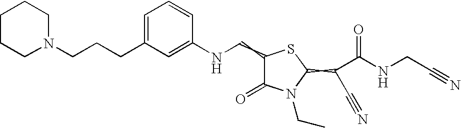 2-cyano-N-cyanomethyl-2-[3-ethyl- 4-oxo-5-[1-[3-(3-piperidin-1-yl- propyl)-phenylamino]-meth-(E/Z)- ylidene]-thiazolidin-(2-(E or Z))- ylidene]-acetamide | (DMSO-D6, the chief isomer, stored over K2CO3): δ = 1.20 (t, 3H); 1.33 (m, 2H); 1.45 (m, 4H); 1.68 (m, 2H); 2.19 (t, 2H); 2.26 (m, 4H); 2.53 (t, 2H); 4.12 (d, 2H); 4.20 (q, 2H); 6.88 (d, 1H); 7.07 (d, 1H); 7.13 (s, 1H); 7.20 (t, 1H); 8.12 (s, 1H); 8.28 (m, 1H); 10.32 (s, 1H) ppm. | | INTA35/1 |
| 350 | 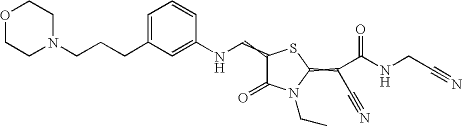 2-cyano-N-cyanomethyl-2-[3-ethyl- 5-[1-[3-(3-morpholin-4-yl-propyl)- phenylamino]-meth-(E/Z)-ylidene]- 4-oxo-thiazolidin-(2-(E or Z))- ylidene]-acetamide | (DMSO-D6, the chief isomer, stored over K2CO3): δ = 1.21 (t, 3H); 1.70 (m, 2H); 2.23 (t, 2H); 2.29 (m, 4H); 2.55 (t, 2H); 3.54 (t, 4H); 4.12 (d, 2H); 4.20 (q, 2H); 6.88 (d, 1H); 7.08 (d, 1H); 7.14 (s, 1H); 7.20 (t, 1H); 8.11 (s, 1H); 8.29 (t, 1H); 10.32 (s, 1H) ppm. | | INTA36/1 |
| 351 | 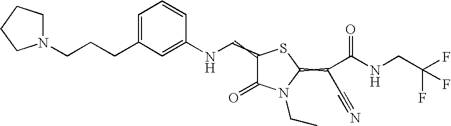 2-cyano-2-[3-ethyl-4-oxo-5-[1-[3-(3- pyrrolidin-1-yl-propyl)-phenylamino]- meth-(E/Z)-ylidene]-thiazolidin- (2-(E or Z))-ylidene]-N-(2,2,2- trifluoro-ethyl)- acetamide | (DMSO-D6, the chief isomer, stored over K2CO3): δ = 1.21 (t, 3H); 1.63 (m, 4H); 1.69 (m, 2H); 2.32-2.40 (m, 6H); 2.56 (t, 2H); 3.92 (m, 2H); 4.21 (q, 2H); 6.88 (d, 1H); 7.08 (d, 1H); 7.13 (s, 1H); 7.20 (t, 1H); 8.12 (s, 1H); 8.15 (t, 1H); 10.38 (s, 1H) ppm. | | INTA34/1 |
| 352 | 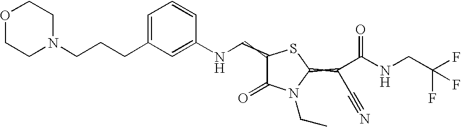 2-cyano-2-[3-ethyl-5-[1-[3-(3- morpholin-4-yl-propyl)- phenylamino]-meth-(E/Z)-ylidene]- 4-oxo-thiazolidin-(2-(E or Z))- ylidene]-N-(2,2,2-trifluoro-ethyl)- acetamide | (DMSO-D6, the chief isomer, stored over K2CO3): δ = 1.20 (t, 3H); 1.68 (m, 2H); 2.27 (t, 2H); 2.32 (m, 4H); 2.55 (t, 2H); 3.53 (t, 4H); 3.92 (m, 2H); 4.20 (q, 2H); 6.88 (d, 1H); 7.05-7.23 (m, 3H); 8.11 (s, 1H); 8.17 (m, 1H); 10.32 (m, 1H) ppm. | | INTA36/1 |
| 353 | 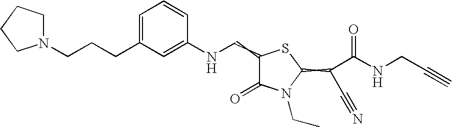 2-cyano-2-[3-ethyl-4-oxo-5-[1-[3-(3- pyrrolidin-1-yl-propyl)-phenylamino]- meth-(E/Z)-ylidene]-thiazolidin- (2Z)-ylidene]-N-prop-2-ynyl- acetamide | (DMSO-D6, the chief isomer, stored over K2CO3): δ = 1.20 (t, 3H); 1.62 (m, 4H); 1.68 (m, 2H); 2.32-2.40 (m, 6H); 2.54 (t, 2H); 3.01 (s, 1H); 3.88 (m, 2H); 4.19 (q, 2H); 6.87 (d, 1H); 7.06 (d, 1H); 7.12 (s, 1H); 7.20 (t, 1H); 8.04-8.13 (m, 2H); 10.28 (s, 1H) ppm. | | INTA34/1 |
| 354 | 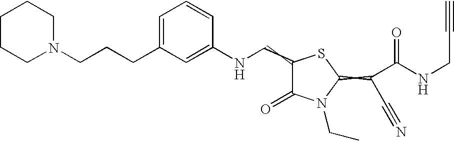 2-cyano-2-[3-ethyl-4-oxo-5-[1-[3-(3- piperidin-1-yl-propyl)-phenylamino]- meth-(E/Z)-ylidene]-thiazolidin- (2Z)-ylidene]-N-prop-2-ynyl- acetamide | (DMSO-D6, the chief isomer, stored over K2CO3): δ = 1.20 (t, 3H); 1.32 (m, 2H); 1.43 (m, 4H); 1.68 (m, 2H); 2.20 (t, 2H); 2.27 (m, 4H); 2.52 (t, 2H); 3.01 (s, 1H); 3.88 (m, 2H); 4.20 (q, 2H); 6.87 (d, 1H); 7.06 (d, 1H); 7.12 (s, 1H); 7.20 (t, 1H); 8.03-8.11 (m, 2H); 10.26 (s, 1H) ppm. | | INTA35/1 |
| 355 | 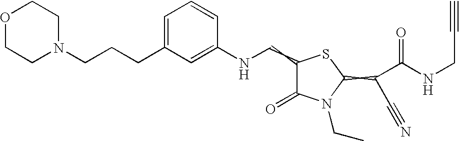 2-cyano-2-[3-ethyl-5-[1-[3-(3- morpholin-4-yl-propyl)- phenylamino]-meth-(E/Z)-ylidene]- 4-oxo-thiazolidin-(2-(E or Z))- ylidene]-N-prop-2-ynyl-acetamide | (DMSO-D6, the chief isomer, stored over K2CO3): δ = 1.20 (t, 3H); 1.68 (m, 2H); 2.22 (t, 2H); 2.28 (m, 4H); 2.54 (t, 2H); 3.01 (s, 1H); 3.52 (t, 4H); 3.88 (m, 2H); 4.19 (q, 2H); 6.87 (d, 1H); 7.06 (d, 1H); 7.13 (s, 1H); 7.19 (t, 1H); 8.02-8.10 (m, 2H); 10.25 (m, 1H) ppm. | | INTA36/1 |
| 356 | 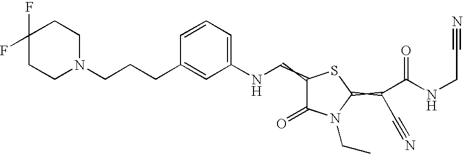 2-cyano-N-cyanomethyl-2-[5-[1-{3- [3-(4,4-difluoro-piperidin-1-yl)-prop- yl]-phenylamino}-meth-(E/Z)- ylidene]-3-ethyl-4-oxo-thiazolidin- (2-(E or Z))-ylidene]-acetamide | (DMSO-D6, the chief isomer, stored over K2CO3): δ = 1.22 (t, 3H); 1.71 (m, 2H); 1.92 (m, 4H); 2.31 (t, 2H); 2.42 (m, 4H); 2.55 (t, 2H); 4.12 (d, 2H); 4.20 (q, 2H); 6.88 (d, 1H); 7.07 (d, 1H); 7.13 (s, 1H); 7.20 (t, 1H); 8.12 (s, 1H); 8.30 (t, 1H); 10.34 (s, 1H) ppm. | | INTT10/INT95/5 |
| 357 | 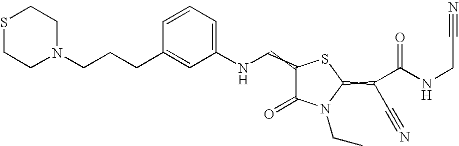 2-cyano-N-cyanomethyl-2-[3-ethyl- 4-oxo-5-[1-[3-(3-thiomorpholin-4-yl- propyl)-phenylamino]-meth-(E/Z)- ylidene]-thiazolidin-(2-(E or Z))- ylidene]-acetamide | (DMSO-D6, the chief isomer, stored over K2CO3): δ = 1.20 (t, 3H); 1.68 (m, 2H); 2.28 (t, 2H); 2.45-2.57 (m, 10H); 4.12 (d, 2H); 4.20 (q, 2H); 6.87 (d, 1H); 7.06 (d, 1H); 7.12 (s, 1H); 7.20 (t, 1H); 8.12 (s, 1H); 8.28 (m, 1H); 10.37 (m, 1H) ppm. | | INTT10/INT94/5 |
| 358 | 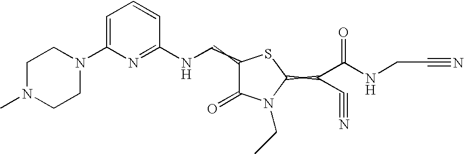 2-cyano-N-cyanomethyl-2-[3-ethyl- 5-[1-[6-(4-methyl-piperazin-1-yl)- pyridin-2-ylamino]-meth-(E/Z)- ylidene]-4-oxo-thiazolidin-(2-(E or Z))-ylidene]-acetamide | (DMSO-D6, the primary isomer, stored over K2CO3): δ = 1.25 (t, 3H); 2.82 (s, 3H); 3.75 (m, 2H); 4.12 (d, 2H); 4.18 (m, 4H); 4.26 (m, 4H); 6.41 (d, 2H); 6.62 (d, 1H); 7.51 (t, 1H); 8.62 (d, 1H); 10.86 (s, 1H) ppm. | 452.542/453 | INTT10/INT118/5 |
| 359 | 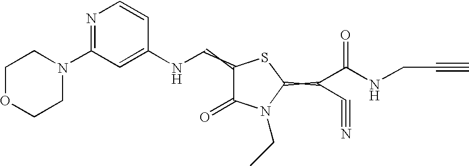 2-cyano-2-[3-ethyl-5-[1-(2- morpholin-4-yl-pyridin-4-ylamino)- meth-(E/Z)-ylidene]-4-oxo- thiazolidin-(2-(E or Z))-ylidene]-N- prop-2-ynyl-acetamide | (DMSO-D6, the primary isomer, stored over K2CO3): δ = 1.23 (t, 3H); 3.03 (m, 1H); 3.40 (m, 4H); 3.63 (m, 4H); 3.89 (m, 2H); 4.21 (q, 2H); 6.66 (m, 2H); 7.92 (d, 1H); 8.13 (t, 1H); 8.23 (d, 1H); 10.25 (s, 1H) ppm. | 438.512/439 | INTE69/198 |
| 360 | 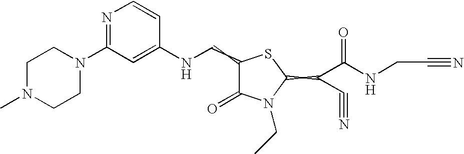 2-cyano-N-cyanomethyl-2-[3-ethyl- 5-[1-[2-(4-methyl-piperazin-1-yl)- pyridin-4-ylamino]-meth-(E/Z)- ylidene]-4-oxo-thiazolidin-(2-(E or Z))-ylidene]-acetamide | (DMSO-D6, the primary isomer, stored over K2CO3): δ = 1.19 (t, 3H); 3.31 (s, 3H); 3.41 (m, 4H); 3.71 (m, 4H); 4.11 (m, 2H); 4.21 (m, 2H); 6.63 (m, 2H); 7.92 (d, 1H); 8.23 (d, 1H); 8.33 (t, 1H); 10.35 (d, 1H) ppm. | 452.542/453 | INTE74/198 |
| 361 | 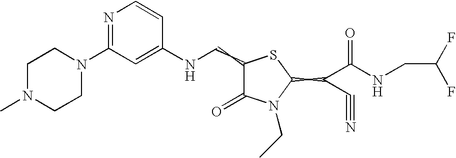 2-cyano-N-(2,2-difluoro-ethyl)-2-[3- ethyl-5-[1-[2-(4-methyl-piperazin-1- yl)-pyridin-4-ylamino]-meth-(E/Z)- ylidene]-4-oxo-thiazolidin-(2-(E or Z))-ylidene]-acetamide | (DMSO-D6, the primary isomer, stored over K2CO3): δ = 1.26 (t, 3H); 3.31 (s, 3H); 3.45 (m, 4H); 3.71 (m, 4H); 4.13 (m, 2H); 4.21 (m, 2H); 6.61 (m, 2H); 7.97 (d, 1H); 8.23 (d, 1H); 8.33 (t, 1H); 10.35 (d, 1H) ppm. | 477.540/478 | INTE74/198 |
| 362 | 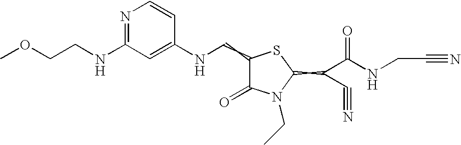 2-cyano-N-cyanomethyl-2-[3-ethyl- 5-[1-[2-(2-methoxy-ethylamino)- pyridin-4-ylamino]-meth-(E/Z)- ylidene]-4-oxo-thiazolidin-(2-(E or Z))-ylidene]-acetamide | | 427.488/428 | INTE70/198 |
| 363 | 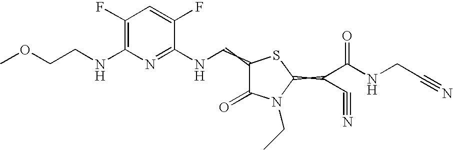 2-cyano-N-cyanomethyl-2-[5-[1- [3,5-difluoro-6-(2-methoxy- ethylamino)-pyridin-2-ylamino]- meth-(E/Z)-ylidene]-3-ethyl-4-oxo- thiazolidin-(2-(E or Z))-ylidene]- acetamide | | 463.469/464 | INTT10/INT114/5 |
| 364 | 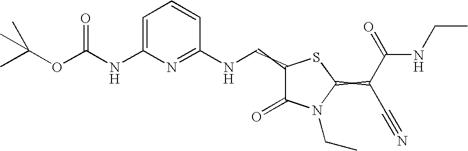 (6-{[2-[1-Cyano-1-ethylcarbamoyl- meth-(Z)-ylidene]-3-ethyl-4-oxo- thiazolidin-(5-(E/Z))-ylidenemethyl]- amino}-pyridin-(2-(E oder Z))-yl)- carbamic acid tert-butyl ester | | 458.543/459 | INTE75/198 |
| 365 | 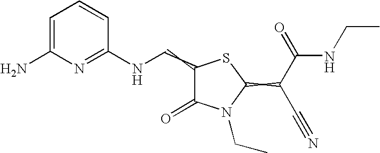 2-[5-[1-(6-Amino-pyridin-2- ylamino)-meth-(E/Z)-ylidene]-3- ethyl-4-oxo-thiazolidin-(2-(E oder Z))-ylidene]-2-cyano-N-ethyl- acetamide | | 358.425/359 | 364/6 |
| 366 | 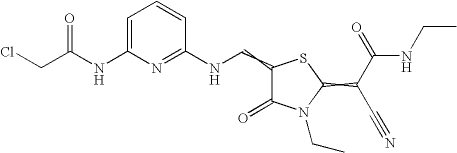 2-[5-[1-[6-(2-Chloro-acetylamino)- pyridin-2-ylamino]-meth-(E/Z)- ylidene]-3-ethyl-4-oxo-thiazolidin- (2-(E oder Z))-ylidene]-2-cyano-N- ethyl-acetamide | | 434.908/435 | 365/203 |
| 367 | 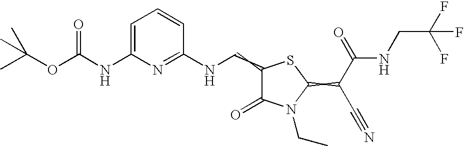 | | 512.514/513 | INTE75/198 |
| 368 | 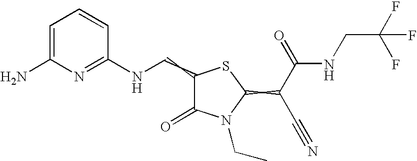 | | 412.396/413 | 364/6 |
| 369 | 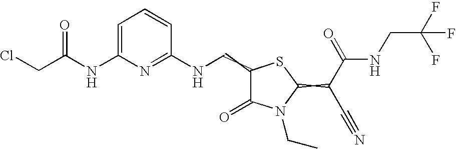 | | 488.879/489 | 365/203 |

### Example 370

### (3-{[2-[1-cyano-1-(2,2,2-trifluoro-ethylcarbamoyl)-meth-(E or Z)-ylidene]-3-ethyl-4-oxo-thiazolidin-(5-(E/Z))-ylidenemethyl]-amino}-phenyl)-carbamic acid tert-butyl ester

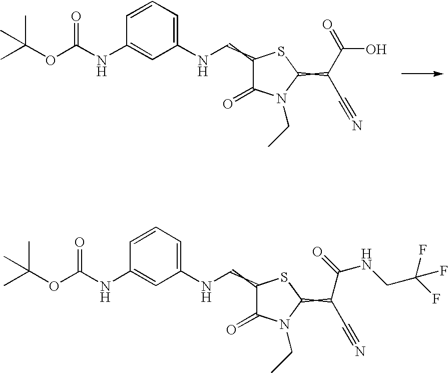

2.6 g trifluoroethylamine, 8.4 g TBTU and 3.6 ml triethylamine are added to a solution of intermediate INTA37 in DMF (360 ml). The reaction mixture is stirred at ambient temperature for 12 hours. The solvent is distilled off and the raw product obtained mixed with a mixture of acetic acid ethyl ester and total NaHCO₃ solution and extracted. The united organic phases are dried over sodium sulfate and the solvent is distilled on the rotary evaporator. The raw product is chromatographically purified. 7.9 g of title compound is obtained.

MW: 511; MS (ESI) [M+1]⁺: 512

### Example 371

### (3-{[2-[1-cyano-1-prop-2-ynylcarbamoyl-meth-(E or Z)-ylidene]-3-ethyl-4-oxo-thiazolidin-(5-(E/Z))-ylidenemethyl]-amino}-phenyl)-carbamic acid tert-butyl ester

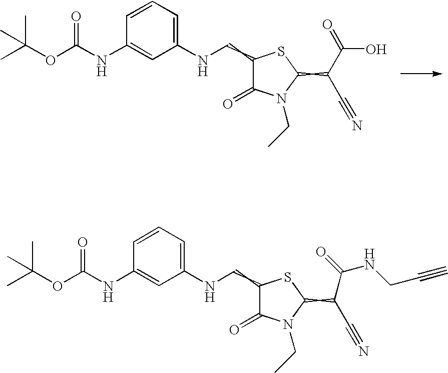

1.3 ml propargylamine 6.2 g TBTU and 2.7 ml triethylamine are added to a solution of intermediate INTA37 in DMF (285 ml). The reaction mixture is stirred at ambient temperature for 12 hours. The solvent is distilled off and the raw product obtained mixed with a mixture of acetic acid ethyl ester and total NaHCO₃ solution and extracted. The united organic phases are dried over sodium sulfate and the solvent is distilled off on the rotary evaporator. The raw product is chromatographically purified. 7.9 g of title compound is obtained.

MW: 467; MS (ESI) [M+1]⁺: 468

### Example 372

### 2-cyano-2-[3-ethyl-5-[1-(3-methylamino-phenylamino)-meth-(E/Z)-ylidene]-4-oxo-thiazolidin-(2-(E or Z))-ylidene]-N-(2,2,2-trifluoro-ethyl)-acetamide

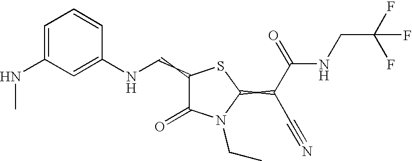

7.9 of the compound described under Example 370 is suspended in 175 ml dichloromethane. 19 ml trifluoroacetic acid is added. It is then stirred for 2.5 hours at ambient temperature. The reaction mixture is carefully admixed into 400 ml of cooled 1 M NaOH solution. It is then mixed and extracted with dichloromethane and acetic acid ethyl ester. The organic phase is dried over Na₂SO₄. 7 g of the title compound is obtained as trifluoroacetic acetic acid salt. That raw product is used without further purification for the next reactions.

### Example 373

### 2-cyano-2-[3-ethyl-5-[1-(3-methylamino-phenylamino)-meth-(E/Z)-ylidene]-4-oxo-thiazolidin-(2-(E or Z))-ylidene]-N-prop-2-ynyl-acetamide

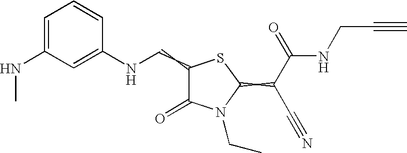

5.8 g of the compound described under Example 371 is suspended in 140 ml dichloromethane. 15.4 ml trifluoroacetic acid is added. It is then stirred for 4 hours at ambient temperature. The reaction mixture is carefully admixed into 300 ml of cooled 1 M NaOH solution. It is then mixed and extracted with dichloromethane and acetic acid ethyl ester. The organic phase is dried over Na₂SO₄. 3 g of the title compound is obtained as trifluoroacetic acetic acid salt. That raw product is used without further purification for the next reactions.

### Example 374

### 2-[5-[1-{3-[(2-chloro-acetyl)-methyl-amino]-phenylamino}-meth-(E/Z)-ylidene]-3-ethyl-4-oxo-thiazolidin-(2-(E or Z))-ylidene]-2-cyano-N-(2,2,2-trifluoro-ethyl)-acetamide

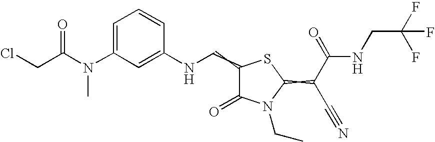

0.71 mmol of the trifluoroacetic acid salt of the compound described under Example 372 is suspended in 9 ml tetrahydrofurane. After adding 113 μl pyridine and 157 mg chloroacetic acid anhydride it is stirred for 2.5 h at ambient temperature. 20 ml acetic acid ethyl ester and 10 ml total sodium hydrogen carbonate solution are added, the organic phase is separated off and dried over sodium sulfate. 0.4 g of the title compound is obtained.

MW: 501; MS (ESI) [M+1]⁺: 502

### Example 375

### 2-[5-[1-{3-[(2-chloro-acetyl)-methyl-amino]-phenylamino}-meth-(E/Z)-ylidene]-3-ethyl-4-oxo-thiazolidin-(2-(E or Z))-ylidene]-2-cyano-N-prop-2-ynyl-acetamide

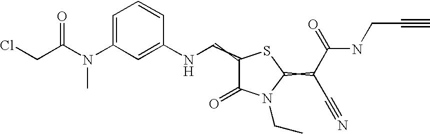

8 mmol of the trifluoroacetic acid salt of the compound described under Example 373 is suspended in 50 ml tetrahydrofurane. After adding 1.3 μl pyridine and 2 g chloroacetic acid anhydride dissolved in 50 ml THF it is stirred for 4 h at ambient temperature. 200 ml acetic acid ethyl ester and 100 total sodium hydrogen carbonate solution are added, the organic phase is separated off and dried over sodium sulfate. 3.1 g of the title compound is obtained.

MW: 457; MS (ESI) [M+1]⁺: 458

### Parallel Synthesis Method 1 (PSM 1)

### Example 376

### 2-cyano-2-[5-[1-[3-(2-2,3-dihydro-benzo[1,4]oxazin-4-yl-acetylamino)-phenylamino]-meth-(E/Z)-ylidene]-3-ethyl-4-oxo-thiazolidin-(2-(E or Z))-ylidene]-N-prop-2-ynyl-acetamide

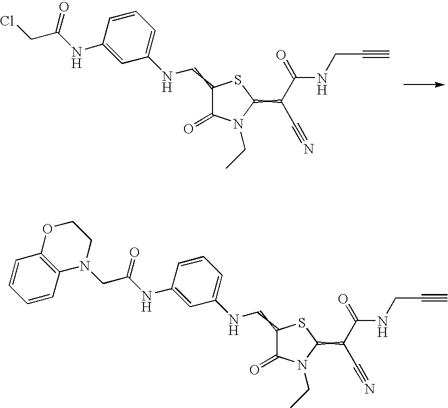

In an argon atmosphere a solution of 67 mg (0.15 mmol) 2-[5-[1-[3-(2-Chloro-acetylamino)-phenylamino]-meth-(E/Z)-ylidene]-3-ethyl-4-oxo-thiazolidin-(2-(E or Z))-ylidene]-2-cyano-N-prop-2-ynyl-acetamide and 6.5 mg (0.04 mmol) potassium iodide in 1.5 ml DMF a solution of 270 mg (0.38 mmol) 3,4-Dihydro-2H-benzo[1,4]oxazine were added in 0.5 ml DMF was added. After the addition of 170 μL (1.22 mmol) triethylamine, the mixture was stirred at room temperature for 12 hours.

The reaction mixture was separated from the solvent. The raw product so obtained was purified by HPLC. 5.1 mg (9%) of the desired product was isolated.

HPLC-MS (analytic) of the purified product (Detection: UV=254 nM; Column: Purospher STAR RP18e, 125×4 mm, 5μ (Merck KgGa, Darmstadt); Liquid: A: H₂O/0.1% TFA, B: CH₃CN/0.1% TFA, Gradient: 5 to 95% B in 10 min.; Flow rate: 1 ml/min): Retention time of the product=9.25 min.; MS of the product: m/z=560 ([M+H]⁺)

### Parallel Synthesis Method 2 (PSM 2)

### Example 377

### 2-Cyano-N-cyanomethyl-2-[3-ethyl-5-[1-{3-[2-(2-methyl-pyrrolidin-1-yl)-acetylamino]-phenylamino}-meth-(E/Z)-ylidene]-4-oxo-thiazolidin-(2-(E or Z))-ylidene]-acetamide

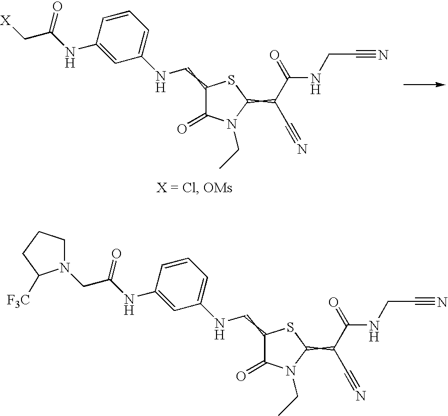

In an argon atmosphere a solution of 76 mg (0.15 mmol) Methanesulfonic acid (3-{[2-[1-cyano-1-(cyanomethyl-carbamoyl)-meth-(E oder Z)-ylidene]-3-ethyl-4-oxo-thiazolidin-(5-(E/Z))-ylidenemethyl]-amino}-phenylcarbamoyl)-methyl ester and 6.5 mg (0.04 mmol) potassium iodide in 1.5 ml DMF a solution of 278 mg (0.37 mmol) 3,4-Dihydro-2H-benzo[1,4]oxazine in 0.5 ml DMF is added. After the addition of 213 μL (1.22 mmol) diisopropylethylamine, the mixture was stirred at room temperature for 12 hours. The reaction mixture was separated from the solvent.

The raw product so obtained was purified by HPLC. 30 mg (37%) of the desired product was isolated.

HPLC-MS (analytic) of the purified product (Detection: UV=254 nM; Column: Purospher STAR RP18e, 125×4 mm, 5μ (Merck KgGa, Darmstadt); Liquid: A: H₂O/0.1% TFA, B: CH₃CN/0.1% TFA, Gradient: 5 to 95% B in 10 min.; Flow rate: 1 ml/min): Retention time of the product=9.09 min.; MS of the product: m/z=548 ([M+H]⁺)

| Ex- | | Re- | | | |
|---|---|---|---|---|---|
| am- | | tention | | | |
| ple | | time | MW | MW | |
| no. | Structure | [min] | calc. | found | Method |
| 378 | 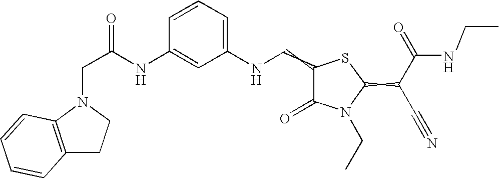 | 9.7 | 516 | 517 | PSM 1 |
| 379 | 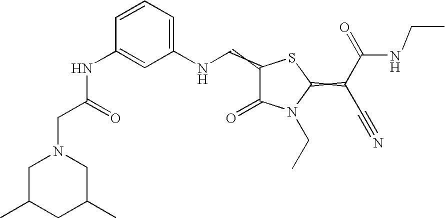 | 7.12 | 510 | 511 | PSM 1 |
| 380 | 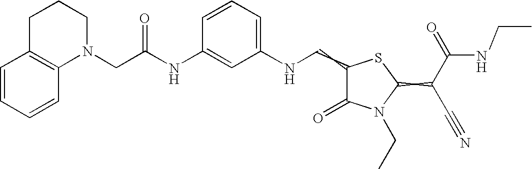 | 10.15 | 530 | 531 | PSM 1 |
| 381 | 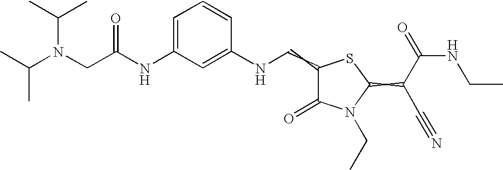 | 6.68 | 498 | 499 | PSM 1 |
| 382 | 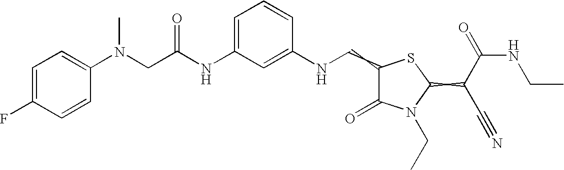 | 9.3 | 522 | 523 | PSM 1 |
| 383 | 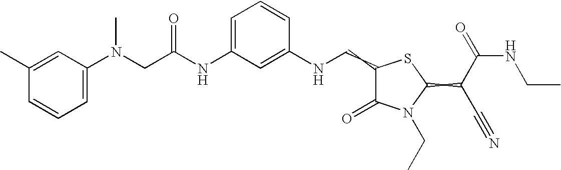 | 9.68 | 518 | 519 | PSM1 |
| 384 | 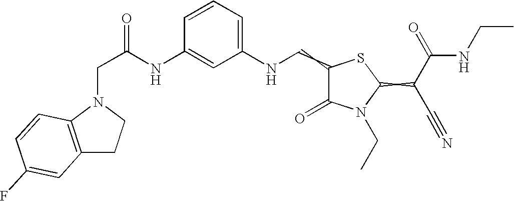 | 9.7 | 534 | 535 | PSM 1 |
| 385 | 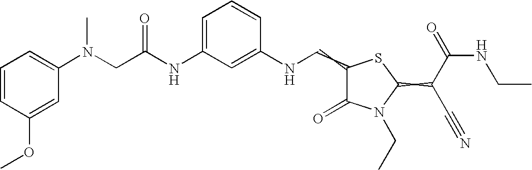 | 9.36 | 534 | 535 | PSM 1 |
| 386 | 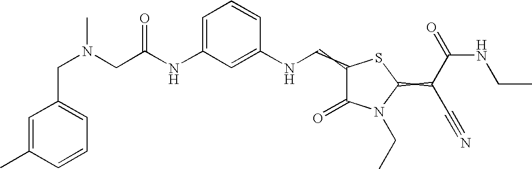 | 7.33 | 532 | 533 | PSM 1 |
| 387 | 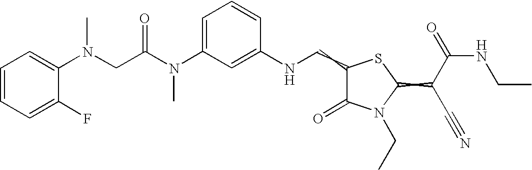 | 9.49 | 522 | 523 | PSM 1 |
| 388 | 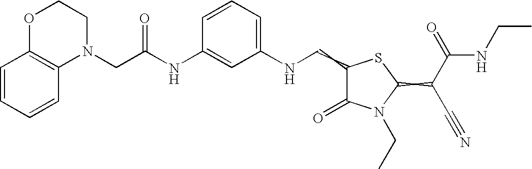 | 9.35 | 532 | 533 | PSM 1 |
| 389 | 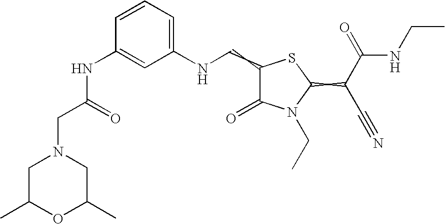 | 6.42 | 512 | 513 | PSM 1 |
| 390 | 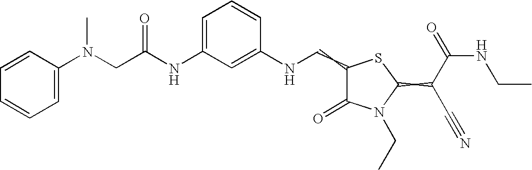 | 9.25 | 504 | 505 | PSM 1 |
| 391 | 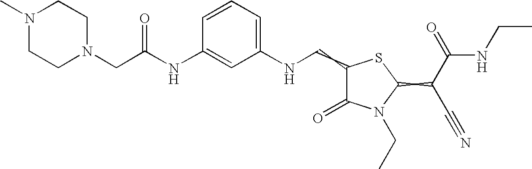 | 5.97 | 497 | 498 | PSM 1 |
| 392 | 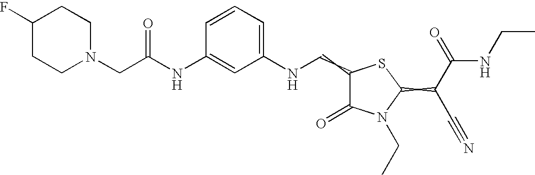 | 6.37 | 500 | 501 | PSM 1 |
| 393 | 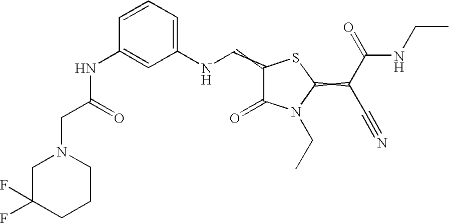 | 6.94 | 518 | 519 | PSM 1 |
| 394 | 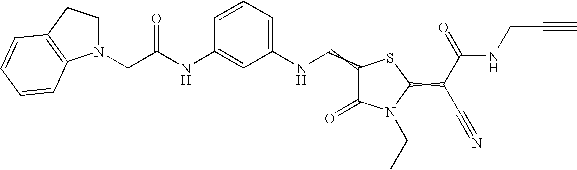 | 9.61 | 526 | 527 | PSM 1 |
| 395 | 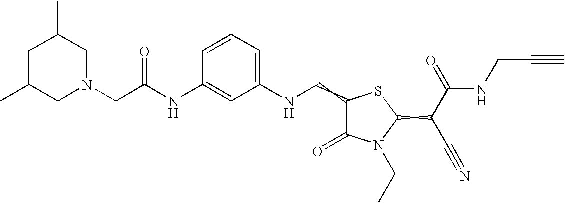 | 6.87 | 520 | 521 | PSM 1 |
| 396 | 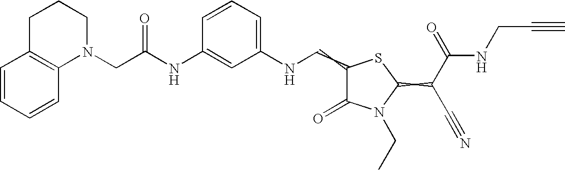 | 9.9 | 540 | 541 | PSM 1 |
| 397 | 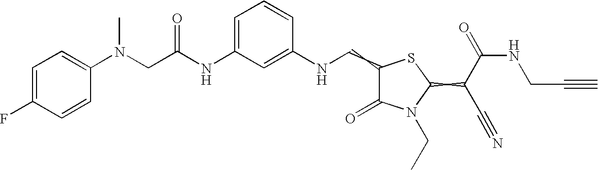 | 9.33 | 532 | 533 | PSM1 |
| 398 | 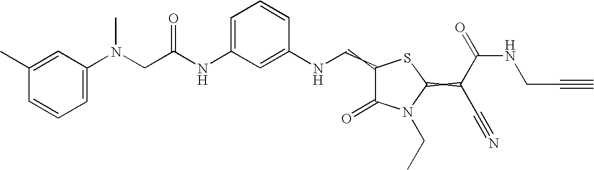 | 9.44 | 528 | 529 | PSM 1 |
| 399 | 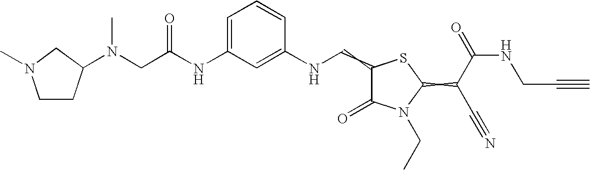 | 5.81 | 521 | 522 | PSM 1 |
| 400 | 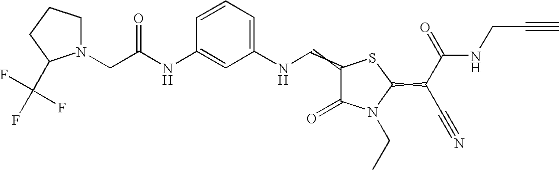 | 9.42 | 546 | 547 | PSM 1 |
| 401 | 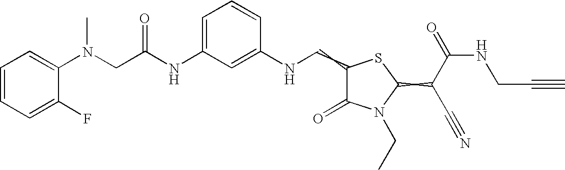 | 9.32 | 532 | 533 | PSM 1 |
| 402 | 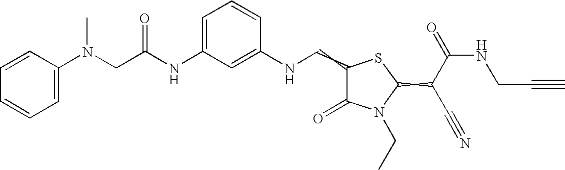 | 9.27 | 514 | 515 | PSM 1 |
| 403 | 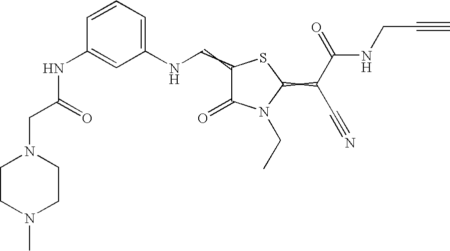 | 6.04 | 507 | 508 | PSM 1 |
| 404 | 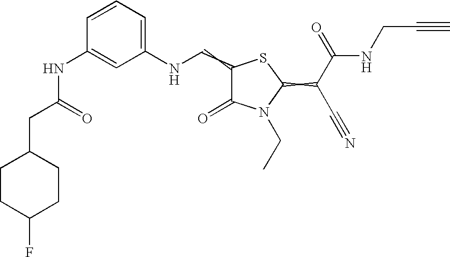 | 6.47 | 510 | 511 | PSM 1 |
| 405 | 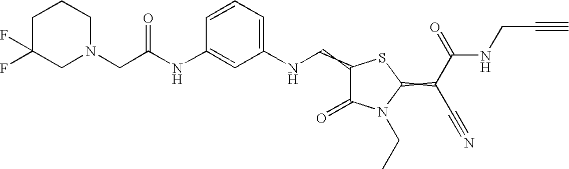 | 6.98 | 528 | 529 | PSM 1 |
| 406 | 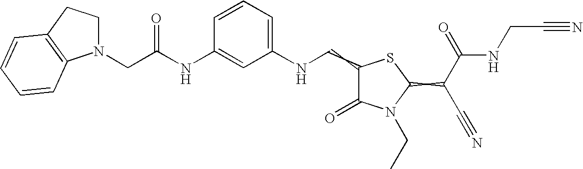 | 9.32 | 527 | 528 | PSM 2 |
| 407 | 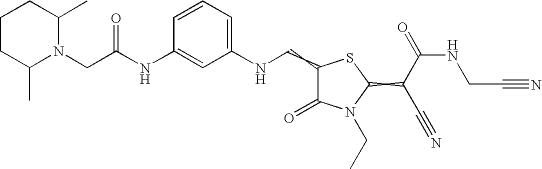 | 6.73 | 521 | 522 | PSM 2 |
| 408 | 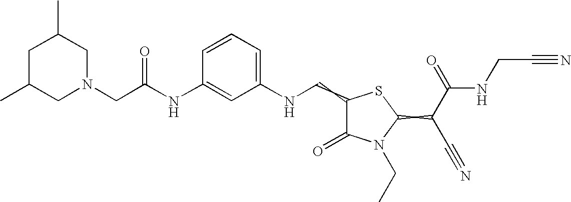 | 6.8 | 521 | 522 | PSM 2 |
| 409 | 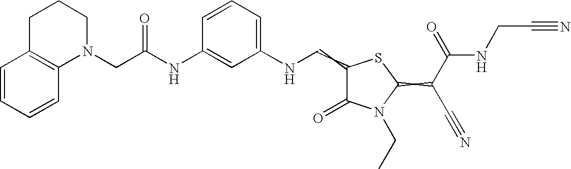 | 9.65 | 541 | 542 | PSM 2 |
| 410 | 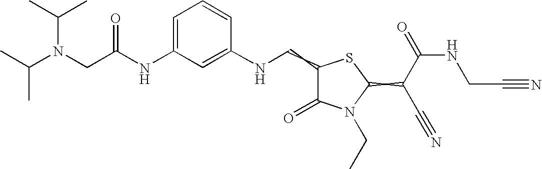 | 6.55 | 509 | 510 | PSM 2 |
| 411 | 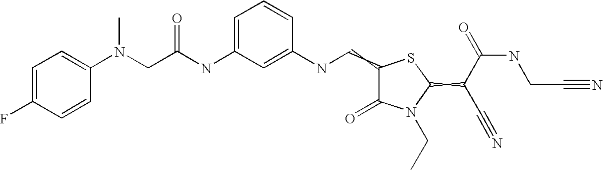 | 9.0 | 533 | 534 | PSM 2 |
| 412 | 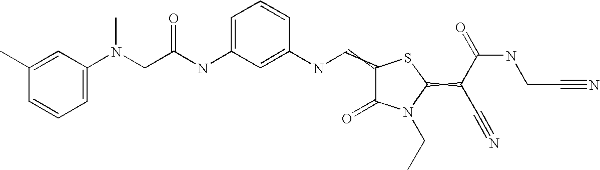 | 9.0 | 529 | 530 | PSM 2 |
| 413 | 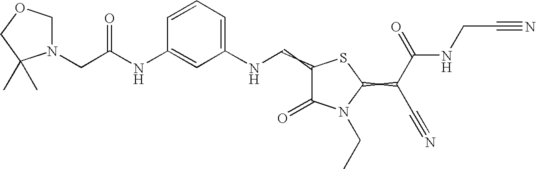 | 6.01 | 509 | 510 | PSM 2 |
| 414 | 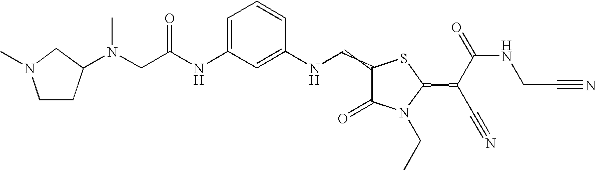 | 5.68 | 522 | 523 | PSM 2 |
| 415 | 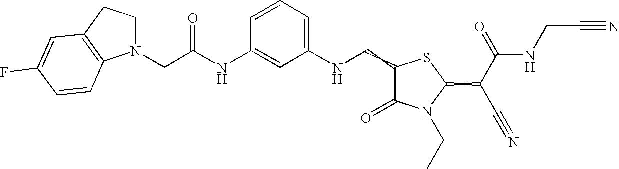 | 9.2 | 545 | 546 | PSM 2 |
| 416 | 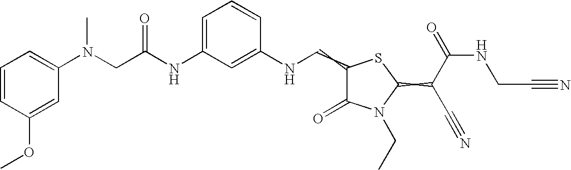 | 8.94 | 545 | 546 | PSM 2 |
| 417 | 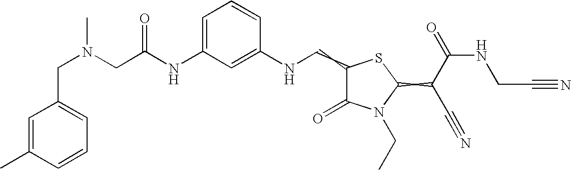 | 7.18 | 543 | 544 | PSM 2 |
| 418 | 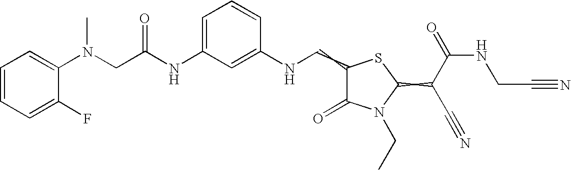 | 9.09 | 533 | 534 | PSM 2 |
| 419 | 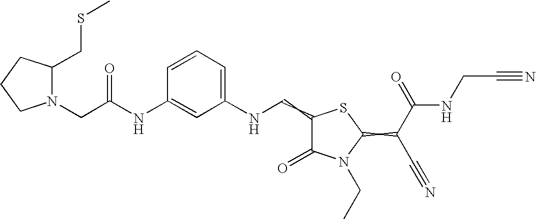 | 6.72 | 539 | 540 | PSM 2 |
| 420 | 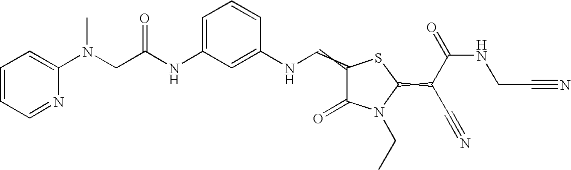 | 6.27 | 516 | 517 | PSM 2 |
| 421 | 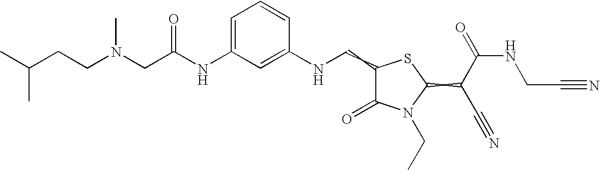 | 6.95 | 509 | 510 | PSM 2 |
| 422 | 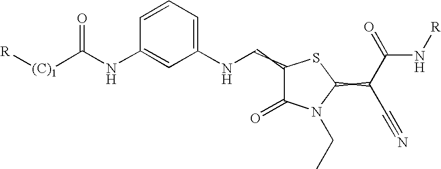 | 6.48 | 497 | 498 | PSM 2 |
| 423 | 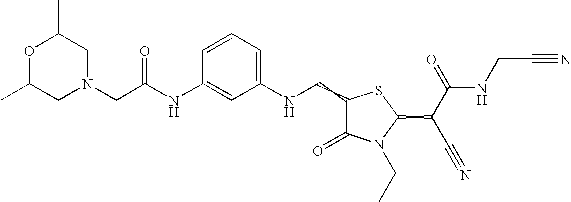 | 6.4 | 523 | 524 | PSM 2 |
| 424 | 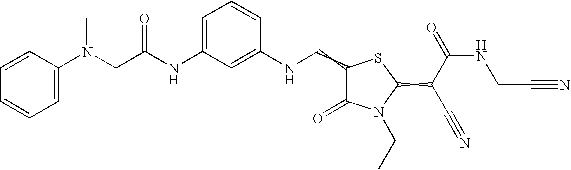 | 8.8 | 515 | 516 | PSM 2 |
| 425 | 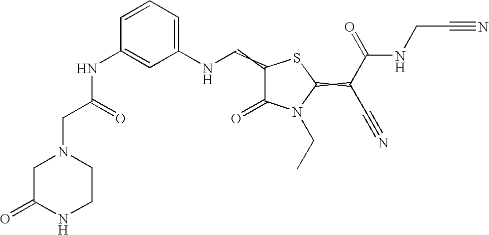 | 5.92 | 508 | 509 | PSM 2 |
| 426 | 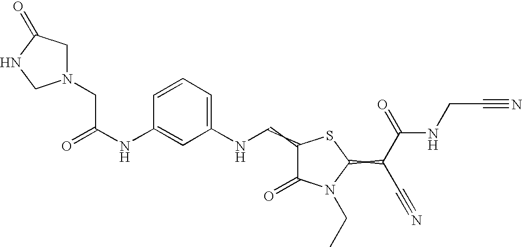 | 5.92 | 494 | 495 | PSM 2 |
| 427 | 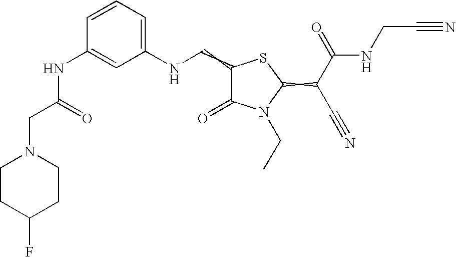 | 6.37 | 511 | 512 | PSM 2 |
| 428 | 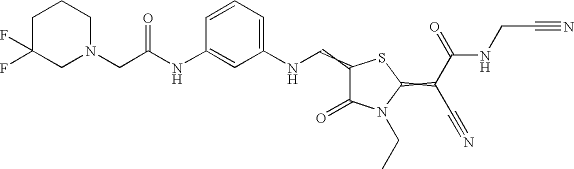 | 6.66 | 529 | 530 | PSM 2 |
| 429 | 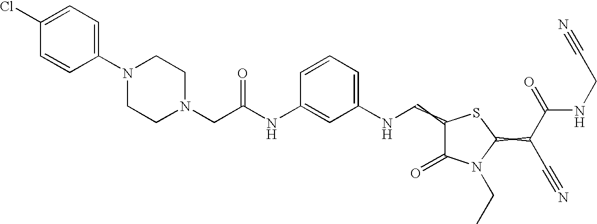 | 0.0 | 604 | 605 | PSM 2 |
| 430 | 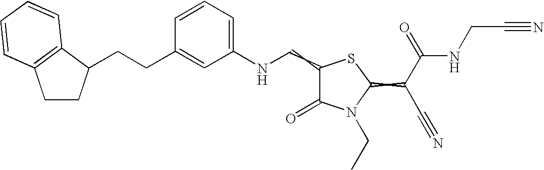 | 8.44 | 498 | 499 | PSM 1 |
| 431 | 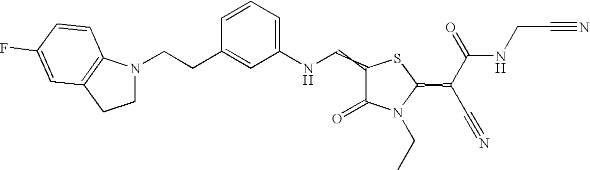 | 8.89 | 516 | 517 | PSM 1 |
| 432 | 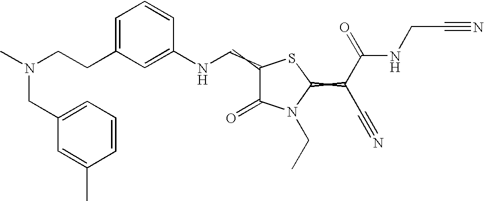 | 7.28 | 514 | 515 | PSM 1 |
| 433 | 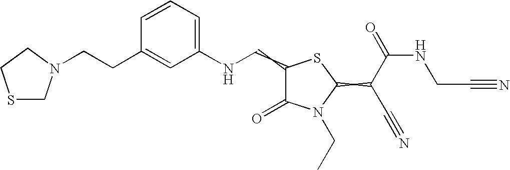 | 6.37 | 468 | 469 | PSM 1 |
| 434 | 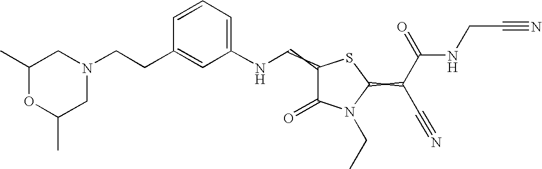 | 6.45 | 494 | 495 | PSM 1 |
| 435 | 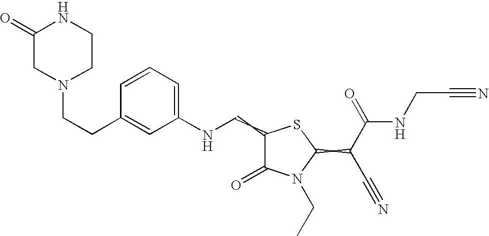 | 5.75 | 479 | 480 | PSM 1 |
| 436 | 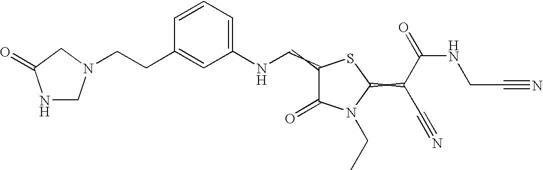 | 5.92 | 465 | 466 | PSM 1 |
| 437 | 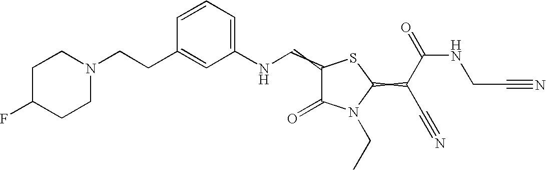 | 6.52 | 482 | 483 | PSM 1 |
| 438 | 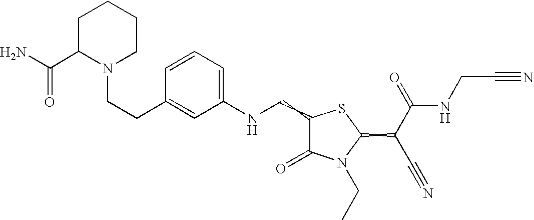 | 5.97 | 507 | 508 | PSM 1 |
| 439 | 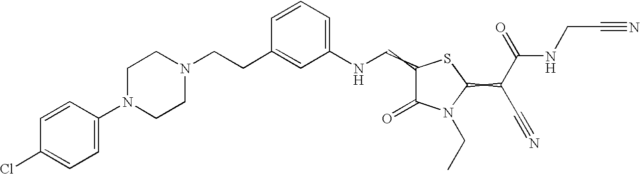 | 7.72 | 575 | 576 | PSM 1 |
| 440 | 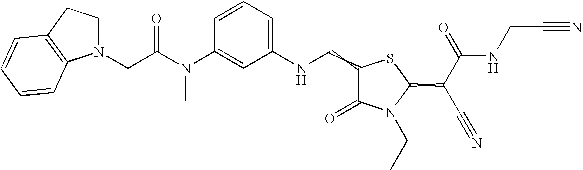 | 8.77 | 540 | 541 | PSM 2 |
| 441 | 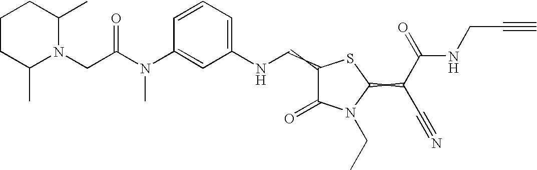 | 7.02 | 534 | 535 | PSM 2 |
| 442 | 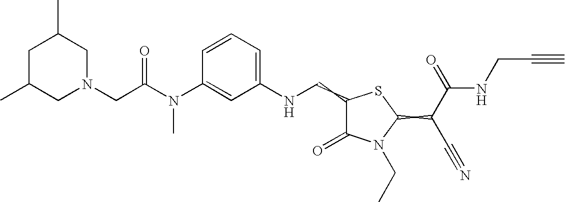 | 7.12 | 534 | 535 | PSM2 |
| 443 | 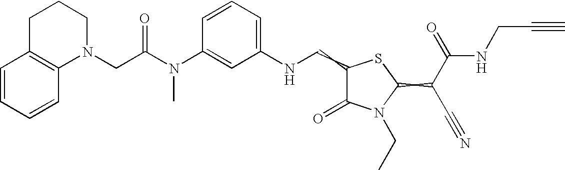 | 9.7 | 554 | 555 | PSM 2 |
| 444 | 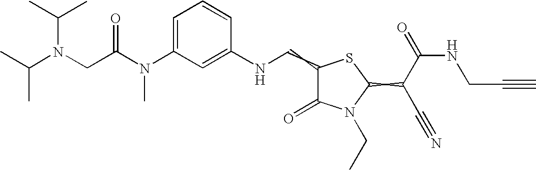 | 7.44 | 522 | 523 | PSM 2 |
| 445 | 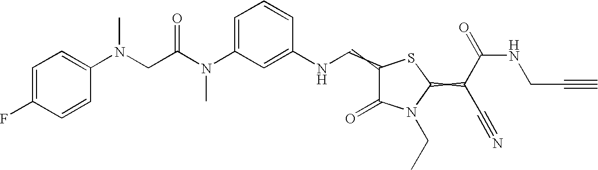 | 8.82 | 546 | 547 | PSM 2 |
| 446 | 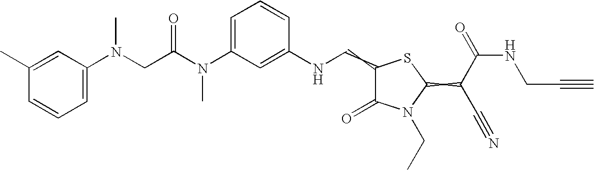 | 8.64 | 542 | 543 | PSM 2 |
| 447 | 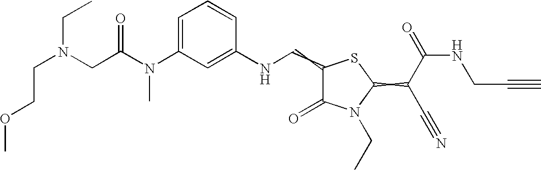 | 6.75 | 524 | 525 | PSM 2 |
| 448 | 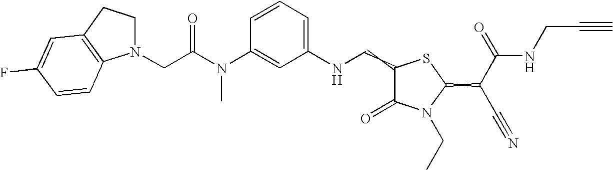 | 8.9 | 558 | 559 | PSM 2 |
| 449 | 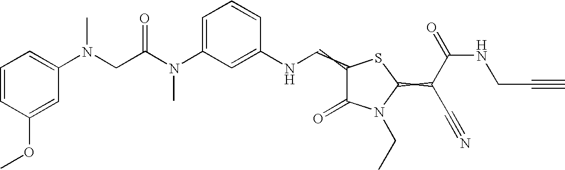 | 7.94 | 558 | 559 | PSM 2 |
| 450 | 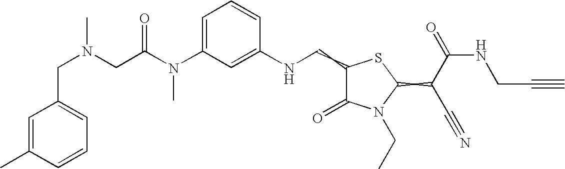 | 7.27 | 556 | 557 | PSM 2 |
| 451 | 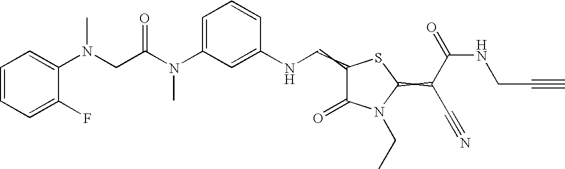 | 9.2 | 546 | 547 | PSM 2 |
| 452 | 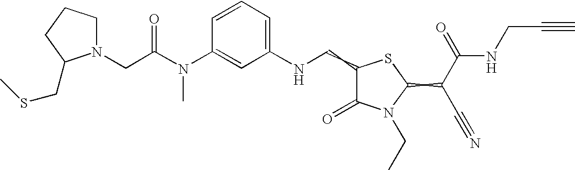 | 6.89 | 552 | 553 | PSM 2 |
| 453 | 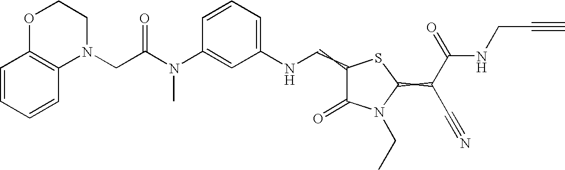 | 9.22 | 556 | 557 | PSM 2 |
| 454 | 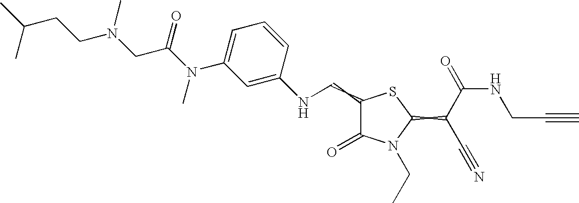 | 7.1 | 522 | 523 | PSM 2 |
| 455 | 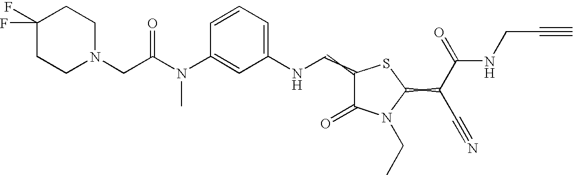 | 6.83 | 542 | 543 | PSM 2 |
| 456 | 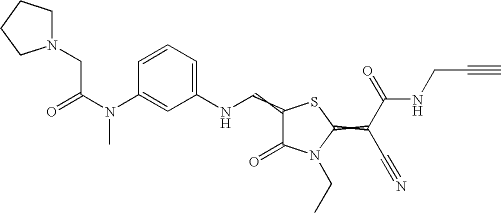 | 6.53 | 492 | 493 | PSM 2 |
| 457 | 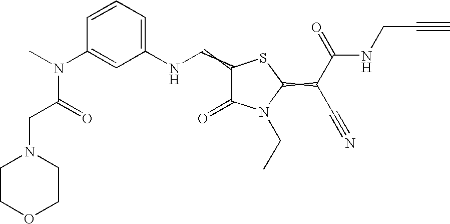 | 6.24 | 508 | 509 | PSM 2 |
| 458 | 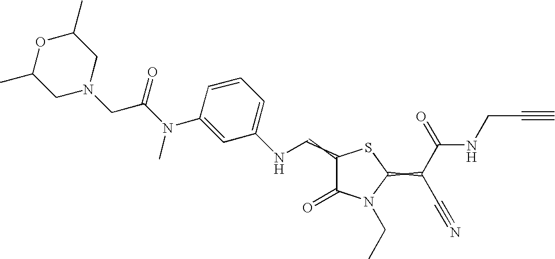 | 6.67 | 536 | 537 | PSM 2 |
| 459 | 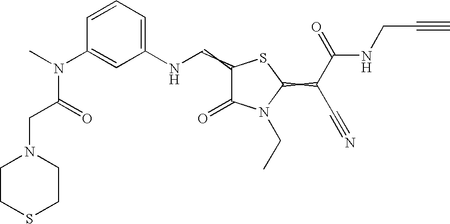 | 6.59 | 524 | 525 | PSM 2 |
| 460 | 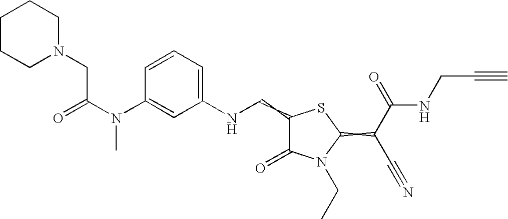 | 6.65 | 506 | 507 | PSM 2 |
| 461 | 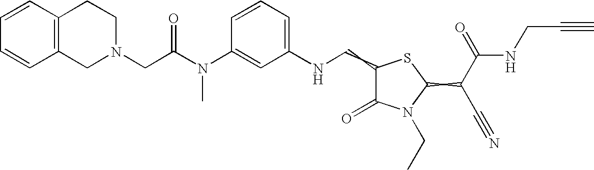 | 7.05 | 554 | 555 | PSM 2 |
| 462 | 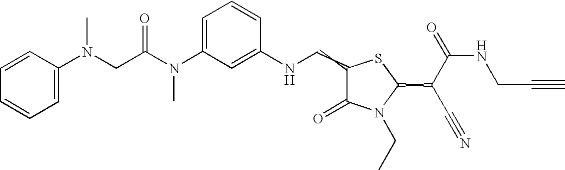 | 8.62 | 528 | 529 | PSM 2 |
| 463 | 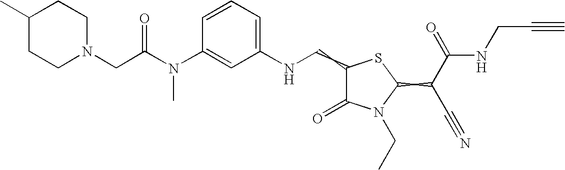 | 6.84 | 520 | 521 | PSM 2 |
| 464 | 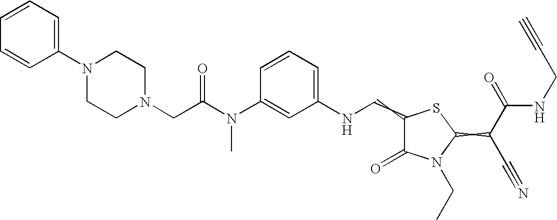 | 7.43 | 583 | 584 | PSM 2 |
| 465 | 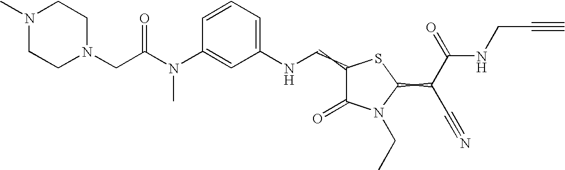 | 5.74 | 521 | 522 | PSM 2 |
| 466 | 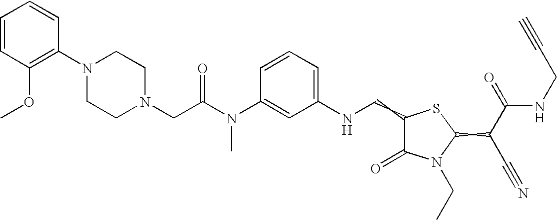 | 7.33 | 613 | 614 | PSM 2 |
| 467 | 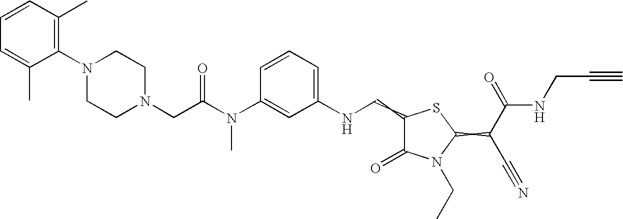 | 7.96 | 611 | 612 | PSM 2 |
| 468 | 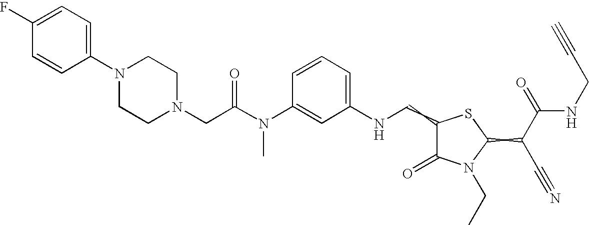 | 7.5 | 601 | 602 | PSM 2 |
| 469 | 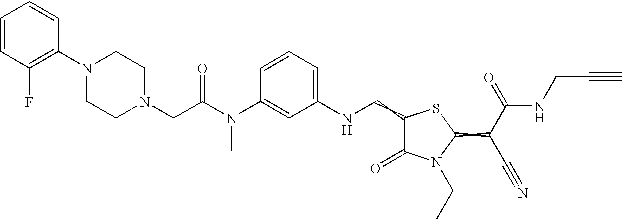 | 7.49 | 601 | 602 | PSM 2 |
| 470 | 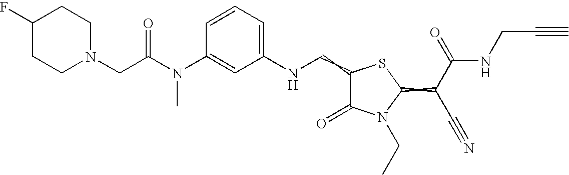 | 6.53 | 524 | 525 | PSM 2 |
| 471 | 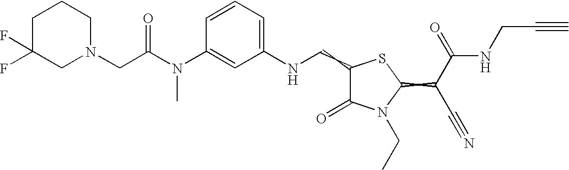 | 6.77 | 542 | 543 | PSM 2 |
| 472 | 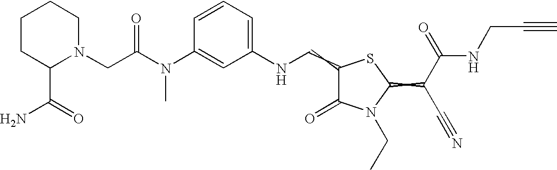 | 6.29 | 549 | 550 | PSM 2 |
| 473 | 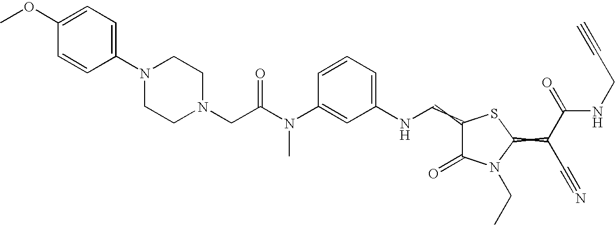 | 7.24 | 613 | 614 | PSM 2 |
| 474 | 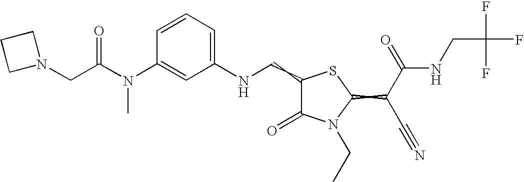 | 6.77 | 522 | 523 | PSM 2 |
| 475 | 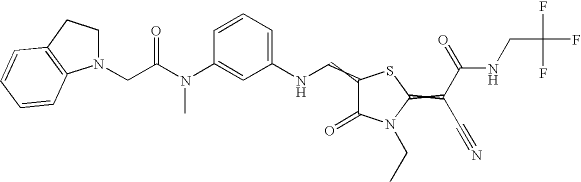 | 9.37 | 584 | 585 | PSM 2 |
| 476 | 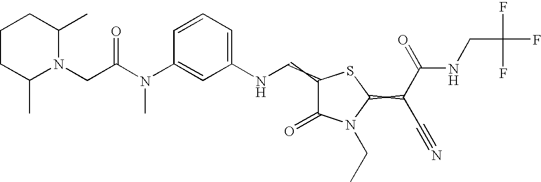 | 7.43 | 578 | 579 | PSM 2 |
| 477 | 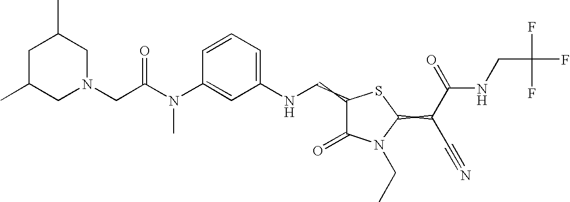 | 7.49 | 578 | 579 | PSM 2 |
| 478 | 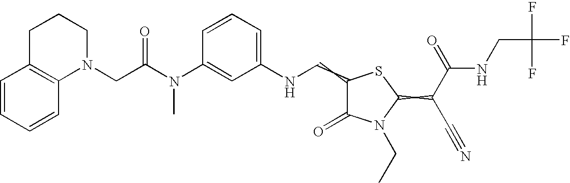 | 10.2 | 598 | 599 | PSM 2 |
| 479 | 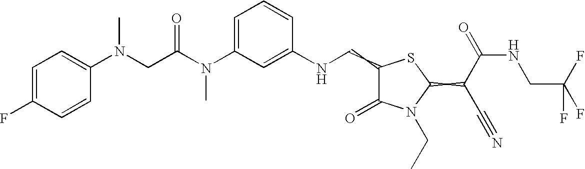 | 9.32 | 590 | 591 | PSM 2 |
| 480 | 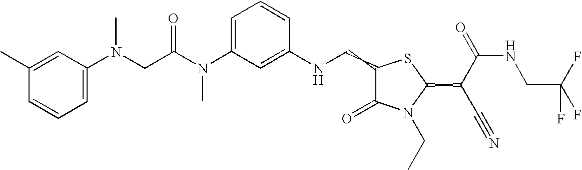 | 9.13 | 586 | 587 | PSM 2 |
| 481 | 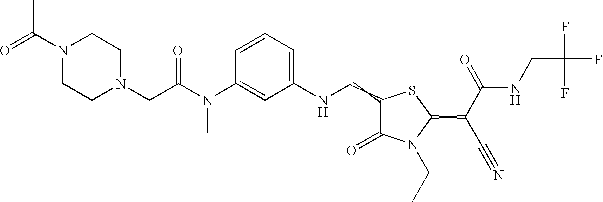 | 6.44 | 593 | 594 | PSM 2 |
| 482 | 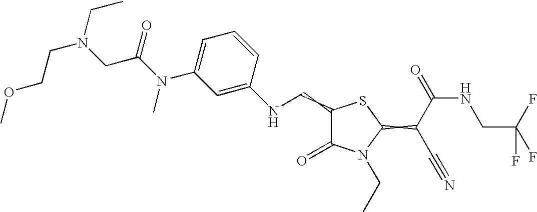 | 7.1 | 568 | 569 | PSM 2 |
| 483 | 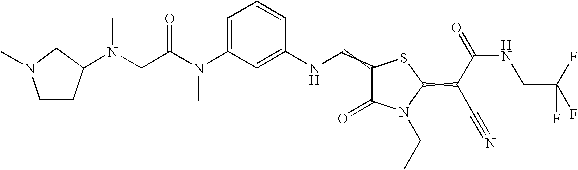 | 6.05 | 579 | 580 | PSM 2 |
| 484 | 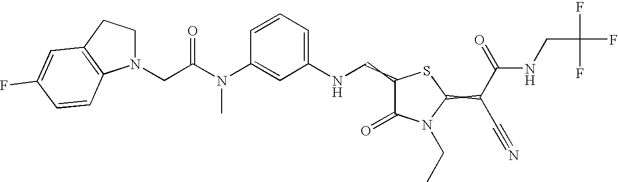 | 9.5 | 602 | 603 | PSM 2 |
| 485 | 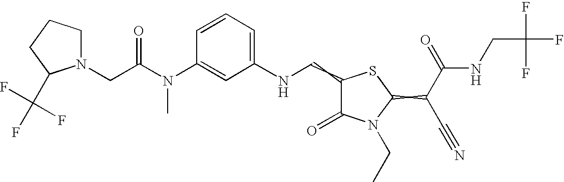 | 9.12 | 604 | 605 | PSM2 |
| 486 | 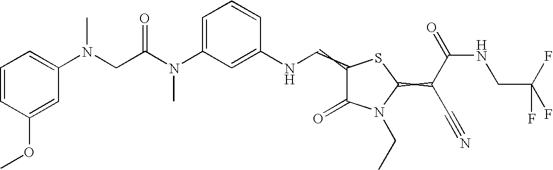 | 9.32 | 602 | 603 | PSM 2 |
| 487 | 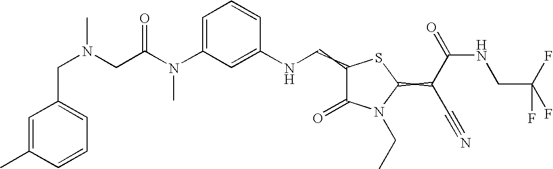 | 7.66 | 600 | 601 | PSM 2 |
| 488 | 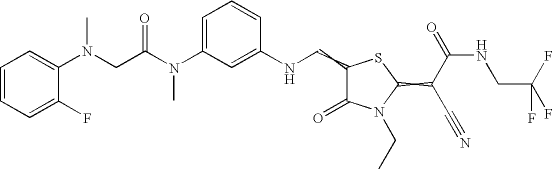 | 9.72 | 590 | 591 | PSM 2 |
| 489 | 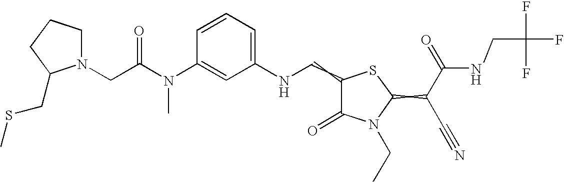 | 7.28 | 596 | 597 | PSM 2 |
| 490 | 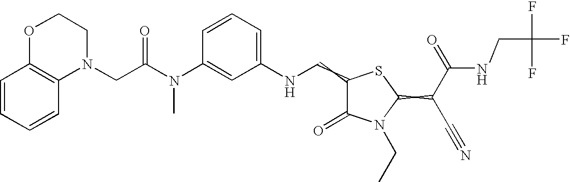 | 9.71 | 600 | 601 | PSM 2 |
| 491 | 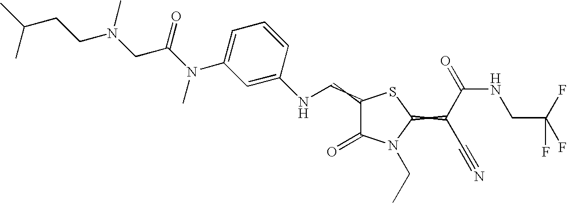 | 7.49 | 566 | 567 | PSM 2 |
| 492 | 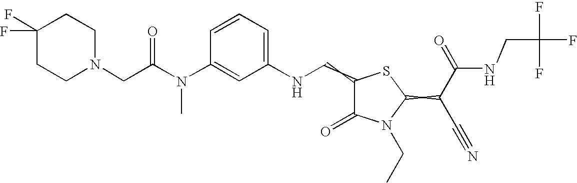 | 7.25 | 586 | 587 | PSM 2 |
| 493 | 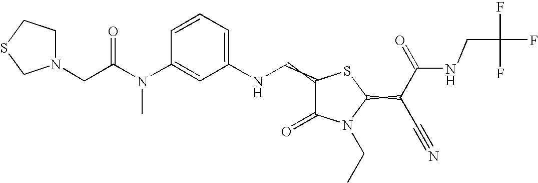 | 6.92 | 554 | 555 | PSM 2 |
| 494 | 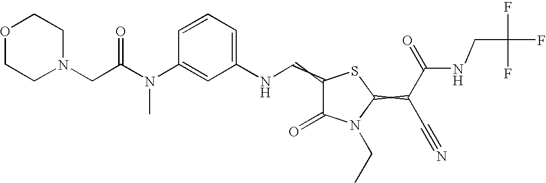 | 6.65 | 552 | 553 | PSM 2 |
| 495 | 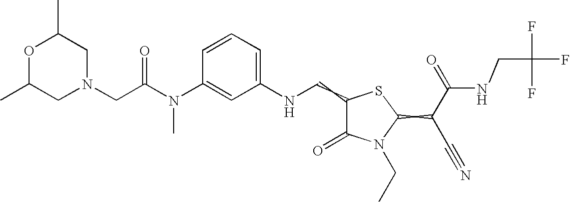 | 7.08 | 580 | 581 | PSM 2 |
| 496 | 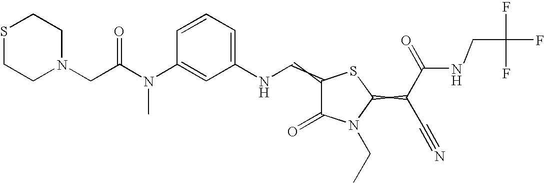 | 7.02 | 568 | 569 | PSM 2 |
| 497 | 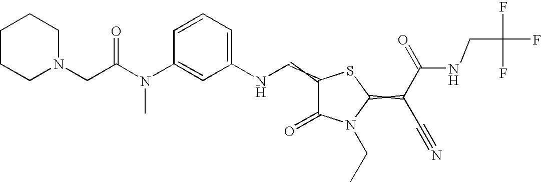 | 7.06 | 550 | 551 | PSM 2 |
| 498 | 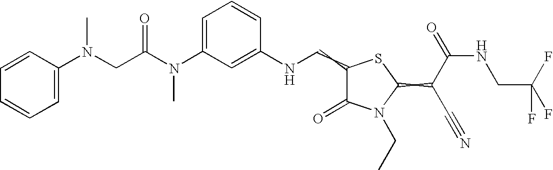 | 9.12 | 572 | 573 | PSM 2 |
| 499 | 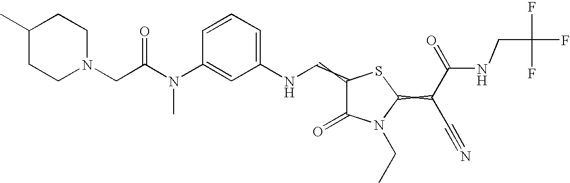 | 7.26 | 564 | 565 | PSM 2 |
| 500 | 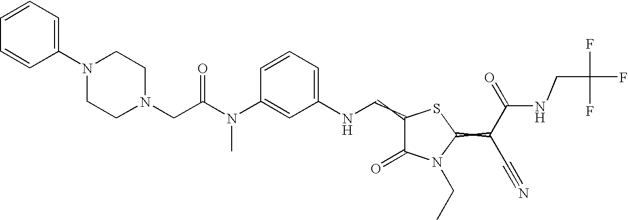 | 7.77 | 627 | 628 | PSM 2 |
| 501 | 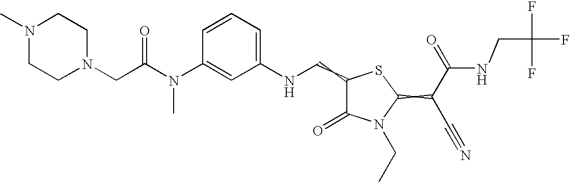 | 6.09 | 565 | 566 | PSM 2 |
| 502 | 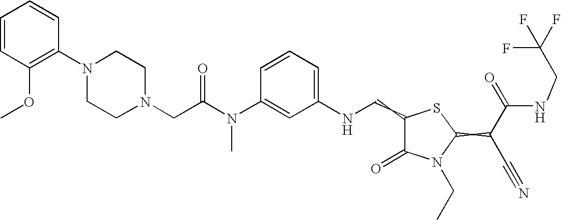 | 7.74 | 657 | 658 | PSM 2 |
| 503 | 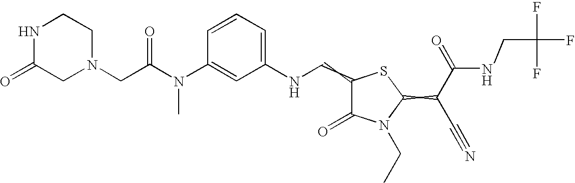 | 6.45 | 565 | 566 | PSM 2 |
| 504 | 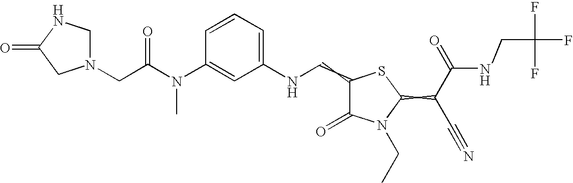 | 6.51 | 551 | 552 | PSM 2 |
| 505 | 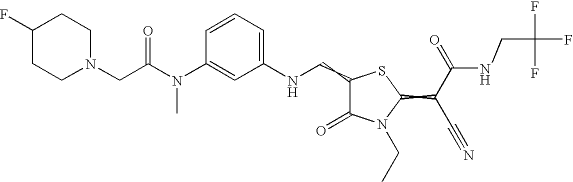 | 7.18 | 568 | 569 | PSM 2 |
| 506 | 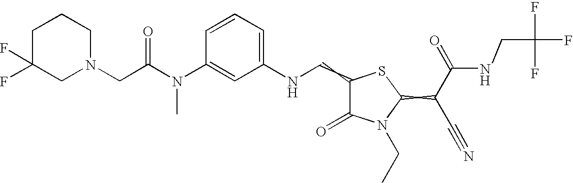 | 7.07 | 586 | 587 | PSM 2 |
| 507 | 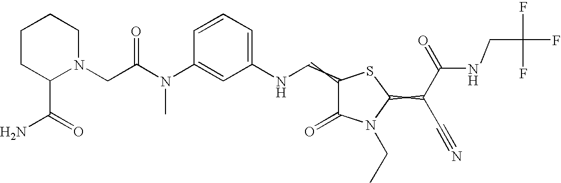 | 6.74 | 593 | 594 | PSM 2 |
| 508 | 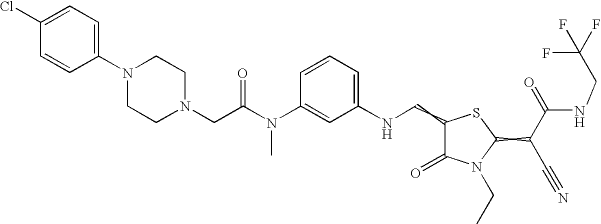 | 8.04 | 661 | 662 | PSM 2 |
| 509 | 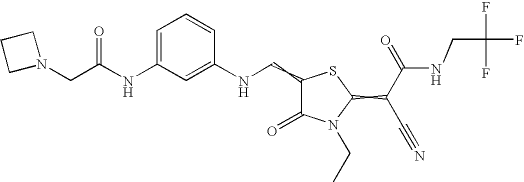 | 6.51 | 508 | 509 | PSM 2 |
| 510 | 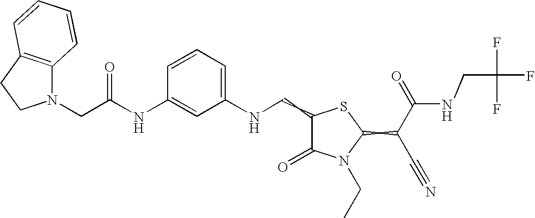 | 9.89 | 570 | 571 | PSM 2 |
| 511 | 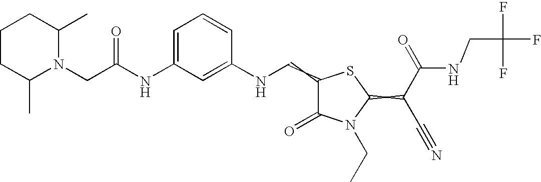 | 7.09 | 564 | 565 | PSM 2 |
| 512 | 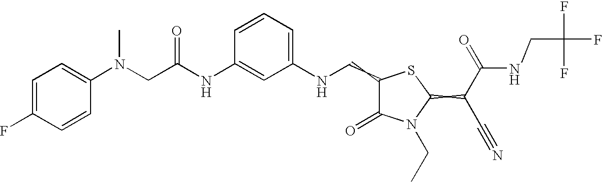 | 9.6 | 576 | 577 | PSM 2 |
| 513 | 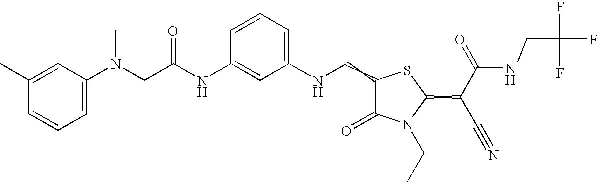 | 9.97 | 572 | 573 | PSM 2 |
| 514 | 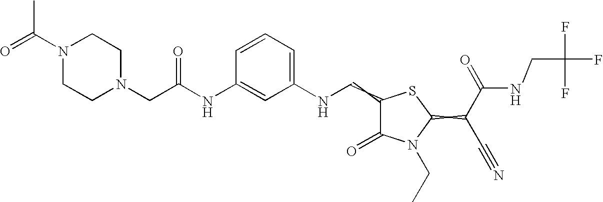 | 9.94 | 579 | 580 | PSM 2 |
| 515 | 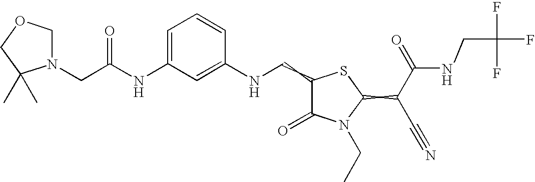 | 5.96 | 552 | 553 | PSM 2 |
| 516 | 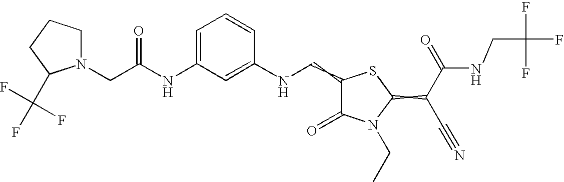 | 9.69 | 590 | 591 | PSM 2 |
| 517 | 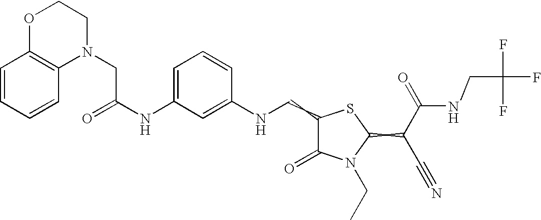 | 7.21 | 586 | 587 | PSM 2 |
| 518 | 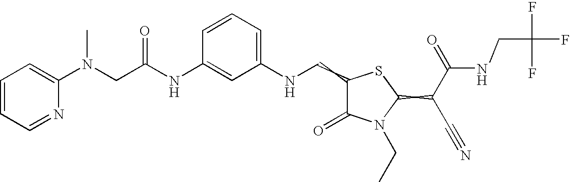 | 7.18 | 559 | 560 | PSM 2 |
| 519 | 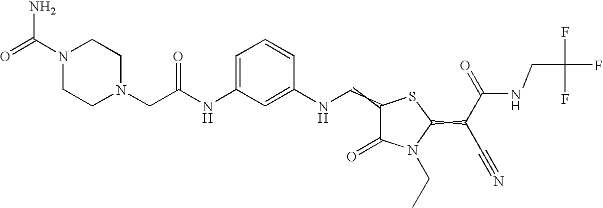 | 6.63 | 580 | 581 | PSM 2 |
| 520 | 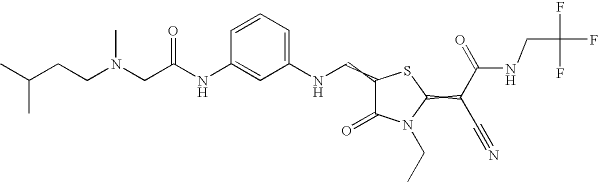 | 6.43 | 552 | 553 | PSM 2 |
| 521 | 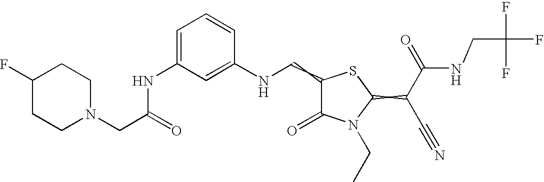 | 6.86 | 554 | 555 | PSM 2 |
| 522 | 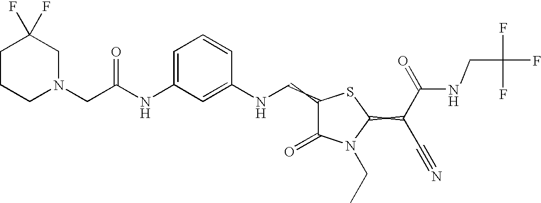 | 6.89 | 572 | 573 | PSM 2 |
| 523 | 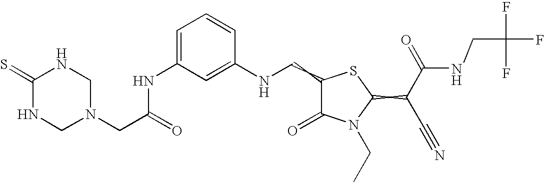 | 9.61 | 568 | 569 | PSM 2 |
| 524 | 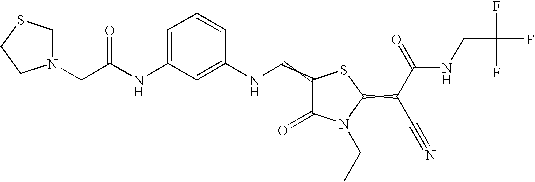 | 6.95 | 540 | 541 | PSM 2 |
| 525 | 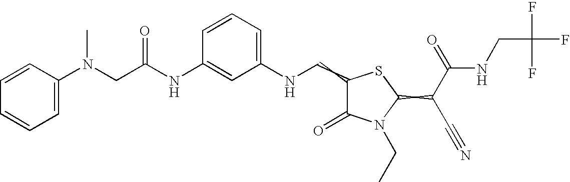 | 6.31 | 558 | 559 | PSM 2 |
| 526 | 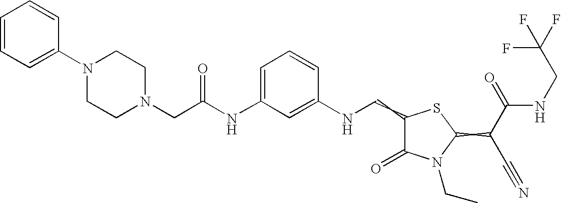 | 6.86 | 613 | 614 | PSM 2 |
| 527 | 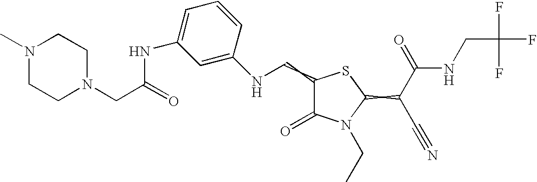 | 7.33 | 551 | 552 | PSM 2 |
| 528 | 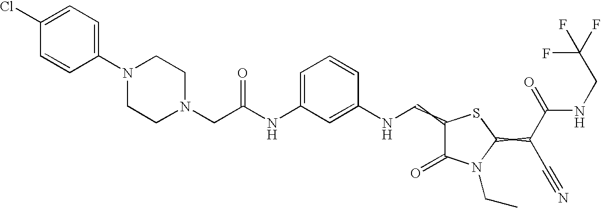 | 6.48 | 647 | 648 | PSM 2 |
| 529 | 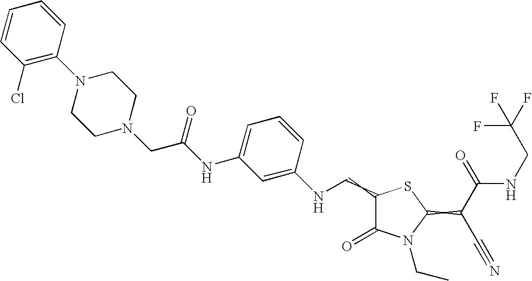 | 7.89 | 647 | 648 | PSM 2 |
| 530 | 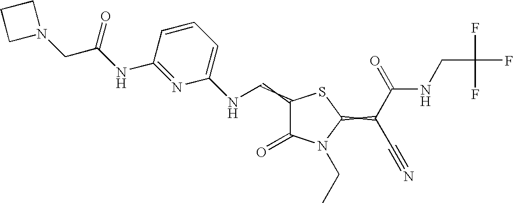 | 0.0 | 509 | 510 | PSM2 |
| 531 | 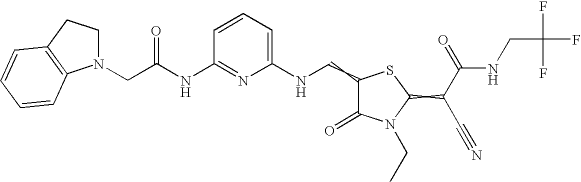 | 10.48 | 571 | 572 | PSM 2 |
| 532 | 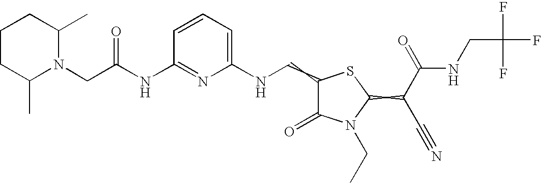 | 7.47 | 565 | 566 | PSM 2 |
| 533 | 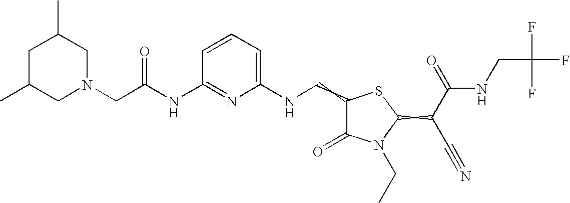 | 7.74 | 565 | 566 | PSM 2 |
| 534 | 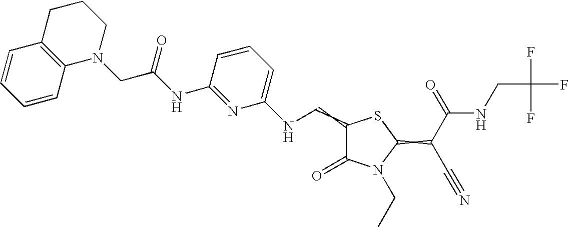 | 10.63 | 585 | 586 | PSM 2 |
| 535 | 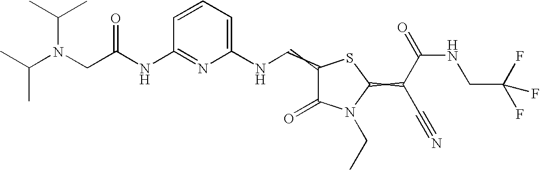 | 7.21 | 553 | 554 | PSM 2 |
| 536 | 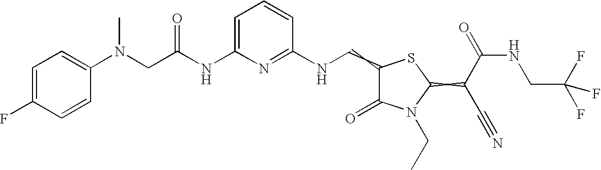 | 10.44 | 577 | 578 | PSM 2 |
| 537 | 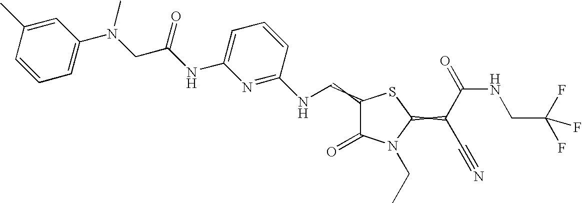 | 10.86 | 573 | 574 | PSM 2 |
| 538 | 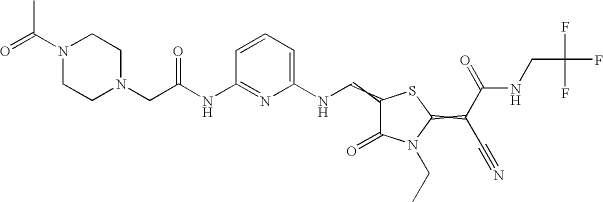 | 6.5 | 580 | 581 | PSM 2 |
| 539 | 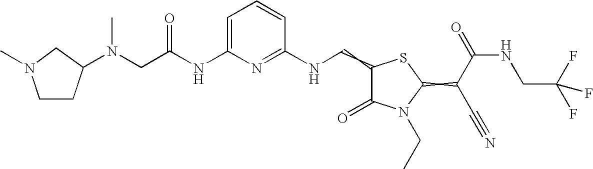 | 6.14 | 566 | 567 | PSM 2 |
| 540 | 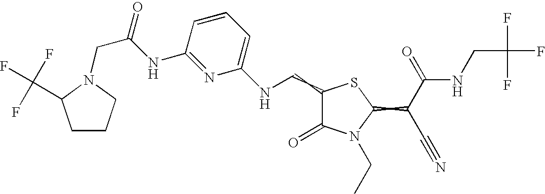 | 10.05 | 591 | 592 | PSM 2 |
| 541 | 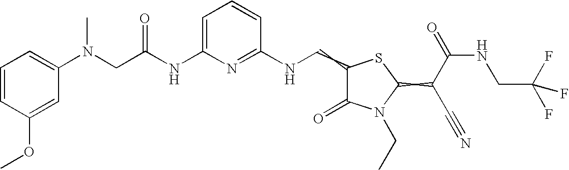 | 10.31 | 589 | 590 | PSM 2 |
| 542 | 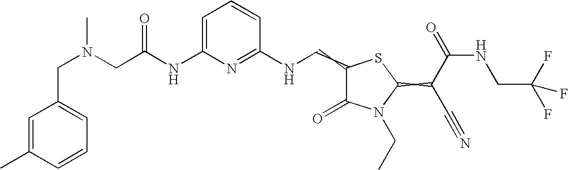 | 7.79 | 587 | 588 | PSM 2 |
| 543 | 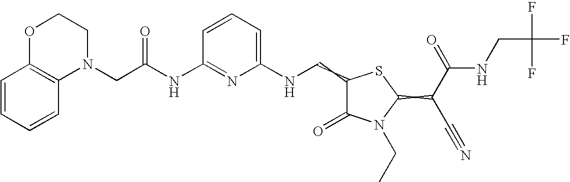 | 10.04 | 587 | 588 | PSM 2 |
| 544 | 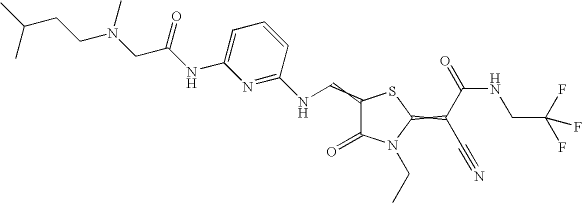 | 7.61 | 553 | 554 | PSM 2 |
| 545 | 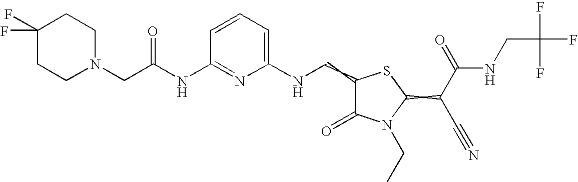 | 7.18 | 573 | 574 | PSM 2 |
| 546 | 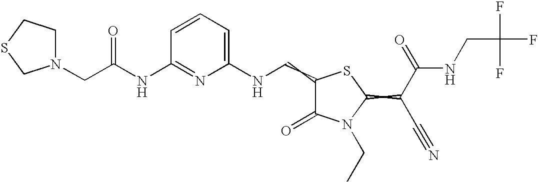 | 7.26 | 541 | 542 | PSM 2 |
| 547 | 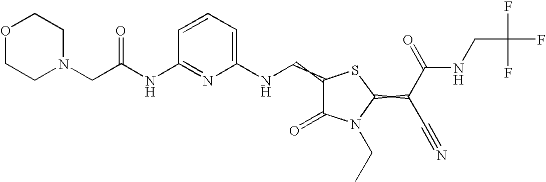 | 6.74 | 539 | 540 | PSM 2 |
| 548 | 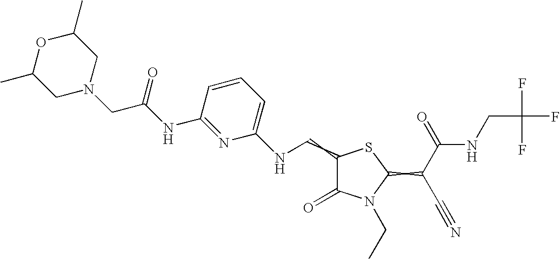 | 7.06 | 567 | 568 | PSM 2 |
| 549 | 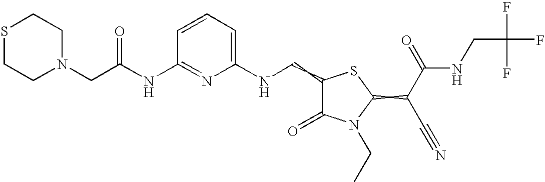 | 6.93 | 555 | 556 | PSM 2 |
| 550 | 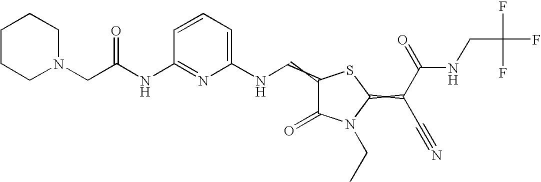 | 7.01 | 537 | 538 | PSM 2 |
| 551 | 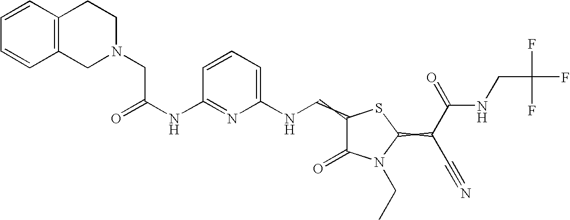 | 7.61 | 585 | 586 | PSM 2 |
| 552 | 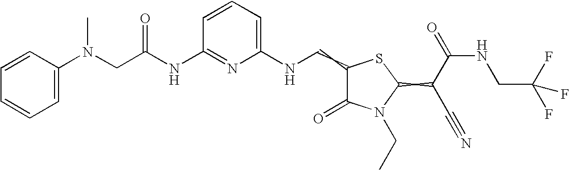 | 9.95 | 559 | 560 | PSM 2 |
| 553 | 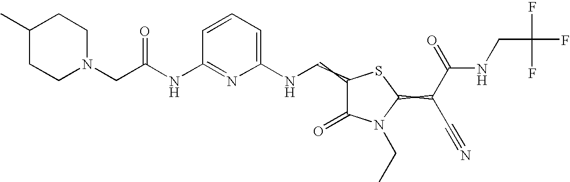 | 7.23 | 551 | 552 | PSM 2 |
| 554 | 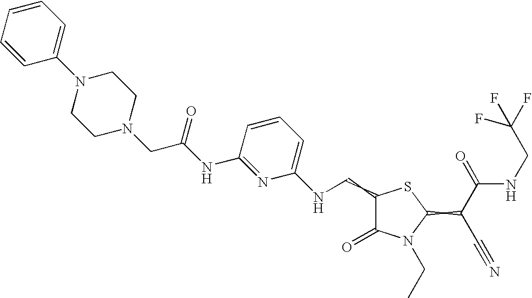 | 0.0 | 614 | 615 | PSM 2 |
| 555 | 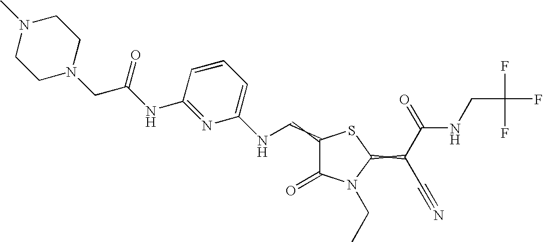 | 6.54 | 552 | 553 | PSM 2 |
| 556 | 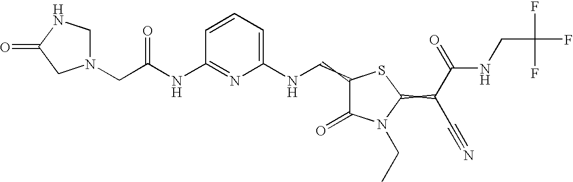 | 6.67 | 538 | 539 | PSM 2 |
| 557 | 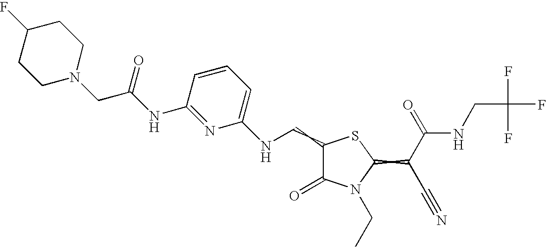 | 7.09 | 555 | 556 | PSM 2 |
| 558 | 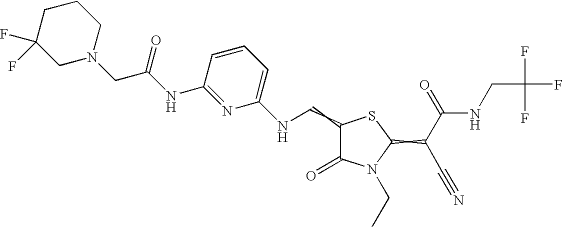 | 7.48 | 573 | 574 | PSM 2 |
| 559 | 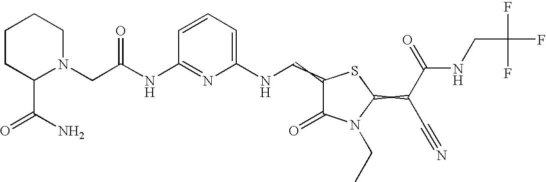 | 6.65 | 580 | 581 | PSM 2 |
| 560 | 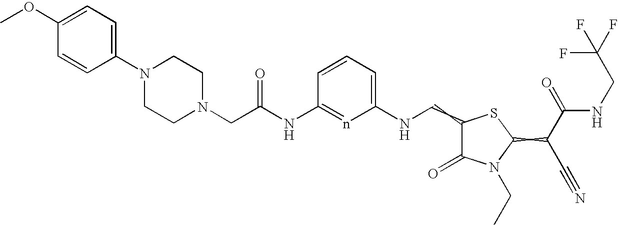 | 7.76 | 644 | 645 | PSM 2 |
| 561 | 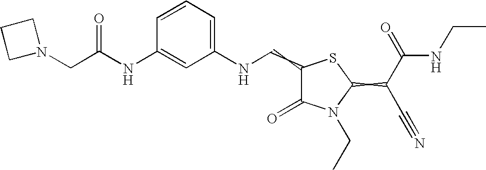 | 6.28 | 455 | 456 | PSM 2 |
| 562 | 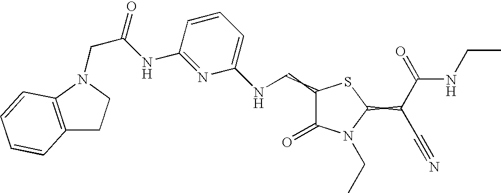 | 10.01 | 517 | 518 | PSM 2 |
| 563 | 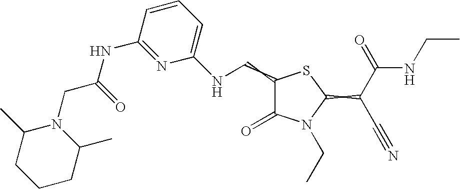 | 3.37 | 511 | 512 | PSM 2 |
| 564 | 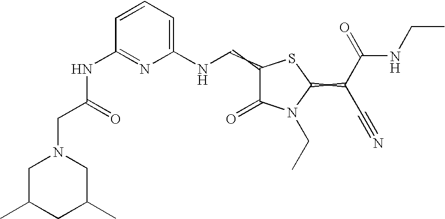 | 7.24 | 511 | 512 | PSM 2 |
| 565 | 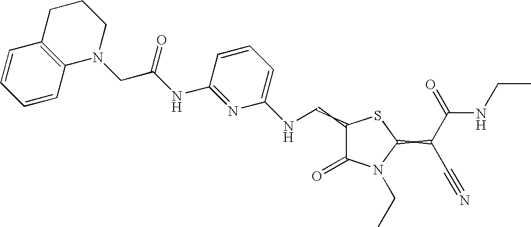 | 10.38 | 531 | 532 | PSM 2 |
| 566 | 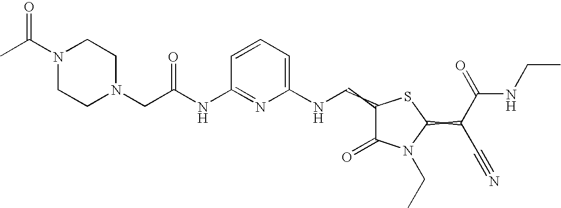 | 6.01 | 526 | 527 | PSM 2 |
| 567 | 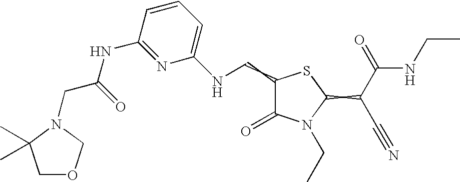 | 6.32 | 499 | 500 | PSM 2 |
| 568 | 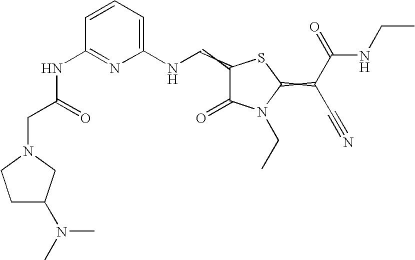 | 5.68 | 512 | 513 | PSM 2 |
| 569 | 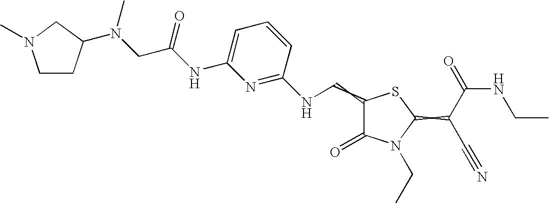 | 5.66 | 512 | 513 | PSM 2 |
| 570 | 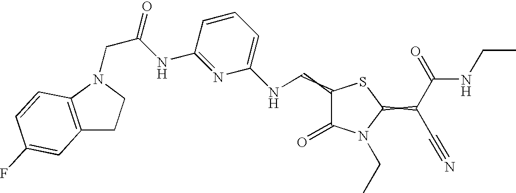 | 9.96 | 535 | 536 | PSM 2 |
| 571 | 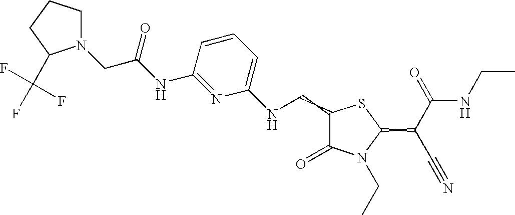 | 9.92 | 537 | 538 | PSM 2 |
| 572 | 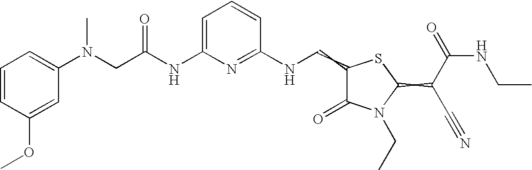 | 9.58 | 535 | 536 | PSM 2 |
| 573 | 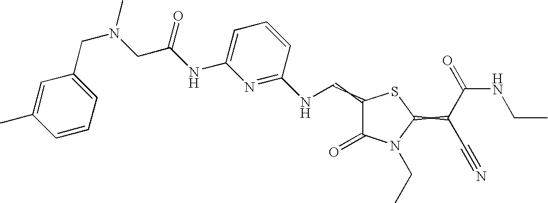 | 7.36 | 533 | 534 | PSM 2 |
| 574 | 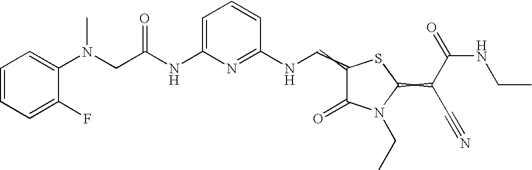 | 10.06 | 523 | 524 | PSM 2 |
| 575 | 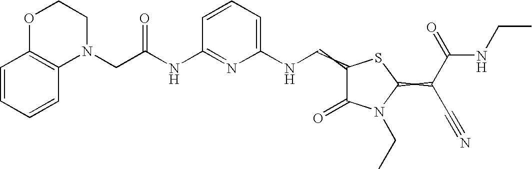 | 7.04 | 529 | 530 | PSM 2 |
| 576 | 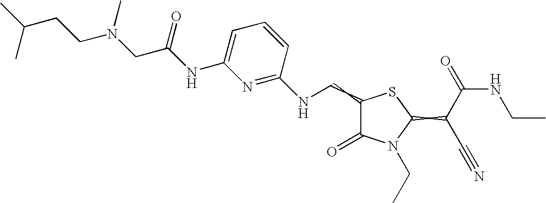 | 9.64 | 533 | 534 | PSM 2 |
| 577 | 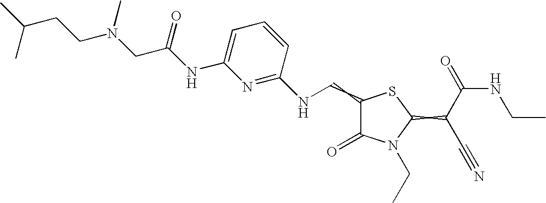 | 7.22 | 499 | 500 | PSM 2 |
| 578 | 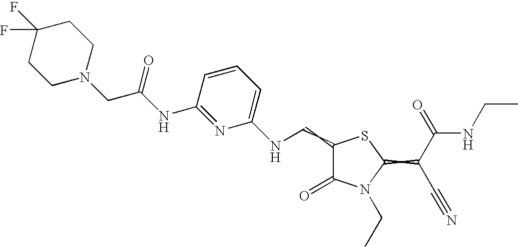 | 6.88 | 519 | 520 | PSM 2 |
| 579 | 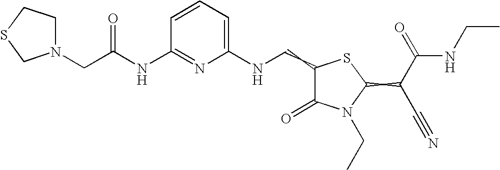 | 6.71 | 487 | 488 | PSM 2 |
| 580 | 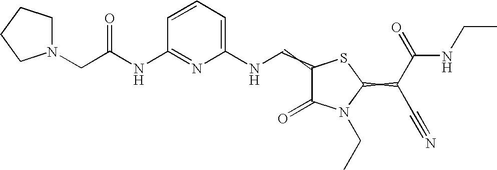 | 6.33 | 469 | 470 | PSM 2 |
| 581 | 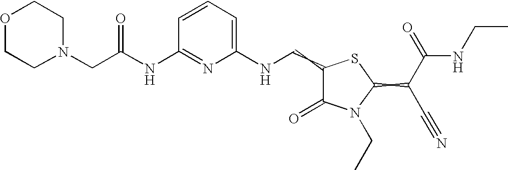 | 6.22 | 485 | 486 | PSM 2 |
| 582 | 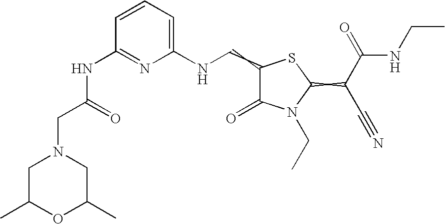 | 6.69 | 513 | 514 | PSM 2 |
| 583 | 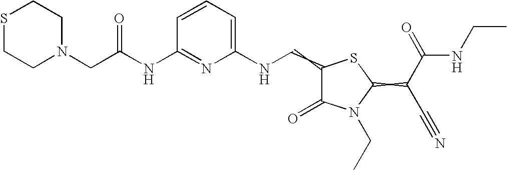 | 6.56 | 501 | 502 | PSM 2 |
| 584 | 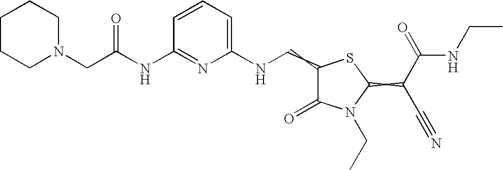 | 6.48 | 483 | 484 | PSM 2 |
| 585 | 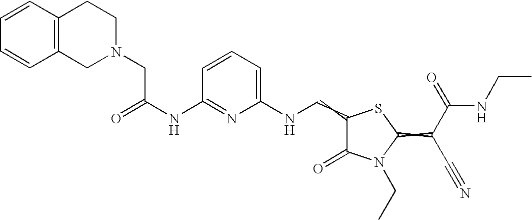 | 7.19 | 531 | 532 | PSM 2 |
| 586 | 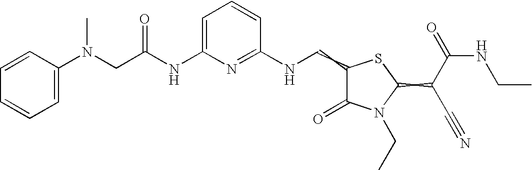 | 9.81 | 505 | 506 | PSM 2 |
| 587 | 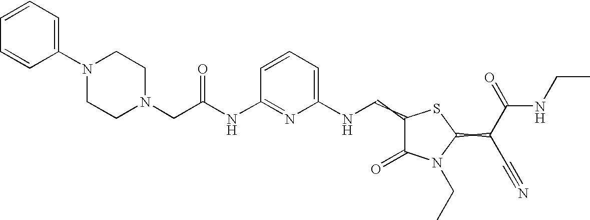 | 7.33 | 560 | 561 | PSM 2 |
| 588 | 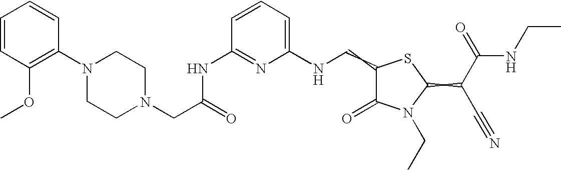 | 7.41 | 590 | 591 | PSM 2 |
| 589 | 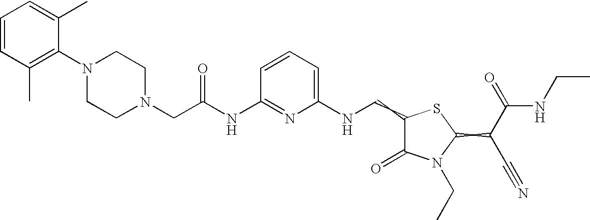 | 8.03 | 588 | 589 | PSM 2 |
| 590 | 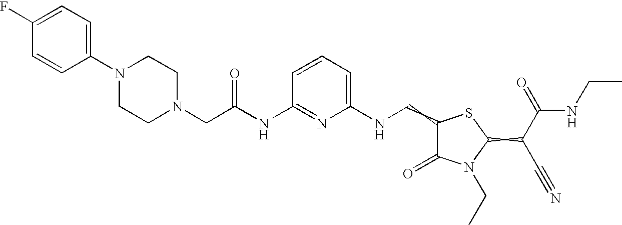 | 7.43 | 578 | 579 | PSM 2 |
| 591 | 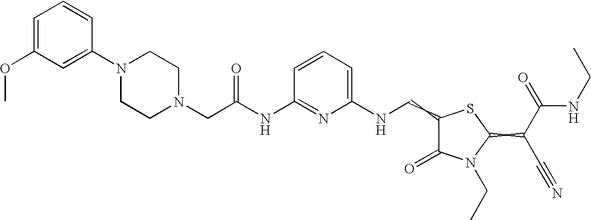 | 7.46 | 590 | 591 | PSM 2 |
| 592 | 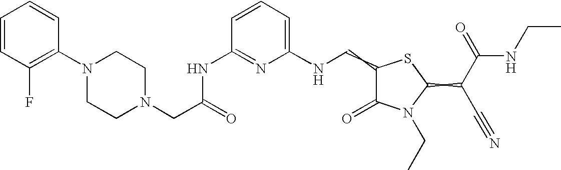 | 7.57 | 578 | 579 | PSM 2 |
| 593 | 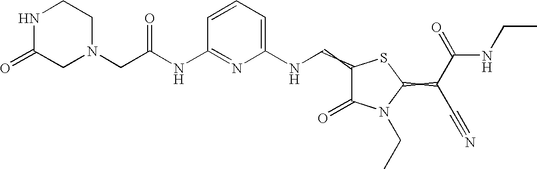 | 6.0 | 498 | 499 | PSM 2 |
| 594 | 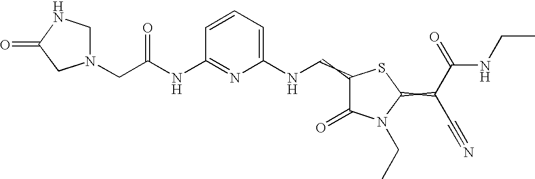 | 6.1 | 484 | 485 | PSM2 |
| 595 | 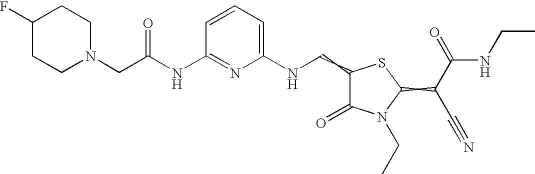 | 6.57 | 501 | 502 | PSM 2 |
| 596 | 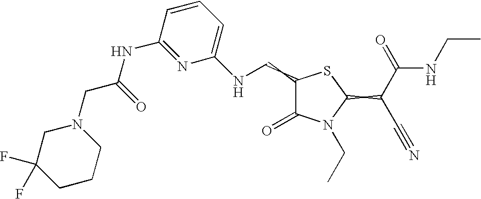 | 7.07 | 519 | 520 | PSM 2 |
| 597 | 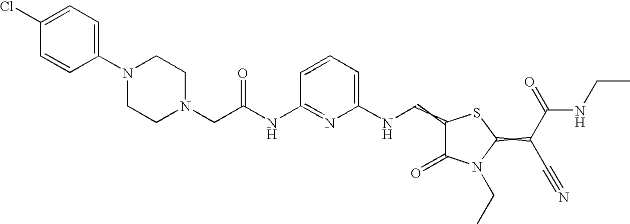 | 7.8 | 594 | 595 | PSM 2 |
| 598 | 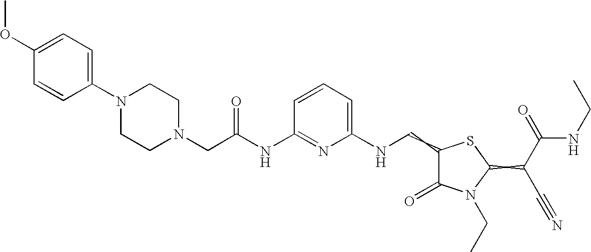 | 7.26 | 590 | 591 | PSM 2 |

### 6. Other Amides

In analogous fashion the following compounds can be created:

TABLE
Amides (2)

| Example | Structure |
|---|---|
| 599 | 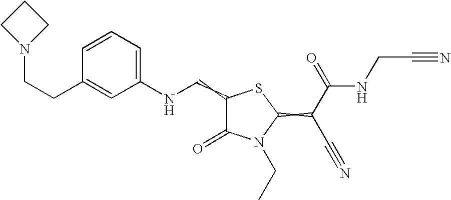 |
| 600 | 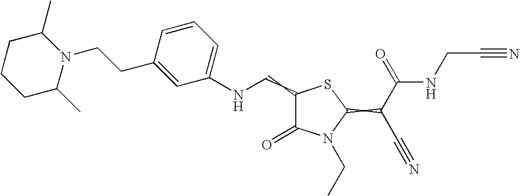 |
| 601 | 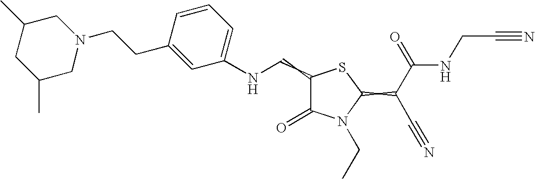 |
| 602 | 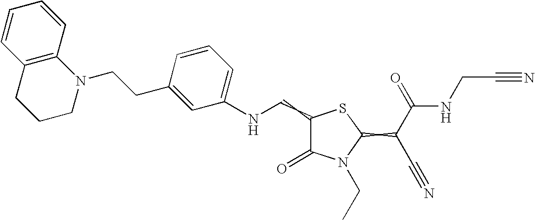 |
| 603 | 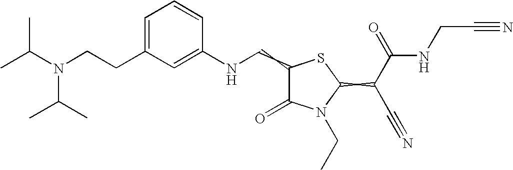 |
| 604 | 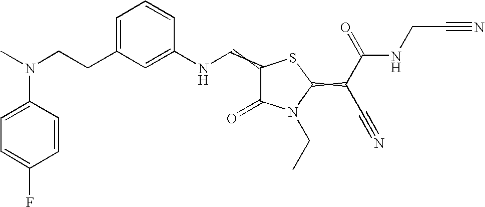 |
| 605 | 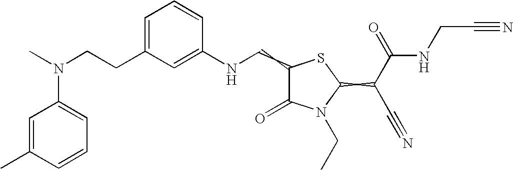 |
| 606 | 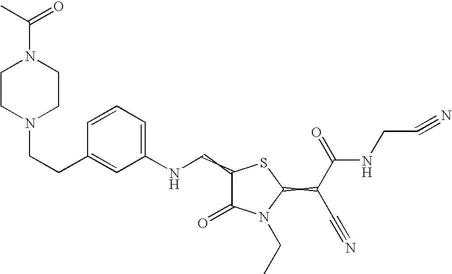 |
| 607 | 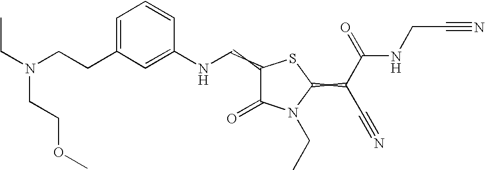 |
| 608 | 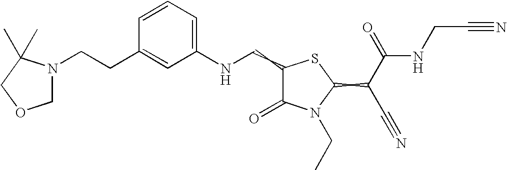 |
| 609 | 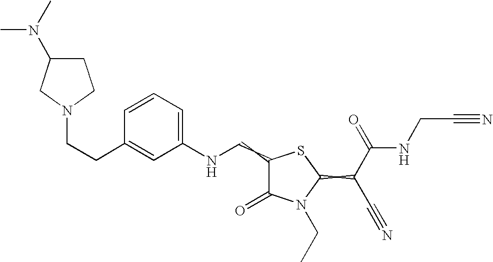 |
| 610 | 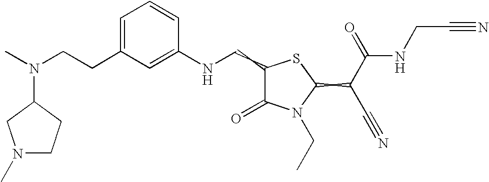 |
| 611 | 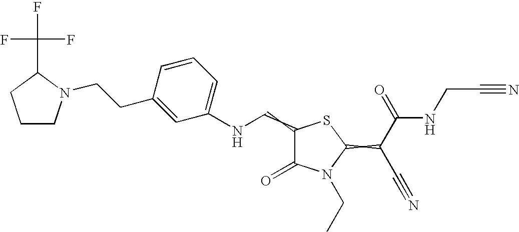 |
| 612 | 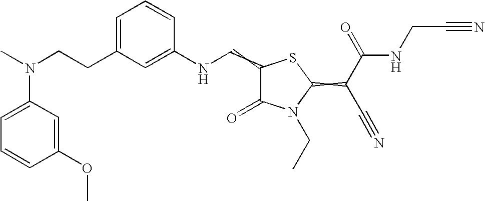 |
| 613 | 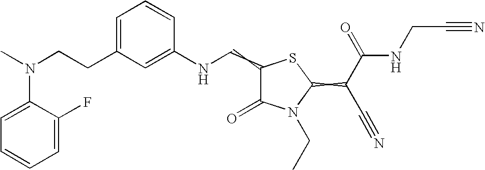 |
| 614 | 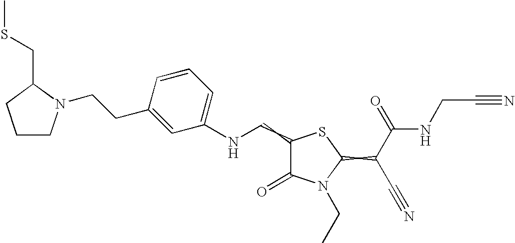 |
| 615 | 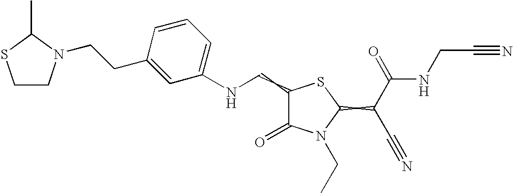 |
| 616 | 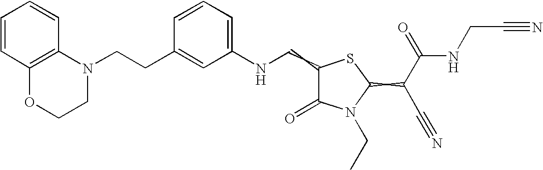 |
| 617 | 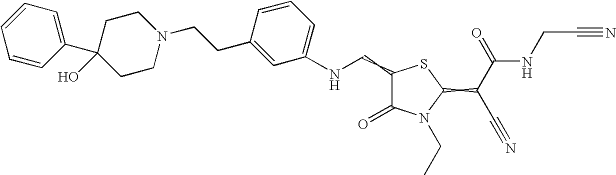 |
| 618 | 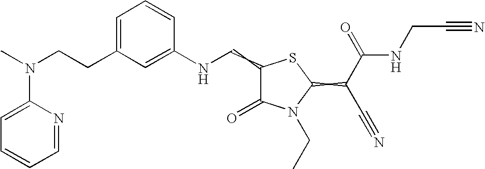 |
| 619 | 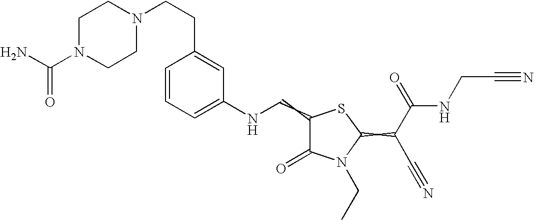 |
| 620 | 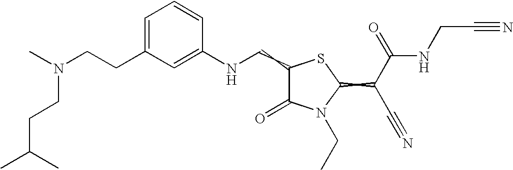 |
| 621 | 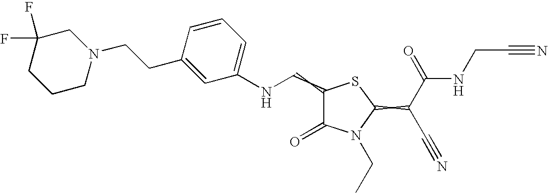 |
| 622 | 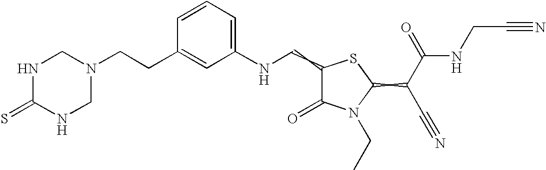 |
| 623 | 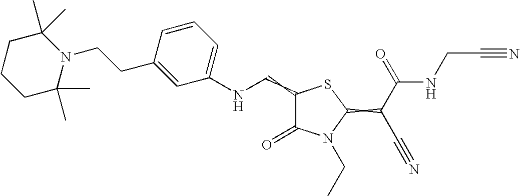 |
| 624 | 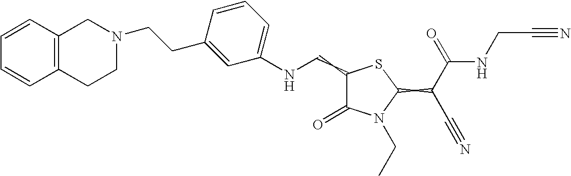 |
| 625 | 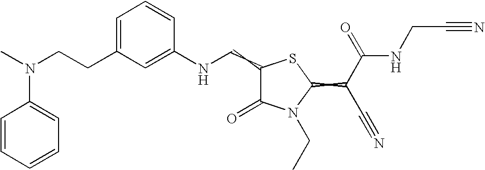 |
| 626 | 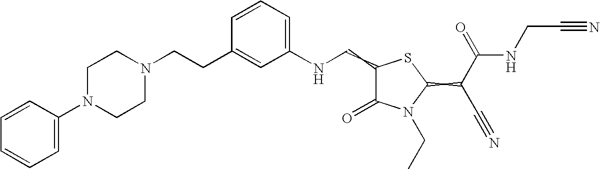 |
| 627 | 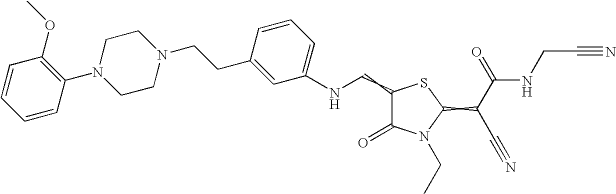 |
| 628 | 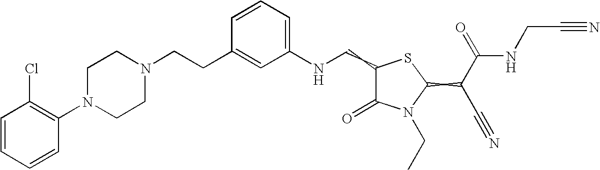 |
| 629 | 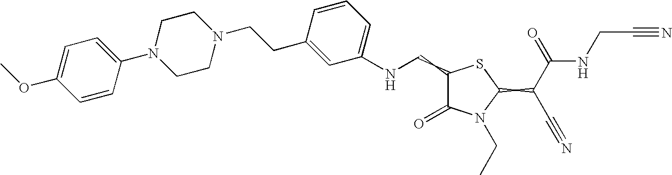 |
| 630 | 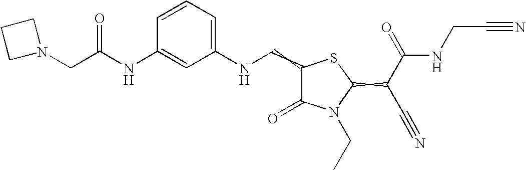 |
| 631 | 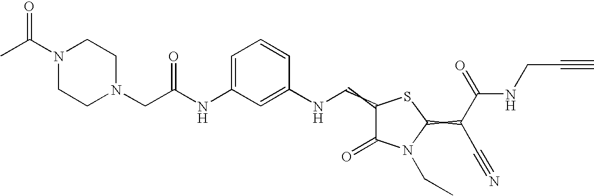 |
| 632 | 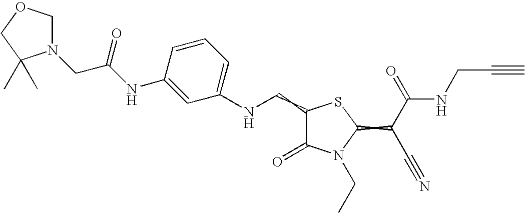 |
| 633 | 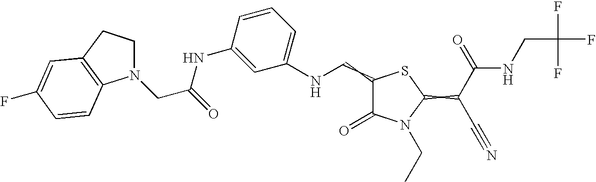 |
| 634 | 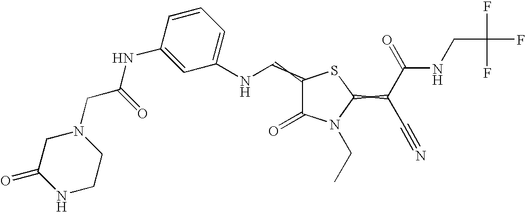 |
| 635 | 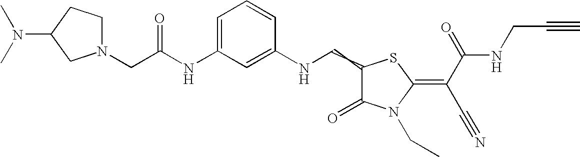 |
| 636 | 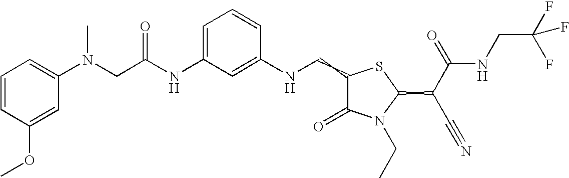 |
| 637 | 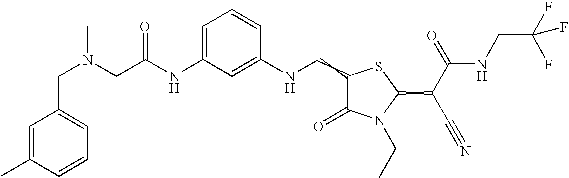 |
| 638 | 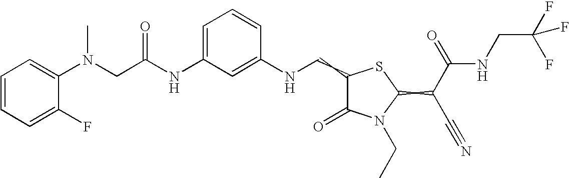 |
| 639 | 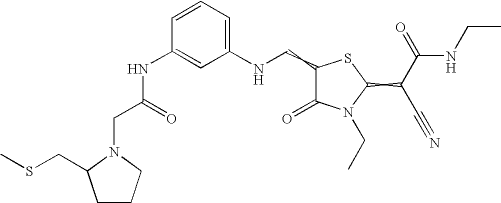 |
| 640 | 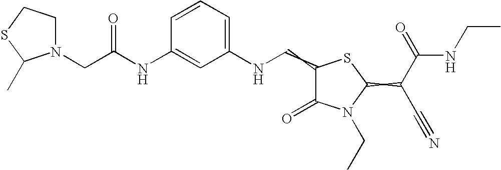 |
| 641 | 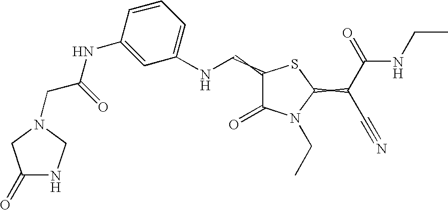 |
| 642 | 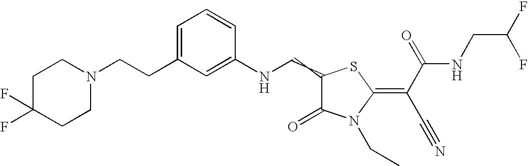 |
| 643 | 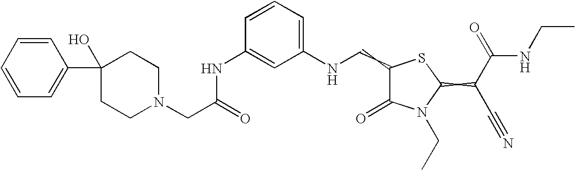 |
| 644 | 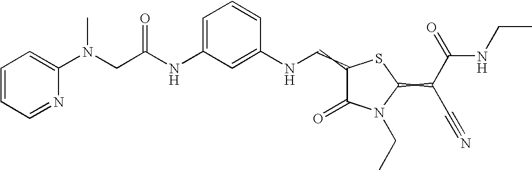 |
| 645 | 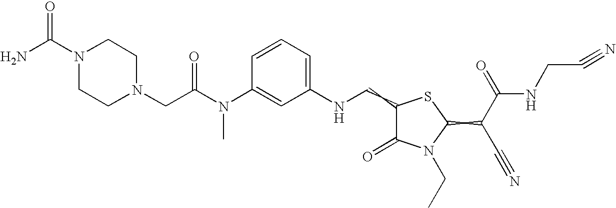 |
| 646 | 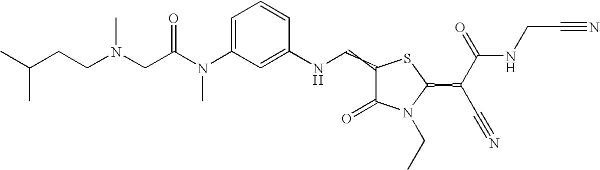 |
| 647 | 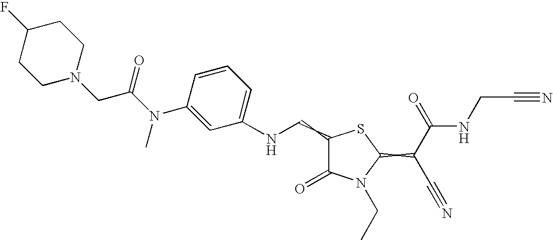 |
| 648 | 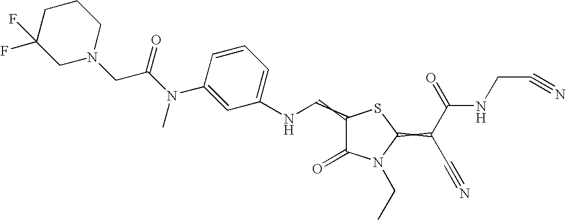 |
| 649 | 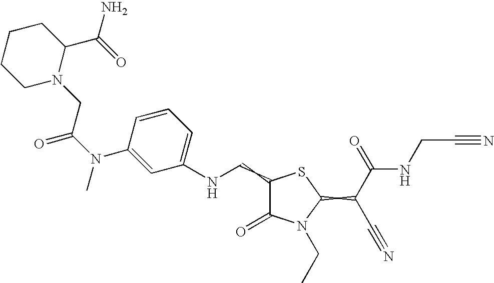 |
| 650 | 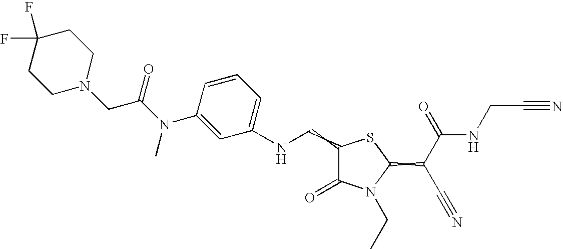 |
| 651 | 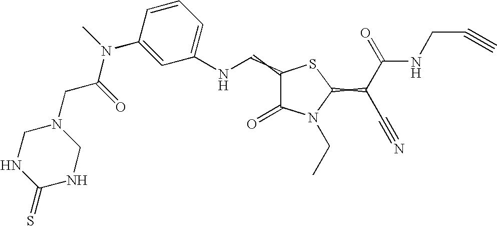 |
| 652 | 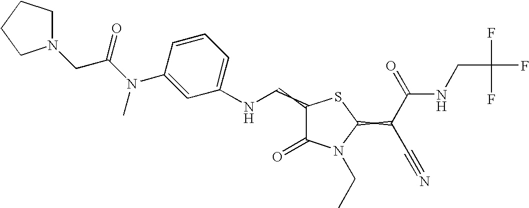 |
| 653 | 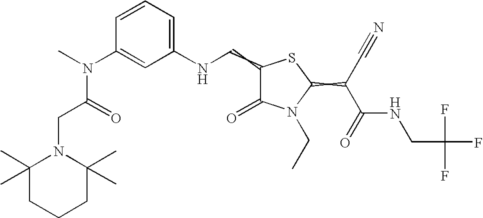 |
| 654 | 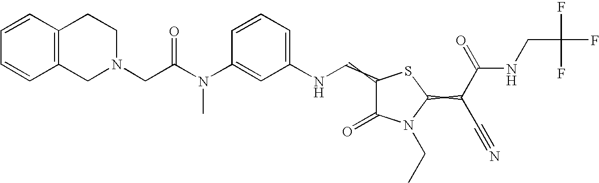 |
| 655 | 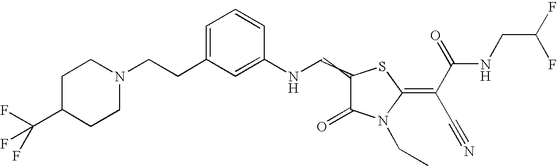 |
| 656 | 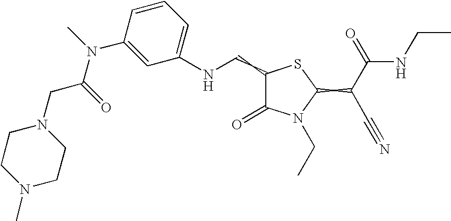 |
| 657 | 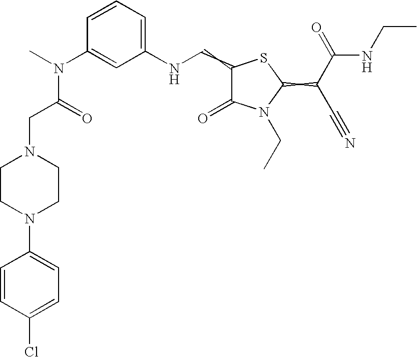 |
| 658 | 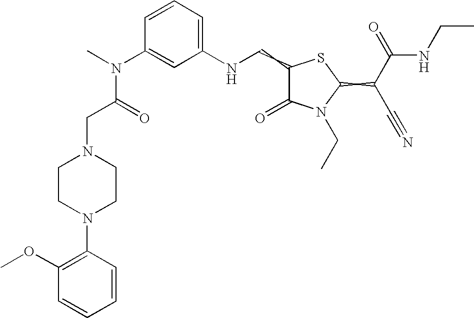 |
| 659 | 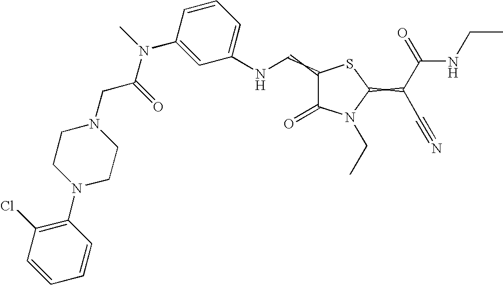 |
| 660 | 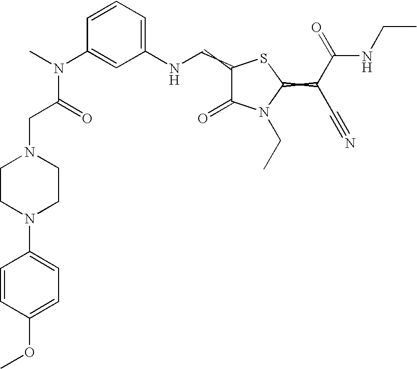 |
| 661 | 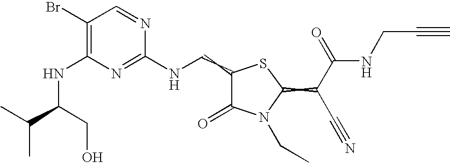 |
| | 2-[5-[1-[5-Bromo-4-((R)-1-hydroxymethyl-2-methyl- |
| | propylamino)-pyrimidin-2-ylamino]-meth-(E/Z)- |
| | ylidene]-3-ethyl-4-oxo-thiazolidin-(2-(E oder Z))- |
| | ylidene]-2-cyano-N-prop-2-ynyl-acetamide |
| 662 | 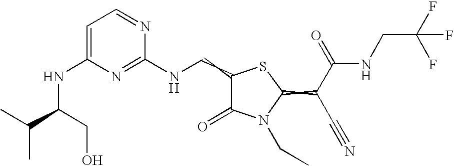 |
| | 2-Cyano-2-[3-ethyl-5-[1-[4-((R)-1-hydroxymethyl-2- |
| | methyl-propylamino)-pyrimidin-2-ylamino]-meth- |
| | (E/Z)-ylidene]-4-oxo-thiazolidine-(2-(E oder Z))- |
| | ylidene]-N-(2,2,2-trifluoro-ethyl)-acetamide |
| 663 | 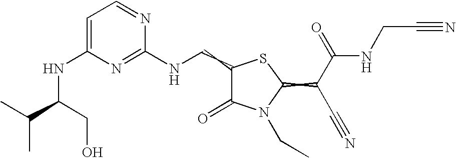 |
| | 2-Cyano-N-cyanomethyl-2-[3-ethyl-5-[1-[4-((R)-1- |
| | hydroxymethyl-2-methyl-propylamino)-pyrimidin-2- |
| | ylamino]-meth-(E/Z)-ylidene]-4-oxo-thiazolidine-(2- |
| | (E oder Z))-ylidene]-acetamide |
| 664 | 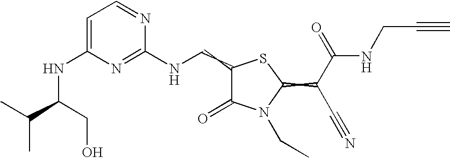 |
| | 2-Cyano-2-[3-ethyl-5-[1-[4-((R)-1-hydroxymethyl-2- |
| | methyl-propylamino)-pyrimidin-2-ylamino]-meth- |
| | (E/Z)-ylidene]-4-oxo-thiazolidin-(2-(E oder Z))- |
| | ylidene]-N-prop-2-ynyl-acetamide |
| 665 | 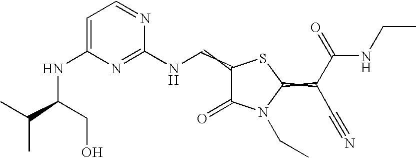 |
| | 2-Cyano-N-ethyl-2-[3-ethyl-5-[1-[4-((R)-1- |
| | hydroxymethyl-2-methyl-propylamino)-pyrimidin-2- |
| | ylamino]-meth-(E/Z)-ylidene]-4-oxo-thiazolidin-(2-(E |
| | oder Z))-ylidene]-acetamide |
| 666 | 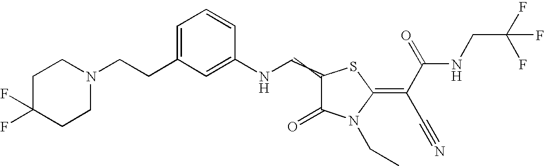 |
| 667 | 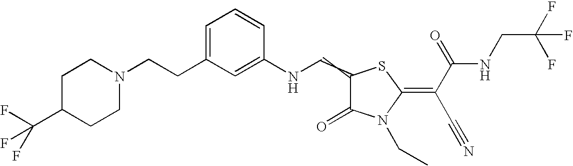 |
| 668 | 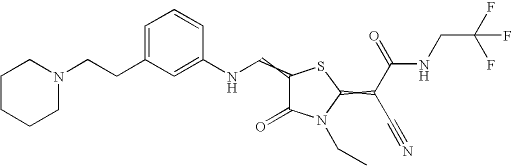 |
| 669 | 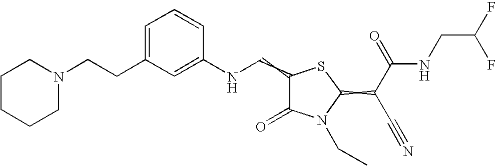 |
| 670 | 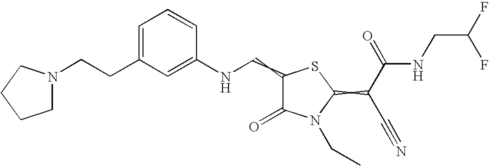 |

### Examples

The following examples describe the biological effects of the invented compounds:

### PLK Enzyme-Assay

Recombinant human Plk-1 (6×His) was purified from insect cells (Hi5) infected with a baculovirus.

10 ng (recombinant created, purified) PLK enzyme is incubated for 90 minutes at room temperature with biotin casein and 33P-γ-ATP as a sub-stratum in a volume of 15 μl in 384 well Greiner Small Volume Microtiterplates (final concentration in buffer: 660 ng/ml PLK; 0.7 μM Casein, 0.5 μM ATP incl. 400 nCi/ml 33P-γ-ATP; 10 mM MgCl2, 1 mM MnCl2; 0.01% NP40; 1 mM DTT, Protease inhibitors; 0.1 mM Na2VO3 in 50 mM HEPES pH 7.5). At the end of the reaction a 5 μl stop solution (500 μM ATP; 500 mM EDTA; 1% triton X100; 100 mg/ml streptavidin coated SPA Beads in PBS) was added. After the microtiter plate is closed with foil, the beads are sedimented through centrifugation (10 min., 1500 rpm). The fixing of the 33P-γ-ATP in casein was set as a measure of the enzyme activity by β-Counting. The measure of inhibitor activity was referenced against a control solution (=unrestricted enzyme activity=0% inhibition) and the average value of several deposits that contained 300 μM Wortmannin (=fully restricted enzyme activity=100% inhibition).

Test substances were introduced in various concentrations (0 μM, and in the range of 0.01-30 μM). The final concentration of the dimethylsulfoxide solvent in all concentrations amounted to 1.5%.

### Proliferation Assay

Cultivated human MaTu breast tumor cells were spread on plates to a thickness of 5000 cells per measuring point in a 96-hole multititerplate in 200 μl of the appropriate growth medium. After 24 hours the cells of one plate (the zero-point plate) were stained with crystal violet (see below), while the medium of the other plates was replaced by fresh culture medium (200 μl), to which was added the test substances in various concentrations (0 μM and in the range of 0.01 to 30 μm; the final concentration of the dimethylsulfoxide solvent was 0.5%). The cells were incubated for 4 days in the presence of the test substances. The cell proliferation was determined by the staining of the cells with crystal violet. The cells were fixed by the addition of 20 μl per measuring point of an 11% glutaraldehyde solution for 15 minutes at room temperature. After the fixed cells were washed three times with water, the plates were dried at room temperature. The cells were stained by the addition of 100 μl per measuring point of a 0.1% crystal violet solution (pH set at pH3 through the addition of acetic acid). After the fixed cells were washed three times with water, the plates were dried at room temperature. The coloring was dissolved by the addition of 100 μl per measuring point of a 10% acetic acid solution. Extinction was determined photometrically at a wave length of 595 nm. The percentage change of the cell growth was calculated by normalizing the measured values at the extinction value of the zero-point plate (=0%) and the extinction of the untreated cells (0 μM) (=100%).

The results of the PLK-1 enzyme assay and of the proliferation assay are shown in the following table 1.

| | | | Inhibition of |
|---|---|---|---|
| | | | tumor cell |
| | | Inhibition | proliferation |
| Example | | PLK-1 | (MaTu) |
| no. | Structure | IC50 [μM] | IC50 [μM] |
| 55 | 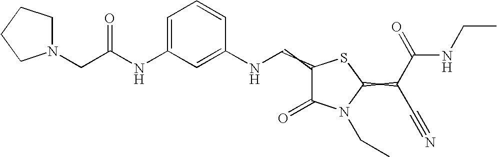 | 150 | 0.78 |
| 30 | 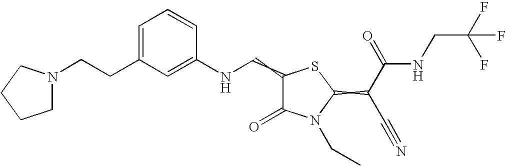 | 16 | 0.2 |
| 127 | 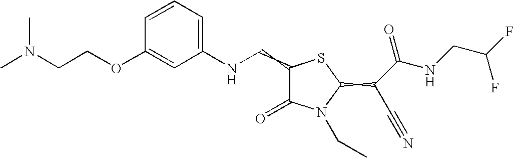 | 24 | 0.33 |
| 126 | 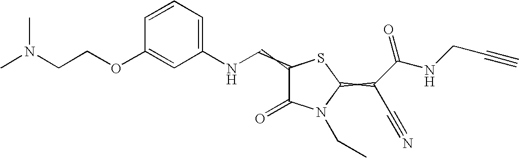 | 22 | 0.59 |
| 41 | 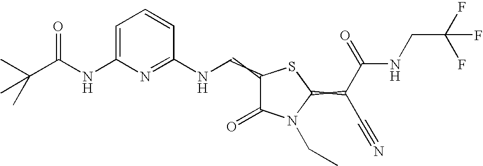 | 20 | 0.83 |
| 63 | 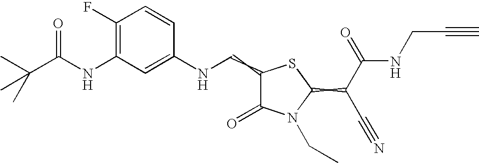 | 100 | 0.65 |

TABLE 2
Comparison with Current State of Technology

| | | | Inhibition of |
|---|---|---|---|
| | | | tumor cell |
| | | Inhibition | proliferation |
| Example | | PLK-1 | (MaTu) |
| no. | Structure | IC50 [μM] | IC50 [μM] |
| 30 | 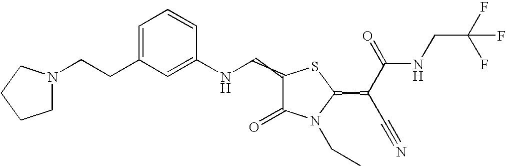 | 16 | 0.2 |
| Comparison 527 from PCT/EP20 04/012242 | 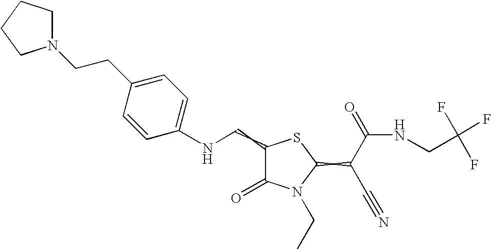 | 100 | 2.8 |
| 127 | 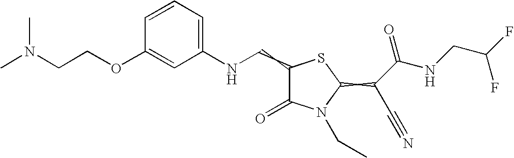 | 24 | 0.33 |
| Comparison 310 from PCT/EP20 04/012242 | 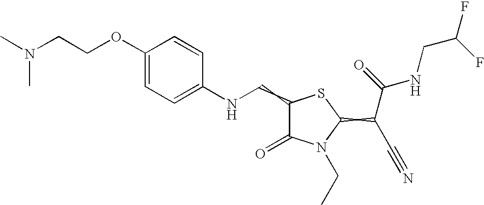 | 74 | 5.6 |
| 126 | 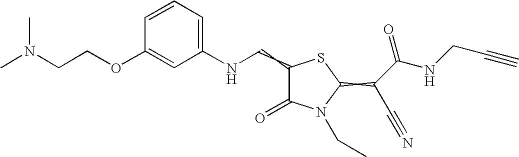 | 22 | 0.59 |
| Comparison 307 from PCT/EP20 04/012242 | 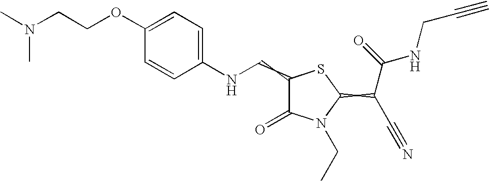 | 71 | 1.7 |

TABLE 3
Comparison with Current State of Technology

| | | | Inhibition of |
|---|---|---|---|
| | | | tumor cell |
| | | Inhibition | proliferation |
| Example | | PLK-1 | (MaTu) |
| no. | Structure | IC50 [μM] | IC50 [μM] |
| 41 | 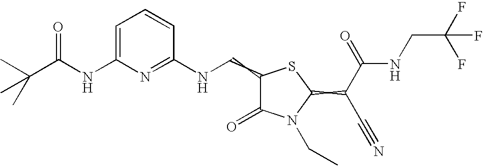 | 20 | 0.83 |
| Comparison 326 from PCT/EP20 04/012242 | 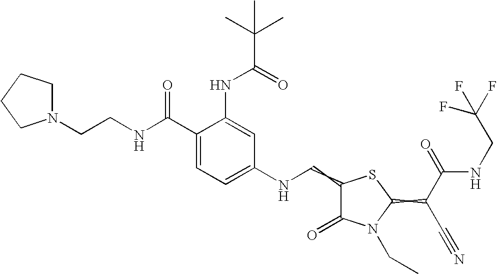 | 73 | 1.6 |
| 63 | 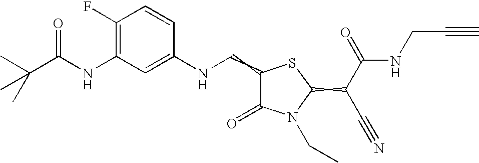 | 100 | 0.65 |
| Comparison 323 from PCT/EP20 04/012242 | 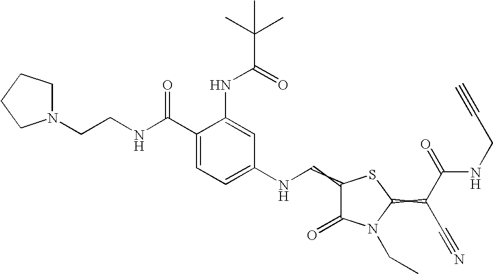 | 140 | 2.5 |

From Table 1 it can be seen that the present invented compounds of general formula I inhibit PLK. Furthermore the expert can see from Tables 2 and 3 that the present invented substances are also better than the current state of technology.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 102004061503.9, filed Dec. 15, 2004 and U.S. Provisional Application Ser. No. 60/637,777, filed Dec. 22, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### BRIEF DESCRIPTION OF THE DRAWINGS

FIG. 1 represents schematically cell cycle activation and separation and the role of Plk1 in those processes.

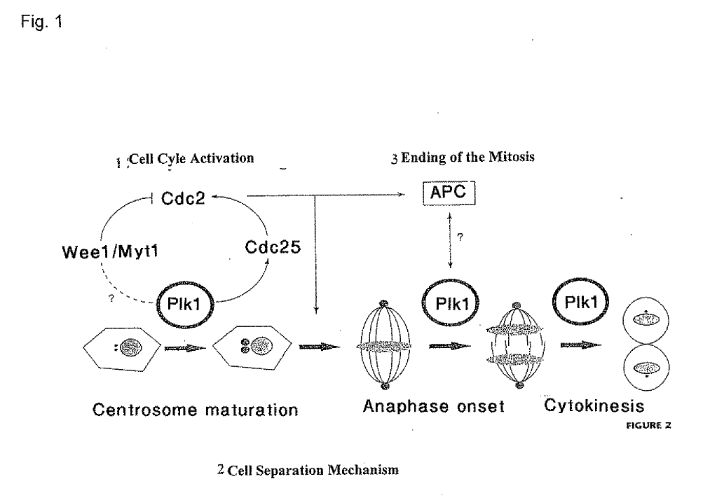

1. Compounds of general formula I 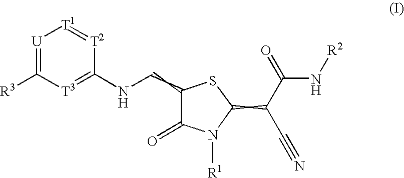
in which the meaning is as follows:
T¹, T² and T³ stand independently of one another for —CH═ or —N═ and T² can also stand for (—CF)═,
U stands for —CR⁴═ or —N═,
R¹ stands for C₁-C₃-alkyl or cyclopropyl substituted once or several times, the same or different, with halogen,
R¹ stands for C₁-C₃-alkyl, C₃-C₄-alkenyl, C₃-C₄-alkinyl or cyclopropyl or methyl substituted hydroxyethyl substituted as needed once or several times, the same or different, with cyano, cyclopropyl, ethinyl or halogen,
R³ stands for K, L or M or for R¹⁵,
K stands for C₁-C₃-Alkyl or C₂-C₄-Alkenyl substituted once or several times, the same or different, with X,
X stands for halogen, hydroxy or for the —OR⁶, —NR¹⁰R¹¹ group or for C₂-C₁₀-heterocycloalkyl, in which the heterocycloalkyl in the ring contains at least one atom, the same or different, from the following group of nitrogen, oxygen, or sulfur and may also be made up of one or more —(CO)—, —(C═S)— or —SO₂— groups in the ring, and may be contained in one or more double bonds in the ring, and the ring itself may be substituted C₁-C₃-alkyl substituted once or several times, the same or different, substituted with cyano, halogen, hydroxy, aryl or with the —(CO)—R⁵, —NR¹²R¹³ group or substituted once or several times, the same or different, with halogen, hydroxy or C₁-C₃-alkylthio, in which the aryl itself may be substituted once or several times, the same or different, with cyano, halogen or C₁-C₃-alkoxy,
L stands for the group —O—R⁷, —O—(CH₂)ₙ—(CO)—NH—R⁸, —O—(CH₂)ₙ—(CO)—R¹⁵ or —O—(CH₂)ₙ—(CO)—O—R⁸,
M stands for the group —NH—R⁹, —NH—(CO)—OH, —NH—(CO)—O—R⁹ or —NR¹²—(CO)—R¹⁶,
R⁴ stands for hydrogen, cyano or halogen or for methyl substituted once or several times, the same or different, with halogen,
R⁵ stands for C₁-C₄-alkyl, phenyl, or —NR¹²R¹³,
R⁶ stands for —SO₂—R¹⁴,
R⁷ stands for C₁-C₃-alkyl substituted once or several times, the same or different, with —NR¹²R¹³ or C₂-C₁₀-heterocycloalkyl, in which the heterocycloalkyl in the ring contains at least one atom, equally or variously, of the following group of nitrogen, oxygen, or sulfur and may be made up of one or more —(CO)— or —SO₂— groups in the ring and may contain one or more double bonds in the ring, and the ring itself may be substituted C₁-C₃-alkyl substituted once or several times, the same or different, with halogen, aryl or substituted once or several times, the same or different, with halogen,
R⁸ stands for C₁-C₃-Alkyl, C₃-C₄-alkenyl or C₃-C₄-alkinyl substituted once or several times, the same or different, with cyano, cyclopropyl or halogen,
R⁹ stands for C₁-C₅-alkyl, C₂-C₄-alkenyl, cyclopropyl or C₂-C₁₀-heterocycloalkyl substituted once or several times, the same or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹⁴ group, in which the heterocycloalkyl in the ring contains at least one atom the same or different, of the following group of nitrogen, oxygen, or sulfur and may be made up of one or more —(CO)— or —SO₂— groups in the ring and may contain one or more double bonds in the ring, and the ring itself may be substituted once or several times, the same or different, with halogen, cyano, hydroxy, aryl or with the —(CO)—R⁵, —(CO)—O—R¹², —(SO₂)—R¹⁴, —NR¹²R¹³ group or may be substituted once or several times, the same or different, with halogen, hydroxy, C₁-C₃-alkylthio or phenyl substituted C₁-C₃-alkyl, in which the aryl itself may once or several times, the same or different substituted with halogen or C₁-C₃-alkoxy,
R¹⁰ and R¹¹ stand for independent of one another C₁-C₈-alkyl, C₂-C₁₀-heterocycloalkyl, aryl, —(CH₂)ₙ-aryl or heteroaryl, substituted once or several times, the same or different, with halogen, C₁-C₃-alkyl, C₁-C₃-alkoxy, substituted C₁-C₅-alkyl, C₂-C₁₀-heterocycloalkyl, aryl, —(CH₂)ₙ-aryl or heteroaryl, in which the heterocycloalkyl in the ring contains at least one atom the same or different, of the following group of nitrogen, oxygen, or sulfur and may be made up of one or more —(CO)— or —SO₂— groups in the ring and may contain one or more double bonds in the ring,
R¹² und R¹³ stand for hydrogen or C₁-C₄-alkyl independent of one another,
R¹⁴ stands for C₁-C₃-alkyl or for aryl
R¹⁵ stands for C₂-C₁₀-heterocycloalkyl substituted once or several times, the same or different, with C₁-C₃-alkyl or —(CH₂)ₙ-aryl, in which the heterocycloalkyl in the ring contains at least one atom the same or different, of the following group of nitrogen, oxygen, or sulfur and may be made up of one or more —(CO)— or —SO₂— groups in the ring and may contain one or more double bonds in the ring,
R¹⁶ stands for hydrogen or for C₂-C₄-alkenyl, cyclopropyl or C₂-C₁₀-heterocycloalkyl substituted once or several times, the same or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹⁴ group, or for C₁-C₄-alkyl substituted once or several times, the same or different, with C₁-C₄-alkoxy, cyano, cyclopropyl, halogen, hydroxy or with the —NR¹⁰R¹¹, —O—(CO)—R⁵, —(SO₂)—R¹⁴ or —O—(SO₂)—R¹⁴ group or stands for methyl substituted once or several times, the same or different, with C₂-C₁₀-heterocycloalkyl or heteroaryl, in which the heterocycloalkyl in the ring contains at least one atom the same or different, of the following group of nitrogen, oxygen, or sulfur and may be made up of one or more —(CO)— or —SO₂— groups in the ring and may contain one or more double bonds in the ring, and the ring itself may be substituted once or several times, the same or different, with halogen, cyano, hydroxy, aryl or with the —(CO)—R⁵, —(CO)—O—R¹², —(SO₂)—R¹⁴, —NR¹²R¹³ group or once or several times, the same or different, with halogen, hydroxy, C₁-C₃-alkylthio or phenyl substituted C₁-C₃-alkyl, in which the aryl itself may once or several times, the same or different, be substituted with halogen, C₁-C₃-alkyl or C₁-C₃-alkoxy, or stands for C₁-C₄-alkyl substituted once or several times, the same or different, with C₂-C₁₀-heterocycloalkyl, or stands for C₂-C₄-alkyl substituted once or several times, the same or different, with C₁-C₄-alkoxy-C₁-C₄-alkoxy, in which the heterocycloalkyl in the ring contains at least one atom the same or different, of the following group of nitrogen, oxygen, or sulfur and may be made up of one or more —(CO)— or —SO₂— groups in the ring and may contain one or more double bonds in the ring and the ring itself may be substituted with C₁-C₃-alkyl substituted once or several times, the same or different with halogen, cyano, hydroxy, aryl or with the —(CO)—R⁵, —(CO)—O—R¹², —(SO₂)—R¹⁴, —NR¹²R¹³ group, or once or several times, the same or different, with halogen, cyano, hydroxy, aryl or with the —(CO)—R⁵, —(CO)—O—R¹², —(SO₂)—R¹⁴, —NR¹²R¹³ group, or is substituted once or several times, the same or different, with halogen, hydroxy, C₁-C₃-alkylthio or phenyl substituted C₁-C₃-alkyl, in which the aryl itself can be substituted once or several times, the same or different, with halogen, C₁-C₃-alkyl or C₁-C₃-alkoxy, and
n stands for 1-4,
as well as their solvents, hydrates, diastereomers, enantiomers, and salts.

2. Compounds of general formula I, according to claim 1,
in which the meaning is as follows:
T¹, T² and T³ stand independently of one another for —CH═ or —N═
R³ stands for K, L, or M,
X stands for halogen, hydroxy or for the —OR⁶, —NR¹⁰R¹¹ group or for C₂-C₁₀-heterocycloalkyl, in which the heterocycloalkyl in the ring contains at least one atom, equal or different, from the following group of nitrogen, oxygen, or sulfur and may also be made up of one or more —(CO)—, —(C═S)— or —SO₂— groups in the ring, and may be contained in one or more double bonds in the ring, and the ring itself may be substituted C₁-C₃-alkyl substituted once or several times, equal or different, substituted with cyano, halogen, hydroxy, aryl or with the —(CO)—R⁵, —NR¹²R¹³ group or substituted once or several times, equal or different, with halogen, hydroxy or C₁-C₃-alkylthio, in which the aryl itself may be substituted once or several times, equal or different, with cyano, halogen or C₁-C₃-alkoxy,
L stands for the group —O—R⁷, —O—(CH₂)ₙ—(CO)—NH—R⁸ or —O—(CH₂)ₙ—(CO)—O—R⁸,
R⁹ stands for C₁-C₅-alkyl, C₂-C₄-alkenyl, cyclopropyl or C₂-C₁₀-heterocycloalkyl substituted once or several times, equal or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹⁴ group, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself may be substituted once or several times, equal or different, with halogen, cyano, hydroxy, aryl or with the —(CO)—R⁵, —(CO)—O—R¹², —(SO₂)—R¹⁴, —NR¹²R¹³ group or substituted once or several times, equal or different, with halogen, hydroxy, C₁-C₃-alkylthio or phenyl substituted C₁-C₃-Alkyl, in which the aryl itself may once or several times, equal or different, substituted with halogen or C₁-C₃-alkoxy,
R¹⁶ stands for hydrogen or for C₂-C₄-alkenyl, cyclopropyl or C₂-C₁₀-heterocycloalkyl substituted once or several times, equal or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹⁴ group, or for C₁-C₄-alkyl substituted once or several times, equal or different, with C₁-C₄-alkoxy, cyano, cyclopropyl, halogen, hydroxy or with the —NR¹⁰R¹¹, —O—(CO)—R⁵, —(SO₂)—R¹⁴ or —O—(SO₂)—R¹⁴ group, or stands for methyl substituted once or several times, equal or different, with C₂-C₁₀-heterocycloalkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself may be substituted C₁-C₃-alkyl substituted once or several times, equal or different, with halogen, cyano, hydroxy, aryl or with the —(CO)—R⁵, —(CO)—O—R¹², —(SO₂)—R¹⁴, —NR¹²R¹³ group, or once or several times, equal or different, with halogen, hydroxy, C₁-C₃-alkylthio or phenyl in which
the aryl itself once or several times, equal or different, may be substituted with halogen or C₁-C₃-alkoxy,
or stands for C₁-C₄-alkyl substituted once or several times, equal or different, with C₂-C₁₀-heterocycloalkyl, or stands for C₂-C₄-alkyl substituted once or several times, equal or different, with C₁-C₄-alkoxy-C₁-C₄-alkoxy, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself may be substituted once or several times, equal or different, with halogen, cyano, hydroxy, aryl or with the —(CO)—R⁵, —(CO)—O—R¹², —(SO₂)—R¹⁴, —NR¹²R¹³ group, or once or several times, equal or different, with halogen, hydroxy, C₁-C₃-alkylthio or phenyl substituted C₁-C₃-alkyl, in which the aryl itself may once or several times, equal or different may be substituted with halogen or C₁-C₃-alkoxy, and
as well as their solvents, hydrates, diastereomers, enantiomers, and salts.

3. Compounds of general formula I, according to claim 1, in which the meaning is as follows:
R⁷ stands for C₁-C₃-alkyl substituted once or several times, equal or different, with —NR¹²R¹³ or C₂-C₁₀-heterocycloalkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring,
R⁹ stands for C₁-C₅-alkyl, C₂-C₄-alkenyl, cyclopropyl or C₂-C₁₀-heterocycloalkyl substituted if need be once or several times, equal or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹² groups, in which the heterocycloalkyl in the ring contains at least one atom equal or different, of the following group of nitrogen, oxygen, or sulfur and may be made up of one or more —(CO)— or —SO₂— groups in the ring and if may contain one or more double bonds in the ring, and the ring itself may be substituted if need be once or several times, equal or different, with halogen, cyano, hydroxy, phenyl, which itself may be substituted with if need be once or several times, equal or different, with halogen or C₁-C₃-alkoxy, or with the —(CO)—R⁵, —(CO)—O—R¹², —(SO₂)—R¹⁴NR¹²R¹³ group, or once or several times, equal or different, with halogen, hydroxy, C₁-C₃-alkylthio or phenyl substituted C₁-C₃-alkyl,
R¹⁰ and R¹¹ stand for independent of one another C₁-C₅-alkyl, C₂-C₁₀-heterocycloalkyl, aryl or heteroaryl substituted if need be once or several times, equal or different, with halogen, C₁-C₃-alkyl, C₁-C₃-alkoxy, substituted C₁-C₅-alkyl, C₂-C₁₀-heterocycloalkyl, aryl or heteroaryl, in which the heterocycloalkyl in the ring contains at least one atom equal or different, of the following group of nitrogen, oxygen, or sulfur and may be made up of one or more —(CO)— or —SO₂— groups in the ring and if may contain one or more double bonds in the ring,
R¹⁴ stands for C₁-C₃-alkyl or for phenyl, and
n stands for 1-4,
as well as their solvents, hydrates, diastereomers, enantiomers, and salts.

4. Compounds of general formula I, according to claim 1, in which the meaning is as follows:
R¹ stands for methyl, ethyl, isopropyl or cyclopropyl substituted if need be once or several times, equal or different, with halogen,
R² stands for methyl, ethyl, allyl, property or at least hydroxyethyl substituted once with methyl and substituted if need be once or several times, equal or different, with cyano, cyclopropyl, ethinyl or halogen,
X stands for halogen, hydroxy or for the —OR⁶, —NR¹⁰R¹¹ group or for azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, or tetrahydrochinolinyl, in which pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves can be substituted if need be once or several times, equal or different, with halogen, hydroxy, phenyl, which itself can be substituted if need be once or several times, equal or different, with halogen or C₁-C₃-alkoxy, or can be substituted if need be once or several times, equal or different, with the —(CO)—R⁵, —NR¹²R¹³ group or substituted if need be once or several times, equal or different with cyano, halogen, hydroxy or C₁-C₃-alkylthio substituted C₁-C₃-Alkyl,
R⁴ stands for hydrogen, cyano or halogen or for methyl substituted if need be once or several times, equal or different, with halogen,
R⁵ stands for methyl, ethyl, tert.-butyl, phenyl or —NH₂,
R⁶ stands for —SO₂-Methyl,
R⁷ stands for C₁-C₃-alkyl substituted if need be once or several times, equal or different, with —N(C₁-C₃-alkyl)₂, pyrrolidinyl, morpholinyl, or piperidinyl
R⁸ stands for methyl, ethyl, allyl or propargyl substituted if need be once or several times, equal or different, with cyano, cyclopropyl or halogen,
R⁹ stands for methyl, ethyl, isopropyl, isobutyl, tert.-butyl, ethenyl, cyclopropyl, tetrahydropyranyl, or tetrahydrofuranyl, isobutyl, tert.-butyl, ethenyl, cyclopropyl, tetrahydropyranyl or tetrahydrofuranyl, substituted if need be once or several times, equal or different, with C₁-C₄-alkoxy, C₁-C₄-alkoxy-C₁-C₄-alkoxy, pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl, cyano, cyclopropyl, halogen, hydroxy or with the —NR¹⁰R¹¹, —O—(CO)—R⁵, —(SO₂)—R¹⁴ or —O—(SO₂)—C₁-C₃-alkyl group, in which pyrrolidinyl, piperidinyl, piperazinyl, thiomorpholinyl, benzopyrrolidinyl, tetrahydrochinolinyl, tetrahydrooxazolyl, piperazinonyl, tetrahydrothiazolyl, tetrahydroimidazolonyl, benzomorpholinyl, tetrahydrotriazolthionyl, morpholinyl, tetrahydroisochinolinyl, octahydroisochinolinyl themselves may be substituted if need be once or several times, equal or different, with halogen, hydroxy, phenyl or C₁-C₃-Alkoxy, or with the —(CO)—R⁵, —(CO)—O—R⁵, —(SO₂)—R¹⁴, —N(CH₃)₂ group, or substituted if need be once or several times, equal or different with halogen, hydroxy, methylthio, or phenyl substituted methyl or ethyl,
R¹⁰ und R¹¹ stand independent of one another for C₁-C₅-alkyl, pyrrolidinyl, phenyl or pyridinyl substituted if need be once or several times, equal or different, with halogen, C₁-C₃-alkyl or C₁-C₃-alkoxy,
R¹² and R¹³ stand independent of one another for hydrogen or for methyl, ethyl, or isopropyl,
R¹⁴ stands for C₁-C₄-alkyl or for phenyl, and
n stands for 1 or 2,
as well as their solvents, hydrates, diastereomers, enantiomers, and salts.

5. Compounds of general formula I, according to claim 1, in which the meaning is as follows:
U stands for —CH═, —CF═, —C(CH₃)═ or —N═,
R¹ stands for methyl, ethyl, isopropyl or cyclopropyl substituted if need be once or several times, equal or different, with fluorine,
R² stands for methyl, ethyl, allyl, propargyl or at least hydroxyethyl substituted at least once with methyl, substituted if need be once or several times, equal or different, with cyano, cyclopropyl, ethinyl or fluorine,
K stands for methyl, ethyl or ethenyl substituted if need be once or several times, equal or different with X,
X stands for halogen, hydroxy or for the —O—SO₂-methyl group or for pyrrolidinyl, morpholinyl, thiomorpholinyl, piperidinyl or octahydroisochinolinyl, in which methyl can be substituted if need be once or several times, equal or different, with pyrrolidinyl, morpholinyl, thiomorpholinyl, piperidinyl or octahydroisochinolinyl, itself substituted if need be once or several times, equal or different with halogen, hydroxy, phenyl or with methyl substituted if need be once or several times, equal or different with halogen,
L stands for the group —O—R⁷, —O—(CH₂)ₙ—(CO)—NH—R⁸ or —O—(CH₂)ₙ—(CO)—O—R⁸,
M stands for the group —NH—R⁹, —NH—(CO)—OH, —NH—(CO)—O—R⁹ or —NR¹²—(CO)—R¹⁶,
R⁷ stands for ethyl substituted if need be once or several times, equal or different with —N(C₁-C₃-alkyl)₂, pyrrolidinyl, morpholinyl or piperidinyl,
R⁸ stands for methyl, ethyl, allyl or propargyl substituted if need be once or several times, equal or different with cyano, cyclopropyl or fluorine
R⁹ stands for methyl, ethyl, isopropyl, isobutyl, tert.-butyl, ethenyl, cyclopropyl, tetrahydropyranyl or tetrahydrofuranyl substituted if need be once or several times, equal or different, with C₁-C₄-alkoxy, C₁-C₄-alkoxy-C₁-C₄-alkoxy, pyrrolidinyl, piperidinyl, piperazinyl, morpholinyl, thiomorpholinyl, cyano, cyclopropyl, halogen, hydroxy or with the —N(C₁-C₃-alkyl)₂, —O—(CO)—(C₁-C₃-alkyl) or —O—(SO₂)—C₁-C₃— alkyl-group, in which pyrrolidinyl, piperidinyl, piperazinyl, morpholinyl, thiomorpholinyl can themselves be substituted if need be once or several times, equal or different, with halogen or with the —(CO)—C₁-C₄-alkyl, —(CO)—O—C₁-C₄-alkyl, —(SO₂)—C₁-C₃-alkyl, —(SO₂)-phenyl, —N(C₁-C₃-alkyl)₂ group, or substituted if need be once or several times, equal or different, with halogen, hydroxy or C₁-C₃-alkylthio substituted methyl or ethyl,
as well as their solvents, hydrates, diastereomers, enantiomers, and salts.

6. Compounds of general formula I, according to claim 1, in which the meaning is as follows:
R¹ stands for ethyl,
X stands for iodine, hydroxy or for the —O—SO₂-methyl group or for pyrrolidinyl, morpholinyl, thiomorpholinyl, piperidinyl or octahydroisochinolinyl, in which pyrrolidinyl, morpholinyl, thiomorpholinyl, piperidinyl or octahydroisochinolinyl can themselves be substituted if need be once or several times, equal or different, with halogen, hydroxy, phenyl or methyl substituted if need be once or several times, equal or different, with halogen,
R⁷ stands for ethyl substituted if need be once or several times, equal or different with —N(CH₃)₂, pyrrolidinyl, morpholinyl or piperidinyl,
R⁹ stands for methyl, ethyl, isopropyl, isobutyl, tert.-butyl, ethenyl, cyclopropyl, tetrahydropyranyl or tetrahydrofuranyl, substituted if need be once or several times, equal or different, with methoxy, ethoxy, butoxy-ethoxy, methoxy-ethoxy, pyrrolidinyl, piperidinyl, piperazinyl, morpholinyl, thiomorpholinyl, cyano, cyclopropyl, chlorine, fluorine, hydroxy or the —N(CH₃)₂, —N(CH₃)(C₂H₅), —O—(CO)—(CH₃) or —O—(SO₂)-methyl group, in which pyrrolidinyl, piperidinyl, piperazinyl, morpholinyl, thiomorpholinyl can themselves be substituted if need be once or several times, equal or different, with fluorine, or with the —(CO)—CH₃, —(CO)—C₂H₅, —(CO)—C(CH₃)₃, —(CO)—O—C(CH₃)₃, —(SO₂)—CH₃, —(SO₂)-phenyl, —N(CH₃)₂ group, or can be substituted if need be once or several times, equal or different, with fluorine, hydroxy, or methylthio substituted methyl or ethyl,
as well as their solvents, hydrates, diastereomers, enantiomers, and salts.

7. Compounds of general formula I, according to claim 1, in which the meaning is as follows:
R¹⁶ stands for C₂-C₁₀-heterocycloalkyl substituted methyl substituted if need be once or several times, equal or different, with C₁-C₄-alkoxy, cyano, cyclopropyl, halogen, hydroxy or with the —NR¹⁰R¹¹, —O—(CO)—R⁵, —(SO₂)—R¹⁴ or —O—(SO₂)—R¹⁴ substituted C₁-C₄-alkyl group, in which the heterocycloalkyl in the ring contains at least one atom equal or different, of the following group of nitrogen, oxygen, or sulfur and may
be made up of one or more —(CO)— or —SO₂— groups in the ring and may contain one or more double bonds in the ring, and the ring itself may be substituted if need be once or several times, equal or different, with halogen, cyano, hydroxy, aryl or with the —(CO)—R⁵, —(CO)—O—R¹²—(SO₂)—R¹⁴, NR¹²R¹³ group or may be substituted with C₁-C₃-alkoxy which itself may be substituted if need be once or several times, equal or different, with halogen, hydroxy, C₁-C₃-alkylthio or phenyl substituted C₁-C₃-alkyl, in which the aryl itself can be substituted if need be once or several times, equal or different, with halogen or C₁-C₃-alkoxy,
as well as their solvents, hydrates, diastereomers, enantiomers, and salts.

8. Compounds of general formula I, according to claim 7,
R¹⁶ stands for C₁-C₄-alkyl substituted if need be once or several times, equal or different, with the —NR¹⁰R¹¹ group, or methyl substituted if need be once or several times, equal or different, with C₂-C₁₀-heterocycloalkyl, in which in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself may be substituted if need be once or several times, equal or different, with halogen, cyano, hydroxy, aryl or with the —(CO)—R⁵, —(CO)—O—R¹², —(SO₂)—R¹⁴, —NR¹²R¹³ group, or substituted if need be once or several times, equal or different with halogen, hydroxy, C₁-C₃-alkylthio or phenyl substituted C₁-C₃-alkyl, in which the aryl itself can be substituted if need be once or several times, equal or different with halogen or C₁-C₃-alkoxy,
as well as their solvents, hydrates, diastereomers, enantiomers, and salts.

9. Compounds of general formula I, according to claim 1, in which the meaning is as follows:
K stands for C₁-C₃-alkyl substituted if need be once or several times, equal or different, with P or C₂-C₄-alkenyl substituted if need be once or several times, equal or different, with X,
P stands for the —OR⁶, —NR¹⁸R¹⁹. C₂-C₅-heterocycloalkyl group or for C₆-C₁₀ heterocycloalkyl, in which the C₂-C₅-heterocycloalkyl and the C₆-C₁₀ heterocycloalkyl in the ring contain at least one atom equal or different, of the following group of nitrogen, oxygen, or sulfur and may be made up of one or more —(CO)— or —SO₂— groups in the ring and may contain one or more double bonds in the ring, and the ring of C₂-C₅-Heterocycloalkyl itself is substituted once or several times, equal or different, with cyano, halogen, hydroxy, aryl or with the —(CO)—R⁵ group or substituted once or several times, equal or different, with halogen or C₁-C₃-Alkylthio substituted C₁-C₃-alkyl, in which the aryl itself can be substituted if need be once or several times, equal or different, with cyano, halogen or C₁-C₃-alkoxy, and the ring of the C₆-C₁₀-heterocycloalkyl itself can be substituted if need be once or several times, equal or different, with cyano, halogen, hydroxy, aryl or with the —(CO)—R⁵, —NR¹²R¹³ group or substituted if need be once or several times, equal or different, with halogen, hydroxy or C₁-C₃-alkylthio substituted C₁-C₃-alkyl, in which the aryl itself can be substituted if need be once or several times, equal or different, with cyano, halogen or C₁-C₃-alkoxy,
L stands for the group —O—R⁷, —O—(CH₂)ₙ—(CO)—NH—R¹⁷, —O—(CH₂)ₙ—(CO)—R¹⁵ or —O—(CH₂)ₙ—(CO)—O—R⁸,
R⁷ stands for C₁-C₃-Alkyl substituted if need be once or several times, equal or different, with C₆-C₁₀-Heterocycloalkyl, in which the C₆-C₁₀. Heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself can be substituted if need be once or several times, equal or different, with halogen, aryl or once or several times, equal or different, with halogen substituted C₁-C₃-Alkyl, or
stands for C₁-C₃-alkyl substituted once or several times, equal or different, with C₂-C₅-heterocycloalkyl, in which the C₂-C₅-heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself is substituted if need be once or several times, equal or different, with halogen, aryl, or substituted once or several times, equal or different, with halogen substituted C₁-C₃-alkyl,
R¹⁶ stands for hydrogen, C₂-C₄-alkenyl, cyclopropyl, C₂-C₅-heterocycloalkyl or C₆-C₁₀-heterocycloalkyl or methyl substituted with heteroaryl or for C₁-C₄-alkyl, C₂-C₄-alkenyl, cyclopropyl, C₂-C₅-heterocycloalkyl or C₆-C₁₀-heterocycloalkyl substituted once or several times, equal or different, with C₁-C₄-alkoxy, C₂-C₅-heterocycloalkyl, C₆-C₁₀-heterocycloalkyl, cyano, cyclopropyl or the —NR¹⁸R¹⁹, —O—(CO)—R⁵, —(SO₂)—R¹⁴ or —O—(SO₂)—R¹⁴ group, in which the C₂-C₅-heterocycloalkyl and the C₆-C₁₀-heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring of C₂-C₅-heterocycloalkyl itself is substituted once or several times, equal or different, with halogen, cyano, hydroxy, aryl or with the —(CO)—R⁵, —(CO)—O—R¹², —(SO₂)—R¹⁴ group, or substituted once or several times, equal or different, with halogen, C₁-C₃-alkylthio or phenyl substituted C₁-C₃-alkyl, in which the aryl itself can be substituted once or several times, equal or different, with halogen, C₁-C₃-alkyl or C₁-C₃-alkoxy,
R¹⁷ stands for C₁-C₃-alkyl substituted once or several times, equal or different, with halogen or cyano, or if need be substituted once or several times, equal or different, with halogen, cyclopropyl or cyano substituted C₃-C₄-alkenyl or C₃-C₄-alkinyl,
R¹⁸ und R¹⁹ stands for independent of one another C₁-C₅-alkyl, C₂-C₁₀-heterocycloalkyl, aryl, —(CH₂)ₙ-aryl or heteroaryl if need be substituted once or several times, equal or different, with halogen, C₁-C₃-alkyl, C₁-C₃-alkoxy, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, in which
either R¹⁸ or R¹⁹ stands for C₂-C₁₀-heterocycloalkyl, —(CH₂)ₙ-aryl, or a heteroaryl, or for a C₂-C₁₀-heterocycloalkyl, —(CH₂)ₙ-aryl or heteroaryl if need be substituted once or several times, equal or different, with halogen, C₁-C₃-alkyl, C₁-C₃-alkoxy, or stands for a C₁-C₅-alkyl substituted once or several times, equal or different, with C₁-C₃-alkoxy, or for an aryl substituted once or several times, equal or different, with C₁-C₃-alkyl, C₁-C₃-alkoxy, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring,
as well as their solvents, hydrates, diastereomers, enantiomers, and salts.

10. Compounds of general formula I, according to claim 9, in which the meaning is as follows:
in which
T¹, T² and T³ stand independently of one another for —CH═ or —N═
R³ stands for K, L, or M,
P stands for the —OR⁶, —NR¹⁸R¹⁹. C₂-C₅-heterocycloalkyl group or for C₆-C₁₀ heterocycloalkyl, in which the C₂-C₅-heterocycloalkyl and the C₆-C₁₀ heterocycloalkyl in the ring contain at least one atom equal or different, of the following group of nitrogen, oxygen, or sulfur and may be made up of one or more —(CO)— or —SO₂— groups in the ring and may contain one or more double bonds in the ring, and the ring of the C₂-C₅-heterocycloalkyl itself is substituted once or several times, equal or different, with cyano, halogen, hydroxy, aryl or with the —(CO)—R⁵ group or substituted once or several times, equal or different, with halogen or C₁-C₃-Alkylthio substituted C₁-C₃-alkyl, in which the aryl itself may be substituted if need be once or several times, equal or different, with cyano, halogen or C₁-C₃-alkoxy,
L stands for the —O—R⁷, —O—(CH₂)ₙ—(CO)—NH—R¹⁷ or —O—(CH₂)ₙ—(CO)—O—R⁸ group,
R⁹ stands for C₁-C₄-alkyl, C₂-C₄-alkenyl, cyclopropyl or C₂-C₁₀-heterocycloalkyl, substituted if need be once or several times, equal or different, with C₁-C₄-alkoxy, C₁-C₄-alkoxy-C₁-C₄-alkoxy, C₂-C₁₀-heterocycloalkyl, cyano, cyclopropyl, halogen, hydroxy or with the —NR¹⁰R¹¹, —O—(CO)—R⁵, —(SO₂)—R¹⁴ or —O—(SO₂)—R¹⁴ group, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself if need be once or several times, equal or different can be substituted with halogen, cyano, hydroxy, aryl or with the —(CO)—R⁵, —(CO)—O—R¹², —(SO₂)—R¹⁴, —NR¹²R¹³ group or if need be once or several times, equal or different, can be substituted with halogen, hydroxy, C₁-C₃-alkylthio or phenyl substituted C₁-C₃-alkyl, in which the aryl itself can be substituted if need be once or several times, equal or different, with halogen or C₁-C₃-alkoxy,
R¹⁶ stands for hydrogen, C₂-C₄-alkenyl, cyclopropyl, C₂-C₅-heterocycloalkyl or C₆-C₁₀-heterocycloalkyl or for C₁-C₄-alkyl, C₂-C₄-alkenyl, cyclopropyl, C₂-C₅-heterocycloalkyl or C₆-C₁₀-Heterocycloalkyl substituted once or several times, equal or different, with C₁-C₄-alkoxy, C₂-C₈-heterocycloalkyl, C₆-C₁₀-heterocycloalkyl, cyano, cyclopropyl or with the —NR¹⁸R¹⁹, —O—(CO)—R⁵, —(SO₂)—R¹⁴ or —O—(SO₂)—R¹⁴ substituted C₁-C₄-Alkyl group, in which the C₂-C₅-heterocycloalkyl and the C₆-C₁₀-heterocycloalkyl in the ring contain at least one atom equal or different, of the following group of nitrogen, oxygen, or sulfur and may be made up of one or more —(CO)— or —SO₂— groups in the ring and may contain one or more double bonds in the ring, and the ring of the C₂-C₅-heterocycloalkyl itself is substituted once or several times, equal or different, with halogen, cyano, hydroxy, aryl or with the —CO)—R⁵, —(CO)—O—R¹², —(SO₂)—R¹⁴, —NR¹²R¹³ group, or is substituted once or several times, equal or different, with halogen, hydroxy, C₁-C₃-alkylthio or phenyl substituted C₁-C₃-alkyl, in which the aryl itself if need be once or several times, equal or different, can be substituted with halogen or C₁-C₃-alkoxy,
as well as their solvents, hydrates, diastereomers, enantiomers, and salts.

11. Compounds of general formula I, according to claim 1, in which
R³ stands for K, or L,
K stands for C₁-C₃-alkyl substituted be once or several times, equal or different, with X, in which the C₁-C₃-Alkyl can be substituted if need by once or several times, equal or different, with hydroxy or halogen,
X stands for NR¹⁰R¹¹ or for C₂-C₁₀-heterocycloalkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself if need be once or several times, equal or different, can be substituted with cyano, halogen, hydroxy, aryl or with the —(CO)—R⁵, —NR¹²R¹³ group or substituted with if need be once or several times, equal or different, halogen, hydroxy or C₁-C₃-alkylthio substituted C₁-C₃-alkyl, in which the aryl itself can be substituted if need be once or several times, equal or different, with cyano, halogen or C₁-C₃-alkoxy,
L stands for the —O—R⁷ group,
R⁷ stands for C₁-C₃-alkyl substituted once or several times, equal or different, with —NR¹²R¹³ or C₂-C₁₀-heterocycloalkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself if need be once or several times, equal or different, can be substituted with halogen, aryl or substituted if need be once or several times, equal or different, with halogen substituted C₁-C₃-Alkyl,
as well as their solvents, hydrates, diastereomers, enantiomers, and salts.

12. Compounds of general formula I, according to claim 11, in which the meaning is as follows:
X stands for —N(C₁-C₃-Alkyl)₂ or for azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl or tetrahydrochinolinyl, in which azetidinyl, pyrrolidinyl, morpholinyl, thiomorpholinyl, piperidinyl, octahydroisochinolinyl, benzopyrrolidinyl, piperazinyl, tetrahydrooxazolyl, piperazinonyl, tetrahydrothiazolyl, tetrahydroimidazolonyl, benzomorpholinyl, triazinthionyl, tetrahydroisochinolinyl, tetrahydrochinolinyl themselves may be substituted if need be once or several times, equal or different, with halogen, hydroxy, phenyl, that itself may be substituted if need be once or several times, equal or different, with halogen or C₁-C₃-alkoxy, or with the —(CO)—R⁵ group or substituted once or several times, equal or different, with cyano, halogen or C₁-C₃-alkylthio substituted C₁-C₃-alkyl.
R⁷ stands for C₁-C₃-alkyl substituted once or several times, equal or different, with —N(C₁-C₃-Alkyl)₂ or C₂-C₁₀-heterocycloalkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring,
as well as their solvents, hydrates, diastereomers, enantiomers, and salts.

13. Compounds of general formula I, according to claim 1, in which the meaning is as follows:
R³ stands for M,
M stands for the —NR¹²—(CO)—R¹⁶ group,
R¹⁶ stands for methyl substituted if need be once or several times, equal or different, with C₁-C₄-alkoxy, C₂-C₁₀-heterocycloalkyl, heteroaryl, cyano, cyclopropyl, halogen, hydroxy or with the —NR¹⁰R¹¹, —O—(CO)—R⁵, —(SO₂)—R¹⁴ or —O—(SO₂)—R¹⁴ group, in which the methyl itself can be substituted if need be once or several times, equal or different, with C₁ to C₃-alkyl, in which the heterocycloalkyl in the ring contains at least one atom equal or different, of the following group of nitrogen, oxygen, or sulfur and may be made up of one or more —(CO)—, —(C═S)— or —SO₂— groups in the ring and may contain one or more double bonds in the ring, and the ring itself if need be once or several times, equal or different, can be substituted with halogen, cyano, hydroxy, aryl or with the —(CO)—R⁵, —(CO)—O—R¹², —(SO₂)—R¹⁴, —NR¹²R¹³ group or can be substituted if need be once or several times, equal or different, with halogen, hydroxy, C₁-C₃-alkylthio or phenyl substituted C₁-C₃-alkyl, in which the aryl itself if need be once or several times, equal or different, can be substituted with halogen, C₁-C₃-alkyl or C₁-C₃-alkoxy,
as well as their solvents, hydrates, diastereomers, enantiomers, and salts.

14. Compounds of general formula I, according to claim 13, in which the meaning is as follows:
R¹⁶ stands for methyl substituted once or several times, equal or different, with C₂-C₁₀-heterocycloalkyl, heteroaryl or with the —NR¹⁰R¹¹ group, in which the methyl itself can be substituted if need be once or several times, equal or different, with C₁ to C₃-alkyl, in which the heterocycloalkyl in the ring contains at least one atom equal or different, of the following group of nitrogen, oxygen, or sulfur and may be made up of one or more —(CO)— or —SO₂— groups in the ring and may contain one or more double bonds in the ring, and the ring itself if need be once or several times, equal or different, can be substituted with halogen, cyano, hydroxy, aryl or with the —(CO)—R⁵, —(CO)—O—R¹², —(SO₂)—R¹⁴, —NR¹²R¹³ group or can be substituted if need be once or several times, equal or different, with halogen, hydroxy, C₁-C₃-alkylthio or phenyl substituted C₁-C₃-alkyl, in which the aryl itself if need be once or several times, equal or different, can be substituted with halogen, C₁-C₃-alkyl or C₁-C₃-alkoxy,
as well as their solvents, hydrates, diastereomers, enantiomers, and salts.

15. Compounds of the general formulas II or IV 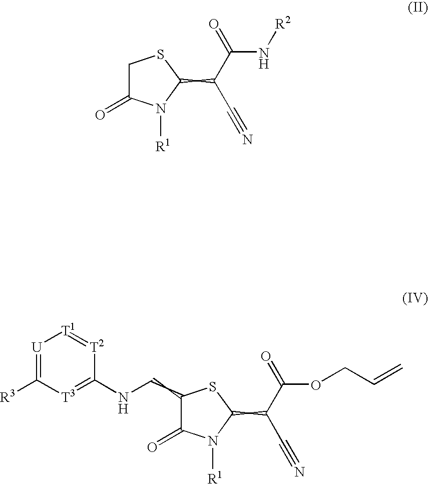
in which
R¹, R², R³, U, T¹, T² and T³ which in the general Formula I, according to one of the claims from 1 to 14, have the cited meaning, as do their solvents, hydrates, diastereomers, enantiomers, and salts as intermediate products for the creation of the compounds of general formula (I).

16. Compounds of general formula II according to claim 15 with the following formulas:
2-cyano-N-ethyl-2-[3-ethyl-4-oxo-thiazolidin-(2-(E or Z))-ylidene]-acetamide,
2-cyano-2-[3-ethyl-4-oxo-thiazolidin-(2-(E or Z))-ylidene]-N-(2,2,2-trifluoro-ethyl)-acetamide,
2-cyano-2-[3-ethyl-4-oxo-thiazolidin-(2-(E or Z))-ylidene]-N-prop-2-ynyl-acetamide or
2-cyano-N-cyanomethyl-2-[3-ethyl-4-oxo-thiazolidin-(2-(E or Z))-ylidene]-acetamide
2-cyano-N-(2,2-difluoro-ethyl)-2-[3-ethyl-4-oxo-thiazolidin-(2-(E or Z))-ylidene]-acetamide
2-cyano-2-[3-ethyl-4-oxo-thiazolidin-(2-(E or Z))-ylidene]-N-(2-hydroxy-1,1-dimethyl-ethyl)-acetamide
2-cyano-2-[3-ethyl-4-oxo-thiazolidin-(2-(E or Z))-ylidene]-N-(2-fluoro-ethyl)-acetamide as well as their solvents, hydrates, diastereomers, enantiomers, and salts as intermediate products for the creation of the compounds of general formula (I).

17. Compounds of the general formulas (II) or (IV) according to claim 15 or compounds according to claim 16 for use as intermediate products for the creation of compounds of general formula (I).

18. Compounds of the general formulas (II) or (IV) according to claim 15 or compounds according to claim 16 for use as intermediate products for the creation of compounds of general formula (I).

19. Medications that contain at least one compound in accordance with claim 1.

20. The use of the compounds of general formula I, according to claim 1, for the creation of a medication.

21. Compounds according to claim 1 or medications with appropriate materials for formulation and delivery.

22. The process for creation of compounds of general formula I, in which compounds of general formula II and compounds of general formula III, 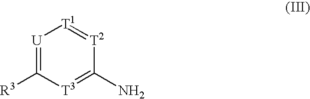
in which
R³, U, T¹, T² und T³ have the same meaning as R³³, U, T¹, T² und T³ according to claim 1, are heated in a formic acid orthoester with three equal or different possibly connected alkoxy- or aryloxyresters or substituted with halogen and if necessary with a polar solvent,
or
Compounds of general formula IV 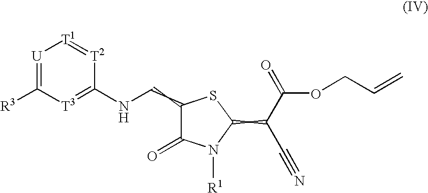
in which
R³, U, T¹, T² und T³ have the same meaning as R³, U, T¹, T² und T³ according to claim 1, are moved together with an allyl receptor and a catalytic converter into a non-protein solvent, and after a completed initial partial reaction are transformed with a coupling agent, a base, and R²—NH₂, in which R² has the same meaning as R² in claim 1, in a non-protein solvent into the compounds of general formula I.

23. A process under claim 22, in which for the creation of compounds of general formula II, compounds of general formula V, 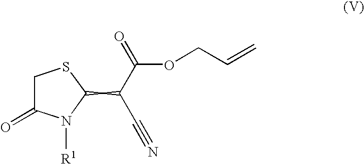
in which
R¹ has the same meaning as R¹ in claim 1, are moved together with an allyl receptor and a catalytic converter into a non-protein solvent, and after a completed initial partial reaction are transformed with a coupling agent, a base, and R²—NH₂, in which R² has the same meaning as R² in claim 1, in a non-protein solvent into the compounds of general formula I.
